(19) United States
(12) Patent Application Publication
(10) Pub. No.: US 2026/0216170 A1
(43) Pub. Date: Jul. 30, 2026

(54) TETRAHYDROISOQUINOLINE HETEROBIFUNCTIONAL BCL-XL DEGRADERS

(71) Applicant: Treeline Biosciences, Inc., Watertown, MA (US)

(72) Inventors: Evan J. Horn, San Diego, CA (US); Joshua D. Hansen, San Diego, CA (US); Matthew D. Alexander, San Diego, CA (US); Fei Huang, La Mesa, CA (US); Mark A. Nagy, Encinitas, CA (US); Adam Jamil Zahara, San Diego, CA (US)

(21) Appl. No.: 19/146,573

(22) PCT Filed: Nov. 6, 2024
(86) PCT No.: PCT/US2024/054673

§ 371 Date: Jul. 9, 2025

Related U.S. Application Data
(60) Provisional application No. 63/547,656, filed on Nov. 7, 2023.
(60) Provisional application No. 63/641,308, filed on May 1, 2024.
(60) Provisional application No. 63/704,392, filed on Oct. 7, 2024.

(51) Int. Cl. A61K 31/4709; A61K 31/438; A61P 35/00; C07D 471/04; C07D 519/00

(52) U.S. Cl. CPC A61K 31/4709; A61K 31/438; A61P 35/00; C07D 471/04; C07D 519/00

(57) ABSTRACT

This disclosure provides compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or a pharmaceutically acceptable salt thereof, that induce degradation of a BCL-X_{L} protein. These compounds are useful, for example, for treating a cancer in a subject (e.g., a human). This disclosure also provides compositions comprising the compounds, or pharmaceutically acceptable salts thereof, provided herein as well as methods of using and making the same.

### CROSS-REFERENCE TO RELATED APPLICATIONS

This application claims priority to U.S. Provisional Application Ser. No. 63/547,656, filed Nov. 7, 2023; 63/641,308, filed May 1, 2024; and 63/704,392, filed Oct. 7, 2024; each of which is incorporated by reference in its entirety herein.

### TECHNICAL FIELD

This disclosure provides compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, that induce degradation of a BCL-X_{L} protein. These compounds are useful, for example, for treating cancer in a subject (e.g., a human). This disclosure also provides compositions comprising the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, as well as methods of using and making the same.

### BACKGROUND

The BCL-2 family of proteins is involved in the regulation of cell apoptosis and includes proteins that are pro-apoptosis, pro-survival, and BH3-only. At a high level, the balance of binding of BH3-only proteins to the pro-apoptosis and pro-survival members of the BCL-2 family can determine whether a cell will undergo apoptosis. The protein BCL-X_{L}, encoded by the BCL2L1 gene, is a pro-survival member of the BCL-2 family. In many cancers, it can be desirable to initiate apoptosis of tumor cells, which may be achieved by decreasing the amount of pro-survival protein (e.g., BCL-X_{L}) available to compete for BH3-only protein binding.

### SUMMARY

This disclosure provides compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or 5 Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, that induce degradation of a BCL-X_{L} protein. These compounds are useful, for example, for treating a cancer in a subject (e.g., a human). This disclosure also provides compositions comprising the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), as well as methods of using and making the same.

Provided herein are compounds of Formula (I):

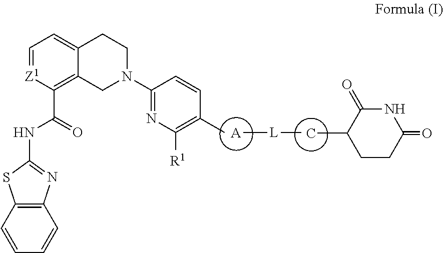

or pharmaceutically acceptable salts thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{a};
Ring A is selected from the group consisting of: (A1) and (A2):

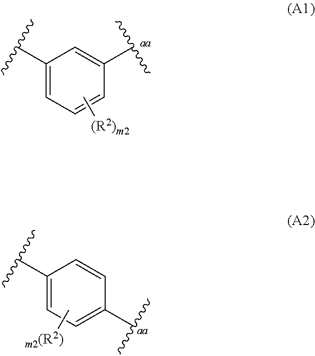

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to L;
L is -(L^{A})ₙ₁-, wherein:
n1 is an integer from 4 to 10,
one L^{A} is L^{A4};
0-3 L^{A} are independently selected L^{A3}, and
each remaining L^{A} is an independently selected L^{A1};
each L^{A1} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
each L^{A3} is independently selected from the group consisting of: —N(R^{d})—, —N(R^{b})—, —O—, —S(O)₀₋₂—, and C(═O);
L^{A4} is independently selected from the group consisting of:
(a) C₃₋₁₂ cycloalkylene or 4-15 membered heterocyclylene, each of which is optionally substituted with 1-3 R^{L4}; and
(b) phenylene or 5-6 membered heteroarylene, each of which is optionally substituted with 1-3 R^{L4},
wherein each R^{L4} is independently selected from the group consisting of R^{a} and R^{b};
Ring C is selected from the group consisting of:

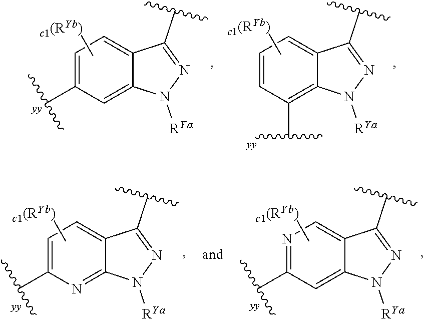

wherein:
R^{Ya} is selected from the group consisting of: H, R^{b}, and C₁₋₆ alkyl optionally substituted with 1-3 R^{c},
each R^{Yb} is independently selected from the group consisting of R^{a} and R^{b};
c1 is 0, 1, or 2; and
yy represents the point of attachment to L;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂ (C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂ (C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Also provided herein are compounds of Formula (II):

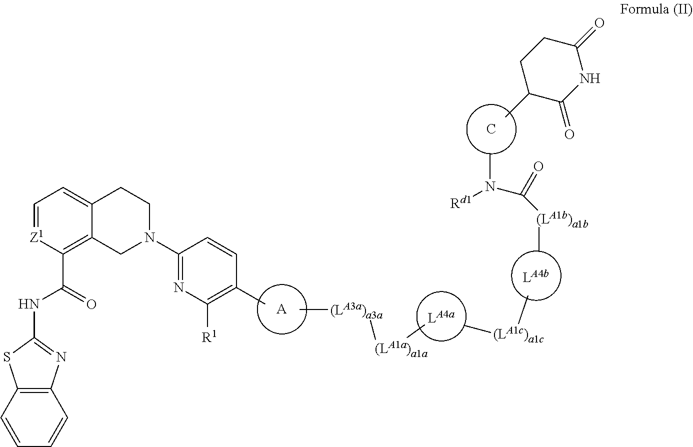

or pharmaceutically acceptable salts thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{g};
Ring A is selected from the group consisting of: (A1) and (A2):

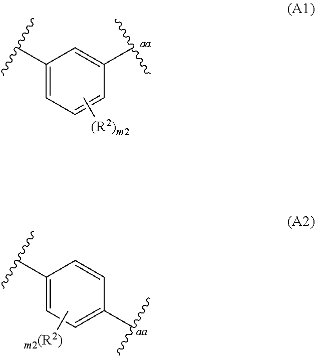

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to -(L^{A3a})ₐ₃ₐ-;
L^{A3a} is selected from the group consisting of: —N(R^{d})—, —O—, —S(O)₀₋₂—, and C(═O);
a3a is 0 or 1;
each L^{A1a} L^{A1b}, and L^{A1c} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
a1a, a1b, and a1c are each independently 0, 1, or 2;
L^{A4a} and L^{A4b} are independently selected from the group consisting of: C₃₋₈ cycloalkylene and 4-12 membered heterocyclylene, each of which is optionally substituted with 1-3 R^{L4};
wherein each R^{L4} is independently selected from the group consisting of: —F, CN, OH, C₁₋₃ alkoxy, and C₁₋₃ alkyl optionally substituted with 1-3 R^{c};
R^{d1} is —H or C₁₋₃ alkyl;
Ring C is selected from the group consisting of:

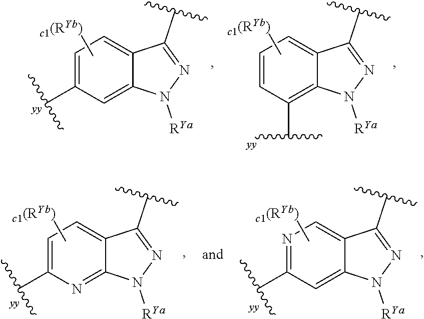

wherein:
R^{Ya} is selected from the group consisting of: H and C₁₋₆ alkyl optionally substituted with 1-3 R^{c},
each R^{Yb} is independently selected from the group consisting of: F, C₁₋₃ alkoxy, or C₁₋₃ alkyl optionally substituted with 1-3 R^{c};
c1 is 0, 1, or 2; and
yy represents the point of attachment to —N(R^{d1})—;
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂ (C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Also provided herein are pharmaceutical compositions comprising a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or a pharmaceutically acceptable salt thereof, and a pharmaceutically acceptable carrier.

Provided herein are methods for treating cancer in a subject in need thereof, the methods comprising administering to the subject a therapeutically effective amount of a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (I-a1) or (II-a2)), or Formula (A), or a pharmaceutically acceptable salt thereof, or a pharmaceutical composition as provided herein.

Also provided herein is a BCL-X_{L} protein non-covalently bound with a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (I-a1) or (II-a2)), or Formula (A), or a pharmaceutically acceptable salt thereof.

Also provided herein is a ternary complex comprising a BCL-X_{L} protein, a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In the case of conflict between the present disclosure and any content incorporated by reference, the present disclosure controls.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, that induce degradation of a BCL-X_{L} (also referred to as Bcl-xL herein) protein. These compounds are useful, for example, for treating a cancer. This disclosure also provides compositions comprising the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-hi), (I-i1), or (I-i3)) or Formula (I-2) (e.g., (I-a2), (I-b2), (I-b4), (I-c2), (I-d2), (I-e2), (I-e4), (I-2), (I-g2), (I-g4), (I-h2), or (I-i2))), Formula (II) (e.g., Formula (II-a1) or (II-a2)), or Formula (A), as well as methods of using and making the same.

Without being bound by any particular theory, it is believed that in healthy cells, pro-apoptotic effectors such as BAX and BAK can move between the cytosol and the mitochondrial outer membrane (MOM), on which Voltage Dependent Anion Channel 2 (VDAC2) can act as a receptor. Pro-survival BCL-2 family members (e.g., BCL-2, BCL-X_{L}, and MCL-1) can retrotranslocate BAX back to the cytosol. A BH3-only protein (e.g., BIM) can engage a rear site on a pro-apoptotic effector (e.g., BAX or BAK) and release the C-terminal transmembrane domain (α9) of the effector, enabling binding to the MOM. Binding of BIM to the canonical BH3 binding groove of BAX or BAK releases the N-terminus and α1 of BAX or BAK, and subsequent unfolding of the “latch” domain releases BIM from the BH3 binding groove. If a pro-survival BCL-2 family member then binds to BAX or BAK, apoptotic signaling is generally halted. However, if BAX or BAK are allowed to dimerize, then oligomerize, the MOM can be permeabilized, leading to apoptosis.

An abundance of pro-survival BCL-2 family members is sometimes thought to “prime” cells for death (e.g., via cytotoxic therapies, including BH3 mimetics). It is believed that oncogenic mutations and stresses can cause an upregulation in BH3-only proteins, thus placing a selective pressure on cancer cells for upregulation of the pro-survival BCL-2 family proteins. Thus, with more BH3-only proteins around, the cells are believed to be more sensitive to further manipulations of the BCL-2 family balance. See, e.g., Adams and Cory, Cell Death & Differentiation 25.1 (2018): 27-36, doi: 10.1038/cdd.2017.161.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, for example, small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2017/184995; WO 2019/144117; WO 2020/163823; WO 2021/078301; WO 2021/146536; WO 2021/007307; WO 2021/222114; WO 2022/169780; WO 2023/044046; WO 2023/185986; WO 2024/027706; WO 2024/179577; WO 2024/169976; WO 2024/078581; WO 2024/077023; WO 2024/067818; WO 2024/051741; WO 2024/012449; WO 2024/012557; Chamberlain and Hamann, Nature Chemical Biology 15.10 (2019): 937-944, doi: 10.1038/s41589-019-0362-y; Li and Song, Journal of Hematology & Oncology 13 (2020): 1-14, doi: 10.1186/s13045-020-00885-3; Wu, et al. Nature Structural & Molecular Biology 27.7 (2020): 605-614, doi: 10.1038/s41594-020-0438-0; Dong, et al., Journal of Medicinal Chemistry 64.15 (2021): 10606-10620, doi: 10.1021/acs.jmedchem.1c00895; Yang, et al., Targeted Oncology 16.1 (2021): 1-12, doi: 10.1007/s11523-020-00782-2; Lv, et al., Nature Communications 12.1 (2021): 6896, doi: 10.1038/s41467-021-27210-x.

### Compound Embodiments

Provided herein are compounds of Formula (I)

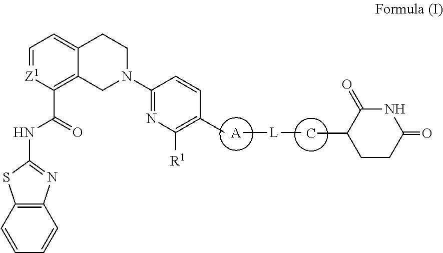

or pharmaceutically acceptable salts thereof, wherein:
Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
Ring A is selected from the group consisting of: (A1) and (A2):

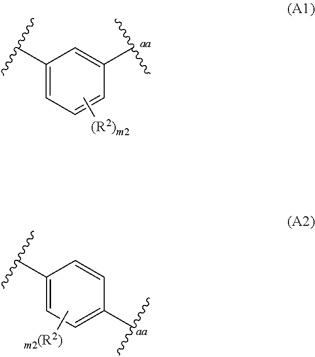

wherein:
m2 is 0, 1, or 2,
each R² is independently selected from the group consisting of: halo, —CN, C₁₋₃ alkoxy, C₁₋₃ haloalkoxy, and C₁₋₃ alkyl optionally substituted with 1-3 R^{c}, and
aa represents the point of attachment to L;
L is -(L^{A})ₙ₁-, wherein:
n1 is an integer from 4 to 10,
one L^{A} is L^{A4},
0-3 L^{A} are independently selected L^{A3}, and
each remaining L^{A} is an independently selected L^{A1};
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
each L^{A3} is independently selected from the group consisting of: —N(R^{d})—, —N(R^{h})—, —O—, —S(O)₀₋₂—, and —C(═O)—;
L^{A4} is independently selected from the group consisting of:
(a) C₃₋₁₂ cycloalkylene or 4-15 membered heterocyclylene, each of which is optionally substituted with 1-3 R^{L4}; and
(b) phenylene or 5-6 membered heteroarylene, each of which is optionally substituted with 1-3 R^{L4},
wherein each R^{L4} is independently selected from the group consisting of R^{a} and R^{b};
Ring C is selected from the group consisting of:

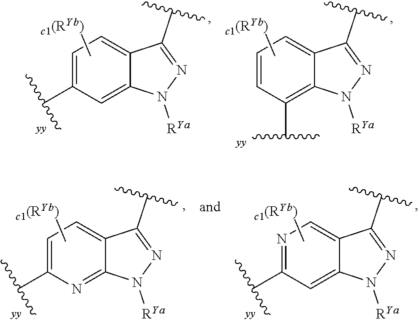

wherein:
R^{Ya} is selected from the group consisting of: —H, R^{b}, and C₁₋₆ alkyl optionally substituted with 1-3 R^{c};
each R^{Yb} is independently selected from the group consisting of R^{a} and R^{b};
c1 is 0, 1, or 2; and
yy represents the point of attachment to L;
each R^{a} is independently selected from the group consisting of:
(a) halo;
(b) —CN;
(c) —OH;
(d) oxo;
(e) C₁₋₆ alkoxy optionally substituted with 1-6 R^{c};
(f) —NR^{d}R^{e};
(g) —C(═O)C₁₋₆ alkyl;
(h) —C(═O)OC₁₋₆ alkyl;
(i) —C(═O)N(R^{f})₂;
(j) —S(O)₀₋₂(C₁₋₆ alkyl);
(k) —S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (I), Z¹ is N.

In some embodiments of Formula (I), Z¹ is CH.

In some embodiments of Formula (I), Ring A is (A1).

In some embodiments of Formula (I), Ring A is (A2).

In some embodiments of Formula (I), m2 is 1.

In some embodiments of Formula (I), Ring A is

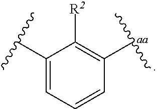

In some embodiments of Formula (I), Ring A is

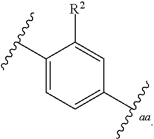

In some embodiments of Formula (I), Ring R² is C₁₋₃ alkyl optionally substituted with 1-3 R^{c} (e.g., C₁₋₃ alkyl optionally substituted with 1-3 F). For example, R² can be CH₃. For example, R² can be CF₃. In some embodiments of Formula (I), R² is —F.

In some embodiments of Formula (I), Ring A is:

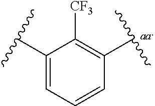 [SMILES: *c1cccc(*)c1C(F)(F)F]

In some embodiments of Formula (I), Ring A is:

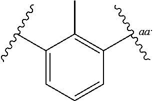 [SMILES: *c1cccc(*)c1C]

In some embodiments of Formula (I), Ring A is:

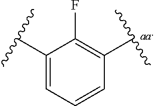 [SMILES: *c1cccc(*)c1F]

In some embodiments of Formula (I), Ring A is:

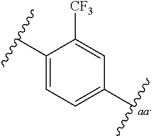 [SMILES: *c1ccc(*)c(C(F)(F)F)c1]

In some embodiments of Formula (I), Ring A is:

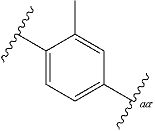 [SMILES: *c1ccc(*)c(C)c1]

In some embodiments of Formula (I), Ring A is:

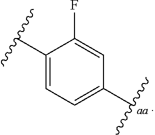 [SMILES: *c1ccc(*)c(F)c1]

In some embodiments of Formula (I), n1 is an integer from 5 to 9 (e.g., 5, 6, 7, or 8).

In some embodiments of Formula (I), n1 is an integer from 5 to 9; and L is:

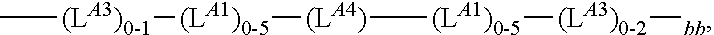

wherein bb represents the point of attachment to Ring C.

In some embodiments of Formula (I), n1 is an integer from 5 to 9; and L is selected from the group consisting of:

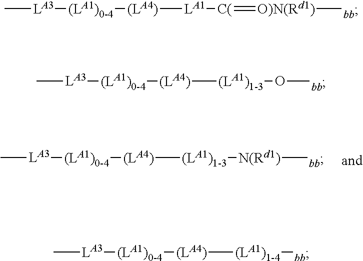

wherein bb represents the point of attachment to Ring C; and R^{d1} is H or C₁₋₃ alkyl.

In some embodiments of Formula (I), n1 is an integer from 5 to 9; and L is:

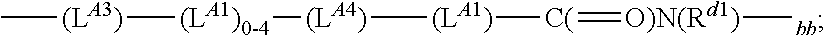

wherein bb represents the point of attachment to Ring C; and R^{d1} is H or C₁₋₃ alkyl.

In some embodiments of Formula (I), n1 is an integer from 5 to 9; and L is:

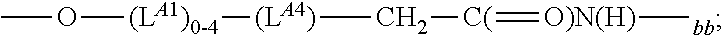

wherein bb represents the point of attachment to Ring C.

In some embodiments of Formula (I), n1 is an integer from 5 to 9; and L is:

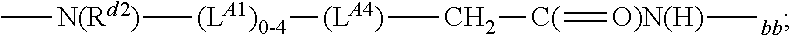

wherein bb represents the point of attachment to Ring C; and R^{d2} is H or C₁₋₃ alkyl. For example, R^{d2} can be H or methyl.

In some embodiments of Formula (I), L^{A4} is a 5-12 membered nitrogen-containing heterocyclylene optionally substituted with 1-3 R^{L4}.

In some embodiments of Formula (I), each R^{L4} is independently selected R^{a}.

In some embodiments of Formula (I), each R^{L4} is independently selected from the group consisting of: —F, CN, OH, C₁₋₃ alkoxy, and C₁₋₃ alkyl optionally substituted with 1-3 F.

In some embodiments of Formula (I), L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of Formula (I), L^{A4} is

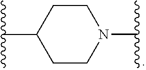 [SMILES: *C1CCN(*)CC1]

In some embodiments of Formula (I), L^{A4} is

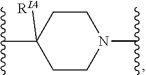

wherein R^{L4} is —F or —OH (e.g., —OH).

In some embodiments of Formula (I), L^{A4} is a bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of Formula (I), L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of Formula (I), L^{A4} is selected from the group consisting of:

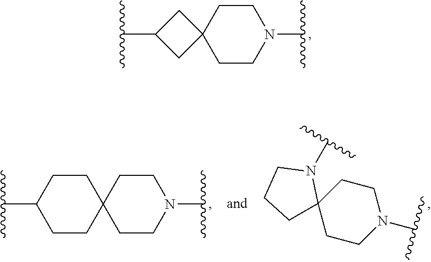 [SMILES: *C1CC2(CCN(*)CC2)C1 ; *C1CCC2(CC1)CCN(*)CC2 ; *N1CCC2(CCCN2*)CC1]

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F. For example, L^{A4} can be

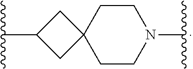 [SMILES: *C1CC2(CCN(*)CC2)C1]

In some embodiments of Formula (I), L^{A4} is a bridged or fused bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of Formula (I), L^{A4} is selected from the group consisting of:

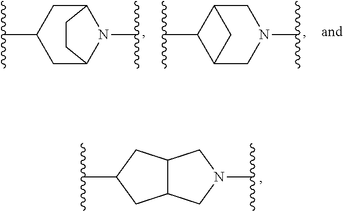

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of Formula (I), each L^{A1} is CH₂.

In some embodiments of Formula (I), one L^{A1} is CHR^{L} or C(R^{L})₂, wherein each R^{L} is independently —F or C₁₋₃ alkyl optionally substituted with 1-3 F; and each remaining L^{A1} is CH₂. For example, one L^{A1} can be CHMe; and each remaining L^{A1} can be CH₂.

In some embodiments of Formula (I), Ring C is

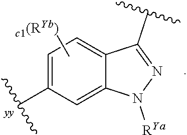

In some embodiments of Formula (I), Ring C is

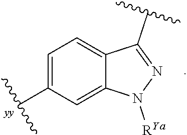

In some embodiments of Formula (I), Ring C is

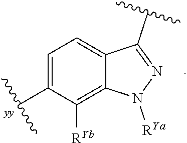

In some embodiments of Formula (I), Ring C is

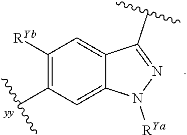

In some embodiments of Formula (I), R^{Ya} is selected from the group consisting of: H and C₁₋₆ alkyl optionally substituted with 1-3 R^{c}.

In some embodiments of Formula (I), R^{Ya} is C₁₋₃ alkyl optionally substituted with 1-3 R^{c} (e.g., C₁₋₃ alkyl optionally substituted with 1-3 F). For example, R^{Ya} can be methyl.

In some embodiments of Formula (I), each R^{Yb} is an independently selected R^{a}.

In some embodiments of Formula (I), each R^{Yb} is independently selected from the group consisting of: —F, C₁₋₃ alkoxy, and C₁₋₃ alkyl optionally substituted with 1-3 F.

In some embodiments of Formula (I), each R^{Yb} is independently selected from the group consisting of: —F, OMe, CH₃, CHF₂, CH₂F, and CF₃.

In some embodiments of Formula (I), at least one R^{Yb} is C₁₋₃ alkoxy (e.g., OMe).

In some embodiments of Formula (I), Ring C is

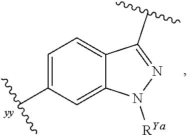

wherein R^{Ya} is C₁₋₃ alkyl optionally substituted with 1-3 R^{c} (e.g., C₁₋₃ alkyl optionally substituted with 1-3 F).

In some embodiments of Formula (I), Ring C is

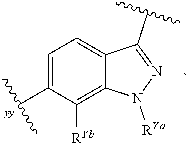

wherein R^{Ya} is C₁₋₃ alkyl optionally substituted with 1-3 R^{c} (e.g., C₁₋₃ alkyl optionally substituted with 1-3 F); and
R^{Yb} is independently selected from the group consisting of: —F, C₁₋₃ alkoxy, and C₁₋₃ alkyl optionally substituted with 1-3 F. In some embodiments when Ring C is

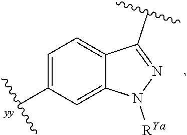

R^{Yb} is C₁₋₃ alkoxy (e.g., methoxy).

In some embodiments, the compounds of Formula (I) are compounds of Formula (I-1) or (I-2):

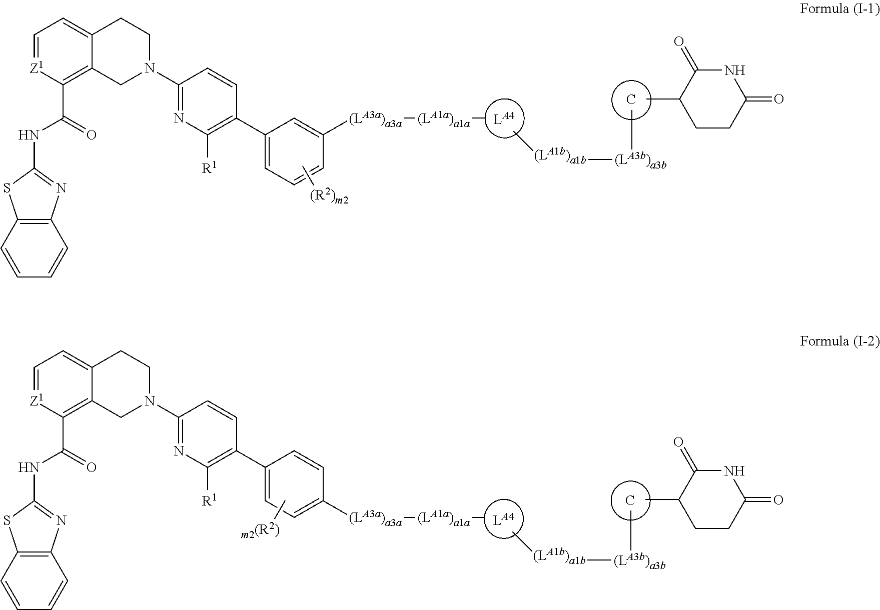

or pharmaceutically acceptable salts thereof, wherein:
m2 is 0 or 1;
each L^{A3a} and L^{A3b} is an independently selected L^{A3};
a3a is 0 or 1;
a3b is 0, 1, or 2;
each L^{A1a} and L^{A1b} is an independently selected L^{A1};
a1a and a1b are independently integers from 0 to 4;
provided that: a3a+a1a+a1b+a3b is from 4 to 8; and
L^{A4} is a 5-12 membered nitrogen-containing heterocyclylene optionally substituted with 1-3 R^{L4}.

In some embodiments, the compounds are compounds of Formula (I-1), or pharmaceutically acceptable salts thereof; and one or more of (1)-(4) applies:
(1) Z¹ is N;
(2) Ring C is

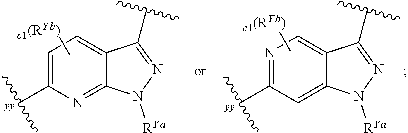

(3) c1 is 1 or 2, wherein at least one R^{Yb} is C₁₋₃ alkoxy (e.g., OMe); and
(4) a3a is 1, and L^{A3a} is —N(H)— or —N(C₁₋₃ alkyl)-.

In some embodiments, the compounds are compounds of Formula (I-2), or pharmaceutically acceptable salts thereof; and one or more of (1)-(5) applies:
(1) Z¹ is N;
(2) Ring C is

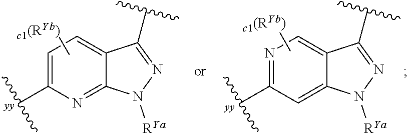

(3) c1 is 1 or 2, wherein at least one R^{Yb} is C₁₋₃ alkoxy;
(4) m2 is 1; and R² is CF₃; and
(5) a3a is 1, and L^{A3a} is —N(H)— or —N(C₁₋₃ alkyl)-.

In some embodiments, the compounds of Formula (I-1) or (I-2) are compounds of Formula (I-a1) or (I-a2):

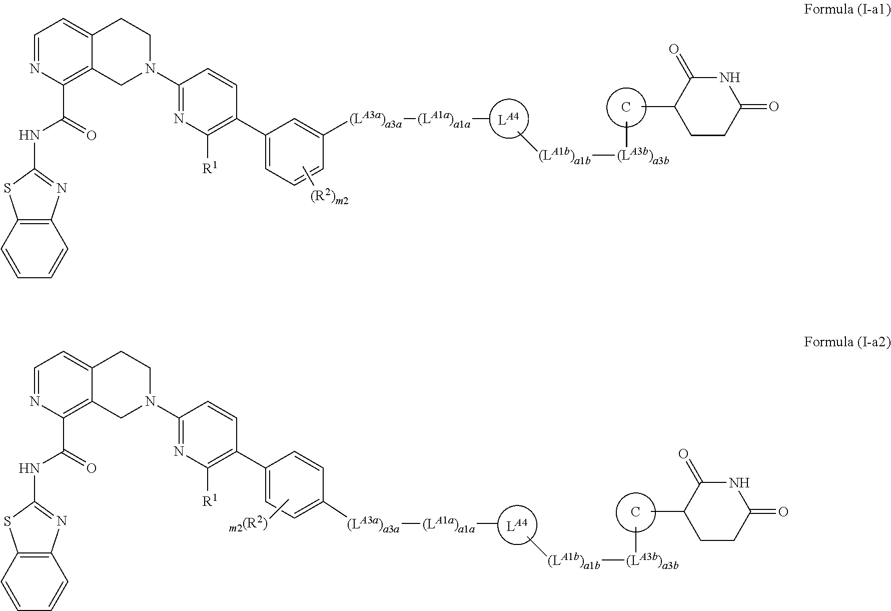

or pharmaceutically acceptable salts thereof.

In some embodiments, the compounds of Formula (I-1) or (I-2) are compounds of Formula (I-b1) or (I-b2):

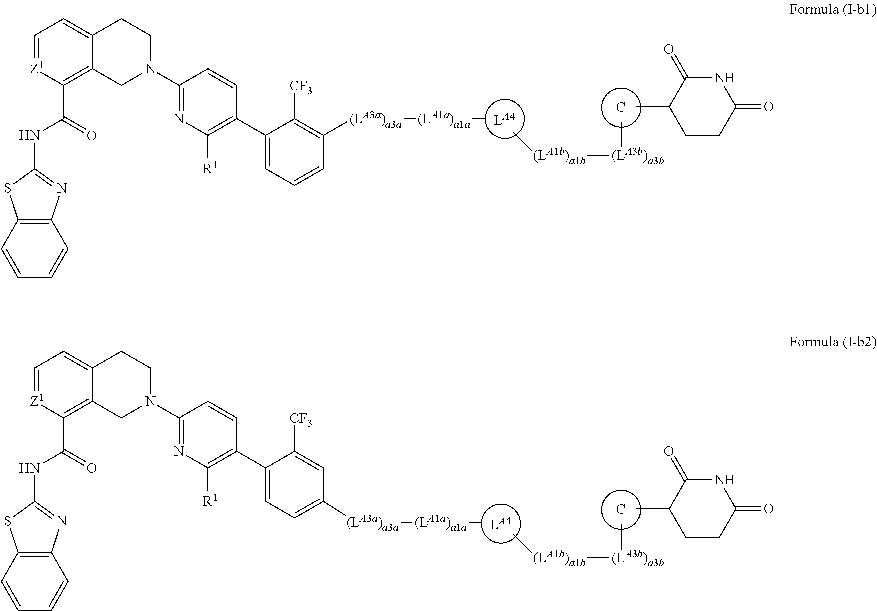

or pharmaceutically acceptable salts thereof.

In some embodiments, the compounds of Formula (I-1) or (I-2) are compounds of Formula (I-b3) or (I-b4):

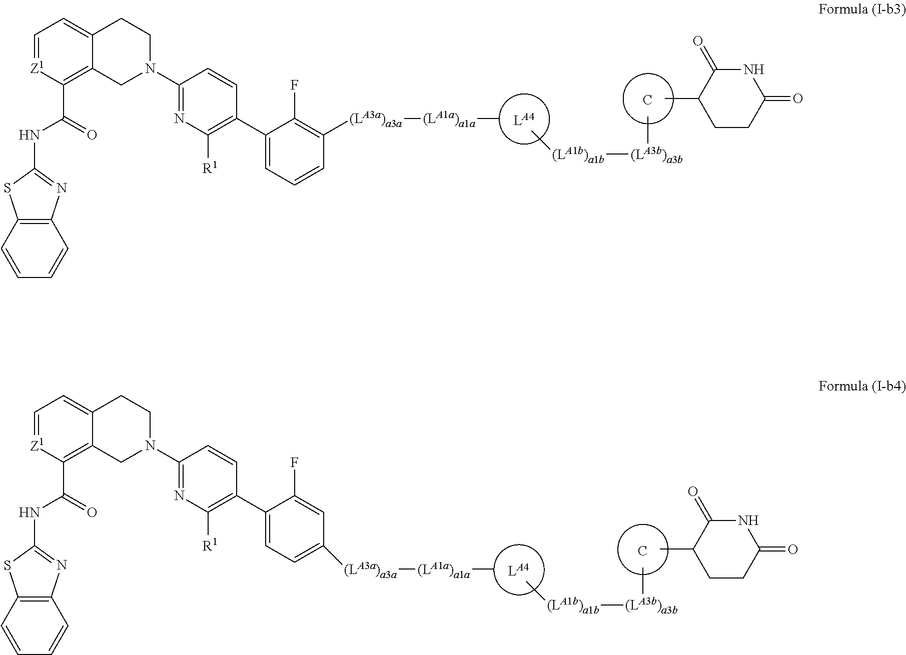

or pharmaceutically acceptable salts thereof.

In some embodiments of Formula (I-b1) or (I-b2), the compounds are other than Compound Nos. R126 or R313, as depicted in Table R1, or pharmaceutically acceptable salts thereof.

In some embodiments, the compounds are compounds of Formula (I-b2), or pharmaceutically acceptable salts thereof. In some embodiments, the compounds are compounds of Formula (I-b3), or pharmaceutically acceptable salts thereof. In some embodiments, the compounds are compounds of Formula (I-b4), or pharmaceutically acceptable salts thereof.

In some embodiments, the compounds are compounds of Formula (I-b1), or pharmaceutically acceptable salts thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and
(5) a1a is 0 or 1.

In some embodiments, the compounds of Formula (I-1) or (I-2) are compounds of Formula (I-c1) or (I-c2):

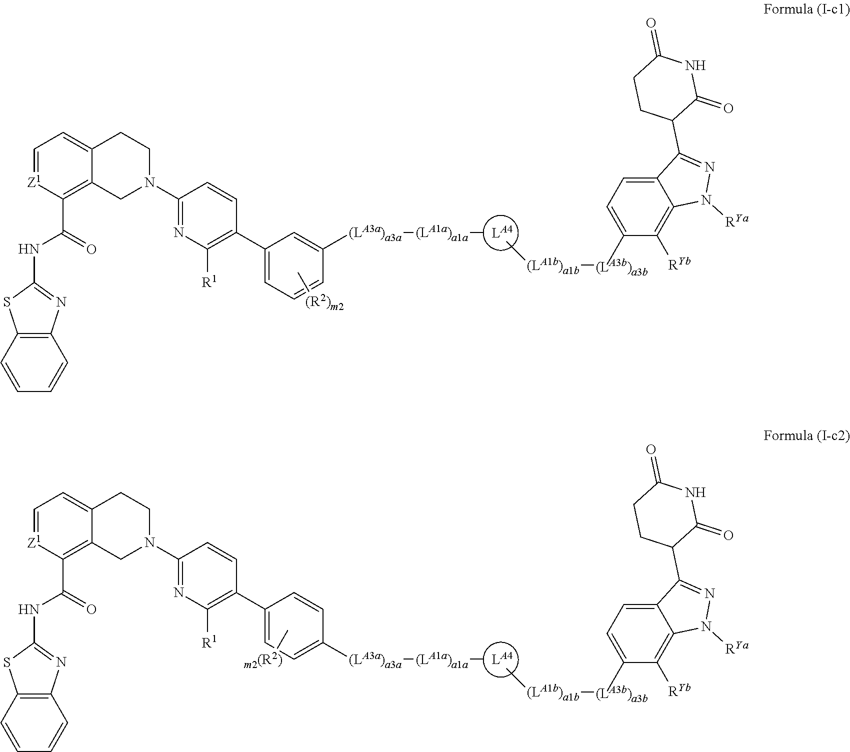

or pharmaceutically acceptable salts thereof, wherein:
R^{Yb} is F, C₁₋₃ alkoxy, or C₁₋₃ alkyl optionally substituted with 1-3 —F.

In some embodiments of Formula (I-c1) or (I-c2), the compounds are other than Compound Nos. R302 or R356, as depicted in Table R¹, or pharmaceutically acceptable salts thereof.

In some embodiments of Formula (I-c1) or (I-c2), R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4},
(4) at least one L^{A1a} is selected from the group consisting of CHR^{L} and C(R^{L})₂; and
(5) a1a is 0, 1, or 2.

In some embodiments of Formula (I-c1) or (I-c2), R^{Yb} is C₁₋₃ alkoxy (e.g., OMe).

In some embodiments, the compounds of Formula (I-1) or (I-2) are compounds of Formula (I-d1) or (I-d2):

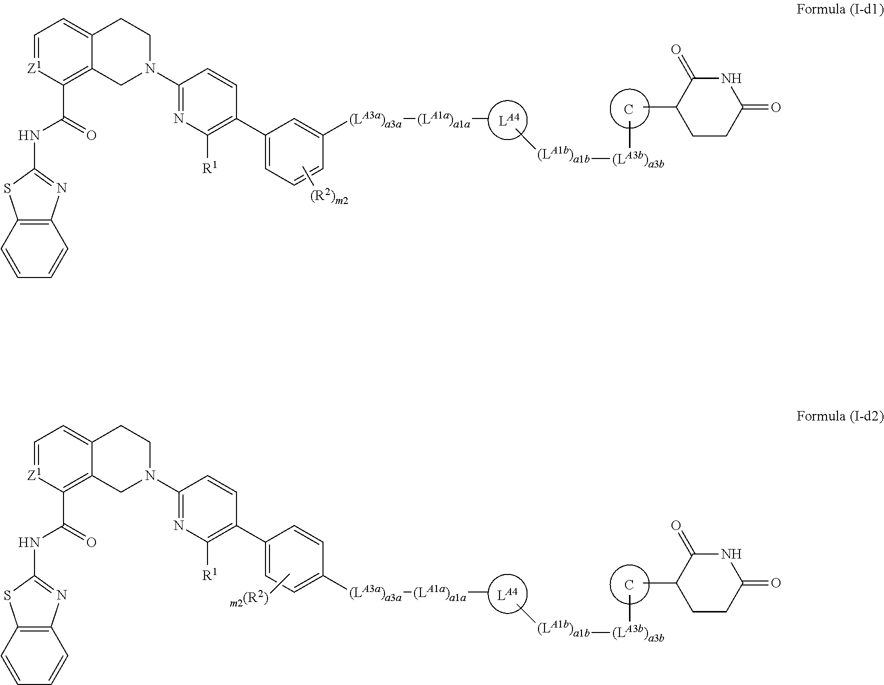

or pharmaceutically acceptable salts thereof, wherein:
L^{A4} is

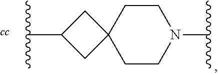

wherein cc represents point of attachment to -(L^{A1a})ₐ₁ₐ-.

In some embodiments of Formula (I-d1) or (I-d2), the compounds are other than Compound Nos. R156 or R226, as depicted in Table R1, or pharmaceutically acceptable salts thereof.

In some embodiments, the compounds are compounds of Formula (I-d1), or pharmaceutically acceptable salts thereof; and one of more of (1)-(5) applies:
(1) m2 is 1, and R² is CF₃;
(2) a1a is 0;
(3) a1a is 2 or 3;
(4) c1 is 1 or 2; and
(5) at least one L^{A1a} is selected from the group consisting of CHR^{L} and C(R^{L})₂.

In some embodiments, the compounds are compounds of Formula (I-d2), or pharmaceutically acceptable salts thereof.

In some embodiments of Formula (I-a1), (I-b1), (I-c1), (1-d1), (I-a2), (I-b2), (I-b3), (I-b4), (I-c2), (I-d2), a1b is 1; and L^{A1b} is —CH₂—.

In some embodiments of Formula (I-a1), (I-b1), (I-c1), (1-d1), (I-a2), (I-b2), (I-b3), (I-b4), (I-c2), (I-d2), a3a is 1; L^{A3a} is —O—; a3b is 2; and -(L^{A3b})_{a3b}- is —C(═O)NH— that is attached to Ring C at the nitrogen atom.

In some embodiments of Formula (I-a1), (I-b1), (I-c1), (1-d1), (I-a2), (I-b2), (I-b3), (I-b4), (I-c2), (I-d2), a3a is 1; L^{A3a} is —N(R^{d2})—, wherein R^{d2} is —H or C₁₋₃ alkyl; a3b is 2; and -(L^{A3b})_{a3b}- is —C(═O)NH— that is attached to Ring C at the nitrogen atom.

In some embodiments, the compounds of Formula (I-1) or (I-2) are compounds of Formula (I-e1) or (I-e2):

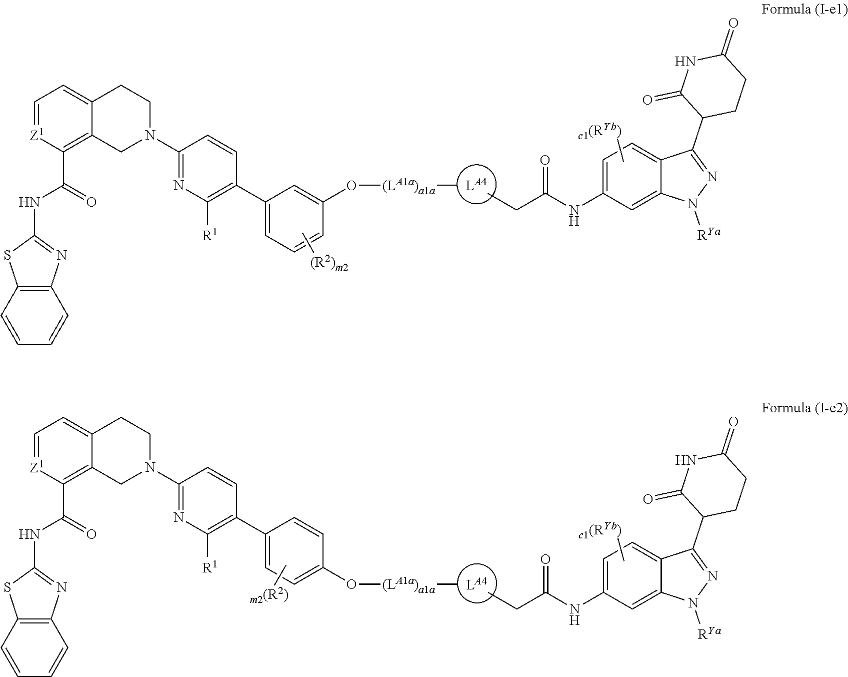

or pharmaceutically acceptable salts thereof, wherein:
a1a is an integer from 0 to 4;
m2 is 0 or 1; and
R² is CH₃ or CF₃.

In some embodiments, the compounds are compounds of Formula (I-e1), or pharmaceutically acceptable salts thereof; and one or more of (1)-(3) applies:
(1) Z¹ is N;
(2) Ring C is

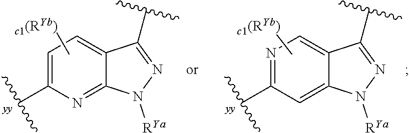

and
(3) c1 is 1 or 2, wherein at least one R^{Yb} is C₁₋₃ alkoxy.

In some embodiments, the compounds are compounds of Formula (I-e2), or pharmaceutically acceptable salts thereof; and one or more of (1)-(4) applies:
(1) Z¹ is N;
(2) Ring C is

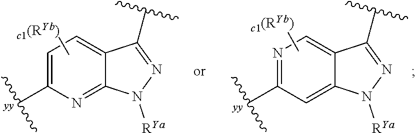

(3) c1 is 1 or 2, wherein at least one R^{Yb} is C₁₋₃ alkoxy; and
(4) m2 is 1; and R² is CF₃.

In some embodiments, the compounds of Formula (I-1) or (I-2) are compounds of Formula (I-e3) or (I-e4):

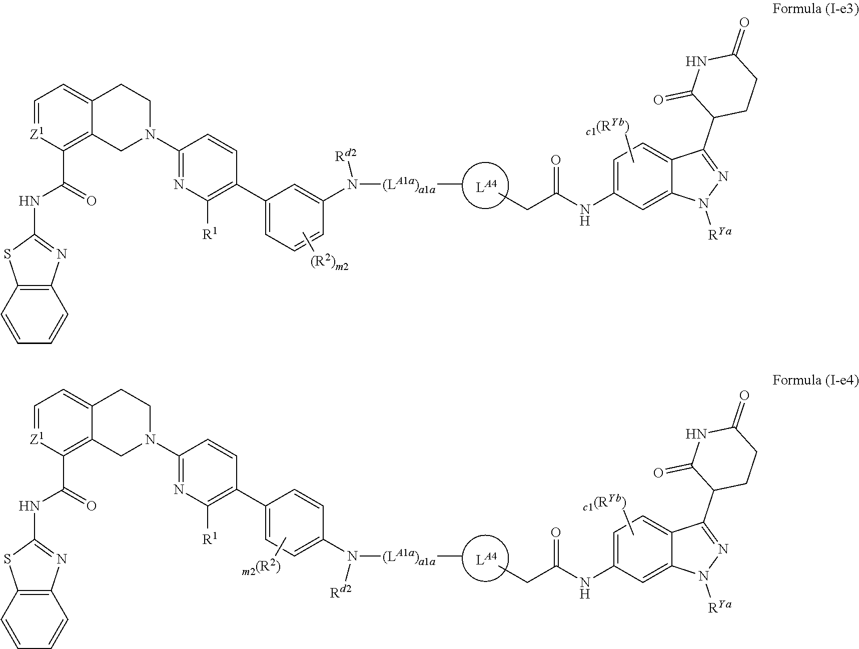

or a pharmaceutically acceptable salt thereof, wherein:
a1a is an integer from 0 to 4;
m2 is 0 or 1;
R² is CH₃, CF₃, or —F; and
R¹² is H or C₁₋₃ alkyl (e.g., H or methyl).

In some embodiments, the compounds of Formula (I-1) or (I-2) (e.g., Formula (I-a1) or (I-a2)) are compounds of Formula (I-f1) or (I-f2):

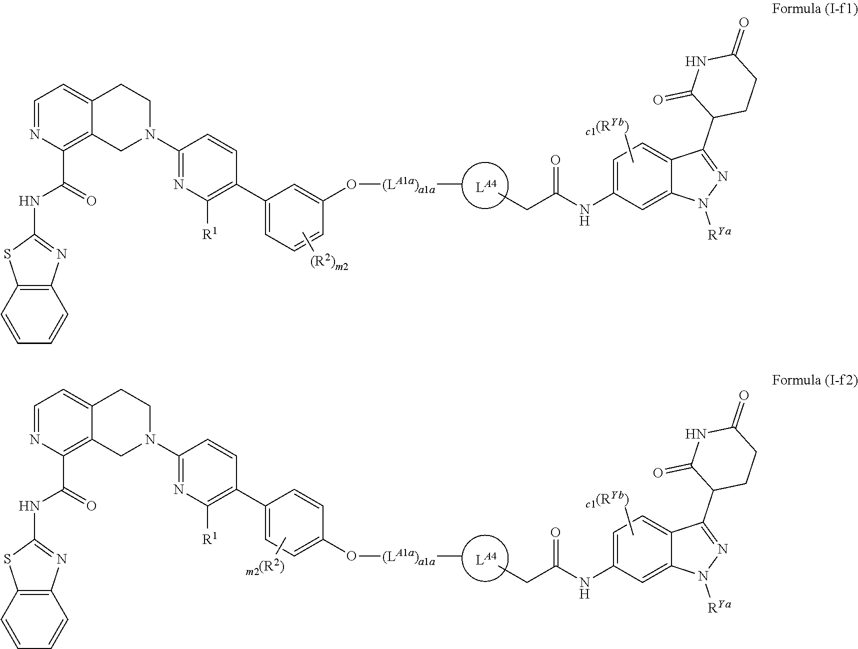

or pharmaceutically acceptable salts thereof, wherein:
a1a is an integer from 0 to 4;
m2 is 0 or 1; and
R² is CH₃ or CF₃.

In some embodiments, the compounds of Formula (I-1) or (I-2) (e.g., Formula (I-b1) or (I-b2)) are compounds of Formula (I-g1) or (I-g2):

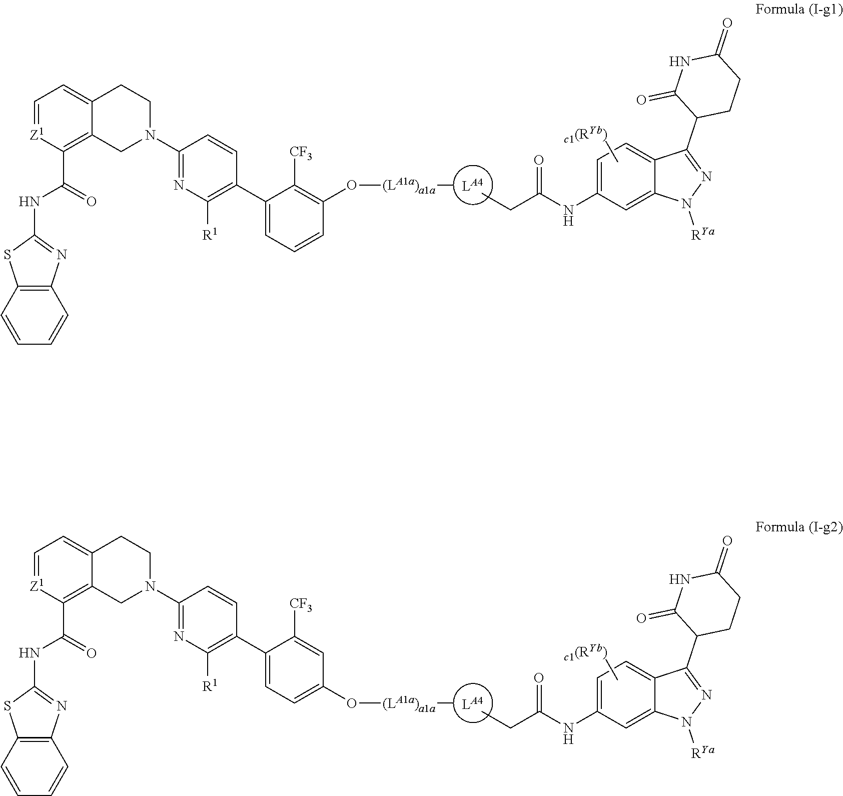

or pharmaceutically acceptable salts thereof, wherein:
a1a is an integer from 0 to 4;
m2 is 0 or 1; and
R² is CH₃ or CF₃.

In some embodiments of Formula (I-g1) or (I-g2), the compounds are other than Compound Nos. R126 or R313, as depicted in Table R¹, or pharmaceutically acceptable salts thereof.

In some embodiments, the compounds are compounds of Formula (I-g2), or pharmaceutically acceptable salts thereof.

In some embodiments, the compounds are compounds of Formula (I-g1), or pharmaceutically acceptable salts thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4}.
(4) at least one L^{A1a} is selected from the group consisting of CHR^{L} and C(R^{L})₂; and
(5) a1a is 0 or 1.

In some embodiments, the compounds of Formula (I-1) or (I-2) (e.g., Formula (I-b3) or (I-b4)) are compounds of Formula (I-g3) or (I-g4):

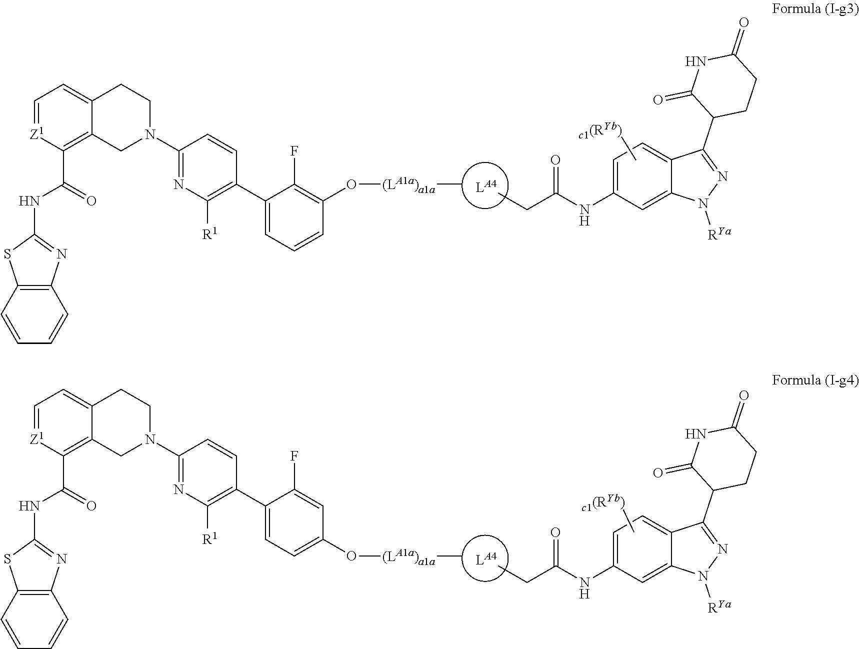

or a pharmaceutically acceptable salt thereof, wherein:
a1a is an integer from 0 to 4; and
m2 is 0 or 1.

In some embodiments, the compounds of Formula (I-1) or (I-2) (e.g., Formula (I-c1) or (I-c2)) are compounds of Formula (I-h1) or (I-h2):

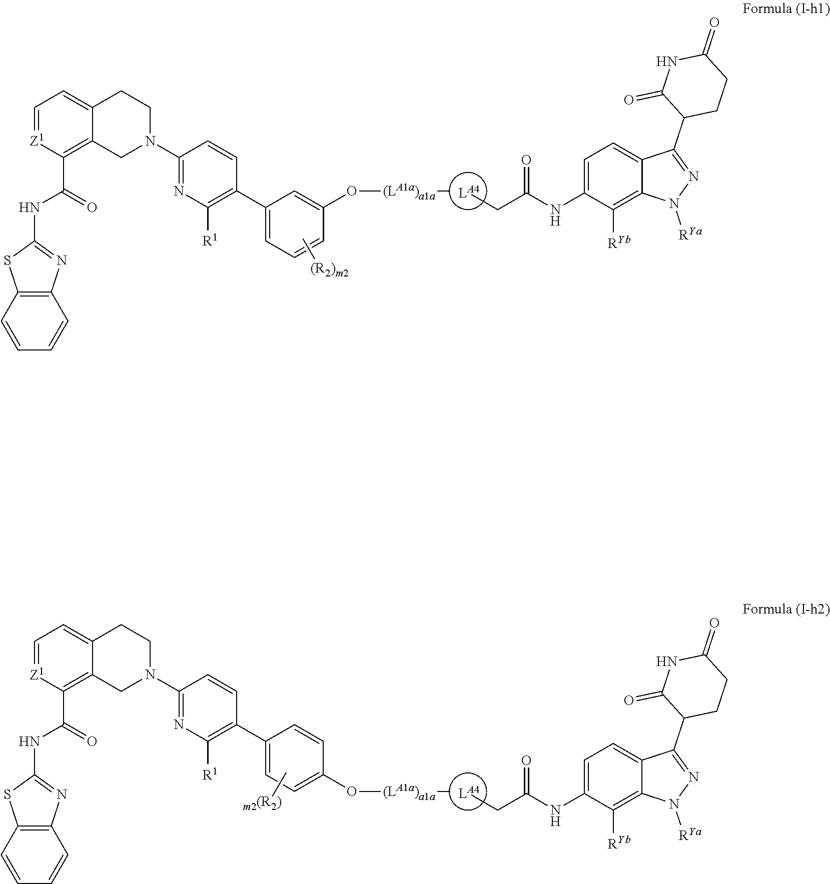

or pharmaceutically acceptable salts thereof, wherein:
a1a is an integer from 0 to 4;
m2 is 0 or 1;
R² is CH₃ or CF₃; and
R^{Yb} is F, C₁₋₃ alkoxy, or C₁₋₃ alkyl optionally substituted with 1-3 —F.

In some embodiments of Formula (I-h1) or (I-h2), the compounds are other than Compound Nos. R302 or R356, as depicted in Table R¹, or pharmaceutically acceptable salts thereof.

In some embodiments of Formula (I-h1) or (I-h2), R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4}.
(4) at least one L^{A1a} is selected from the group consisting of CHR^{L} and C(R^{L})₂; and
(5) a1a is 0, 1, or 2.

In some embodiments of Formula (I-h1) or (I-h2), R^{Yb} is C₁₋₃ alkoxy. For example, R^{Yb} can be OMe.

In some embodiments, the compounds of Formula (I-1) or (I-2) (e.g., Formula (I-d1) or (I-d2)) are compounds of Formula (I-i1) or (I-i2):

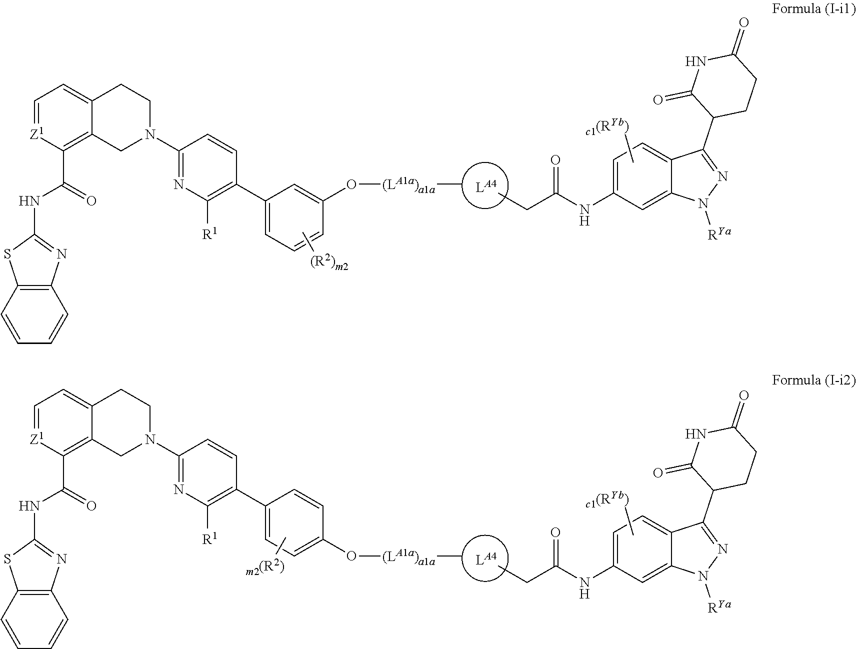

or pharmaceutically acceptable salts thereof, wherein:
a1a is an integer from 0 to 4;
L^{A4} is

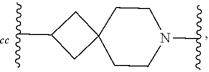

wherein cc represents point of attachment to -(L^{A1a})ₐ₁ₐ-.
m2 is 0 or 1; and
R² is CH₃ or CF₃.

In some embodiments of Formula (I-i1) or (I-i2), the compounds are other than Compound Nos. R156 or R226, as depicted in Table R¹, or pharmaceutically acceptable salts thereof.

In some embodiments, the compounds are compounds of Formula (I-i1), or pharmaceutically acceptable salts thereof; and one of more of (1)-(5) applies:
(1) m2 is 1, and R² is CF₃;
(2) a1a is 0;
(3) a1a is 2 or 3;
(4) c1 is 1 or 2; and
(5) at least one L^{A1a} is selected from the group consisting of CHR^{L} and C(R^{L})₂.

In some embodiments, the compounds are compounds of Formula (I-i2), or pharmaceutically acceptable salts thereof.

In some embodiments, the compounds of Formula (I-1) (e.g., Formula (I-d1)) are compounds of Formula (I-i3):

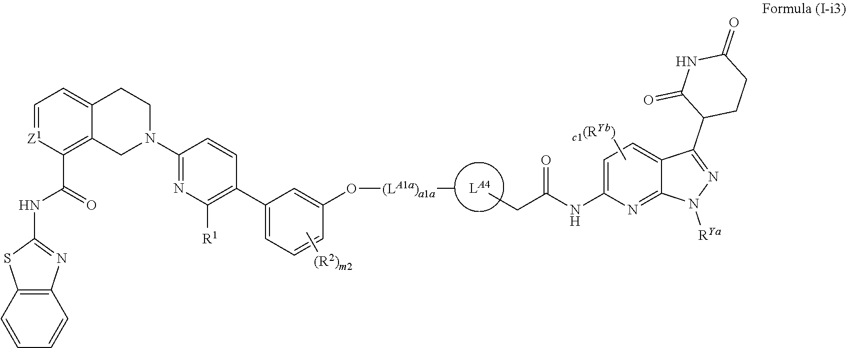

or pharmaceutically acceptable salts thereof, wherein:
a1a is an integer from 0 to 4;
L^{A4} is

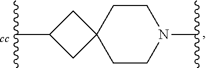

wherein cc represents point of attachment to -(L^{A1a})ₐ₁ₐ-.
m2 is 0 or; an
R² is CH₃ or CF₃.

In some embodiments of Formula (I-i3), c1 is 0. In some embodiments of Formula (I-i3), R^{Ya} is methyl.

In some embodiments of (I-a1), (I-b1), (I-b3), (I-c1), (I-d1), (I-e1), (I-e3), (I-f1), (I-g1), (I-g3), (I-h1), (I-i1), (I-i3), (I-a2), (I-b2), (I-b4), (I-c2), (I-d2), (I-e2), (I-e4), (I-f2), (I-g2), (I-g4), (I-h2), or (I-i2), a1a is 0 or 1.

In some embodiments of (I-a1), (I-b1), (I-b3), (I-c1), (I-d1), (I-e1), (I-e3), (I-f1), (I-g1), (I-g3), (I-h1), (I-i1), (I-i3), (I-a2), (I-b2), (I-b4), (I-c2), (I-d2), (I-e2), (I-e4), (I-f2), (I-g2), (I-g4), (I-h2), or (I-i2), a1a is 2, 3, or 4 (e.g., 2 or 3).

In some embodiments of (I-a1), (I-b1), (I-b3), (I-c1), (I-d1), (I-e1), (I-e3), (I-f1), (I-g1), (I-g3), (I-h1), (I-i1), (I-i3), (I-a2), (I-b2), (I-b4), (I-c2), (I-d2), (I-e2), (I-e4), (I-f2), (I-g2), (I-g4), (I-h2), or (I-i2), each L^{A1a} is CH₂.

In some embodiments of (I-a1), (I-b1), (I-b3), (I-c1), (I-d1), (I-e1), (I-e3), (I-f1), (I-g1), (I-g3), (I-h1), (I-i1), (I-i3), (I-a2), (I-b2), (I-b4), (I-c2), (I-d2), (I-e2), (I-e4), (I-f2), (I-g2), (I-g4), (I-h2), or (I-i2), one L^{A1a} is CHR^{L}, wherein R^{L} is —F or C₁₋₃ alkyl optionally substituted with 1-3 F; and each remaining L^{A1a} is CH₂. For example, one L^{A1a} can be CHMe; and each remaining L^{A1a} can be CH₂.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is

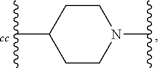

wherein cc represents the point of attachment to -(L^{A1a})ₐ₁ₐ-.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is

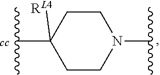

wherein R^{L4} is —F or —OH (e.g., —OH); and cc represents the point of attachment to -(L^{A1a})ₐ₁ₐ-.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is

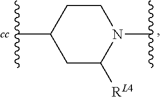

wherein R^{L4}, wherein R^{L4} is C₁₋₃ alkyl optionally substituted with 1-3 F; and cc represents the point of attachment to -(L^{A1a})ₐ₁ₐ-.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is selected from the group consisting of:

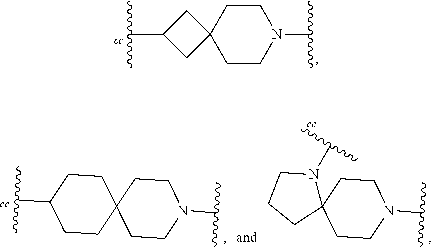 [SMILES: *C1CC2(CCN(*)CC2)C1 ; *C1CCC2(CC1)CCN(*)CC2 ; *N1CCC2(CCCN2*)CC1]

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F, and wherein cc represents the point of attachment to -(L^{A1a})ₐ₁ₐ-.

In some embodiments of (I-a1), (I-b1), (I-b3), (I-c1), (I-e1), (I-e3), (I-f1), (I-g1), (I-g3), (I-h1), (I-a2), (I-b2), (I-b4), (I-c2), (I-e2), (I-e4), (I-f2), (I-g2), (I-g4), or (I-h2), L^{A4} is

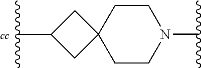

wherein cc represents the point of attachment to -(L^{A1a})ₐ₁ₐ-.

In some embodiments of (I-a1), (I-c1), (I-d1), (I-e1), (I-e3), (I-f1), (I-h1), (I-i1), (I-a2), (I-c2), (I-d2), (I-e2), (I-e4), (I-f2), (I-h2), or (I-i2), m2 is 1; and R² is CF₃.

In some embodiments of (I-a1), (I-c1), (I-d1), (I-e1), (I-e3), (I-f1), (I-h1), (I-i1), (I-a2), (I-c2), (I-d2), (I-e2), (I-e4), (I-f2), (I-h2), or (I-i2), m2 is 1; and R² is CF₃.

In some embodiments of (I-a1), (I-c1), (I-d1), (I-e1), (I-e3), (I-f1), (I-h1), (I-i1), (I-i3), (I-a2), (I-c2), (I-d2), (I-e2), (I-e4), (I-f2), (I-h2), or (I-i2), the

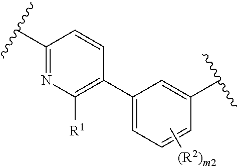

moiety is

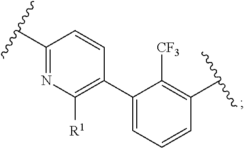

and
the

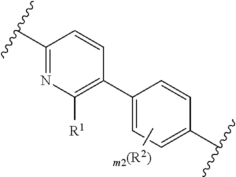

moiety is

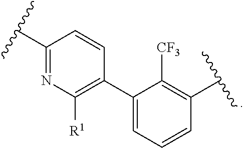

In some embodiments of (I-a1), (I-c1), (I-d1), (I-e1), (I-e3), (I-f1), (I-h1), (I-i1), (I-i3), (I-a2), (I-c2), (I-d2), (I-e2), (I-e4), (I-f2), (I-h2), or (I-i2), m2 is 1; and R² is CF₃.

In some embodiments of (I-a1), (I-c1), (I-d1), (I-e1), (I-e3), (I-f1), (I-h1), (I-i1), (I-i3), (I-a2), (I-c2), (I-d2), (I-e2), (I-e4), (I-f2), (I-h2), or (I-i2), the

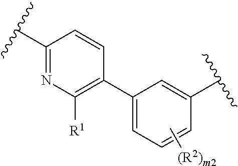

moiety is

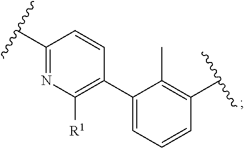

and
the

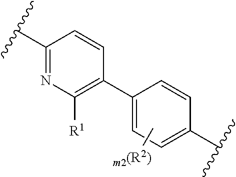

moiety is

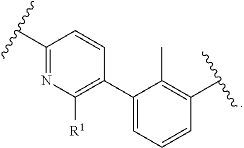

In some embodiments of (I-b1), (I-b3), (I-d1), (I-e1), (I-e3), (I-g1), (I-g3), (I-h1), (I-i1), (I-i3), (I-b2), (I-b4), (I-d2), (I-e2), (I-e4), (I-g2), (I-g4), (I-h2), or (I-i2), Z¹ is CH.

In some embodiments of (I-b1), (I-b3), (I-c1), (I-d1), (I-e1), (I-g1), (I-g3), (I-h1), (I-i1), (I-i3), (I-b2), (I-b4), (I-d2), (I-e2), (I-g2), (I-g4), (I-h2), or (I-i2), Z¹ is N.

In some embodiments of (I-a1), (I-b1), (I-b3), (I-d1), (I-e1), (I-g1), (I-g3), (I-h1), (I-i1), (I-i3), (I-a2), (I-b2), (I-b4), (I-d2), (I-e2), (I-g2), (I-g4), (I-h2), or (I-i2), each R^{Yb} is an independently selected R^{a}.

In some embodiments of (I-a1), (I-b1), (I-b3), (I-d1), (I-e1), (I-g1), (I-g3), (I-h1), (I-i1), (I-i3), (I-a2), (I-b2), (I-b4), (I-d2), (I-e2), (I-g2), (I-g4), (I-h2), or (I-i2), each R^{Yb} is independently selected from the group consisting of: —F, C₁₋₃ alkoxy, and C₁₋₃ alkyl optionally substituted with 1-3 F. For example, each R^{Yb} can be independently selected from the group consisting of: —F, OMe, CH₃, CHF₂, CH₂F, and CF₃.

In some embodiments of (I-a1), (I-b1), (I-b3), (I-d1), (I-e1), (I-g1), (I-g3), (I-i1), (I-i3), (I-a2), (I-b2), (I-b4), (I-d2), (I-e2), (I-g2), (I-g4), or (I-i2), c1 is 1. In some embodiments of (I-a1), (I-b1), (I-b3), (I-d1), (I-e1), (I-g1), (I-g3), (I-i1), (I-i3), (I-a2), (I-b2), (I-b4), (I-d2), (I-e2), (I-g2), (I-g4), or (I-i2), c1 is 0.

In some embodiments of (I-a1), (1-b1), (I-b3), (1-d1), (I-a2), (I-b2), (I-b4), or (I-d2), Ring C is

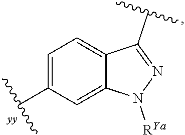

wherein R^{Ya} is C₁₋₃ alkyl optionally substituted with 1-3 R^{c}; and yy represents the point of attachment to -(L^{A3b})_{a3b}-. In some embodiments, R^{Ya} is methyl.

In some embodiments of (I-e1), (I-e3), (I-f1), (I-g1), (I-g3), (I-i1), (I-e2), (I-e4), (I-f2), (I-g2), (I-g4), or (I-i2), the

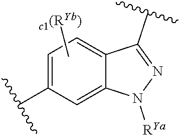

moiety is

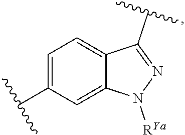

wherein R^{Ya} is C₁₋₃ alkyl optionally substituted with 1-3 R^{c}. In some embodiments, R^{Ya} is methyl.

In some embodiments of (I-a1), (1-b1), (I-b3), (1-d1), (I-a2), (I-b2), (I-b4), or (I-d2), Ring C is

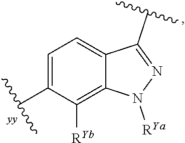

wherein R^{Ya} is C₁₋₃ alkyl optionally substituted with 1-3 R^{c};
R^{Yb} is selected from the group consisting of: —F, C₁₋₃ alkoxy, and C₁₋₃ alkyl optionally substituted with 1-3 F; and
yy represents the point of attachment to -(L^{A3b})_{a3b}-. In some embodiments, R^{Ya} is methyl. In some embodiments, R^{Yb} is C₁₋₃ alkoxy. In some embodiments, R^{Yb} is selected from the group consisting of: —F, CH₃, and OMe. For example, R^{Yb} can be OMe.

In some embodiments of (I-e1), (I-e3), (I-f1), (I-g1), (I-g3), (I-i1), (I-e2), (I-e4), (I-f2), (I-g2), (I-g4), or (I-i2), the

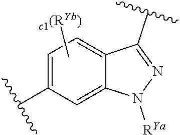

moiety is

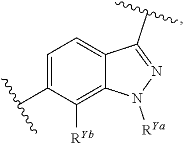

wherein R^{Ya} is C₁₋₃ alkyl optionally substituted with 1-3 R^{c}; and
R^{Yb} is selected from the group consisting of: —F, C₁₋₃ alkoxy, and C₁₋₃ alkyl optionally substituted with 1-3 F. In some embodiments, R^{Ya} is methyl. In some embodiments, R^{Yb} is C₁₋₃ alkoxy. In some embodiments, R^{Yb} is selected from the group consisting of: —F, CH₃, and OMe. For example, R^{Yb} can be OMe.

In some embodiments of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), R¹ is C(O)OH.

In some embodiments, the compounds of Formula (I) (e.g., Formula (I-1) (e.g., (I-a1), (I-b1), (I-b3), (I-c1), (I-d1), (I-e1), (I-e3), (I-41), (1-g1), (I-g3), (I-h1), (I-i1), or (I-i3)) or Formula (I-2) (e.g., (I-a2), (I-b2), (I-b4), (I-c2), (I-d2), (I-e2), (I-e4), (I-42), (I-g2), (I-g4), I-h2), or (I-i2))) are selected from the group consisting of the compounds depicted in Table C1, or pharmaceutically acceptable salts thereof.

TABLE C1

| No. | Compound Structure |
|---|---|
| 101 | 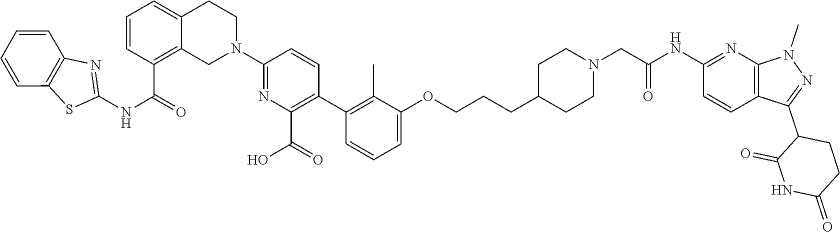 [SMILES: Cc1c(OCCCC2CCN(CC(=O)Nc3ccc4c(C5CCC(=O)NC5=O)nn(C)c4n3)CC2)cccc1-c1ccc(N2CCc3cccc(C(=O)Nc4nc5ccccc5s4)c3C2)nc1C(=O)O] |
| 103 | 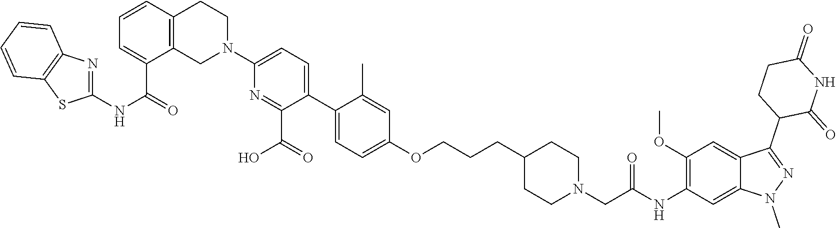 [SMILES: COc1cc2c(C3CCC(=O)NC3=O)nn(C)c2cc1NC(=O)CN1CCC(CCCOc2ccc(-c3ccc(N4CCc5cccc(C(=O)Nc6nc7ccccc7s6)c5C4)nc3C(=O)O)c(C)c2)CC1] |
| 104 | 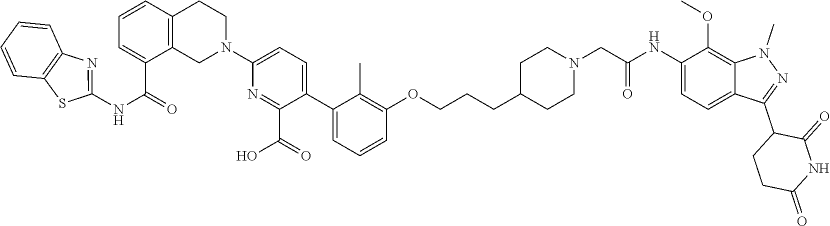 [SMILES: COc1c(NC(=O)CN2CCC(CCCOc3cccc(-c4ccc(N5CCc6cccc(C(=O)Nc7nc8ccccc8s7)c6C5)nc4C(=O)O)c3C)CC2)ccc2c(C3CCC(=O)NC3=O)nn(C)c12] |
| 105 | 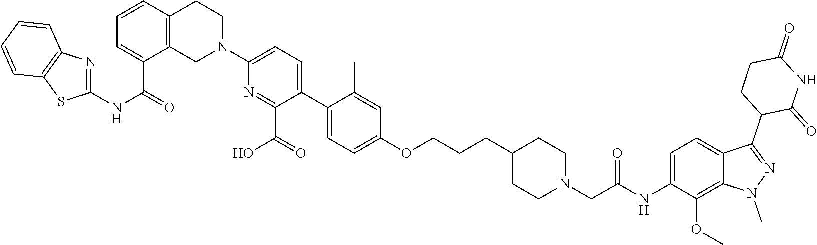 [SMILES: COc1c(NC(=O)CN2CCC(CCCOc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| 106 | 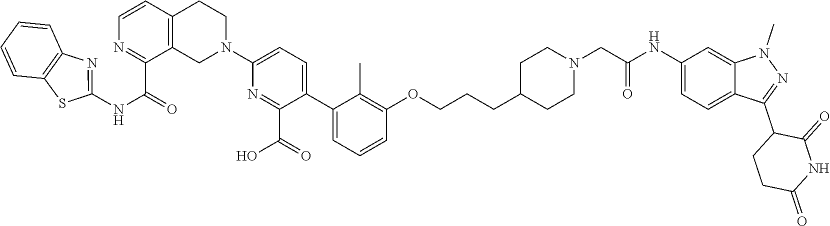 [SMILES: Cc1c(OCCCC2CCN(CC(=O)Nc3ccc4c(C5CCC(=O)NC5=O)nn(C)c4c3)CC2)cccc1-c1ccc(N2CCc3ccnc(C(=O)Nc4nc5ccccc5s4)c3C2)nc1C(=O)O] |
| 107 | 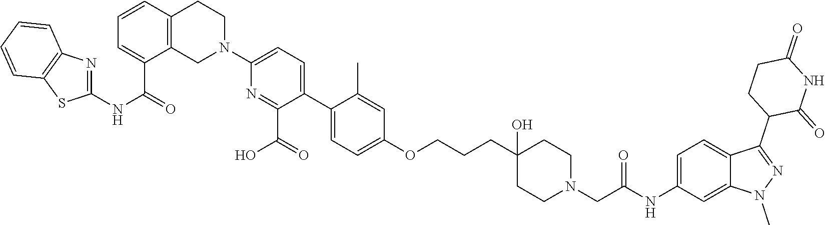 [SMILES: Cc1cc(OCCCC2(O)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 108 | 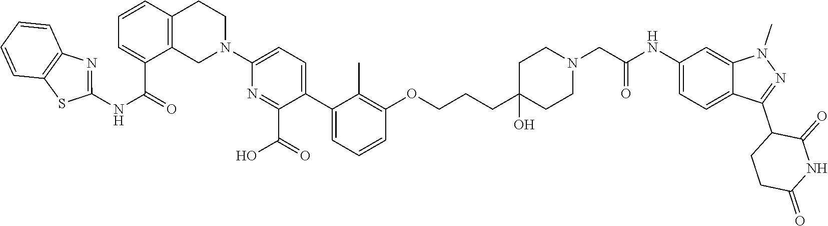 [SMILES: Cc1c(OCCCC2(O)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 109 | 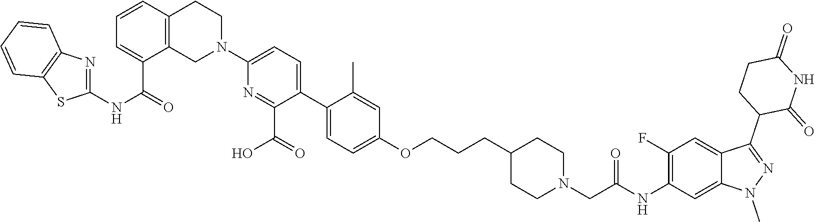 [SMILES: Cc1cc(OCCCC2CCN(CC(=O)Nc3cc4c(cc3F)c(C3CCC(=O)NC3=O)nn4C)CC2)ccc1-c1ccc(N2CCc3cccc(C(=O)Nc4nc5ccccc5s4)c3C2)nc1C(=O)O] |
| 112 | 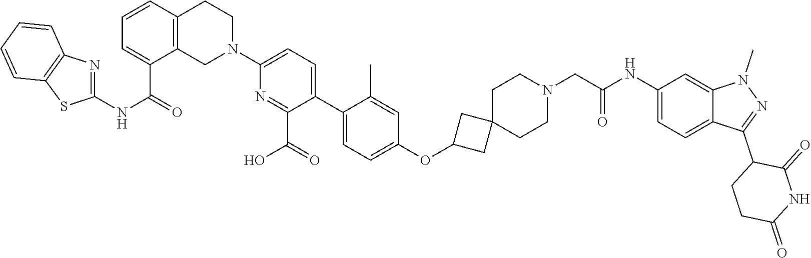 [SMILES: Cc1cc(OC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O] |
| 113 | 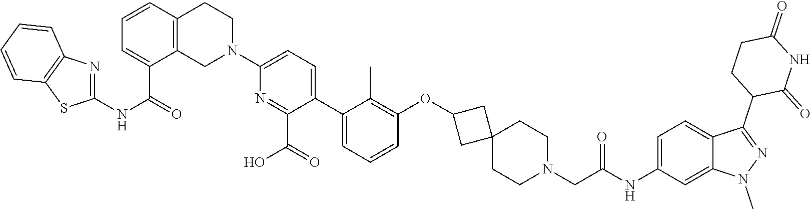 [SMILES: Cc1c(OC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 114 | 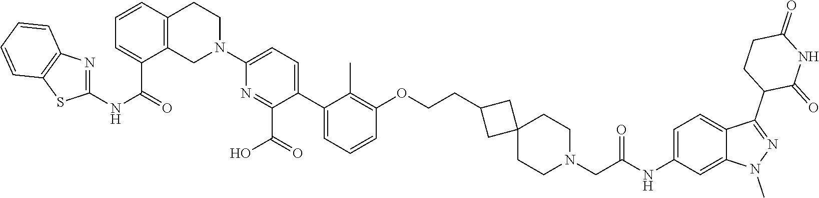 [SMILES: Cc1c(OCCC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 115 | 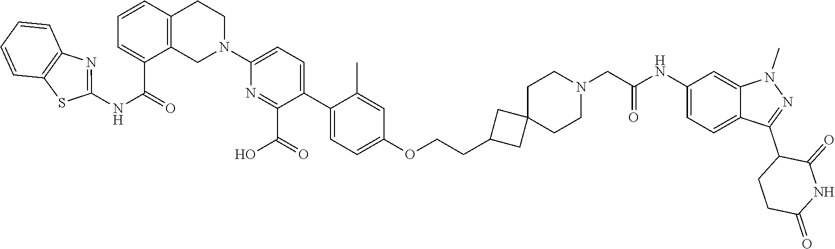 [SMILES: Cc1cc(OCCC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O] |
| 116 | 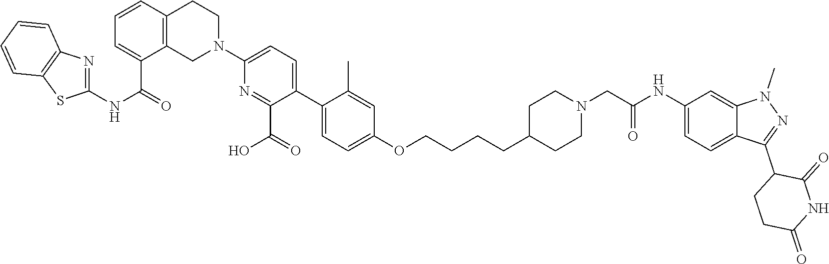 [SMILES: Cc1cc(OCCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 117 | 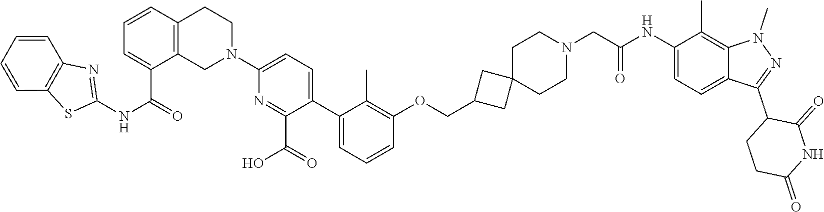 [SMILES: Cc1c(OCC2CC3(CCN(CC(=O)Nc4ccc5c(C6CCC(=O)NC6=O)nn(C)c5c4C)CC3)C2)cccc1-c1ccc(N2CCc3cccc(C(=O)Nc4nc5ccccc5s4)c3C2)nc1C(=O)O] |
| 118 | 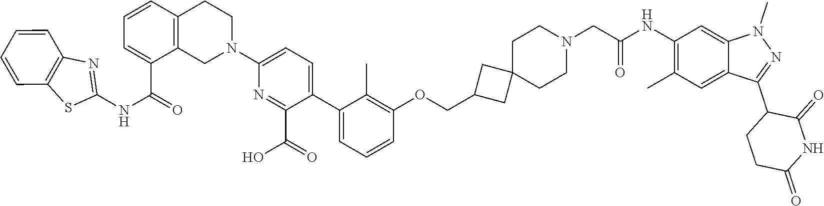 [SMILES: Cc1cc2c(C3CCC(=O)NC3=O)nn(C)c2cc1NC(=O)CN1CCC2(CC1)CC(COc1cccc(-c3ccc(N4CCc5cccc(C(=O)Nc6nc7ccccc7s6)c5C4)nc3C(=O)O)c1C)C2] |
| 119 | 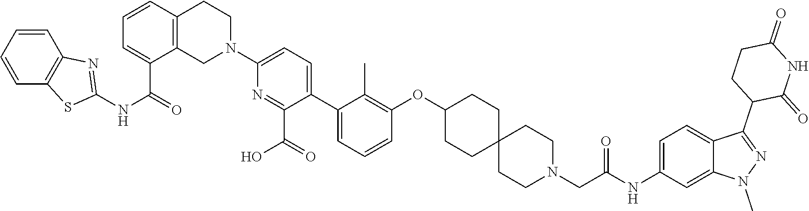 [SMILES: Cc1c(OC2CCC3(CC2)CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 120 | 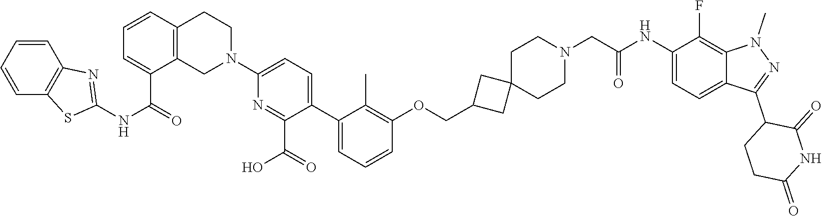 [SMILES: Cc1c(OCC2CC3(CCN(CC(=O)Nc4ccc5c(C6CCC(=O)NC6=O)nn(C)c5c4F)CC3)C2)cccc1-c1ccc(N2CCc3cccc(C(=O)Nc4nc5ccccc5s4)c3C2)nc1C(=O)O] |
| 121 | 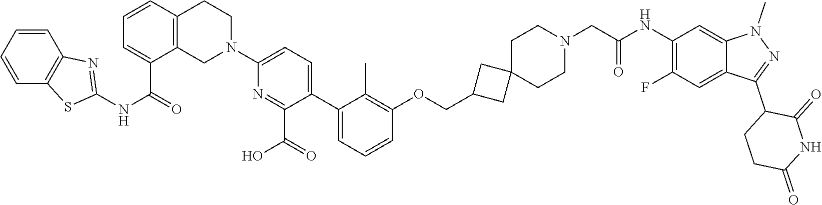 [SMILES: Cc1c(OCC2CC3(CCN(CC(=O)Nc4cc5c(cc4F)c(C4CCC(=O)NC4=O)nn5C)CC3)C2)cccc1-c1ccc(N2CCc3cccc(C(=O)Nc4nc5ccccc5s4)c3C2)nc1C(=O)O] |
| 122 | 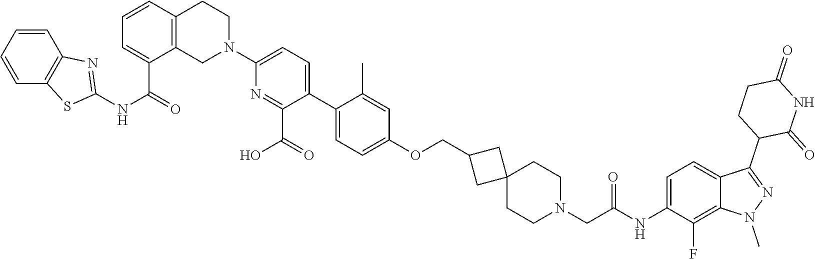 [SMILES: Cc1cc(OCC2CC3(CCN(CC(=O)Nc4ccc5c(C6CCC(=O)NC6=O)nn(C)c5c4F)CC3)C2)ccc1-c1ccc(N2CCc3cccc(C(=O)Nc4nc5ccccc5s4)c3C2)nc1C(=O)O] |
| 123 | 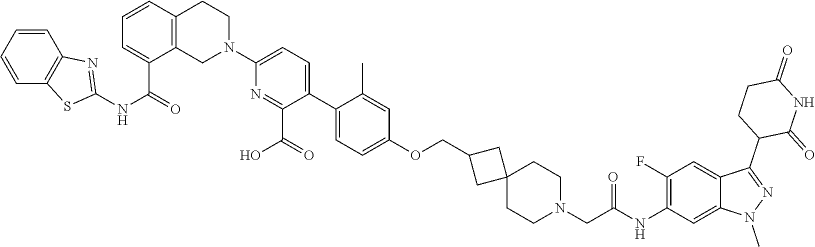 [SMILES: Cc1cc(OCC2CC3(CCN(CC(=O)Nc4cc5c(cc4F)c(C4CCC(=O)NC4=O)nn5C)CC3)C2)ccc1-c1ccc(N2CCc3cccc(C(=O)Nc4nc5ccccc5s4)c3C2)nc1C(=O)O] |
| 124 | 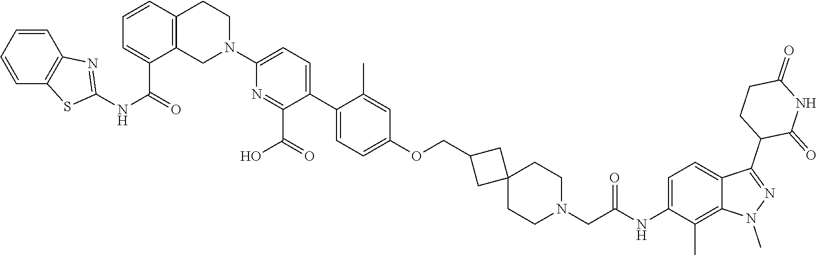 [SMILES: Cc1cc(OCC2CC3(CCN(CC(=O)Nc4ccc5c(C6CCC(=O)NC6=O)nn(C)c5c4C)CC3)C2)ccc1-c1ccc(N2CCc3cccc(C(=O)Nc4nc5ccccc5s4)c3C2)nc1C(=O)O] |
| 125 | 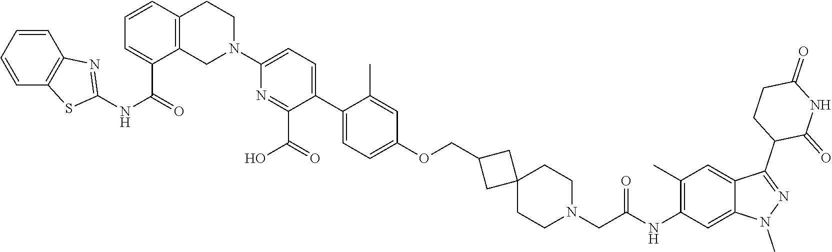 [SMILES: Cc1cc2c(C3CCC(=O)NC3=O)nn(C)c2cc1NC(=O)CN1CCC2(CC1)CC(COc1ccc(-c3ccc(N4CCc5cccc(C(=O)Nc6nc7ccccc7s6)c5C4)nc3C(=O)O)c(C)c1)C2] |
| 126 | 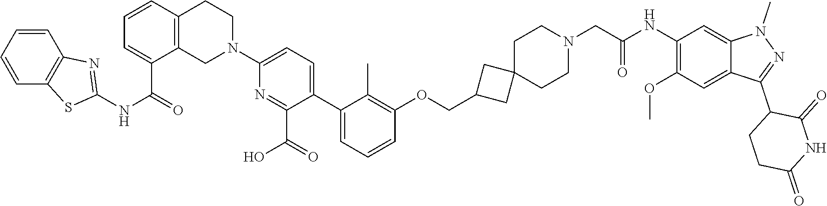 [SMILES: COc1cc2c(C3CCC(=O)NC3=O)nn(C)c2cc1NC(=O)CN1CCC2(CC1)CC(COc1cccc(-c3ccc(N4CCc5cccc(C(=O)Nc6nc7ccccc7s6)c5C4)nc3C(=O)O)c1C)C2] |
| 127 | 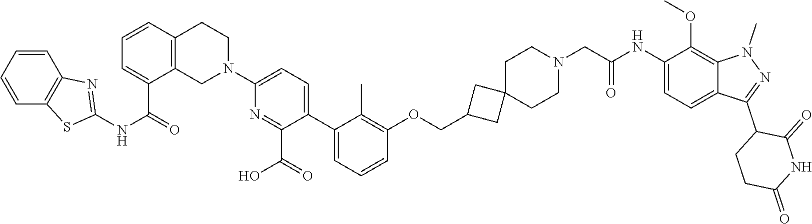 [SMILES: COc1c(NC(=O)CN2CCC3(CC2)CC(COc2cccc(-c4ccc(N5CCc6cccc(C(=O)Nc7nc8ccccc8s7)c6C5)nc4C(=O)O)c2C)C3)ccc2c(C3CCC(=O)NC3=O)nn(C)c12] |
| 128 | 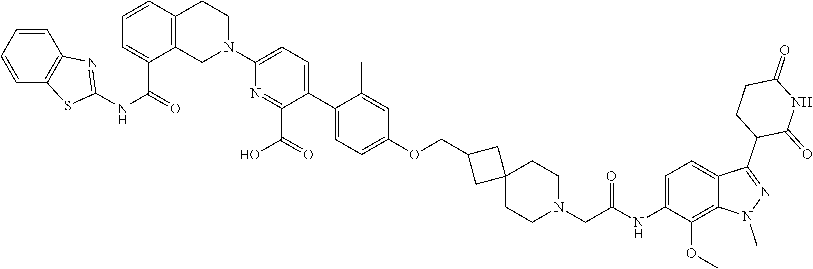 [SMILES: COc1c(NC(=O)CN2CCC3(CC2)CC(COc2ccc(-c4ccc(N5CCc6cccc(C(=O)Nc7nc8ccccc8s7)c6C5)nc4C(=O)O)c(C)c2)C3)ccc2c(C3CCC(=O)NC3=O)nn(C)c12] |
| 129 | 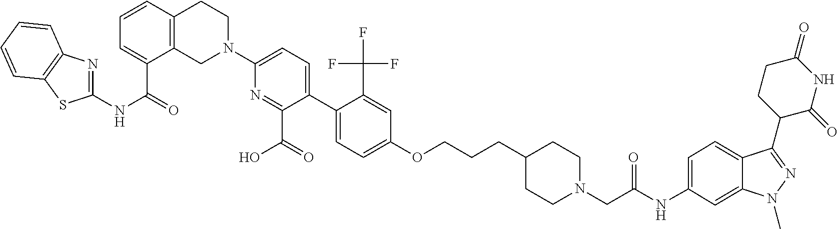 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(C(F)(F)F)c4)CC3)cc21] |
| 130 | 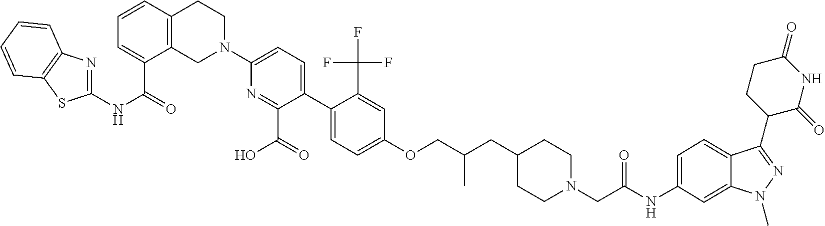 [SMILES: CC(COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| 130a | 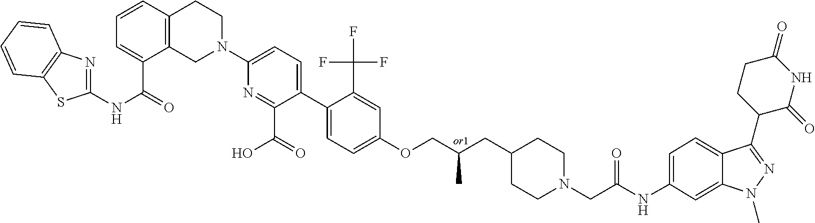 [SMILES: C[C@@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| 130b | 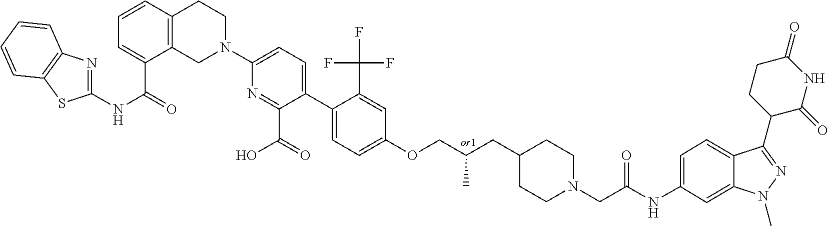 [SMILES: C[C@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| 131 | 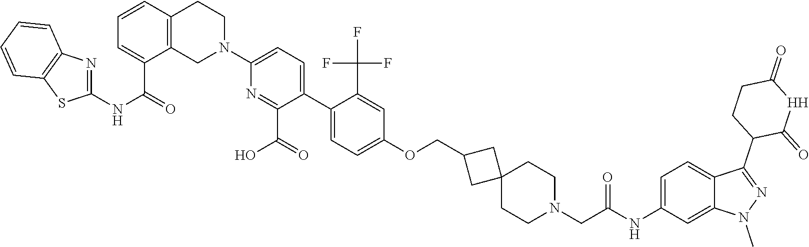 |
| 132 | 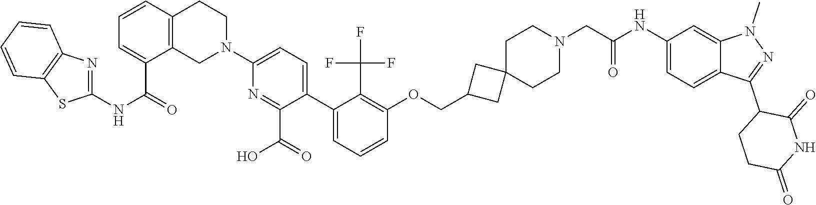 [SMILES: Cn1nc(C2CCC(=O)NC2=O)c2ccc(NC(=O)CN3CCC4(CC3)CC(COc3cccc(-c5ccc(N6CCc7cccc(C(=O)Nc8nc9ccccc9s8)c7C6)nc5C(=O)O)c3C(F)(F)F)C4)cc21] |
| 133 | 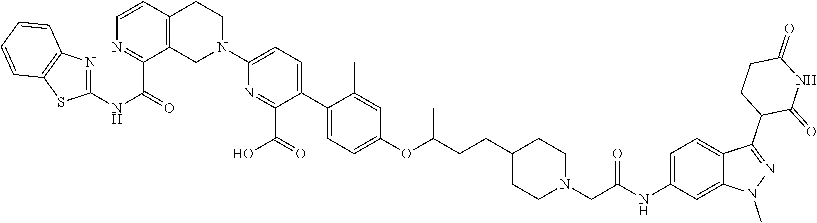 [SMILES: Cc1cc(OC(C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 133a | 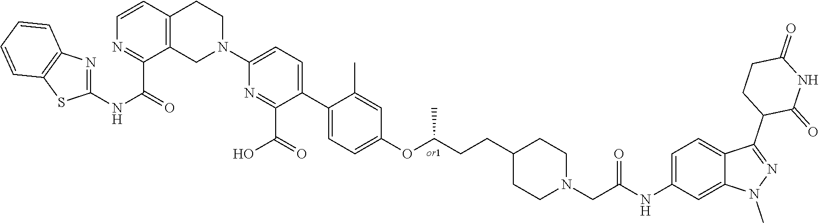 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 133b | 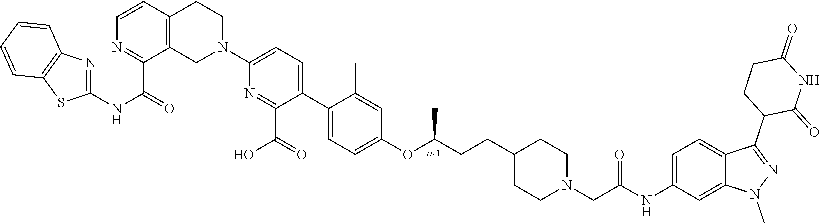 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 134 | 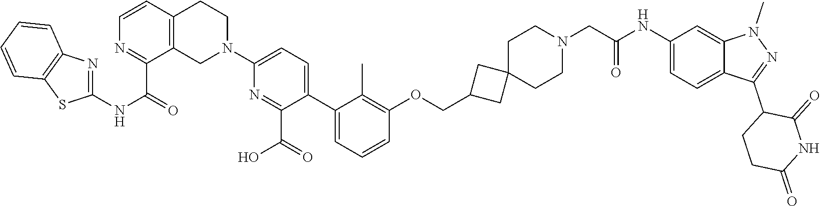 [SMILES: Cc1c(OCC2CC3(CCN(CC(=O)Nc4ccc5c(C6CCC(=O)NC6=O)nn(C)c5c4)CC3)C2)cccc1-c1ccc(N2CCc3ccnc(C(=O)Nc4nc5ccccc5s4)c3C2)nc1C(=O)O] |
| 135 | 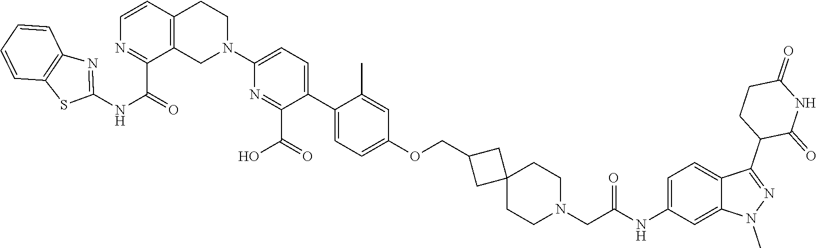 [SMILES: Cc1cc(OCC2CC3(CCN(CC(=O)Nc4ccc5c(C6CCC(=O)NC6=O)nn(C)c5c4)CC3)C2)ccc1-c1ccc(N2CCc3ccnc(C(=O)Nc4nc5ccccc5s4)c3C2)nc1C(=O)O] |
| 136 | 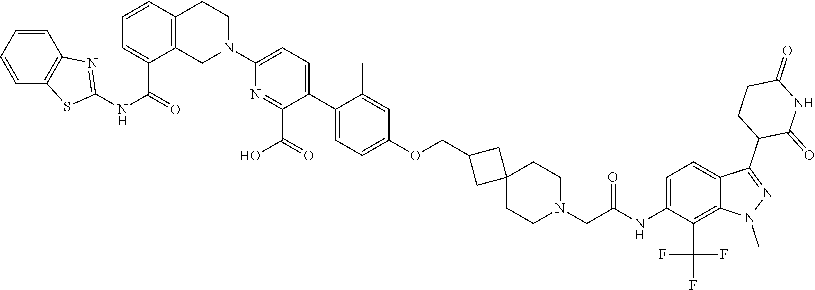 [SMILES: Cc1cc(OCC2CC3(CCN(CC(=O)Nc4ccc5c(C6CCC(=O)NC6=O)nn(C)c5c4C(F)(F)F)CC3)C2)ccc1-c1ccc(N2CCc3cccc(C(=O)Nc4nc5ccccc5s4)c3C2)nc1C(=O)O] |
| 137 | 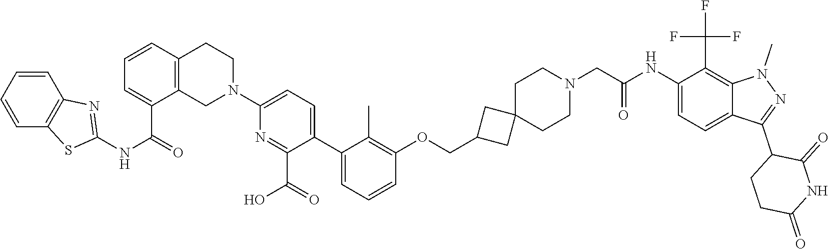 [SMILES: Cc1c(OCC2CC3(CCN(CC(=O)Nc4ccc5c(C6CCC(=O)NC6=O)nn(C)c5c4C(F)(F)F)CC3)C2)cccc1-c1ccc(N2CCc3cccc(C(=O)Nc4nc5ccccc5s4)c3C2)nc1C(=O)O] |
| 138 | 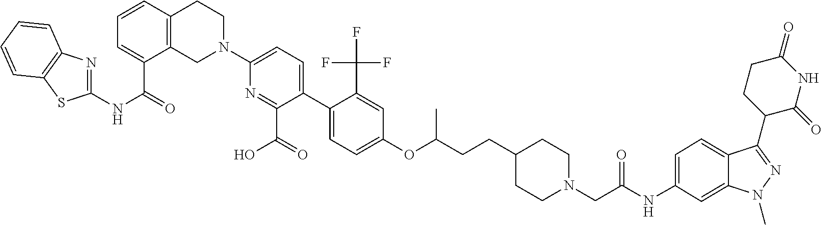 [SMILES: CC(CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| 138a | 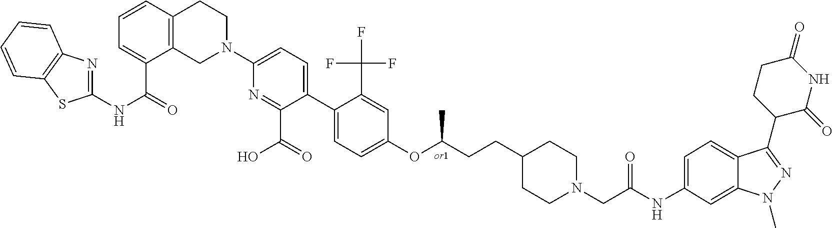 [SMILES: C[C@@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| 138b | 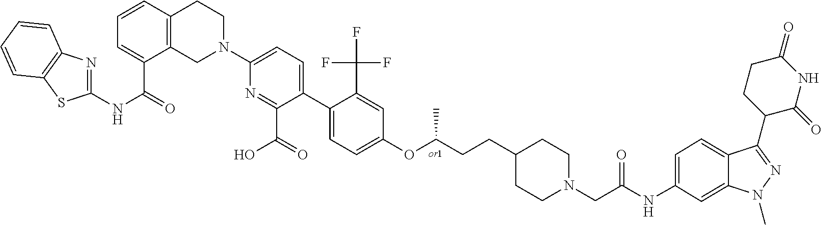 [SMILES: C[C@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| 139 | 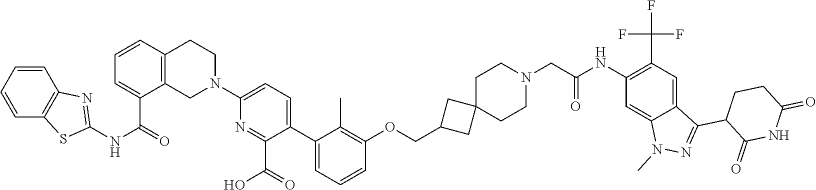 [SMILES: Cc1c(OCC2CC3(CCN(CC(=O)Nc4cc5c(cc4C(F)(F)F)c(C4CCC(=O)NC4=O)nn5C)CC3)C2)cccc1-c1ccc(N2CCc3cccc(C(=O)Nc4nc5ccccc5s4)c3C2)nc1C(=O)O] |
| 140 | 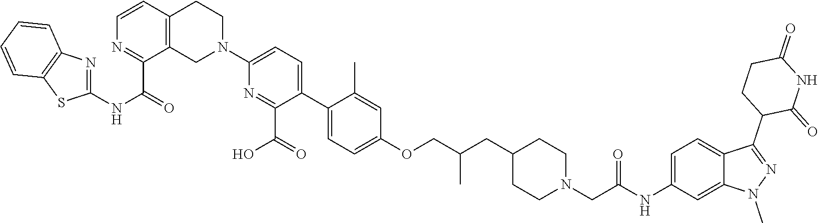 [SMILES: Cc1cc(OCC(C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 140a | 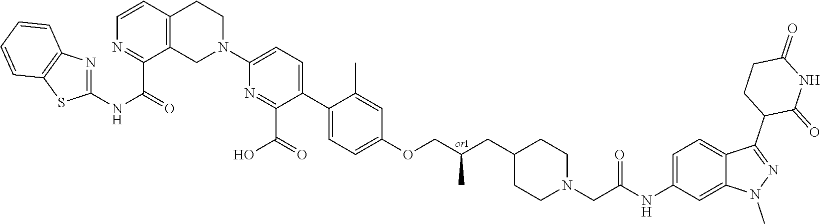 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 140b | 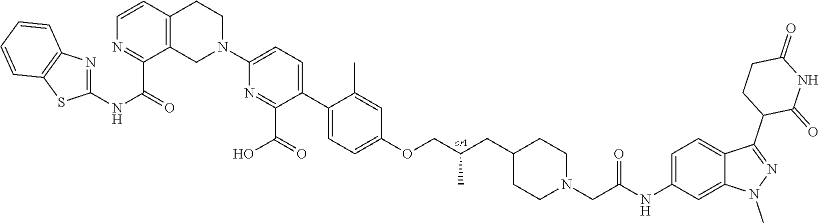 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 141 | 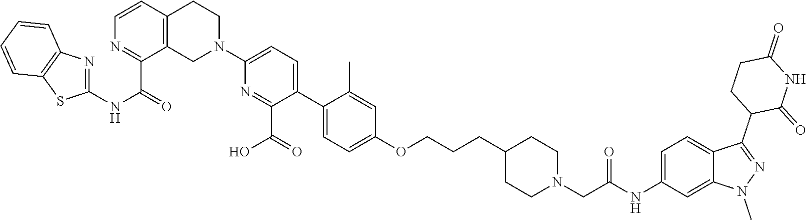 [SMILES: Cc1cc(OCCCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 142 | 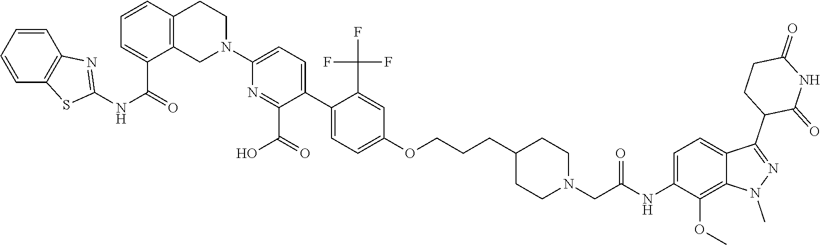 [SMILES: COc1c(NC(=O)CN2CCC(CCCOc3ccc(-c4ccc(N5CCc6cccc(C(=O)Nc7nc8ccccc8s7)c6C5)nc4C(=O)O)c(C(F)(F)F)c3)CC2)ccc2c(C3CCC(=O)NC3=O)nn(C)c12] |
| 143 | 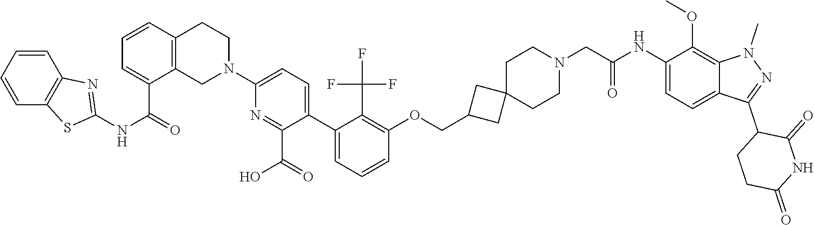 [SMILES: COc1c(NC(=O)CN2CCC3(CC2)CC(COc2cccc(-c4ccc(N5CCc6cccc(C(=O)Nc7nc8ccccc8s7)c6C5)nc4C(=O)O)c2C(F)(F)F)C3)ccc2c(C3CCC(=O)NC3=O)nn(C)c12] |
| 144 | 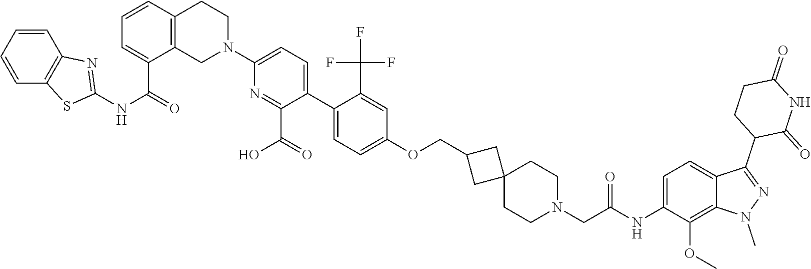 [SMILES: COc1c(NC(=O)CN2CCC3(CC2)CC(COc2ccc(-c4ccc(N5CCc6cccc(C(=O)Nc7nc8ccccc8s7)c6C5)nc4C(=O)O)c(C(F)(F)F)c2)C3)ccc2c(C3CCC(=O)NC3=O)nn(C)c12] |
| 145 | 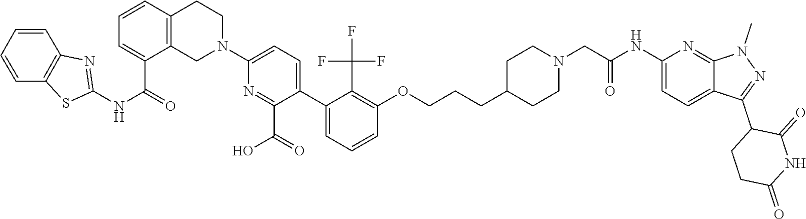 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)nc21] |
| 146 | 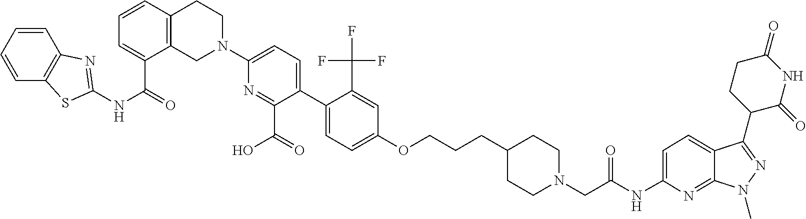 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(C(F)(F)F)c4)CC3)nc21] |
| 147 | 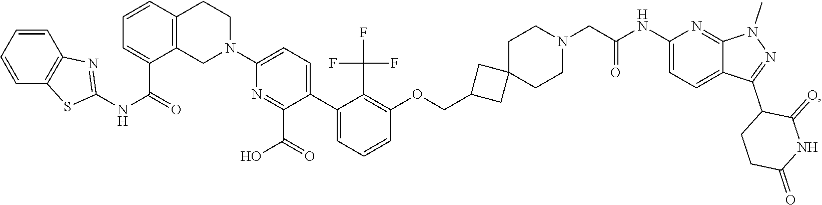 [SMILES: Cn1nc(C2CCC(=O)NC2=O)c2ccc(NC(=O)CN3CCC4(CC3)CC(COc3cccc(-c5ccc(N6CCc7cccc(C(=O)Nc8nc9ccccc9s8)c7C6)nc5C(=O)O)c3C(F)(F)F)C4)nc21] |
| 148 | 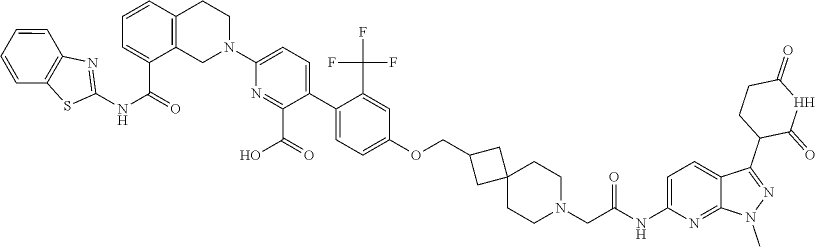 |
| 149 | 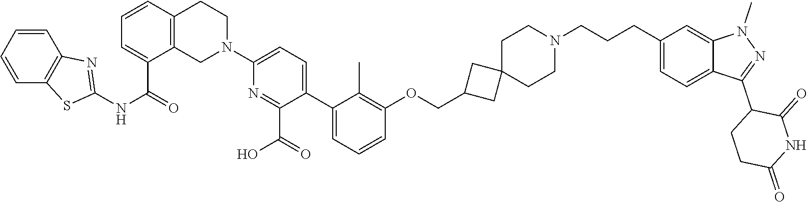 [SMILES: Cc1c(OCC2CC3(CCN(CCCc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 150 | 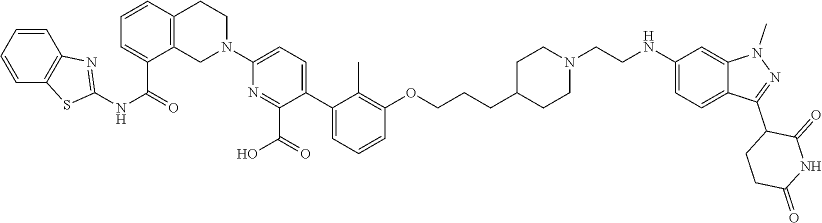 [SMILES: Cc1c(OCCCC2CCN(CCNc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 151 | 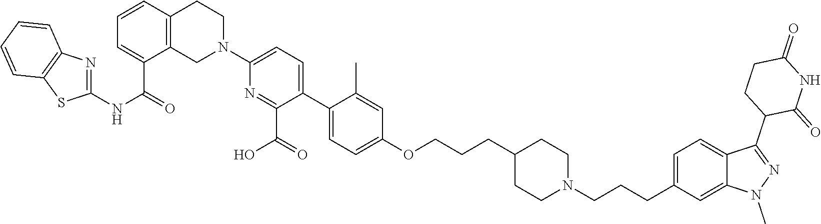 [SMILES: Cc1cc(OCCCC2CCN(CCCc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 152 | 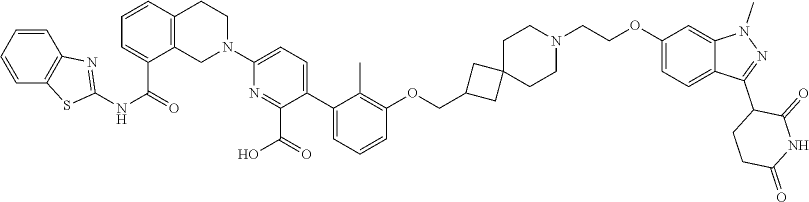 [SMILES: Cc1c(OCC2CC3(CCN(CCOc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 153 | 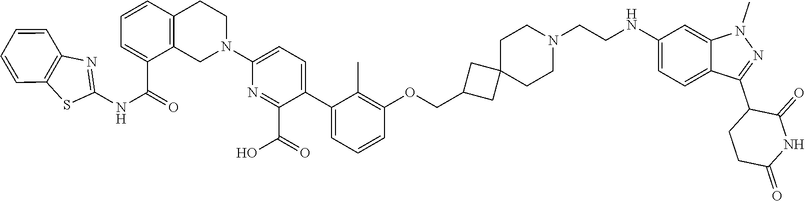 [SMILES: Cc1c(OCC2CC3(CCN(CCNc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 154 | 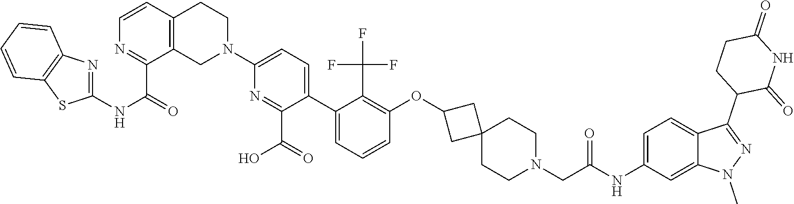 [SMILES: Cn1nc(C2CCC(=O)NC2=O)c2ccc(NC(=O)CN3CCC4(CC3)CC(Oc3cccc(-c5ccc(N6CCc7ccnc(C(=O)Nc8nc9ccccc9s8)c7C6)nc5C(=O)O)c3C(F)(F)F)C4)cc21] |
| 155 | 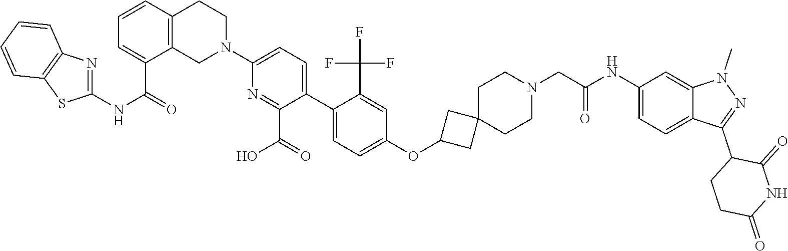 [SMILES: Cn1nc(C2CCC(=O)NC2=O)c2ccc(NC(=O)CN3CCC4(CC3)CC(Oc3ccc(-c5ccc(N6CCc7cccc(C(=O)Nc8nc9ccccc9s8)c7C6)nc5C(=O)O)c(C(F)(F)F)c3)C4)cc21] |
| 156 | 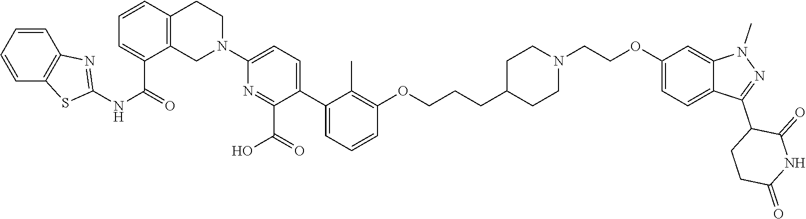 [SMILES: Cc1c(OCCCC2CCN(CCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 157 | 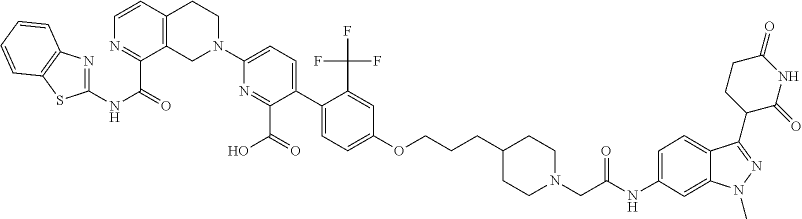 [SMILES: Cn1nc(C2CCC(=O)NC2=O)c2ccc(NC(=O)CN3CCC(CCCOc4ccc(-c5ccc(N6CCc7ccnc(C(=O)Nc8nc9ccccc9s8)c7C6)nc5C(=O)O)c(C(F)(F)F)c4)CC3)cc21] |
| 158 | 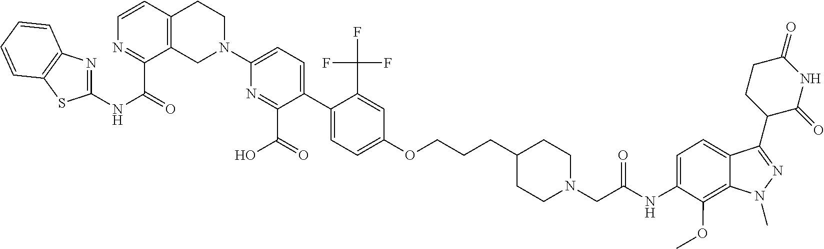 [SMILES: COc1c(NC(=O)CN2CCC(CCCOc3ccc(-c4ccc(N5CCc6ccnc(C(=O)Nc7nc8ccccc8s7)c6C5)nc4C(=O)O)c(C(F)(F)F)c3)CC2)ccc2c(C3CCC(=O)NC3=O)nn(C)c12] |
| 159 | 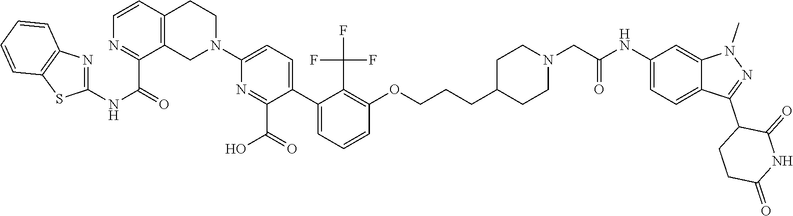 [SMILES: Cn1nc(C2CCC(=O)NC2=O)c2ccc(NC(=O)CN3CCC(CCCOc4cccc(-c5ccc(N6CCc7ccnc(C(=O)Nc8nc9ccccc9s8)c7C6)nc5C(=O)O)c4C(F)(F)F)CC3)cc21] |
| 160 | 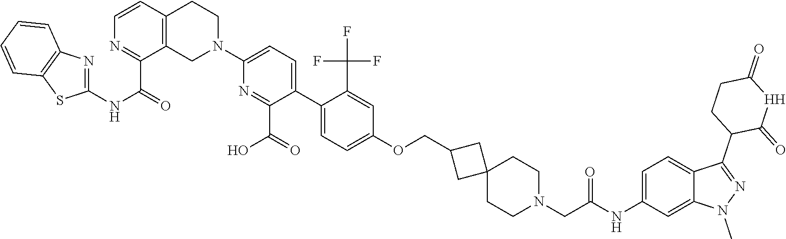 |
| 161 | 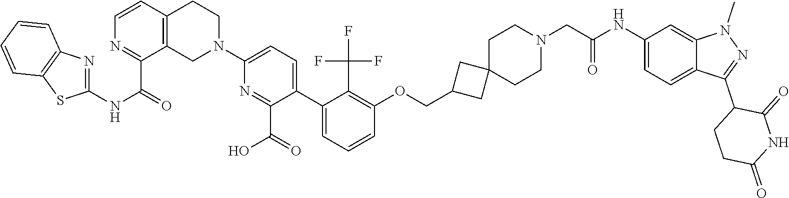 [SMILES: Cn1nc(C2CCC(=O)NC2=O)c2ccc(NC(=O)CN3CCC4(CC3)CC(COc3cccc(-c5ccc(N6CCc7ccnc(C(=O)Nc8nc9ccccc9s8)c7C6)nc5C(=O)O)c3C(F)(F)F)C4)cc21] |
| 162 | 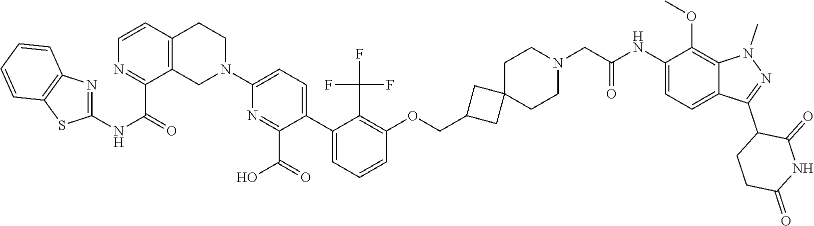 [SMILES: COc1c(NC(=O)CN2CCC3(CC2)CC(COc2cccc(-c4ccc(N5CCc6ccnc(C(=O)Nc7nc8ccccc8s7)c6C5)nc4C(=O)O)c2C(F)(F)F)C3)ccc2c(C3CCC(=O)NC3=O)nn(C)c12] |
| 163 | 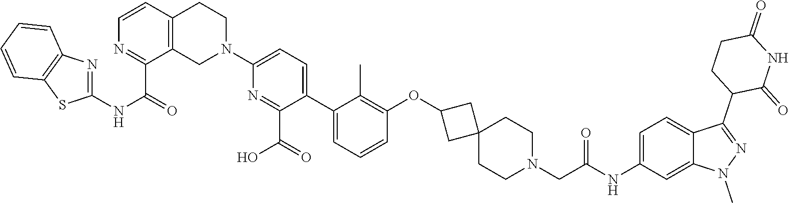 [SMILES: Cc1c(OC2CC3(CCN(CC(=O)Nc4ccc5c(C6CCC(=O)NC6=O)nn(C)c5c4)CC3)C2)cccc1-c1ccc(N2CCc3ccnc(C(=O)Nc4nc5ccccc5s4)c3C2)nc1C(=O)O] |
| 164 | 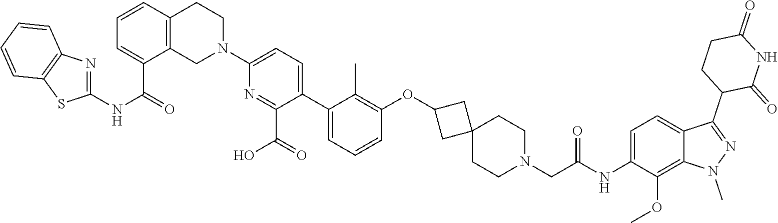 [SMILES: COc1c(NC(=O)CN2CCC3(CC2)CC(Oc2cccc(-c4ccc(N5CCc6cccc(C(=O)Nc7nc8ccccc8s7)c6C5)nc4C(=O)O)c2C)C3)ccc2c(C3CCC(=O)NC3=O)nn(C)c12] |
| 165 | 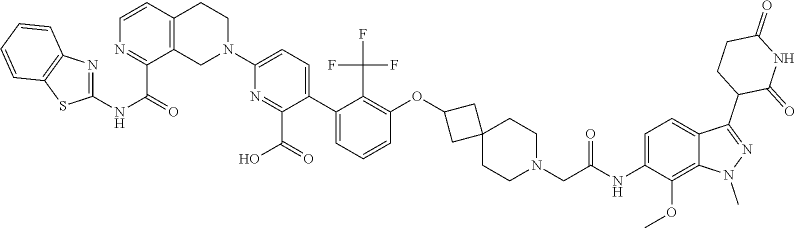 [SMILES: COc1c(NC(=O)CN2CCC3(CC2)CC(Oc2cccc(-c4ccc(N5CCc6ccnc(C(=O)Nc7nc8ccccc8s7)c6C5)nc4C(=O)O)c2C(F)(F)F)C3)ccc2c(C3CCC(=O)NC3=O)nn(C)c12] |
| 166 | 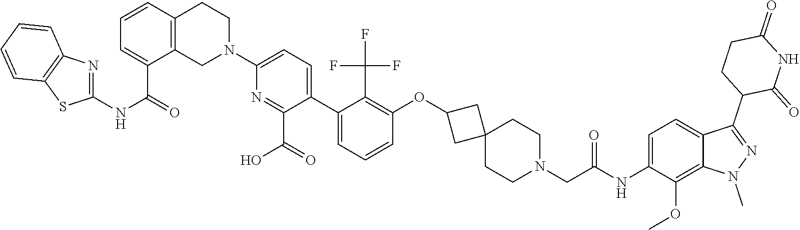 [SMILES: COc1c(NC(=O)CN2CCC3(CC2)CC(Oc2cccc(-c4ccc(N5CCc6cccc(C(=O)Nc7nc8ccccc8s7)c6C5)nc4C(=O)O)c2C(F)(F)F)C3)ccc2c(C3CCC(=O)NC3=O)nn(C)c12] |
| 167 | 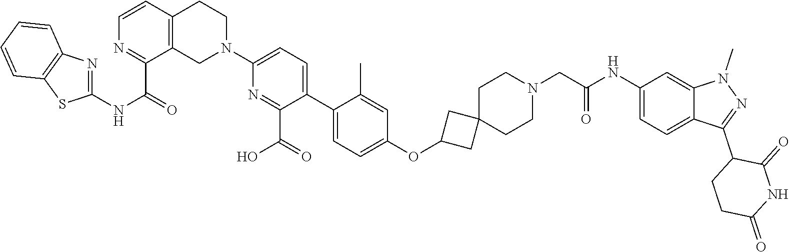 [SMILES: Cc1cc(OC2CC3(CCN(CC(=O)Nc4ccc5c(C6CCC(=O)NC6=O)nn(C)c5c4)CC3)C2)ccc1-c1ccc(N2CCc3ccnc(C(=O)Nc4nc5ccccc5s4)c3C2)nc1C(=O)O] |
| 168 | 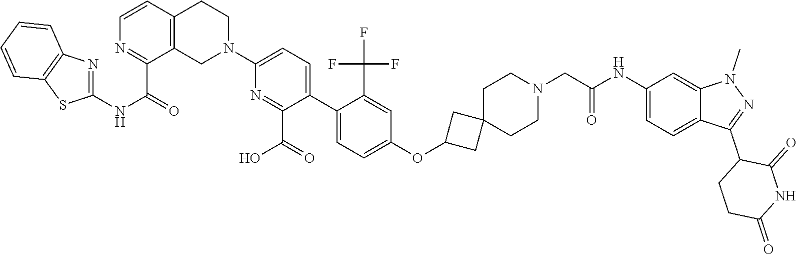 [SMILES: Cn1nc(C2CCC(=O)NC2=O)c2ccc(NC(=O)CN3CCC4(CC3)CC(Oc3ccc(-c5ccc(N6CCc7ccnc(C(=O)Nc8nc9ccccc9s8)c7C6)nc5C(=O)O)c(C(F)(F)F)c3)C4)cc21] |
| 169 | 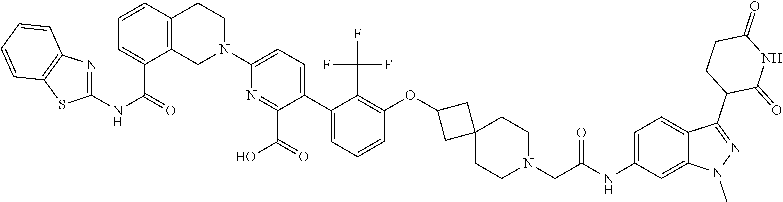 [SMILES: Cn1nc(C2CCC(=O)NC2=O)c2ccc(NC(=O)CN3CCC4(CC3)CC(Oc3cccc(-c5ccc(N6CCc7cccc(C(=O)Nc8nc9ccccc9s8)c7C6)nc5C(=O)O)c3C(F)(F)F)C4)cc21] |
| 170 | 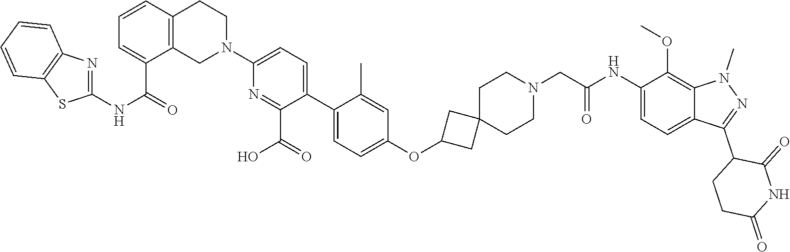 [SMILES: COc1c(NC(=O)CN2CCC3(CC2)CC(Oc2ccc(-c4ccc(N5CCc6cccc(C(=O)Nc7nc8ccccc8s7)c6C5)nc4C(=O)O)c(C)c2)C3)ccc2c(C3CCC(=O)NC3=O)nn(C)c12] |
| 171 | 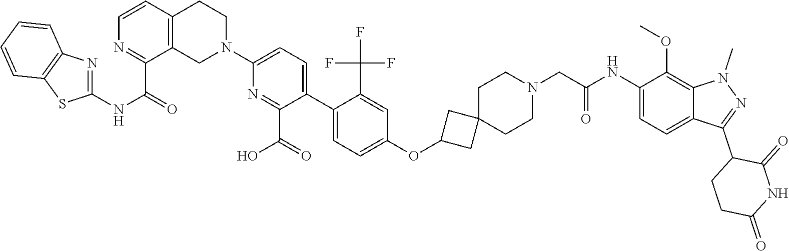 [SMILES: COc1c(NC(=O)CN2CCC3(CC2)CC(Oc2ccc(-c4ccc(N5CCc6ccnc(C(=O)Nc7nc8ccccc8s7)c6C5)nc4C(=O)O)c(C(F)(F)F)c2)C3)ccc2c(C3CCC(=O)NC3=O)nn(C)c12] |
| 172 | 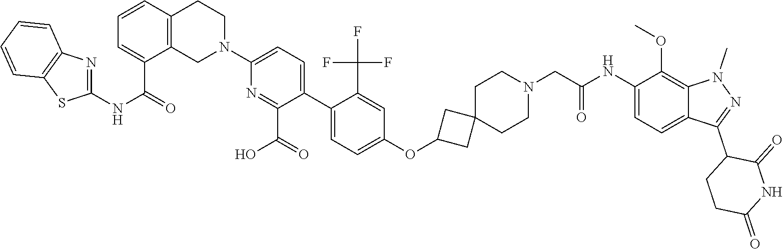 [SMILES: COc1c(NC(=O)CN2CCC3(CC2)CC(Oc2ccc(-c4ccc(N5CCc6cccc(C(=O)Nc7nc8ccccc8s7)c6C5)nc4C(=O)O)c(C(F)(F)F)c2)C3)ccc2c(C3CCC(=O)NC3=O)nn(C)c12] |
| 173 | 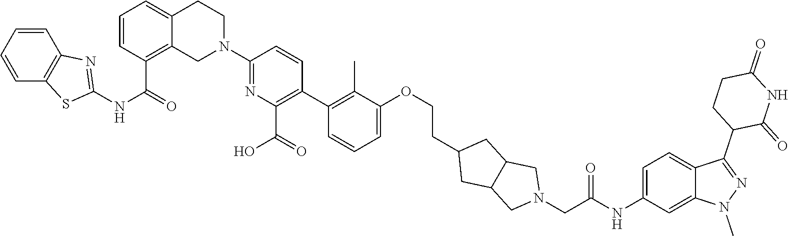 [SMILES: Cc1c(OCCC2CC3CN(CC(=O)Nc4ccc5c(C6CCC(=O)NC6=O)nn(C)c5c4)CC3C2)cccc1-c1ccc(N2CCc3cccc(C(=O)Nc4nc5ccccc5s4)c3C2)nc1C(=O)O] |
| 173a | 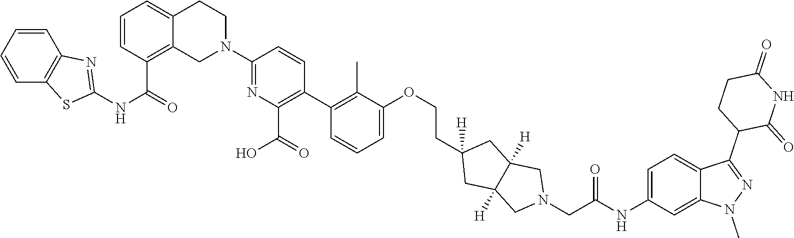 [SMILES: Cc1c(OCC[C@@H]2C[C@@H]3CN(CC(=O)Nc4ccc5c(C6CCC(=O)NC6=O)nn(C)c5c4)C[C@@H]3C2)cccc1-c1ccc(N2CCc3cccc(C(=O)Nc4nc5ccccc5s4)c3C2)nc1C(=O)O] |
| 174 | 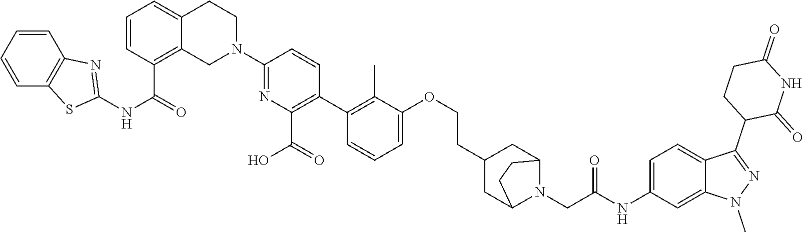 [SMILES: Cc1c(OCCC2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 174a | 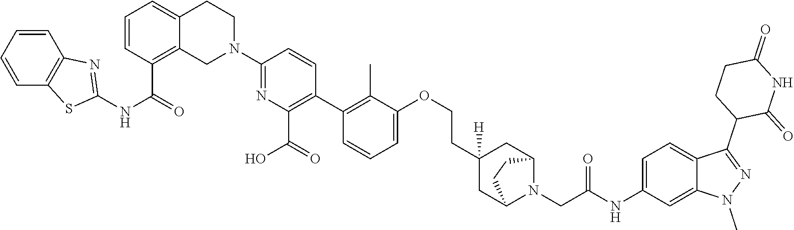 [SMILES: Cc1c(OCC[C@@H]2C[C@H]3CC[C@@H](C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 175 | 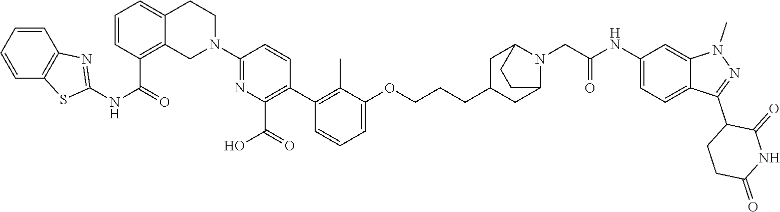 [SMILES: Cc1c(OCCCC2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 175a | 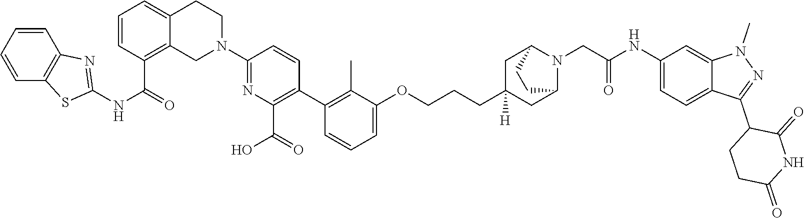 [SMILES: Cc1c(OCCC[C@@H]2C[C@H]3CC[C@@H](C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 176 | 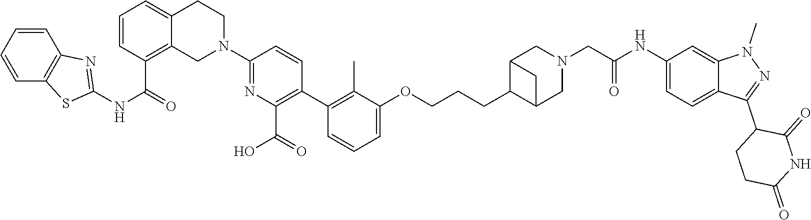 [SMILES: Cc1c(OCCCC2C3CC2CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O] |
| 176a | 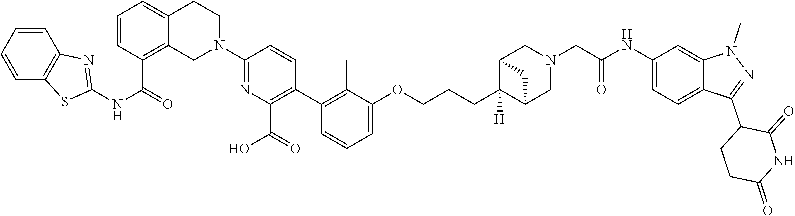 [SMILES: Cc1c(OCCC[C@H]2[C@@H]3C[C@H]2CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O] |
| 177 | 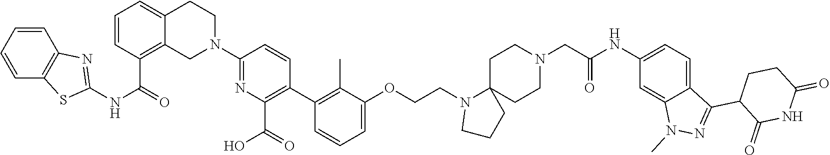 [SMILES: Cc1c(OCCN2CCCC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 178 | 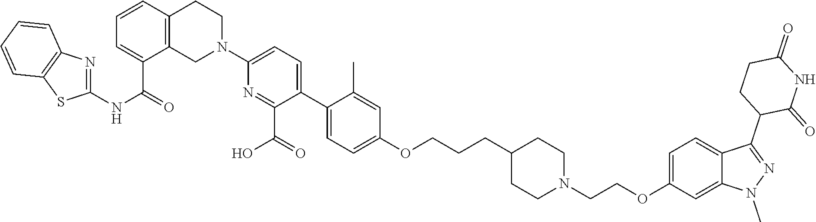 [SMILES: Cc1cc(OCCCC2CCN(CCOc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 179 | 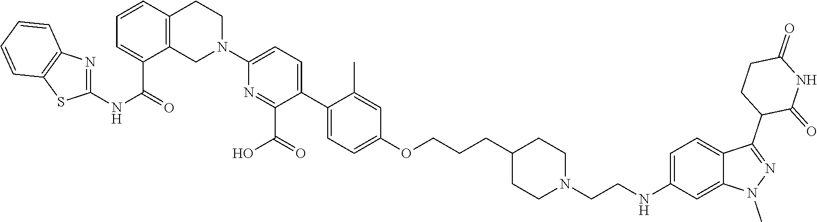 [SMILES: Cc1cc(OCCCC2CCN(CCNc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 180 | 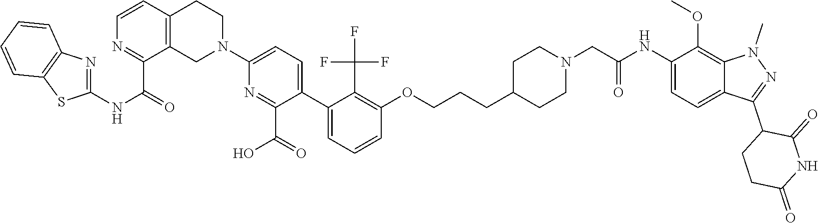 [SMILES: COc1c(NC(=O)CN2CCC(CCCOc3cccc(-c4ccc(N5CCc6ccnc(C(=O)Nc7nc8ccccc8s7)c6C5)nc4C(=O)O)c3C(F)(F)F)CC2)ccc2c(C3CCC(=O)NC3=O)nn(C)c12] |
| 181 | 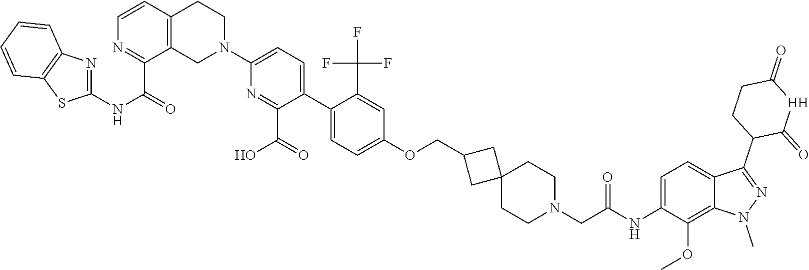 |
| 182 | 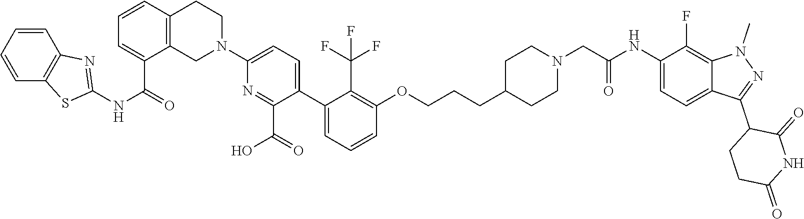 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)c(F)c21] |
| 183 | 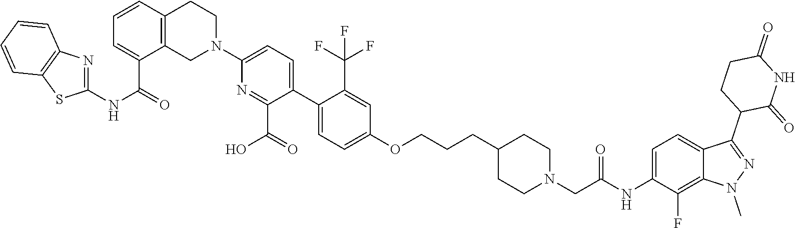 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(C(F)(F)F)c4)CC3)c(F)c21] |
| 184 | 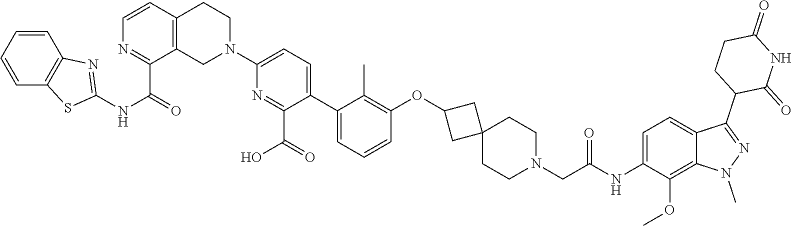 [SMILES: COc1c(NC(=O)CN2CCC3(CC2)CC(Oc2cccc(-c4ccc(N5CCc6ccnc(C(=O)Nc7nc8ccccc8s7)c6C5)nc4C(=O)O)c2C)C3)ccc2c(C3CCC(=O)NC3=O)nn(C)c12] |
| 185 | 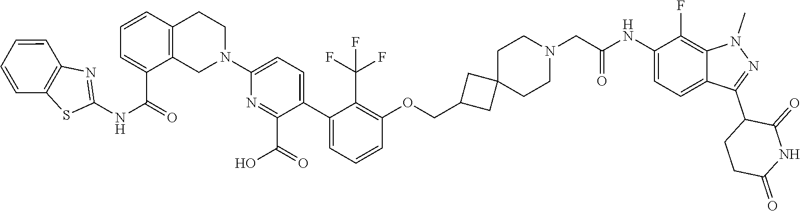 [SMILES: Cn1nc(C2CCC(=O)NC2=O)c2ccc(NC(=O)CN3CCC4(CC3)CC(COc3cccc(-c5ccc(N6CCc7cccc(C(=O)Nc8nc9ccccc9s8)c7C6)nc5C(=O)O)c3C(F)(F)F)C4)c(F)c21] |
| 186 | 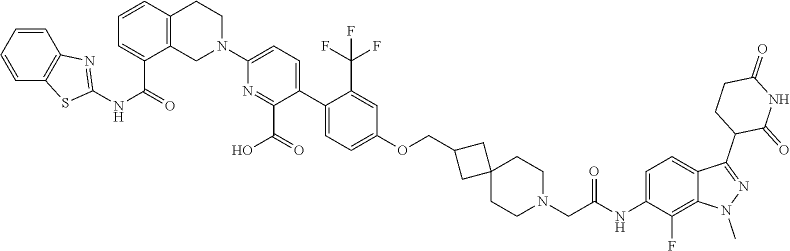 [SMILES: Cn1nc(C2CCC(=O)NC2=O)c2ccc(NC(=O)CN3CCC4(CC3)CC(COc3ccc(-c5ccc(N6CCc7cccc(C(=O)Nc8nc9ccccc9s8)c7C6)nc5C(=O)O)c(C(F)(F)F)c3)C4)c(F)c21] |
| 187 | 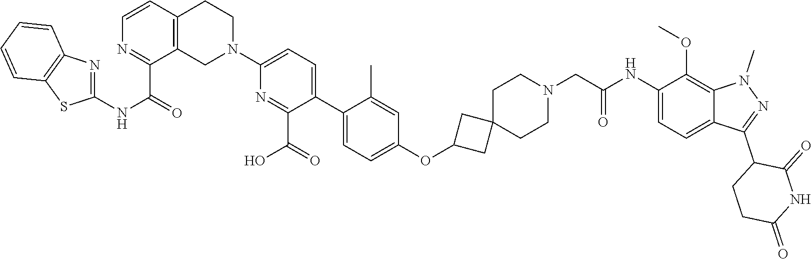 [SMILES: COc1c(NC(=O)CN2CCC3(CC2)CC(Oc2ccc(-c4ccc(N5CCc6ccnc(C(=O)Nc7nc8ccccc8s7)c6C5)nc4C(=O)O)c(C)c2)C3)ccc2c(C3CCC(=O)NC3=O)nn(C)c12] |
| 188 | 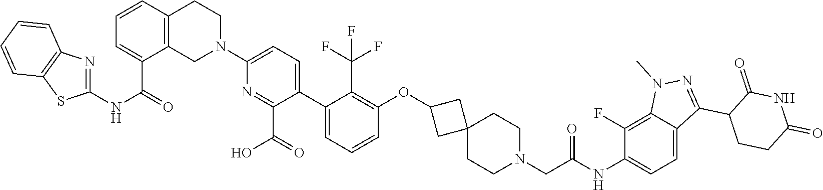 [SMILES: Cn1nc(C2CCC(=O)NC2=O)c2ccc(NC(=O)CN3CCC4(CC3)CC(Oc3cccc(-c5ccc(N6CCc7cccc(C(=O)Nc8nc9ccccc9s8)c7C6)nc5C(=O)O)c3C(F)(F)F)C4)c(F)c21] |
| 189 | 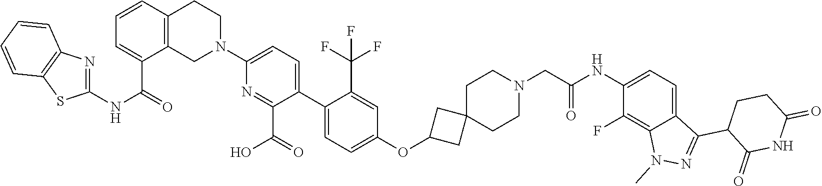 [SMILES: Cn1nc(C2CCC(=O)NC2=O)c2ccc(NC(=O)CN3CCC4(CC3)CC(Oc3ccc(-c5ccc(N6CCc7cccc(C(=O)Nc8nc9ccccc9s8)c7C6)nc5C(=O)O)c(C(F)(F)F)c3)C4)c(F)c21] |
| 190 | 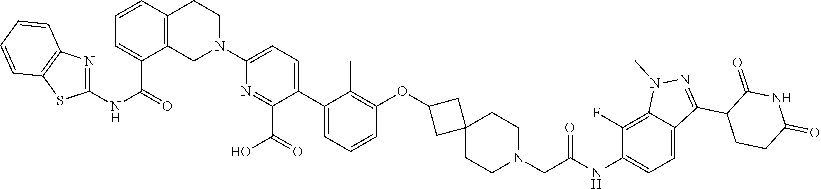 [SMILES: Cc1c(OC2CC3(CCN(CC(=O)Nc4ccc5c(C6CCC(=O)NC6=O)nn(C)c5c4F)CC3)C2)cccc1-c1ccc(N2CCc3cccc(C(=O)Nc4nc5ccccc5s4)c3C2)nc1C(=O)O] |
| 191 | 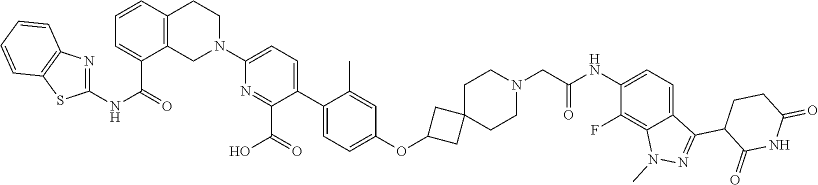 [SMILES: Cc1cc(OC2CC3(CCN(CC(=O)Nc4ccc5c(C6CCC(=O)NC6=O)nn(C)c5c4F)CC3)C2)ccc1-c1ccc(N2CCc3cccc(C(=O)Nc4nc5ccccc5s4)c3C2)nc1C(=O)O] |
| 192 | 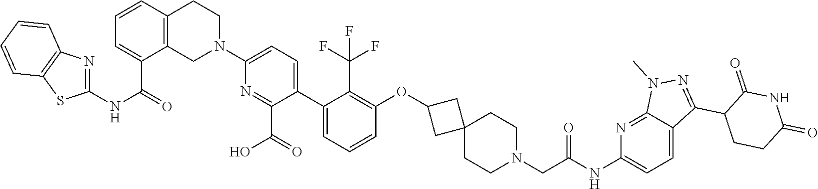 [SMILES: Cn1nc(C2CCC(=O)NC2=O)c2ccc(NC(=O)CN3CCC4(CC3)CC(Oc3cccc(-c5ccc(N6CCc7cccc(C(=O)Nc8nc9ccccc9s8)c7C6)nc5C(=O)O)c3C(F)(F)F)C4)nc21] |
| 193 | 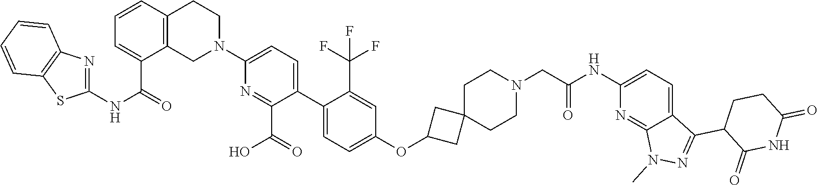 [SMILES: Cn1nc(C2CCC(=O)NC2=O)c2ccc(NC(=O)CN3CCC4(CC3)CC(Oc3ccc(-c5ccc(N6CCc7cccc(C(=O)Nc8nc9ccccc9s8)c7C6)nc5C(=O)O)c(C(F)(F)F)c3)C4)nc21] |
| 194 | 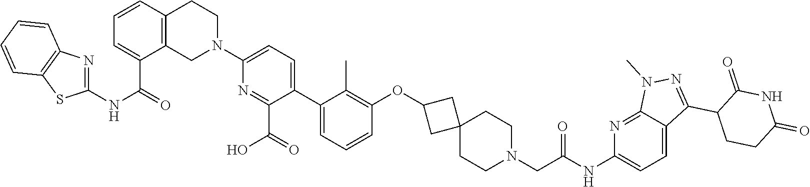 [SMILES: Cc1c(OC2CC3(CCN(CC(=O)Nc4ccc5c(C6CCC(=O)NC6=O)nn(C)c5n4)CC3)C2)cccc1-c1ccc(N2CCc3cccc(C(=O)Nc4nc5ccccc5s4)c3C2)nc1C(=O)O] |
| 195 | 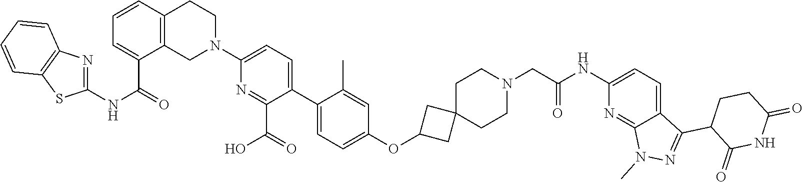 [SMILES: Cc1cc(OC2CC3(CCN(CC(=O)Nc4ccc5c(C6CCC(=O)NC6=O)nn(C)c5n4)CC3)C2)ccc1-c1ccc(N2CCc3cccc(C(=O)Nc4nc5ccccc5s4)c3C2)nc1C(=O)O] |
| 196 | 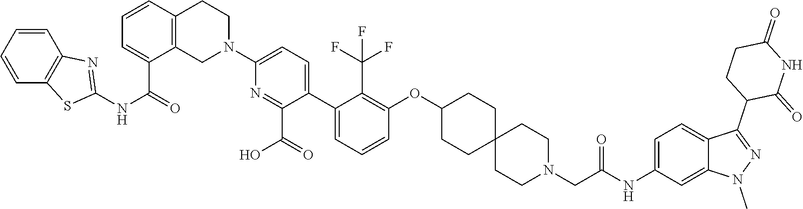 [SMILES: Cn1nc(C2CCC(=O)NC2=O)c2ccc(NC(=O)CN3CCC4(CCC(Oc5cccc(-c6ccc(N7CCc8cccc(C(=O)Nc9nc%10ccccc%10s9)c8C7)nc6C(=O)O)c5C(F)(F)F)CC4)CC3)cc21] |
| 197 | 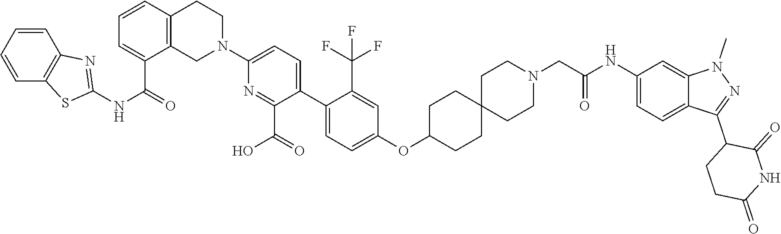 [SMILES: Cn1nc(C2CCC(=O)NC2=O)c2ccc(NC(=O)CN3CCC4(CCC(Oc5ccc(-c6ccc(N7CCc8cccc(C(=O)Nc9nc%10ccccc%10s9)c8C7)nc6C(=O)O)c(C(F)(F)F)c5)CC4)CC3)cc21] |
| 198 | 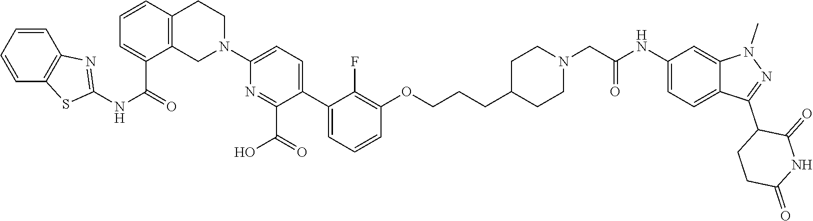 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4F)CC3)cc21] |
| 199 | 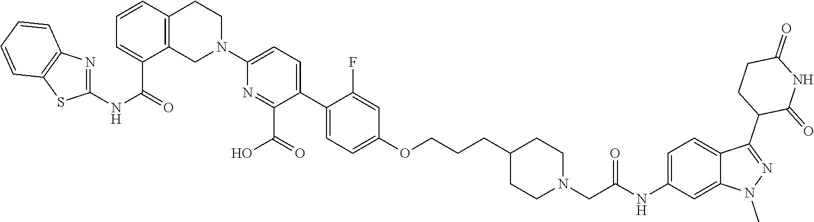 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(F)c4)CC3)cc21] |
| 200 | 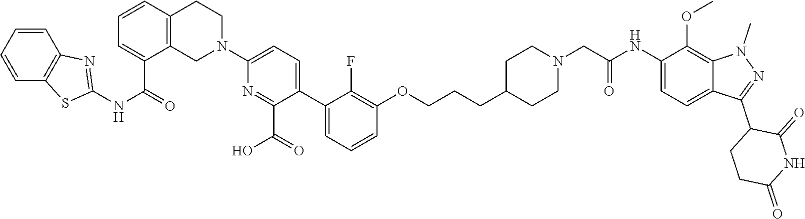 [SMILES: COc1c(NC(=O)CN2CCC(CCCOc3cccc(-c4ccc(N5CCc6cccc(C(=O)Nc7nc8ccccc8s7)c6C5)nc4C(=O)O)c3F)CC2)ccc2c(C3CCC(=O)NC3=O)nn(C)c12] |
| 201 | 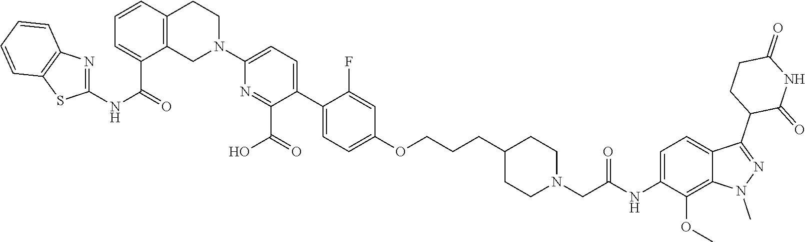 [SMILES: COc1c(NC(=O)CN2CCC(CCCOc3ccc(-c4ccc(N5CCc6cccc(C(=O)Nc7nc8ccccc8s7)c6C5)nc4C(=O)O)c(F)c3)CC2)ccc2c(C3CCC(=O)NC3=O)nn(C)c12] |
| 202 | 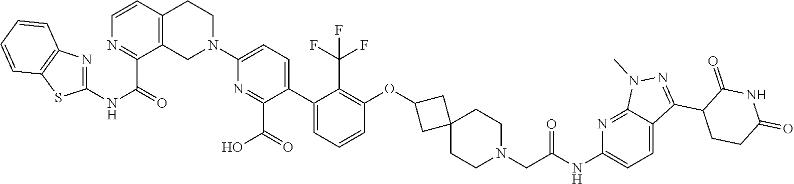 [SMILES: Cn1nc(C2CCC(=O)NC2=O)c2ccc(NC(=O)CN3CCC4(CC3)CC(Oc3cccc(-c5ccc(N6CCc7ccnc(C(=O)Nc8nc9ccccc9s8)c7C6)nc5C(=O)O)c3C(F)(F)F)C4)nc21] |
| 203 | 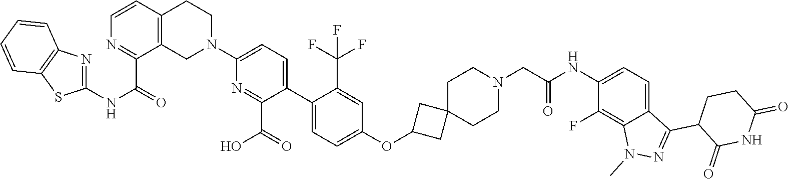 [SMILES: Cn1nc(C2CCC(=O)NC2=O)c2ccc(NC(=O)CN3CCC4(CC3)CC(Oc3ccc(-c5ccc(N6CCc7ccnc(C(=O)Nc8nc9ccccc9s8)c7C6)nc5C(=O)O)c(C(F)(F)F)c3)C4)c(F)c21] |
| 204 | 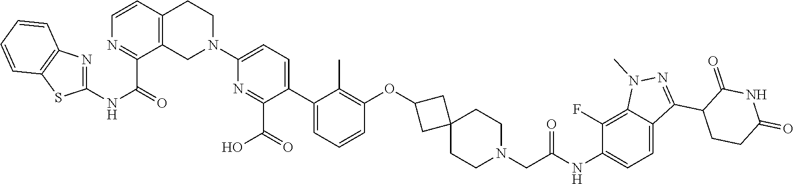 [SMILES: Cc1c(OC2CC3(CCN(CC(=O)Nc4ccc5c(C6CCC(=O)NC6=O)nn(C)c5c4F)CC3)C2)cccc1-c1ccc(N2CCc3ccnc(C(=O)Nc4nc5ccccc5s4)c3C2)nc1C(=O)O] |
| 205 | 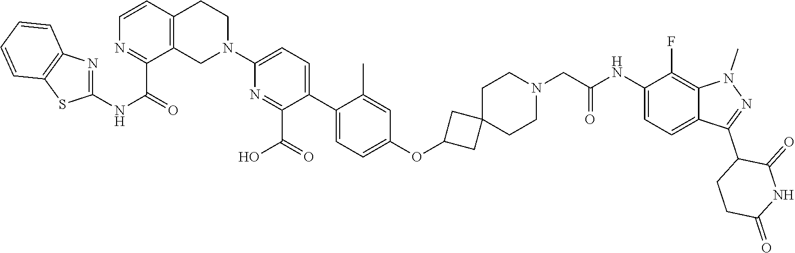 [SMILES: Cc1cc(OC2CC3(CCN(CC(=O)Nc4ccc5c(C6CCC(=O)NC6=O)nn(C)c5c4F)CC3)C2)ccc1-c1ccc(N2CCc3ccnc(C(=O)Nc4nc5ccccc5s4)c3C2)nc1C(=O)O] |
| 206 | 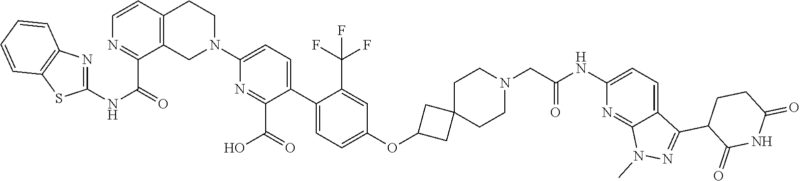 [SMILES: Cn1nc(C2CCC(=O)NC2=O)c2ccc(NC(=O)CN3CCC4(CC3)CC(Oc3ccc(-c5ccc(N6CCc7ccnc(C(=O)Nc8nc9ccccc9s8)c7C6)nc5C(=O)O)c(C(F)(F)F)c3)C4)nc21] |
| 207 | 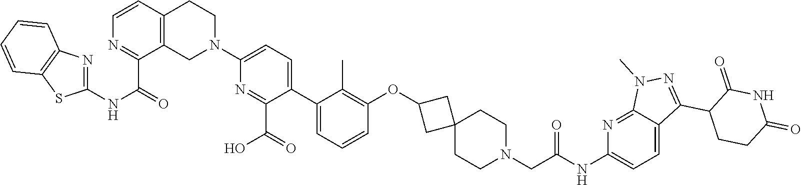 [SMILES: Cc1c(OC2CC3(CCN(CC(=O)Nc4ccc5c(C6CCC(=O)NC6=O)nn(C)c5n4)CC3)C2)cccc1-c1ccc(N2CCc3ccnc(C(=O)Nc4nc5ccccc5s4)c3C2)nc1C(=O)O] |
| 208 | 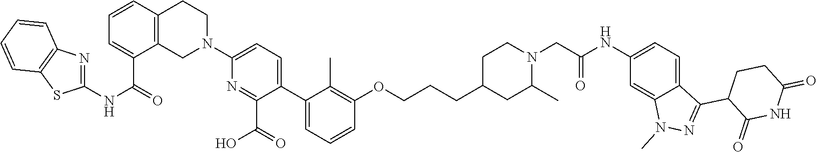 [SMILES: Cc1c(OCCCC2CCN(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| 208a | 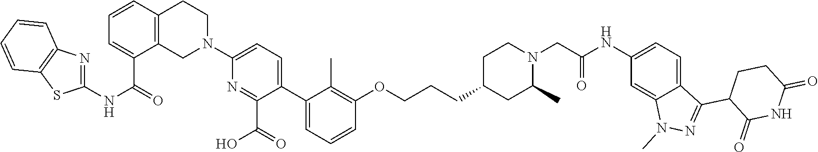 [SMILES: Cc1c(OCCC[C@@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 208b | 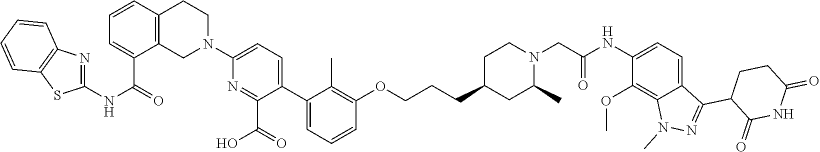 [SMILES: COc1c(NC(=O)CN2CC[C@H](CCCOc3cccc(-c4ccc(N5CCc6cccc(C(=O)Nc7nc8ccccc8s7)c6C5)nc4C(=O)O)c3C)C[C@@H]2C)ccc2c(C3CCC(=O)NC3=O)nn(C)c12] |
| 208c | 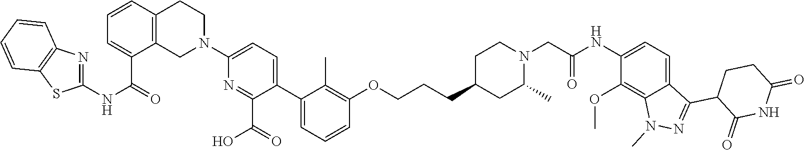 [SMILES: COc1c(NC(=O)CN2CC[C@H](CCCOc3cccc(-c4ccc(N5CCc6cccc(C(=O)Nc7nc8ccccc8s7)c6C5)nc4C(=O)O)c3C)C[C@H]2C)ccc2c(C3CCC(=O)NC3=O)nn(C)c12] |
| 208d | 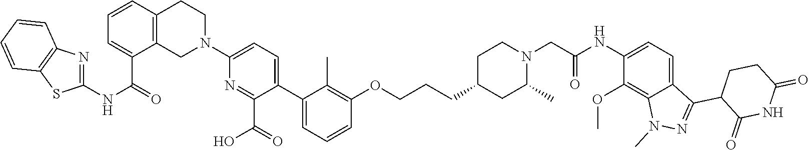 [SMILES: COc1c(NC(=O)CN2CC[C@@H](CCCOc3cccc(-c4ccc(N5CCc6cccc(C(=O)Nc7nc8ccccc8s7)c6C5)nc4C(=O)O)c3C)C[C@H]2C)ccc2c(C3CCC(=O)NC3=O)nn(C)c12] |
| 209 | 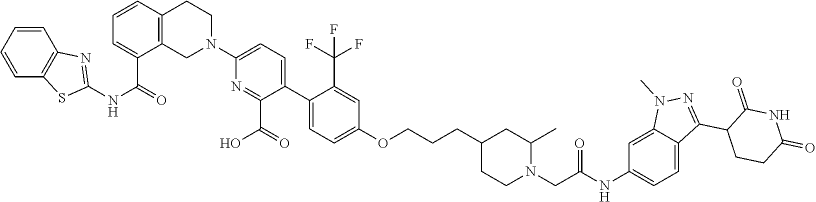 [SMILES: CC1CC(CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1] |
| 209a | 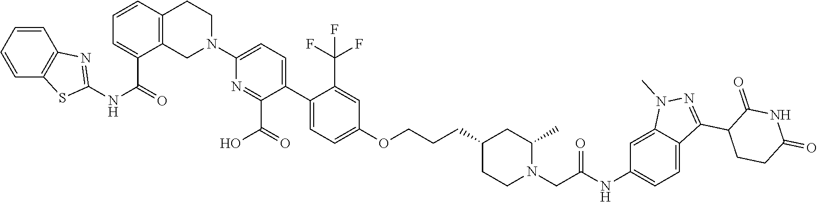 [SMILES: C[C@H]1C[C@@H](CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1] |
| 209b | 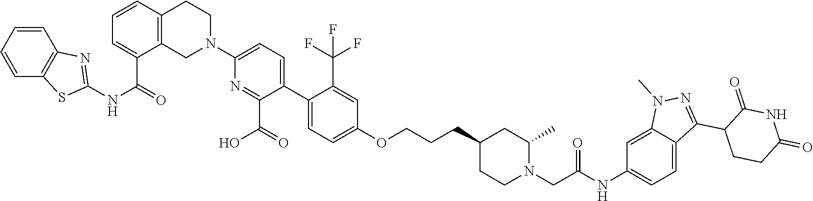 [SMILES: C[C@H]1C[C@H](CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1] |
| 209c | 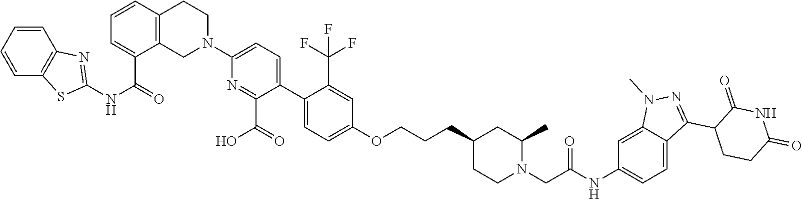 [SMILES: C[C@@H]1C[C@H](CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1] |
| 209d | 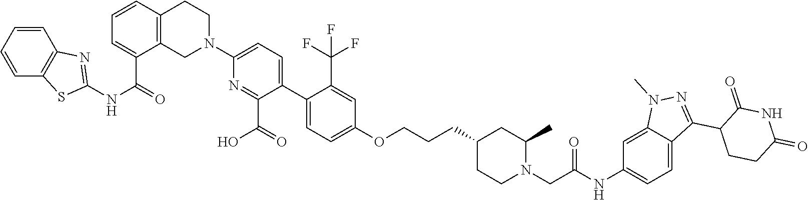 [SMILES: C[C@@H]1C[C@@H](CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1] |
| 210 | 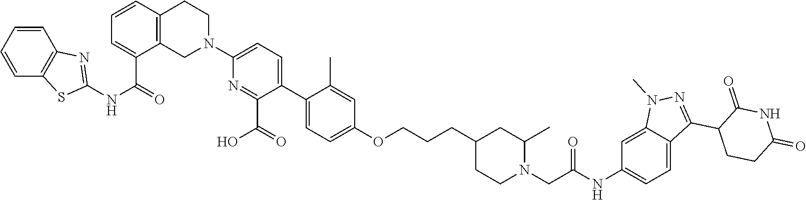 [SMILES: Cc1cc(OCCCC2CCN(CC(=O)Nc3ccc4c(C5CCC(=O)NC5=O)nn(C)c4c3)C(C)C2)ccc1-c1ccc(N2CCc3cccc(C(=O)Nc4nc5ccccc5s4)c3C2)nc1C(=O)O] |
| 210a | 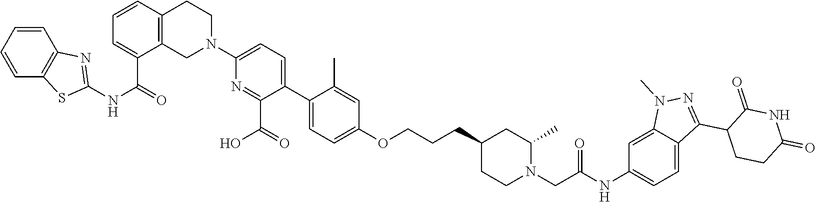 [SMILES: Cc1cc(OCCC[C@@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O] |
| 210b | 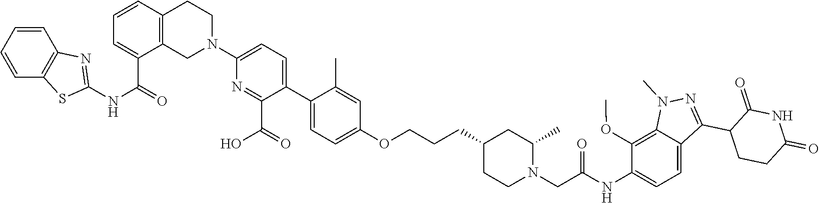 [SMILES: COc1c(NC(=O)CN2CC[C@H](CCCOc3ccc(-c4ccc(N5CCc6cccc(C(=O)Nc7nc8ccccc8s7)c6C5)nc4C(=O)O)c(C)c3)C[C@@H]2C)ccc2c(C3CCC(=O)NC3=O)nn(C)c12] |
| 210c | 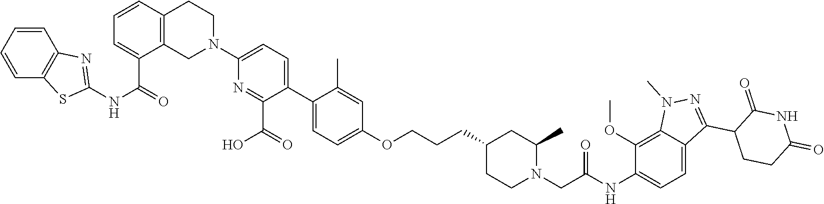 [SMILES: COc1c(NC(=O)CN2CC[C@H](CCCOc3ccc(-c4ccc(N5CCc6cccc(C(=O)Nc7nc8ccccc8s7)c6C5)nc4C(=O)O)c(C)c3)C[C@H]2C)ccc2c(C3CCC(=O)NC3=O)nn(C)c12] |
| 210d | 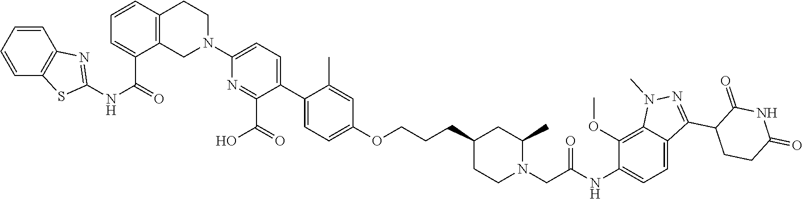 [SMILES: COc1c(NC(=O)CN2CC[C@@H](CCCOc3ccc(-c4ccc(N5CCc6cccc(C(=O)Nc7nc8ccccc8s7)c6C5)nc4C(=O)O)c(C)c3)C[C@H]2C)ccc2c(C3CCC(=O)NC3=O)nn(C)c12] |
| 211 | 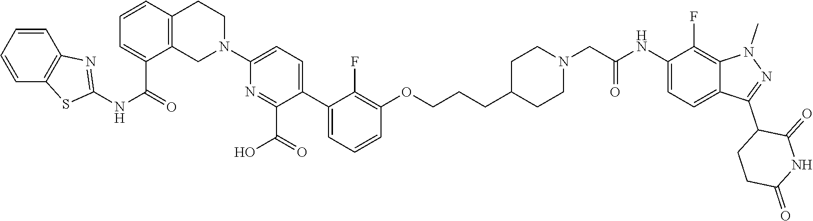 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4F)CC3)c(F)c21] |
| 212 | 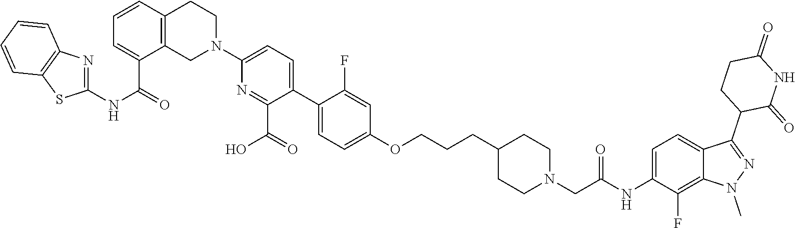 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(F)c4)CC3)c(F)c21] |
| 213 | 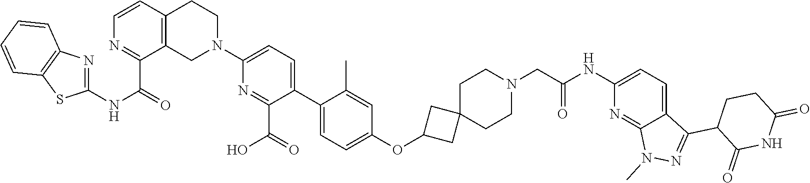 [SMILES: Cc1cc(OC2CC3(CCN(CC(=O)Nc4ccc5c(C6CCC(=O)NC6=O)nn(C)c5n4)CC3)C2)ccc1-c1ccc(N2CCc3ccnc(C(=O)Nc4nc5ccccc5s4)c3C2)nc1C(=O)O] |
| 214 | 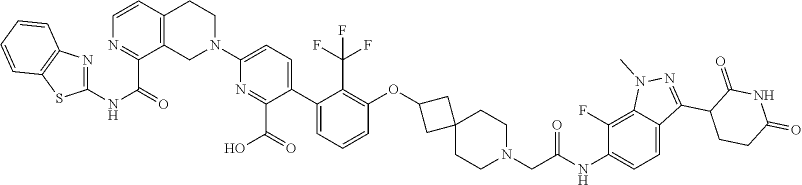 [SMILES: Cn1nc(C2CCC(=O)NC2=O)c2ccc(NC(=O)CN3CCC4(CC3)CC(Oc3cccc(-c5ccc(N6CCc7ccnc(C(=O)Nc8nc9ccccc9s8)c7C6)nc5C(=O)O)c3C(F)(F)F)C4)c(F)c21] |
| 215 | 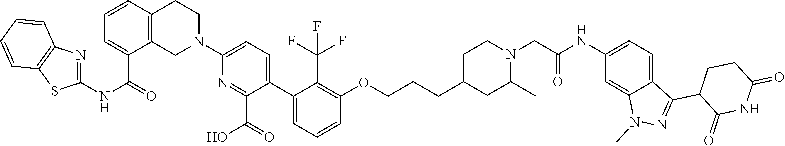 [SMILES: CC1CC(CCCOc2cccc(-c3ccc(N4CCc5cccc(C(=O)Nc6nc7ccccc7s6)c5C4)nc3C(=O)O)c2C(F)(F)F)CCN1CC(=O)Nc1ccc2c(C3CCC(=O)NC3=O)nn(C)c2c1] |
| 215a | 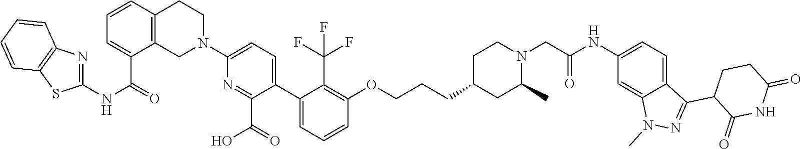 [SMILES: C[C@H]1C[C@H](CCCOc2cccc(-c3ccc(N4CCc5cccc(C(=O)Nc6nc7ccccc7s6)c5C4)nc3C(=O)O)c2C(F)(F)F)CCN1CC(=O)Nc1ccc2c(C3CCC(=O)NC3=O)nn(C)c2c1] |
| 215b | 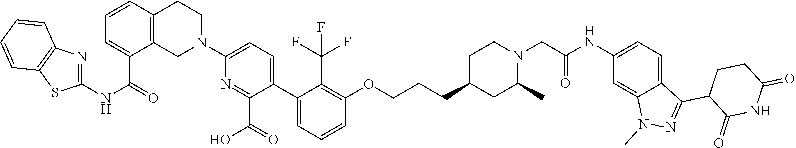 [SMILES: C[C@H]1C[C@@H](CCCOc2cccc(-c3ccc(N4CCc5cccc(C(=O)Nc6nc7ccccc7s6)c5C4)nc3C(=O)O)c2C(F)(F)F)CCN1CC(=O)Nc1ccc2c(C3CCC(=O)NC3=O)nn(C)c2c1] |
| 215c | 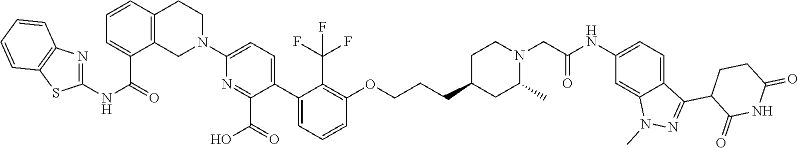 [SMILES: C[C@@H]1C[C@@H](CCCOc2cccc(-c3ccc(N4CCc5cccc(C(=O)Nc6nc7ccccc7s6)c5C4)nc3C(=O)O)c2C(F)(F)F)CCN1CC(=O)Nc1ccc2c(C3CCC(=O)NC3=O)nn(C)c2c1] |
| 215d | 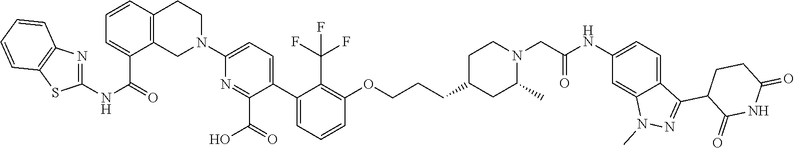 |
| 216 | 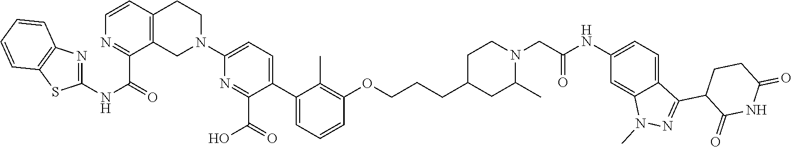 [SMILES: Cc1c(OCCCC2CCN(CC(=O)Nc3ccc4c(C5CCC(=O)NC5=O)nn(C)c4c3)C(C)C2)cccc1-c1ccc(N2CCc3ccnc(C(=O)Nc4nc5ccccc5s4)c3C2)nc1C(=O)O] |
| 216a | 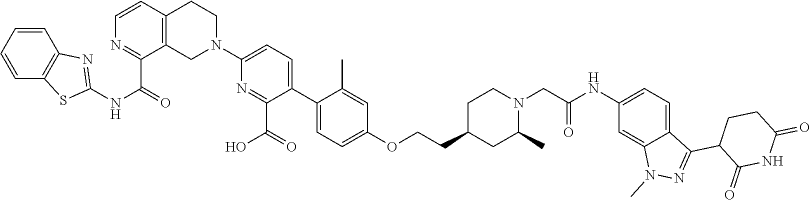 [SMILES: Cc1cc(OCC[C@H]2CCN(CC(=O)Nc3ccc4c(C5CCC(=O)NC5=O)nn(C)c4c3)[C@@H](C)C2)ccc1-c1ccc(N2CCc3ccnc(C(=O)Nc4nc5ccccc5s4)c3C2)nc1C(=O)O] |
| 216b | 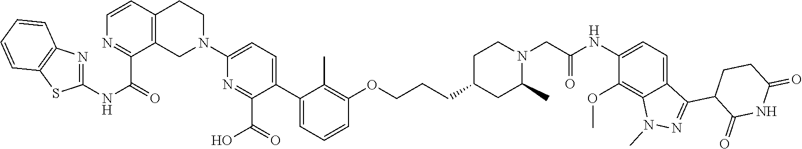 [SMILES: COc1c(NC(=O)CN2CC[C@@H](CCCOc3cccc(-c4ccc(N5CCc6ccnc(C(=O)Nc7nc8ccccc8s7)c6C5)nc4C(=O)O)c3C)C[C@@H]2C)ccc2c(C3CCC(=O)NC3=O)nn(C)c12] |
| 216c | 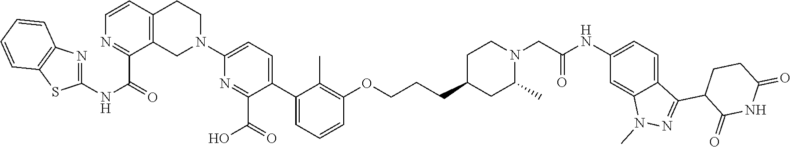 [SMILES: Cc1c(OCCC[C@H]2CCN(CC(=O)Nc3ccc4c(C5CCC(=O)NC5=O)nn(C)c4c3)[C@H](C)C2)cccc1-c1ccc(N2CCc3ccnc(C(=O)Nc4nc5ccccc5s4)c3C2)nc1C(=O)O] |
| 216d | 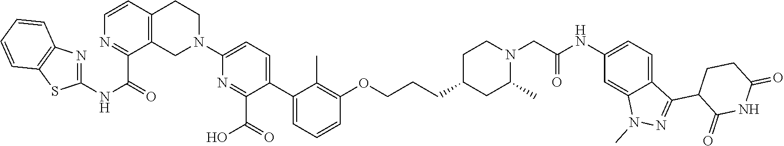 [SMILES: Cc1c(OCCC[C@@H]2CCN(CC(=O)Nc3ccc4c(C5CCC(=O)NC5=O)nn(C)c4c3)[C@H](C)C2)cccc1-c1ccc(N2CCc3ccnc(C(=O)Nc4nc5ccccc5s4)c3C2)nc1C(=O)O] |
| 217 | 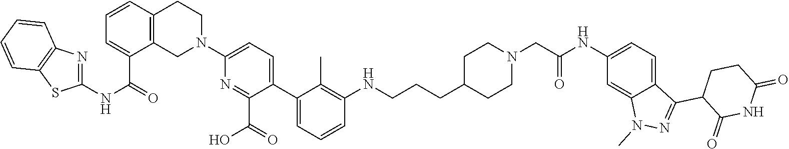 [SMILES: Cc1c(NCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 218 | 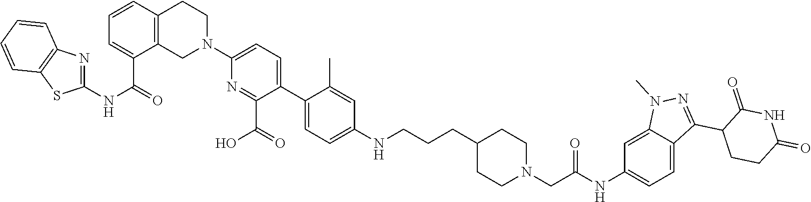 [SMILES: Cc1cc(NCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 219 | 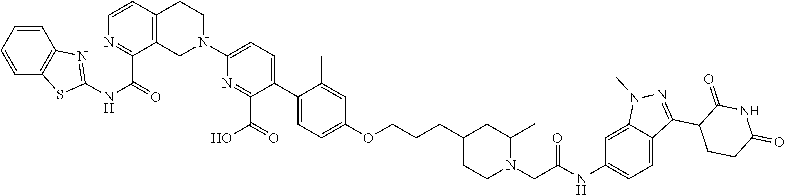 [SMILES: Cc1cc(OCCCC2CCN(CC(=O)Nc3ccc4c(C5CCC(=O)NC5=O)nn(C)c4c3)C(C)C2)ccc1-c1ccc(N2CCc3ccnc(C(=O)Nc4nc5ccccc5s4)c3C2)nc1C(=O)O] |
| 219a | 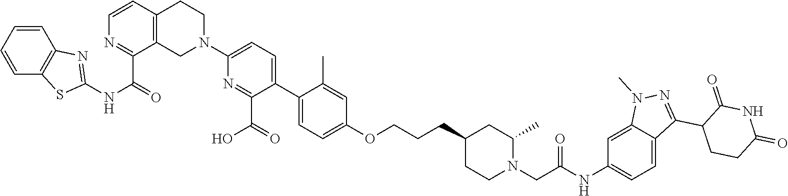 [SMILES: Cc1cc(OCCC[C@@H]2CCN(CC(=O)Nc3ccc4c(C5CCC(=O)NC5=O)nn(C)c4c3)[C@@H](C)C2)ccc1-c1ccc(N2CCc3ccnc(C(=O)Nc4nc5ccccc5s4)c3C2)nc1C(=O)O] |
| 219b | 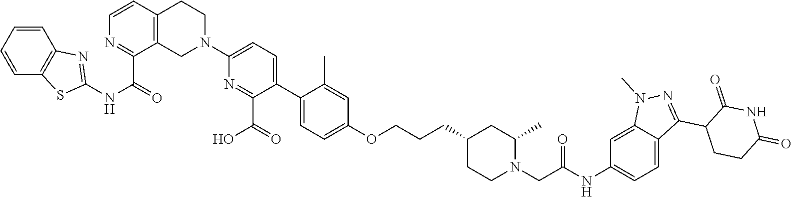 [SMILES: Cc1cc(OCCC[C@H]2CCN(CC(=O)Nc3ccc4c(C5CCC(=O)NC5=O)nn(C)c4c3)[C@@H](C)C2)ccc1-c1ccc(N2CCc3ccnc(C(=O)Nc4nc5ccccc5s4)c3C2)nc1C(=O)O] |
| 219c | 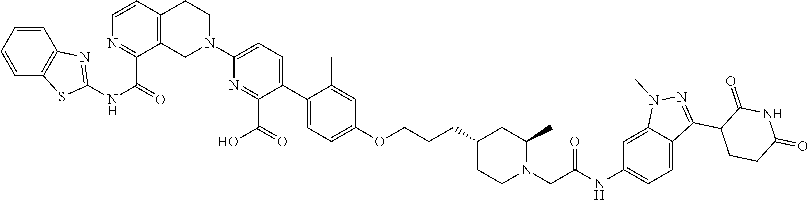 [SMILES: Cc1cc(OCCC[C@H]2CCN(CC(=O)Nc3ccc4c(C5CCC(=O)NC5=O)nn(C)c4c3)[C@H](C)C2)ccc1-c1ccc(N2CCc3ccnc(C(=O)Nc4nc5ccccc5s4)c3C2)nc1C(=O)O] |
| 219d | 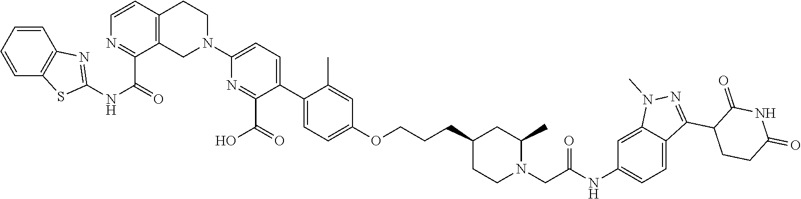 [SMILES: Cc1cc(OCCC[C@@H]2CCN(CC(=O)Nc3ccc4c(C5CCC(=O)NC5=O)nn(C)c4c3)[C@H](C)C2)ccc1-c1ccc(N2CCc3ccnc(C(=O)Nc4nc5ccccc5s4)c3C2)nc1C(=O)O] |
| 220 | 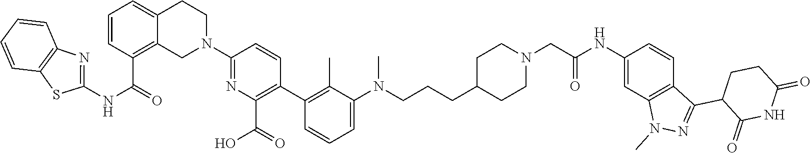 [SMILES: Cc1c(-c2ccc(N3CCc4cccc(C(=O)Nc5nc6ccccc6s5)c4C3)nc2C(=O)O)cccc1N(C)CCCC1CCN(CC(=O)Nc2ccc3c(C4CCC(=O)NC4=O)nn(C)c3c2)CC1] |
| 221 | 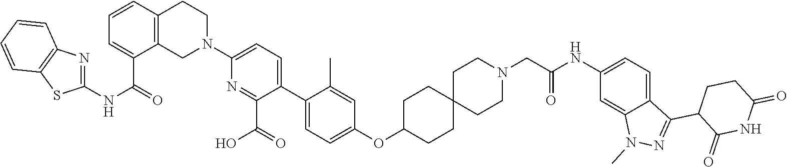 [SMILES: Cc1cc(OC2CCC3(CC2)CCN(CC(=O)Nc2ccc4c(C5CCC(=O)NC5=O)nn(C)c4c2)CC3)ccc1-c1ccc(N2CCc3cccc(C(=O)Nc4nc5ccccc5s4)c3C2)nc1C(=O)O] |
| 222 | 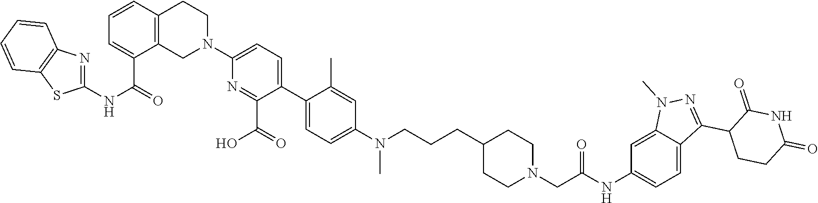 [SMILES: Cc1cc(N(C)CCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 223 | 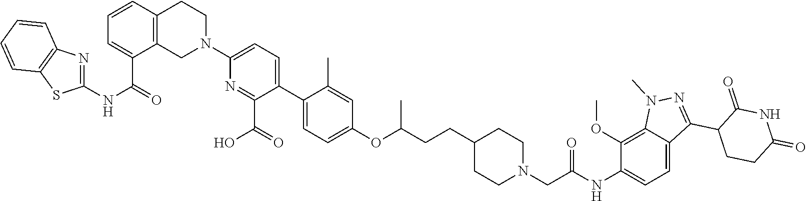 [SMILES: COc1c(NC(=O)CN2CCC(CCC(C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| 223a | 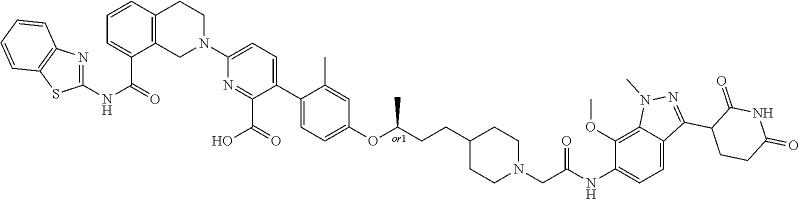 [SMILES: COc1c(NC(=O)CN2CCC(CC[C@H](C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| 223b | 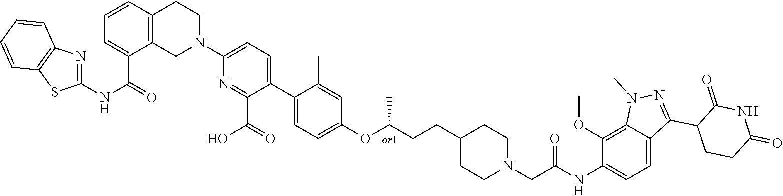 [SMILES: COc1c(NC(=O)CN2CCC(CC[C@@H](C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| 224 | 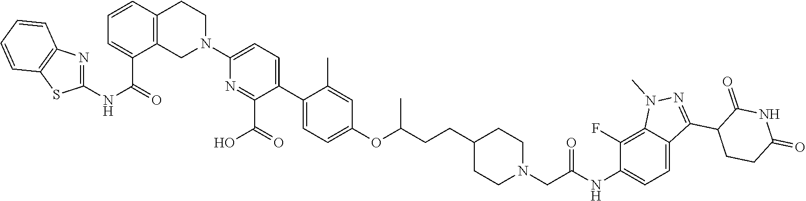 [SMILES: Cc1cc(OC(C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| 224a | 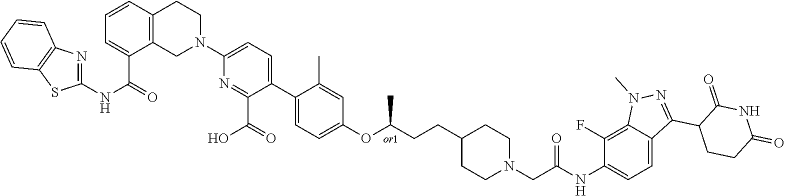 [SMILES: Cc1cc(O[C@@H](C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| 224b | 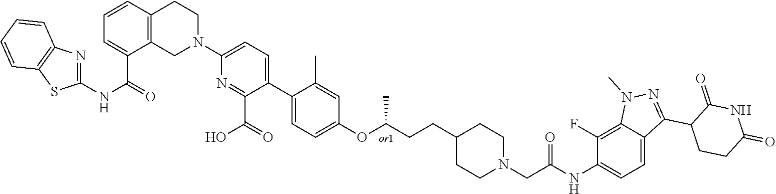 [SMILES: Cc1cc(O[C@H](C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| 225 | 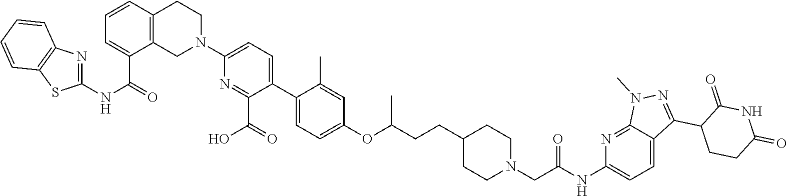 [SMILES: Cc1cc(OC(C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |
| 225a | 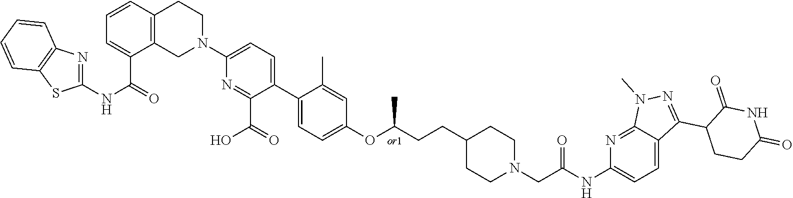 [SMILES: Cc1cc(O[C@@H](C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |
| 225b | 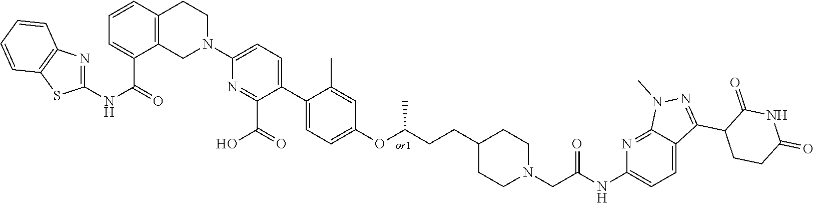 [SMILES: Cc1cc(O[C@H](C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |

Note
for compounds in Table C1, when a stereogenic center is denoted with “or1” in a structural formula, the stereogenic center has been resolved but the configuration
at the stereogenic center has not been determined. For example, the structure 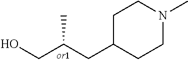
represents (R)-2-methyl-3-(1-methylpiperidin-4-yl)propan-1-ol or (S)-2-methyl-3-(1-methylpiperidin-4-yl)propan-1-ol. For example, the structure 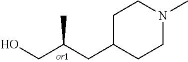
represents (R)-2-methyl-3-(1-methylpiperidin-4-yl)propan-1-ol or (S)-2-methyl-3-(1-methylpiperidin-4-yl)propan-1-ol.

In some embodiments, the compounds of Formula (I) are selected from the group consisting of:

| | |
|---|---|
| 113 | 6-[8-(1,3-benzothiazol-2-ylcarbamoyl)-3,4-dihydro-1H-isoquinolin-2-yl]-3-[3-[[7-[2- |
| | [[3-(2,6-dioxo-3-piperidyl)-1-methyl-indazol-6-yl]amino]-2-oxo-ethyl]-7- |
| | azaspiro[3.5]nonan-2-yl]oxy]-2-methyl-phenyl]pyridine-2-carboxylic acid |
| 133a | 6-(8-(benzo[d]thiazol-2-ylcarbamoyl)-3,4-dihydro-2,7-naphthyridin-2(1H)-yl)-3-(4- |
| | (((2R)-4-(1-(2-((3-(2,6-dioxopiperidin-3-yl)-1-methyl-1H-indazol-6-yl)amino)-2- |
| | oxoethyl)piperidin-4-yl)butan-2-yl)oxy)-2-methylphenyl)picolinic acid |
| 133b | 6-(8-(benzo[d]thiazol-2-ylcarbamoyl)-3,4-dihydro-2,7-naphthyridin-2(1H)-yl)-3-(4- |
| | (((2S)-4-(1-(2-((3-(2,6-dioxopiperidin-3-yl)-1-methyl-1H-indazol-6-yl)amino)-2- |
| | oxoethyl)piperidin-4-yl)butan-2-yl)oxy)-2-methylphenyl)picolinic acid |
| 154 | 6-[8-(1,3-benzothiazol-2-ylcarbamoyl)-3,4-dihydro-1H-2,7-naphthyridin-2-yl]-3-[3-[[7- |
| | [2-[[3-(2,6-dioxo-3-piperidyl)-1-methyl-indazol-6-yl]amino]-2-oxo-ethyl]-7- |
| | azaspiro[3.5]nonan-2-yl]oxy]-2-(trifluoromethyl)phenyl]pyridine-2-carboxylic acid |
| 163 | 6-[8-(1,3-benzothiazol-2-ylcarbamoyl)-3,4-dihydro-1H-2,7-naphthyridin-2-yl]-3-[3-[[7- |
| | [2-[[3-(2,6-dioxo-3-piperidyl)-1-methyl-indazol-6-yl]amino]-2-oxo-ethyl]-7- |
| | azaspiro[3.5]nonan-2-yl]oxy]-2-methyl-phenyl]pyridine-2-carboxylic acid |
| 164 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-7-methoxy-1-methyl-1H-indazol-6-yl)amino)-2-oxoethyl)-7- |
| | azaspiro[3.5]nonan-2-yl)oxy)-2-methylphenyl)picolinic acid |
| 165 | 6-(8-(benzo[d]thiazol-2-ylcarbamoyl)-3,4-dihydro-2,7-naphthyridin-2(1H)-yl)-3-(3-((7- |
| | (2-((3-(2,6-dioxopiperidin-3-yl)-7-methoxy-1-methyl-1H-indazol-6-yl)amino)-2- |
| | oxoethyl)-7-azaspiro[3.5]nonan-2-yl)oxy)-2-(trifluoromethyl)phenyl)picolinic acid |
| 166 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-7-methoxy-1-methyl-1H-indazol-6-yl)amino)-2-oxoethyl)-7- |
| | azaspiro[3.5]nonan-2-yl)oxy)-2-(trifluoromethyl)phenyl)picolinic acid |
| 169 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-1-methyl-1H-indazol-6-yl)amino)-2-oxoethyl)-7- |
| | azaspiro[3.5]nonan-2-yl)oxy)-2-(trifluoromethyl)phenyl)picolinic acid |
| 181 | 6-[8-(1,3-benzothiazol-2-ylcarbamoyl)-3,4-dihydro-1H-2,7-naphthyridin-2-yl]-3-[4-[[7- |
| | [2-[[3-(2,6-dioxo-3-piperidyl)-7-methoxy-1-methyl-indazol-6-yl]amino]-2-oxo-ethyl]-7- |
| | azaspiro[3.5]nonan-2-yl]methoxy]-2-(trifluoromethyl)phenyl]pyridine-2-carboxylic acid |
| 184 | 6-[8-(1,3-benzothiazol-2-ylcarbamoyl)-3,4-dihydro-1H-2,7-naphthyridin-2-yl]-3-[3-[[7- |
| | [2-[[3-(2,6-dioxo-3-piperidyl)-7-methoxy-1-methyl-indazol-6-yl]amino]-2-oxo-ethyl]-7- |
| | azaspiro[3.5]nonan-2-yl]oxy]-2-methyl-phenyl]pyridine-2-carboxylic acid |
| 188 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-7-fluoro-1-methyl-1H-indazol-6-yl)amino)-2-oxoethyl)-7- |
| | azaspiro[3.5]nonan-2-yl)oxy)-2-(trifluoromethyl)phenyl)picolinic acid |
| 190 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-7-fluoro-1-methyl-1H-indazol-6-yl)amino)-2-oxoethyl)-7- |
| | azaspiro[3.5]nonan-2-yl)oxy)-2-methylphenyl)picolinic acid |
| 192 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-1-methyl-1H-pyrazolo[3,4-b]pyridin-6-yl)amino)-2-oxoethyl)- |
| | 7-azaspiro[3.5]nonan-2-yl)oxy)-2-(trifluoromethyl)phenyl)picolinic acid |
| 194 | 6-(8-(benzo[d]thiazol-2-ylcarbamoyl)-3,4-dihydroisoquinolin-2(1H)-yl)-3-(3-((7-(2-((3- |
| | (2,6-dioxopiperidin-3-yl)-1-methyl-1H-pyrazolo[3,4-b]pyridin-6-yl)amino)-2-oxoethyl)- |
| | 7-azaspiro[3.5]nonan-2-yl)oxy)-2-methylphenyl)picolinic acid |
| 202 | 6-(8-(benzo[d]thiazol-2-ylcarbamoyl)-3,4-dihydro-2,7-naphthyridin-2(1H)-yl)-3-(3-((7- |
| | (2-((3-(2,6-dioxopiperidin-3-yl)-1-methyl-1H-pyrazolo[3,4-b]pyridin-6-yl)amino)-2- |
| | oxoethyl)-7-azaspiro[3.5]nonan-2-yl)oxy)-2-(trifluoromethyl)phenyl)picolinic acid |
| 204 | 6-(8-(benzo[d]thiazol-2-ylcarbamoyl)-3,4-dihydro-2,7-naphthyridin-2(1H)-yl)-3-(3-((7- |
| | (2-((3-(2,6-dioxopiperidin-3-yl)-7-fluoro-1-methyl-1H-indazol-6-yl)amino)-2-oxoethyl)- |
| | 7-azaspiro[3.5]nonan-2-yl)oxy)-2-methylphenyl)picolinic acid |
| 207 | 6-[8-(1,3-benzothiazol-2-ylcarbamoyl)-3,4-dihydro-1H-2,7-naphthyridin-2-yl]-3-[3-[[7- |
| | [2-[[3-(2,6-dioxo-3-piperidyl)-1-methyl-pyrazolo[3,4-b]pyridin-6-yl]amino]-2-oxo- |
| | ethyl]-7-azaspiro[3.5]nonan-2-yl]oxy]-2-methyl-phenyl]pyridine-2-carboxylic acid |

or pharmaceutically acceptable salts thereof.

In some embodiments, the compounds of Formula (I) are selected from the group consisting of the compounds depicted in Table C1 of U.S. Provisional Application Ser. No. 63/547,656, filed Nov. 7, 2023; Table C1 of U.S. Provisional Application Ser. No. 63/641,308, filed May 1, 2024; and Table C1 of U.S. Provisional Application Ser. No. 63/704,392, filed Oct. 7, 2024; each of the Table C1s is incorporated by reference in its entirety herein.

In some embodiments, the compounds of Formula (I) are other than the compounds depicted in Tables C1, C2, and/or C3 of International Patent Application No. PCT/US2023/021010, or pharmaceutically acceptable salts thereof.

In some embodiments, the compounds of Formula (I) are other than the compounds depicted in Table R1, or pharmaceutically acceptable salts thereof.

TABLE R1

| No. | Compound Structure |
|---|---|
| R101 | 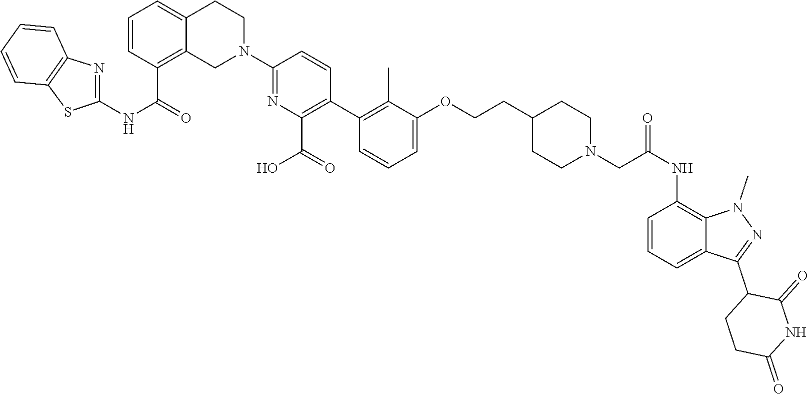 [SMILES: Cc1c(OCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R103 | 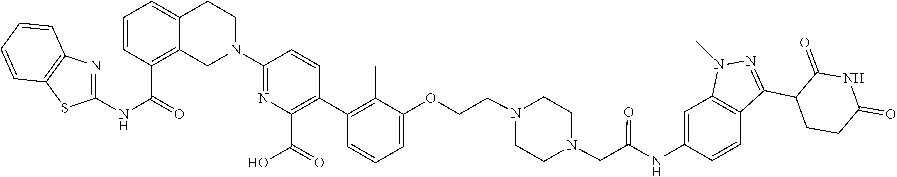 [SMILES: Cc1c(OCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R104 | 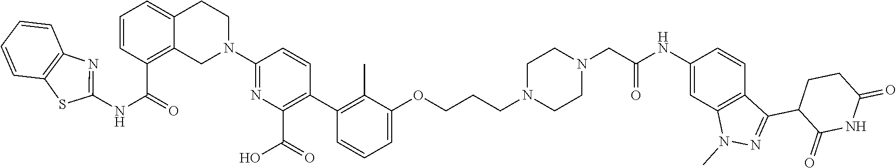 [SMILES: Cc1c(OCCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R105 | 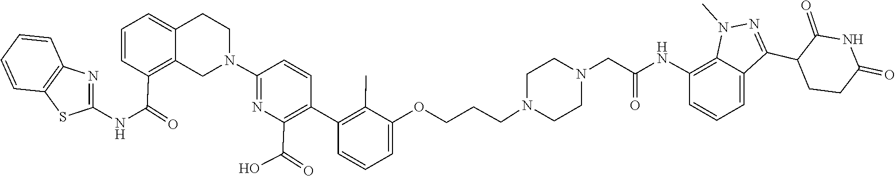 [SMILES: Cc1c(OCCCN2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R106 | 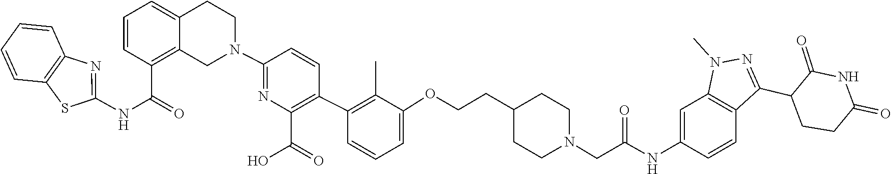 [SMILES: Cc1c(OCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R107 | 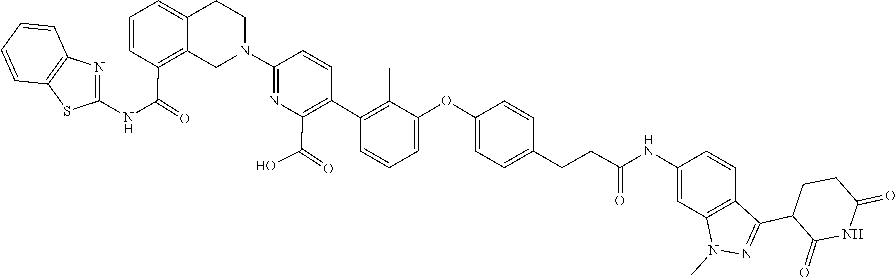 [SMILES: Cc1c(Oc2ccc(CCC(=O)Nc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O] |
| R116 | 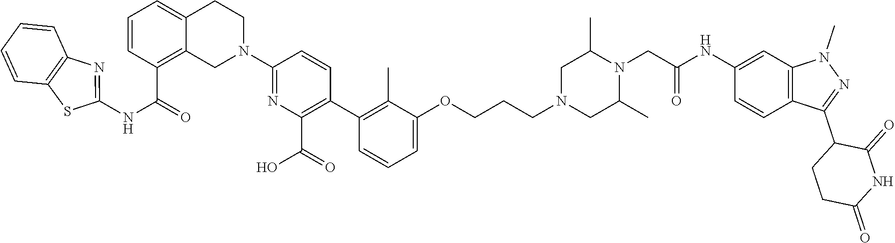 [SMILES: Cc1c(OCCCN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| R116a | 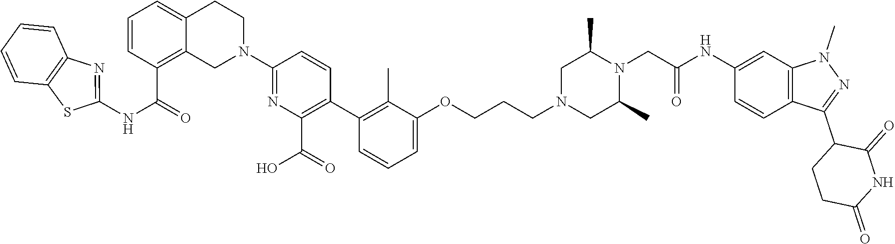 [SMILES: Cc1c(OCCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| R117 | 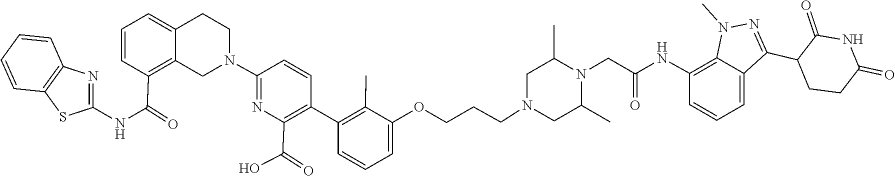 [SMILES: Cc1c(OCCCN2CC(C)N(CC(=O)Nc3cccc4c(C5CCC(=O)NC5=O)nn(C)c34)C(C)C2)cccc1-c1ccc(N2CCc3cccc(C(=O)Nc4nc5ccccc5s4)c3C2)nc1C(=O)O] |
| R117a | 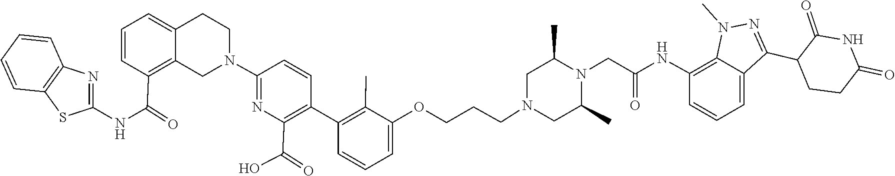 [SMILES: Cc1c(OCCCN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O] |
| R119 | 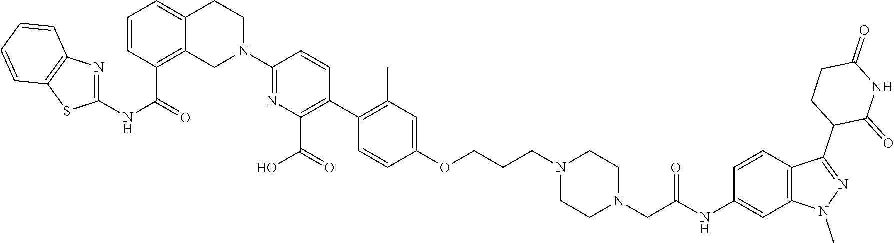 [SMILES: Cc1cc(OCCCN2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R120 | 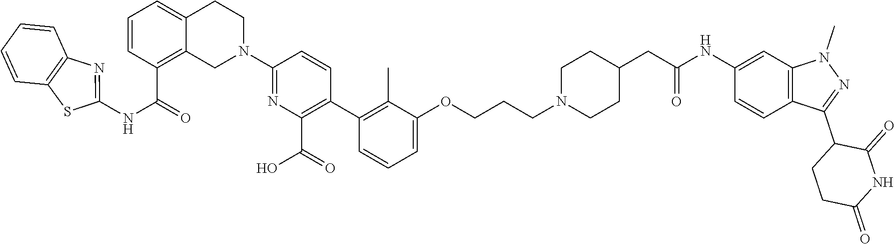 [SMILES: Cc1c(OCCCN2CCC(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R121 | 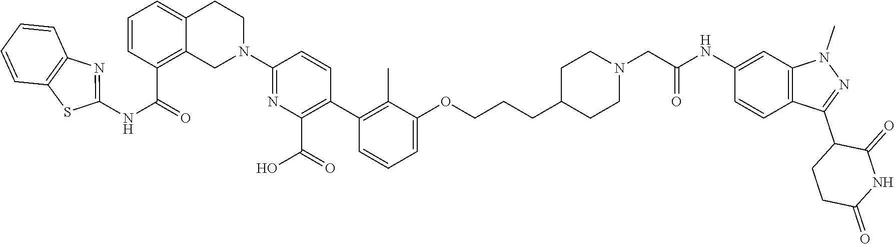 [SMILES: Cc1c(OCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R122 | 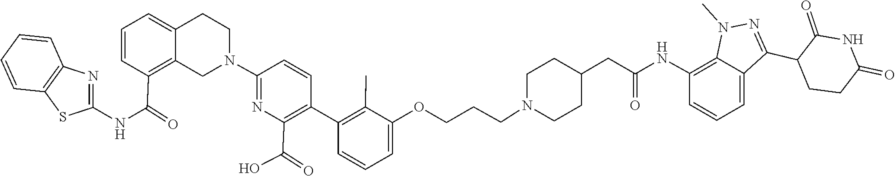 [SMILES: Cc1c(OCCCN2CCC(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R123 | 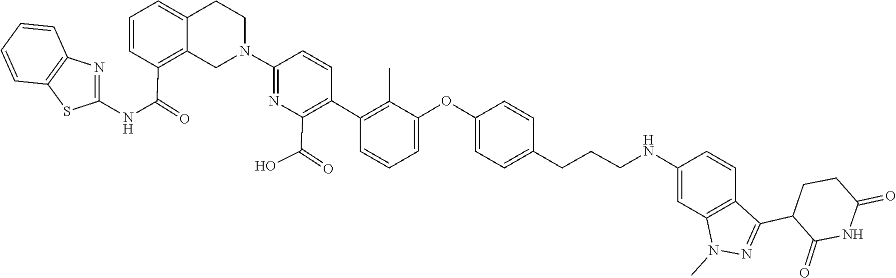 [SMILES: Cc1c(Oc2ccc(CCCNc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O] |
| R124 | 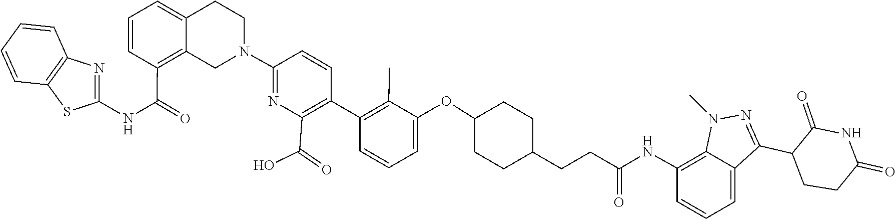 [SMILES: Cc1c(OC2CCC(CCC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R124a | 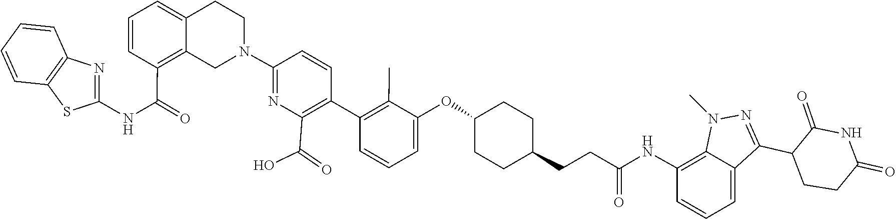 [SMILES: Cc1c(O[C@H]2CC[C@H](CCC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R125 | 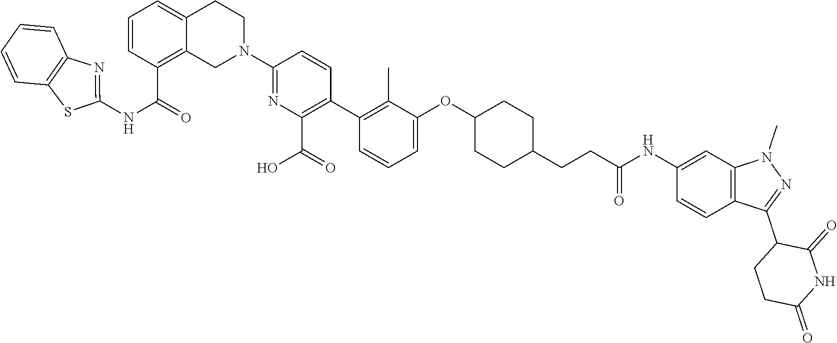 [SMILES: Cc1c(OC2CCC(CCC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R125a | 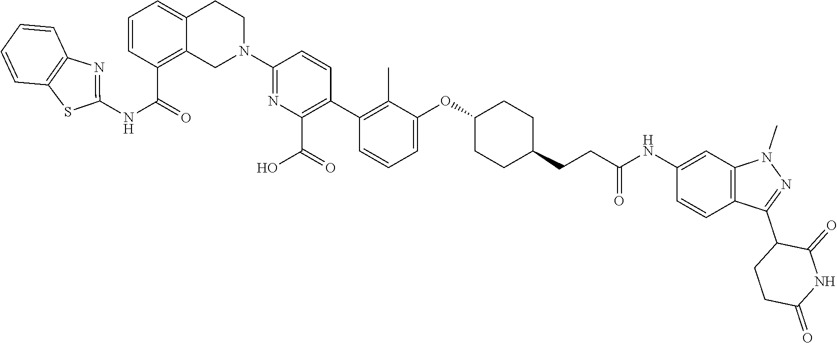 [SMILES: Cc1c(O[C@H]2CC[C@H](CCC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R126 | 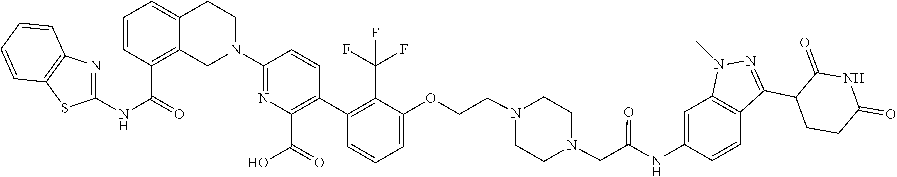 [SMILES: Cn1nc(C2CCC(=O)NC2=O)c2ccc(NC(=O)CN3CCN(CCOc4cccc(-c5ccc(N6CCc7cccc(C(=O)Nc8nc9ccccc9s8)c7C6)nc5C(=O)O)c4C(F)(F)F)CC3)cc21] |
| R127 | 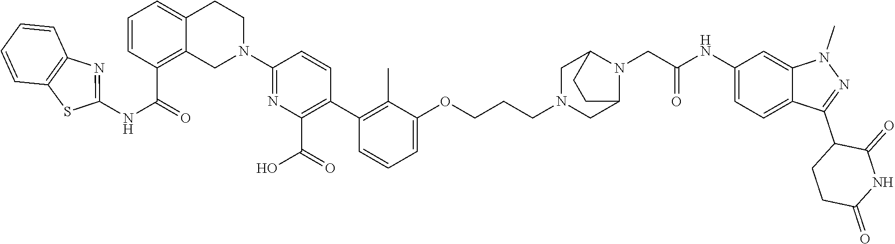 [SMILES: Cc1c(OCCCN2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| R128 | 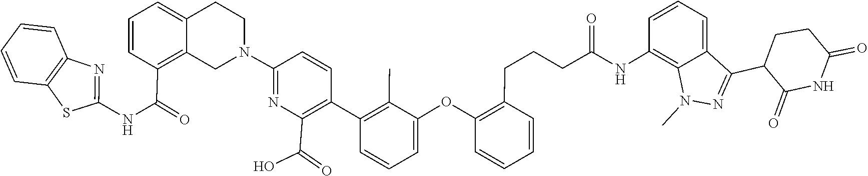 [SMILES: Cc1c(Oc2ccccc2CCCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| R129 | 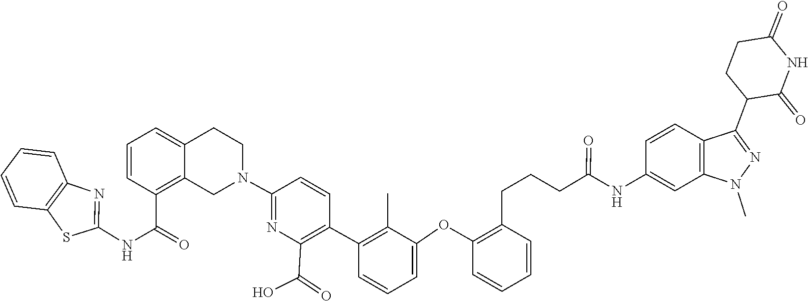 [SMILES: Cc1c(Oc2ccccc2CCCC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| R130 | 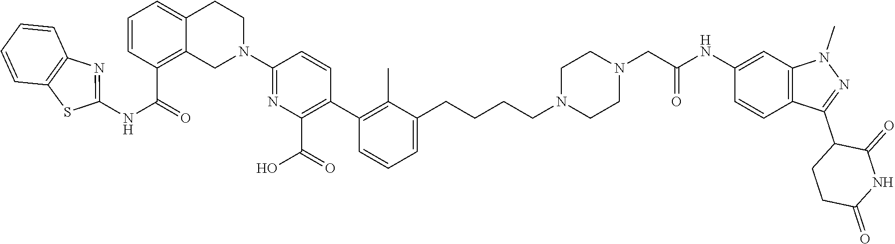 [SMILES: Cc1c(CCCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R131 | 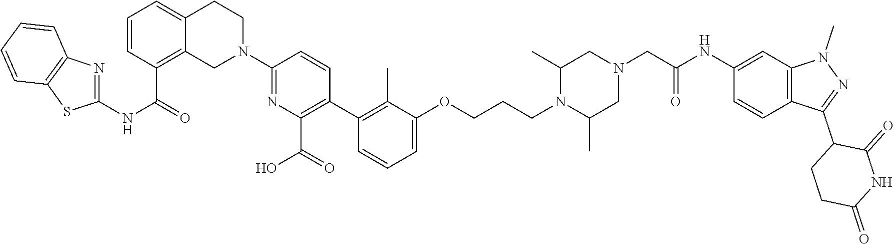 [SMILES: Cc1c(OCCCN2C(C)CN(CC(=O)Nc3ccc4c(C5CCC(=O)NC5=O)nn(C)c4c3)CC2C)cccc1-c1ccc(N2CCc3cccc(C(=O)Nc4nc5ccccc5s4)c3C2)nc1C(=O)O] |
| R131a | 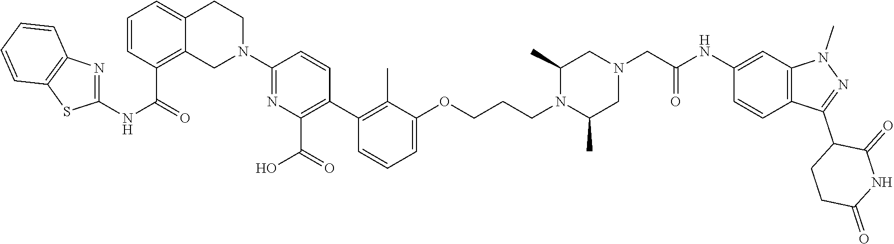 [SMILES: Cc1c(OCCCN2[C@H](C)CN(CC(=O)Nc3ccc4c(C5CCC(=O)NC5=O)nn(C)c4c3)C[C@@H]2C)cccc1-c1ccc(N2CCc3cccc(C(=O)Nc4nc5ccccc5s4)c3C2)nc1C(=O)O] |
| R132 | 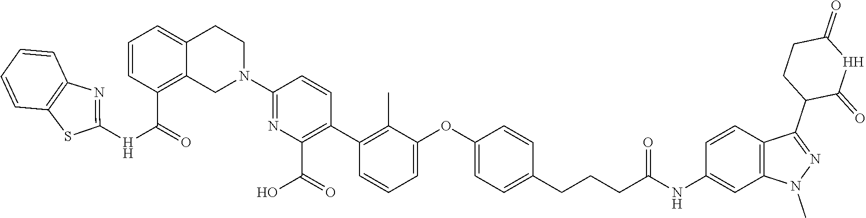 |
| R133 | 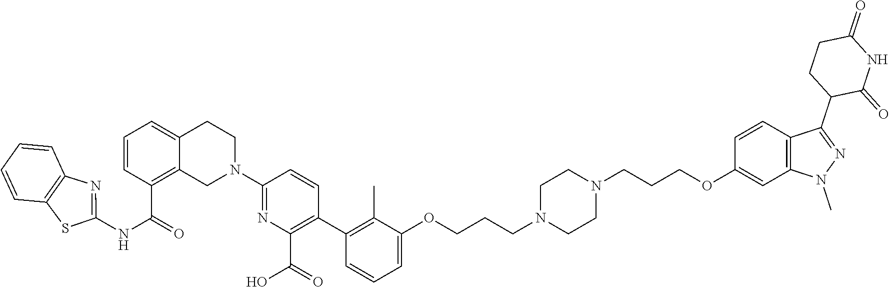 [SMILES: Cc1c(OCCCN2CCN(CCCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R136 | 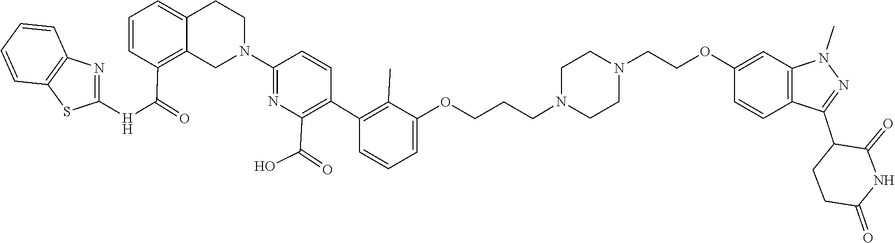 |
| R137 | 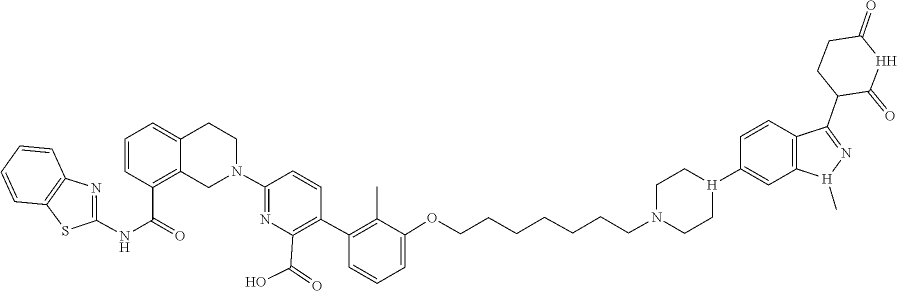 |
| R139 | 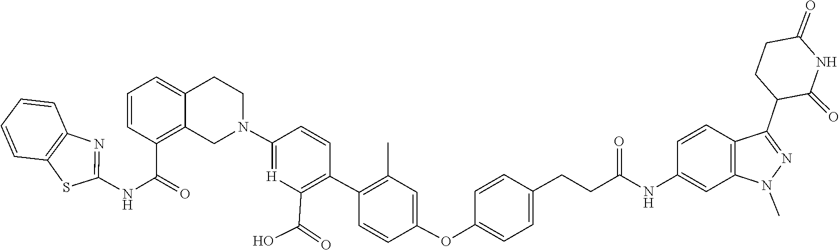 |
| R156 | 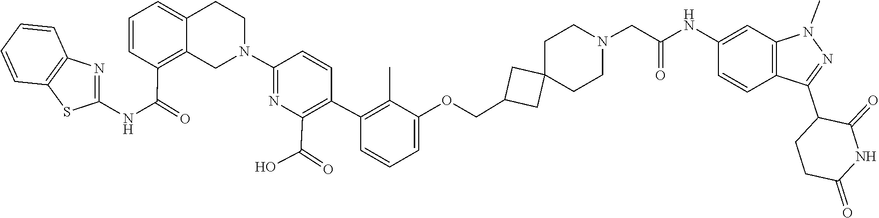 [SMILES: Cc1c(OCC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R158 | 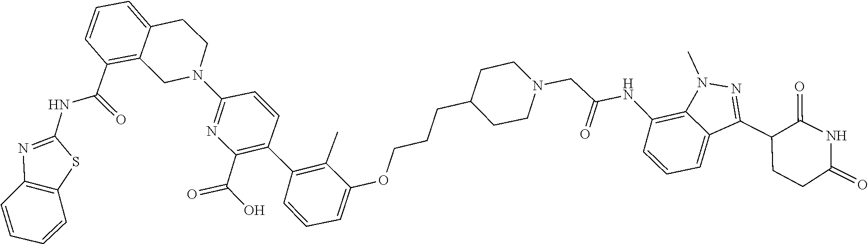 [SMILES: Cc1c(OCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R159 | 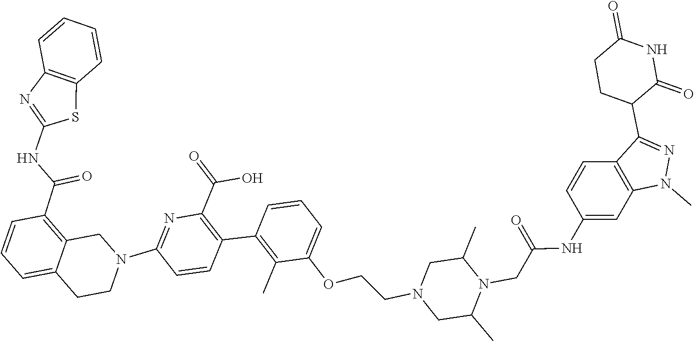 [SMILES: Cc1c(OCCN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| R159a | 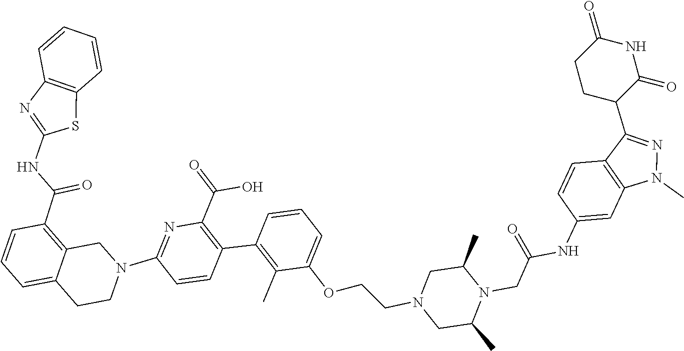 [SMILES: Cc1c(OCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| R160 | 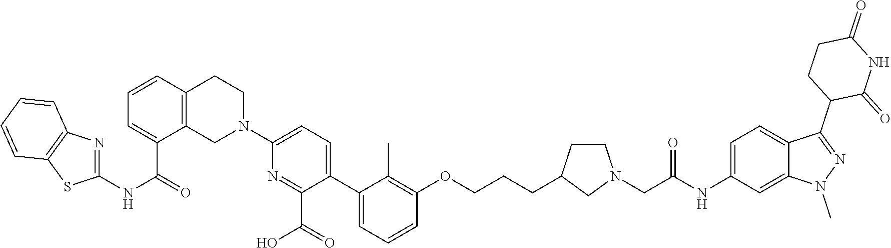 [SMILES: Cc1c(OCCCC2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R160a | 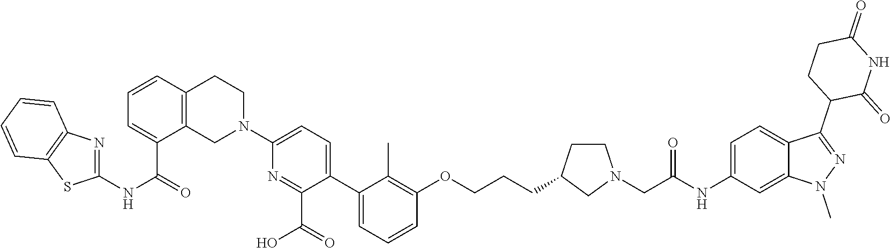 [SMILES: Cc1c(OCCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R160b | 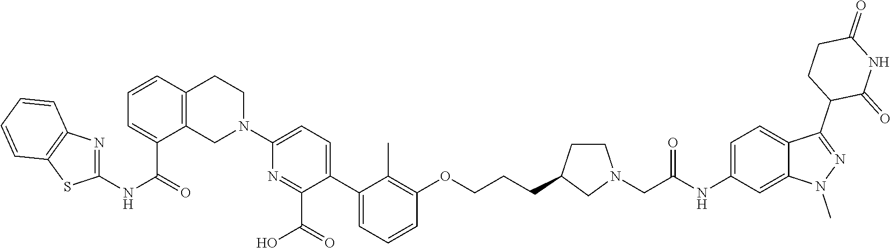 [SMILES: Cc1c(OCCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R162 | 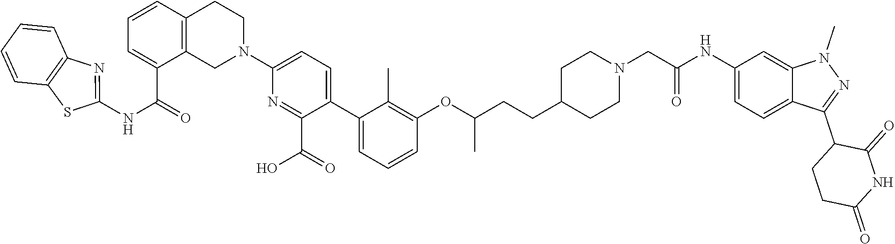 [SMILES: Cc1c(OC(C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R162a | 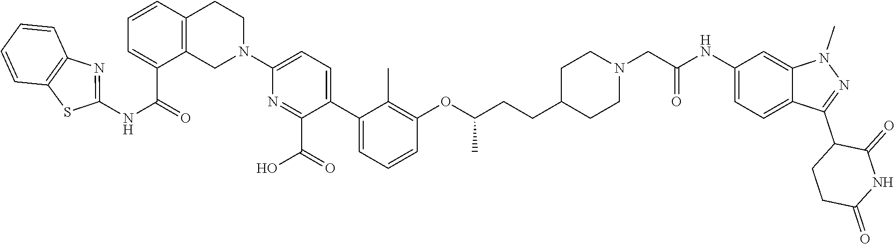 [SMILES: Cc1c(O[C@@H](C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R162b | 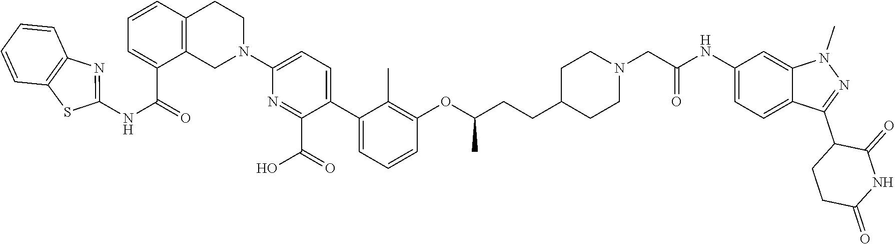 [SMILES: Cc1c(O[C@H](C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R164 | 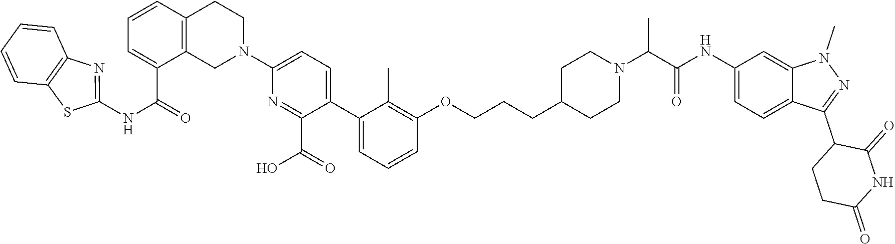 [SMILES: Cc1c(OCCCC2CCN(C(C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R164a | 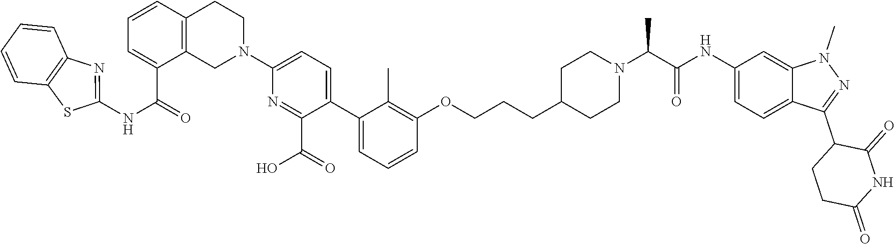 [SMILES: Cc1c(OCCCC2CCN([C@@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R164b | 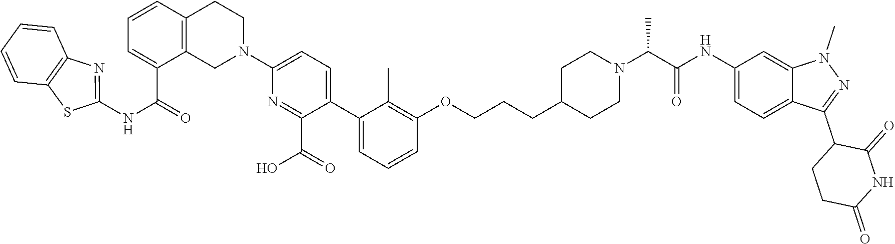 [SMILES: Cc1c(OCCCC2CCN([C@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R165 | 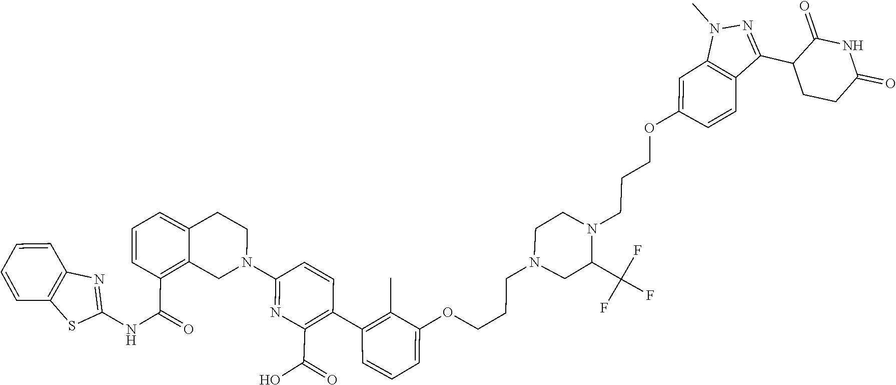 [SMILES: Cc1c(OCCCN2CCN(CCCOc3ccc4c(C5CCC(=O)NC5=O)nn(C)c4c3)C(C(F)(F)F)C2)cccc1-c1ccc(N2CCc3cccc(C(=O)Nc4nc5ccccc5s4)c3C2)nc1C(=O)O] |
| R166 | 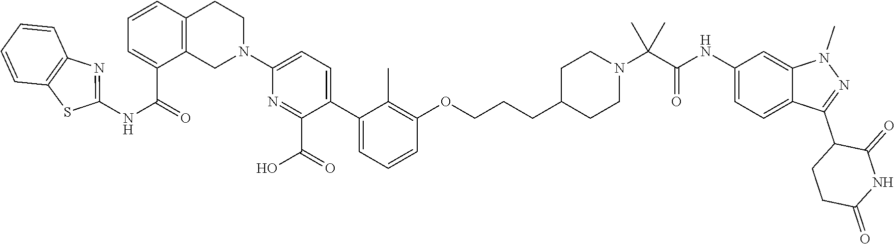 [SMILES: Cc1c(OCCCC2CCN(C(C)(C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R167 | 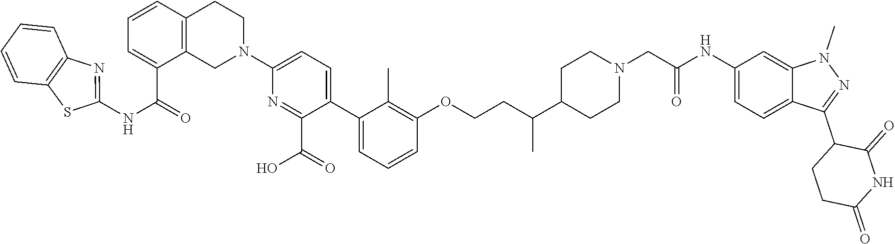 [SMILES: Cc1c(OCCC(C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R167a | 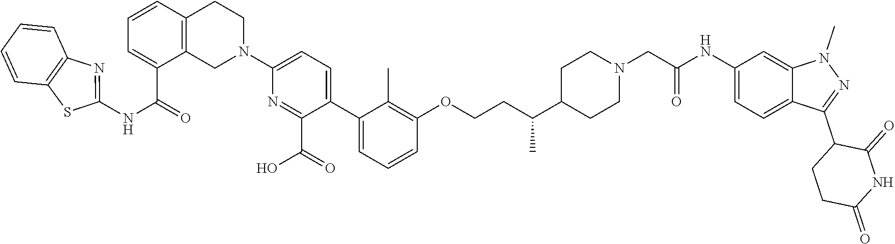 [SMILES: Cc1c(OCC[C@@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R167b | 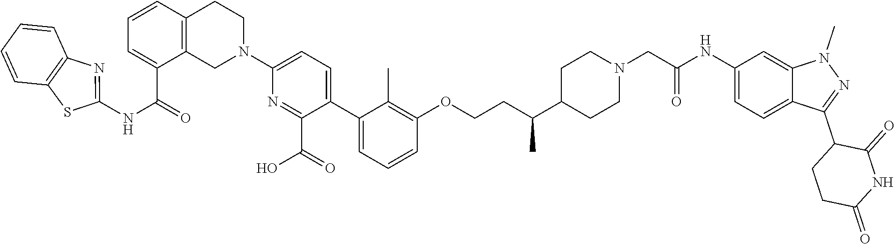 [SMILES: Cc1c(OCC[C@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R169 | 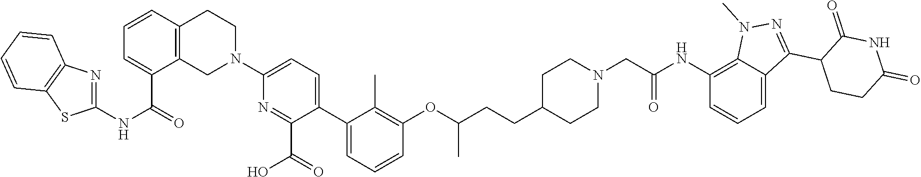 [SMILES: Cc1c(OC(C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R169a | 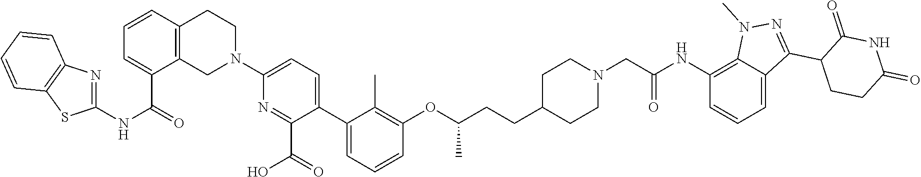 [SMILES: Cc1c(O[C@@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R169b | 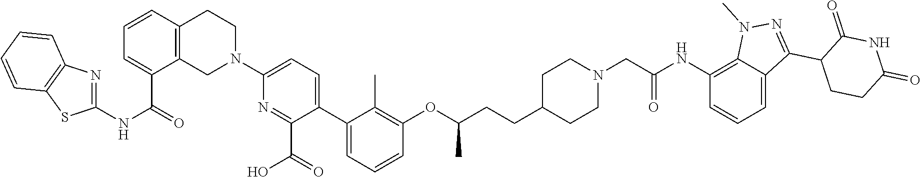 [SMILES: Cc1c(O[C@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R170 | 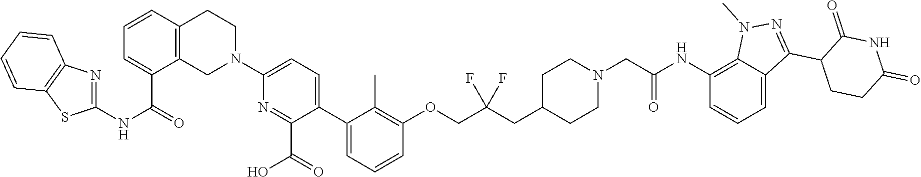 [SMILES: Cc1c(OCC(F)(F)CC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R172 | 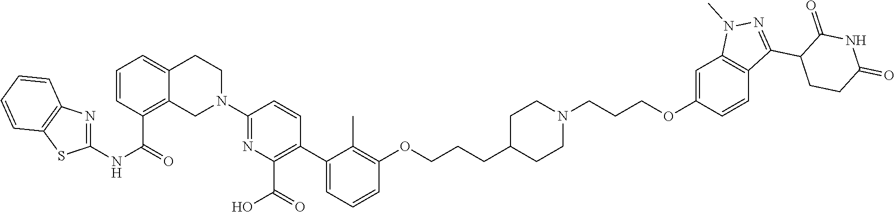 [SMILES: Cc1c(OCCCC2CCN(CCCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R173 | 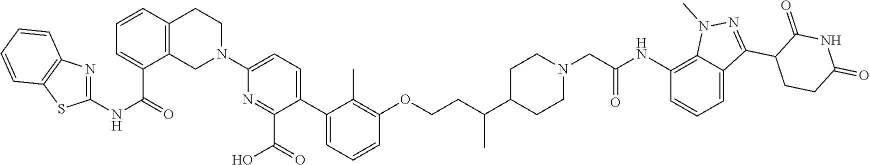 [SMILES: Cc1c(OCCC(C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R173a | 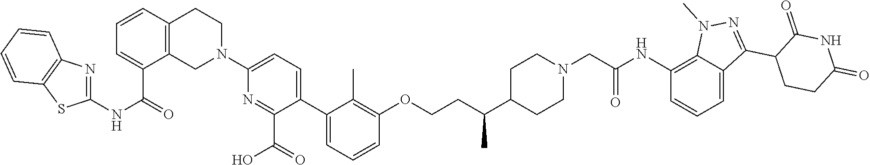 [SMILES: Cc1c(OCC[C@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R173b | 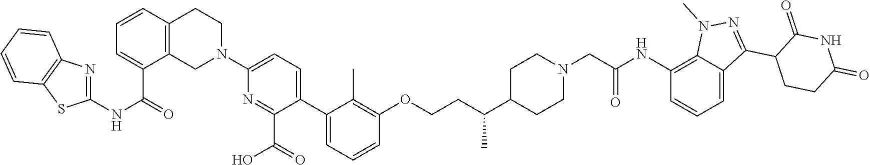 [SMILES: Cc1c(OCC[C@@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R177 | 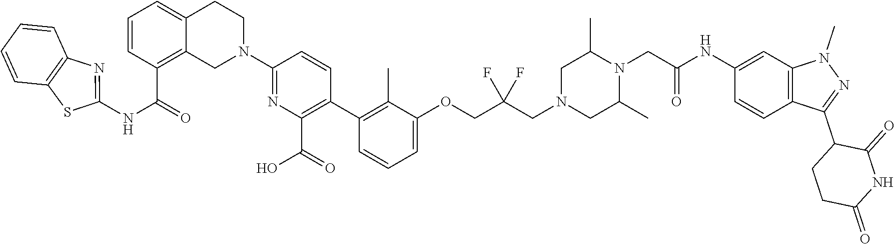 [SMILES: Cc1c(OCC(F)(F)CN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| R177a | 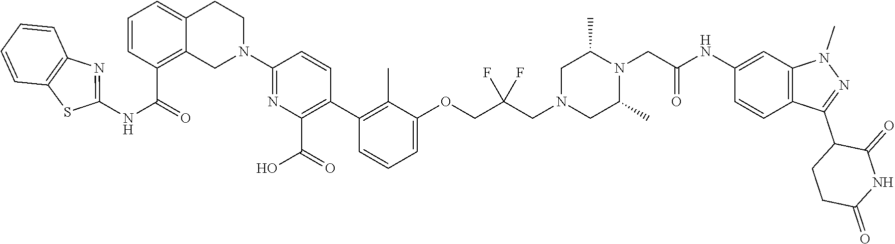 [SMILES: Cc1c(OCC(F)(F)CN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| R180 | 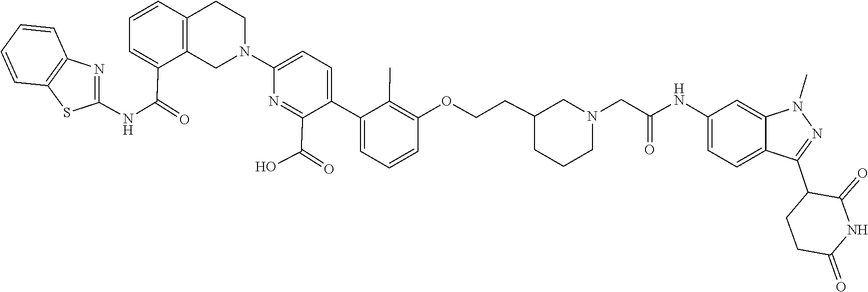 [SMILES: Cc1c(OCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R180a | 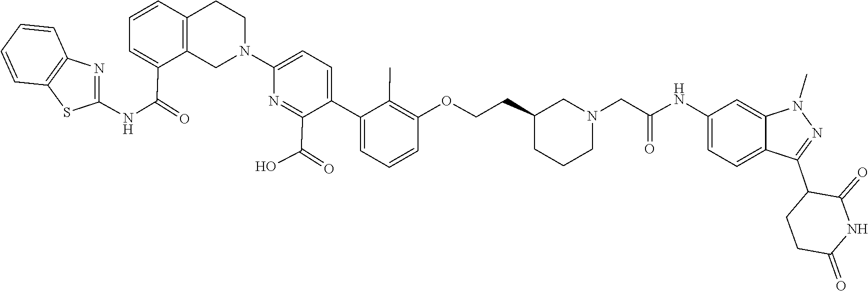 [SMILES: Cc1c(OCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R180b | 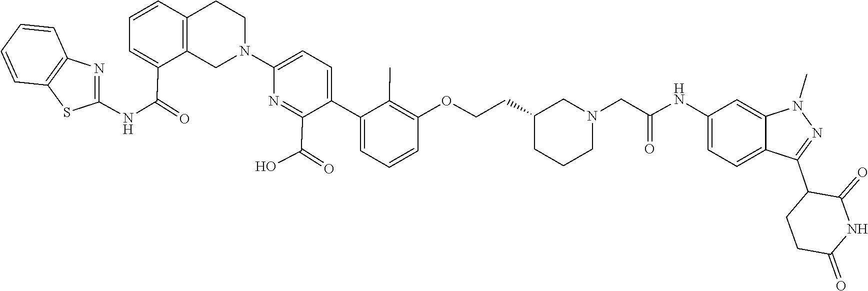 [SMILES: Cc1c(OCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R181 | 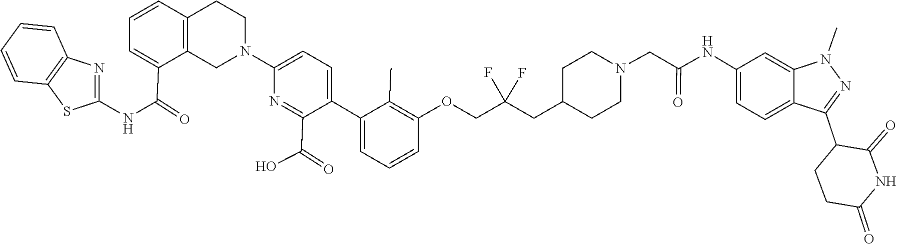 [SMILES: Cc1c(OCC(F)(F)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R184 | 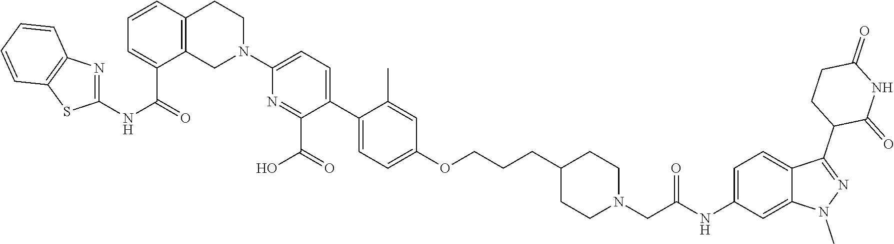 [SMILES: Cc1cc(OCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R186 | 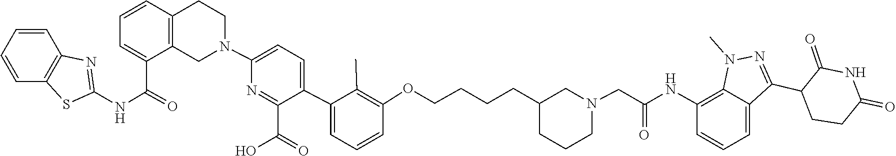 [SMILES: Cc1c(OCCCCC2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R186a | 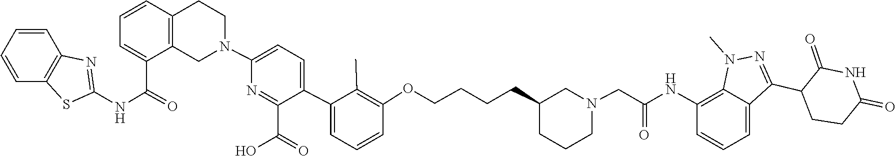 [SMILES: Cc1c(OCCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R186b | 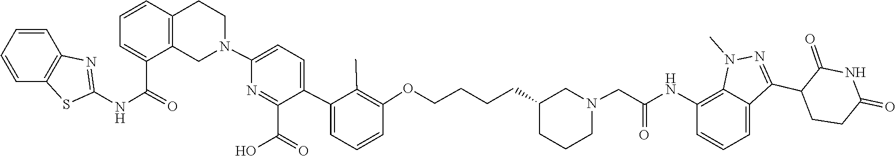 [SMILES: Cc1c(OCCCC[C@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R187 | 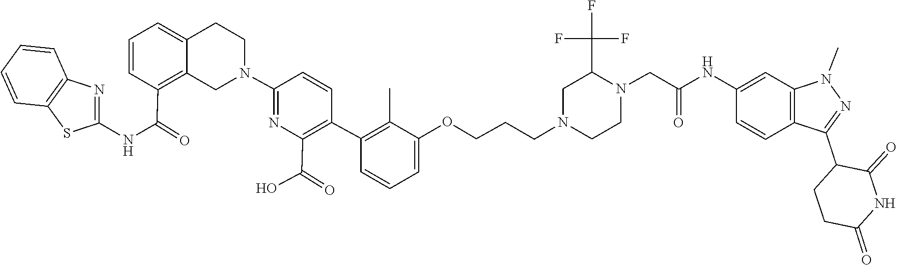 [SMILES: Cc1c(OCCCN2CCN(CC(=O)Nc3ccc4c(C5CCC(=O)NC5=O)nn(C)c4c3)C(C(F)(F)F)C2)cccc1-c1ccc(N2CCc3cccc(C(=O)Nc4nc5ccccc5s4)c3C2)nc1C(=O)O] |
| R187a | 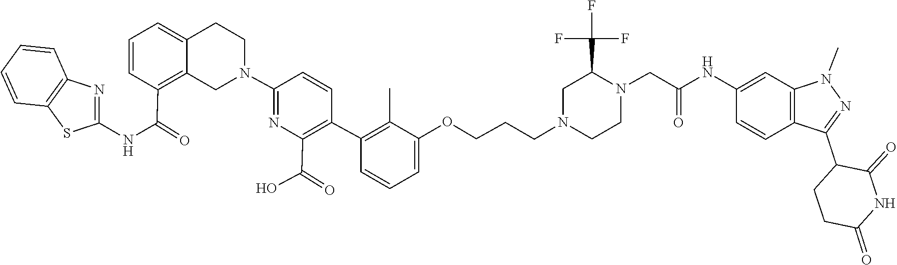 [SMILES: Cc1c(OCCCN2CCN(CC(=O)Nc3ccc4c(C5CCC(=O)NC5=O)nn(C)c4c3)[C@H](C(F)(F)F)C2)cccc1-c1ccc(N2CCc3cccc(C(=O)Nc4nc5ccccc5s4)c3C2)nc1C(=O)O] |
| R187b | 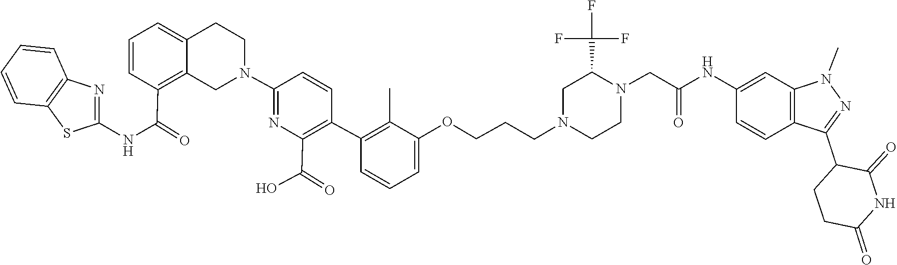 [SMILES: Cc1c(OCCCN2CCN(CC(=O)Nc3ccc4c(C5CCC(=O)NC5=O)nn(C)c4c3)[C@@H](C(F)(F)F)C2)cccc1-c1ccc(N2CCc3cccc(C(=O)Nc4nc5ccccc5s4)c3C2)nc1C(=O)O] |
| R193 | 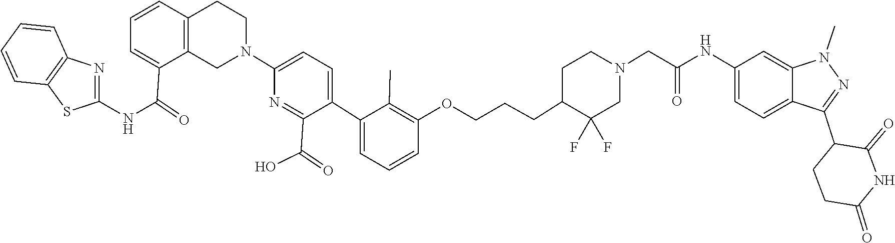 [SMILES: Cc1c(OCCCC2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| R193a | 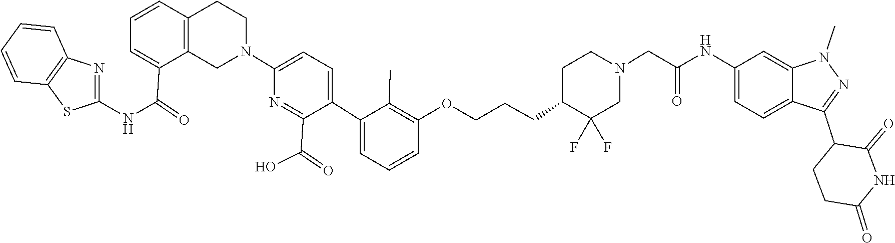 [SMILES: Cc1c(OCCC[C@@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| R193b | 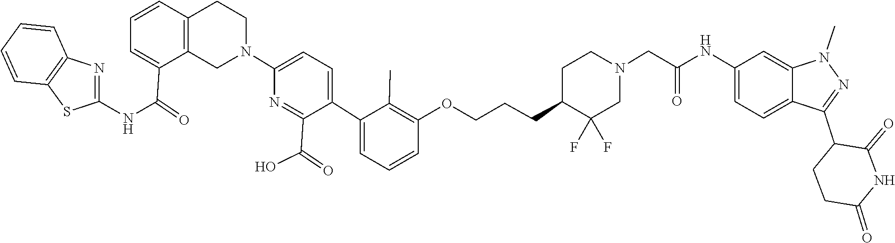 [SMILES: Cc1c(OCCC[C@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| R196 | 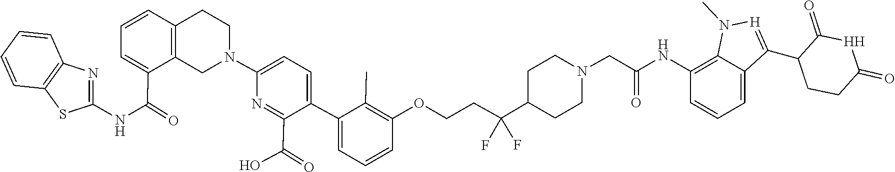 |
| R197 | 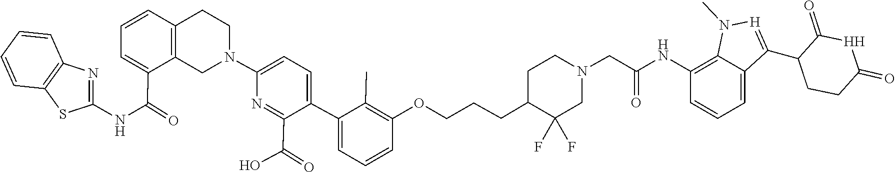 |
| R197a | 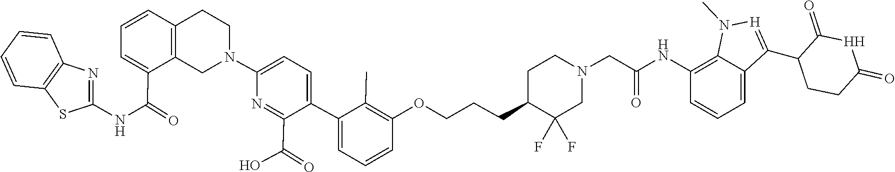 |
| R197b | 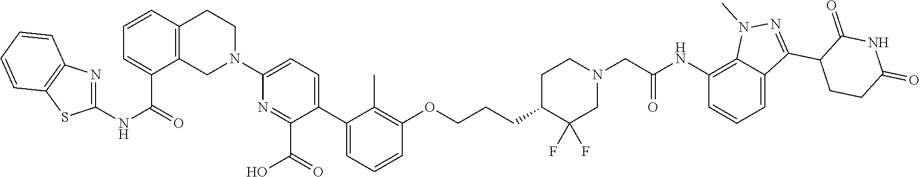 [SMILES: Cc1c(OCCC[C@@H]2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O] |
| R202 | 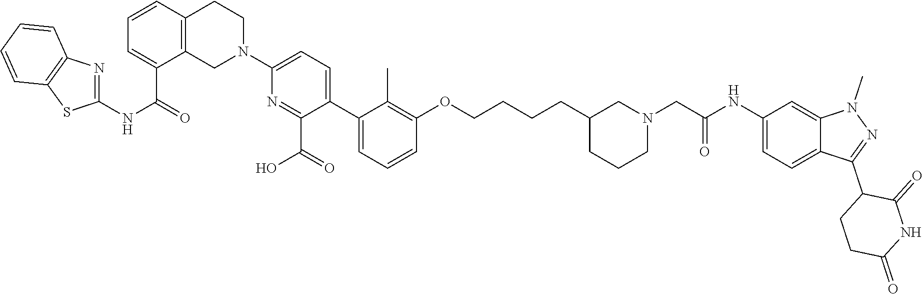 [SMILES: Cc1c(OCCCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R202a | 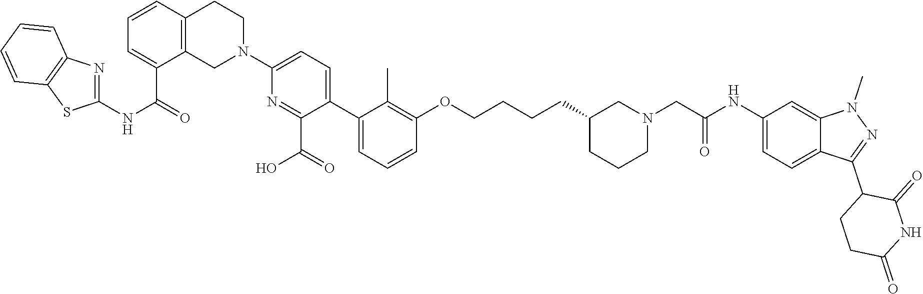 [SMILES: Cc1c(OCCCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R202b | 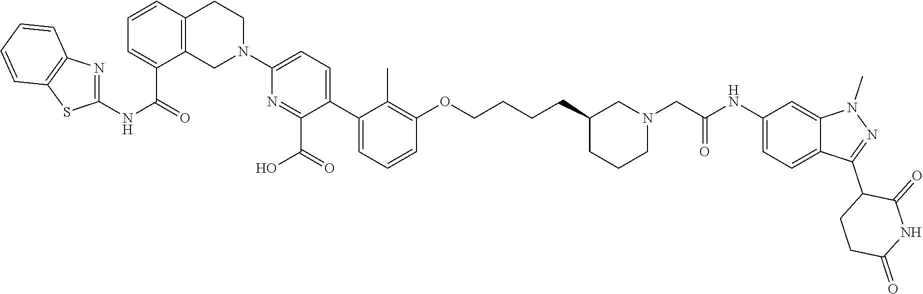 [SMILES: Cc1c(OCCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R203 | 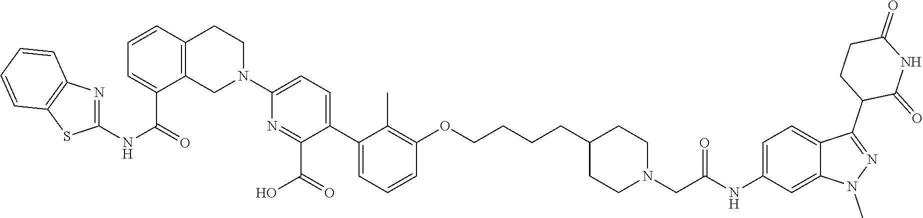 [SMILES: Cc1c(OCCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R205 | 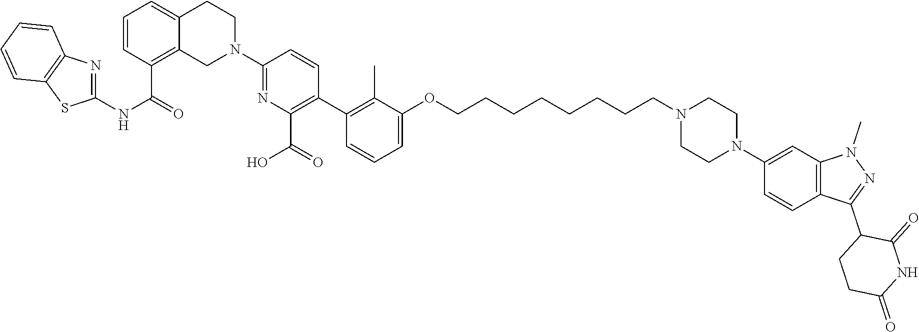 [SMILES: Cc1c(OCCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R206 | 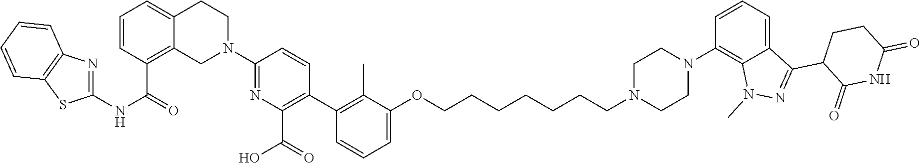 [SMILES: Cc1c(OCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R207 | 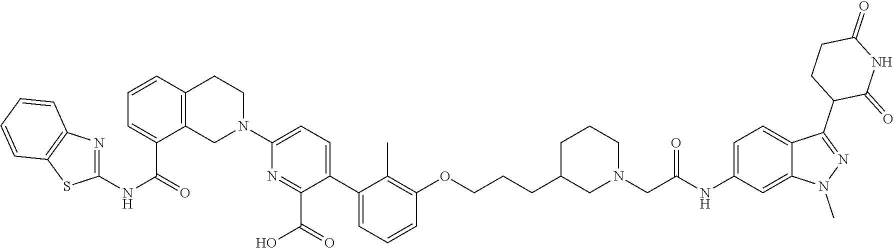 [SMILES: Cc1c(OCCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R207a | 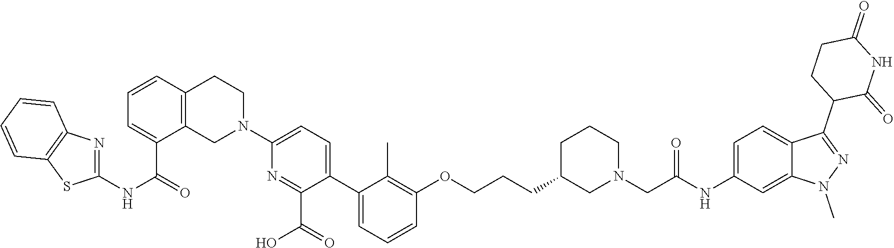 [SMILES: Cc1c(OCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R207b | 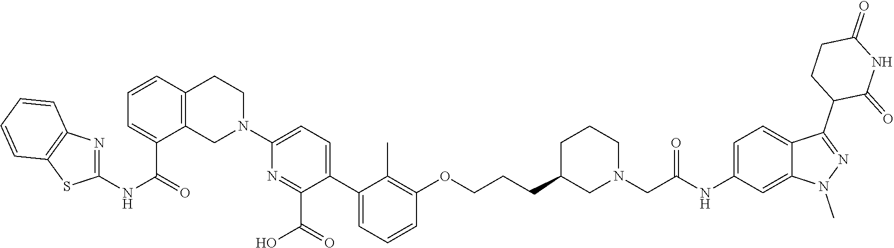 [SMILES: Cc1c(OCCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R209 | 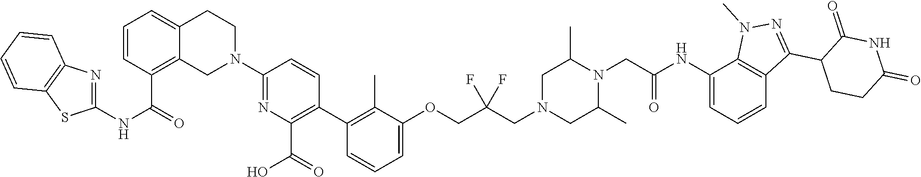 [SMILES: Cc1c(OCC(F)(F)CN2CC(C)N(CC(=O)Nc3cccc4c(C5CCC(=O)NC5=O)nn(C)c34)C(C)C2)cccc1-c1ccc(N2CCc3cccc(C(=O)Nc4nc5ccccc5s4)c3C2)nc1C(=O)O] |
| R209a | 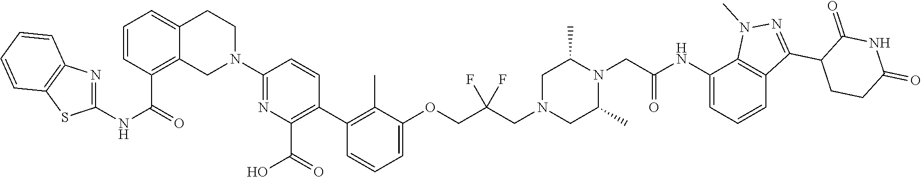 [SMILES: Cc1c(OCC(F)(F)CN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O] |
| R210 | 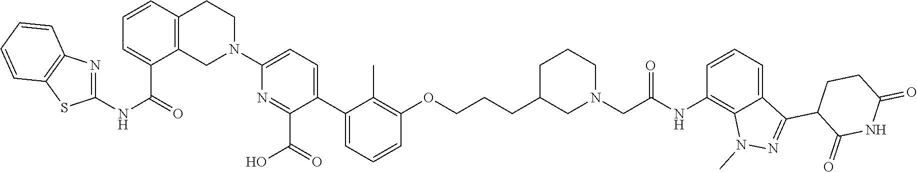 [SMILES: Cc1c(OCCCC2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R210a | 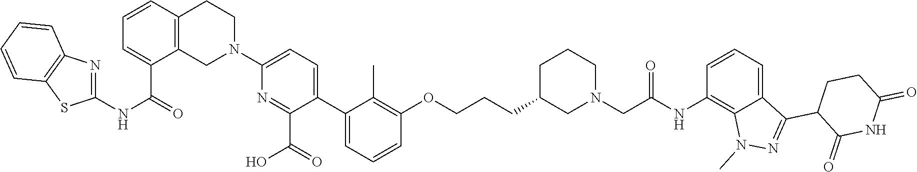 [SMILES: Cc1c(OCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R210b | 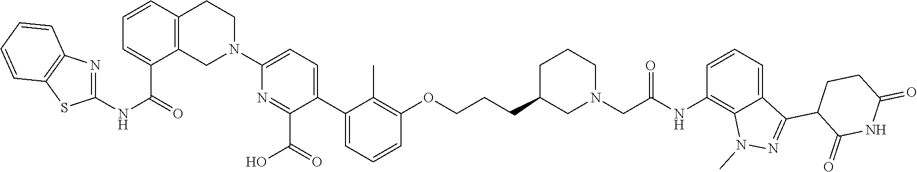 [SMILES: Cc1c(OCCC[C@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R213 | 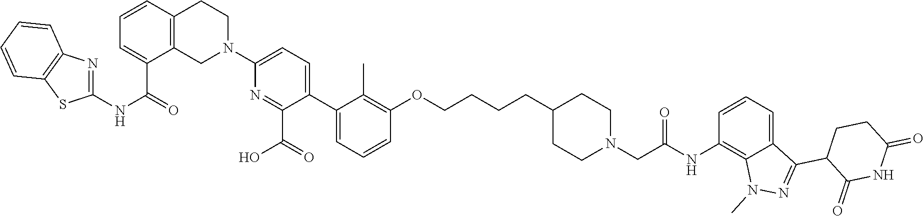 [SMILES: Cc1c(OCCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R216 | 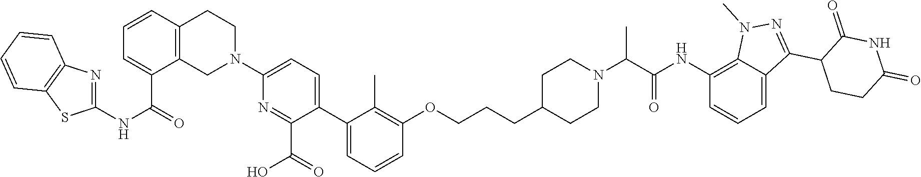 [SMILES: Cc1c(OCCCC2CCN(C(C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R216a | 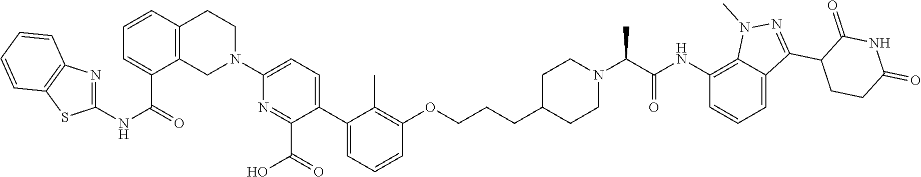 [SMILES: Cc1c(OCCCC2CCN([C@@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R216b | 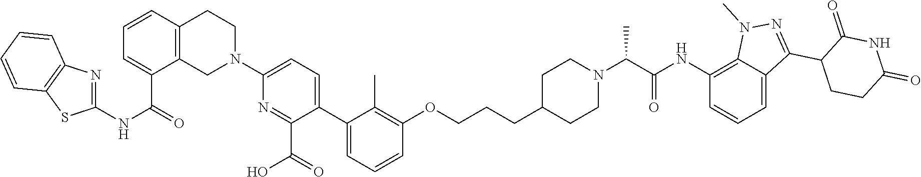 [SMILES: Cc1c(OCCCC2CCN([C@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R217 | 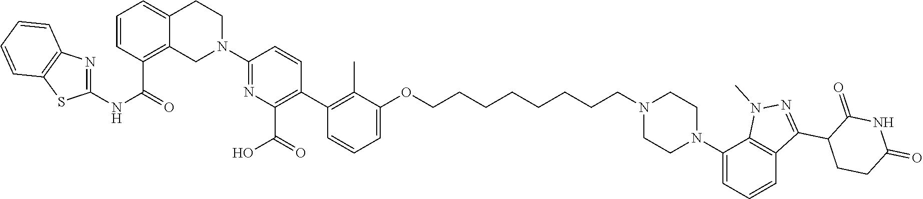 [SMILES: Cc1c(OCCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| R218 | 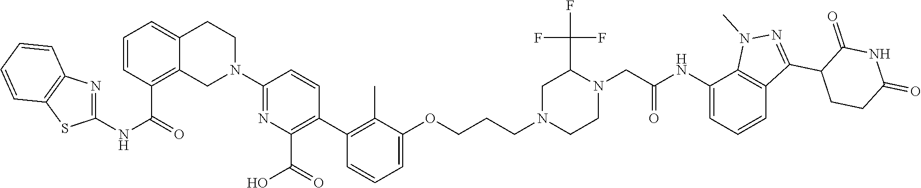 [SMILES: Cc1c(OCCCN2CCN(CC(=O)Nc3cccc4c(C5CCC(=O)NC5=O)nn(C)c34)C(C(F)(F)F)C2)cccc1-c1ccc(N2CCc3cccc(C(=O)Nc4nc5ccccc5s4)c3C2)nc1C(=O)O] |
| R218a | 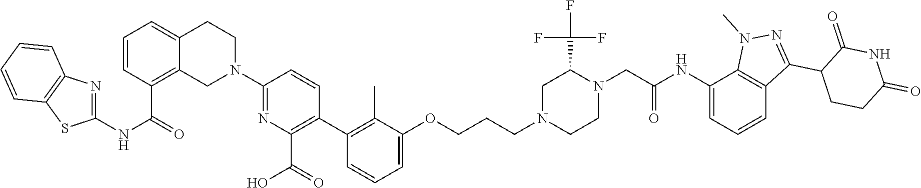 [SMILES: Cc1c(OCCCN2CCN(CC(=O)Nc3cccc4c(C5CCC(=O)NC5=O)nn(C)c34)[C@@H](C(F)(F)F)C2)cccc1-c1ccc(N2CCc3cccc(C(=O)Nc4nc5ccccc5s4)c3C2)nc1C(=O)O] |
| R218b | 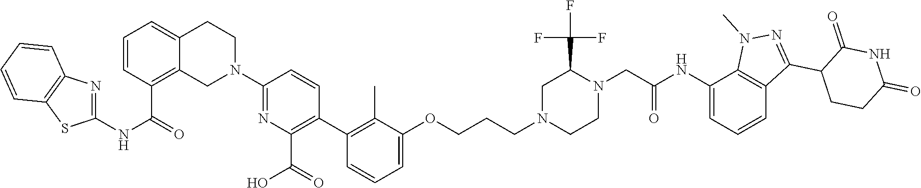 [SMILES: Cc1c(OCCCN2CCN(CC(=O)Nc3cccc4c(C5CCC(=O)NC5=O)nn(C)c34)[C@H](C(F)(F)F)C2)cccc1-c1ccc(N2CCc3cccc(C(=O)Nc4nc5ccccc5s4)c3C2)nc1C(=O)O] |
| R219 | 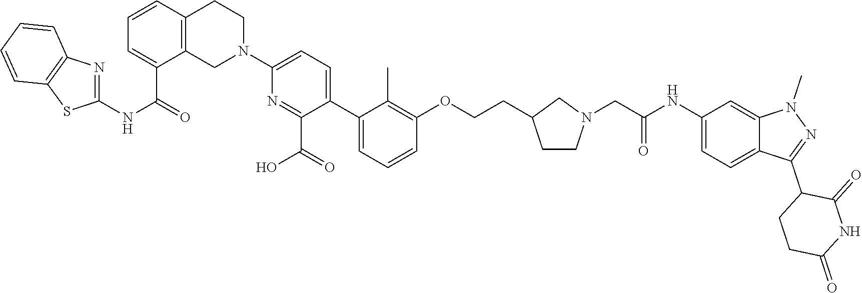 [SMILES: Cc1c(OCCC2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R219a | 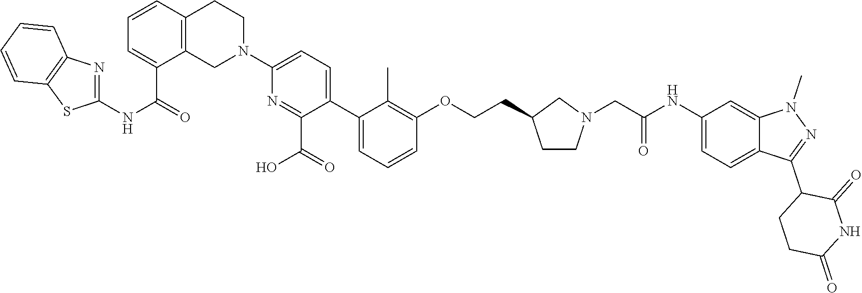 [SMILES: Cc1c(OCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R219b | 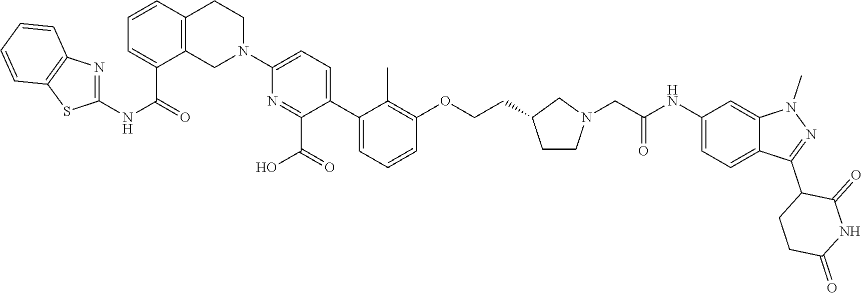 [SMILES: Cc1c(OCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| R222 | 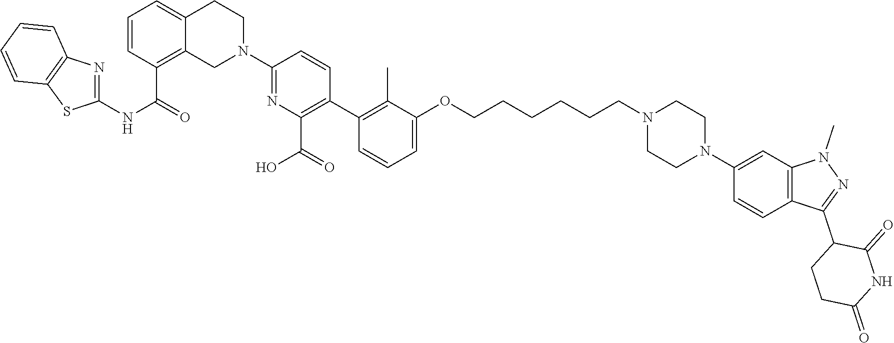 [SMILES: Cc1c(OCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R226 | 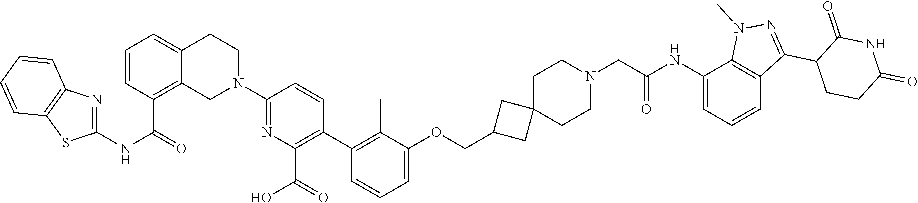 [SMILES: Cc1c(OCC2CC3(CCN(CC(=O)Nc4cccc5c(C6CCC(=O)NC6=O)nn(C)c45)CC3)C2)cccc1-c1ccc(N2CCc3cccc(C(=O)Nc4nc5ccccc5s4)c3C2)nc1C(=O)O] |
| R229 | 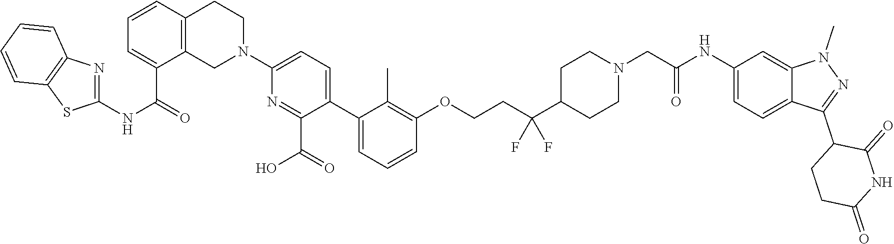 [SMILES: Cc1c(OCCC(F)(F)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R231 | 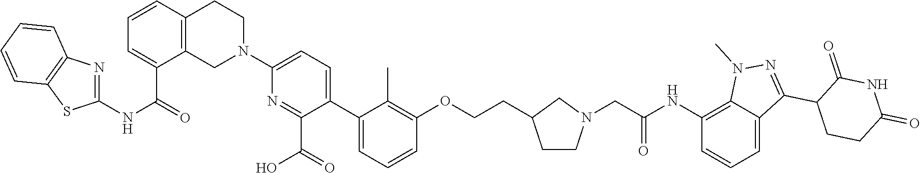 [SMILES: Cc1c(OCCC2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R231a | 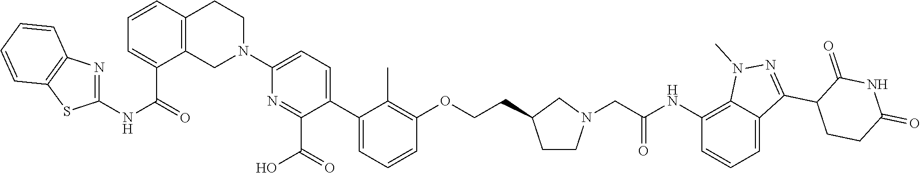 [SMILES: Cc1c(OCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R231b | 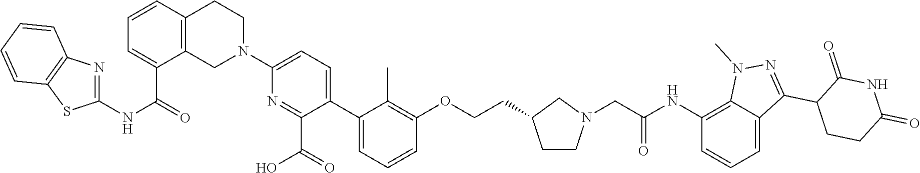 [SMILES: Cc1c(OCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R236 | 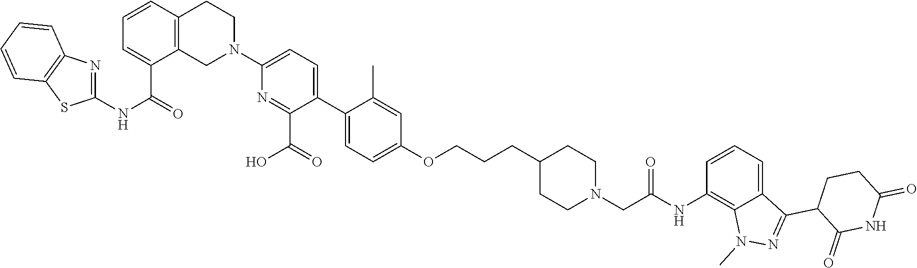 [SMILES: Cc1cc(OCCCC2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O] |
| R240 | 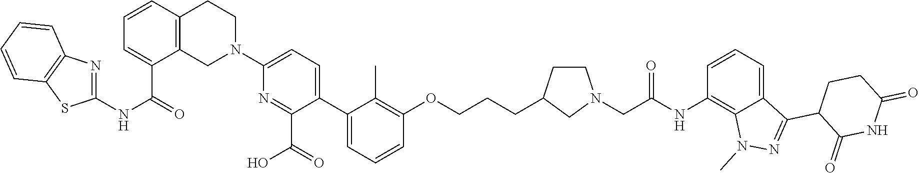 [SMILES: Cc1c(OCCCC2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R240a | 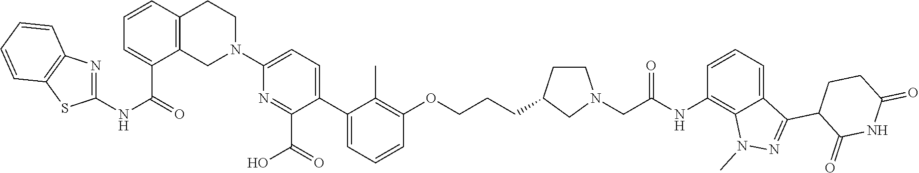 [SMILES: Cc1c(OCCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R240b | 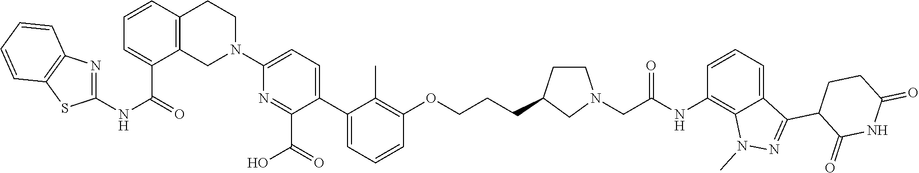 [SMILES: Cc1c(OCCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| R242 | 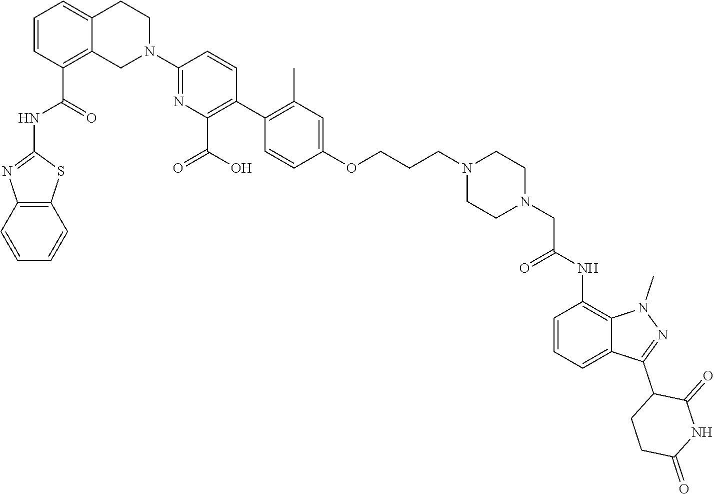 [SMILES: Cc1cc(OCCCN2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O] |
| R243 | 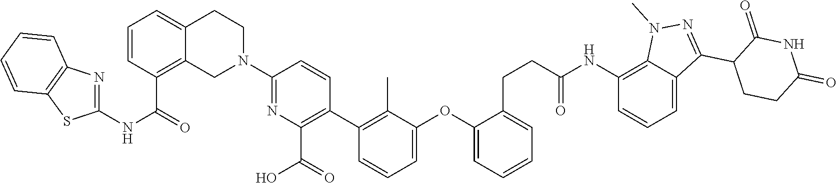 [SMILES: Cc1c(Oc2ccccc2CCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| R245 | 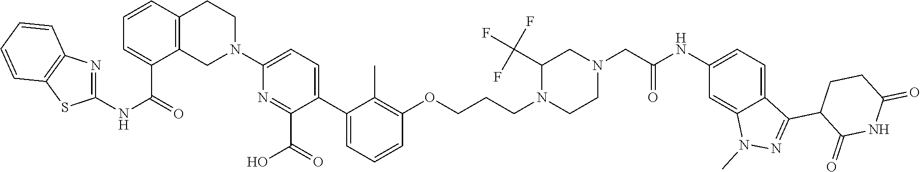 [SMILES: Cc1c(OCCCN2CCN(CC(=O)Nc3ccc4c(C5CCC(=O)NC5=O)nn(C)c4c3)CC2C(F)(F)F)cccc1-c1ccc(N2CCc3cccc(C(=O)Nc4nc5ccccc5s4)c3C2)nc1C(=O)O] |
| R245a | 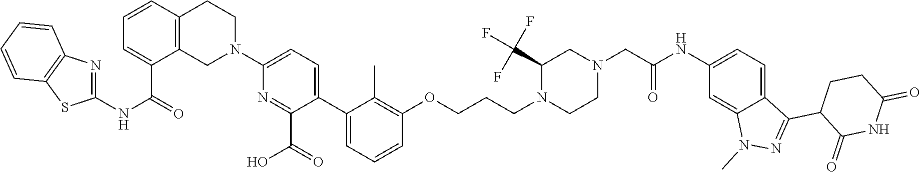 [SMILES: Cc1c(OCCCN2CCN(CC(=O)Nc3ccc4c(C5CCC(=O)NC5=O)nn(C)c4c3)C[C@@H]2C(F)(F)F)cccc1-c1ccc(N2CCc3cccc(C(=O)Nc4nc5ccccc5s4)c3C2)nc1C(=O)O] |
| R245b | 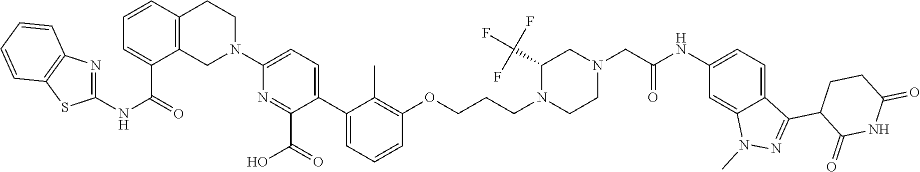 [SMILES: Cc1c(OCCCN2CCN(CC(=O)Nc3ccc4c(C5CCC(=O)NC5=O)nn(C)c4c3)C[C@H]2C(F)(F)F)cccc1-c1ccc(N2CCc3cccc(C(=O)Nc4nc5ccccc5s4)c3C2)nc1C(=O)O] |
| R246 | 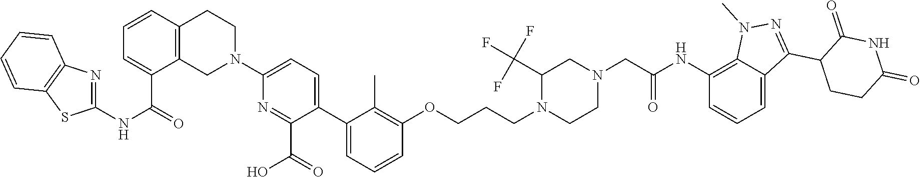 [SMILES: Cc1c(OCCCN2CCN(CC(=O)Nc3cccc4c(C5CCC(=O)NC5=O)nn(C)c34)CC2C(F)(F)F)cccc1-c1ccc(N2CCc3cccc(C(=O)Nc4nc5ccccc5s4)c3C2)nc1C(=O)O] |
| R246a | 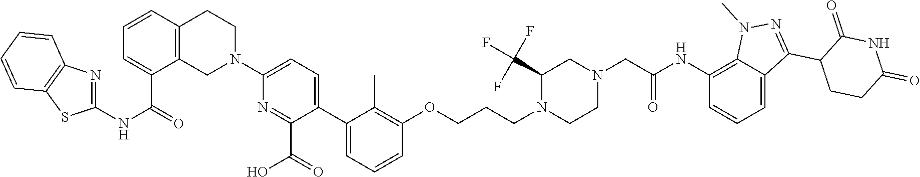 [SMILES: Cc1c(OCCCN2CCN(CC(=O)Nc3cccc4c(C5CCC(=O)NC5=O)nn(C)c34)C[C@@H]2C(F)(F)F)cccc1-c1ccc(N2CCc3cccc(C(=O)Nc4nc5ccccc5s4)c3C2)nc1C(=O)O] |
| R246b | 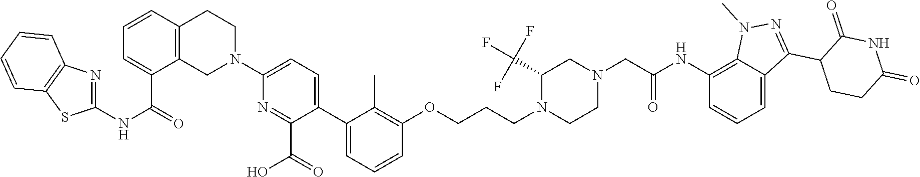 [SMILES: Cc1c(OCCCN2CCN(CC(=O)Nc3cccc4c(C5CCC(=O)NC5=O)nn(C)c34)C[C@H]2C(F)(F)F)cccc1-c1ccc(N2CCc3cccc(C(=O)Nc4nc5ccccc5s4)c3C2)nc1C(=O)O] |
| R262 | 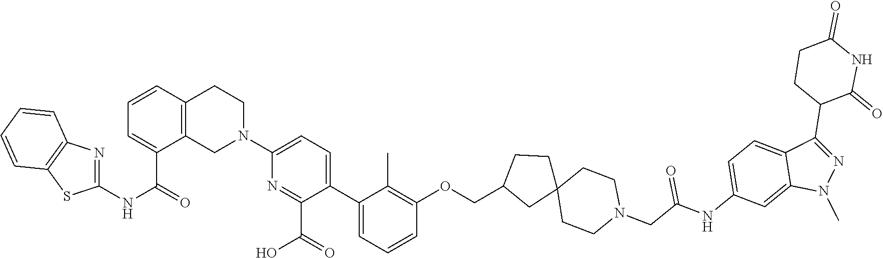 [SMILES: Cc1c(OCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R262a | 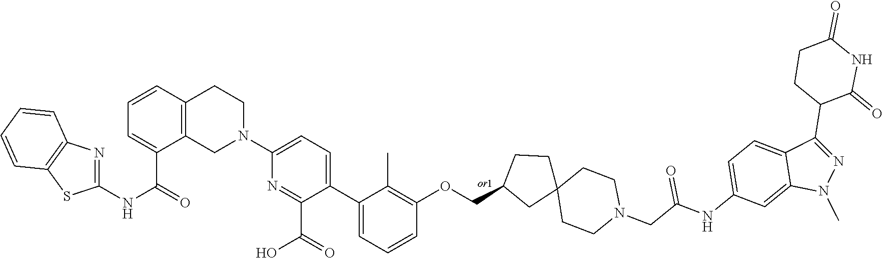 [SMILES: Cc1c(OC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R262b | 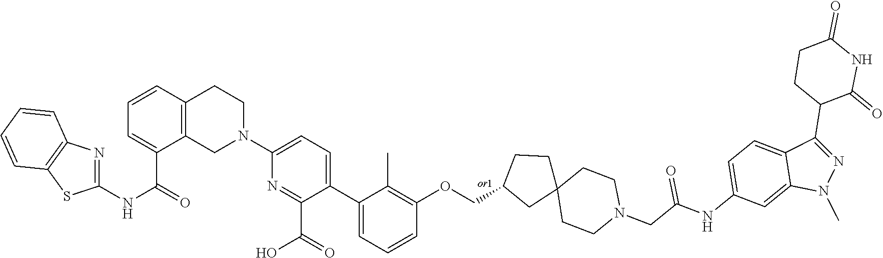 [SMILES: Cc1c(OC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R263 | 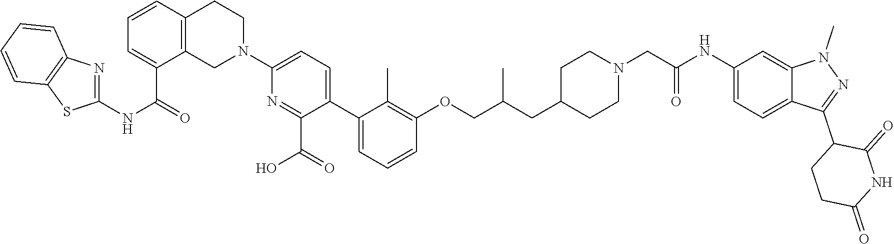 [SMILES: Cc1c(OCC(C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R263a | 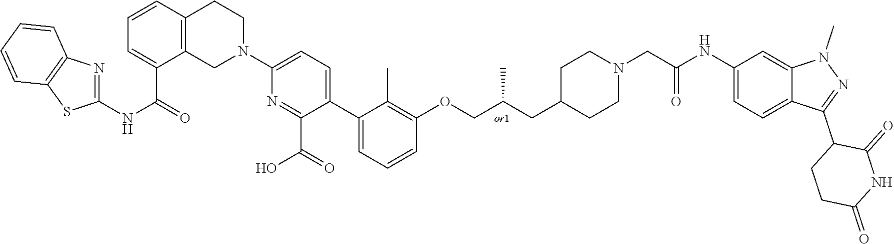 [SMILES: Cc1c(OC[C@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R263b | 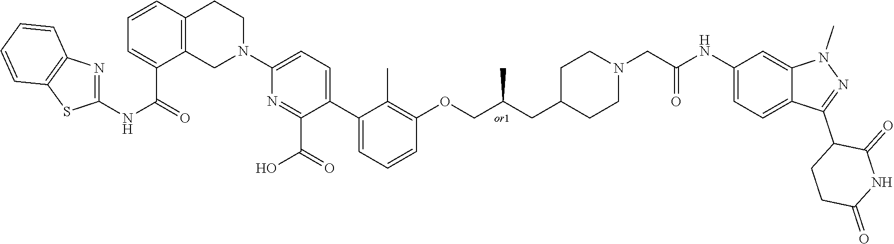 [SMILES: Cc1c(OC[C@@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R264 | 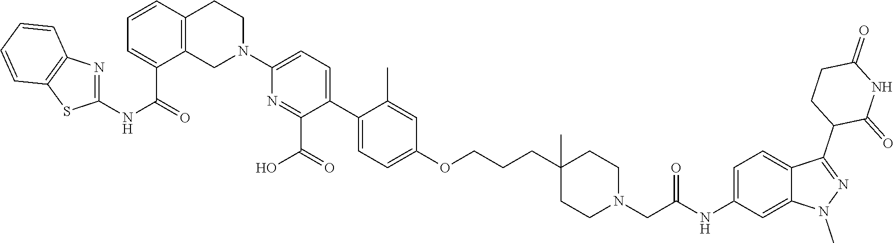 [SMILES: Cc1cc(OCCCC2(C)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R265 | 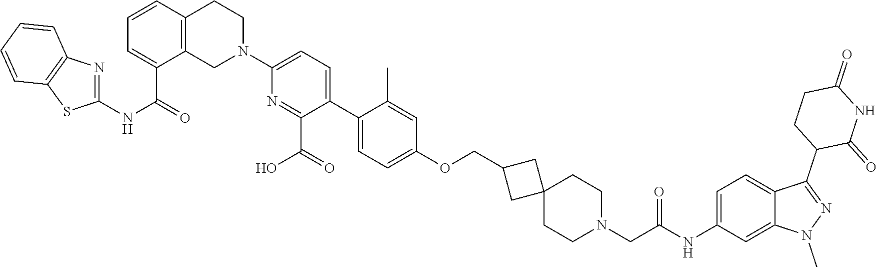 [SMILES: Cc1cc(OCC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O] |
| R266 | 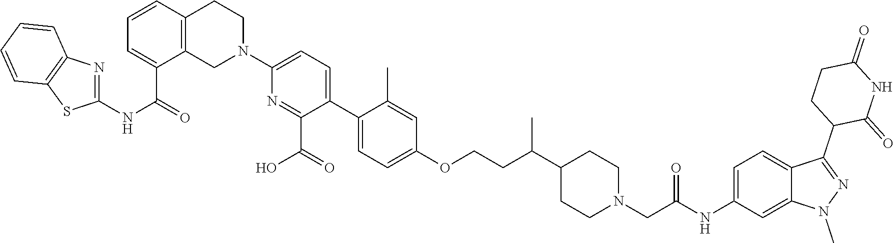 [SMILES: Cc1cc(OCCC(C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R266a | 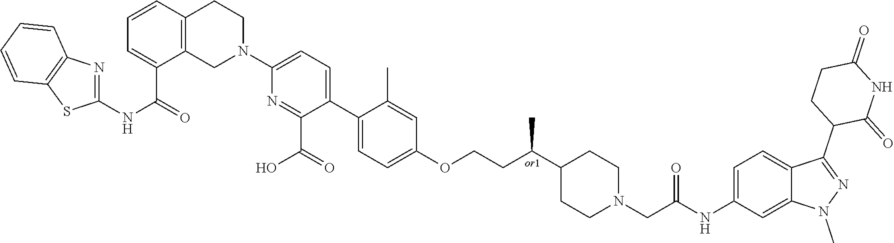 [SMILES: Cc1cc(OCC[C@@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R266b | 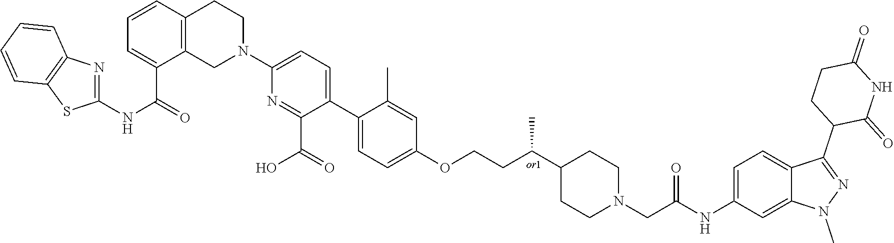 [SMILES: Cc1cc(OCC[C@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R267 | 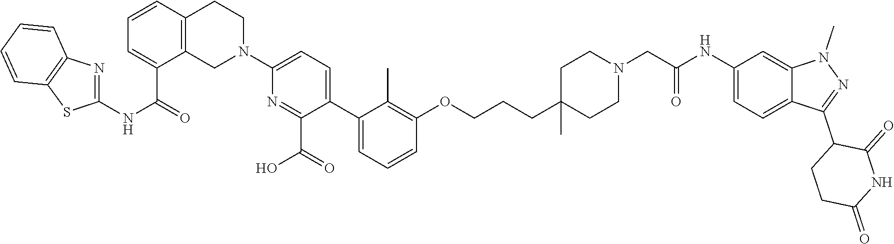 [SMILES: Cc1c(OCCCC2(C)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R268 | 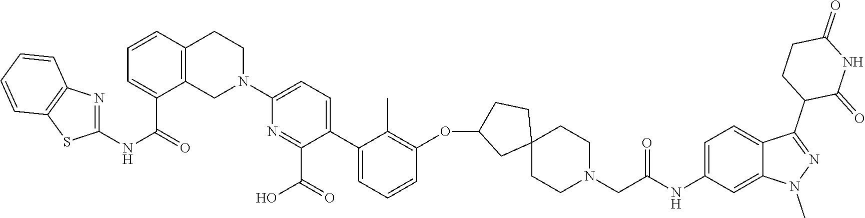 [SMILES: Cc1c(OC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R268a | 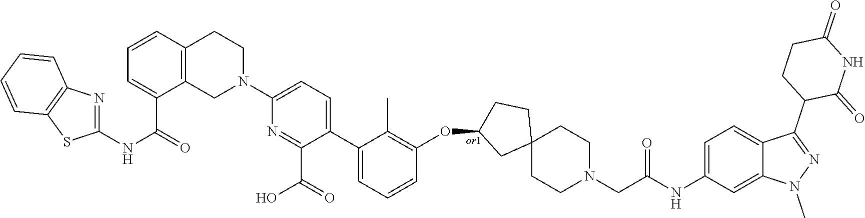 [SMILES: Cc1c(O[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R268b | 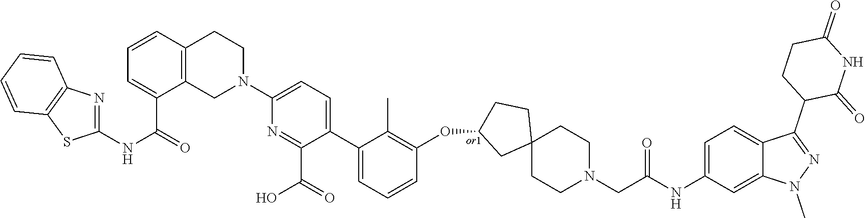 [SMILES: Cc1c(O[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R271 | 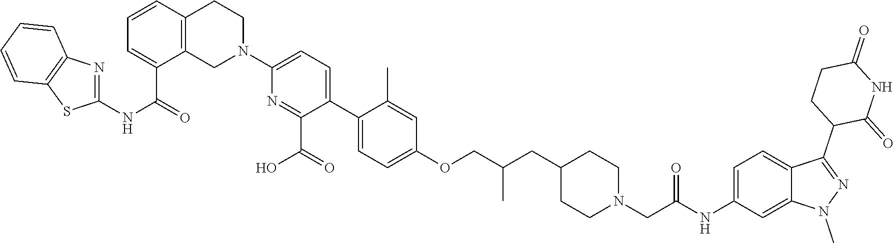 [SMILES: Cc1cc(OCC(C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R271a | 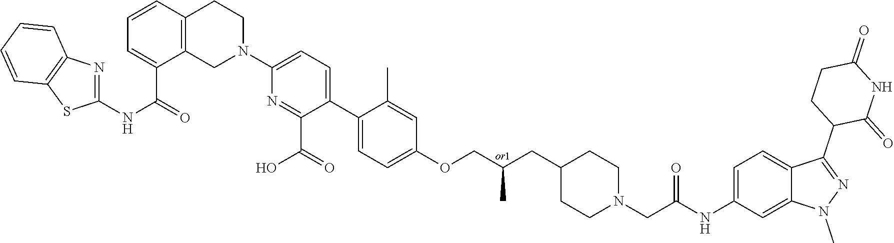 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R271b | 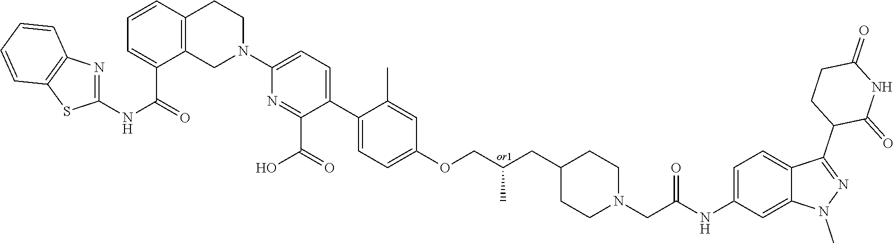 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R272 | 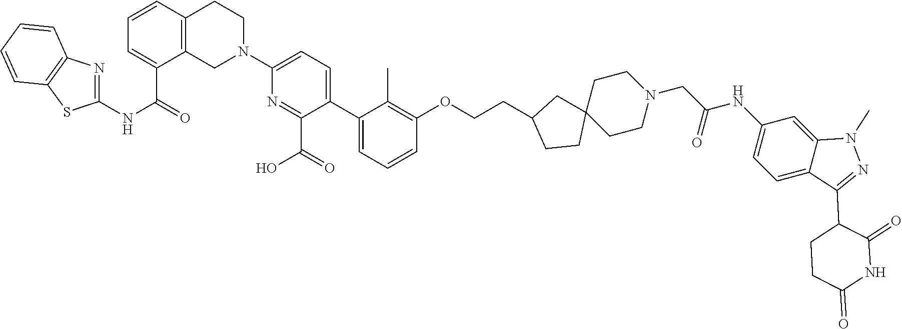 [SMILES: Cc1c(OCCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R272a | 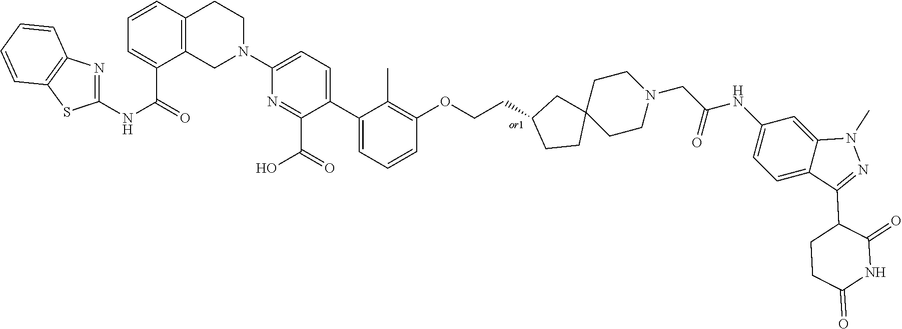 [SMILES: Cc1c(OCC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R272b | 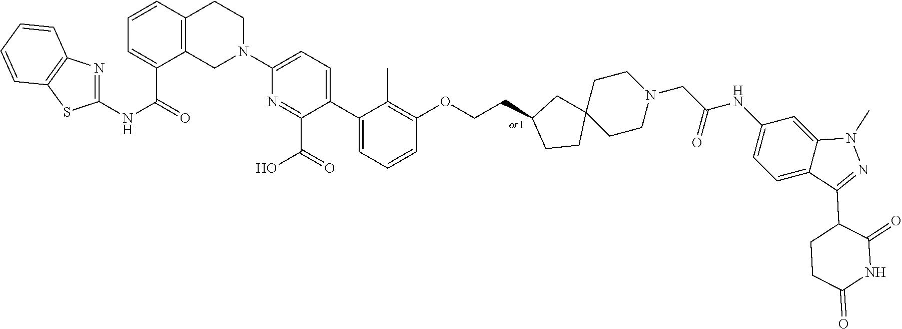 [SMILES: Cc1c(OCC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| R276 | 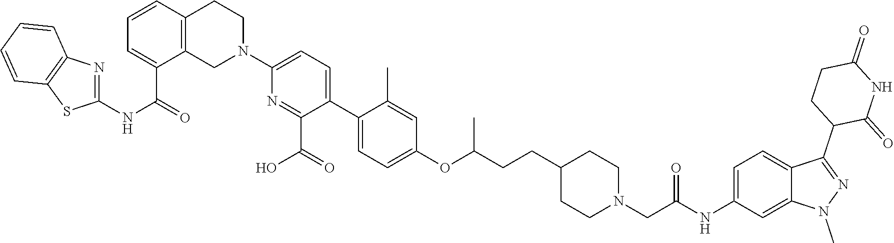 [SMILES: Cc1cc(OC(C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R276a | 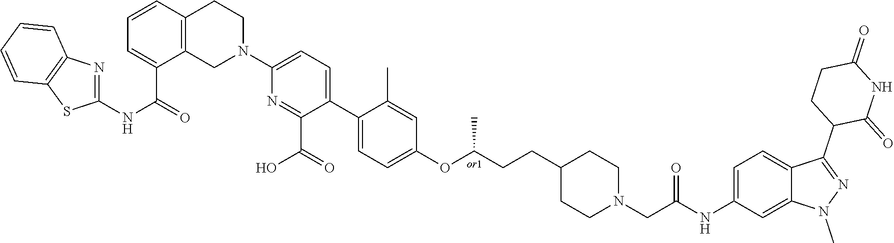 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R276b | 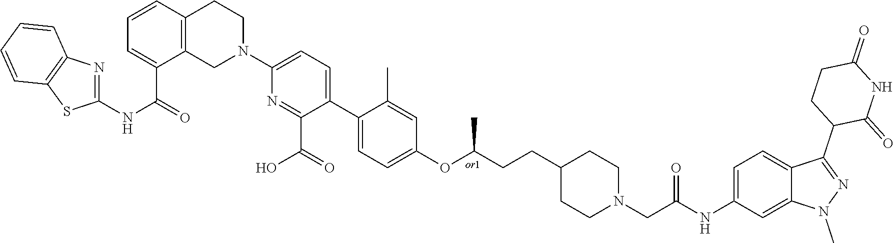 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R277 | 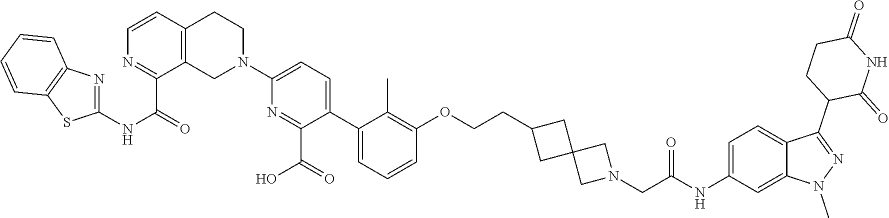 [SMILES: Cc1c(OCCC2CC3(C2)CN(CC(=O)Nc2ccc4c(C5CCC(=O)NC5=O)nn(C)c4c2)C3)cccc1-c1ccc(N2CCc3ccnc(C(=O)Nc4nc5ccccc5s4)c3C2)nc1C(=O)O] |
| R280 | 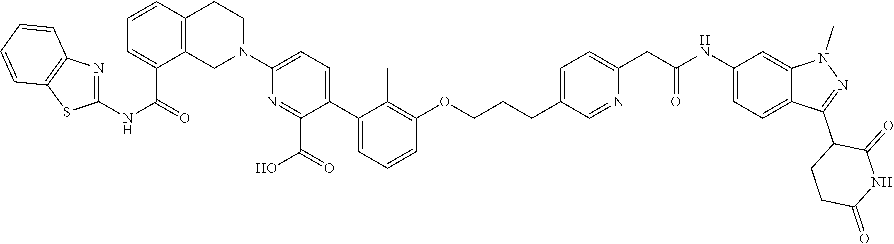 [SMILES: Cc1c(OCCCc2ccc(CC(=O)Nc3ccc4c(C5CCC(=O)NC5=O)nn(C)c4c3)nc2)cccc1-c1ccc(N2CCc3cccc(C(=O)Nc4nc5ccccc5s4)c3C2)nc1C(=O)O] |
| R286 | 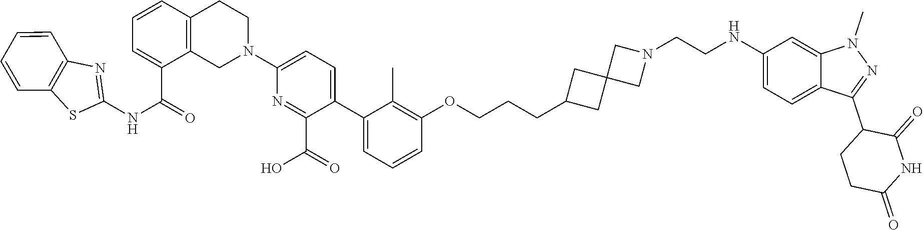 [SMILES: Cc1c(OCCCC2CC3(C2)CN(CCNc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O] |
| R289 | 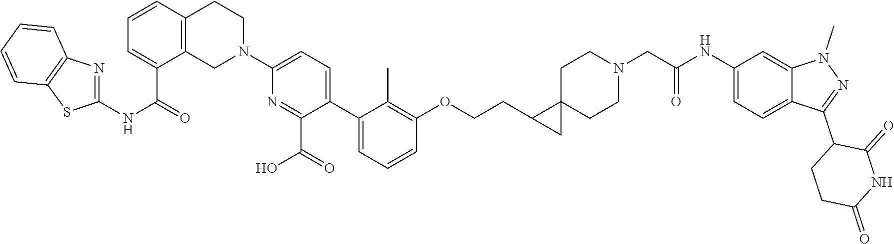 [SMILES: Cc1c(OCCC2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| R289a | 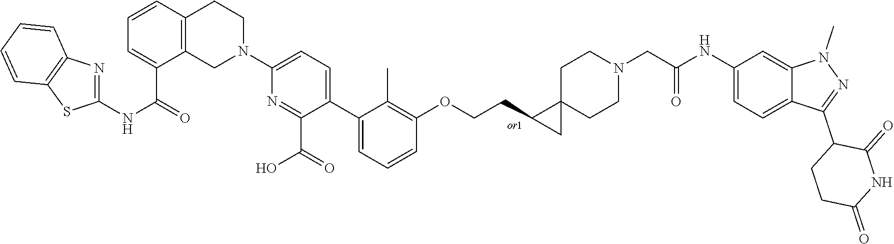 [SMILES: Cc1c(OCC[C@@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| R289b | 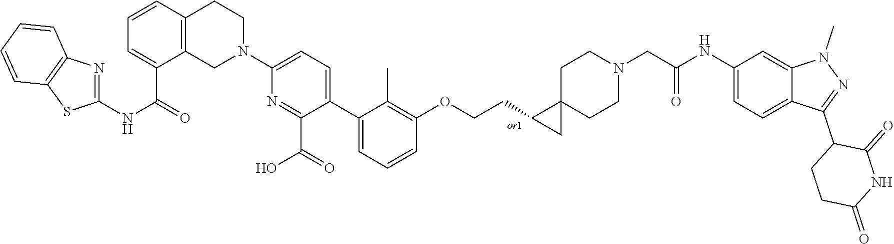 [SMILES: Cc1c(OCC[C@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| R290 | 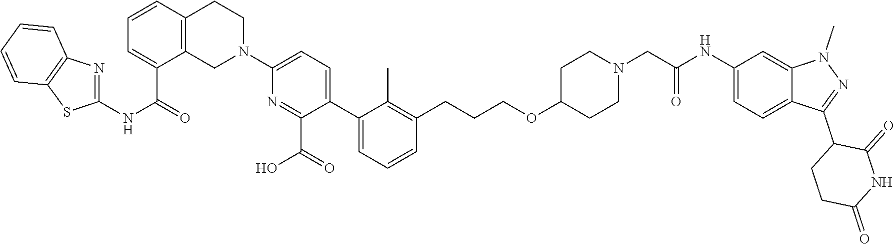 [SMILES: Cc1c(CCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R291 | 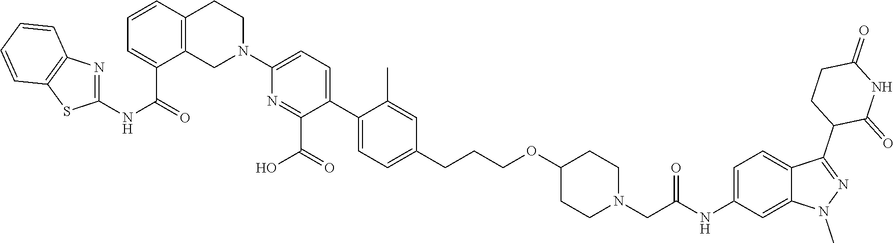 [SMILES: Cc1cc(CCCOC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R292 | 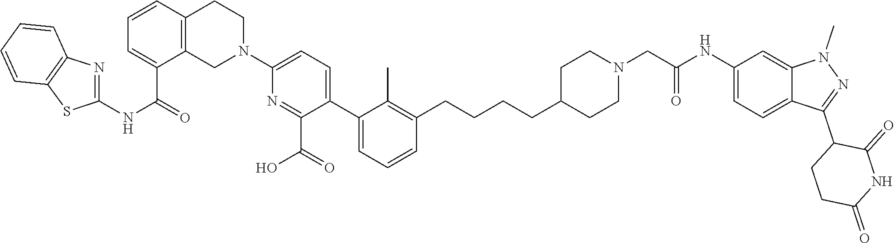 [SMILES: Cc1c(CCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R294 | 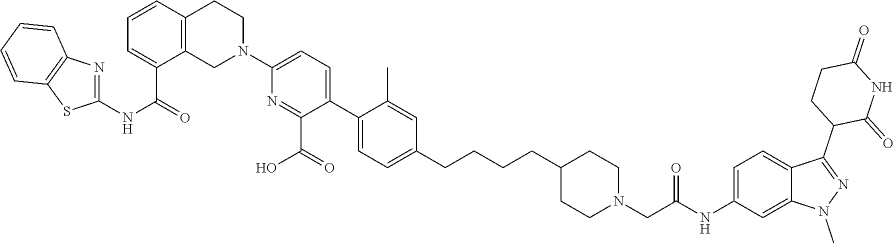 [SMILES: Cc1cc(CCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R295 | 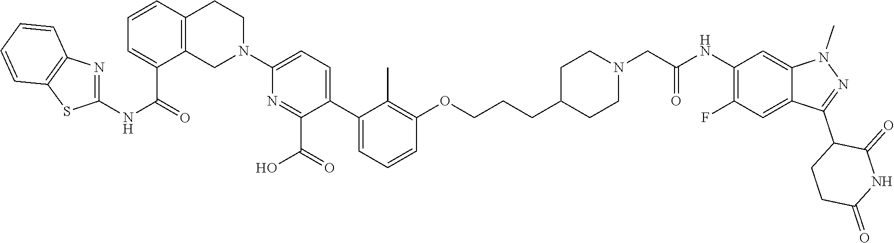 [SMILES: Cc1c(OCCCC2CCN(CC(=O)Nc3cc4c(cc3F)c(C3CCC(=O)NC3=O)nn4C)CC2)cccc1-c1ccc(N2CCc3cccc(C(=O)Nc4nc5ccccc5s4)c3C2)nc1C(=O)O] |
| R302 | 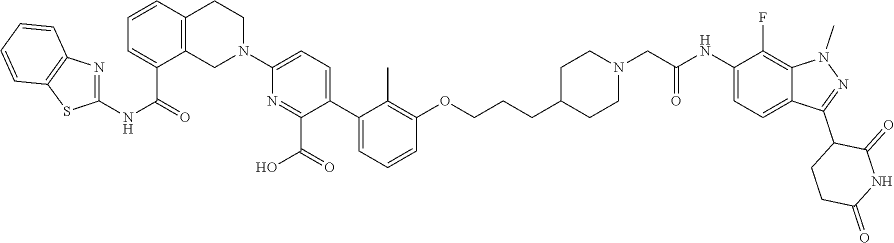 [SMILES: Cc1c(OCCCC2CCN(CC(=O)Nc3ccc4c(C5CCC(=O)NC5=O)nn(C)c4c3F)CC2)cccc1-c1ccc(N2CCc3cccc(C(=O)Nc4nc5ccccc5s4)c3C2)nc1C(=O)O] |
| R303 | 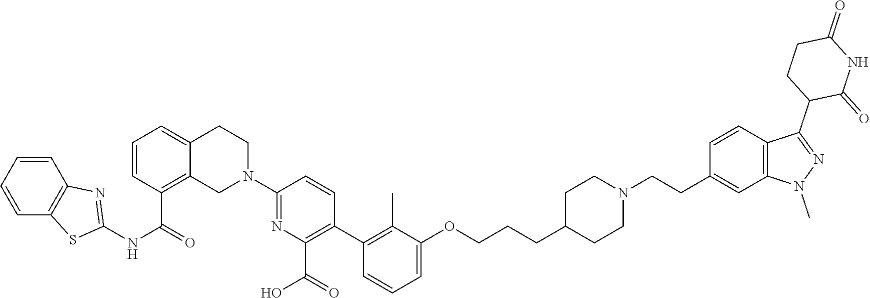 [SMILES: Cc1c(OCCCC2CCN(CCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R304 | 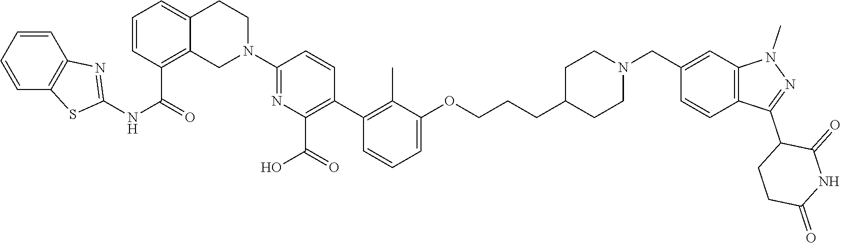 [SMILES: Cc1c(OCCCC2CCN(Cc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R305 | 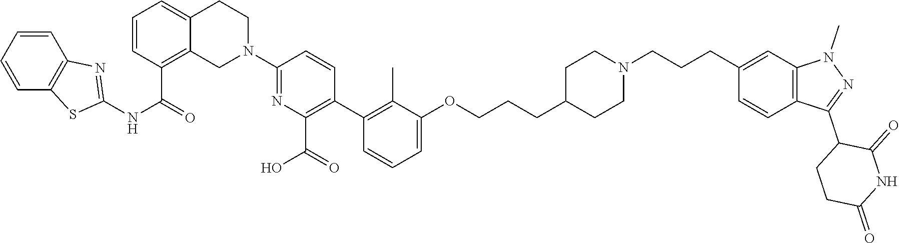 [SMILES: Cc1c(OCCCC2CCN(CCCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R309 | 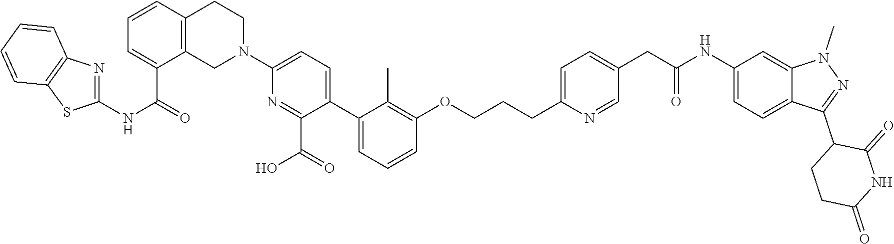 [SMILES: Cc1c(OCCCc2ccc(CC(=O)Nc3ccc4c(C5CCC(=O)NC5=O)nn(C)c4c3)cn2)cccc1-c1ccc(N2CCc3cccc(C(=O)Nc4nc5ccccc5s4)c3C2)nc1C(=O)O] |
| R313 | 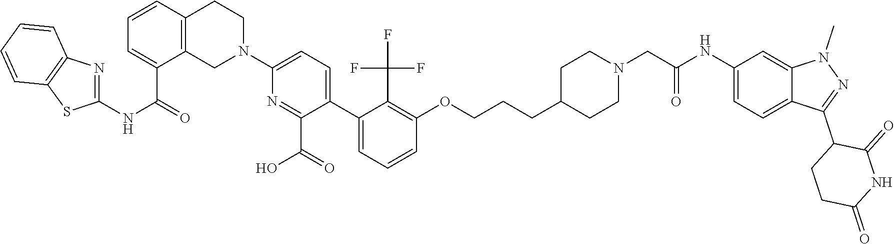 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)cc21] |
| R314 | 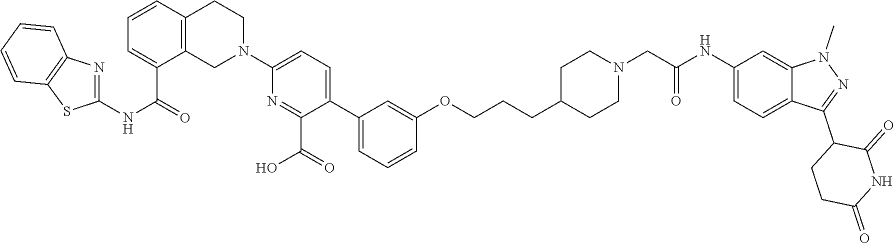 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4)CC3)cc21] |
| R317 | 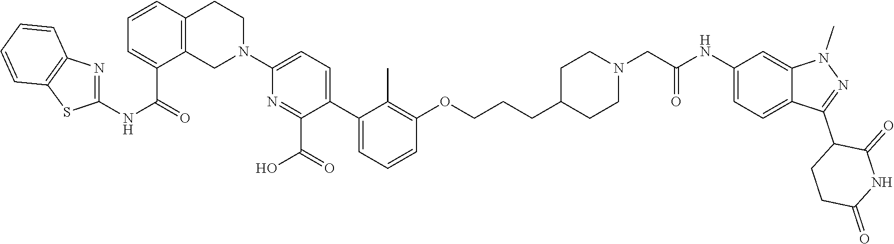 [SMILES: Cc1c(OCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| R343 | 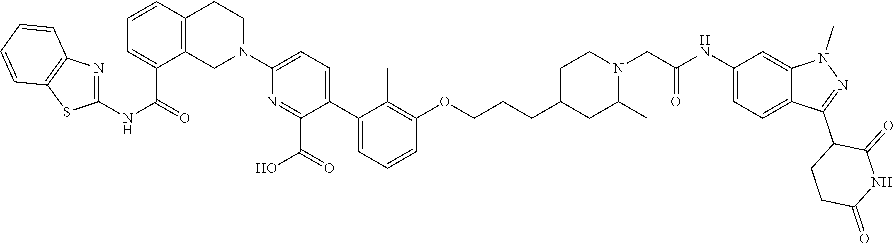 [SMILES: Cc1c(OCCCC2CCN(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| R343a | 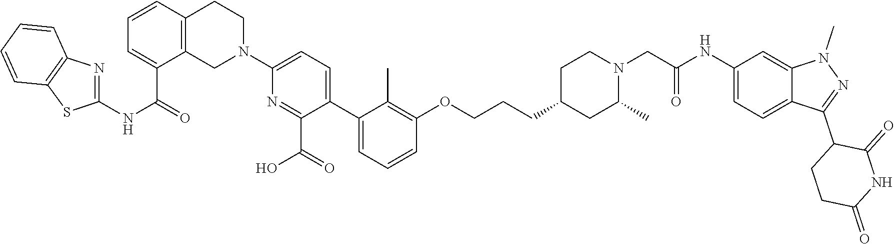 [SMILES: Cc1c(OCCC[C@@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O] |
| R343b | 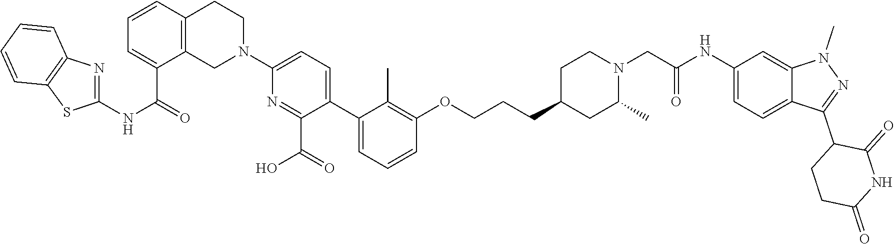 [SMILES: Cc1c(OCCC[C@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O] |
| R343c | 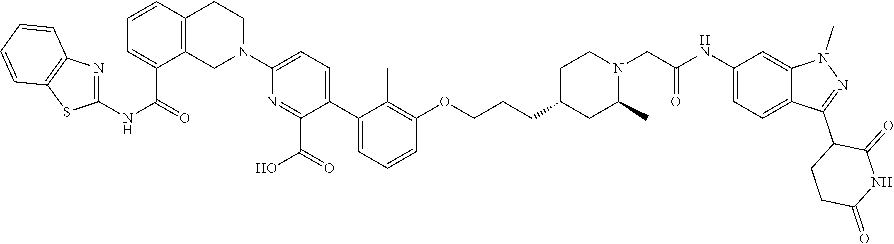 [SMILES: Cc1c(OCCC[C@@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| R343d | 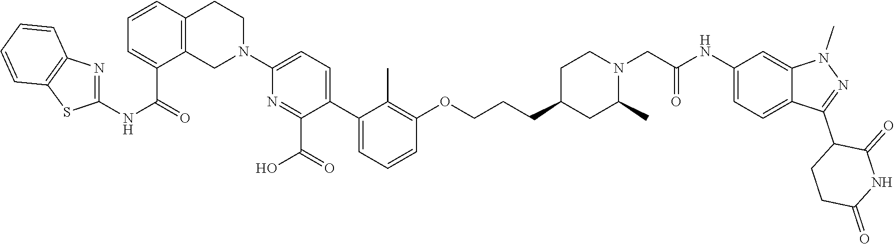 [SMILES: Cc1c(OCCC[C@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| R344 | 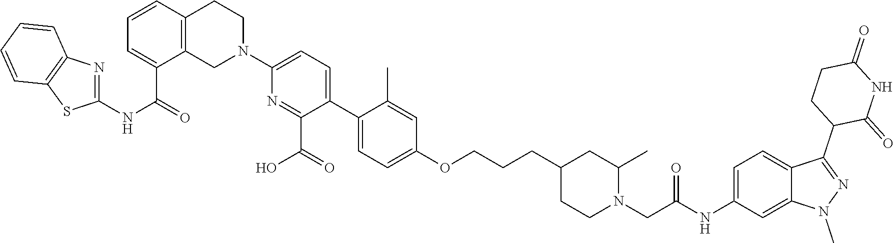 [SMILES: Cc1cc(OCCCC2CCN(CC(=O)Nc3ccc4c(C5CCC(=O)NC5=O)nn(C)c4c3)C(C)C2)ccc1-c1ccc(N2CCc3cccc(C(=O)Nc4nc5ccccc5s4)c3C2)nc1C(=O)O] |
| R344a | 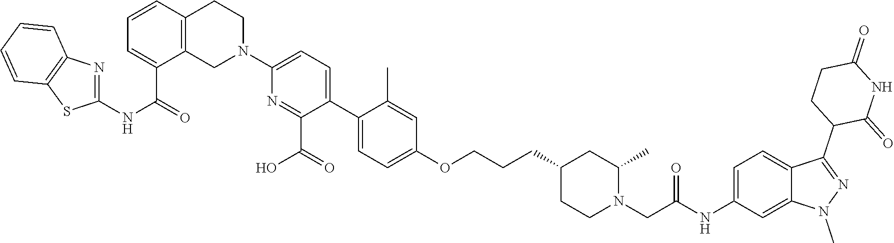 [SMILES: Cc1cc(OCCC[C@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O] |
| R344b | 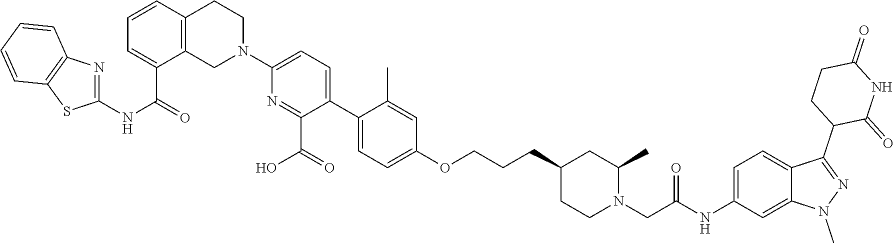 [SMILES: Cc1cc(OCCC[C@@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O] |
| R344c | 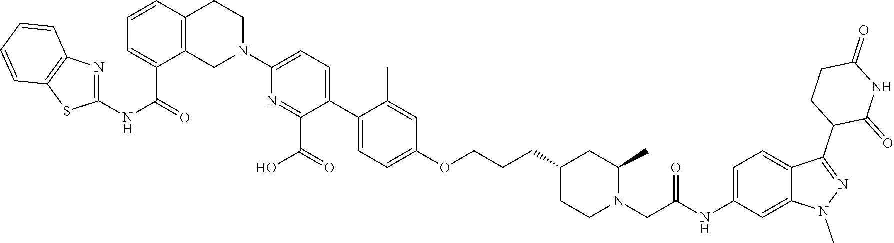 [SMILES: Cc1cc(OCCC[C@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O] |
| R344d | 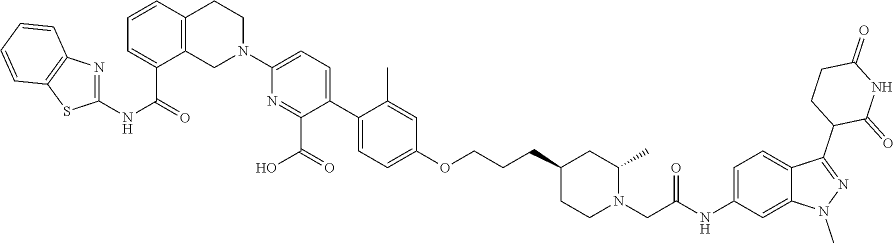 [SMILES: Cc1cc(OCCC[C@@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O] |
| R355 | 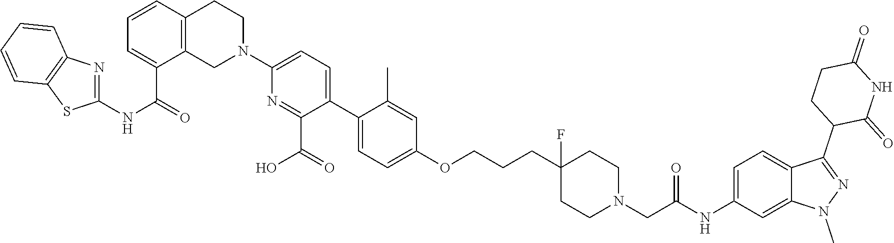 [SMILES: Cc1cc(OCCCC2(F)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| R356 | 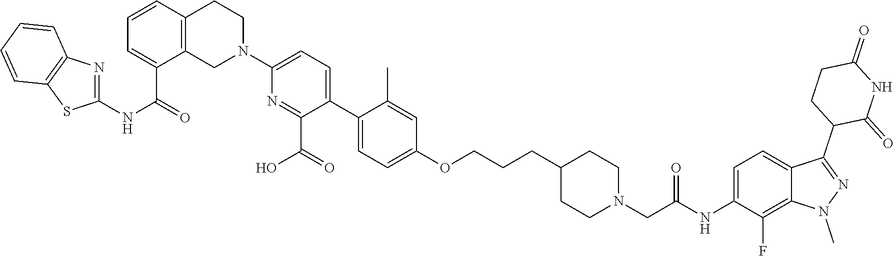 [SMILES: Cc1cc(OCCCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| R357 | 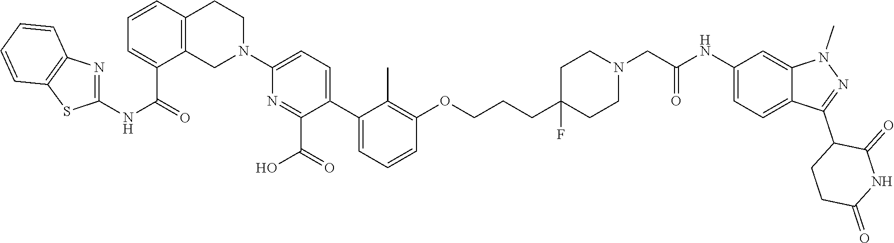 [SMILES: Cc1c(OCCCC2(F)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |

Also provided herein are compounds of Formula (II)

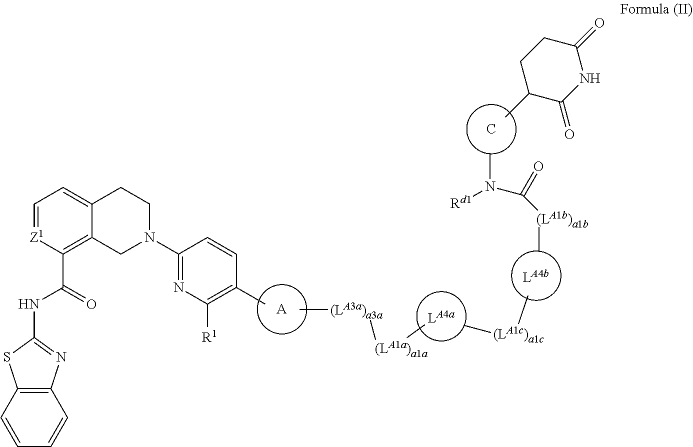

or pharmaceutically acceptable salts thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{g};
Ring A is selected from the group consisting of: (A1) and (A2):

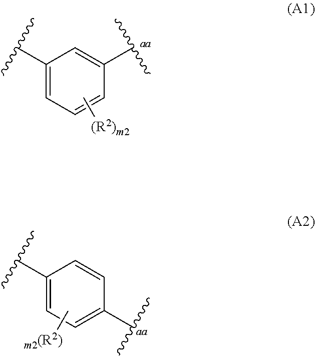

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to -(L^{A3a})ₐ₃ₐ-;
L^{A3a} is selected from the group consisting of: —N(R^{d})—, —O—, —S(O)₀₋₂—, and C(═O);
a3a is 0 or 1;
each L^{A1a} L^{A1b}, and L^{A1c} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
a1a, a1b, and a1c are each independently 0, 1, or 2;
L^{A}4a and L^{A4b} are independently selected from the group consisting of: C₃₋₈ cycloalkylene and 4-12 membered heterocyclylene, each of which is optionally substituted with 1-3 R^{L4};
wherein each R^{L4} is independently selected from the group consisting of: —F, CN, OH, C₁₋₃ alkoxy, and C₁₋₃ alkyl optionally substituted with 1-3 R^{c};
R^{d1} is —H or C₁₋₃ alkyl;
Ring C is selected from the group consisting of:

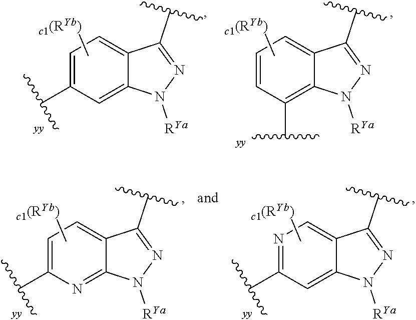

wherein:
R^{Ya} is selected from the group consisting of: H and C₁₋₆ alkyl optionally substituted with 1-3 R^{c}
each R^{Yb} is independently selected from the group consisting of: F, C₁₋₃ alkoxy, or C1-3 alkyl optionally substituted with 1-3 R^{c};
c1 is 0, 1, or 2; and
yy represents the point of attachment to —N(R^{d1})—;
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂ (C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C1-3 haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (II), Z¹ is CH.

In some embodiments of Formula (II), Ring A is

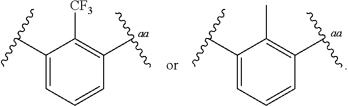

In some embodiments of Formula (II), Ring A is

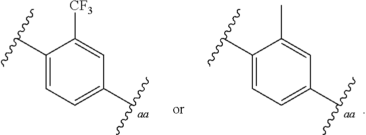

In some embodiments of Formula (II), a3a is 1.

In some embodiments of Formula (II), L^{A3a} is —O—.

In some embodiments of Formula (II), a1a is 1.

In some embodiments of Formula (II), L^{A1a} is —CH₂—.

In some embodiments of Formula (II), a1c is 0 or 1.

In some embodiments of Formula (II), L^{A1c} is —CH₂—.

In some embodiments of Formula (II), a1b is 1.

In some embodiments of Formula (II), L^{A1b} is —CH₂—.

In some embodiments of Formula (II), R^{d1} is —H.

In some embodiments of Formula (II), L^{A4b} is a 5-12 membered nitrogen-containing heterocyclylene optionally substituted with 1-3 R^{L4}. In some embodiments, L^{A4b} is a monocyclic 5-6 membered nitrogen-containing heterocyclylene optionally substituted with 1-3 R^{L4}.

In some embodiments of Formula (II), L^{A4a} is a C₃₋₆ cycloalkylene optionally substituted with 1-2 R^{L4}.

In some embodiments of Formula (II), each R^{L4} is independently selected from the group consisting of: —F, CN, OH, C₁₋₃ alkoxy, and C₁₋₃ alkyl optionally substituted with 1-3 F.

In some embodiments of Formula (II), L^{A4b} is

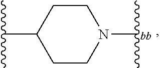

wherein bb represents point of attachment to -(L^{A1b})_{a1b}-.

In some embodiments of Formula (II), L^{A4a} is

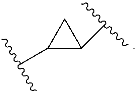 [SMILES: *C1CC1*]

In some embodiments of Formula (II), Ring C is

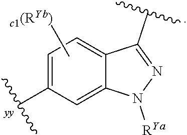

In some embodiments of Formula (II), c1 is 0.

In some embodiments of Formula (II), R^{Ya} is C₁₋₆ alkyl optionally substituted with 1-3 —F. For example, R^{Ya} can be methyl.

In some embodiments of Formula (II), R¹ is C(O)OH.

In some embodiments, the compounds of Formula (II) are compounds of Formula (II-a1) or (II-a2):

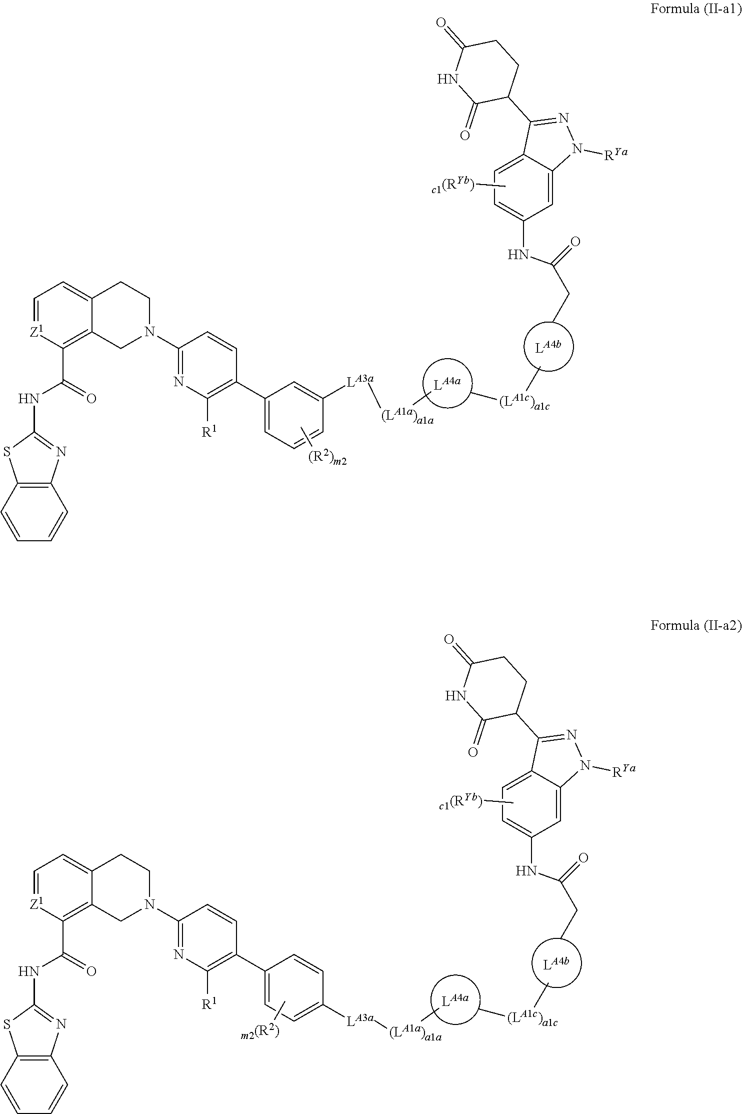

or pharmaceutically acceptable salts thereof, wherein:
Z¹ is CH or N;
L^{A3a} is selected from the group consisting of: —N(R^{d})— and —O—;
each L^{A1a} and L^{A1c} is independently selected from the group consisting of: —CH₂—, —CHR^{L}—, and —C(R^{L})₂—, wherein:
each R^{L} is independently selected from the group consisting of: halo and C₁₋₃ alkyl optionally substituted with 1-3 —F;
a1a and a1c are each independently 0, 1, or 2;
L^{A4a} is a C₃₋₆ cycloalkylene optionally substituted with 1-2 R^{L4};
L^{A4b} is a monocyclic 5-6 membered nitrogen-containing heterocyclylene optionally substituted with 1-3 R^{L4}; and
each R^{L4} is independently selected from the group consisting of: —F, CN, OH, C₁₋₃ alkoxy, and C₁₋₃ alkyl optionally substituted with 1-3 F.

In some embodiments of Formula (II-a1) or (II-a2), a1a is 1; and L^{A1a} is —CH₂—.

In some embodiments of Formula (II-a1) or (II-a2), a1c is 0 or 1.

In some embodiments of Formula (II-a1) or (II-a2), L^{A1c} is —CH₂—.

In some embodiments of Formula (II-a1) or (II-a2), L^{A4a} is

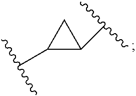 [SMILES: *C1CC1*]

### and L^{A4b} is

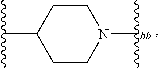

wherein bb represents point of attachment to

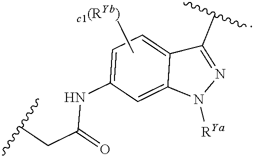

In some embodiments of Formula (II-a1) or (II-a2), c1 is 0; and R^{Ya} is C₁₋₃ alkyl (e.g., methyl).

In some embodiments of Formula (II-a1) or (II-a2), m2 is 1; and R² is CH₃ or CF₃.

In some embodiments of Formula (II-a1) or (II-a2), Z¹ is CH.

In some embodiments of Formula (II-a1) or (II-a2), R¹ is C(O)OH.

In some embodiments, the compounds of Formula (II) (e.g., Formula (II-a1) or (II-a2)) are selected from the group consisting of the compounds depicted in Table C2, or pharmaceutically acceptable salts thereof.

TABLE C2

| No. | Compound Structure |
|---|---|
| 1001 | 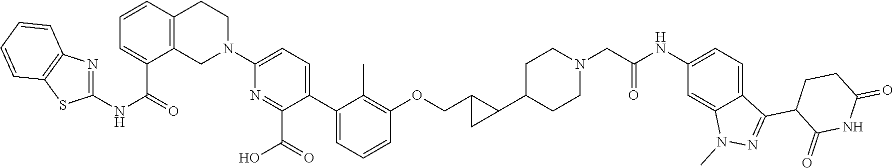 [SMILES: Cc1c(OCC2CC2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1001a | 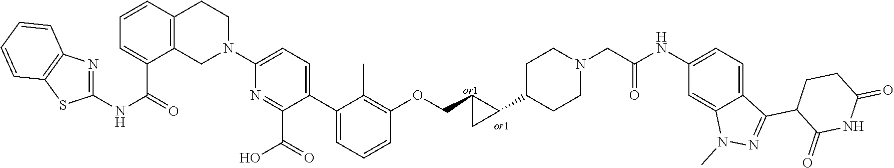 [SMILES: Cc1c(OC[C@@H]2C[C@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1001b | 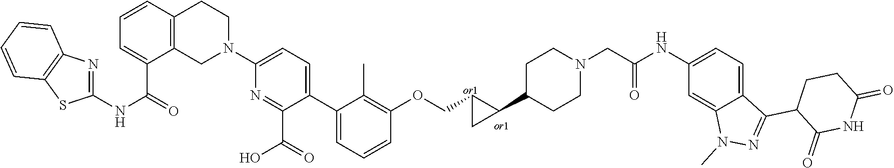 [SMILES: Cc1c(OC[C@H]2C[C@@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1001c | 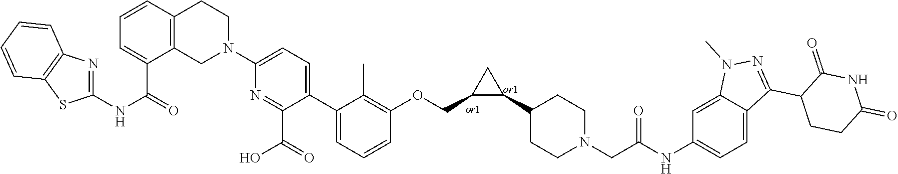 [SMILES: Cc1c(OC[C@H]2C[C@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1001d | 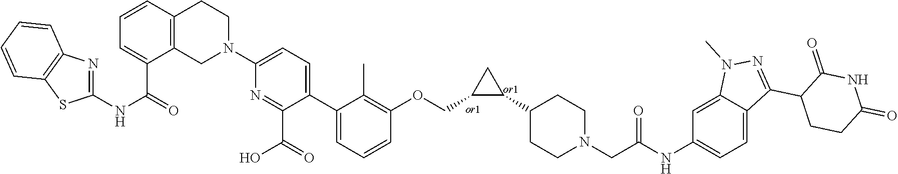 [SMILES: Cc1c(OC[C@@H]2C[C@@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1002 | 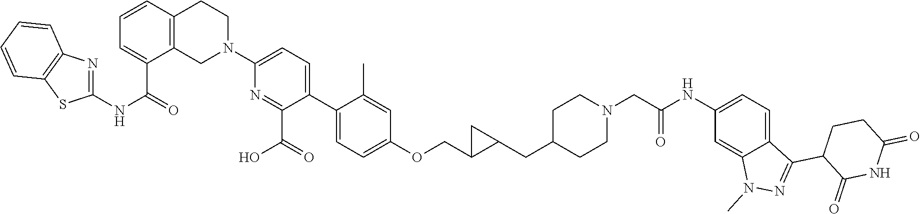 [SMILES: Cc1cc(OCC2CC2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1002a | 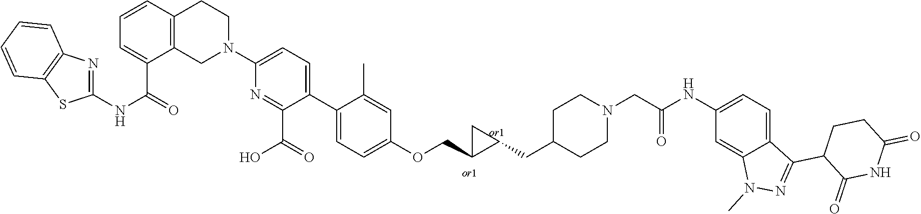 [SMILES: Cc1cc(OC[C@H]2C[C@@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1002b | 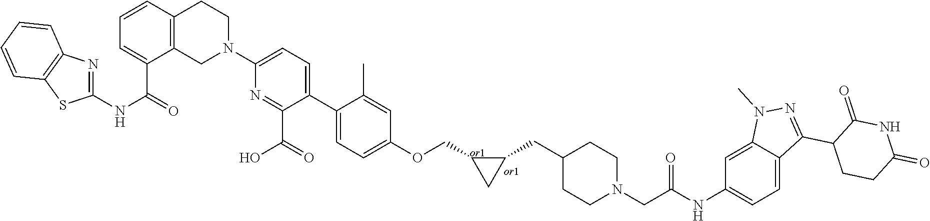 [SMILES: Cc1cc(OC[C@H]2C[C@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1002c | 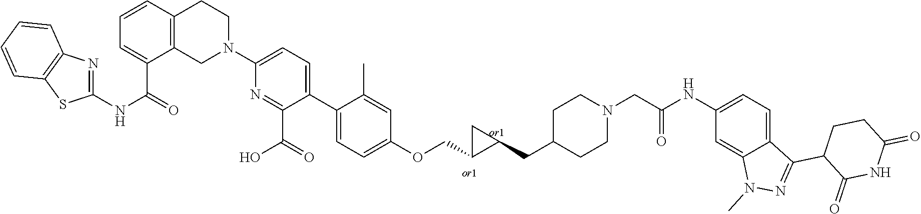 [SMILES: Cc1cc(OC[C@@H]2C[C@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1002d | 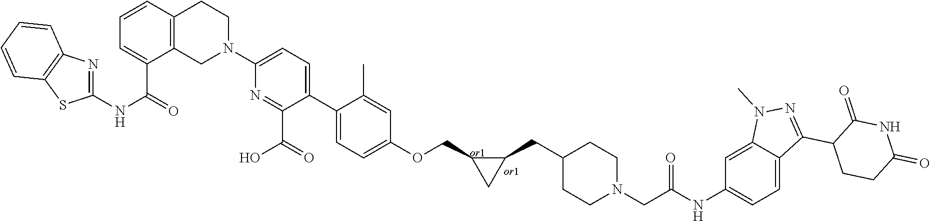 [SMILES: Cc1cc(OC[C@@H]2C[C@@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003 | 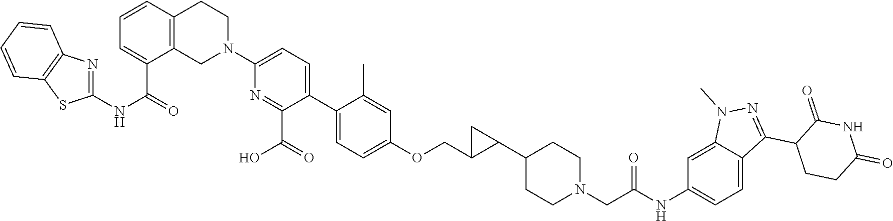 [SMILES: Cc1cc(OCC2CC2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003a | 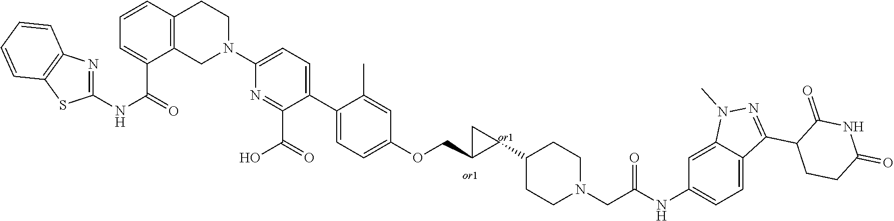 [SMILES: Cc1cc(OC[C@H]2C[C@@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003b | 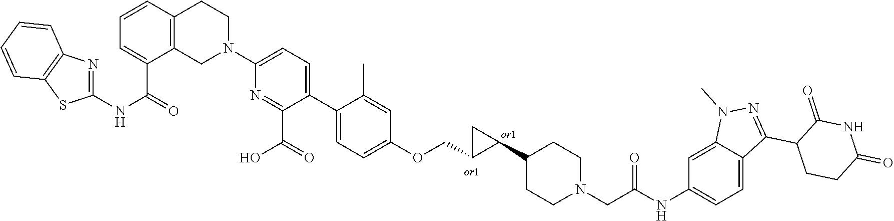 [SMILES: Cc1cc(OC[C@@H]2C[C@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003c | 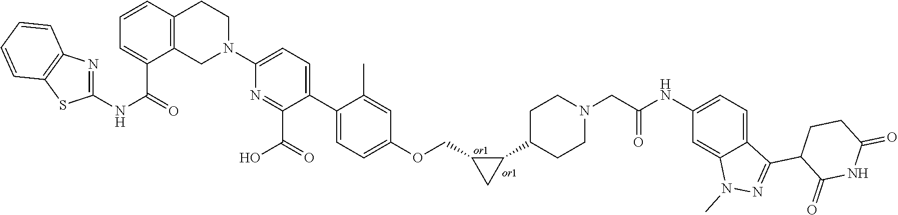 [SMILES: Cc1cc(OC[C@H]2C[C@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003d | 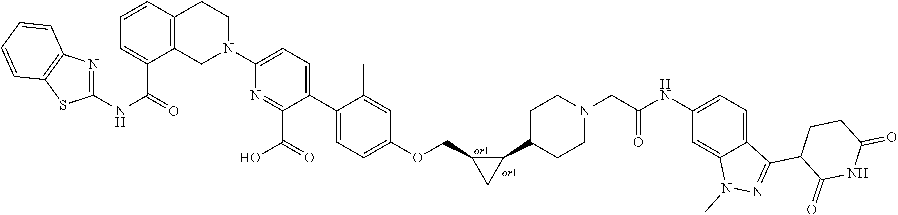 [SMILES: Cc1cc(OC[C@@H]2C[C@@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1004 | 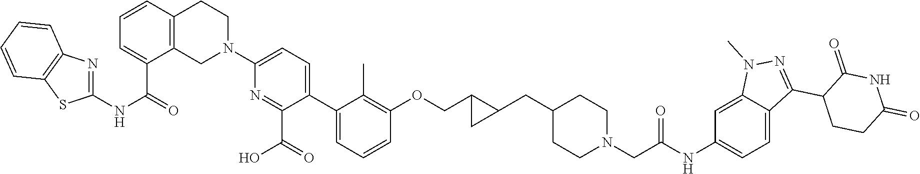 [SMILES: Cc1c(OCC2CC2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1004a | 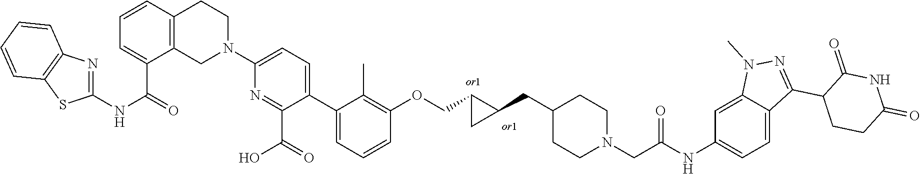 [SMILES: Cc1c(OC[C@H]2C[C@@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1004b | 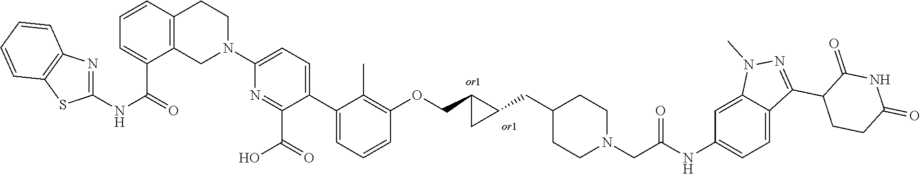 [SMILES: Cc1c(OC[C@@H]2C[C@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1004c | 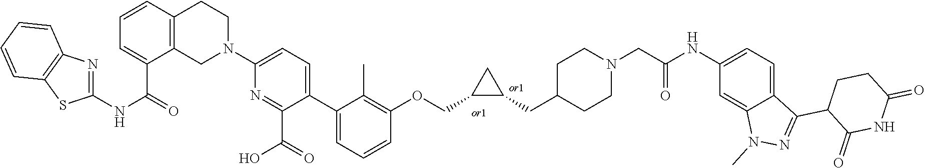 [SMILES: Cc1c(OC[C@@H]2C[C@@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1004d | 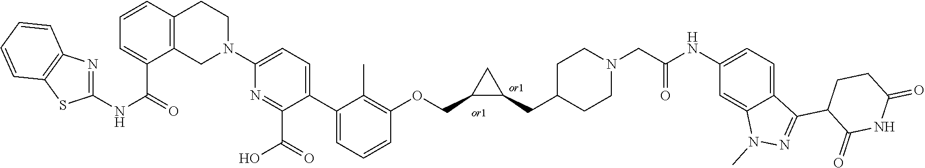 [SMILES: Cc1c(OC[C@H]2C[C@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |

Also provided herein are compounds of Formula (A):

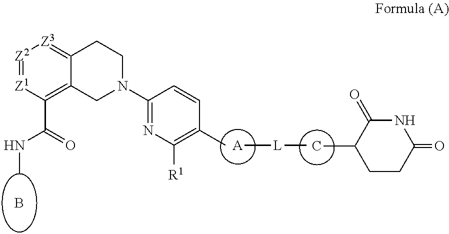

or pharmaceutically acceptable salts thereof, wherein:
Z¹, Z², and Z³ are independently selected from the group consisting of: CH, CR⁴, and N (e.g., CH);
R¹ is selected from the group consisting of:
(a) —C(O) OH;
(b) —C(O) OC₁₋₆ alkyl, wherein the C₁₋₆ alkyl is optionally substituted with 1-3 R^{c};
(c) —C(O)—(C₀₋₃ alkylene)-phenyl, wherein the phenyl is optionally substituted with 1-3 R^{a};
(d) —P(═O)(OH)₂; and
(e) —P(═O)(OC₁₋₆ alkyl)₂, wherein each of the C₁₋₆ alkyl is optionally substituted with 1-3 R^{c};
Ring A is selected from the group consisting of: C₄₋₁₀ cycloalkylene, C₆₋₁₀ arylene, and 5-10 membered heteroarylene, each of which is optionally substituted with 1-2 R²;
Ring B is selected from the group consisting of: C₆₋₁₀ aryl and 5-10 membered heteroaryl, each of which is optionally substituted with 1-3 R³;
each R², R³, and R⁴ is independently selected from the group consisting of: halo, —CN, C₁₋₃ alkoxy, C₁₋₃ haloalkoxy, and C₁₋₃ alkyl optionally substituted with 1-3 R^{c};
L is -(L^{A})ₙ₁-, wherein:
n1 is an integer from 4 to 10,
one L^{A} is L^{A4},
0-3 L^{A} are independently selected L^{A3}, and
each remaining L^{A} is an independently selected L^{A1};
each L^{A1} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
each L^{A3} is independently selected from the group consisting of: —N(R^{d})—, —N(R^{h})—, —O—, —S(O)₀₋₂—, and C(═O);
L^{A4} is independently selected from the group consisting of:
(a) C₃₋₁₂ cycloalkylene or 4-15 membered heterocyclylene, each of which is optionally substituted with 1-3 R^{L4}; and
(b) phenylene or 5-6 membered heteroarylene, each of which is optionally substituted with 1-3 R^{L4},
wherein each R^{L4} is independently selected from the group consisting of R^{a} and R^{b};
Ring C is selected from the group consisting of: phenylene and 5-15 membered heteroarylene, each of which is optionally substituted with 1-4 R^{Yb},
each R^{Yb} is independently selected from the group consisting of: R^{a} and R^{b};
each R^{a} is independently selected from the group consisting of:
(a) halo;
(b) —CN;
(c) —OH;
(d) oxo;
(e) C₁₋₆ alkoxy optionally substituted with 1-6 R^{c};
(f) —NR^{d}R^{e};
(g) —C(═O)C₁₋₆ alkyl;
(h) —C(═O)OC₁₋₆ alkyl;
(i) —C(═O)N(R^{f})₂;
(j) —S(O)₀₋₂(C₁₋₆ alkyl);
(k) —S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (A), Z¹ is CH or N.

In some embodiments of Formula (A), Z² is CH.

In some embodiments of Formula (A), Z³ is CH.

In some embodiments of Formula (A), Ring B is

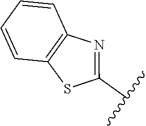 [SMILES: *c1nc2ccccc2s1]

optionally substituted with 1-2 R³ (e.g., Ring B is

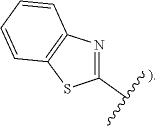 [SMILES: *c1nc2ccccc2s1]

In some embodiments of Formula (A), Ring B is phenyl optionally substituted with 1-2 R³ (e.g., Ring B is phenyl).

In some embodiments of Formula (A), Ring B is a 5-6 membered heteroaryl optionally substituted with 1-2 R³.

In some embodiments of Formula (A), Ring A is selected from the group consisting of: C₆₋₁₀ cycloalkylene (e.g., cyclohexylene), phenylene, and 5-6 membered heteroarylene, each of which is optionally substituted with 1-2 R². In some embodiments of Formula (A), Ring A is phenylene optionally substituted with 1-2 R². In some embodiments of Formula (A), Ring A is as defined for Formula (I) herein.

In some embodiments of Formula (A), R¹ is —C(O)OH.

In some embodiments of Formula (A), R¹ is —C(O)O^{t}Bu.

In some embodiments of Formula (A), R¹ is —P(═O)(OH)₂.

In some embodiments of Formula (A), Ring C is as defined for Formula (I) herein.

In some embodiments of Formula (A), Ring C is a 9-15 (e.g., 9-) membered heteroarylene optionally substituted with 1-3 R^{Yb}. In some embodiments, each R^{Yb} is independently selected from the group consisting of: —F, C₁₋₃ alkoxy, and C₁₋₃ alkyl optionally substituted with 1-3 R^{c}.

In some embodiments of Formula (A), Ring C is selected from the group consisting of:

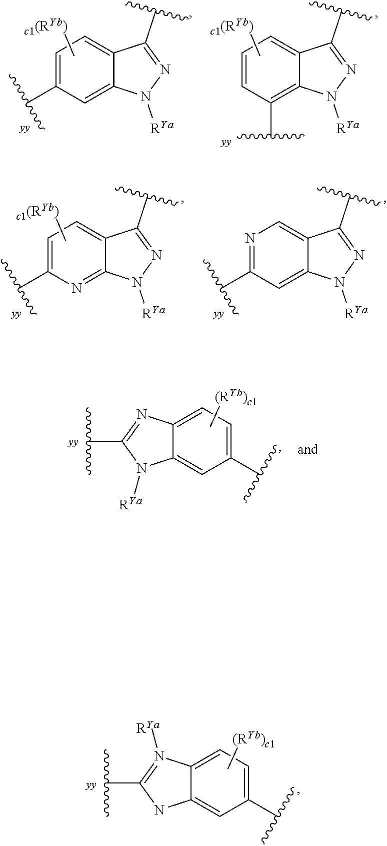

wherein yy represents the point of attachment to L; R^{Ya} is —H or C₁₋₃ alkyl optionally substituted with 1-3 R^{c}; c1 is 0, 1, or 2; and each R^{Yb} is independently selected from the group consisting of: —F, C₁₋₃ alkoxy, and C₁₋₃ alkyl optionally substituted with 1-3 —F (e.g., each R^{Yb} is independently selected from the group consisting of: —F, —OMe, —CH₃, —CHF₂, —CH₂F, and —CF₃). In some embodiments, R^{Ya} is —H or methyl.

In some embodiments of Formula (A), L is as defined for Formula (I) herein.

In some embodiments of Formula (A), n1 is an integer from 5 to 9; and L is selected from the group consisting of:

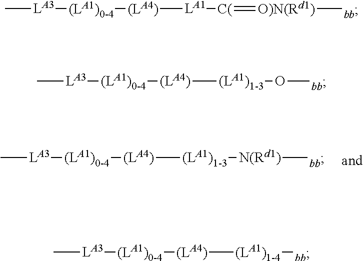

wherein bb represents the point of attachment to Ring C; and R^{d1} is —H or C₁₋₃ alkyl.

In some embodiments of Formula (A), n1 is an integer from 5 to 9; and L is:

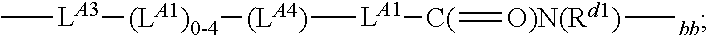

wherein bb represents the point of attachment to Ring C; and R^{d1} is —H or C₁₋₃ alkyl.

In some embodiments of Formula (A), n1 is an integer from 5 to 9; and L is:

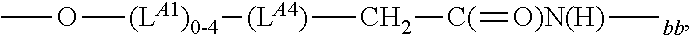

wherein bb represents the point of attachment to Ring C.

In some embodiments of Formula (A), n1 is an integer from 5 to 9; and L is:

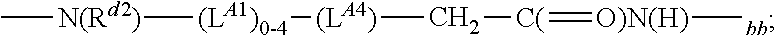

wherein bb represents the point of attachment to Ring C; and R^{d2} is —H or C₁₋₃ alkyl. For example, R^{d2} can be —H or methyl.

In some embodiments of Formula (A), L^{A4} is a 5-12 membered nitrogen-containing heterocyclylene optionally substituted with 1-3 R^{L4}.

In some embodiments of Formula (A), each R^{L4} is an independently selected R^{a}.

In some embodiments of Formula (A), each R^{L4} is independently selected from the group consisting of: —F, —CN, —OH, C₁₋₃ alkoxy, and C₁₋₃ alkyl optionally substituted with 1-3 —F.

In some embodiments of Formula (A), L^{A4} is a monocyclic 5-6 membered nitrogen-containing heterocyclylene optionally substituted with 1-3 R^{L4}, wherein each R^{L4} is independently selected from the group consisting of: —F, —CN, —OH, C₁₋₃ alkoxy, and C₁₋₃ alkyl optionally substituted with 1-3 —F.

In some embodiments of Formula (A), L^{A4} is

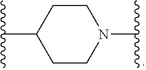 [SMILES: *C1CCN(*)CC1]

In some embodiments of Formula (A), L^{A4} is

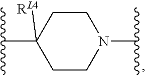

wherein R^{L4} is —F or —OH (e.g., —OH).

In some embodiments of Formula (A), L^{A4} is a bicyclic 7-12 membered nitrogen-containing heterocyclylene optionally substituted with 1-3 R^{L4}, wherein each R^{L4} is independently selected from the group consisting of: —F, —CN, —OH, C₁₋₃ alkoxy, and C₁₋₃ alkyl optionally substituted with 1-3 —F.

In some embodiments of Formula (A), L^{A4} is a spirocyclic bicyclic 7-12 membered nitrogen-containing heterocyclylene optionally substituted with 1-3 R^{L4}, wherein each R^{L4} is independently selected from the group consisting of: —F, —CN, —OH, C₁₋₃ alkoxy, and C₁₋₃ alkyl optionally substituted with 1-3 —F.

In some embodiments of Formula (A), L^{A4} is selected from the group consisting of:

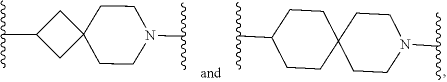 [SMILES: *C1CC2(CCN(*)CC2)C1 ; *C1CCC2(CC1)CCN(*)CC2]

each of which is optionally substituted with 1-3 R^{L4}, wherein each R^{L4} is independently selected from the group consisting of: —F, —CN, —OH, C₁₋₃ alkoxy, and C₁₋₃ alkyl optionally substituted with 1-3 F. For example, L^{A4} can be

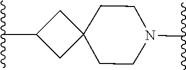 [SMILES: *C1CC2(CCN(*)CC2)C1]

In some embodiments of Formula (A), L^{A4} is a bridged or fused bicyclic 7-12 membered nitrogen-containing heterocyclylene optionally substituted with 1-3 R^{L4}, wherein each R^{L4} is independently selected from the group consisting of: —F, —CN, —OH, C₁₋₃ alkoxy, and C₁₋₃ alkyl optionally substituted with 1-3 —F.

In some embodiments of Formula (A), each L^{A1} is —CH₂—.

In some embodiments of Formula (A), one L^{A1} is —CHR^{L}₂— or —C(R^{L})₂—, wherein each R^{L} is independently —F or C₁₋₃ alkyl optionally substituted with 1-3 —F; and each remaining L^{A1} is —CH₂—. For example, one L^{A1} can be —CHMe-; and each remaining L^{A1} can be —CH₂—.

In some embodiments, the compounds of Formula (A) are selected from the group consisting of the compounds depicted in Table C3, or pharmaceutically acceptable salts thereof.

TABLE C3

| No. | Compound Structure |
|---|---|
| 2001 | 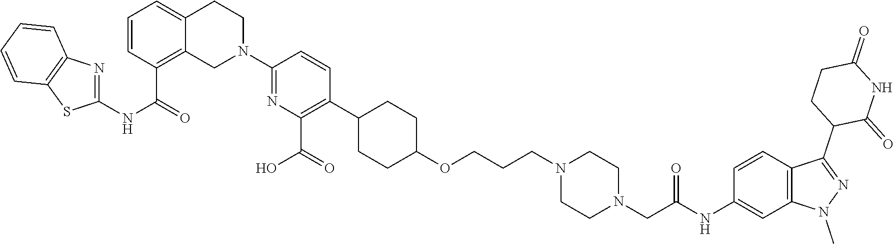 [SMILES: Cn1nc(C2CCC(=O)NC2=O)c2ccc(NC(=O)CN3CCN(CCCOC4CCC(c5ccc(N6CCc7cccc(C(=O)Nc8nc9ccccc9s8)c7C6)nc5C(=O)O)CC4)CC3)cc21] |
| 2001a | 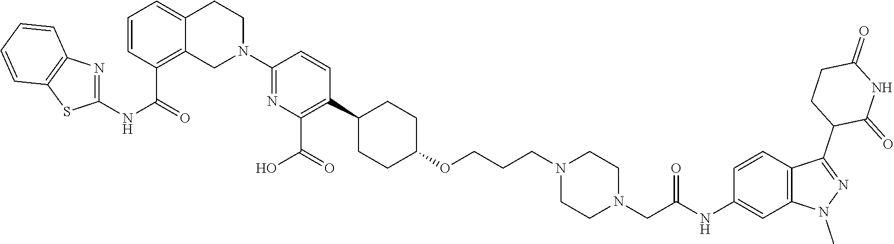 [SMILES: Cn1nc(C2CCC(=O)NC2=O)c2ccc(NC(=O)CN3CCN(CCCO[C@H]4CC[C@H](c5ccc(N6CCc7cccc(C(=O)Nc8nc9ccccc9s8)c7C6)nc5C(=O)O)CC4)CC3)cc21] |
| 2002 | 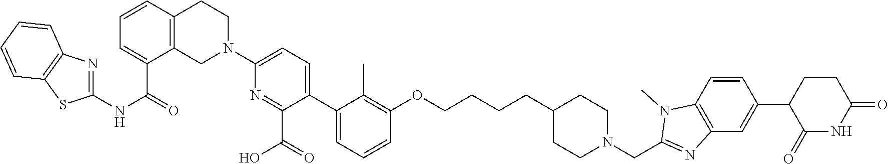 [SMILES: Cc1c(OCCCCC2CCN(Cc3nc4cc(C5CCC(=O)NC5=O)ccc4n3C)CC2)cccc1-c1ccc(N2CCc3cccc(C(=O)Nc4nc5ccccc5s4)c3C2)nc1C(=O)O] |
| 2003 | 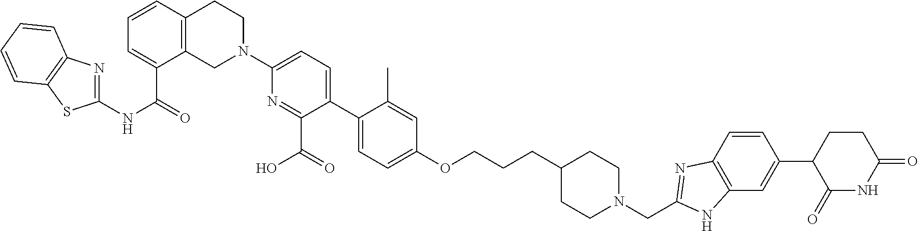 [SMILES: Cc1cc(OCCCC2CCN(Cc3nc4ccc(C5CCC(=O)NC5=O)cc4[nH]3)CC2)ccc1-c1ccc(N2CCc3cccc(C(=O)Nc4nc5ccccc5s4)c3C2)nc1C(=O)O] |

Certain examples of Formula (I), (II), or (A) compounds were synthesized using methods involving resolution of stereoisomeric mixture(s) (e.g., SFC separation of stereoisomers). In Table C1, C2, or C3, the resolved stereogenic centers in these compounds are labelled with the “or 1” or “or 2” enhanced stereochemical notations. In some instances, the stereoisomeric resolutions were performed during the last step of the synthesis, thereby providing the individual stereoisomers of the Formula (I), (II), or (A) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I), (II), or (A) compounds as separate stereoisomers. Methods of resolution and correlation between resolved intermediates and Formula (I), (II), or (A) compounds are disclosed in the examples herein. A person of ordinary skill in the art would understand that, under either approach for stereoisomeric resolution, stereoisomers having both (R)- and (S)-configurations at a resolved stereogenic center are provided. See Table C4, wherein Table C1, C2, and C3 compounds whose stereoisomers contain the or 1 notations are provided in non-stereogenic form, followed by the respective stereoisomers having the (R)- and (S)-configurations.

TABLE C4

| No. | Compound Structure |
|---|---|
| 130 | 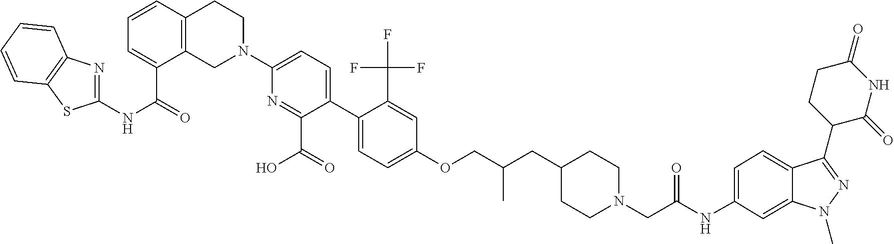 [SMILES: CC(COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| | 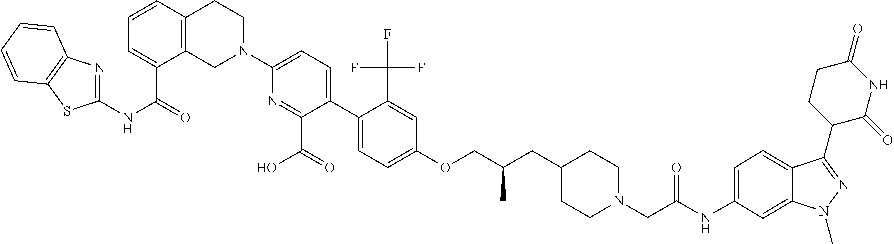 [SMILES: C[C@@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| | 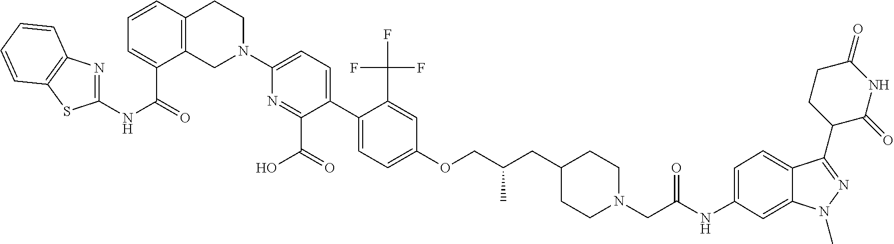 [SMILES: C[C@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1] |
| 133 | 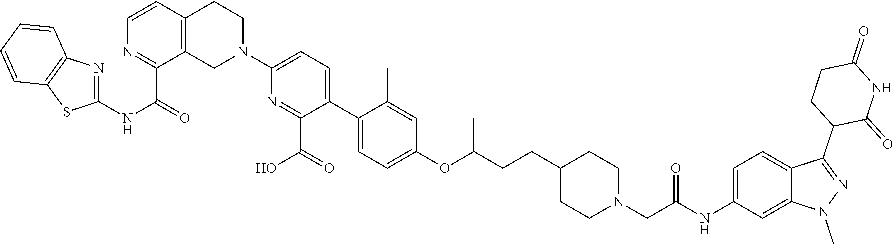 [SMILES: Cc1cc(OC(C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| | 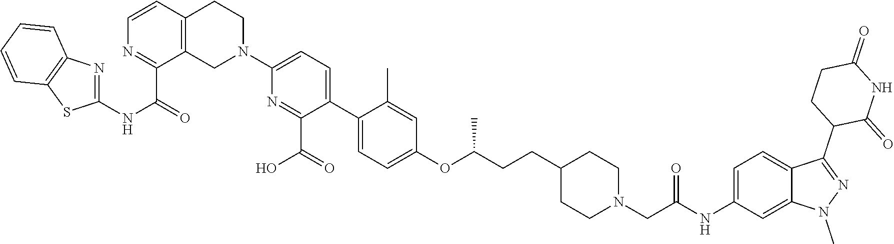 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| | 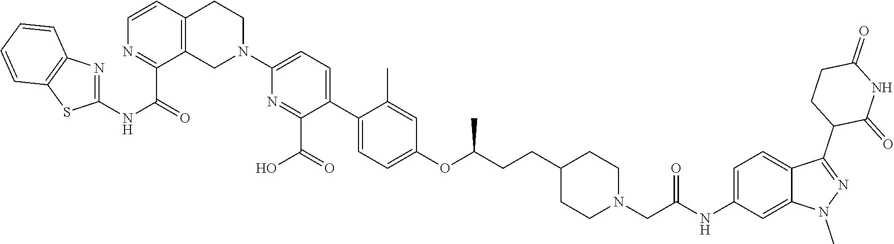 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 138 | 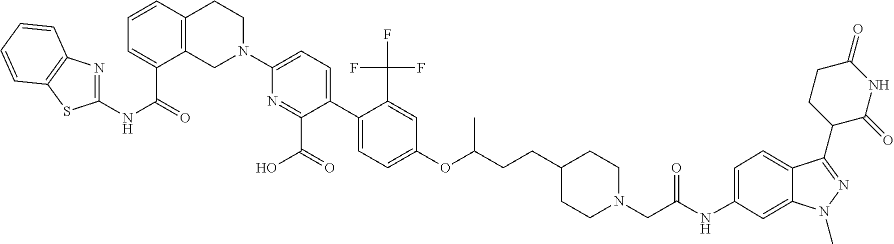 [SMILES: CC(CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| | 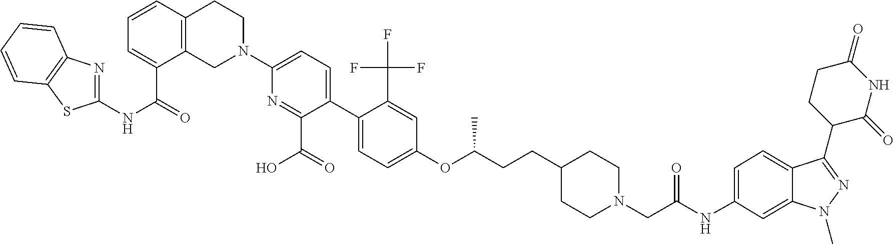 [SMILES: C[C@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| | 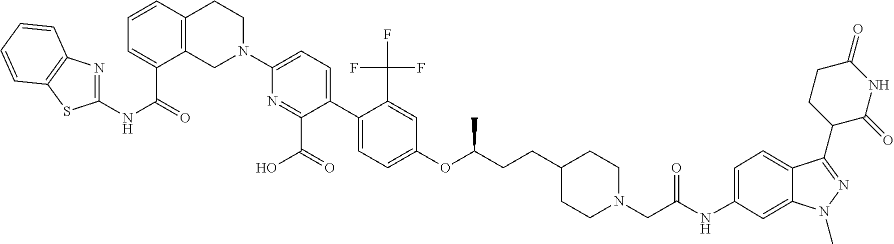 [SMILES: C[C@@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1] |
| 140 | 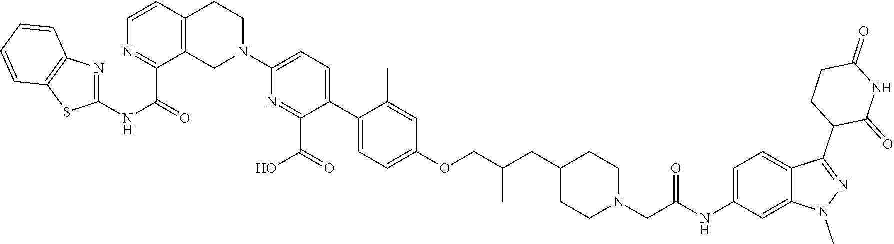 [SMILES: Cc1cc(OCC(C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| | 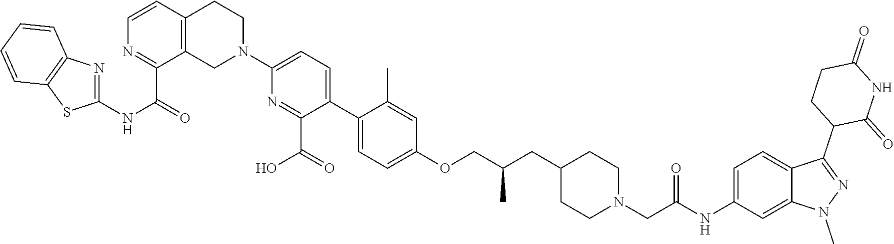 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| | 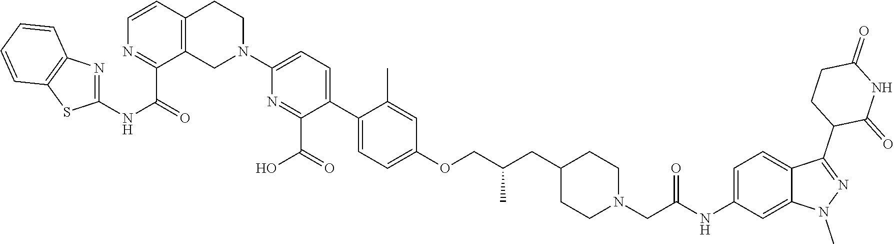 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O] |
| 223 | 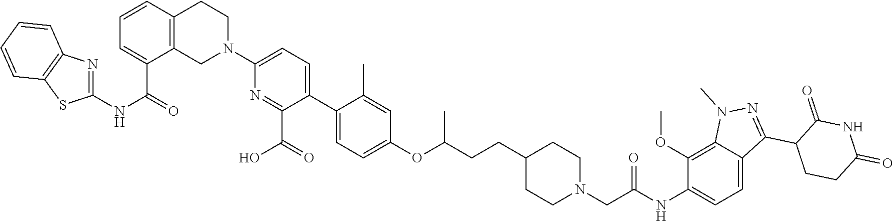 [SMILES: COc1c(NC(=O)CN2CCC(CCC(C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| | 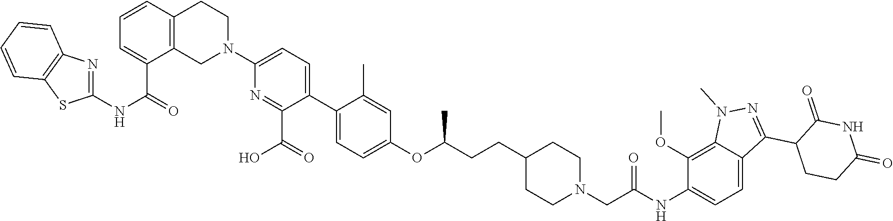 [SMILES: COc1c(NC(=O)CN2CCC(CC[C@H](C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| | 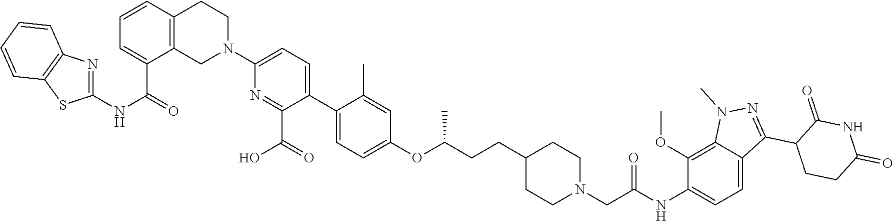 [SMILES: COc1c(NC(=O)CN2CCC(CC[C@@H](C)Oc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12] |
| 224 | 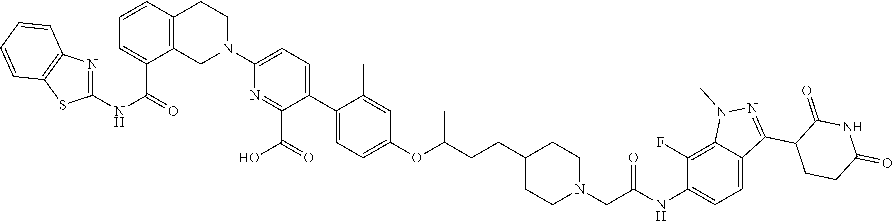 [SMILES: Cc1cc(OC(C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| | 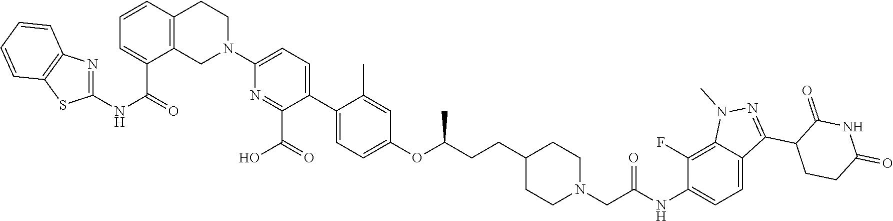 [SMILES: Cc1cc(O[C@@H](C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| | 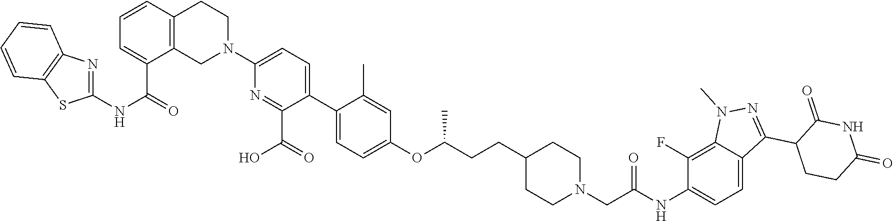 [SMILES: Cc1cc(O[C@H](C)CCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| 225 | 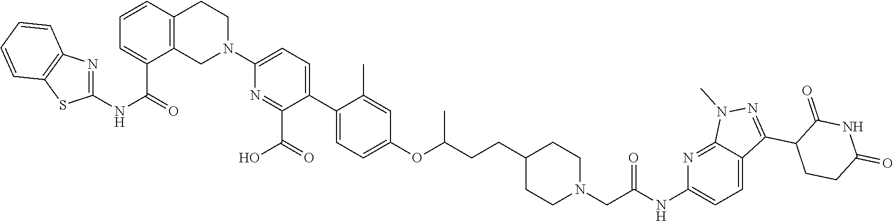 [SMILES: Cc1cc(OC(C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |
| | 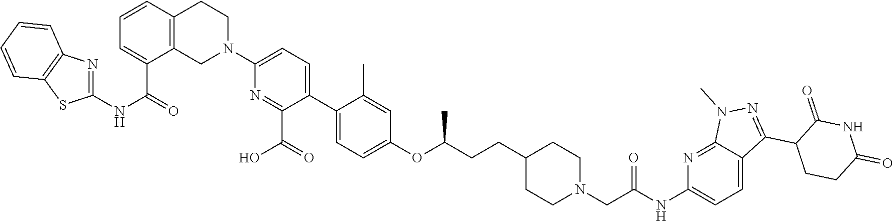 [SMILES: Cc1cc(O[C@@H](C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |
| | 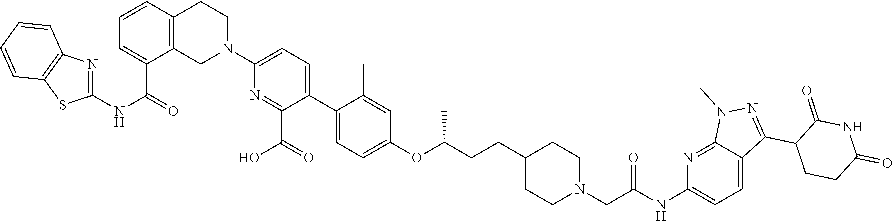 [SMILES: Cc1cc(O[C@H](C)CCC2CCN(CC(=O)Nc3ccc4c(C5CCC(=O)NC5=O)nn(C)c4n3)CC2)ccc1-c1ccc(N2CCc3cccc(C(=O)Nc4nc5ccccc5s4)c3C2)nc1C(=O)O] |
| 1001 | 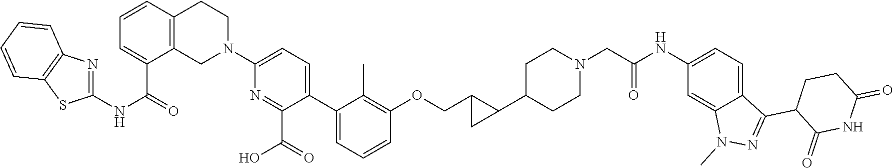 [SMILES: Cc1c(OCC2CC2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 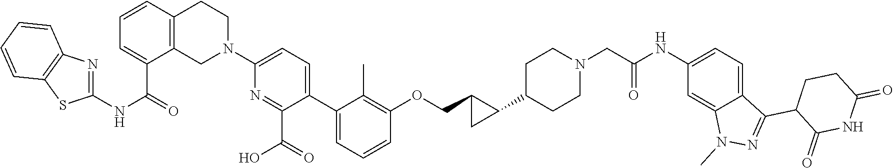 [SMILES: Cc1c(OC[C@@H]2C[C@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 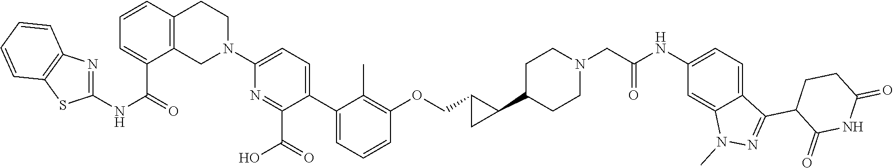 [SMILES: Cc1c(OC[C@H]2C[C@@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 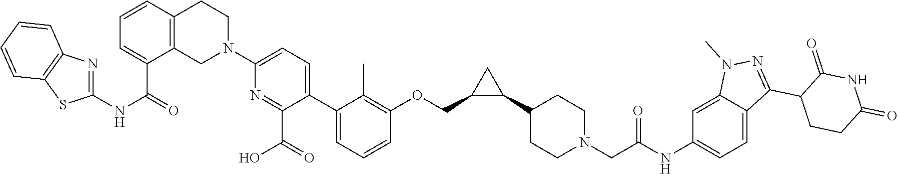 [SMILES: Cc1c(OC[C@H]2C[C@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 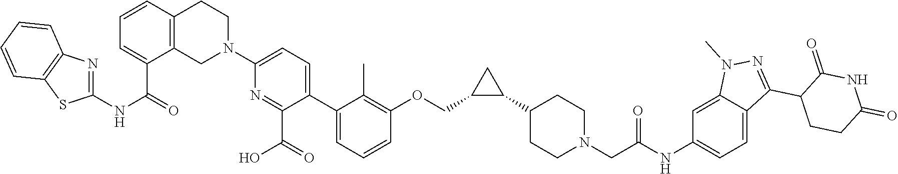 [SMILES: Cc1c(OC[C@@H]2C[C@@H]2C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 1002 | 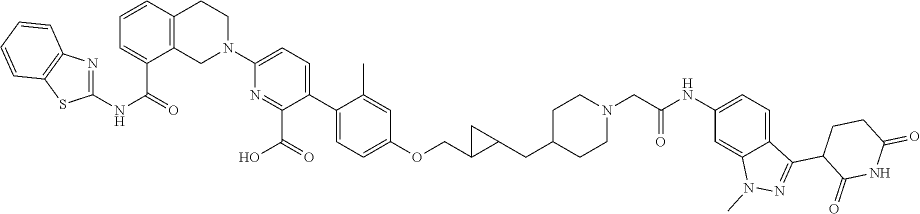 [SMILES: Cc1cc(OCC2CC2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 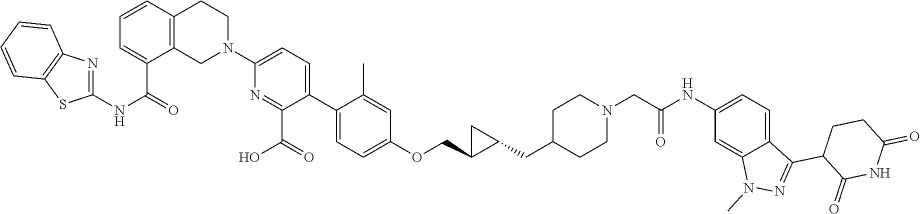 [SMILES: Cc1cc(OC[C@H]2C[C@@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 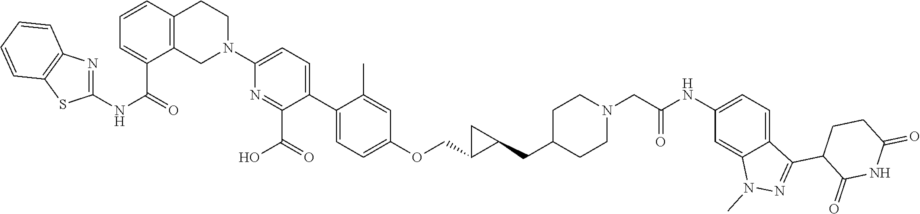 [SMILES: Cc1cc(OC[C@@H]2C[C@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 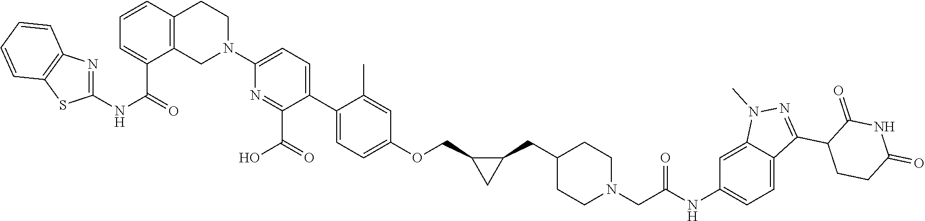 [SMILES: Cc1cc(OC[C@@H]2C[C@@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 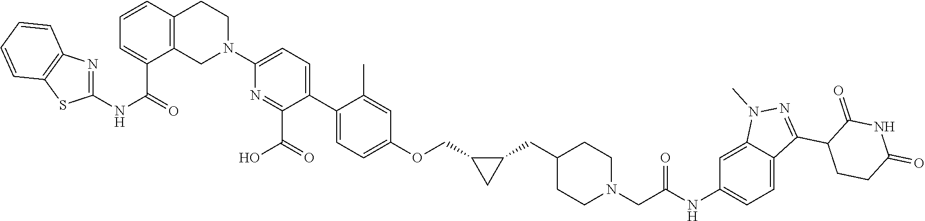 [SMILES: Cc1cc(OC[C@H]2C[C@H]2CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1003 | 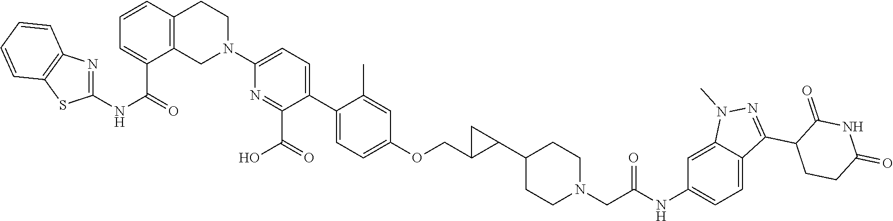 [SMILES: Cc1cc(OCC2CC2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 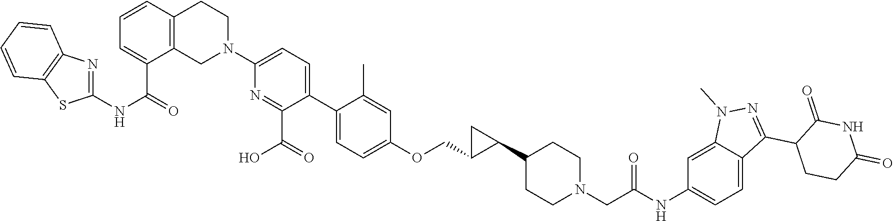 [SMILES: Cc1cc(OC[C@@H]2C[C@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 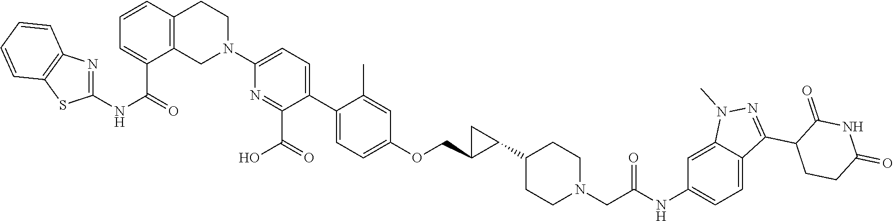 [SMILES: Cc1cc(OC[C@H]2C[C@@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 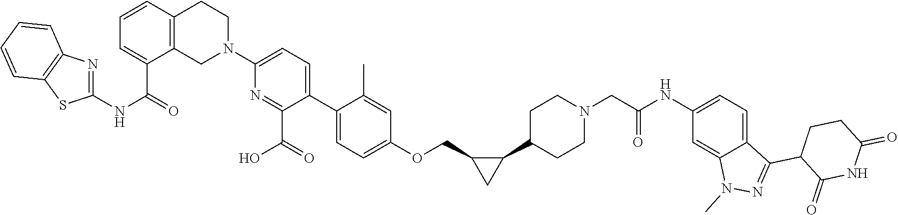 [SMILES: Cc1cc(OC[C@@H]2C[C@@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 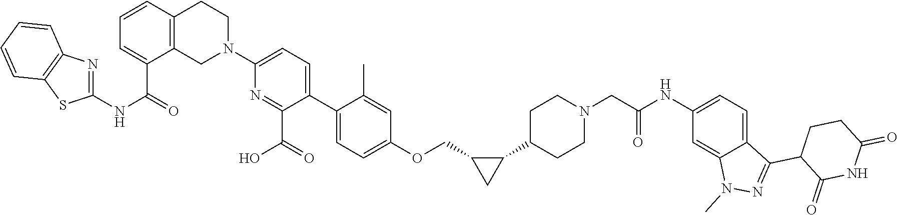 [SMILES: Cc1cc(OC[C@H]2C[C@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 1004 | 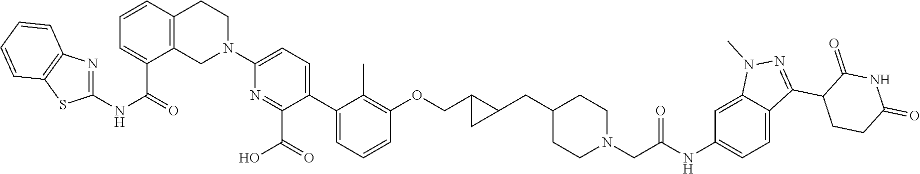 [SMILES: Cc1c(OCC2CC2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 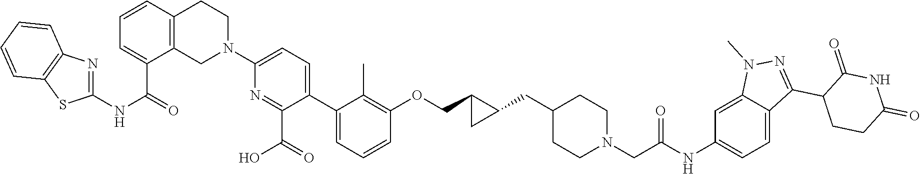 [SMILES: Cc1c(OC[C@@H]2C[C@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 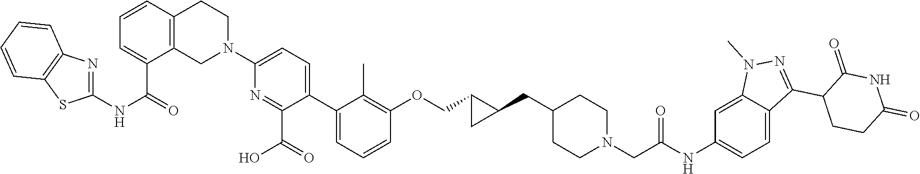 [SMILES: Cc1c(OC[C@H]2C[C@@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 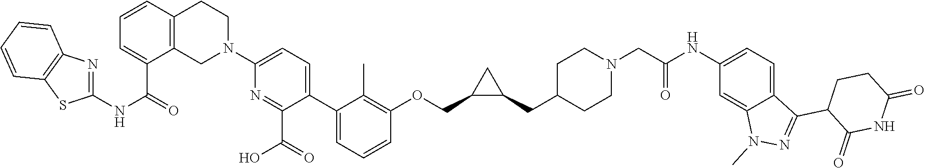 [SMILES: Cc1c(OC[C@H]2C[C@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 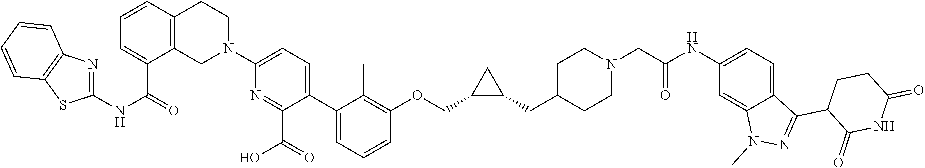 [SMILES: Cc1c(OC[C@@H]2C[C@@H]2CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |

In some embodiments, the compounds of Formula (I) (e.g., Formula (I-1) (e.g., (I-a1), (I-b1), (I-b3), (I-c1), (I-d1), (I-e1), (I-e3), (I-f1), (1-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, reduce cell viability in a cell line expressing a BCL-X_{L} protein with an EC₅₀ of less than 1 μM (e.g., less than 750 nM, less than 500 nM, or less than 200 nM). In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A) reduce cell viability in a cell line expressing the BCL-X_{L} protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL-X_{L} protein with an EC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) (e.g., Formula (I-1) (e.g., (I-a1), (I-b1), (I-b3), (I-c1), (I-d1), (I-e1), (I-e3), (I-f1), (1-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, induce degradation of a BCL-X_{L} protein in a cell line expressing the BCL-X_{L} protein with a DC₅₀ of less than 1 μM (e.g., less than 750 nM, less than 500 nM, or less than 200 nM). In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-hi), (I-i1), or (I-i3)) or Formula (I-2) (e.g., (I-a2), (I-b2), (I-b4), (I-c2), (I-d2), (I-e2), (I-e4), (I-2), (I-g2), (I-g4), (I-h2), or (I-i2))), Formula (II) (e.g., Formula (II-a1) or (II-a2)), or Formula (A), or a pharmaceutically acceptable salt thereof, induce degradation of a BCL-X_{L} protein in a cell line expressing the BCL-X_{L} protein with a DC₅₀ of less than 200 nM (e.g., less than 150 nM, less than 200 nM, less than 100 nM, less than 10 nM, less than 1 nM). For example, the compounds can induce degradation of a BCL-X_{L} protein in a cell line expressing the BCL-X_{L} protein with a DC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, induce degradation of a BCL-X_{L} protein in a cell line expressing the BCL-X_{L} protein with an EC₅₀ of less than 1 μM (e.g., less than 750 nM, less than 500 nM, or less than 200 nM). In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (I-a1) or (II-a2)), or Formula (A), or a pharmaceutically acceptable salt thereof, induce degradation of a BCL-X_{L} protein in a cell line expressing the BCL-X_{L} protein with an EC₅₀ of less than 200 nM (e.g., less than 150 nM, less than 200 nM, less than 100 nM, less than 10 nM, less than 1 nM). For example, the compounds can induce degradation of a BCL-X_{L} protein in a cell line expressing the BCL-X_{L} protein with an EC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) (e.g., Formula (I-1) (e.g., (I-a1), (I-b1), (I-b3), (I-c1), (I-d1), (I-e1), (I-e3), (I-f1), (1-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or pharmaceutically acceptable salts thereof, induce degradation of a BCL-X_{L} protein in a cell line expressing the BCL-X_{L} protein with a Yₘᵢₙ of less than 70% (e.g., less than 50%, less than 30%, less than 20%, or less than 10%). In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (I-a1) or (II-a2)), or Formula (A), or a pharmaceutically acceptable salt thereof, induce degradation of a BCL-X_{L} protein in a cell line expressing the BCL-X_{L} protein with a Yₘᵢₙ of less than 50% (e.g., less than 40%, less than 30%, less than 20%, less than 10%, or less than 5%). In some embodiments, the compounds of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (I-a1) or (II-a2)), or Formula (A), or a pharmaceutically acceptable salt thereof, induce degradation of a BCL-X_{L} protein in a cell line expressing the BCL-X_{L} protein with a Yₘᵢₙ of less than 30% (e.g., less than 25%, less than 20%, less than 15%, less than 10%, or less than 5%). For example, the compounds can induce degradation of a BCL-X_{L} protein in a cell line expressing the BCL-X_{L} protein with a Yₘᵢₙ of about 1% to about 70% (e.g., about 5% to about 50% or about 10% to about 30%).

Also provided herein is a BCL-X_{L} protein non-covalently bound with a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or a pharmaceutically acceptable salt thereof.

Also provided herein is a ternary complex comprising a BCL-X_{L} protein, a compound of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-f2), (I-g2), (I-g4), (I-h2), or (I-i2))), Formula (II) (e.g., Formula (II-a1) or (II-a2)), or Formula (A), or a pharmaceutically acceptable salt thereof, and a CRBN protein, or a portion thereof.

### Chemical Definitions

The term “halo” refers to fluoro (F), chloro (Cl), bromo (Br), or iodo (I).

The term “oxo” refers to a divalent doubly bonded oxygen atom (i.e., “═0”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

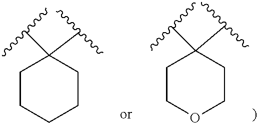 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

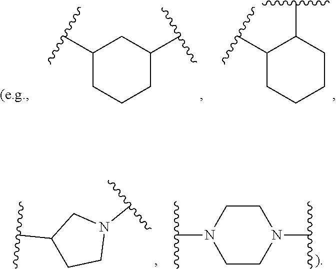 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic (e.g., fused, bridged, or spirocyclic bi-, tri-, or polycyclic) saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Cycloalkyl also includes spirocyclic rings (e.g., spirocyclic bicycle wherein two rings are connected through just one atom). Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

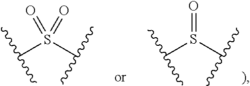

and P (inclusive of oxidized forms such as:

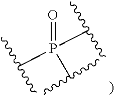 [SMILES: *P(*)(*)=O]

(e.g., N, O, and S (inclusive of oxidized forms such as:

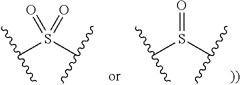

and at least one ring in the system is aromatic (but does not have to be a ring which contains a heteroatom, e.g. tetrahydroisoquinolinyl, e.g., tetrahydroquinolinyl). In some embodiments, heteroaryl groups contain 1-4 (e.g., 1, 2, or 3) ring heteroatoms each independently selected from the group consisting of N, O, and S (inclusive of oxidized forms such as:

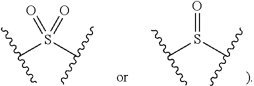

Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

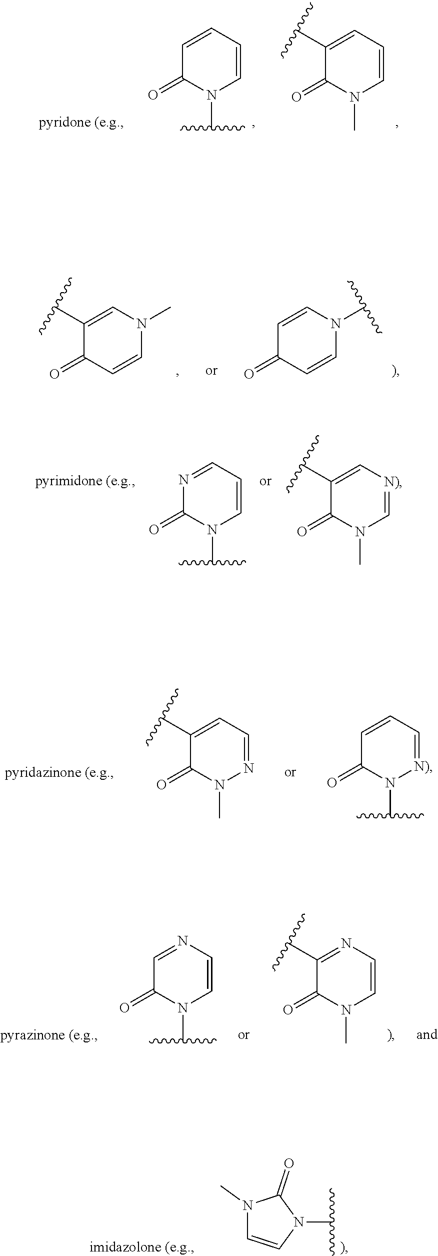

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic (e.g., fused, bridged, or spirocyclic bi-, tri-, or polycyclic) saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, the heteroatoms selected from O, N, S (inclusive of oxidized forms such as:

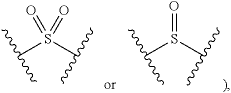

and P (inclusive of oxidized forms such as:

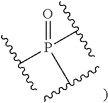 [SMILES: *P(*)(*)=O]

(e.g., O, N, and S (inclusive of oxidized forms such as:

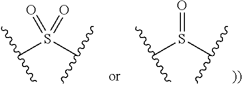

(e.g. carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. In some embodiments, heterocyclyl groups contain 1-4 (e.g., 1, 2, or 3) ring heteroatoms each independently selected from the group consisting of N, O, and S (inclusive of oxidized forms such as:

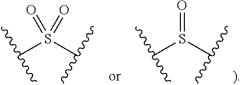

The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl. Heterocyclyl may include multiple fused and/or bridged rings. Non-limiting examples of fused/bridged heterocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrole, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

A nitrogen-containing heterocyclyl as used herein refers to a heterocyclyl having 1-2 ring nitrogen atoms and 0-2 additional ring heteroatoms selected from the group consisting of O and S (inclusive of oxidized such as:

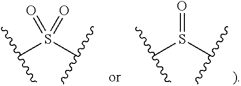

The nitrogen-containing heterocyclyl can be monocyclic, bicyclic, or polycyclic as defined elsewhere herein. Examples of monocyclic nitrogen-containing heterocyclyl include azetidinyl, pyrrolidinyl, piperidinyl, piperazinyl, morpholinyl, and the like. Examples of bicyclic nitrogen-containing heterocyclyl include 7-azaspiro[3.5]nonyl, 1,7-diazaspiro[4.5]decyl, 3-oxa-7,9-diazabicyclo[3.3.1]nonanyl, 2,6-diazaspiro[3.3]heptanyl, and the like.

As used herein, when a ring is described as being “partially unsaturated”, it means the ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

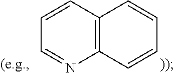

(ii) a single ring atom (spiro-fused ring systems)

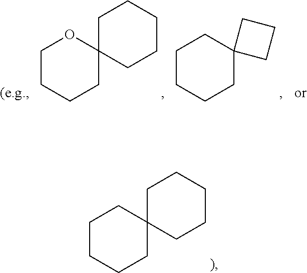

or (iii) a contiguous array of ring atoms (bridged ring systems having all bridge lengths >0)

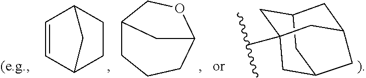

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

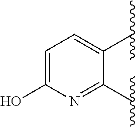 [SMILES: *c1ccc(O)nc1*]

encompasses the tautomeric form containing the moiety:

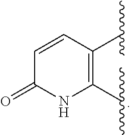 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

Certain combinations of heteroatoms (e.g., N, O, S, or halo) define compounds which are less stable under physiological conditions. Examples include (1) compounds containing acetal or aminal linkages; (2) compounds containing acyclic N—O, N—N, or N—S(O)₀ bonds; and (3) compounds containing O—O, O—S(O)₀₋₂, N-halo, O-halo, and S(O)₀₋₂-halo bonds. Accordingly, such compounds are less preferred. As used herein, “acyclic bonds” mean chemical bonds that are not part of a ring. Examples include the N—O bond in

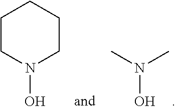

For avoidance of doubt, acyclic N—O, N—N, or N—S(O)₀ bonds (i.e., those bonds that are not part of a ring (e.g., in

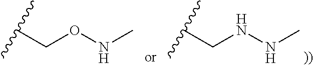

are less preferred, but compounds provided herein can include N—O, N—N, or N—S(O)₀ bonds that form part of a ring (e.g., the N—N bond in

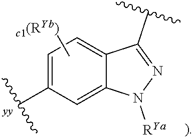

### Methods of Treatment

### Indications

Provided herein are methods for inducing degradation of a BCL-X_{L} protein. For example, provided herein are compounds capable of inducing degradation of a BCL-X_{L} protein useful for treating or preventing cancers. See, e.g., Guo, et al. Aging (Albany NY) 13.15 (2021): 19750, doi: 10.18632/aging.203386; Park, et al. Proceedings of the National Academy of Sciences 112.40 (2015): 12492-12497, doi: 10.1073/pnas.1507491112; Zhang, et al. Molecular Cancer 14.1 (2015): 1-9, doi: 10.1186/s12943-015-0397-y; Beroukhim, et al. Nature 463.7283 (2010): 899-905, doi: 10.1038/nature08822.

The term “compound(s) provided herein” refers to compound(s) of Formula (I) (e.g., Formula (I-1) (e.g., (I-a1), (I-b1), (I-b3), (I-c1), (I-d1), (I-e1), (I-e3), (I-f1), (I-g1), (I-g3), (I-h1), (I-i1), or (I-i3)) or Formula (I-2) (e.g., (I-a2), (I-b2), (I-b4), (I-c2), (I-d2), (I-e2), (I-e4), (I-2), (I-g2), (I-g4), (I-h2), or (I-i2))), Formula (II) (e.g., Formula (II-a1) or (II-a2)), or Formula (A) as disclosed herein.

The effect of protein degradation typically increases over time, and the appearance of degradation (e.g., as expressed by the percentage degradation compared to a control, or the parameters Yₘᵢₙ, DC₅₀, EC₅₀, and/or Dₘₐₓ) is affected by the resynthesis rate of the protein Accordingly, degradation can be examined after a specified period of time, such as 6 hours, 12 hours, 18 hours, 1 day, 2 days, 3 days, or more. For example, degradation can be expressed as the percent degradation after 24 hours.

Exemplary assays for validating the degradation-inducing mechanism of a compound as provided herein are known in the art and are described, for example, in International Publication No. WO 2019/144117 and Wu, et al. Nature Structural & Molecular Biology 27.7 (2020): 605-614, doi: 10.1038/s41594-020-0438-0.

Degradation assays can be used to quantify both on- and off-target degradation-inducing effects of compounds, such as those compounds provided herein. Exemplary assays include quantitative immunoblotting, other immunoassays (e.g., MesoScale Discovery (MSD) immunoassays), homogenous time resolved florescence (HTRF), and HiBiT. In some embodiments, cells can be contacted with a compound provided herein, or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL-X_{L} protein, and the amount of fluorescence observed when the complementary LgBiT peptide is added can be compared between cells treated with a compound provided herein, or a pharmaceutically acceptable salt thereof, and a control (e.g., a DMSO-treated control). See, for instance, Example B1 and Example B2. See also, e.g., International Publication Nos. WO 2020/163823 and WO 2019/144117. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CK1α).

See also the assays described in International Publication Nos. WO 2023/044046; WO 2022/169780; WO 2021/222114; WO 2021/146536; WO 2021/078301; WO 2021/007307; WO 2020/163823; WO 2019/144117; WO 2017/184995, and Khan, et al. Nature Medicine 25.12 (2019): 1938-1947, doi: 10.1038/s41591-019-0668-z; Balachander, et al. Clinical Cancer Research 26.24 (2020): 6535-6549, doi: 10.1158/1078-0432.CCR-20-0863.

Binding affinity of a compound provided herein, or a pharmaceutically acceptable salt thereof to BCL-X_{L} can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a binding IC₅₀ value can be determined in a fluorescence polarization assay using a fluorescently labeled BH3-only peptide (e.g., BAD or BAX) as the competitor. As another example, a binding Kᵢ value can be determined using a time resolved-fluorescence resonance energy transfer (TR-FRET) assay using a fluorescently labeled BH3-only peptide (e.g., BAK) and a fluorescently labeled antibody that binds to BCL-X_{L}, where the fluorophores are a FRET pair, and using a compound provided herein, or a pharmaceutically acceptable salt thereof, as provided herein as a competitor for the BH3-only peptide. See, e.g., U.S. Patent Publication Nos. US 2007/0027135, US 2010/305122, and US 2013/096120.

Another exemplary assay for evaluating the affinity of a compound provided herein, or a pharmaceutically acceptable salt thereof, includes using a competition assay with recombinant BCL-X_{L} protein. For example, purified recombinant affinity-tagged (e.g., His-tagged) BCL-X_{L} protein can be incubated with various concentrations of a compound provided herein, or a pharmaceutically acceptable salt thereof, and a fixed concentration of affinity-tagged (e.g., biotin-tagged) BAD protein. After a period of incubation, FRET acceptor beads with a complementary affinity tag (e.g., His-acceptor beads) and FRET donor beads (e.g., streptavidin-tagged donor beads) can be added to the mixture, and a FRET reaction can be used to determine an inhibition constant of the compound provided herein, or pharmaceutically acceptable salt thereof. For example, an AlphaLISA competitive assay can be performed. See, e.g., International Publication No. WO 2019/144117.

The ability of a compound provided herein, or a pharmaceutically acceptable salt thereof, to inhibit BCL-X_{L} can be determined using an IC₅₀ value. A compound with a lower IC₅₀ value, as determined under substantially similar conditions, is a more potent inhibitor relative to a compound with a higher IC₅₀ value. One way that inhibition of BCL-X_{L} can be measured is measuring the interruption of the formation of a complex of BCL-X_{L} with a BH3-only peptide (e.g., BIM). For example, an electrochemiluminescence-based sandwich ELISA assay (e.g., a Meso Scale Discovery (MSD)-ELISA assay) can be used. See, for example, Phillips, D. C., et al. Blood Cancer Journal 5.11 (2015): e368-e368, doi: 10.1038/bcj.2015.88 and Xiao, Yu, et al. Molecular Cancer Therapeutics 14.8 (2015): 1837-1847, doi: 10.1158/1535-7163.MCT-14-0928. In such assays, cells expressing BCL-X_{L} can be incubated with a compound provided herein, or a pharmaceutically acceptable salt thereof for a period of time, lysed, and then evaluated in the assay. A tagged anti-BCL-X_{L} antibody (e.g., a biotin-tagged anti-BCL-X_{L} antibody) can be immobilized on an assay plate (e.g., a streptavidin assay plate), and then the lysate can be applied to pull down BCL-X_{L}. An anti-BIM antibody (e.g., a rabbit anti-BIM antibody) can be introduced, followed by addition of a detection antibody (e.g., a sulfo-tagged goat anti-rabbit antibody) and measurement of the detection antibody. As another example, interruption of the formation of a complex of BCL-X_{L} with a BH3-only peptide (e.g., BIM) can be measured using a mammalian two-hybrid assay. In such assays, a plasmid encoding the ‘bait’ and ‘prey’ fusion proteins (e.g., the DNA binding domain of GAL4 fused to BCL-X_{L} and the transcriptional activation domain of VP16 fused to BIM) can be introduced to cells (e.g., HeLa cells) stably expressing a GAL4-luciferase reporter. A compound provided herein, or a pharmaceutically acceptable salt thereof, can be added to the cells in culture, incubated, and the luciferase activity can be measured. See, for example, Souers, Andrew J., et al. Nature Medicine 19.2 (2013): 202-208, doi: 10.1038/nm.3048.

Additional methods of evaluating the binding of a compound to BCL-X_{L} protein or the inhibition of a BCL-X_{L} protein are described in, for example, U.S. Patent Publication Nos. US 2007/027135; US 2010/305122, and US 2013/096120.

Potency of degradation by a compound provided herein, or a pharmaceutically acceptable salt thereof, as provided herein can be determined by DC₅₀ value. As used herein, DC₅₀ refers to the concentration of the compound provided herein, or a pharmaceutically acceptable salt thereof, that results in a 50% decrease in the concentration of a protein (e.g., BCL-X_{L} protein) in a cell compared to the concentration of the protein before the cell is contacted with the compound provided herein, or a pharmaceutically acceptable salt thereof, or compared to the concentration of the protein in a cell not contacted with the compound provided herein, or a pharmaceutically acceptable salt thereof. A compound with a lower DC₅₀ value, as determined under substantially similar conditions, is a more efficient inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

Potency of degradation by a compound provided herein, or a pharmaceutically acceptable salt thereof, as provided herein can be determined by EC₅₀ value. As used herein, EC₅₀ refers to the concentration of the compound provided herein, or a pharmaceutically acceptable salt thereof that results in a 50% decrease in the concentration of a protein (e.g., BCL-X_{L} protein) relative to the trough concentration of the protein in a cell, when compared to the concentration of the protein before the cell is contacted with the compound provided herein, or a pharmaceutically acceptable salt thereof, or compared to the concentration of the protein in a cell not contacted with the compound provided herein, or a pharmaceutically acceptable salt thereof. A compound with a lower EC₅₀ value, as determined under substantially similar conditions, is a more potent compound relative to a compound with a higher EC₅₀ value. In some embodiments, an EC₅₀ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

Potency of degradation by a compound provided herein, or a pharmaceutically acceptable salt thereof, as provided herein can be determined by Yₘᵢₙ value. As used herein, Yₘᵢₙ refers to the ratio of trough concentration of a protein (e.g., BCL-X_{L} protein) in a cell compared to the concentration of the protein before the cell is contacted with the compound provided herein, or a pharmaceutically acceptable salt thereof, or compared to the concentration of the protein in a cell not contacted with the compound provided herein, or a pharmaceutically acceptable salt thereof, expressed as a percentage. As used herein, Dₘₐₓ is 1-Yₘᵢₙ. Yₘᵢₙ can be measured by a HiBiT assay (e.g., as described in Example B1). A compound with a lower Yₘᵢₙ value, as determined under substantially similar conditions, is a more potent inducer of degradation relative to a compound with a higher Yₘᵢₙ value. In some embodiments, a Yₘᵢₙ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

An exemplary assay for determining the potency of a compound provided herein, or a pharmaceutically acceptable salt thereof, includes measuring the effect of the compound provided herein, or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, MOLT4, RS4; 11, NCI-H146, EJM, HEK293T, HT1080, and/or H929. As an illustrative example, a 3D cell proliferation assay can include growing cells in a 3D medium, contacting the cells with a compound provided herein, or a pharmaceutically acceptable salt thereof, measuring the cellular proliferation using an appropriate reagent (e.g., CELLTITER-GLO® 3D), and then comparing the signal from an experiment with a compound provided herein, or a pharmaceutically acceptable salt thereof, to the signal from a control experiment (e.g., lacking the compound provided herein, or a pharmaceutically acceptable salt thereof). As another illustrative example, a 2D cell proliferation assay can include plating cells onto a growth surface, optionally letting the cells grow for a period of time, contacting the cells with a compound provided herein, or a pharmaceutically acceptable salt thereof, measuring the cellular proliferation using an appropriate reagent (e.g., CELLTITER-GLO®), and then comparing the signal from an experiment with a compound provided herein, or a pharmaceutically acceptable salt thereof, to the signal from a control experiment (e.g., lacking a compound provided herein, or a pharmaceutically acceptable salt thereof). In some embodiments, assessment degradation-mediated antiproliferation effects from compounds provided herein, CRBN knockout cells can be used in a similar cell viability assay. The CRBN knockout cells can be generated from parental cells (e.g., MOLT-4 cells) using Crispr gene editing technology. CRBN-targeted single guide RNA (sgRNA) and Cas9 protein can be purchased from commercial suppliers, and Cas9-sgRNA ribonucleoproteins (RNPs) can electroporated into parental cells (e.g., MOLT-4 cells) using a kit (e.g., a Neon NxT Electroporation Kit (Invitrogen)). Cell viability can be assessed for the CRBN knockout cells in the same way as for parental cells (e.g., MOLT-4 cells). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts. See, for instance, Example B3.

A cell viability assay can be used to measure the effect of a compound provided herein, or a pharmaceutically acceptable salt thereof, on cell death. For example, cells expressing BCL-X_{L} protein (e.g., MOLT-4 cells) can be incubated with various concentrations of a compound provided herein, or a pharmaceutically acceptable salt thereof, then exposed to a detection reagent (e.g., a CELLTITER-GLO® Cell Viability Assay kit) to determine cell viability.

An exemplary assay for determining the mechanism of cell death using of a compound provided herein, or a pharmaceutically acceptable salt thereof, includes measuring the effect of the compound provided herein, or a pharmaceutically acceptable salt thereof, on one or more markers of a mechanism of cell death (e.g., apoptosis). Exemplary markers of apoptosis include caspase induction (e.g., caspase 3/7 induction) and annexin V staining. Such assays may also be used as determinants of cell viability. For example, cells expressing BCL-X_{L} protein (e.g., MOLT-4 cells) can be incubated with various concentrations of a compound provided herein, or a pharmaceutically acceptable salt thereof, and relative caspase activity can be assessed using a luciferase substrate that is activated by caspase 3/7 (e.g., using the CASPASE-GLO® 3/7 assay). As another example, cells expressing BCL-X_{L} protein (e.g., MOLT-4 cells) can be incubated with various concentrations of a compound provided herein, or a pharmaceutically acceptable salt thereof, and relative caspase activity can be assessed using a dye that is coupled to an activated caspase motif (e.g., INCUCYTE® Caspase 3/7 Green Apoptosis Assay Reagent), followed by analysis using a platform for live cell imaging (e.g., an INCUCYTE® SX5 Live-Cell Analysis Instrument). As another example, cells expressing BCL-X_{L} protein (e.g., MOLT-4 cells) can be incubated with various concentrations of a compound provided herein, or a pharmaceutically acceptable salt thereof, and annexin V positivity can be assessed using phosphatidylserine dye (e.g., an INCUCYTE® Annexin V dye), followed by analysis using a platform for live cell imaging (e.g., an INCUCYTE® SX5 Live-Cell Analysis Instrument).

As another example, the potency and/or efficacy of a compound provided herein, or a pharmaceutically acceptable salt thereof, can be evaluated in an animal model, for example, a cell line-derived xenograft (CDX) model (e.g., using an established cancer cell line such as MOLT4, HEL, TF1, F36P, OCI-M1, OCI-M2, SET-2, CMK, M07E, UKE-1, or NCI-H1417), or a patient-derived xenograft (PDX) model. For example, a CDX or PDX model can be run in immunodeficient mice (e.g., athymic nude, outbred homozygous (e.g., Crl:NU(NCr)-Foxn1^{nu}) or Fox Chase SCID (CB17/Icr-Prkd^{scid}/IcrIcoCrl), mice). The mice can be female, 6-12 weeks told at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound provided herein, or a pharmaceutically acceptable salt thereof, (e.g., via IP or PO (oral) administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study, the Best Response can be calculated for each treatment arm. Best Response is defined as the minimum value of ΔVolumeₜ for t≥10 days. Best Responses between the control arm(s) and the treatment arm(s) can be compared to determine if the treatment(s) work better than the control(s). In some embodiments, tumor samples can also be collected at the end of each study and relevant proteins (e.g., BCL-X_{L}, BCL-2, BCL-W, MCL-1, BIM, BAX, and/or BAK) can be measured to determine if the treatment might have a better protein modulation profile compared to a control. In some embodiments, tumor samples can also be collected at the end of each study and analyzed for signaling pathway activity (e.g., via phosphoERK levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 5, 6 hours post dosing, and relevant proteins can be measured in the tumor samples and pharmacokinetic studies can be performed on the blood samples or a portion thereof (e.g., plasma).

In some embodiments, the PDX is a model of a myeloproliferative neoplasm (MPN) (e.g., CEL, CML, CNL, essential thrombocythemia (e.g., JAK2 mutant (e.g., JAK2 V617F mutant) essential thrombocythemia or JAK2 wild type essential thrombocythemia), polycythemia vera (e.g., JAK2 mutant (e.g., JAK2 V617F mutant) polycythemia vera or JAK2 wild type polycythemia vera), or myelofibrosis (e.g., primary myelofibrosis (e.g., JAK2 mutant (e.g., JAK2 V617F mutant) primary myelofibrosis or JAK2 wild type primary myelofibrosis), post-essential thrombocythemia myelofibrosis (e.g., JAK2 mutant (e.g., JAK2 V617F mutant) post-essential thrombocythemia myelofibrosis or JAK2 wild type post-essential thrombocythemia myelofibrosis), or post-polycythemia vera myelofibrosis (e.g., JAK2 mutant (e.g., JAK2 V617F mutant) post-polycythemia vera myelofibrosis or JAK2 wild type post-polycythemia vera myelofibrosis)))), a CRC (e.g., BRaf mutant CRC (e.g., Braf V600E CRC) or KRas mutant CRC (e.g., KRas G12C mutant CRC or KRas G12D CRC)), a SCLC (e.g., ASCL1 subtype SCLC or NEUROD1 subtype SCLC), a NSCLC (e.g., BRaf mutant NSCLC (e.g., Braf V600E NSCLC), an EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), a KRas mutant NSCLC (e.g., KRas G12C NSCLC)), a lung squamous cell carcinoma, a malignant pleural mesothelioma (e.g., a BAP1 mutant malignant pleural mesothelioma), a melanoma (e.g., Braf mutant melanoma (e.g., Braf V600E melanoma)), a breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), a lymphoma (e.g., a T cell lymphoma (e.g., anaplastic large T cell lymphoma, cutaneous T cell lymphoma, or peripheral T cell lymphoma), or a non-Hodgkin lymphoma (e.g., DLBCL, anaplastic large T cell lymphoma, cutaneous T cell lymphoma, or peripheral T cell lymphoma), a leukemia (e.g., T cell leukemia (e.g., a T-ALL (e.g., relapsed/refractory T-ALL)), post-MPN leukemia, M6-AML, M7-AML), a head and neck cancer, a pancreatic cancer, a bladder cancer, an ovarian cancer (e.g., BRCA1 mutant ovarian cancer or BRCA2 mutant ovarian cancer, HGSOC (e.g., BRCA1 mutant HGSOC or BRCA2 mutant HGSOC)), a cervical cancer, an intrahepatic cholangiocarcinoma, or a mesenchymal cancer (e.g., mesenchymal breast cancer or mesenchymal kidney cancer).

See, e.g., Khan, et al. Nature Medicine 25.12 (2019): 1938-1947, doi: 10.1038/s41591-019-0668-z; Balachander, et al. Clinical Cancer Research 26.24 (2020): 6535-6549, doi: 10.1158/1078-0432.CCR-20-0863.

The pharmacokinetic parameters of a compound provided herein, or a pharmaceutically acceptable salt thereof, can be evaluated in an animal model, for instance, a mouse model, a rat model, a dog model, or a nonhuman primate (e.g., cynomolgus monkey) model. An exemplary protocol includes the following. For example, pharmacokinetics (PK) studies can be conducted on animals (e.g., male or female CD-1 mice, Sprague Dawley rats, beagle dogs, or cynomolgus monkeys) by two delivery routes: intravenous (IV) injection and oral gavage (PO). Animals in both the IV and PO groups (e.g., n=3) are allowed free access to food and water, or may be fasted. A compound provided herein, or a pharmaceutically acceptable salt thereof, can be formulated in solution for the IV route and solution or suspension for the PO route. On the day of the experiment, the compound provided herein, or a pharmaceutically acceptable salt thereof, can be administered via vein injection (e.g., at 1 mg/kg) for IV route or via oral gavage (e.g., at 0.3 to 100 mg/kg) for PO route. Blood samples can be collected via serial bleeding (e.g., at 8 timepoints from 0.83 to 24 hours post dose). At each timepoint, blood can be collected (e.g., approximately 30 μL of blood/timepoint) in a K₂EDTA tube via a vein (e.g., the saphenous vein). Blood samples can be put on wet ice and centrifuged (e.g., at 4600 RPM for 4 minutes) to obtain plasma samples. Plasma samples can be diluted 1:1 (v/v) (e.g., with an equal volume of pH 3.0 phosphate buffer) and submitted to LC-MS/MS for sample analysis. Pharmacokinetic parameters, including clearance (IV), area under the curve (AUC), maximum concentration (Cₘₐₓ), the time at which maximum concentration is reached (Tₘₐₓ), mean residence time (MRT) and oral bioavailability (% F) can be calculated using a noncompartmental model.

In some embodiments, the pharmacokinetic parameters of a compound provided herein, or a pharmaceutically acceptable salt thereof, can be measured in a mouse model (e.g., a CD-1 (ICR) model). An exemplary protocol follows.

Mice (e.g., male CD-1 (ICR) mice, 7-9 weeks of age) are dosed with a compound provided herein, or a pharmaceutically acceptable salt thereof, once, either orally (PO) (e.g., at 3, 10, 30, 100, or 300 mg/kg) or intravenously (IV) (e.g., at 1 mg/kg). The compound provided herein, or a pharmaceutically acceptable salt thereof, is administered to the mice in a solution formulation (e.g., 100% PEG400 for the PO groups or 5% DMSO/10% Solutol/85% water for the IV group). The mice have access to food and water ad libitum.

Blood samples (e.g., 30 μL per sample) from the mice are taken at predetermined intervals, such as 5 minutes, 15 minutes, 30 minutes, 1 hour, 2 hours, 4 hours, 8 hours, 24 hours, 48 hours, and 72 hours) after the dose is administered (not every mouse will have every time point sampled, also called sparse sampling). The blood is sampled via saphenous vein puncture, then the blood samples (e.g., with K₂EDTA as anticoagulant) are temporarily put on ice and then centrifuged (e.g., at 4° C. and 4600 RPM for 5 minutes) within 30 minutes. Plasma samples are acidified via dilution with an equal volume of pH 3.0 phosphate buffer and put on dry ice. After the completion of the last sampling, all samples are stored at −80° C. or analyzed in a short time following collection. The concentration of the compound provided herein, or a pharmaceutically acceptable salt thereof, measured in an acidified plasma sample can be determined (e.g., via LC/MS/MS). For example, an acidified plasma sample is prepared for analysis using protein precipitation (e.g., by the addition of methanol), and an internal standard is spiked in at a known concentration. The spiked sample is mixed, centrifuged, and the supernatant is used in an LC/MS/MS method. The LC/MS/MS method uses a KINETEX® C18 2.6 μm 100 A (50 mm*2.10 mm) column with a first mobile phase of water (0.1% formic acid) and a second mobile phase of acetonitrile (0.1% formic acid). Multiple reaction monitoring is used to measure the analyte(s) of interest. Using the concentration of the compound provided herein, or a pharmaceutically acceptable salt thereof, in the plasma sample, pharmacokinetic parameters of terminal t_{1/2} (hr), tₘₐₓ (hr), Cₘₐₓ (ng/mL), AUCiₐₛt (hr*ng/mL), AUC_{Inf} (hr*ng/mL), AUC_{Extr} (%), MRT_{Inf} (hr), AUC_{Inf}/D (hr*kg*ng/mL/mg), and % F, are determined via noncompartmental analysis.

In some embodiments, the pharmacokinetic parameters of a compound provided herein, or a pharmaceutically acceptable salt thereof, can be measured in a rat model (e.g., a Sprague-Dawley rat model). An exemplary protocol follows.

Rats (e.g., male Sprague-Dawley rats, 7-9 weeks of age) are dosed with a compound provided herein, or a pharmaceutically acceptable salt thereof, either orally (PO) (e.g., at 10, 30, or 100 mg/kg) or intravenously (IV) (e.g., at 1 mg/kg). The compound provided herein, or a pharmaceutically acceptable salt thereof, is administered to the rats in a solution or suspension formulation (e.g., 10% 1-Methyl-2-Pyrrolidinone (NMP)/15% Solutol/75% (20% (2-Hydroxypropyl)-β-cyclodextrin (HPBCD) in water) for the PO group or 10% DMSO/10% Solutol/80% water solution for the IV group). The rats have access to food and water ad libitum.

Blood samples (e.g., 150 μL per sample) from the rats are taken at predetermined intervals, such as 5 minutes, 15, minutes, 30 minutes, 1 hour, 2 hours, 4 hours, 8 hours, 24 hours, 48 hours, and 72 hours after the dose is administered. The blood is sampled via jugular vein puncture, then the blood samples (e.g., with K₂EDTA as anticoagulant) are temporarily put on ice and then centrifuged (e.g., at 4° C. and 6000 RPM for 5 minutes) within 30 minutes. Plasma samples are acidified via dilution with an equal volume of pH 3.0 phosphate buffer and put on dry ice. After the completion of the last sampling, all samples are stored at −80° C. or analyzed in a short time following collection. The concentration of the compound provided herein, or a pharmaceutically acceptable salt thereof, can be measured in an acidified plasma sample (e.g., diluted 1:1 v/v with pH 3 phosphate buffer) can be determined (e.g., via LC/MS/MS). For example, an acidified plasma sample is prepared for analysis using protein precipitation (e.g., by the addition of methanol, then methanol/acetonitrile 1:1, v/v containing a known concentration of internal standard). The spiked sample is mixed, centrifuged, and the supernatant is used in an LC/MS/MS method. The LC/MS/MS method uses a KINETEX® C18 2.6 μm 100 A (50 mm*2.10 mm) column with a first mobile phase of water (0.1% formic acid (FA)) and a second mobile phase of acetonitrile (0.1% FA). Multiple reaction monitoring is used to measure the analyte(s) of interest. Using the concentration of the compound provided herein, or a pharmaceutically acceptable salt thereof, in the plasma sample, pharmacokinetic parameters of terminal t_{1/2} (hr), tₘₐₓ (hr), Cₘₐₓ (ng/mL), AUCₗₐₛₜ (hr*ng/mL), AUC_{Inf} (hr*ng/mL), AUC_{Extr} (%), MRT_{Inf} (hr), AUC_{Inf}/D (hr*kg*ng/mL/mg), % F, AUC₀₋₂₄ (hr*ng/mL), and AUC₄₈₋₇₂ (hr*ng/mL) are determined via noncompartmental analysis.

In some embodiments, the pharmacokinetic parameters of a compound provided herein, or a pharmaceutically acceptable salt thereof, can be measured in a dog model (e.g., a beagle model). An exemplary protocol follows.

Dogs (e.g., male Beagle dogs) are dosed with a compound provided herein, or a pharmaceutically acceptable salt thereof, orally (PO) (e.g., at 0.3, 1, 3 or 10 mg/kg) or intravenously (IV) (e.g., at 0.1 mg/kg). The compound provided herein, or a pharmaceutically acceptable salt thereof, is administered to the dogs in a solution or suspension formulation (e.g., 10% NMP/15% Solutol/75% (20% HPBCD in water)). The dogs are in the fed state.

Blood samples (e.g., about 0.5 mL) from the dogs are taken at predetermined intervals, such as 5 minutes, 15 minutes, 30 minutes, 1 hour, 2 hours, 4 hours, 8 hours, 24 hours, 48 hours, and 72 hours after the dose is administered. The blood is sampled via peripheral vein puncture, then the blood samples (e.g., with K₂EDTA as anticoagulant) are temporarily put on ice and then centrifuged (e.g., at 2-8° C. and 3200×g for 10 minutes) within 30 minutes. Plasma samples are acidified via dilution with an equal volume of pH 3.0 phosphate buffer and put on dry ice. After the completion of the last sampling, all samples are stored at −60 to −80° C. or analyzed in a short time following collection. The concentration of the compound provided herein, or a pharmaceutically acceptable salt thereof, can be measured in an acidified plasma sample can be determined (e.g., via LC/MS/MS). For example, an acidified plasma sample is prepared for analysis using protein precipitation (e.g., by the addition of acetonitrile containing internal standard of known concentration). The spiked sample is mixed, centrifuged, and the supernatant is used in an LC/MS/MS method. The LC/MS/MS method uses an ACQUITY UPLC HSS T3 1.8 μm 2.1×50 mm column (or an ACQUITY UPLC Protein BEH C4 300 Å 1.7 μm 2.1×50 mm column) with a first mobile phase of 0.1% formic acid and 2 mM ammonium formate in water/acetonitrile (v:v, 95:5) and a second mobile phase of 0.1% formic acid and 2 mM ammonium formate in acetonitrile/water (v:v, 95:5). Multiple reaction monitoring is used to measure the analyte(s) of interest. Using the concentration of the compound provided herein, or a pharmaceutically acceptable salt thereof, in the plasma sample, pharmacokinetic parameters of terminal t_{1/2} (hr), tₘₐₓ (hr), Cₘₐₓ (ng/mL), AUCₗₐₛₜ (hr*ng/mL), AUC_{Inf} (hr*ng/mL), AUC_{Extr} (%), MR_{Inf} (hr), % F, and AUC₀₋₂₄ (hr*ng/mL), are determined via noncompartmental analysis.

In some embodiments, the pharmacokinetic parameters of a compound provided herein, or a pharmaceutically acceptable salt thereof, can be measured in a non-human primate model (e.g., a cynomolgus monkey model). An exemplary protocol follows.

Non-human primates (e.g., male cynomolgus monkeys) are dosed with a compound provided herein, or a pharmaceutically acceptable salt thereof, orally (PO) (e.g., at 3 mg/kg) or intravenously (IV) (e.g., at 0.5 mg/kg). The compound provided herein, or a pharmaceutically acceptable salt thereof, is administered to the monkeys in a solution formulation (e.g., 10% NMP/15% Solutol/75% (20% HPBCD in water, pH 5)). The monkeys are in the fed state.

Blood samples (e.g., about 0.5 mL) from the monkeys are taken at predetermined intervals, such as 5 minutes, 15 minutes, 30 minutes, 1 hour, 2 hours, 4 hours, 8 hours, 24 hours, and 48 hours after the dose is administered. The blood is sampled via peripheral vein puncture, then the blood samples (e.g., with K₂EDTA as anticoagulant) are temporarily put on ice and then centrifuged (e.g., at 2-8° C. and 3200×g for 10 minutes) within 30 minutes. Plasma samples are acidified via dilution with an equal volume of pH 3.0 phosphate buffer and put on dry ice. After the completion of the last sampling, all samples are stored at −60 to −80° C. or analyzed in a short time following collection. The concentration of the compound provided herein, or a pharmaceutically acceptable salt thereof, can be measured in an acidified plasma sample via LC/MS/MS. For example, an acidified plasma sample is prepared for analysis using protein precipitation (e.g., by the addition of acetonitrile containing internal standard of known concentration). The spiked sample is mixed, centrifuged, and the supernatant is used in an LC/MS/MS method. The LC/MS/MS method uses an ACQUITY UPLC BEH C18 1.7 μm 2.1×50 mm column with a first mobile phase of 0.1% formic acid and 2 mM ammonium formate in water/acetonitrile (v:v, 95:5) and a second mobile phase of 0.1% formic acid and 2 mM ammonium formate in acetonitrile/water (v:v, 95:5). Multiple reaction monitoring is used to measure the analyte(s) of interest. Using the concentration of the compound provided herein, or a pharmaceutically acceptable salt thereof, in the plasma sample, pharmacokinetic parameters of terminal t_{1/2} (hr), tₘₐₓ (hr), Cₘₐₓ (ng/mL), AUCₗₐₛₜ (hr*ng/mL), AUC_{Inf} (hr*ng/mL), AUC_{Extr} (%), MRT_{Inf} (hr), % F, and AUC₀₋₂₄ (hr*ng/mL), are determined via noncompartmental analysis.

In some embodiments, the % F for a compound provided herein, or a pharmaceutically acceptable salt thereof, is at least 4%. In some embodiments, the % F for a compound provided herein, or a pharmaceutically acceptable salt thereof, is at least 10%. In some embodiments, the % F for a compound provided herein, or a pharmaceutically acceptable salt thereof, is at least 20%. In some embodiments, the % F for a compound provided herein, or a pharmaceutically acceptable salt thereof, is at least 30%. In some embodiments, the % F for a compound provided herein, or a pharmaceutically acceptable salt thereof, is at least 40%. In some embodiments, the % F for a compound provided herein, or a pharmaceutically acceptable salt thereof, is about 4% to about 80% (e.g., about 4% to about 60%, about 4% to about 40%, about 4% to about 20%, about 4% to about 10%, about 20% to about 40%, or about 20% to about 30%). In some embodiments, the % F for a compound provided herein, or a pharmaceutically acceptable salt thereof, is about 4% to about 20% (e.g., about 4% to about 10%). In some embodiments, the % F for a compound provided herein, or a pharmaceutically acceptable salt thereof, is about 20% to about 40%. In some embodiments, the % F for a compound provided herein, or a pharmaceutically acceptable salt thereof, is about 40% to about 60%. In some embodiments, the % F for a compound provided herein, or a pharmaceutically acceptable salt thereof, is about 60% to about 80%.

In some embodiments, the oral clearance (CL/F) for a compound provided herein, or a pharmaceutically acceptable salt thereof, dosed PO in rats at 10 mg/kg, is less than 10 mL/min/kg (e.g., less than 5 mL/min/kg, less than 3 mL/min/kg, or less than 1 mL/min/kg). In some embodiments, clearance for a compound provided herein, or a pharmaceutically acceptable salt thereof, dosed PO in rats at 10 mg/kg, is about 0.05 mL/min/kg to about 5 mL/min/kg (e.g., about 0.05 mL/min/kg to about 3 mL/min/kg, about 0.05 mL/min/kg to about 1 mL/min/kg, or about 0.05 mL/min/kg to about 0.5 mL/min/kg).

In some embodiments, the AUC for a compound provided herein, or a pharmaceutically acceptable salt thereof, dosed PO in rats at 10 mg/kg, is about 10 μM·h to about 350 μM·h (e.g., 10 μM·h to about 150 μM·h, about 50 μM·h to about 350 μM·h, about 100 μM·h to about 350 μM·h, or about 150 μM·h to about 350 μM·h).

In some embodiments, a compound provided herein, or a pharmaceutically acceptable salt thereof, is not a substrate of a human cytochrome P450 enzyme. In some embodiments, a compound provided herein, or a pharmaceutically acceptable salt thereof, is not a substrate of a human cytochrome P450 enzyme where ≥25% of clearance is attributed to that enzyme. In some embodiments, a compound provided herein, or a pharmaceutically acceptable salt thereof, is not an inhibitor and/or an inducer of one or more human cytochrome P450 enzymes.

In some embodiments, a compound provided herein, or a pharmaceutically acceptable salt thereof, is not an inhibitor and/or an inducer of one or more human cytochrome P450 enzymes, where the IC₅₀ value and/or EC₅₀ value for the one or more human cytochrome P450 enzymes, respectively, is significantly greater than the estimated free fraction concentration of a compound provided herein, or a pharmaceutically acceptable salt thereof, at a clinically relevant dose.

Exemplary human cytochrome P450 enzymes include those in the CYP1, CYP2, and CYP3 families. Of those, CYP1A1, CYP1A2, CYP1B1, CYP2A6, CYP2B6, CYP2C8, CYP2C9, CYP2C18, CYP2C19, CYP2D6, CYP2J2, CYP2S1, CYP2E1, CYP3A4, and CYP3A5 are known drug metabolizing enzymes. In some embodiments, no single cytochrome P450 enzyme is responsible for greater than or equal to 25% of the elimination of a compound provided herein, or a pharmaceutically acceptable salt thereof. Cytochrome P450 inhibition and/or inducing activity can be determined using appropriate in vitro assays, such as those described in the guidance document “In Vitro Drug Interaction Studies Cytochrome P450 Enzyme- and Transporter-Mediated Drug Interactions” provided by the U.S. F.D.A. in January 2020, and similarly in the ICH M12 guidance finalized in May 2024. For example, evaluation of cytochrome P450 inhibition can be performed in in vitro studies, in both a reversible and time-dependent manner. In an in vitro inhibition study, the ratio of intrinsic clearance values of a probe substrate for an enzymatic pathway in the absence and in the presence of a compound provided herein, or a pharmaceutically acceptable salt thereof, can be calculated based on these in vitro results; this ratio is referred to as R¹ for reversible inhibition, where R₁=1+(I_{max,u}/K_{i,u}), and I_{max,u} is the maximal unbound plasma concentration of the compound provided herein, or a pharmaceutically acceptable salt thereof, predicted in human, and K_{i,u} is the unbound inhibition constant determined in vitro. Specifically, in the case of CYP3A whereas the enzyme is also expressed at significant levels in the intestine, R_{1,gut} can be calculated where R_{1,gut}=1+(I_{gut}+K_{i,u}), and where I_{gut} is predicted human intestinal luminal concentration of the compound provided herein, or a pharmaceutically acceptable salt thereof, calculated as the human oral dose/250 mL. The time-dependent inhibition ratio R₂ can similarly be calculated, where R₂=(k_{obs}+k_{deg})/k_{deg}, and kobs is the observed (apparent first order) inactivation rate of the affected cytochrome P450 calculated by k_{obs}=(k_{inact}*I_{max,u})/(K_{I,u}+*I_{max,u}), k_{deg} is the apparent first-order degradation rate constant of the affected cytochrome P450, K_{I,u} is the unbound concentration of the compound provided herein, or a pharmaceutically acceptable salt thereof, causing half maximal inactivation, and k_{inact} is the maximal inactivation rate constant. Utilizing these equations for R₁, R_{1,gut}, and R₂, the compound provided herein, or a pharmaceutically acceptable salt thereof, can be evaluated for their potential to be an inhibitor of a cytochrome P450, and the drug-drug interaction (DDI) potential can be further investigated using mechanistic or physiological-based pharmacokinetic models of the liver and intestine and/or conducting a clinical DDI study with a sensitive index substrate. Additionally, a compound's propensity to activate nuclear receptors (e.g., PXR, CAR, or AhR) is evaluated through in vitro cytochrome P450 hepatocyte induction studies and the resulting data can be evaluated via the fold-change method, wherein the fold-change in cytochrome P450 enzyme mRNA levels, when incubated with the compound provided herein, or a pharmaceutically acceptable salt thereof, based on cutoff determined from known positive and negative controls to calibrate the system. For example, a compound provided herein, or a pharmaceutically acceptable salt thereof, is determined to be an inducer if: (1) it increases mRNA expression of a cytochrome P450 enzyme in a concentration-dependent manner; and (2) the fold change of cytochrome P450 mRNA expression relative to the vehicle control is ≥2-fold at the expected hepatic concentrations of the drug. Additionally, evaluation of cytochrome P450 induction can be performed by a correlation method, wherein correlation methods are used to predict the magnitude of a clinical induction effect (e.g., AUC ratio of an index substrate in the presence and absence of inducers) of a compound provided herein, or a pharmaceutically acceptable salt thereof, according to a calibration curve of relative induction scores (RIS) or I_{max,u}/EC₅₀ for a set of known inducers of the same cytochrome P450. If the predicted magnitude is more than a predefined cut-off (e.g., AUC ratio ≤0.8), a compound provided herein, or a pharmaceutically acceptable salt thereof, is considered to have induction potential in vivo.

In some embodiments, a compound provided herein, or a pharmaceutically acceptable salt thereof, can be tested for its potency in inhibiting hERG potassium channels. The cardiac potassium channel hERG is responsible for a rapid delayed rectifier current (I_{Kr}) in human ventricle, and inhibition of I_{Kr} is the most common cause of cardiac action potential prolongation by non-cardiac drugs. Increased action potential duration causes prolongation of the QT interval that has been associated with a dangerous ventricular arrhythmia, torsade de pointes. There are several methods of testing hERG inhibition potency, including Fastpatch hERG and manual patch clamp experiments.

In some embodiments, a compound provided herein, or a pharmaceutically acceptable salt thereof, is tested for its potency in inhibiting hERG potassium channels using a manual patch clamp assay. For example, in some embodiments, solutions or suspensions of a compound provided herein, or a pharmaceutically acceptable salt thereof, at several concentrations (e.g., 0.3, 1, 3 and 10 μM) can be exposed to single cells. The effect of the compound provided herein, or a pharmaceutically acceptable salt thereof, on the inhibition of hERG potassium channels can be measured in this system using an electrical pulse pattern, the data plotted, and an IC₅₀ value calculated for the inhibition of hERG by the compound provided herein, or a pharmaceutically acceptable salt thereof. An exemplary protocol follows.

In this experiment, hERG potassium channels are expressed in a human embryonic kidney cell line that lacks endogenous I_{Kr} (HEK-293 (ATCC 293T, CRL-3216™)). See, e.g., Brown, Arthur M., and David Rampe. Pharmaceutical News 7.4 (2000): 15-20; Weirich, Jorg, and H. Antoni, Basic Research in Cardiology 93 (1998): s125-s132, doi: 10.1007/s003950050236; and Yap, Yee Guan, and A. J. Camm. Clinical & Experimental Allergy 29 (1999): 174-181, doi: 10.1046/j.1365-2222.1999.0290s3174.x.

All chemicals used in solution preparations are purchased from a commercial supplier (e.g., Sigma-Aldrich) and are of ACS reagent grade purity or higher. Stock solutions of the compound provided herein, or a pharmaceutically acceptable salt thereof, positive control compound(s), and reference substance(s) are prepared in dimethyl sulfoxide (DMSO) and stored frozen. Solutions of each compound provided herein, or a pharmaceutically acceptable salt thereof, positive control compound, and reference substance concentrations are prepared fresh daily by diluting stock solutions into HEPES-buffered physiological saline solution (HB-PS; 137 mM NaCl, 4 mM KCl, 1.8 mM CaCl₂, 1 mM MgCl₂, 10 mM HEPES, and 10 mM glucose, pH 7.4). Since previous results have shown that ≤0.3% DMSO does not affect channel current, all test and control solutions can contain 0.3% DMSO.

In some embodiments, a positive control compound can be included in the experiment. In some such embodiments, the positive control compound can be terfenadine (Sigma-Aldrich), for example, in a HB-PS and 0.3% DMSO solution.

In some embodiments, a reference compound can be included in the experiment. In some such embodiments, the reference compound can be E-4031 (Sigma-Aldrich), for example, in a HB-PS and 0.3% DMSO solution.

If necessary, solutions are sonicated to facilitate dissolution. Visible precipitate observed during preparation or exposure of formulations to the test system is noted for reference.

The effect of a compound provided herein, or a pharmaceutically acceptable salt thereof, are initially evaluated at concentration range of 1 to 10 μM. Subsequent concentrations are evaluated based on the inhibition observed at that concentration.

HEK-293 cells are transfected with hERG cDNA. Stable transfectants are selected by coexpression of the G418-resistance gene incorporated into the expression plasmid. Selection pressure is maintained by including G418 in the culture medium. Cells are cultured in Dulbecco's Modified Eagle's Medium/Ham's Nutrient Mixture F-12 (DMEM/F-12) supplemented with 10% fetal bovine serum and the appropriate concentrations of penicillin G sodium, streptomycin sulfate, and G418.

All experiments are performed at near-physiological temperature (33-35° C.). Each cell acts as its own control.

The effect of a compound provided herein, or a pharmaceutically acceptable salt thereof, is evaluated at multiple (e.g., up to four) concentrations. Each concentration will be tested in at least three cells (n≥3). Additional concentration(s) may be tested due to the limits imposed by the physiochemical effects of the compound provided herein, or a pharmaceutically acceptable salt thereof (e.g., solubility or cytotoxicity).

The positive control compound is tested in at least two (2) cells (n≥2).

Cells are transferred to the recording chamber and superfused with vehicle control solution. Pipette (intracellular) solution for whole cell recordings is composed of 130 mM potassium aspartate, 5 mM MgCl₂, 5 mM EGTA, 4 mM ATP, and 10 mM HEPES, pH 7.2. Pipette solution is prepared in batches, aliquoted, stored frozen and a fresh aliquot thawed each day. The recording is performed at a temperature of 33 to 35° C. using a combination of in-line solution pre-heater, chamber heater, and feedback temperature controller. Temperature is measured using a thermistor probe in the recording chamber. Micropipettes for patch clamp recording are made from glass capillary tubing using a P-97 micropipette puller (Sutter Instruments). A commercial patch clamp amplifier (Molecular Devices) is used for whole patch clamp cell recordings. Before digitization, current records are low-pass filtered.

Cells stably expressing hERG are held at −80 mV. Onset and steady state inhibition of hERG potassium current due to the compound provided herein, or a pharmaceutically acceptable salt thereof, are measured using a pulse pattern with fixed amplitudes (conditioning prepulse of +20 mV for 1 second; repolarizing test ramp to −80 mV (at −0.5 V/second) repeated at 5 second intervals). Each recording ends with an application of a supramaximal concentration of the reference substance (e.g., E-4031 at 500 nM) to assess the contribution of endogenous currents. The remaining uninhibited current is subtracted off-line digitally from the data to determine the potency of the compound provided herein, or a pharmaceutically acceptable salt thereof, for hERG inhibition. Peak current is measured during the test ramp. A steady state is maintained for at least 20 seconds before applying the compound provided herein, or a pharmaceutically acceptable salt thereof, positive control compound, or reference substance. Peak tail currents are measured until a new steady state is achieved. If a steady state cannot be reached within 12 minutes, the response at 12 minutes is substituted for the steady state value and a notation made.

Data acquisition and analyses are performed using the commercial suite of pCLAMP programs (Molecular Devices). Steady state is defined by the limiting constant rate of change with time (linear time dependence). The steady state before and after application of each compound provided herein, or a pharmaceutically acceptable salt thereof, is used to calculate the percentage of current inhibited at each concentration.

In some embodiments, a compound provided herein, or a pharmaceutically acceptable salt thereof, is tested for its potency in inhibiting hERG potassium channels (e.g., encoded by the KCNH₂ gene and expressed in HEK293 cells) using a Fastpatch hERG assay. For example, in some embodiments, solutions or suspensions of a compound provided herein, or a pharmaceutically acceptable salt thereof, at several concentrations (e.g., 0.3, 1, 3 and 10 μM) can be exposed to cells in an automatic parallel patch clamp system. The effect of the compound provided herein, or a pharmaceutically acceptable salt thereof, on the inhibition of hERG potassium channels can be measured in this system using an electrical pulse pattern, the data plotted, and an IC₅₀ value calculated for the inhibition of hERG by the compound provided herein, or a pharmaceutically acceptable salt thereof. An exemplary protocol follows.

The in vitro effects of a compound provided herein, or a pharmaceutically acceptable salt thereof, are evaluated at room temperature using the QPATCH HT® (Sophion Bioscience A/S, Denmark), an automatic parallel patch clamp system. The compound provided herein, or a pharmaceutically acceptable salt thereof, is exposed to hERG at 0.3, 1, 3 and 10 μM in at least five cells (n≥5). The duration of exposure to each compound concentration is at least three minutes.

Solutions or suspensions of the compound(s) provided herein, or a pharmaceutically acceptable salt thereof, are prepared daily. Various concentrations are prepared by diluting stock solutions into an appropriate HEPES-buffered physiological saline solution (TB-PS).

Previous results have shown that 0.3% DMSO does not affect channel current. Therefore, all test and control solutions can contain up to 0.3% DMSO.

Each formulation of a compound provided herein, or a pharmaceutically acceptable salt thereof, can be sonicated (e.g., Model 2510/5510, Branson Ultrasonics, Danbury, CT), e.g., at room temperature, to facilitate dissolution.

In some embodiments, a positive control can be included in the experiment. In some such embodiments, the positive control compound can be cisapride (Tocris Bioscience), for example, in a 0.3% DMSO solution.

In preparation for the recording session, a glass-lined 96-well compound plate is loaded with the appropriate amounts of solutions of compound(s) provided herein, or a pharmaceutically acceptable salt thereof, and control solutions, and placed in the plate well of the QPATCH® (Sophion Bioscience A/S, Denmark).

HEK293 (ATCC 293T, CRL-3216™) cells are stably transfected with the appropriate ion channel cDNA(s). Except for cells that have been stored frozen, stable transfectants are maintained in the culture medium with the appropriate selection pressure and antibiotics.

All experiments are performed at room temperature. Each cell acts as its own control.

Vehicle is applied via the QPATCH® robot pipetting system to naive cells for a 5-10 minute exposure interval. After vehicle application, various concentrations of a compound provided herein, or a pharmaceutically acceptable salt thereof, are applied in at least three (3) minute intervals (n~3, where n=the number of cells/concentration). Each solution exchange on the QPATCH® is performed multiple times, which results in 100% replacement of the compound in the QPlate.

Positive control(s) are applied in the same manner as the compound provided herein, or a pharmaceutically acceptable salt thereof, to verify sensitivity to ion channel blockade.

In preparation for a recording session, an intracellular solution (e.g., 137 mM NaCl, 4 mM KCl, 1.8 mM CaCl₂, 1 mM MgCl₂, 10 mM HEPES, and 10 mM glucose, pH 7.4) is loaded into the intracellular compartments of the QPlate and cell suspension is pipetted into the extracellular compartments. After establishment of a whole-cell configuration, membrane currents are recorded using up to 48 parallel patch clamp amplifiers in the QPATCH® system.

Valid whole-cell recordings meet the following criteria:
1. Membrane resistance ≥200 MΩ.
2. Leak current ≤25% channel current or subtracted.

Onset and block of hERG current is measured using a stimulus voltage pattern consisting of a 500 ms prepulse to −40 mV (leakage subtraction), a 2-second activating pulse to +40 mV followed by a 2-second test pulse to −40 mV. The pulse pattern is repeated continuously at 10 s intervals from a holding potential of −80 mV. Peak tail current is measured during the −40 mV test pulse. Leakage current is calculated from the current amplitude evoked by the −40 mV prepulse and subtracted from the total membrane current record.

Data acquisition and analyses are performed using conventional software. Steady state is defined by the limiting constant rate of change with time (linear time dependence). The steady state before and after application of a compound provided herein, or a pharmaceutically acceptable salt thereof, will be used to calculate the percentage of current inhibited at each concentration. If current is blocked ≥50%, estimated IC₅₀ values will be calculated using the following formula:

$$\% Inhibition = \{ 1 - 1 / [ 1 + {( [ Test ] / {IC}_{50} )}^{N} ] \} * 100$$

Where [Test] is the concentration of the compound provided herein, or a pharmaceutically acceptable salt thereof, IC₅₀ is the concentration of the compound provided herein, or a pharmaceutically acceptable salt thereof, at half-maximal inhibition, N is the Hill coefficient, and % Inhibition is the percentage of current inhibited at each test article concentration. Nonlinear least squares fits are solved with the Solver add-in for Excel (Microsoft, WA). Overestimation of current inhibition, due to rundown, is avoided by a manual adjustment that uses an exponential compensation formula in the QPATCH® analysis software.

In some embodiments, a compound provided herein, or a pharmaceutically acceptable salt thereof, is not a hERG inhibitor. In some embodiments, a compound provided herein, or a pharmaceutically acceptable salt thereof, inhibits hERG with an IC₅₀ of greater than 60 nM (e.g., greater than 100 nM, 300 nM, 500 nM, 1 μM, 3 μM, 5 μM, 10 μM, 20 μM, or 30 μM). For example, a compound provided herein, or a pharmaceutically acceptable salt thereof, inhibits hERG with an IC₅₀ of greater than 500 nM (e.g., greater than 1 μM, 3 μM, 5 μM, 10 μM, 20 μM, or 30 μM). In some embodiments, a compound provided herein, or a pharmaceutically acceptable salt thereof, inhibits hERG with an IC₅₀ of greater than 1 μM (e.g., greater than 3 μM, 5 μM, 10 μM, 20 μM, or 30 μM). In some embodiments, a compound provided herein, or a pharmaceutically acceptable salt thereof, inhibits hERG with an IC₅₀ of greater than 10 μM (e.g., greater than 20 μM or 30 μM). In some embodiments, a compound provided herein, or a pharmaceutically acceptable salt thereof, inhibits hERG with an IC₅₀ of greater than 30 μM.

In some embodiments, a compound provided herein, or a pharmaceutically acceptable salt thereof, can be evaluated for potential secondary pharmacology. For example, in some embodiments, solutions of a compound provided herein, or a pharmaceutically acceptable salt thereof, at a single or several concentrations can be exposed to a panel of primary cells, cell lines or isolated tissues expressing known pharmacological receptors, enzymes or transporters. The effect of the compound provided herein, or a pharmaceutically acceptable salt thereof, the secondary pharmacological target of interest can be determined, the data is initially plotted as a percent inhibition. For secondary targets exceeding an inhibition threshold (typically ≥50%), an IC₅₀ value is generated and calculated following an 8-point concentration curve of the compound provided herein, or a pharmaceutically acceptable salt thereof.

An exemplary list of assays is included in Table 1 below. One or more of these assays can be used to assess the secondary pharmacological characteristics of a compound provided herein. Exemplary protocols are described in Valentin, Jean-Pierre, and Tim Hammond. Journal of Pharmacological and Toxicological Methods 58.2 (2008): 77-87; doi: 10.1016/j.vascn.2008.05.007; Wakefield, Ian D., et al. Fundamental & Clinical Pharmacology 16.3 (2002): 209-218, doi: 10.1046/j.1472-8206.2002.00099.x; Whitebread, Steven, et al. Drug Discovery Today 10.21 (2005): 1421-1433, doi: 10.1016/S1359-6446(05)03632-9; and Lounkine, Eugen, et al. Nature 486.7403 (2012): 361-367, doi: 10.1038/naturel 1159.

TABLE 1

| Protein | Organism | Assay Type | Family |
|---|---|---|---|
| Acetylcholinesterase | Human | Enzymatic | Neurotransmitter |
| | | | Metabolism |
| Angiotensin- | Human | Enzymatic | Protease |
| converting | | | |
| peptidase | | | |
| ATPase (Na/K) | Pig | Enzymatic | Transporter |
| Cathespin G | Human | Enzymatic | Protease |
| chymotrypsin peptidase | | | |
| Cyclooxygenase 1 | Human | Enzymatic | Lipid Metabolism |
| Cyclooxygenase 2 | Human | Enzymatic | Lipid Metabolism |
| Monoamine Oxidase A | Human | Enzymatic | Oxireductase |
| Monoamine Oxidase B | Human | Enzymatic | Oxireductase |
| Phosphodiesterase 3A | Human | Enzymatic | PDE |
| Phosphodiesterase 4D2 | Human | Enzymatic | PDE |
| Insulin Receptor Kinase | Human | Enzymatic | Kinase |
| lrc Kinase | Human | Enzymatic | Kinase |
| PKC alpha Kinase | Human | Enzymatic | Kinase |
| Adenosine A1 Receptor | Human | Binding | GPCR |
| Adenosine Transporter | Human | Binding | Transporter |
| Alpha 1A Adrenergic | Human | Binding | GPCR |
| Receptor | | | |
| Alpha 1B Adrenergic | Human | Binding | GPCR |
| Receptor | | | |
| Alpha 1D Adrenergic | Human | Binding | GPCR |
| Receptor | | | |
| Alpha 2A Adrenergic | Human | Binding | GPCR |
| Receptor | | | |
| Alpha 2B Adrenergic | Human | Binding | GPCR |
| Receptor | | | |
| Beta 1 Adrenergic | Human | Binding | GPCR |
| Receptor | | | |
| Beta 2 Adrenergic | Human | Binding | GPCR |
| Receptor | | | |
| Androgen Receptor | Human | Binding | Nuclear Hormone |
| | | | Receptor |
| Angiotensin 1 Receptor | Human | Binding | GPCR |
| Bradyknin 2 Receptor | Human | Binding | GPCR |
| Cav1.2 (L-type Diltizem) | Rat | Binding | Ion Channel |
| Calcium Channel | | | |
| Cav1.2 (L-type | Rat | Binding | Ion Channel |
| Dihydropyridine) | | | |
| Calcium Channel | | | |
| Cav1.2 (L-type | Rat | Binding | Ion Channel |
| Verpamil) Calcium | | | |
| Channel | | | |
| Cav2.2 (N-typel) | Rat | Binding | Ion Channel |
| Calcium Channel | | | |
| Cannabinoid 1 | Human | Binding | GPCR |
| Cannabinoid 2 | Human | Binding | GPCR |
| Chemokine Receptor 1 | Human | Binding | GPCR |
| Cholecystokinin 1 | Human | Binding | GPCR |
| Receptor | | | |
| Cholecystokinin 2 | Human | Binding | GPCR |
| Receptor | | | |
| Dopamine 1 Receptor | Human | Binding | GPCR |
| Dopamine 2L Receptor | Human | Binding | GPCR |
| Dopamine 2L Receptor | Human | Binding | GPCR |
| Dopamine Transporter | Human | Binding | Transporter |
| Endothelial Receptor A | Human | Binding | GPCR |
| Estrogen Receptor Alpha | Human | Binding | Nuclear Hormone |
| | | | Receptor |
| GABA (nonselective) | Rat | Binding | Transporter |
| Transporter | | | |
| GABA A (nonselective) | Rat | Binding | Ion Channel |
| Flunitrazepam | | | |
| GABA A (nonselective) | Rat | Binding | Ion Channel |
| Ro-15-1788 | | | |
| GABA A (nonselective) | Rat | Binding | Ion Channel |
| TBOB | | | |
| GABA B Receptor | Human | Binding | GCPR |
| B1a/B2 | | | |
| Glucocorticoid Receptor | Human | Binding | Nuclear Hormone |
| | | | Receptor |
| Glutamate AMPA | Rat | Binding | Ion Channel |
| (nonselective) | | | |
| Glutamate Kainate | Rat | Binding | Ion Channel |
| (nonselective) | | | |
| Glutamate NMDA | Rat | Binding | Ion Channel |
| (nonselective) MDL | | | |
| 105,519 | | | |
| Glutamate NMDA | Rat | Binding | Ion Channel |
| (nonselective) TCP | | | |
| Glutamate NMDA | Rat | Binding | Ion Channel |
| (nonselective) Ifenprodil | | | |
| Metabotropic glutatmate | Human | Binding | GPCR |
| Receptor 5 | | | |
| Glycine (nonselective) | Rat | Binding | Ion Channel |
| Strychine | | | |
| Histamine Receptor 1 | Human | Binding | GPCR |
| Histamine Receptor 2 | Human | Binding | GPCR |
| CXC Receptor 2 | Human | Binding | GPCR |
| Cys Leukotriene | Human | Binding | GPCR |
| Receptor 1 | | | |
| Melanocortin Receptor 1 | Human | Binding | GPCR |
| Melanocortin Receptor 4 | Human | Binding | GPCR |
| Muscarinic Receptor 1 | Human | Binding | GPCR |
| Muscarinic Receptor 2 | Human | Binding | GPCR |
| Muscarinic Receptor 3 | Human | Binding | GPCR |
| Muscarinic Receptor 4 | Human | Binding | GPCR |
| Neurokinin 1 Receptor | Human | Binding | GPCR |
| Neuropeptide Y Receptor | Human | Binding | GPCR |
| N Acetylcholine | Human | Binding | Ion Channel |
| Receptor Alpha 1 | | | |
| N Acetylcholine | Human | Binding | Ion Channel |
| Receptor Alpha 3/Beta4 | | | |
| Delta Opioid Receptor | Human | Binding | GPCR |
| Kappa Opioid Receptor | Human | Binding | GPCR |
| Mu-Opioid Receptor | Human | Binding | GPCR |
| Potassium ATP Receptor | Hamster | Binding | Ion Channel |
| hERG Potassium | Human | Binding | Ion Channel |
| Channel Dofetilide | | | |
| Peroxisomes proliferator | Human | Binding | Nuclear Hormone |
| Receptor (agonist) | | | Receptor |
| Serotonin 1A Receptor | Human | Binding | GPCR |
| (Antagonist) | | | |
| Serotonin 2A Receptor | Human | Binding | GPCR |
| (Agonist) | | | |
| Serotonin 2B Receptor | Human | Binding | GPCR |
| (Antagonist) | | | |
| Serotonin 2C Receptor | Human | Binding | GPCR |
| (Antagonist) | | | |
| Serotonin 3 Receptor | Human | Binding | Ion Channel |
| (Antagonist) | | | |
| Serotonin Transporter | Human | Binding | Transporter |
| Sodium (Nav1.5) | Human | Binding | Ion Channel |
| Channel Site 2 | | | |
| Vasopressin 1A Receptor | Human | Binding | GPCR |
| Oxytocin | | | |
| Progesterone Receptor | Human | Binding | Nuclear Hormone |
| | | | Receptor |
| Platelet Activating Factor | Human | Binding | GPCR |
| (agonist) | | | |

Heterobifunctional degraders can, in some cases, induce the degradation of off-target proteins. For heterobifunctional degraders that utilize CRBN, common off-target proteins that can be degraded include GSPT1, IKZF1, TKZF2, TKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1α is a kinase that is involved in many key cellular processes including cell cycle progression and chromosome segregation; these are both commonly essential genes, so undesired degradation of either or both may lead to nonspecific cytotoxicity. The TKZF proteins are zinc finger transcription factors that are involved with cell fate during hematopoiesis, and degradation of these proteins has been associated with hematotoxicity. See, e.g., Moreau, Kevin, et al. British Journal ofPharmacology 177.8 (2020): 1709-1718, doi: 10.1111/bph.15014.

In some embodiments, the compounds provided herein, or pharmaceutically acceptable salts thereof, can exhibit potent and selective induction of degradation of a BCL-X_{L} protein. In some embodiments, a compound provided herein, or a pharmaceutically acceptable salt thereof, or a pharmaceutically acceptable salt thereof, can selectively target a BCL-X_{L} protein for degradation over an off-target protein, such as another BCL-2 family member (e.g., BCL-2 and/or MCL-1) or non-BCL-2 family member target (e.g., GSPT1, IKZF1, IKZF2, IKZF3, and/or CK1α).

As used herein, “selective” or “selectively”, when referring to a compound provided herein, or a pharmaceutically acceptable salt thereof in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay. In some embodiments, the specified protein is BCL-X_{L} protein and the comparator is BCL-2 protein. For example, if a compound provided herein, or a pharmaceutically acceptable salt thereof, “selectively” induces degradation of BCL-X_{L} protein over BCL-2 protein as determined by a degradation assay, then the compound has at least a 5-fold (e.g., at least a 10-fold, at least a 25-fold, at least a 50-fold, or at least a 100-fold) smaller DC₅₀ value for BCL-X_{L} protein than for the BCL-2 protein when measured by the degradation assay.

In some embodiments, the compounds provided herein can exhibit potency (e.g., nanomolar potency) against a BCL-X_{L} protein with minimal activity (e.g., micromolar potency) against BCL-2 family members (e.g., BCL-2 or MCL-1 proteins). In some embodiments, a compound provided herein, or a pharmaceutically acceptable salt thereof, can exhibit potent degradation of a BCL-X_{L} protein and have minimal potency in degrading (e.g., as measured by Yₘᵢₙ, DC₅₀, EC₅₀, and/or Dₘₐₓ values) an off-target protein (e.g., a BCL-2 family member (e.g., BCL-2 and/or MCL-1), GSPT1, IKZF1, IKZF2, IKZF3 and/or CK1α).

In some embodiments, a compound provided herein, or a pharmaceutically acceptable salt thereof, can exhibit greater induction of degradation of a BCL-X_{L} protein relative to induction of degradation (e.g., as measured by Yₘᵢₙ, DC₅₀, EC₅₀, and/or Dₘₐₓ values) of an off-target protein (e.g., a BCL-2 family member (e.g., BCL-2 and/or MCL-1), GSPT1, IKZF1, IKZF2, IKZF3, and/or CK1α). In some embodiments, a compound provided herein, or a pharmaceutically acceptable salt thereof, can exhibit at least 2-fold, 3-fold, 5-fold, 10-fold, 25-fold, 50-fold, or 100-fold greater induction of degradation of a BCL-X_{L} protein relative to induction of degradation of an off-target protein. In some embodiments, a compound provided herein, or a pharmaceutically acceptable salt thereof, can exhibit up to 1000-fold greater induction of degradation of a BCL-X_{L} protein relative to induction of degradation of an off-target protein. In some embodiments, a compound provided herein, or a pharmaceutically acceptable salt thereof, can exhibit from about 2-fold to about 10-fold greater induction of degradation of a BCL-X_{L} protein relative to induction of degradation of an off-target protein (e.g., GSPT1, IKZF1, IKZF2, and/or IKZF3 and/or CK1α) (e.g., as measured by Yₘᵢₙ, DC₅₀, EC₅₀, and/or Dₘₐₓ values). In some embodiments, a compound provided herein, or a pharmaceutically acceptable salt thereof, can exhibit from about 10-fold to about 100-fold greater induction of degradation of a BCL-X_{L} protein relative to induction of degradation of an off-target protein. In some embodiments, a compound provided herein, or a pharmaceutically acceptable salt thereof, can exhibit from about 100-fold to about 1000-fold greater induction of degradation of a BCL-X_{L} protein relative to induction of degradation of an off-target protein.

In some embodiments, a compound provided herein, or a pharmaceutically acceptable salt thereof, can exhibit from about 1000-fold to about 10000-fold greater induction of degradation of a BCL-X_{L} protein relative to induction of degradation of an off-target protein.

Certain agents that inhibit or induce degradation of BCL-X_{L} have demonstrated platelet toxicity, which resulted in dose-limiting toxicity (e.g., thrombocytopenia) in the clinic. See, e.g., Adams and Cory, Cell Death & Differentiation 25.1 (2018): 27-36, doi: 10.1038/cdd.2017.161; Campbell and Tait. Open Biology 8.5 (2018): 180002, doi: 10.1098/rsob.180002; Pullarkat et al., Cancer Discovery (2021), doi:10.1158/2159-8290.CD-20-1465; Negi and Voisin-Chiret. ChemBioChem (2022), doi: 10.1002/cbic.202100689. The viability of platelets may be monitored with any appropriate assay, such as those described herein. See, for instance, Example B4.

In some embodiments, a therapeutically effective amount of a compound provided herein, or a pharmaceutically acceptable salt thereof, results in at least about 30% platelet viability (e.g., at least about 50% platelet viability, or at least about 80% platelet viability) when administered to a subject (e.g., compared to the platelet count of the subject prior to administration of the compound provided herein, or a pharmaceutically acceptable salt thereof).

In some embodiments, a therapeutically effective amount of a compound provided herein, or a pharmaceutically acceptable salt thereof, results in about 30% to about 100% platelet viability (e.g., about 50% to about 100% platelet viability, or about 80% to about 100% platelet viability) when administered to a subject (e.g., compared to the platelet count of the subject prior to administration of the compound provided herein, or a pharmaceutically acceptable salt thereof).

In some embodiments, a therapeutically effective amount of a compound provided herein, or a pharmaceutically acceptable salt thereof, results in at least about 30% platelet viability (e.g., at least about 50% platelet viability, or at least about 80% platelet viability) when administered to a subject (e.g., compared to the platelet count of the subject prior to administration of the compound provided herein, or a pharmaceutically acceptable salt thereof) and has a Yₘᵢₙ value of about 50% to about 70% for a BCL-X_{L} protein. In some embodiments, a therapeutically effective amount of a compound provided herein, or a pharmaceutically acceptable salt thereof, results in at least about 30% platelet viability (e.g., at least about 50% platelet viability, or at least about 80% platelet viability) when administered to a subject (e.g., compared to the platelet count of the subject prior to administration of the compound provided herein, or a pharmaceutically acceptable salt thereof) and has a Yₘᵢₙ value of less than about 50% for a BCL-X_{L} protein. In some embodiments, a therapeutically effective amount of a compound provided herein, or a pharmaceutically acceptable salt thereof, results in at least about 30% platelet viability (e.g., at least about 50% platelet viability, or at least about 80% platelet viability) when administered to a subject (e.g., compared to the platelet count of the subject prior to administration of the compound provided herein, or a pharmaceutically acceptable salt thereof) and has a Yₘᵢₙ value of about 0% to about 50% for a BCL-X_{L} protein.

In some embodiments, a therapeutically effective amount of a compound provided herein, or a pharmaceutically acceptable salt thereof, results in between about 30% and 100% platelet viability (e.g., about 50% to about 100% platelet viability, or about 80% to about 100% platelet viability) when administered to a subject (e.g., compared to the platelet count of the subject prior to administration of the compound provided herein, or a pharmaceutically acceptable salt thereof) and has a Yₘᵢₙ value of about 50% to about 70% for a BCL-X_{L} protein.

In some embodiments, a therapeutically effective amount of a compound provided herein, or a pharmaceutically acceptable salt thereof, results in between about 30% and 100% platelet viability (e.g., about 50% to about 100% platelet viability, or about 80% to about 100% platelet viability) when administered to a subject (e.g., compared to the platelet count of the subject prior to administration of the compound provided herein, or a pharmaceutically acceptable salt thereof) and has a Yₘᵢₙ value of less than about 50% for a BCL-X_{L} protein. In some embodiments, a therapeutically effective amount of a compound provided herein, or a pharmaceutically acceptable salt thereof, results in between about 30% and 100% platelet viability (e.g., about 50% to about 100% platelet viability, or about 80% to about 100% platelet viability) when administered to a subject (e.g., compared to the platelet count of the subject prior to administration of the compound provided herein, or a pharmaceutically acceptable salt thereof) and has a Yₘᵢₙ value of about 0% to about 50% for a BCL-X_{L} protein.

In some embodiments, therapeutically effective amount of a compound provided herein, or a pharmaceutically acceptable salt thereof, results in at least about 50% platelet viability (e.g., at least about 80% platelet viability) when administered to a subject (e.g., compared to the platelet count of the subject prior to administration of the compound provided herein, or a pharmaceutically acceptable salt thereof) and has a Yₘᵢₙ value of about 50% to about 70% for a BCL-X_{L} protein. In some embodiments, a therapeutically effective amount of a compound provided herein, or a pharmaceutically acceptable salt thereof, results in at least about 50% platelet viability (e.g., at least about 80% platelet viability) when administered to a subject (e.g., compared to the platelet count of the subject prior to administration of the compound provided herein, or a pharmaceutically acceptable salt thereof) and has a Yₘᵢₙ value of less than about 50% for a BCL-X_{L} protein. In some embodiments, a therapeutically effective amount of a compound provided herein, or a pharmaceutically acceptable salt thereof, results in at least about 50% platelet viability (e.g., at least about 80% platelet viability) when administered to a subject (e.g., compared to the platelet count of the subject prior to administration of the compound provided herein, or a pharmaceutically acceptable salt thereof) and has a Yₘᵢₙ value of about 0% to about 50% for a BCL-X_{L} protein.

In some embodiments, a therapeutically effective amount of a compound provided herein, or a pharmaceutically acceptable salt thereof, results in between about 50% and 100% platelet viability (e.g., about 80% to about 100% platelet viability) when administered to a subject (e.g., compared to the platelet count of the subject prior to administration of the compound provided herein, or a pharmaceutically acceptable salt thereof) and has a Yₘᵢₙ value of about 50% to about 70% for a BCL-X_{L} protein. In some embodiments, a therapeutically effective amount of a compound provided herein, or a pharmaceutically acceptable salt thereof, results in between about 50% and 100% platelet viability (e.g., about 80% to about 100% platelet viability) when administered to a subject (e.g., compared to the platelet count of the subject prior to administration of the compound provided herein, or a pharmaceutically acceptable salt thereof) and has a Yₘᵢₙ value of less than about 50% for a BCL-X_{L} protein. In some embodiments, a therapeutically effective amount of a compound provided herein, or a pharmaceutically acceptable salt thereof, results in between about 50% and 100% platelet viability (e.g., about 80% to about 100% platelet viability) when administered to a subject (e.g., compared to the platelet count of the subject prior to administration of the compound provided herein, or a pharmaceutically acceptable salt thereof) and has a Yₘᵢₙ value of about 0% to about 50% for a BCL-X_{L} protein.

In some embodiments, a compound provided herein, or a pharmaceutically acceptable salt thereof, when tested at a concentration of about 0.25 μM to about 3 μM in the assay in Example B4, shows at least about 30% platelet viability (e.g., at least about 50% platelet viability, or at least about 80% platelet viability). In some embodiments, a compound provided herein, or a pharmaceutically acceptable salt thereof, when tested at a concentration of about 0.25 μM to about 3 μM in the assay in Example B4, shows about 30% to about 100% platelet viability (e.g., about 50% to about 100% platelet viability, or about 80% to about 100% platelet viability).

In some embodiments, a compound provided herein, or a pharmaceutically acceptable salt thereof, when tested at a concentration of about 0.25 μM to about 3 μM in the assay in Example B4, shows at least about 30% platelet viability (e.g., at least about 50% platelet viability, or at least about 80% platelet viability) and has a Yₘᵢₙ value of about 50% to about 70% in the assay described in Example B1. In some embodiments, a compound provided herein, or a pharmaceutically acceptable salt thereof, when tested at a concentration of about 0.25 μM to about 3 μM in the assay in Example B4, shows at least about 30% platelet viability (e.g., at least about 50% platelet viability, or at least about 80% platelet viability) and has a Yₘᵢₙ value of less than about 50% in the assay described in Example B1. In some embodiments, a compound provided herein, or a pharmaceutically acceptable salt thereof, when tested at a concentration of about 0.25 μM to about 3 μM in the assay in Example B4, shows at least about 30% platelet viability (e.g., at least about 50% platelet viability, or at least about 80% platelet viability) and has a Yₘᵢₙ value of about 0% to about 50% in the assay described in Example B1.

In some embodiments, a compound provided herein, or a pharmaceutically acceptable salt thereof, when tested at a concentration of about 0.25 μM to about 3 μM in the assay in Example B4, shows between about 30% and 100% platelet viability (e.g., about 50% to about 100% platelet viability, or about 80% to about 100% platelet viability) and has a Yₘᵢₙ value of about 50% to about 70% in the assay described in Example B1. In some embodiments, a compound provided herein, or a pharmaceutically acceptable salt thereof, when tested at a concentration of about 0.25 μM to about 3 μM in the assay in Example B4, shows between about 30% and 100% platelet viability (e.g., about 50% to about 100% platelet viability, or about 80% to about 100% platelet viability) and has a Yₘᵢₙ value of less than about 50% in the assay described in Example B1. In some embodiments, a compound provided herein, or a pharmaceutically acceptable salt thereof, when tested at a concentration of about 0.25 μM to about 3 μM in the assay in Example B4, shows between about 30% and 100% platelet viability (e.g., about 50% to about 100% platelet viability, or about 80% to about 100% platelet viability) and has a Yₘᵢₙ value of about 0% to about 50% in the assay described in Example B1.

In some embodiments, a compound provided herein, or a pharmaceutically acceptable salt thereof, when tested at a concentration of about 0.25 μM to about 3 μM in the assay in Example B4, shows at least about 50% platelet viability (e.g., at least about 80% platelet viability) and has a Yₘᵢₙ value of about 50% to about 70% in the assay described in Example B1. In some embodiments, a compound provided herein, or a pharmaceutically acceptable salt thereof, when tested at a concentration of about 0.25 μM to about 3 μM in the assay in Example B4, shows at least about 50% platelet viability (e.g., at least about 80% platelet viability) and has a Yₘᵢₙ value of less than about 50% in the assay described in Example B1. In some embodiments, a compound provided herein, or a pharmaceutically acceptable salt thereof, when tested at a concentration of about 0.25 μM to about 3 μM in the assay in Example B4, shows at least about 50% platelet viability (e.g., at least about 80% platelet viability) and has a Yₘᵢₙ value of about 0% to about 50% in the assay described in Example B1.

In some embodiments, a compound provided herein, or a pharmaceutically acceptable salt thereof, when tested at a concentration of about 0.25 μM to about 3 μM in the assay in Example B4, shows between about 50% and 100% platelet viability (e.g., about 80% to about 100% platelet viability) and has a Yₘᵢₙ value of about 50% to about 70% in the assay described in Example B1. In some embodiments, a compound provided herein, or a pharmaceutically acceptable salt thereof, when tested at a concentration of about 0.25 μM to about 3 μM in the assay in Example B4, shows between about 50% and 100% platelet viability (e.g., about 80% to about 100% platelet viability) and has a Yₘᵢₙ value of less than about 50% in the assay described in Example B1. In some embodiments, a compound provided herein, or a pharmaceutically acceptable salt thereof, when tested at a concentration of about 0.25 μM to about 3 μM in the assay in Example B4, shows between about 50% and 100% platelet viability (e.g., about 80% to about 100% platelet viability) and has a Yₘᵢₙ value of about 0% to about 50% in the assay described in Example B1.

Provided herein is a method of treating a cancer in a subject in need of such treatment, the method comprising administering to the subject a therapeutically effective amount of a compound provided herein, or a pharmaceutically acceptable salt thereof, or a pharmaceutical composition thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound provided herein, or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is a method of treating a cancer in a subject in need of such treatment, the method comprising:
(a) detecting a biomarker (e.g., a mutation, an amplification, a copy number increase, and/or expression (optionally including level of expression) of the biomarker) associated with the cancer; and
(b) administering to the subject a therapeutically effective amount of a compound provided herein, or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound provided herein) to have a biomarker (e.g., a mutation, an amplification, a copy number increase, and/or expression (optionally including level of expression) of the biomarker) associated with the cancer, the method comprising administering to the subject a therapeutically effective amount of a compound provided herein, or a pharmaceutically acceptable salt thereof, or a pharmaceutical composition thereof, as a monotherapy or in combination with an additional therapy or therapeutic agent.

Provided herein is a method of treating a cancer in a subject in need of such treatment, the method comprising:
(a) detecting a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer; and
(b) administering to the subject a therapeutically effective amount of a compound provided herein, or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound provided herein) to have a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer, the method comprising administering to the subject a therapeutically effective amount of a compound provided herein, or a pharmaceutically acceptable salt thereof, or a pharmaceutical composition thereof, as a monotherapy or in combination with an additional therapy or therapeutic agent.

Provided herein is use of a compound provided herein, or a pharmaceutically acceptable salt thereof, or a pharmaceutical composition thereof, for the treatment of cancer, for example, any of the cancers provided herein.

Provided herein is a compound provided herein, or a pharmaceutically acceptable salt thereof, or a pharmaceutical composition thereof, for use in the treatment of cancer, for example, any of the cancers provided herein.

Provided herein is use of a compound provided herein, or a pharmaceutically acceptable salt thereof, or a pharmaceutical composition thereof, as a medicament for the treatment of cancer, for example, any of the cancers provided herein.

Provided herein is use of a compound provided herein, or a pharmaceutically acceptable salt thereof, in the manufacture of a medicament for the treatment of cancer, for example, any of the cancers provided herein.

Provided herein is a compound provided herein, or a pharmaceutically acceptable salt thereof, or a pharmaceutical composition thereof, for use as a medicament. Also provided herein is a compound provided herein, or a pharmaceutically acceptable salt thereof, or a pharmaceutical composition thereof, for use as a medicament for the treatment of cancer, for example, any of the cancers provided herein.

Provided herein is a compound provided herein, or a pharmaceutically acceptable salt thereof, or a pharmaceutical composition thereof, for use in treating a cancer, for example, any of the cancers provided herein.

As used herein, “monotherapy”, when referring to a compound provided herein, or a pharmaceutically acceptable salt thereof, means that the compound provided herein, or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments (sometimes also called supportive care measures) for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, thrombocytopenia, neutropenia, anemia, or gastrointestinal symptoms, including nausea, diarrhea, and lack of appetite.

For example, in some embodiments, the supportive care measure can be granulocyte colony stimulating factor (G-CSF), a thrombopoietin receptor (TpoR) agonist, or a steroid.

In some embodiments, the TpoR agonist is avatrombopag (e.g., avatrombopag maleate), eltrombopag (e.g., eltrombopag olamine), lusutrombopag, megapoietin (KRN-9000), pegacaristim, rafutrombopag (e.g., rafutrombopag olamine), recombinant human thrombopoietin, romiplostim, totrombopag (e.g., totrombopag choline), or a combination thereof.

As used herein, “the subject has previously received one or more therapeutic agents or therapies for the cancer” means that the subject has been previously administered one or more therapeutic agents or therapies (e.g., anticancer agent or therapy) for the cancer other than a compound provided herein, or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1, the Lugano 2014 criteria (Cheson, Bruce D., et al. Journal of Clinical Oncology 32.27 (2014): 3059-3067; doi: 10.1200/JCO.2013.54.8800) or the Global Response Criteria (Olsen, Elise A., et al. Blood, The Journal of the American Society of Hematology 140.5 (2022): 419-437; doi: 10.1182/blood.2021012057)). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, neuroblastoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma (e.g., intrahepatic cholangiocarcinoma)), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor (GIST), liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic eosinophilic leukemia (CEL), chronic lymphocytic leukemia (CLL), chronic neutrophilic leukemia (CNL), acute lymphocytic leukemia (ALL) (e.g., Philadelphia chromosome positive ALL or T cell ALL (I-ALL)), acute myeloid leukemia (AML) (e.g., acute promyelocytic leukemia (APL), post-MPN AML, post-myelodysplastic syndrome (post-MDS) AML, M6-AML (also known as pure erythroid leukemia (PEL)), or M7-AML (also known as acute megakaryoblastic leukemia (AKML))), chronic myeloid leukemia (CML)), lymphoma (e.g., Hodgkin lymphoma (e.g., nodular lymphocyte predominant Hodgkin lymphoma (NLPHL)), or non-Hodgkin lymphoma (e.g., Burkitt lymphoma (BL), diffuse large B cell lymphoma (DLBCL), diffuse histiocytic lymphoma (DHL), follicular lymphoma (FL), intravascular large B cell lymphoma (IVLBCL), mantle cell lymphoma (MCL), small lymphocytic lymphoma (SLL), a T cell lymphoma (e.g., anaplastic large T cell lymphoma, cutaneous T cell lymphoma, or peripheral T cell lymphoma))), essential thrombocythemia, polycythemia vera, myelofibrosis (e.g., primary myelofibrosis, post-essential thrombocythemia myelofibrosis, or post-polycythemia vera myelofibrosis), a myelodysplastic syndrome (MDS) (e.g., M6 MDS or M7 MDS), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, malignant pleural mesothelioma, non-small cell lung cancer (NSCLC), small cell lung cancer), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma or Merkel cell carcinoma), thymus cancer (e.g., thymoma), or a combination thereof.

In some embodiments, the cancer is breast cancer, gastrointestinal cancer (e.g., bile duct cancer (e.g., cholangiocarcinoma (e.g., intrahepatic cholangiocarcinoma)), colorectal cancer (CRC), gastrointestinal stromal tumor, or pancreatic cancer), genitourinary cancer (e.g., bladder cancer (e.g., bladder urothelial carcinoma) or kidney cancer), gynecologic cancer (e.g., cervical cancer, ovarian cancer (e.g., high grade serous ovarian cancer (HGSOC), low grade serous ovarian cancer (LGSOC)), or uterine cancer), head and neck cancer (e.g., head and neck squamous cell carcinoma), hematological cancer (e.g., leukemia (e.g., acute lymphocytic leukemia (ALL) (e.g., T-ALL), acute myeloid leukemia (AML) (e.g., APL, post-MPN AML, post-MDS AML, M6-AML, or M7-AML), chronic lymphocytic leukemia (CLL)), lymphoma (e.g., follicular lymphoma (FL), small lymphocytic lymphoma (SLL), a T cell lymphoma (e.g., anaplastic large T cell lymphoma, cutaneous T cell lymphoma, or peripheral T cell lymphoma), or diffuse large B cell lymphoma (DLBCL)), essential thrombocythemia, polycythemia vera, myelofibrosis (e.g., primary myelofibrosis, post-essential thrombocythemia myelofibrosis, or post-polycythemia vera myelofibrosis), or a myelodysplastic syndrome (MDS) (e.g., M6 MDS or M7 MDS)), lung cancer (e.g., non small cell lung cancer (NSCLC), small cell lung cancer (SCLC), lung squamous cell carcinoma, or malignant pleural mesothelioma (MPM)), neuroblastoma, sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), or skin cancer (e.g., melanoma or Merkel cell carcinoma).

In some embodiments, the cancer is a myeloproliferative neoplasm (MPN). In some embodiments, the myeloproliferative neoplasm is CEL, CML, CNL, essential thrombocythemia, polycythemia vera, or myelofibrosis (e.g., primary myelofibrosis, post-essential thrombocythemia myelofibrosis, or post-polycythemia vera myelofibrosis). In some embodiments, the MPN has a JAK2 mutation (e.g., a JAK2 V617F mutation). In some embodiments, the MPN does not have a JAK2 mutation. In some embodiments, the cancer is low-risk myelofibrosis. In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis). In some embodiments, the cancer is intermediate or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation). In some embodiments, the cancer is intermediate or high-risk myelofibrosis (e.g., primary myelofibrosis, post-essential thrombocythemia myelofibrosis, or post-polycythemia vera myelofibrosis) without a JAK2 V617F mutation. In some embodiments, the subject has a platelet count below 50×10⁹/L.

In some embodiments, the cancer is polycythemia vera. In some embodiments, the cancer is polycythemia vera, and the subject has had an inadequate response to or is intolerant of hydroxyurea.

In some embodiments, the cancer is an MDS. In some embodiments, the cancer is M6 MDS. In some embodiments, the cancer is M7 MDS.

In some embodiments, the cancer is T-ALL. In some embodiments, the cancer is relapsed/refractory T-ALL.

In some embodiments, the cancer is CRC (e.g., Braf mutant CRC (e.g., Braf V600E CRC) or KRas mutant CRC (e.g., KRas G12C CRC or KRas G12D CRC)).

In some embodiments, the cancer is SCLC (e.g., ASCL1 subtype SCLC orNEUROD1 subtype SCLC).

In some embodiments, the cancer is NSCLC (e.g., Braf mutant NSCLC (e.g., Braf V600E NSCLC), EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), KRas mutant NSCLC (e.g., KRas G12C NSCLC)).

In some embodiments, the cancer is lung squamous cell carcinoma.

In some embodiments, the cancer is malignant pleural mesothelioma (e.g., BAP1 mutant malignant pleural mesothelioma). In some embodiments, the cancer is malignant pleural mesothelioma, and a compound provided herein, or a pharmaceutically acceptable salt thereof, is administered as a monotherapy.

In some embodiments, the cancer is melanoma (e.g., Braf mutant melanoma (e.g., Braf V600E melanoma)).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), HER2 low breast cancer, triple negative breast cancer, hormone receptor positive breast cancer (ER+ and/or PR+, with or without HER2 positivity)).

In some embodiments, the cancer is lymphoma. In some embodiments, the lymphoma is a T cell lymphoma (e.g., anaplastic large T cell lymphoma, cutaneous T cell lymphoma, or peripheral T cell lymphoma). In some embodiments, the lymphoma is a non-Hodgkin lymphoma (e.g., DLBCL, anaplastic large T cell lymphoma, cutaneous T cell lymphoma, or peripheral T cell lymphoma). In some embodiments, the lymphoma is peripheral T cell lymphoma.

In some embodiments, the cancer is leukemia. In some embodiments, the leukemia is a T cell leukemia (e.g., T cell ALL). In some embodiments, the cancer is post-MPN leukemia.

In some embodiments, the cancer is a T cell leukemia (e.g., T cell ALL), and a compound provided herein, or a pharmaceutically acceptable salt thereof, is administered as a monotherapy.

In some embodiments, the cancer is M6-AML. In some embodiments, the M6-AML is a post-MPN AML. In some embodiments, the M6-AML is a post-myelodysplastic syndrome (MDS) AML. In some embodiments, the cancer is M6-AML, and the compound provided herein, or pharmaceutically acceptable salt thereof, is administered as a monotherapy.

In some embodiments, the cancer is M7-AML. In some embodiments, the M7-AML is a post-MPN AIL. In some embodiments, the M7-AML is a post-MDS AML. In some embodiments, the cancer is M7-AML, and the compound provided herein, or pharmaceutically acceptable salt thereof, is administered as a monotherapy.

In some embodiments, the cancer is head and neck cancer.

In some embodiments, the cancer is essential thrombocythemia.

In some embodiments, the cancer is polycythemia vera.

In some embodiments, the cancer is myelofibrosis. In some embodiments, the cancer is primary myelofibrosis. In some embodiments, the cancer is post-essential thrombocythemia myelofibrosis. In some embodiments, the cancer is post-polycythemia vera myelofibrosis.

In some embodiments, the cancer is an MDS. In some embodiments, the MDS is M6 MDS. IN some embodiments, the MDS is M7 MDS. In some embodiments, the cancer is an MDS, and a compound provided herein, or a pharmaceutically acceptable salt thereof, is administered as a monotherapy.

In some embodiments, the cancer is pancreatic cancer.

In some embodiments, the cancer is bladder cancer (e.g., bladder urothelial carcinoma).

In some embodiments, the cancer is ovarian cancer (e.g., BRCA1 mutant ovarian cancer or BRCA2 mutant ovarian cancer). In some embodiments, the cancer is HGSOC (e.g., BRCA1 mutant HGSOC or BRCA2 mutant HGSOC).

In some embodiments, the cancer is cervical cancer.

In some embodiments, the cancer is colorectal cancer.

In some embodiments, the cancer is skin cancer. In some embodiments, the skin cancer is melanoma. In some embodiments, the cancer is Merkel cell carcinoma. In some embodiments, the cancer is Merkel cell carcinoma, and a compound provided herein, or a pharmaceutically acceptable salt thereof, is administered as a monotherapy.

In some embodiments, the cancer is neuroblastoma.

In some embodiments, the cancer is intrahepatic cholangiocarcinoma.

In some embodiments, the cancer is a mesenchymal cancer. In some embodiments, the mesenchymal cancer is mesenchymal breast cancer or mesenchymal kidney cancer.

In some embodiments, a BCL-X_{L} copy number gain or a BCL-X_{L} amplification can be detected in a sample from the subject (e.g., detecting three or more copies of a BCL2L1 gene in the sample from the subject). In some embodiments, the subject was determined (e.g., prior to administration of a compound provided herein) to have a cancer having a BCL-X_{L} copy number gain or a BCL-X_{L} amplification.

In some embodiments, the cancer has a BCL-X_{L} copy number gain.

In some embodiments, the cancer has a BCL-X_{L} amplification.

Non-limiting examples of BCL-X_{L} involvement in cancers can be found in: Wilson, Wyndham H., et al. The Lancet Oncology 11.12 (2010): 1149-1159, doi: 10.1016/S1470-2045(10)70261-8; Keitel, Ulrike, et al. Oncotarget 5.23 (2014): 11778, doi: 10.18632/oncotarget.2634; Chonghaile, Triona Ni, et al. Cancer Discovery 4.9 (2014): 1074-1087, doi: 10.1158/2159-8290.CD-14-0353; Zaanan, Aziz, et al. Journal of Biological Chemistry 290.39 (2015): 23838-23849, doi: 10.1074/jbc.M115.657833; Zhang, Haichao, et al. Molecular Cancer 14.1 (2015): 1-9, doi: 10.1186/s12943-015-0397-y; Soderquist, R^{Ya} n S., et al. Nature Communications 9.1 (2018): 1-13, doi: 10.1038/s41467-018-05815-z; Stover, Elizabeth H., et al. Molecular Cancer Research 17.11 (2019): 2281-2293, doi: 10.1158/1541-7786.MCR-18-1243; Concoran, R. B., et al. Annals of Oncology (2019) 30 (suppl_5): v164, doi: 10.1093/annonc/mdz244.009; Lakhani, Nehal J., et al. Journal of Clinical Oncology (2020): 3509-3509, doi: 10.1200/JCO.2020.38; He, Yonghan, et al. Journal ofHematology & Oncology 13.1 (2020): 1-13, doi: 10.1186/s13045-020-00928-9; Grubb, Treg, et al. Clinical Cancer Research (2022), doi: 10.1158/1078-0432.CCR-22-0669; Joly, Florence, et al. Gynecologic Oncology 165.1 (2022): 30-39, doi: 10.1016/j.ygyno.2022.01.021; and Nanjo, Shigeki, et al. The Journal of ClinicalInvestigation (2022), doi: 10.1172/JCI145099.

In some embodiments, the subject has previously been treated with another anticancer agent, a chemotherapeutic agent, radiation, surgery, a multi-kinase inhibitor, or a combination thereof.

Provided herein is a method of treating an ocular disease or condition in a subject in need of such treatment, the method comprising administering (e.g., intravitreally or topically) to the subject a therapeutically effective amount of a compound provided herein, or a pharmaceutically acceptable salt thereof, or a pharmaceutical composition thereof. In some embodiments, the ocular disease or condition is diabetic macular edema. In some embodiments, the ocular disease or condition is age-related macular degeneration. In some embodiments, the ocular disease or condition is diabetic retinopathy. See, e.g., Crespo-Garcia, Sergio, et al. Cell Metabolism 33.4 (2021): 818-832, doi: 10.1016/j.cmet.20211.011; Hassan, Jannah Waled, and Ashay D. Bhatwadekar, Frontiers in Pharmacology 13 (2022): 896907, doi: 10.3389/fphar.2022.896907.

Provided herein is use of a compound provided herein, or a pharmaceutically acceptable salt thereof, or a pharmaceutical composition thereof, for the treatment of an ocular disease or condition, for example, any of the ocular diseases or conditions provided herein.

Provided herein is use of a compound provided herein, or a pharmaceutically acceptable salt thereof, or a pharmaceutical composition thereof, as a medicament for the treatment of an ocular disease or condition, for example, any of the ocular diseases or conditions provided herein.

Provided herein is use of a compound provided herein, or a pharmaceutically acceptable salt thereof, in the manufacture of a medicament for the treatment of an ocular disease or condition, for example, any of the ocular diseases or conditions provided herein.

Also provided herein is a compound provided herein, or a pharmaceutically acceptable salt thereof, or a pharmaceutical composition thereof, for use as a medicament for the treatment of an ocular disease or condition, for example, any of the ocular diseases or conditions provided herein.

Provided herein is a compound provided herein, or a pharmaceutically acceptable salt thereof, or a pharmaceutical composition thereof, for use in treating an ocular disease or condition, for example, any of the ocular diseases or conditions provided herein.

Also provided herein is a method of treating a fibrotic disease or condition and/or a disease or condition associated with senescent cells in a subject in need of such treatment, the method comprising administering to the subject a therapeutically effective amount of a compound provided herein, or a pharmaceutically acceptable salt thereof, or a pharmaceutical composition thereof. Non-limiting examples of fibrotic diseases or conditions and/or diseases or conditions associated with senescent cells include pulmonary fibrosis (e.g., idiopathic pulmonary fibrosis, systemic fibrosis-associated lung disease, radiation-associated pulmonary fibrosis), radiation-associated skin fibrosis, liver fibrosis, primary sclerosing cholangitis, diabetic macular edema, age-related macular degeneration, diabetic retinopathy, geographic atrophy, ischemia and reperfusion injury, heart failure, recovery from acute myocardial infarction, pulmonary hypertension, inflammatory bowel disease, colitis, Crohn's disease, diabetes, aging skin (including photoaging-related pigmentation), donor organ transplant survival and function, stem cell transplant survival and function, osteoarthritis, recovery from spinal cord injury, Alzheimer's disease, tau-opathies, progressive supranuclear palsy, and age-related neurological decline (e.g., related to impaired neurovascular coupling). See, e.g., Zhu, Y. I., et al. Aging Cell 14.4 (2015): 644-658, doi: 10.1111/ace1.12344; Zhu, Y. I., et al. Aging Cell 15.3 (2016): 428-435, doi: 10.1111/ace1.12445; Chang, Jianhui, et al. Nature Medicine 22.1 (2016): 78-83, doi: 10.1038/nrn.4010; Zhu, Yi, et al. Aging (Albany NY) 9.3 (2017): 955, doi: 10.18632/aging.101202; Lagares, David, et al. Science Translational Medicine 9.420 (2017): eaa13765, doi: 10.1126/scitranslmed.aa13765; Pan, Jin, et al. International Journal of Radiation Oncology Biolog Physics 99.2 (2017): 353-361, doi: 10,1016/j.ijrobp.2017.02 216; Bussian, Tyler J., et al. Nature 562.7728 (2018): 578-582, doi: 10.1038/s41586-018-0543-y; Moncsek, Anja, et al. Hepatology 67.1 (2018): 247-259, doi: 10.1002/hep.29464; van Willigenburg, Hester, Peter L J de Keizer, and Ron W F de Bruin. Pharmacological Research 130 (2018): 322-330, doi: 10.1016/j.phrs.2018.02.015; Walaszczyk, Anna, et al. Aging Cell 18.3 (2019): e12945, doi: 10.1111/ace1.12945; Aguayo-Mazzucato, Cristina, et al. Cell Metabolism 30.1 (2019): 129-142, doi: 10.1016/j.cmet.2019.05.006; Sessions, Garrett A., et al. The FASEB Journal 133.11 (2019): 12364, doi: 10 1096/fj.201900815RR; Gerdes, Erin O. Wissler, et al. International Review of Neurobiology 155 (2020): 203-234, doi: 10.1016/bs.irn.2020.03.019; Sasaki, Motoko, Yasunori Sato, and Yasuni Nakanuma. Journal of Autoimmunity 107 (2020): 102377. doi: 10.1016/j.jaut.2019.102377; Yabluchanskiy, Andriy, et al. Genoscience 42. (2020): 409-428, doi: 10.1007/s11357-020-00154-8; Dookun, Emily, et al. Aging Cell 19.10 (2020): e13249, doi: 10.1111/ace1.13249; Jia, Kangni, et al Journal of Cardiovascular Pharmacology 76.4 (2020): 452-460, doi: 10.1097/FJC.0000000000000878; Sierra-Ramirez, Arantzazu, et al. Aging (Albany NY) 12.12 (2020): 11337, doi: 10,18632/aging.103607; Yang, Hlao, et al. Aging (Albany NY) 12.13 (2020): 12750, doi: 10.18632/aging. 103177; Lawrie, Allan, and Sheila E. Francis. The Journal of Clinical Investigation 131.11 (2021): e149721, doi: 10.1172/JC1149721; Paramos-de-Carvalho, Diogo, et al. Cell Reports 36.1 (2021): 109334, doi: 10.1016/j.celrep.2021.109334; Tarantini, Stefano, et al GeroScience 43.5 (2021): 2427-2440. doi: 10.1007/s11357-021-00440-z; Park, Ji Hee, et alt The British Journal of Dermatology (2022) 186(4):740-742, doi: 10. bd20893; Fielder, Edward, et al. ELife 11 (2022): e75492, doi: 10.7554/eLife.75492; Suzuki, Keiji, et al. Mutation Research Genetic Toxicology and Environmental Mutagenesis 876 (2022): 503448, doi: 10.1016/j.mrgentox.2022.503448; He, An, et al. American Journal of Transplantation 22.11 (2022): 2529-2547, doi: 10.1111/ajt.17154; Johnson, Laura A., et al. Inflammatory Bowel Diseases 28.2 (2022): 161-175, doi: 10.1093/ibd/izab166; Miura, Yugo, et al. Stem Cell Research & Therapy 13.1 (2022): 222, doi: 10.1186/s13287-022-02901-4; Cooley, Joseph C., et a. JCI Insight 8.3 (2023): e163762, doi: 10.1172/jci.insight.163762; Watanabe, Yusuke, et al. Hepatology Research 53 (2023): 460-472, doi: 10.1111/hepr.13879; and Takaya, Kento, et al., Rejuvenation Research 26.1 (2023): 9-20, doi: 10.1089/rej.2022.0048.

Provided herein is use of a compound provided herein, or a pharmaceutically acceptable salt thereof, or a pharmaceutical composition thereof, for the treatment of a fibrotic disease or condition and/or a disease or condition associated with senescent cells, for example, any of the fibrotic diseases or conditions and/or diseases or conditions associated with senescent cells provided herein.

Provided herein is use of a compound provided herein, or a pharmaceutically acceptable salt thereof, or a pharmaceutical composition thereof, as a medicament for the treatment of a fibrotic disease or condition and/or a disease or condition associated with senescent cells, for example, any of the fibrotic diseases or conditions and/or diseases or conditions associated with senescent cells provided herein.

Provided herein is use of a compound provided herein, or a pharmaceutically acceptable salt thereof, in the manufacture of a medicament for the treatment of a fibrotic disease or condition and/or a disease or condition associated with senescent cells, for example, any of the fibrotic diseases or conditions and/or diseases or conditions associated with senescent cells provided herein.

Also provided herein is a compound provided herein, or a pharmaceutically acceptable salt thereof, or a pharmaceutical composition thereof, for use as a medicament for the treatment of a fibrotic disease or condition and/or a disease or condition associated with senescent cells, for example, any of the fibrotic diseases or conditions and/or diseases or conditions associated with senescent cells provided herein.

Provided herein is a compound provided herein, or a pharmaceutically acceptable salt thereof, or a pharmaceutical composition thereof, for use in treating a fibrotic disease or condition and/or a disease or condition associated with senescent cells, for example, any of the fibrotic diseases or conditions and/or diseases or conditions associated with senescent cells provided herein.

Also provided is a method for modulating (e.g., decreasing) BCL-X_{L} protein activity in a cell, comprising contacting the cell with an effective compound provided herein, or a pharmaceutically acceptable salt thereof. In some embodiments, the contacting is in vitro. In some embodiments, the contacting is in vivo. In some embodiments, the contacting is in vivo, wherein the method comprises administering an effective amount of a compound provided herein, or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein.

As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL-X_{L} protein in a cell, comprising contacting the cell with a compound provided herein, or a pharmaceutically acceptable salt thereof. In some embodiments, the level of BCL-X_{L} protein is decreased by at least 30% (e.g., at least 40%, at least 50%, at least 60%, at least 70%, at least 80%, at least 90%, at least 95%, at least 97%, or at least 99%) compared to a cell not contacted with the compound provided herein, or a pharmaceutically acceptable salt thereof. In some embodiments, the contacting is in vitro. In some embodiments, the contacting is in vivo. In some embodiments, the contacting is in vivo, wherein the method comprises administering an effective amount of a compound provided herein, or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL-X_{L} protein in a cell, comprising contacting the cell with a compound provided herein, or a pharmaceutically acceptable salt thereof. In some embodiments, the contacting is in vitro. In some embodiments, the contacting is in vivo. In some embodiments, the contacting is in vivo, wherein the method comprises administering an effective amount of a compound provided herein, or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL-X_{L} protein, a compound provided herein, or a pharmaceutically acceptable salt thereof, and a CRBN protein or fragment thereof in a cell, comprising contacting the cell with a compound provided herein, or a pharmaceutically acceptable salt thereof. In some embodiments, the contacting is in vitro. In some embodiments, the contacting is in vivo. In some embodiments, the contacting is in vivo, wherein the method comprises administering an effective amount of a compound provided herein, or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method of inhibiting cell proliferation, in vitro or in vivo, the method comprising contacting a cell with an effective amount of a compound provided herein, or a pharmaceutically acceptable salt thereof, or a pharmaceutical composition thereof as defined herein.

Further provided herein is a method of increasing cell death, in vitro or in vivo, the method comprising contacting a cell with an effective amount of a compound provided herein, or a pharmaceutically acceptable salt thereof, or a pharmaceutical composition thereof as defined herein. Also provided herein is a method of increasing tumor cell death in a subject. The method comprises administering to the subject a compound provided herein, or a pharmaceutically acceptable salt thereof, in an amount effective to increase tumor cell death.

When employed as pharmaceuticals, the compounds provided herein, or pharmaceutically acceptable salts thereof, can be administered in the form of pharmaceutical compositions as described herein.

Also provided herein is a method for inducing degradation of a BCL-X_{L} protein in a mammalian cell, the method comprising contacting the mammalian cell with an effective amount of a compound provided herein, or a pharmaceutically acceptable salt thereof.

Also provided herein is a method of treating a subject having a cancer, wherein the method comprises:
administering a therapeutically effective amount of a compound provided herein, or a pharmaceutically acceptable salt thereof, as a monotherapy or in conjunction with a first anticancer agent to the subject who has been administered one or more doses of the first anticancer agent to the subject.

Also provided herein is a method of treating a subject having a cancer, wherein the method comprises:
(a) administering one or more doses of a first anticancer agent to the subject; and
(b) after (a), administering a therapeutically effective amount of a compound provided herein, or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject; and
(b) after (a), administering a therapeutically effective amount of a compound provided herein, or a pharmaceutically acceptable salt thereof, as a monotherapy or in conjunction with a second anticancer agent to the subject.

### Combinations

In any of the indications described herein, a compound provided herein, or a pharmaceutically acceptable salt thereof, can be used as a monotherapy. In some embodiments, a compound provided herein, or a pharmaceutically acceptable salt thereof, can be used prior to administration of an additional therapeutic agent or additional therapy. For example, a subject in need thereof can be administered one or more doses of a compound provided herein, or a pharmaceutically acceptable salt thereof, for a period of time and then undergo at least partial resection of the tumor. In some embodiments, the treatment with one or more doses of a compound provided herein, or a pharmaceutically acceptable salt thereof, reduces the size of the tumor (e.g., the tumor burden) prior to the at least partial resection of the tumor.

In some embodiments, a subject in need thereof can be administered one or more doses of a compound provided herein, or a pharmaceutically acceptable salt thereof, for a period of time and after one or more rounds of radiation therapy. In some embodiments, the treatment with one or more doses of a compound provided herein, or a pharmaceutically acceptable salt thereof, reduces the size of the tumor (e.g., the tumor burden) prior to the one or more rounds of radiation therapy.

In some embodiments of any the methods described herein, the compound provided herein, or a pharmaceutically acceptable salt thereof, is administered in combination with a therapeutically effective amount of at least one additional therapeutic agent selected from one or more additional therapies or therapeutic (e.g., chemotherapeutic) agents.

Non-limiting examples of additional therapies and therapeutic agents include: RAS pathway targeted therapeutic agents (e.g., Ras/RAF/MEK/PI3K pathway inhibitors, (e.g., Ras inhibitors (e.g., KRas inhibitors), KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, Abl inhibitors (e.g., BCR-Abl inhibitors), ALK inhibitors, AKT inhibitors, AURKA inhibitors, mTOR inhibitors, CDK2 inhibitors, CDK4/5 inhibitors, CDK4/6 inhibitors, CDK7 inhibitors, CDK9 inhibitors, MET (also known as cMET) inhibitors, FAK inhibitors, FGFR1 inhibitors, FGFR2 inhibitors, FGFR3 inhibitors, FGFR4 inhibitors, ErbB family inhibitors (e.g., EGFR inhibitors, anti-EGFR antibodies or anti-EGFR antibody-drug conjugates, HER2 inhibitors, anti-HER2 antibodies or anti-HER2 antibody-utilizing biologics), JAK inhibitors (e.g., JAK inhibitors having activity against JAK1, JAK2, and/or JAK1/2), Src inhibitors, VEGFR inhibitors), LSD1 inhibitors, EZH2 inhibitors, BET inhibitors, STING agonists, telomerase inhibitors, mTORC1 inhibitors, YAP inhibitors, proteasome inhibitors, farnesyl transferase inhibitors, Hif2a inhibitors, HSP90 inhibitors, PTEN inhibitors, PARP inhibitors, signal transduction pathway inhibitors, checkpoint inhibitors, modulators of the apoptosis pathway (e.g., BCL-2 inhibitors), chemotherapeutics, angiogenesis-targeted therapies, immune-targeted agents, including immunomodulatory imide drugs (sometimes called “IMiDs” or “CELMoDs”) and immunotherapy (e.g., anti-PD1 therapy or anti-PD-L1 therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapy or therapeutic agent is an Abl inhibitor (e.g., a BCR-Abl inhibitor), an ALK inhibitor, an AURKA inhibitor, a BCL-2 inhibitor, a Braf inhibitor, a CDK2 inhibitor, a CDK4/6 inhibitor, a CDK7 inhibitor, a CDK9 inhibitor, an EGFR inhibitor, an anti-EGFR antibody or anti-EGFR antibody-drug conjugate, an ERK inhibitor, an EZH2 inhibitor, a FGFR1 inhibitor, a FGFR2 inhibitor, a FGFR3 inhibitor, a FGFR4 inhibitor, a HER2 inhibitor, an anti-HER2 antibody or anti-HER2 antibody-drug conjugate, a JAK inhibitor, a KRas inhibitor, a MEK inhibitor, a MET inhibitor, a Hif2a inhibitor, a PARP inhibitor, a VEGFR inhibitor, an LSD1 inhibitor, a BET inhibitor, a STING agonist, a telomerase inhibitor, a TORC1/2 inhibitor, an immunomodulatory imide drug, immunotherapy (e.g., a PD-1 inhibitor (e.g., anti-PD1 therapy), a PD-L1 inhibitor (e.g., anti-PD-L1 therapy)), L-asparaginase, chemotherapy, radiotherapy, or a combination thereof.

In some embodiments, the additional therapy or therapeutic agent is an Abl degrader (e.g., a BCR-Abl degrader), an ALK degrader, an AURKA degrader, a BCL-2 degrader, a BRaf degrader, a CDK2 degrader, a CDK4/6 degrader, a CDK7 degrader, a CDK9 degrader, an EGFR degrader, an ERK degrader, an EZH2 degrader, a FGFR1 degrader, a FGFR2 degrader, a FGFR3 degrader, a FGFR4 degrader, a HER2 degrader, a JAK2 degrader, a KRas degrader, a MEK degrader, a MET degrader, a Hif2a degrader, a PARP degrader, a VEGFR degrader, an LSD1 degrader, a BET degrader, a telomerase degrader, a TORC1/2 degrader, an immunomodulatory imide drug, immunotherapy (e.g., anti-PDi therapy or anti-PD-L1 therapy), chemotherapy, radiotherapy, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IkT-148009, NPB-001-056, or a combination thereof.

In some embodiments, the ALK inhibitor is alectinib (e.g., alectinib hydrochloride), brigatinib, ceritinib, crizotinib, ensartinib (e.g., ensartinib hydrochloride), entrectinib, fidrisertib, lorlatinib, TQ-B-3101, TQ-B-3139, or a combination thereof. In some embodiments, the ALK inhibitor is alectinib (e.g., alectinib hydrochloride), brigatinib, ceritinib, crizotinib, ensartinib (e.g., ensartinib hydrochloride), fidrisertib, lorlatinib, TQ-B-3101, TQ-B-3139, or a combination thereof.

In some embodiments, the AURKA inhibitor is alisertib, danusertib, ilorasertib, tinengotinib, AT-9283, BI-811283, ENMD-2076, or a combination thereof.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), ZN-d5, or a combination thereof.

In some embodiments, the cancer is a lung cancer (e.g., SCLC), and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a lung cancer (e.g., SCLC), and the additional therapy or therapeutic agent is venetoclax.

In some embodiments, the cancer is a non-Hodgkin lymphoma, and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a non-Hodgkin lymphoma, and the additional therapy or therapeutic agent is venetoclax.

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C₁₇₀₇₁₄₇₉—F, CHIR-265 (RAF265), FORE-8394 (PLX-8394), GDC-0879, GDC-5573 (HM95573), HLX-208, PLX-3603, PLX-4720, or a combination thereof.

In some embodiments, a BRaf V600E mutation can be detected in a sample from the subject (e.g., detecting a BRAF gene having a mutation corresponding to a V600E mutation in BRaf protein and/or detecting a BRaf protein having a V600E mutation). In some embodiments, the subject was determined (e.g., prior to administration of a compound provided herein) to have a cancer having a BRaf V600E mutation In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant CRC (e.g., BRaf V600E mutant CRC), and the additional therapy or therapeutic agent is a BRaf inhibitor (e.g., avutometinib (R05126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and an anti-EGFR antibody or anti-EGFR antibody-drug conjugate (e.g., amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, TAS-0313, or biosimilars thereof). In some embodiments, the cancer is a BRaf mutant CRC (e.g., BRaf V600E mutant CRC), and the additional therapy or therapeutic agent is a BRaf inhibitor (e.g., avutometinib (R05126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and cetuximab (e.g., ERBITUX® (cetuximab), or a biosimilar thereof (e.g., CMAB-009, CPGJ-602, or KL-140)).

In some embodiments, the cancer is a BRaf mutant CRC (e.g., BRaf V600E mutant CRC), and the additional therapy or therapeutic agent is a BRaf inhibitor (e.g., avutometinib (R05126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436), encorafenib (e.g., BRAFTOVI™ LGX818), or vemurafenib (e.g., ZELBORAF®, R05185426) and cetuximab (e.g., ERBITUX® (cetuximab), or a biosimilar thereof (e.g., CMAB-009, CPGJ-602, or KL-140)).

In some embodiments, the cancer is a BRaf mutant CRC (e.g., BRaf V600E mutant CRC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, R05185426) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436) and panitumumab (e.g., VECTIBIX® (panitumumab), or a biosimilar thereof).

In some embodiments, the cancer is a BRaf mutant NSCLC (e.g., BRaf V600E mutant NSCLC), and the additional therapy or therapeutic agent is a BRaf inhibitor (e.g., avutometinib (R05126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, R05185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant NSCLC (e.g., BRaf V600E mutant NSCLC), and the additional therapy or therapeutic agent is vemurafenib (e.g., ZELBORAF®, R05185426), and cobimetinib (e.g., cobimetinib fumarate). In some embodiments, the cancer is a BRaf mutant NSCLC (e.g., BRaf V600E mutant NSCLC), and the additional therapy or therapeutic agent is encorafenib (e.g., BRAFTOVI™, LGX818) and binimetinib.

In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, R05185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant melanoma (e.g., BRaf V600E mutant melanoma), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF@, R05185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant melanoma (e.g., BRaf V600E mutant melanoma), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant melanoma (e.g., BRaf V600E mutant melanoma), and the additional therapy or therapeutic agent is vemurafenib (e.g., ZELBORAF®, RO5185426), and cobimetinib (e.g., cobimetinib fumarate). In some embodiments, the cancer is a BRaf mutant melanoma (e.g., BRaf V600E mutant melanoma), and the additional therapy or therapeutic agent is encorafenib (e.g., BRAFTOVI™ LGX818) and binimetinib. In some such embodiments, the subject has previously been treated with an immunotherapy.

In some embodiments, the CDK2 inhibitor is ebvaciclib, fadraciclib, milciclib, pacritinib (e.g., pacritinib citrate), roniciclib, roscovitine, BLU-222, NUV-422, PF-07104091, TQB-3616, or a combination thereof.

In some embodiments, the CDK4/6 inhibitor is abemaciclib, birociclib, dalpiciclib, lerociclib, milciclib, palbociclib, ribociclib (e.g., ribociclib succinate), riviciclib, roniciclib, trilaciclib (e.g., trilaciclib dihydrochloride), FCN-437, TQB-3616, or a combination thereof.

In some embodiments, the CDK7 inhibitor is milciclib, roscovitine, samuraciclib, or a combination thereof.

In some embodiments, the CDK9 inhibitor is fadraciclib, riviciclib, roniciclib, roscovitine, zotiraciclib, AZD-4573, KB-0742, or a combination thereof.

In some embodiments, the EGFR inhibitor is abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, an anti-EGFR antibody or anti-EGFR antibody-drug conjugate, or a combination thereof.

In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, TAS-0313, biosimilars thereof, or a combination thereof. In some embodiments, the anti-EGFR antibody or anti-EGFR antibody-drug conjugates is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI—B-001, biosimilars thereof, or a combination thereof.

In some embodiments, an EGFR mutation (e.g., an EGFR exon 19 deletion or an EGFR L858R mutation (with or without an EGFR T790M mutation)) can be detected in a sample from the subject (e.g., detecting an EGFR gene having a mutation (e.g., a mutation corresponding to an EGFR exon 19 deletion or an EGFR L858R mutation (with or without an EGFR T790M mutation) in an EGFR protein) and/or detecting an EGFR protein having a mutation (e.g., an EGFR exon 19 deletion or an EGFR L858R mutation (with or without an EGFR T790M mutation))). In some embodiments, the subject was determined (e.g., prior to administration of a compound provided herein) to have a cancer having an EGFR mutation (e.g., an EGFR exon 19 deletion or an EGFR L858R mutation (with or without an EGFR T790M mutation)).

In some embodiments, the cancer is an EGFR mutant NSCLC (e.g., EGFR exon 19 deletion NSCLC or EGFR L858R (with or without T790M) mutant NCLC), and the additional therapy or therapeutic agent is an EGFR inhibitor (e.g., abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, or an anti-EGFR antibody or anti-EGFR antibody-drug conjugate). In some embodiments, the cancer is an EGFR mutant NSCLC (e.g., EGFR exon 19 deletion NSCLC or EGFR L858R (with or without T790M) mutant NCLC), and the additional therapy or therapeutic agent is osimertinib (e.g., osimertinib mesylate).

In some embodiments, the ERK inhibitor is rineterkib, temuterkib, ulixertinib, ASN-0007, ASTX-029, ATG-017, BPI-27336, HH-2710, JSI-1187, MK-8353, or a combination thereof.

In some embodiments, the EZH2 inhibitor is lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, XNW-5004, or a combination thereof. In some embodiments, the EZH2 inhibitor also inhibits EZH1 (also referred to as an EZH1/2 inhibitor).

In some embodiments, the cancer is peripheral T cell lymphoma, and the additional therapy or therapeutic agent is an EZH2 inhibitor (e.g., lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, or XNW-5004).

In some embodiments, the FGFR1 inhibitor is danusertib, dovitinib, erdafitinib, futibatinib, infigratinib (e.g., infigratinib phosphate), lenvatinib (e.g., lenvatinib mesylate), lucitanib, nintedanib (e.g., nintedanib esylate), pemigatinib, surufatinib, tasurgratinib, tinengotinib, zoligratinib, FH-2001, HMPL-453, LY-2874455, or a combination thereof.

In some embodiments, the FGFR2 inhibitor is erdafitinib, futibatinib, infigratinib (e.g., infigratinib phosphate), lucitanib, pemigatinib, tasurgratinib, zoligratinib, bemarituzumab (or biosimilars thereof), FH-2001, HMPL-453, LY-2874455, or a combination thereof.

In some embodiments, the FGFR3 inhibitor is dovitinib, erdafitinib, futibatinib, infigratinib (e.g., infigratinib phosphate), lucitanib, masitinib, nintedanib, pemigatinib, tasurgratinib, zoligratinib, vofatamab (or biosimilars thereof), EXEL-0999, FH-2001, HMPL-453, LY-2874455, or a combination thereof.

In some embodiments, the FGFR4 inhibitor is axitinib, erdafitinib, futibatinib, infigratinib (e.g., infigratinib phosphate), irpagratinib, nintedanib, pemigatinib, FH-2001, H3B-6527, LY-2874455, or a combination thereof.

In some embodiments, the HER2 inhibitor is afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, an anti-HER2 antibody or anti-HER2 antibody-drug conjugate, or a combination thereof.

In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, biosimilars thereof, or a combination thereof. In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, TAA-013, SHR-A1811, BAT-8001, MDX-210, Alpha-Her2-pAF1-AS-269, MRG-002, DF-1001, AC-101, MM-111, biosimilars thereof, or a combination thereof.

In some embodiments, HER2+ status can be detected in a sample from the subject (e.g., via immunohistochemistry (IHC) and/or fluorescent in situ hybridization (FISH)). In some embodiments, the subject was determined (e.g., prior to administration of a compound provided herein) to have a cancer with HER2+ status.

In some embodiments, HER2 low status can be detected in a sample from the subject (e.g., via IHC and/or FISH). In some embodiments, the subject was determined (e.g., prior to administration of a compound provided herein) to have a cancer with HER2 low status.

In some embodiments, HER2− status can be detected in a sample from the subject (e.g., via IHC and/or FISH). In some embodiments, the subject was determined (e.g., prior to administration of a compound provided herein) to have a cancer with HER2− status.

In some embodiments, ER expression status can be detected in a sample from the subject (e.g., via IHC and/or FISH). In some embodiments, the subject was determined (e.g., prior to administration of a compound provided herein) to have a cancer with ER expression.

In some embodiments, the subject was determined (e.g., prior to administration of a compound provided herein) to have a cancer without ER expression.

In some embodiments, PR expression status can be detected in a sample from the subject (e.g., via IHC, and/or FISH). In some embodiments, the subject was determined (e.g., prior to administration of a compound provided herein) to have a cancer with PR expression.

In some embodiments, the subject was determined (e.g., prior to administration of a compound provided herein) to have a cancer without PR expression.

In some embodiments, the cancer is a HER2+ breast cancer (e.g., HER2+ breast cancer with ER expression, HER2+ breast cancer without ER expression), and the additional therapy is a HER2 inhibitor (e.g., afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, or an anti-HER2 antibody or anti-HER2 antibody-drug conjugate). In some embodiments, the cancer is a HER2+ breast cancer (e.g., HER2+ breast cancer with ER expression, HER2+ breast cancer without ER expression), and the additional therapy is tucatinib. In some embodiments, the cancer is a HER2+ breast cancer (e.g., HER2+ breast cancer with ER expression, HER2+ breast cancer without ER expression), and the additional therapy is an anti-HER2 antibody or anti-HER2 antibody-drug conjugate (e.g., anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, or biosimilars thereof). In some embodiments, the cancer is a HER2+ breast cancer (e.g., HER2+ breast cancer with ER expression, HER2+ breast cancer without ER expression), and the additional therapy is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)). In some embodiments, the cancer is a HER2+ breast cancer (e.g., HER2+ breast cancer with ER expression, HER2+ breast cancer without ER expression), and the additional therapy is trastuzumab deruxtecan (e.g., fam-trastuzumab deruxtecan-nxki, or a biosimilar thereof). In some embodiments, the cancer is a HER2+ breast cancer (e.g., HER2+ breast cancer with ER expression, HER2+ breast cancer without ER expression), and the additional therapy is trastuzumab emtansine, or a biosimilar thereof. In some embodiments, the cancer is a HER2 low breast cancer, and the additional therapy is an anti-HER2 antibody or anti-HER2 antibody-drug conjugate. In some embodiments, the cancer is a HER2 low breast cancer, and the additional therapy is trastuzumab deruxtecan (e.g., fam-trastuzumab deruxtecan-nxki, or a biosimilar thereof).

In some embodiments, the JAK inhibitor is adelatinib, baricitinib, brepocitinib, deuruxolitinib, fedratinib (e.g., fedratinib dihydrochloride monohydrate), filgotinib (e.g., filgotinib maleate), gandotinib, gusacitinib, ilginatinib, izencitinib, jaktinib, momelotinib (e.g., momelotinib dihydrochloride), nezulcitinib, pacritinib (e.g., pacritinib citrate), peficitinib (e.g., peficitinib hydrobromide), ropsacitinib, ruxolitinib (e.g., ruxolitinib phosphate), tasocitinib (e.g., tofacitinib citrate), AT-9283, TQ-05105, or a combination thereof. In some embodiments, the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof. In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate).

In some embodiments, a JAK V617F mutation can be detected in a sample from the subject (e.g., detecting a JAK2 gene having a mutation corresponding to a V617F mutation in JAK2 protein and/or detecting a JAK2 protein having a V617F mutation). In some embodiments, the subject was determined (e.g., prior to administration of a compound provided herein) to have a cancer having a JAK2 V617F mutation. In some embodiments, the subject was determined (e.g., prior to administration of a compound provided herein) to have a cancer lacking a JAK2 V617F mutation.

In some embodiments, the cancer is post-MPN AML, M6-AML, or M7-AML, and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the patient has received a previous line of therapy including a JAK inhibitor. In some embodiments, the patient has not received a previous line of therapy including a JAK inhibitor.

In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate). In some embodiments, the patient has received a previous line of therapy including a JAK inhibitor. In some embodiments, the patient has not received a previous line of therapy including a JAK inhibitor. In some embodiments, treatment effect can be measured by Spleen Volume Reduction (e.g., Spleen Volume Reduction of greater than or equal to 35% (SVR₃₅), for example, measured by MRI or CT), Total Symptom Score (e.g., Total Symptom Score reduction of greater than or equal to 50% (TSS₅₀), for example, measured by the Myelofibrosis Symptom Assessment Form (MFSAF) version 4.0), or both, such as at 24 weeks after beginning of treatment; see, e.g., Harrison, Claire, et al., New England Journal of Medicine 366.9 (2012): 787-798, doi: 10.1056/NEJMoa1110556; and Verstovsek, Srdan, et al. New England Journal of Medicine 366.9 (2012): 799-807, doi: 10.1056/NEJMoa1110557.

In some embodiments, treatment effect can be measured (e.g., in addition to or instead of SVR₃₅ and/or TSS₅₀) by anemia response (e.g., measured by current International Working Group-Myeloproliferative Neoplasms Research and European LeukemiaNet (IWG-MRT/ELN) criteria), bone marrow fibrosis (e.g., according to the European Consensus Grading System through bone marrow biopsy, such as at 24 or 96 weeks after beginning of treatment), variant allele fraction (e.g., JAK2 V617F variant allele fraction), transfusion independence, overall survival, leukemia-free survival, change in physical functioning (e.g., measured by the physical functioning domain of the European Organization for Research and Treatment of Cancer (EORTC) Quality of Life Questionnaire (QLQ)-C30 or death), change in fatigue (e.g., assessed using the Patient-Reported Outcomes Measurement Information System (PROMIS) Fatigue SF 7a), or a combination thereof.

In some embodiments, the KRas inhibitor is adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, RSC-1255, or a combination thereof. Additional KRas inhibitors are disclosed in, for example, International Publication Nos. WO 2024/040131 and WO 2024/112654.

In some embodiments, a KRas mutation (e.g., a KRas G12C mutation or a KRas G12D mutation) can be detected in a sample from the subject (e.g., detecting a KRAS gene having a mutation corresponding to a G12C mutation or a G12D mutation in KRas protein and/or detecting a KRas protein having a G12C mutation or a G12D mutation). In some embodiments, the subject was determined (e.g., prior to administration of a compound provided herein) to have a cancer having a KRas G12C mutation. In some embodiments, the subject was determined (e.g., prior to administration of a compound provided herein) to have a cancer having a KRas G12D mutation.

In some embodiments, the cancer is a KRas mutant lung cancer (e.g., a KRas mutant NSCLC), a KRas mutant CRC, or a KRas mutant pancreatic cancer, and the additional therapy or therapeutic agent is a KRas inhibitor (e.g., adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, or RSC-1255). In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is adagrasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is divarasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is sotorasib. In some embodiments, the cancer is a KRas mutant lung cancer (e.g., a KRas mutant NSCLC (e.g., a KRas G12D mutant NSCLC)), a KRas mutant CRC (e.g., a KRas G12D mutant CRC), or a KRas mutant pancreatic cancer (e.g., a KRas G12D mutant pancreatic cancer), and the additional therapy or therapeutic agent is MRTX1133.

In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, TAK-733, or a combination thereof.

In some embodiments, a BRCA1 mutation can be detected in a sample from the subject (e.g., detecting a BRCAI gene having a mutation and/or detecting a BRCA1 protein having a mutation). In some embodiments, the subject was determined (e.g., prior to administration of a compound provided herein) to have a cancer having a BRCA1 mutation.

In some embodiments, a BRCA2 mutation can be detected in a sample from the subject (e.g., detecting a BRCA2 gene having a mutation and/or detecting a BRCA2 protein having a mutation). In some embodiments, the subject was determined (e.g., prior to administration of a compound provided herein) to have a cancer having a BRCA2 mutation.

In some embodiments, the cancer is ovarian cancer (e.g., BRCA1 mutant ovarian cancer or BRCA2 mutant ovarian cancer, HGSOC (e.g., BRCA1 mutant HGSOC or BRCA2 mutant HGSOC), or LGSOC), and the additional therapy or therapeutic agent is a MEK inhibitor (e.g., binimetinib, cobimetinib, selumetinib, or trametinib). In some embodiments, the cancer is BRCA1 mutant ovarian cancer, and the additional therapy or therapeutic agent is a MEK inhibitor (e.g., binimetinib, cobimetinib, selumetinib, or trametinib). In some embodiments, the cancer is BRCA2 mutant ovarian cancer, and the additional therapy or therapeutic agent is a MEK inhibitor (e.g., binimetinib, cobimetinib, selumetinib, or trametinib). In some embodiments, the cancer is HGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib). In some embodiments, the cancer is LGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib).

In some embodiments, the cancer is a KRas mutant CRC (e.g., a KRas G12C mutant NSCLC), and the additional therapy or therapeutic agent is a MEK inhibitor (e.g., binimetinib, cobimetinib, selumetinib, or trametinib).

In some embodiments, the MET inhibitor is cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, TQ-B-3139, or a combination thereof.

In some embodiments, a MET alteration can be detected in a sample from the subject (e.g., detecting aMET gene having an alteration (e.g., gene amplification or exon14 skipping) and/or detecting a MET protein having a mutation (e.g., exon14 skipping)). In some embodiments, the subject was determined (e.g., prior to administration of a compound provided herein) to have a cancer having a MET alteration.

In some embodiments, the cancer is a MET-altered NCLC (e.g., MET amplified NSCLC or MET exon14 skipping NSCLC), and the additional therapy or therapeutic agent is a MET inhibitor (e.g., cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, or TQ-B-3139). In some embodiments, the cancer is a MET-altered NCLC (e.g., MET amplified NSCLC or MET exon14 skipping NSCLC), and the additional therapy or therapeutic agent is capmatinib (e.g., capmatinib hydrochloride) or tepotinib (e.g., tepotinib hydrochloride hydrate). In some embodiments, the cancer is a MET-altered NCLC (e.g., MET amplified NSCLC or MET exon14 skipping NSCLC), and the additional therapy or therapeutic agent is telisotuzumab vedotin (or biosimilars thereof).

In some embodiments, the Hif2a inhibitor is belzutifan, AB-521, DFF-332, NKT-2152, PT-2399, or a combination thereof.

In some embodiments, the PARP inhibitor is fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, NMS-03305293, or a combination thereof. In some embodiments, the PARP inhibitor is a PARP1 inhibitor. In some embodiments, the PARP1 inhibitor is saruparib (AZD5305), NMS-03305293, or a combination thereof.

In some embodiments, the cancer is BRCA1 mutant breast cancer or BRCA2 mutant breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is BRCA1 mutant breast cancer or BRCA2 mutant breast cancer, and the additional therapy or therapeutic agent is saruparib.

In some embodiments, the cancer is triple negative breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is triple negative breast cancer, and the additional therapy or therapeutic agent is saruparib.

In some embodiments, the cancer is HGSOC (e.g., BRCA1 mutant HGSOC or BRCA2 mutant HGSOC), and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is HGSOC (e.g., BRCA1 mutant HGSOC or BRCA2 mutant HGSOC), and the additional therapy or therapeutic agent is saruparib.

In some embodiments, the LSD1 inhibitor is bomedemstat, iadademstat, pulrodemstat, seclidemstat (HCI-2577), vafidemstat, GSK-2879552, INCB-059872, JBI-802, or a combination thereof.

In some embodiments, the BET inhibitor is alobresib, apabetalone, mivebresib, pelabresib, trotabresib, ABBV-744, BI-2536, BMS-986158, INCB-057643, JAB-8263, ODM-207, PLX-2853, ZEN-003694, or a combination thereof.

In some embodiments, the cancer is myelofibrosis (e.g., primary myelofibrosis, post-essential thrombocythemia myelofibrosis, or post-polycythemia vera myelofibrosis), and the additional therapy or therapeutic agent is a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694).

In some embodiments, the STING agonist is ulevostinag, ADU-S100, or a combination thereof.

In some embodiments, the telomerase inhibitor is imetelstat (e.g., imetelstat sodium).

In some embodiments, the TORC1/2 inhibitor is apitolisib, bimiralisib, dactolisib, deforolimus, everolimus, fosciclopirox (e.g., fosciclopirox sodium), gedatolisib, onatasertib, paxalisib, sapanisertib, sirolimus, sodium 2-hydroxylinoleate, temsirolimus, umirolimus, vistusertib, zandelisib, zotarolimus, BI-860585, CC-115, CLL-442, PF-04691502, or a combination thereof.

In some embodiments, the VEGFR inhibitor is apatinib, axitinib, cabozantinib (e.g., cabozantinib S-malate), catequentinib (alontinib), cediranib, dovitinib, famitinib, fruquintinib, glesatinib, ibcasertib, ilorasertib, lenvatinib (e.g., lenvatinib mesylate), lucitanib, nintedanib (e.g., nintedanib esylate), pamufetinib, pazopanib (e.g., pazopanib hydrochloride), regorafenib (e.g., regorafenib monohydrate), sitravatinib, sorafenib (e.g., sorafenib tosylate), sunitinib (e.g., sunitinib malate), surufatinib (sulfatinib), telatinib, tinengotinib, tivozanib (e.g., tivozanib hydrochloride monohydrate), vandetanib, vorolanib, zanzalintinib, olinvacimab (or biosimilars thereof), ramucirumab (or biosimilars thereof), CEP-11981, ENMD-2076, ODM-203, or a combination thereof.

In some embodiments, the chemotherapy is a platinum complex, a microtubule inhibitor (e.g., a microtubule destabilizer or a microtubule stabilizer), a topoisomerase inhibitor, a hypomethylating agent, or an antibody-drug conjugate including any thereof. In some embodiments, the platinum complex is carboplatin, cisplatin, lobaplatin, miriplatin, oxaliplatin, or a combination thereof. In some embodiments, the microtubule inhibitor is cabazitaxel, colchicine, desoxyepothilone B, docetaxel, eribulin, ixabepilone, nab-paclitaxel, paclitaxel, plinabulin, sabizabulin, tirbanibulin, vinblastine, vinflunine, vinorelbine, or a combination thereof. In some embodiments, the microtubule inhibitor is cabazitaxel, docetaxel, nab-paclitaxel, paclitaxel, or a combination thereof. In some embodiments, the topoisomerase inhibitor is aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, SN-38, or a combination thereof. In some embodiments, the hypomethylating agent is azacitidine, decitabine, or a combination thereof. In some embodiments, the chemotherapy is a platinum complex and a topoisomerase inhibitor (e.g., cisplatin and etoposide). In some embodiments, the antibody-drug conjugate including the microtubule inhibitor is belantamab mafodotin, brentuximab vedotin, cofetuzumab pelidotin, disitamab vedotin, enfortumab vedotin (e.g., enfortumab vedotin-ejfv, or a biosimilar thereof), mirvetuximab soravtansine (e.g., mirvetuximab soravtansine-gynx, or a biosimilar thereof), polatuzumab vedotin, telisotuzumab vedotin, tisotumab vedotin, trastuzumab emtansine (e.g., ado-trastuzumab emtansine, or a biosimilar thereof), tusamitamab ravtansine, upifitamab rilsodotin, zilovertamab vedotin, Alpha-Her2-pAF1-AS-269, BAT-8001, TAA-013, biosimilars thereof, or a combination thereof. In some embodiments, the antibody-drug conjugate including the microtubule inhibitor is enfortumab vedotin (e.g., enfortumab vedotin-ejfv, or a biosimilar thereof). In some embodiments, the antibody-drug conjugate including the microtubule inhibitor is mirvetuximab soravtansine (e.g., mirvetuximab soravtansine-gynx, or a biosimilar thereof). In some embodiments, the antibody-drug conjugate including the microtubule inhibitor is trastuzumab emtansine (e.g., ado-trastuzumab emtansine, or a biosimilar thereof).

In some embodiments, the antibody-drug conjugate including the topoisomerase inhibitor is datopotamab deruxtecan, patritumab deruxtecan, sacituzumab govitecan (e.g., sacituzumab govitecan-hziy, or a biosimilar thereof), trastuzumab deruxtecan (fam-trastuzumab deruxtecan-nxki, or a biosimilar thereof), or a combination thereof. In some embodiments, the antibody-drug conjugate including the topoisomerase inhibitor is sacituzumab govitecan (e.g., sacituzumab govitecan-hziy, or a biosimilar thereof). In some embodiments, the antibody-drug conjugate including the topoisomerase inhibitor is trastuzumab deruxtecan (e.g., fam-trastuzumab deruxtecan-nxki, or a biosimilar thereof).

In some embodiments, the cancer is a lung cancer (e.g., NSCLC (e.g., squamous cell carcinoma)), and the additional therapy or therapeutic agent is a microtubule inhibitor (e.g., cabazitaxel, docetaxel, nab-paclitaxel, or paclitaxel). In some embodiments, the cancer is a lung cancer (e.g., NSCLC (e.g., squamous cell carcinoma)), and the additional therapy or therapeutic agent is docetaxel.

In some embodiments, the cancer is NSCLC (e.g., NSCLC with a MET amplification), and the additional therapy or therapeutic agent is telisotuzumab vedotin.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD1 therapy.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD-L1 therapy.

In some embodiments, the cancer is a lung cancer (e.g., NSCLC), and the additional therapy or therapeutic agent is a pemetrexed and anti-PD1 therapy.

In some embodiments, the cancer is a lung cancer (e.g., NSCLC), and the additional therapy or therapeutic agent is a pemetrexed and anti-PD-L1 therapy.

In some embodiments, the cancer is a lung cancer (e.g., SCLC), and the additional therapy or therapeutic agent is a platinum complex (e.g., carboplatin, cisplatin, lobaplatin, miriplatin, or oxaliplatin) and a topoisomerase inhibitor (e.g., aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, or SN-38). In some embodiments, the cancer is a lung cancer (e.g., SCLC), and the additional therapy or therapeutic agent is carboplatin and etoposide. In some embodiments, the cancer is a lung cancer (e.g., SCLC), and the additional therapy or therapeutic agent is cisplatin and etoposide.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is nab-paclitaxel or paclitaxel.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD-L1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is capecitabine.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is sacituzumab govitecan (e.g., sacituzumab govitecan-hziy, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab deruxtecan (e.g., fam-trastuzumab deruxtecan-nxki, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)).

In some embodiments, the cancer is hormone receptor positive breast cancer, and the additional therapy or therapeutic agent is hormone therapy (e.g., tamoxifen, toremifene, or a combination thereof).

In some embodiments, the cancer is hormone receptor positive breast cancer, and the additional therapy or therapeutic agent is a selective estrogen receptor degrader (SERD) (e.g., fulvestrant, elacestrant, or a combination thereof).

In some embodiments, the cancer is a HER2+ breast cancer (e.g., HER2+ breast cancer with ER expression, HER2+ breast cancer without ER expression), and the additional therapy or therapeutic agent is trastuzumab deruxtecan (e.g., fam-trastuzumab deruxtecan-nxki, or a biosimilar thereof) or trastuzumab emtansine (e.g., KADCYLA® (trastuzumab emtansine), or a biosimilar thereof (e.g., UJVIRA® (trastuzumab emtansine))).

In some embodiments, the cancer is a HER2 low breast cancer, and the additional therapy or therapeutic agent is trastuzumab deruxtecan (e.g., fam-trastuzumab deruxtecan-nxki, or a biosimilar thereof).

In some embodiments, the cancer is a head and neck cancer, and the additional therapy or therapeutic agent is a microtubule inhibitor (e.g., cabazitaxel, docetaxel, nab-paclitaxel, or paclitaxel). In some embodiments, the cancer is a head and neck cancer, and the additional therapy or therapeutic agent is docetaxel.

In some embodiments, the cancer is a cervical cancer, and the additional therapy or therapeutic agent is a microtubule inhibitor (e.g., cabazitaxel, docetaxel, nab-paclitaxel, or paclitaxel).

In some embodiments, the cancer is an endometrial cancer, and the additional therapy or therapeutic agent is a microtubule inhibitor (e.g., cabazitaxel, docetaxel, nab-paclitaxel, or paclitaxel).

In some embodiments, the cancer is a prostate cancer, and the additional therapy or therapeutic agent is a microtubule inhibitor (e.g., cabazitaxel, docetaxel, nab-paclitaxel, or paclitaxel).

In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel). In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel) and a platinum complex (e.g., carboplatin, cisplatin, lobaplatin, miriplatin, or oxaliplatin). In some embodiments, the cancer is an ovarian cancer (e.g., HGSOC) and the additional therapy or therapeutic agent is nab-paclitaxel or paclitaxel and carboplatin.

In some embodiments, the cancer is a pancreatic cancer, and the additional therapy or therapeutic agent is a microtubule inhibitor (e.g., cabazitaxel, docetaxel, nab-paclitaxel, or paclitaxel). In some embodiments, the cancer is a pancreatic cancer, and the additional therapy or therapeutic agent is a microtubule inhibitor (e.g., cabazitaxel, docetaxel, nab-paclitaxel, or paclitaxel) and gemcitabine.

In some embodiments, the cancer is a bladder cancer (e.g., bladder urothelial carcinoma), and the additional therapy or therapeutic agent is enfortumab vedotin (e.g., enfortumab vedotin-ejfv, or a biosimilar thereof).

In some embodiments, the cancer is a bladder cancer (e.g., bladder urothelial carcinoma), and the additional therapy or therapeutic agent is sacituzumab govitecan (e.g., sacituzumab govitecan-hziy, or a biosimilar thereof).

In some embodiments, the cancer is triple negative breast cancer, and the additional therapy or therapeutic agent is sacituzumab govitecan (e.g., sacituzumab govitecan-hziy, or a biosimilar thereof).

In some embodiments, folate receptor positivity status can be detected in a sample from the subject (e.g., via immunohistochemistry (IHC) and/or fluorescent in situ hybridization (FISH)). In some embodiments, the subject was determined (e.g., prior to administration of a compound provided herein) to have a cancer that is folate receptor positive.

In some embodiments, the cancer is folate receptor positive ovarian cancer (e.g., folate receptor positive HGSOC), and the additional therapy or therapeutic agent is mirvetuximab soravtansine (e.g., mirvetuximab soravtansine-gynx, or a biosimilar thereof).

In some embodiments the immunomodulatory imide drug is thalidomide, lenalidomide, pomalidomide, iberdomide, avadomide, CC-99282, or a combination thereof.

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, biosimilars thereof, or a combination thereof.

In some embodiments, the PD-L1 inhibitor is INCB-086550.

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., IMFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof.

In some embodiments, the additional therapy is radiotherapy.

In some embodiments, the cancer is head and neck cancer (e.g., head and neck squamous cell carcinoma), and the additional therapy is radiotherapy.

In some embodiments, the additional therapy includes a BRaf inhibitor and a MEK inhibitor. For example, the additional therapy can include dabrafenib and trametinib, vemurafenib and cobimetinib, or encorafenib and binimetinib.

Exemplary descriptions of agents in combination with BCL-2 family inhibitors can be found in: Hikita, Hayato, et al. Hepatology 52.4 (2010): 1310-1321, doi: 10.1002/hep.23836; Chen, Jun, et al. Molecular Cancer Therapeutics 10.12 (2011): 2340-2349, doi: 10.1158/1535-7163.MCT-11-0415; Inuzuka, Hiroyuki, et al. Nature 471.7336 (2011): 104-109, doi: 10.1038/nature09732; Wertz, Ingrid E., et al. Nature 471.7336 (2011): 110-114, doi: 10.1038/nature09779; Tan, Nguyen, et al. Clinical Cancer Research 17.6 (2011): 1394-1404, doi: 10.1158/1078-0432.CCR-10-2353; Wong, Maureen, et al. Molecular Cancer Therapeutics 11.4 (2012): 1026-1035, doi: 10.1158/1535-7163.MCT-11-0693; Corcoran, R^{Ya} n B., et al. Cancer Cell 23.1 (2013): 121-128, doi: 10.1016/j.ccr.2012.11.007; Waibel, Michaela, et al. Cell Reports 5.4 (2013): 1047-1059, doi: 10.1016/j.celrep.2013.10.038; Frederick, Dennie T., et al. PloS One 9.7 (2014): e101286, doi: 10.1371/journal.pone.0101286; Vlahovic, Gordana, et al. Investigational New Drugs 32.5 (2014): 976-984, doi: 10.1007/s10637-014-0116-3; Faber, Anthony C., et al. Cancer Discovery 4.1 (2014): 42-52, doi: 10.1158/2159-8290.CD-13-0315; Leverson, Joel D., et al. Science Translational Medicine 7.279 (2015): 279ra40-279ra40, doi: 10.1126/scitranslmed.aaa4642; Guo, Jun, et al. PLoS One 10.3 (2015): e0114363, doi: 10.1371/journal.pone.0114363; Lheureux, Stephanie, et al. International Journal of Cancer 136.5 (2015): E340-E350, doi: 10.1002/ijc.29104; Zoeller, Jason J., et al. Cancer Research 76.14_Supplement (2016): 4358-4358, doi: 10.1158/1538-7445.AM2016-4358; Weeden, Clare E., et al. Oncogene 37.32 (2018): 4475-4488, doi: 10.1038/s41388-018-0268-2; Lucantoni, Federico, et al. Cell Death & Disease 9.2 (2018): 1-13, doi: 10.1038/s41419-017-0039-y; Iavarone, Claudia, et al. Molecular Cancer Therapeutics 18.3 (2019): 642-655, doi: 10.1158/1535-7163.MCT-18-0413; Fleury, Hubert, et al. Nature Communications 10.1 (2019): 2556, doi: 10.1038/s41467-019-10460-1; Lohard, Steven, et al. Nature Communications 11.1 (2020): 259, doi: 10.1038/s41467-019-13689-y; Bertino, Erin M., et al. Clinical Cancer Research 27.6 (2021): 1604-1611, doi: 10.1158/1078-0432.CCR-20-4084; Guo, Ting, et al. Aging 13.15 (2021): 19750, doi: 10.18632/aging.203386; Puglisi, Martina, et al. Future Oncology 17.21 (2021): 2747-2758, doi: 10.2217/fon-2021-0140; Harrison, Claire N., et al. Journal of Clinical Oncology 40.15 (2022): 1671, doi: 10.1200/JCO.21.02188; Kohler, Jens, et al. Molecular Cancer Therapeutics 20.4 (2021): 641-654, doi: 10.1158/1535-7163.MCT-20-0531; Jaaks, Patricia, et al. Nature 603.7899 (2022): 166-173, doi: 10.1038/s41586-022-04437-2; Sobol, Benjamin, et al. International Journal of Molecular Sciences 23.14 (2022): 7850, doi: 10.3390/ijms23147850; Passamonti, J Clin Oncol 40, (2022) (suppl 16; abstr 7015), doi: 10.1200/JCO.2022.40.16_suppl.7015; Qin, JCiin Oncol 40, (2022) (suppl 16; abstr e20612), doi: 10.1200/JCO.2022.40.16_suppl.e20612; Potter, Danielle S., et al. Cancer Research 82.12_Supplement (2022): 3691-3691, doi: 10.1158/1538-7445.AM2022-3691; and Shebl, Bassem, et al. Cancer Research 82.12_Supplement (2022): 3888-3888, doi: 10.1158/1538-7445.AM2022-3888.

Also provided herein is a method of treating cancer, comprising administering to a subject in need thereof (α) a compound provided herein, or a pharmaceutically acceptable salt thereof, and (b) an additional therapeutic agent, for simultaneous, separate or sequential use for the treatment of cancer, wherein the amounts of the compound provided herein, or a pharmaceutically acceptable salt thereof, and the additional therapeutic agent are together effective in treating the cancer. In some embodiments, the method comprises administering (c) at least one pharmaceutically acceptable carrier.

These additional therapeutic agents may be administered with one or more doses of the compound provided herein, or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art. In some embodiments, the compound provided herein, or a pharmaceutically acceptable salt thereof, and the additional therapeutic agent are administered simultaneously as separate dosages. In some embodiments, a compound provided herein, or a pharmaceutically acceptable salt thereof, and the additional therapeutic agent are administered as separate dosages sequentially in any order, in jointly therapeutically effective amounts, e.g., in daily or intermittently dosages. In some embodiments, the compound provided herein, or a pharmaceutically acceptable salt thereof, and the additional therapeutic agent are administered simultaneously as a combined dosage.

Also provided herein is (i) a pharmaceutical combination for treating a cancer in a subject in need thereof, which comprises (α) a compound provided herein, or a pharmaceutically acceptable salt thereof, and (b) at least one additional therapeutic agent (e.g., any of the exemplary additional therapeutic agents described herein or known in the art), for simultaneous, separate or sequential use for the treatment of cancer, wherein the amounts of the compound provided herein, or a pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the pharmaceutical combination comprises (c) at least one pharmaceutically acceptable carrier.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound provided herein, or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

The phrase “effective amount” of a compound provided herein, as used herein, means an amount of the compound that, when administered to a cell, in vitro or in vivo, is sufficient to reduce proliferation of the cell or to kill the cell. The amount of a compound provided herein, or a pharmaceutically acceptable salt thereof, that will correspond to such an amount will vary depending upon factors such as the particular compound and genetics of the cell to be treated, but can nevertheless be routinely determined by one skilled in the art.

### Pharmaceutical Compositions and Administration

### General

In some embodiments, a compound provided herein, or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 1%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound provided herein, or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, intravitreal, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound provided herein, or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795, doi: 10.1593/neo.06436.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, doi: 10.2174/1568026611313070002.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a compound provided herein is administered to an individual for a period of time followed by a separate period of time (e.g., a dosing holiday). In another embodiment, a compound provided herein is administered for a first period and a second period following the first period, with administration stopped during the second period (e.g., a dosing holiday), followed by a third period where administration of the compound provided herein is started and then a fourth period following the third period where administration is stopped (e.g., a dosing holiday). In an aspect of this embodiment, the period of administration of a compound provided herein followed by a period where administration is stopped (e.g., a dosing holiday) is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped (e.g., a dosing holiday) is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts can be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

In some embodiments, a pharmaceutically acceptable salt is an anion salt selected from the group consisting of: a mesylate salt, a besylate salt, an acetate salt, a benzenesulfonate salt, a benzoate salt, a bicarbonate salt, a bitartrate salt, a bromide salt, a calcium edetate salt, a camsylate salt, a carbonate salt, a chloride salt, a citrate salt, a dihydrochloride salt, an edetate salt, an edisylate salt, an estolate salt, an esylate salt, a fumarate salt, a gluceptate salt, a gluconate salt, a glucuronate salt, a glutamate salt, a glycollylarsanilate salt, a hexylresorcinol salt, a hydramine salt, a hydrobromide salt, a hydrochloride salt, a hydroxynaphthoate salt, an iodide salt, an isethionate salt, a lactate salt, a lactobionate salt, a malate salt, a maleate salt, a mandelate salt, a mesylate salt, a methylbromide salt, a methylnitrate salt, a methylsulfate salt, a mucate salt, a napsylate salt, a nitrate salt, a pamoate (embonate) salt, a pantothenate salt, a phosphate/diphosphate salt, a polygalacturonate salt, a salicylate salt, a stearate salt, a subacetate salt, a succinate salt, a sulfate salt, an oleate salt, a tannate salt, a tartrate salt, a teoclate salt, and a triethiodide salt.

In some embodiments, a pharmaceutically acceptable salt is a cation salt selected from the group consisting of: a benzathine salt, a chloroprocaine salt, a choline salt, a tromethamine salt, a diethanolamine salt, an ethylenediamine salt, a meglumine salt, a procaine salt, an aluminum salt, a calcium salt, a lithium salt, a magnesium salt, a potassium salt, a sodium salt, and a zinc salt.

Examples of pharmaceutically acceptable salts also include those disclosed in e.g., Berge, Stephen M., Lyle D. Bighley, and Donald C. Monkhouse. “Pharmaceutical salts.” Journal of pharmaceutical sciences 66.1 (1977): 1-19 doi: 10.1002/jps.2600660104, Bharate, Sonali S. “Recent developments in pharmaceutical salts: FDA approvals from 2015 to 2019.” Drug Discovery Today 26.2 (2021): 384-398 doi: 10.1016/j.drudis.2020.11.016, and Bharate, Sonali S. “Modulation of biopharmaceutical properties of drugs using sulfonate counterions: A critical analysis of FDA-approved pharmaceutical salts.” Journal of Drug Delivery Science and Technology 66 (2021): 102913 doi: 10.1016/j.jddst.2021.102913.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

As used herein, “reacting” refers to bringing a chemical entity into physical contact with one or more additional chemical entities (reactants, reagents, catalysts, etc.) under appropriate conditions (e.g., appropriate temperature, appropriate solvent, appropriate reaction atmosphere, etc.) to facilitate one or more chemical transformations that lead to the product(s) as described herein.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Also provided herein are methods of preparing a compound of Formula (I-i1):

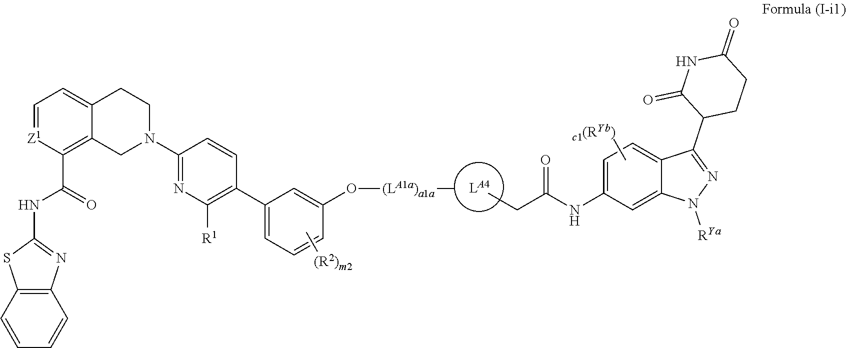

or a pharmaceutically acceptable salt thereof, comprising:
reacting a compound of Formula (SI-i1):

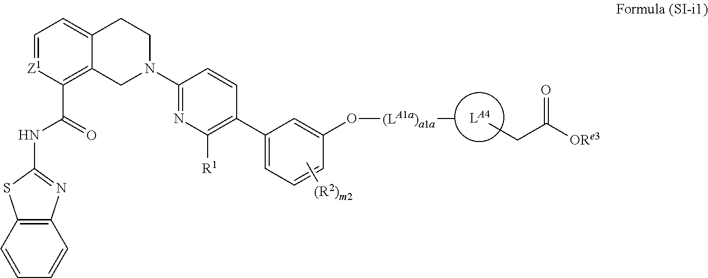

or a salt thereof, with a compound of Formula (SII-i1):

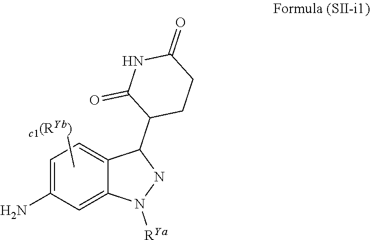

or a salt thereof, wherein:
Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a}.
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is

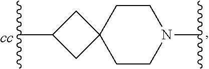

wherein cc represents point of attachment to -(L^{A1a})ₐ₁ₐ-;
R^{e3} is —H or C₁₋₆ alkyl;
R^{Ya} is selected from the group consisting of: —H, R^{b}, and C₁₋₆ alkyl optionally substituted with 1-3 R^{e};
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R9 is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (I-i1), Z¹, R¹, m2, R2, L^{A1a}ₐ₁ₐ L^{A1}, R^{L}, L^{A4}, R^{e3}, R^{Ya}, R^{Yb}, c1, R^{a}, R^{h}, b, L^{b}, R^{b1}, R^{c}, R^{d}, R^{e}, R^{f}, R^{g}, and R^{h} are each as defined herein.

In some embodiments of the methods of preparing a compound of Formula (I-i1), a1a is 0.

In some embodiments of the methods of preparing a compound of Formula (I-i1), R^{e}3 is —H.

In some embodiments of the methods of preparing a compound of Formula (I-i1), R^{e}3 is —H; and the methods comprise reacting a compound of Formula (SI-i1), or a salt thereof, with a compound of Formula (SII-i1), or a salt thereof, under standard conditions for amide bond formation to provide a compound of Formula (I-i1), or a salt thereof. Examples of standard conditions for amide bond formation include those described in Chem. Soc. Rev., 2009, 38, 606-631, which is incorporated herein by reference in its entirety.

In some embodiments of the methods of preparing a compound of Formula (I-i1), R^{e}3 is —H; and the methods comprise reacting a compound of Formula (SI-i1), or a salt thereof, with a compound of Formula (SII-i1), or a salt thereof, in the presence of a carboxyl activating agent (e.g., HATU, HOBT, EDCI, DCC, DIC, etc.) and optionally a base (e.g., triethylamine or diisopropylethylamine) to provide a compound of Formula (I-i1), or a salt thereof.

Also provided herein are methods of preparing a compound of Formula (I-f2):

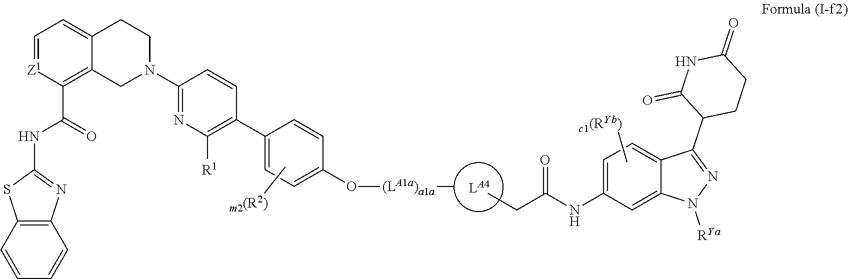

or a pharmaceutically acceptable salt thereof, comprising:
reacting a compound of Formula (SI-f2):

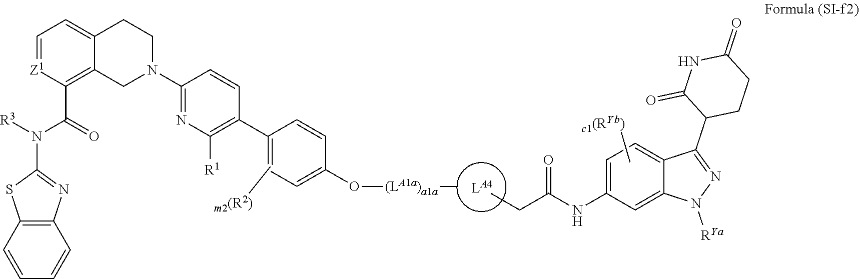

or a salt thereof, in the presence of a fluoride source (e.g., CsF) or an acid (e.g., TFA), wherein:
R³ is

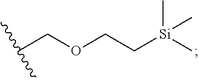 [SMILES: *COCC[Si](C)(C)C]

R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
m2 is 0 or 1;
R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{Ya} is selected from the group consisting of: —H, R^{b}, and C₁₋₆ alkyl optionally substituted with 1-3 R^{e};
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (I-2), or a salt thereof, the methods comprise preparing a compound of Formula (SI-f2), or a salt thereof, by reacting a compound of Formula (SI-f2-a):

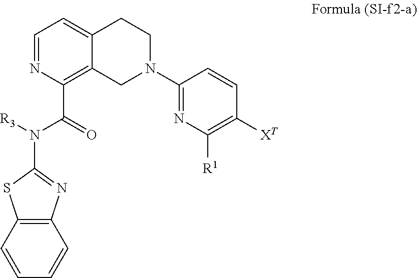

or a salt thereof, with a compound of Formula (SI-2-b):

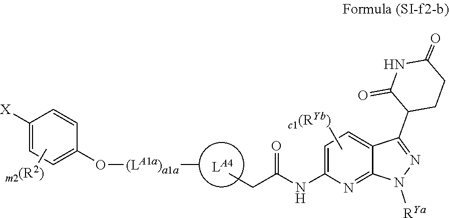

or a salt thereof, in the presence of a metal catalyst (e.g., a palladium catalyst such as cataCXium A Pd G₃), wherein:
R³ is

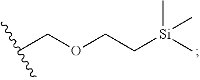 [SMILES: *COCC[Si](C)(C)C]

R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
X^{T} and X are each independently selected from the group consisting of: halo;
—B(OH)₂;
—BF₃⁻;
—B(OR^{T})₂, wherein each R^{T} is independently C₁₋₃ alkyl optionally substituted with 1-3 R^{c}; and

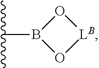

wherein L^{B} is C₂₋₆ alkylene optionally substituted with 1-4 R^{a};
m2 is 0 or 1;
R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{Ya} is selected from the group consisting of: —H, R^{b}, and C₁₋₆ alkyl optionally substituted with 1-3 R^{e};
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R)₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R9 is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (SI-f2), R³, R¹, X^{T}, X, R^{T}, L^{B}, m2, R², L^{A1a}, a1a, L^{A1}, R^{L}, L^{A4}, R^{L4}, R^{Ya}, R^{Yb}, c1, R^{a}, R^{h}, b, L^{b}, R^{b1}, R^{c}, R^{d}, R^{e}, R^{f}, R^{g}, and R^{h} are each as defined herein.

In some embodiments of the methods of preparing a compound of Formula (SI-f2), X^{T} is halo (e.g., Br).

In some embodiments of the methods of preparing a compound of Formula (SI-2), X

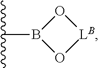

wherein L^{B} is C₂₋₆ alkylene optionally substituted with 1-4 R^{a} (e.g., R^{a} is C₁₋₆ alkyl).

In some embodiments of the methods of preparing a compound of Formula (SI-f2), X is

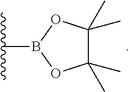 [SMILES: *B1OC(C)(C)C(C)(C)O1]

Also provided herein are methods of preparing a compound of Formula (I-h1):

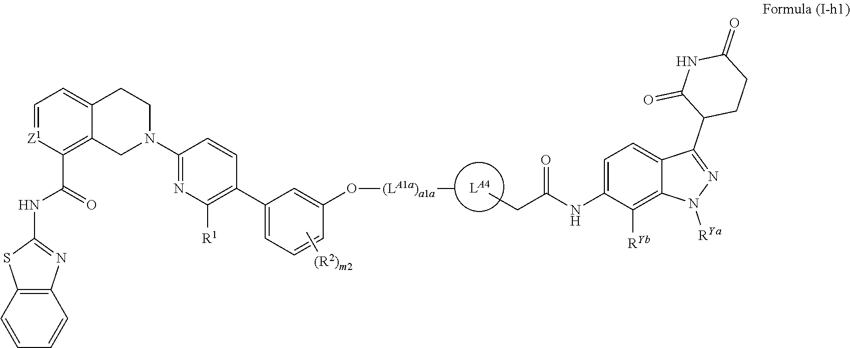

or a pharmaceutically acceptable salt thereof, comprising:
reacting a compound of Formula (SI-i1):

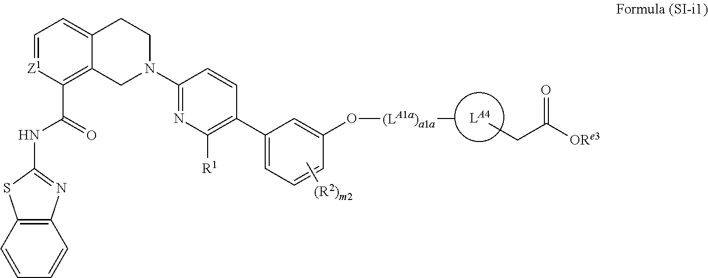

or a salt thereof, with a compound of Formula (SII-h1):

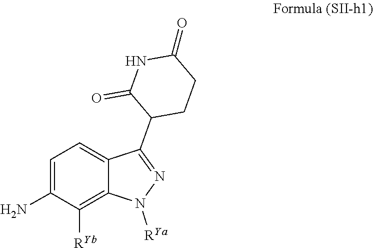

or a salt thereof, wherein:
Z¹ is selected from the group consisting of: N and CH;
R is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{e3} is —H or C₁₋₆ alkyl.
R^{Ya} is selected from the group consisting of: —H, R^{b}, and C₁₋₆ alkyl optionally substituted with 1-3 R^{c};
R^{Yb} is F, C₁₋₃ alkoxy, or C₁₋₃ alkyl optionally substituted with 1-3 —F.
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R)₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (I-h1), Z¹, R¹, m2, R2, L^{A1a}a1a L^{A1}, R^{L}, L^{A4}, RLA, R^{e3}, R^{Ya}, R^{Yb}, R^{a}, R^{b}, b, L^{b}, R^{b1}, R^{c}, R^{d}, R^{e}, R^{f}, R^{g}, and R^{h} are each as defined herein.

In some embodiments of the methods of preparing a compound of Formula (I-h1), R^{e}3 is —H.

In some embodiments of the methods of preparing a compound of Formula (I-h1), R^{Yb} is selected from the group consisting of: C₁₋₃ alkoxy, and C₁₋₃ alkyl optionally substituted with 1-3 F.

In some embodiments of the methods of preparing a compound of Formula (I-h1), R^{e3} is —H; and the methods comprise reacting a compound of Formula (SI-i1), or a salt thereof, with a compound of Formula (SII-h1), or a salt thereof, under standard conditions for amide bond formation to provide a compound of Formula (I-h1), or a salt thereof. Examples of standard conditions for amide bond formation include those described in Chem. Soc. Rev., 2009, 38, 606-631, which is incorporated herein by reference in its entirety.

In some embodiments of the methods of preparing a compound of Formula (I-h1), the methods comprise reacting a compound of Formula (SI-i1), or a salt thereof, with a compound of Formula (SII-h1), or a salt thereof, in the presence of a carboxyl activating agent (e.g., HATU, HOBT, EDCI, DCC, DIC, etc.) and optionally a base (e.g., triethylamine or diisopropylethylamine) to provide a compound of Formula (I-h1), or a salt thereof.

Also provided herein are methods of preparing a compound of Formula (I-h2):

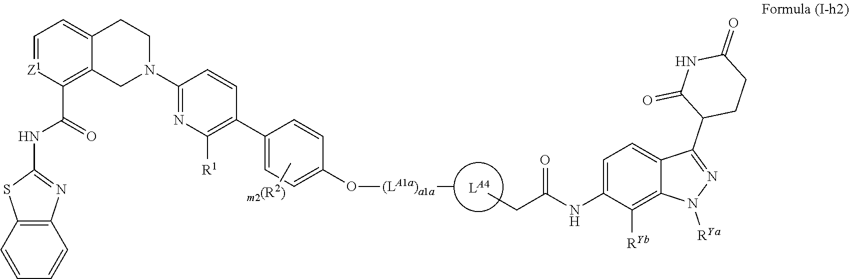

or a pharmaceutically acceptable salt thereof, comprising:
reacting a compound of Formula (SI-h2):

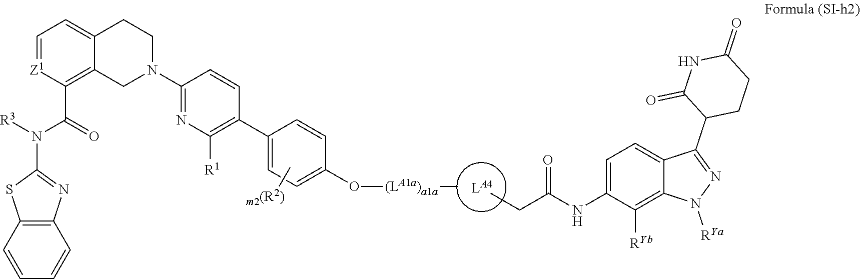

or a salt thereof, in the presence of a fluoride source (e.g., CsF) or an acid (e.g., TFA), wherein:
R³ is

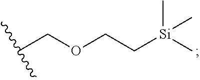 [SMILES: *COCC[Si](C)(C)C]

Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{Ya} is selected from the group consisting of: —H, R^{b}, and C₁₋₆ alkyl optionally substituted with 1-3 R^{c};
R^{Yb} is F, C₁₋₃ alkoxy, or C₁₋₃ alkyl optionally substituted with 1-3 —F;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R)₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀. 2(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R)₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (I-h2) or a salt thereof, comprises preparing a compound of Formula (SI-h2) or a salt thereof, by reacting a compound of Formula (SI-h2-a):

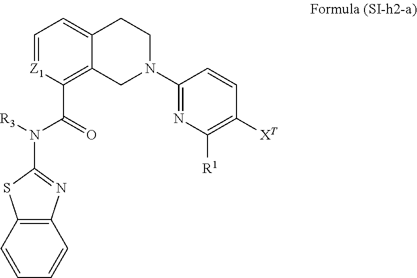

or a salt thereof, with a compound of Formula (SI-h2-b):

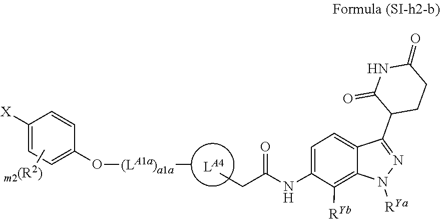

or a salt thereof, in the presence of a metal catalyst (e.g., a palladium catalyst such as cataCXium A Pd G₃), wherein:
R³ is

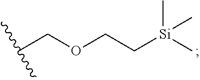 [SMILES: *COCC[Si](C)(C)C]

Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
X^{T} and X are each independently selected from the group consisting of:
halo;
—B(OH)₂;
—BF₃—;
—B(OR^{T})₂, wherein each R^{T} is independently C₁₋₃ alkyl optionally substituted with 1-3 R^{c}; and

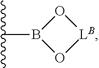

wherein L^{B} is C₂₋₆ alkylene optionally substituted with 1-4 R^{a};
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{Ya} is selected from the group consisting of: —H, R^{b}, and C₁₋₆ alkyl optionally substituted with 1-3 R^{c};
R^{Yb} is F, C₁₋₃ alkoxy, or C₁₋₃ alkyl optionally substituted with 1-3 —F;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R)₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (SI-h2), R3, Z¹, R¹, X^{T}, X, R^{T}, L^{B}, m2, R², L^{A1a}, a1a, L^{A1}, R^{L}, L^{A4}, R^{L4}, R^{Ya}, R^{Yb}, R^{a}, R, b, L^{b}, R^{b1}, R^{c}, R^{d}, R^{e}, R^{f}, R^{g}, and R^{h} are each as defined herein.

In some embodiments of the methods of preparing a compound of Formula (SI-h2), Z¹ is N.

In some embodiments of the methods of preparing a compound of Formula (SI-h2), R2 is CF₃.

In some embodiments of the methods of preparing a compound of Formula (SI-h2), X is halo (e.g., Br).

In some embodiments of the methods of preparing a compound of Formula (SI-h2), X^{T}

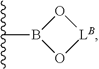

wherein L^{B} is C₂₋₆ alkylene optionally substituted with 1-4 R^{a} (e.g., R^{a} is C₁₋₆ alkyl).

In some embodiments of the methods of preparing a compound of Formula (SI-h2), X^{T} is

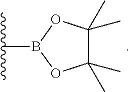 [SMILES: *B1OC(C)(C)C(C)(C)O1]

Also provided herein are methods of preparing a compound of Formula (I-i3):

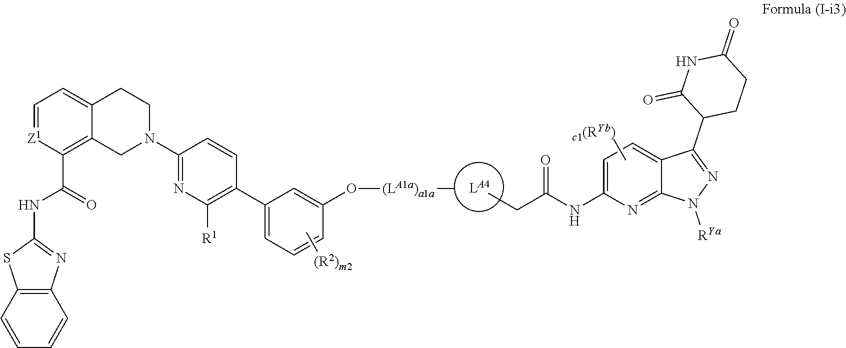

or a pharmaceutically acceptable salt thereof, comprising:
reacting a compound of Formula (SI-i3):

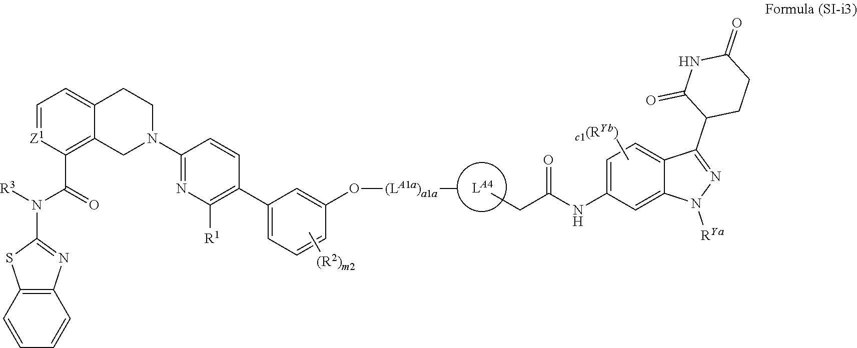

or a salt thereof, in the presence of a fluoride source (e.g., CsF) or an acid (e.g., TFA), wherein:
R³ is

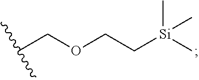 [SMILES: *COCC[Si](C)(C)C]

Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a}
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
   L^{A4} is

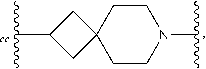

wherein cc represents point of attachment to -(L^{A1a})ₐ₁ₐ-;
R^{Ya} is selected from the group consisting of: —H, R^{b}, and C₁₋₆ alkyl optionally substituted with 1-3 R^{c};
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (I-i3) or a salt thereof, comprises preparing a compound of Formula (SI-i3) or a salt thereof, by reacting a compound of Formula (SI-i3-a):

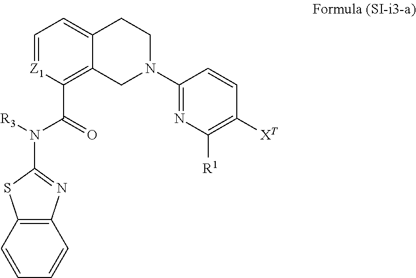

or a salt thereof, with a compound of Formula (SI-i3-b):

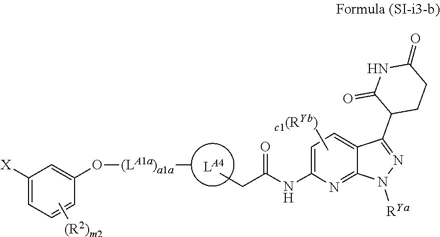

or a salt thereof, in the presence of a metal catalyst (e.g., a palladium catalyst such as cataCXium A Pd G₃), wherein:
R³ is

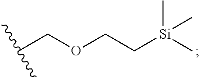 [SMILES: *COCC[Si](C)(C)C]

Z¹ is selected from the group consisting of: N and CH;
R¹ is selected from the group consisting of:
(a) —C(O)OH;
(b) —C(O)OC₁₋₆ alkyl, wherein the C₁₋₆ alkyl is optionally substituted with 1-3 R^{c}; and
(c) —C(O)—(C₀₋₃ alkylene)-phenyl, wherein the phenyl is optionally substituted with 1-3 R^{a};
X^{T} and X are each independently selected from the group consisting of:
halo;
—B(OH)₂;
—BF₃—;
—B(OR^{T})₂, wherein each R^{T} is independently C₁₋₃ alkyl optionally substituted with 1-3 R^{c}; and

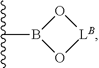

wherein L^{B} is C₂₋₆ alkylene optionally substituted with 1-4 R^{a};
m2 is 0 or 1;
   R² is CH₃ or CF₃;
L^{A1a} is an independently selected L^{A1};
   a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, C₁₋₆ alkoxy, C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
   L^{A4} is

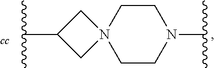

wherein cc represents point of attachment to -(L^{A1a})ₐ₁ₐ-;
R^{Ya} is selected from the group consisting of: —H, R^{b}, and C₁₋₆ alkyl optionally substituted with 1-3 R^{c};
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and R^{b1}, wherein:
each b is independently 1 or 2;
each L^{b} is independently selected from the group consisting of: —O—, —N(H)—, —N(C₁₋₃ alkyl)-, —S(O)₀₋₂—, —C(═O)—, and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, —CN, —OH, C₁₋₆ alkoxy, C₁₋₆ haloalkoxy, —NR^{d}R^{e}, —C(═O)C₁₋₆ alkyl, —C(═O)OC₁₋₆ alkyl, —C(═O)N(R^{f})₂, —S(O)₀₋₂(C₁₋₆ alkyl), and —S(O)₀₋₂(C₁₋₆ haloalkyl);
each R^{d} and R^{e} is independently selected from the group consisting of: —H, —C(═O)C₁₋₆ alkyl, —C(═O)C₁₋₆ haloalkyl, —C(═O)OC₁₋₆ alkyl, —C(═O)OC₁₋₆ haloalkyl, —C(═O)N(R^{f})₂, —S(O)₁₋₂(C₁₋₆ alkyl), —S(O)₁₋₂(C₁₋₆ haloalkyl), —S(O)₁₋₂N(R^{f})₂, and C₁₋₆ alkyl optionally substituted with 1-3 R^{h};
each R^{f} is independently selected from the group consisting of: —H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, —CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of the methods of preparing a compound of Formula (SI-i3), R3, Z¹, R¹, X^{T}, X, R^{T}, L^{B}, m2, R2, L^{A1a}, a1a L^{A1}, R^{L}, L^{A4}, R^{Ya}, R^{Yb}, c1, R^{a}, R^{b}, b, L^{b}, R^{b1}, R^{c}, R^{d}, R^{e}, R^{f}, R^{g}, and R^{h} are each as defined herein.

In some embodiments of the methods of preparing a compound of Formula (SI-i3), Z¹ is N.

In some embodiments of the methods of preparing a compound of Formula (SI-i3), R2 is CH₃.

In some embodiments of the methods of preparing a compound of Formula (SI-i3), a1a is 0.

In some embodiments of the methods of preparing a compound of Formula (SI-i3), X is halo (e.g., Br).

In some embodiments of the methods of preparing a compound of Formula (SI-i3), X^{T} is

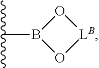

wherein L^{B} is C₂₋₆ alkylene optionally substituted with 1-4 R^{a} (e.g., R^{a} is C₁₋₆ alkyl).

In some embodiments of the methods of preparing a compound of Formula (SI-i3), X^{T} is

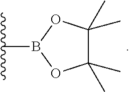 [SMILES: *B1OC(C)(C)C(C)(C)O1]

Also provided herein are compounds of Formula (SI-i1-a):

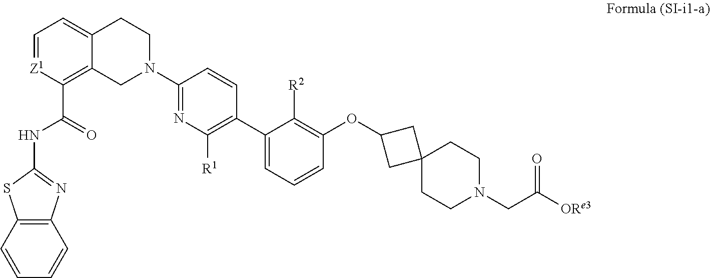

or salts thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{a};
R² is CH₃ or CF₃;
R^{e3} is —H or C₁₋₆ alkyl;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h}; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SI-i1-a), Z¹ is CH.

In some embodiments of Formula (SI-i1-a), R³ is H.

In some embodiments of Formula (SI-i1-a), R^{b1} is C(O)OC₁₋₆ alkyl, wherein the C₁₋₆ alkyl is optionally substituted with 1-3 R^{c}.

In some embodiments of Formula (SI-i1-a), R¹ is C(O)OC₁₋₆ alkyl.

In some embodiments of Formula (SI-i1-a), R^{b1} is C(O)Ot-Bu.

For example, a compound of Formula (SI-i1-a) is

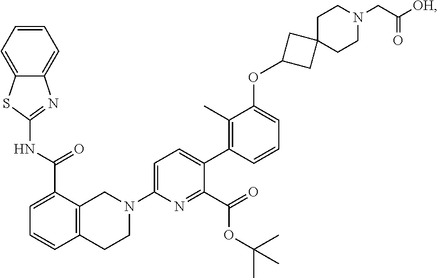 [SMILES: Cc1c(OC2CC3(CCN(CC(=O)O)CC3)C2)cccc1-c1ccc(N2CCc3cccc(C(=O)Nc4nc5ccccc5s4)c3C2)nc1C(=O)OC(C)(C)C]

or a salt thereof.

Also provided herein are compounds of Formula (SI-f2-a):

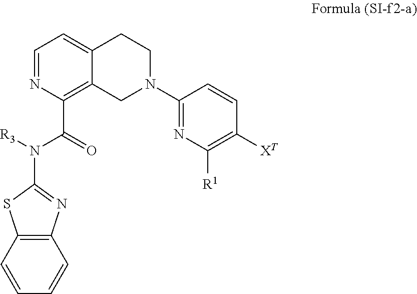

or salts thereof, wherein:
R³ is —H or

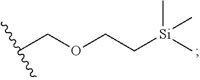 [SMILES: *COCC[Si](C)(C)C]

R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{a};
X^{T} is selected from the group consisting of:
halo;
—B(OH)₂;
—BF₃—;
—B(OR^{T})₂, wherein each R^{T} is independently C₁₋₃ alkyl optionally substituted with 1-3 R^{c}; and

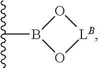

wherein L^{B} is C₂₋₆ alkylene optionally substituted with 1-4 R^{a};
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h}; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SI-f2-a), R¹ is C(O)OC₁₋₆ alkyl, wherein the C₁₋₆ alkyl is optionally substituted with 1-3 R^{c}.

In some embodiments of Formula (SI-2-a), R is C(O)OC₁₋₆ alkyl.

In some embodiments of Formula (SI-2-a), R^{b1} is C(O)Ot-Bu.

In some embodiments of Formula (SI-2-a), X^{T} is halo (e.g., Br).

In some embodiments of Formula (SI-2-a), X^{T} is —B(OR^{T})₂, wherein each R^{T} is independently C₁₋₃ alkyl optionally substituted with 1-3 R^{c}.

In some embodiments of Formula (SI-f2-a), X^{T} is

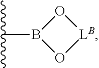

wherein L^{B} is C₂₋₆ alkylene optionally substituted with 1-4 R^{a} (e.g., R^{a} is C₁₋₆ alkyl). For example, X^{T} can be

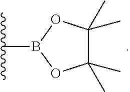 [SMILES: *B1OC(C)(C)C(C)(C)O1]

For example, a compound of Formula (SI-f2-a) can be:

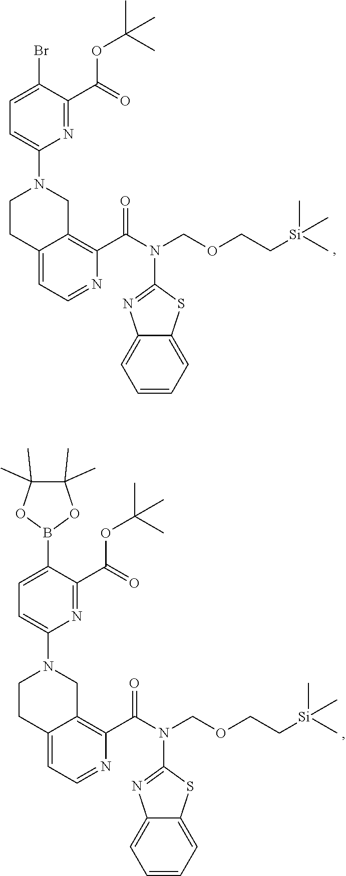

or salts thereof.

Also provided herein are compounds of Formula (SI-f2-b):

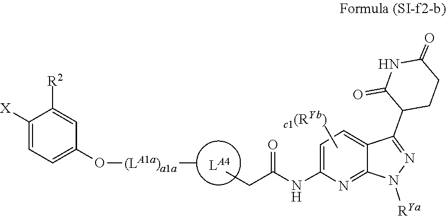

or salts thereof, wherein:
X is selected from the group consisting of:
halo;
—B(OH)₂;
—BF₃—;
—B(OR^{T})₂, wherein each R^{T} is independently C₁₋₃ alkyl optionally substituted with 1-3 R^{c}; and

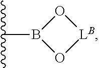

wherein L^{B} is C₂₋₆ alkylene optionally substituted with 1-4 R^{a};
R² is CH₃ or CF₃;
L^{A1a} is an independently selected L^{A1};
each L^{A1} is independently selected from the group consisting of: —CH₂—, —CHR^{L}—, and —C(R^{L})₂—, wherein: each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c}; a1a is an integer from 0 to 4;
L^{A4} is a 5-12 membered nitrogen-containing heterocyclylene optionally substituted with 1-3 R^{L4}; wherein each R^{L4} is independently selected from the group consisting of R^{a} and R^{b};
R^{Ya} is selected from the group consisting of: H, R^{b}, and C₁₋₆ alkyl optionally substituted with 1-3 R^{e},
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SI-f2-b), X is —B(OR^{T})₂, wherein each R^{T} is independently C₁₋₃ alkyl optionally substituted with 1-3 R^{c}.

In some embodiments of Formula (SI-f2-b), X is

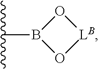

wherein L^{B} is C₂₋₆ alkylene optionally substituted with 1-4 R^{a} (e.g., R^{a} is C₁₋₆ alkyl). For example, X can be

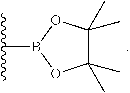 [SMILES: *B1OC(C)(C)C(C)(C)O1]

In some embodiments of Formula (SI-2-b), R² is —CH₃.

In some embodiments of Formula (SI-2-b), each R^{L} is independently selected C₁₋₃ alkyl optionally substituted with 1-3 —F.

In some embodiments of Formula (SI-2-b), one L^{A1a} is —CHR^{L}— (e.g., —CHMe-); and each remaining L^{A1a} is —CH₂—.

In some embodiments of Formula (SI-2-b), each L^{A1a} is —CH₂—.

In some embodiments of Formula (SI-2-b), a1a is 3 or 4.

In some embodiments of Formula (SI-2-b), c1 is 0.

In some embodiments of Formula (SI-2-b), R^{Ya} is C₁₋₃ alkyl (e.g., methyl).

For example, a compound of Formula (SI-f2-b), is

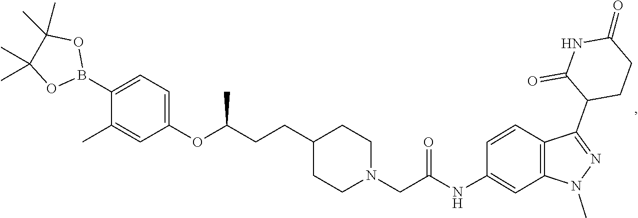 [SMILES: Cc1cc(O[C@@H](C)CCC2CCN(CC(=O)Nc3ccc4c(C5CCC(=O)NC5=O)nn(C)c4c3)CC2)ccc1B1OC(C)(C)C(C)(C)O1]

or a salt thereof.

Also provided herein are compounds of Formula (SII-h1):

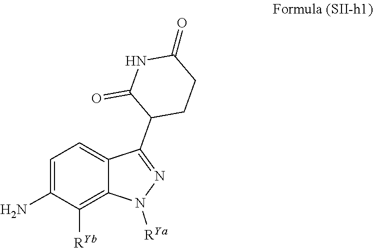

or salts thereof, wherein:
R^{Ya} is selected from the group consisting of: H, R^{b}, and C₁₋₆ alkyl optionally substituted with 1-3 R^{c}; and
R^{Yb} is C₁₋₃ alkoxy, or C₁₋₃ alkyl optionally substituted with 1-3 —F.
each R^{b} is independently selected from the group consisting of: -(L^{b})_{b}-R^{b1} and —R^{h}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SII-h1), R^{Y}b is C₁₋₃ alkoxy.

In some embodiments of Formula (SII-h1), R^{Y}b is —OCH₃.

For example, a compound of Formula (SII-h1) is

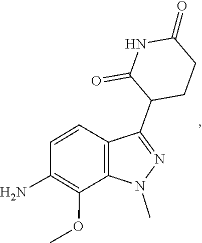 [SMILES: COc1c(N)ccc2c(C3CCC(=O)NC3=O)nn(C)c12]

or a salt thereof.

Also provided herein are compounds of Formula (SI-h2-b):

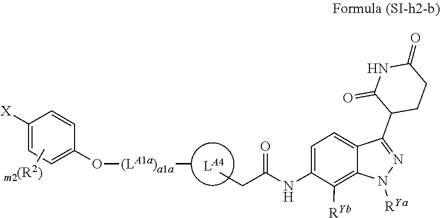

or salts thereof, wherein:
X is selected from the group consisting of:
halo;
—B(OH)₂;
—BF₃—;
—B(OR^{T})₂, wherein each R^{T} is independently C₁₋₃ alkyl optionally substituted with 1-3 R^{c}; and

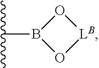

wherein L^{B} is C₂₋₆ alkylene optionally substituted with 1-4 R^{a};
m2 is 0 or 1;
R² is CH₃ or CF₃;
L^{A1a} is an independently selected L^{A1};
a1a is an integer from 0 to 4;
each L^{A1} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
L^{A4} is a 5-12 membered nitrogen-containing heterocyclylene optionally substituted with 1-3 R^{L4};
wherein each R^{L4} is independently selected from the group consisting of R^{a} and R^{b};
R^{Ya} is selected from the group consisting of: H, R^{b}, and C₁₋₆ alkyl optionally substituted with 1-3 R^{c};
R^{Yb} is F, C₁₋₃ alkoxy, or C₁₋₃ alkyl optionally substituted with 1-3 —F;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e}
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{h}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h},
each R9 is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SI-h2-b), X is halo (e.g., Br).

In some embodiments of Formula (SI-h2-b), m2 is 1.

In some embodiments of Formula (SI-h2-b), R² is —CF₃.

In some embodiments of Formula (SI-h2-b), each R^{L} is independently selected C₁₋₃ alkyl optionally substituted with 1-3 —F.

In some embodiments of Formula (SI-h2-b), one L^{A1a} is —CHR^{L}— (e.g., —CHMe-); and
each remaining L^{A1a} is —CH₂—.

In some embodiments of Formula (SI-h2-b), each L^{A1a} is —CH₂—.

In some embodiments of Formula (SI-h2-b), a1a is 1.

In some embodiments of Formula (SI-h2-b), L^{A4}, is

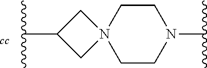

wherein cc represents point of attachment to -(L^{A1a})ₐ₁ₐ-.

In some embodiments of Formula (SI-h2-b), R^{Ya} is C₁₋₃ alkyl (e.g., methyl).

In some embodiments of Formula (SI-h2-b), R^{Yb} is C₁₋₃ alkoxy.

In some embodiments of Formula (SI-h2-b), R^{Yb} is —OCH₃.

For example, a compound of Formula (SI-h2-b) is

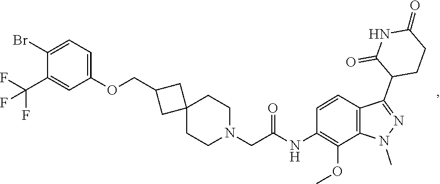 [SMILES: COc1c(NC(=O)CN2CCC3(CC2)CC(COc2ccc(Br)c(C(F)(F)F)c2)C3)ccc2c(C3CCC(=O)NC3=O)nn(C)c12]

or a salt thereof.

Also provided herein are compounds of Formula (SI-i3-b):

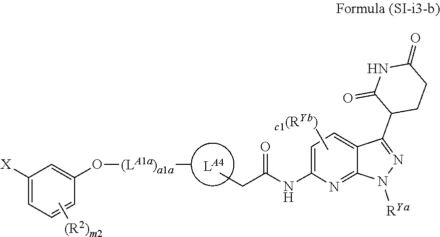

or salts thereof, wherein:
X is selected from the group consisting of:
halo;
—B(OH)₂;
—BF₃—;
—B(OR^{T})₂, wherein each R^{T} is independently C₁₋₃ alkyl optionally substituted with 1-3 R^{c}; and

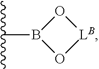

wherein L^{B} is C₂₋₆ alkylene optionally substituted with 1-4 R^{a};
m2 is 0 or 1;
R² is CH₃ or CF₃;
L^{A1a} is an independently selected L^{A1};
a1a is an integer from 0 to 4;
each L^{A1} is independently selected from the group consisting of: —CH₂—, —CHR^{L}—, and —C(R^{L})₂—, wherein: each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
   L^{A4} is

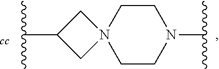

wherein cc represents point of attachment to -(L^{A1a})ₐ₁ₐ-;
R^{Ya} is selected from the group consisting of: H, R^{b}, and C₁₋₆ alkyl optionally substituted with 1-3 R^{e},
each R^{Yb} is independently selected from the group consisting of R^{a} and R^{b};
c1 is 0, 1, or 2;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{h}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂ (C₁₋₆ alkyl), S(O)₁₋₂(C₁₋₆ haloalkyl), S(O)₁₋₂N(R^{f})₂, and C₁₋₆ alkyl optionally substituted with 1-3 R^{h}.
each R^{f} is independently selected from the group consisting of: H and C₁₋₆ alkyl optionally substituted with 1-3 R^{h};
each R9 is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments of Formula (SI-i3-b), X is halo (e.g., Br).

In some embodiments of Formula (SI-i3-b), m2 is 1.

In some embodiments of Formula (SI-i3-b), R² is —CH₃.

In some embodiments of Formula (SI-i3-b), a1a is 0.

In some embodiments of Formula (SI-i3-b), c1 is 0.

In some embodiments of Formula (SI-i3-b), R^{Ya} is C₁₋₃ alkyl (e.g., methyl).

For example, a compound of Formula (SI-i3-b) is

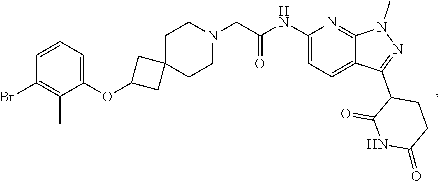 [SMILES: Cc1c(Br)cccc1OC1CC2(CCN(CC(=O)Nc3ccc4c(C5CCC(=O)NC5=O)nn(C)c4n3)CC2)C1]

or a salt thereof.

### Examples

In some of the examples disclosed herein, the final product of a described chemical reaction sequence is structurally depicted with an enhanced stereochemical or 1 notation at one stereogenic center. In some such examples, in the chemical name of the same compound, this stereogenic center is assigned a tentative configuration (e.g., (R)- or (S)-) based on the wedge/dash representation of the structural formula. However, this stereogenic center should be understood to have a configuration consistent with the or 1 notation. Specifically, this stereogenic center has been resolved, but its specific configuration has not been determined. Accordingly, unless otherwise specified, starting materials and intermediates leading to this compound incorporate the or 1 notation at this stereogenic center following stereochemical resolution(s), notwithstanding the tentative assignments provided in their chemical names.

For example, Compound 130a in Example 9 is a single stereoisomer selected from:

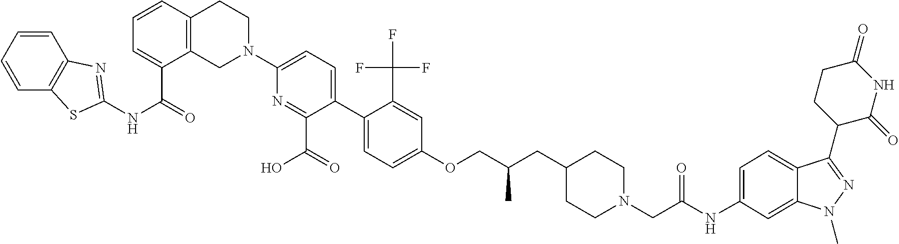 [SMILES: C[C@@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1]

6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid; and

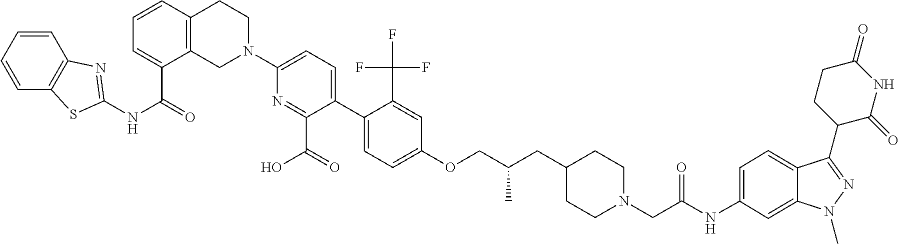 [SMILES: C[C@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1]

6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid.

The intermediate product provided in Step 2 of the same example incorporates the or 1 notation. It is therefore a single stereoisomer selected from:

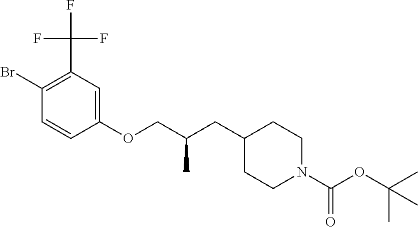 [SMILES: C[C@@H](COc1ccc(Br)c(C(F)(F)F)c1)CC1CCN(C(=O)OC(C)(C)C)CC1]

tert-butyl (R)-4-(3-(4-bromo-3-(trifluoromethyl)phenoxy)-2-methylpropyl)piperidine-1-carboxylate; and

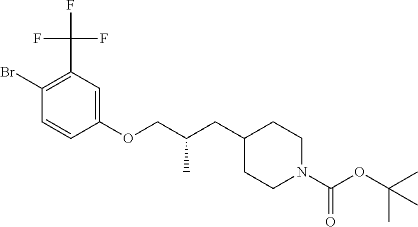 [SMILES: C[C@H](COc1ccc(Br)c(C(F)(F)F)c1)CC1CCN(C(=O)OC(C)(C)C)CC1]

tert-butyl (S)-4-(3-(4-bromo-3-(trifluoromethyl)phenoxy)-2-methylpropyl)piperidine-1-carboxylate.

For further clarification, a chemical name that takes into account the or 1 notation is provided for the final product having an enhanced stereochemical or 1 notation at one stereogenic center. This chemical name is enclosed in brackets (i.e., “[ ]”). In these chemical names, the prefix “ref” means that the stereochemical configuration shown in a chemical name is relative. To illustrate, when a compound contains one stereogenic center, and its chemical name starts with the prefix “rel,” then this stereogenic center is resolved, but its absolute configuration is either (R)- or (S)—. As such, it should be labelled with an or 1 enhanced stereochemical notation in its corresponding structure.

For example, the chemical name that takes into account the or 1 notation for Compound 130a is:
[rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid], as included in Example 9.

### Example P1: tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate

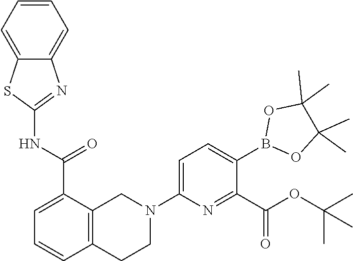 [SMILES: CC(C)(C)OC(=O)c1nc(N2CCc3cccc(C(=O)Nc4nc5ccccc5s4)c3C2)ccc1B1OC(C)(C)C(C)(C)O1]

### Step 1: 2-(tert-butyl) 8-methyl 3,4-dihydroisoquinoline-2,8(1H)-dicarboxylate

To a solution of 2-tert-butoxycarbonyl-3,4-dihydro-1H-isoquinoline-8-carboxylic acid (62 g, 223.57 mmol, 1 equiv.) in DMF (600 mL) was added Mel (38.08 g, 268.29 mmol, 16.70 mL, 1.2 equiv.) and potassium carbonate (61.80 g, 447.15 mmol, 2 equiv.). The mixture was stirred at 50° C. for 2 hours. The mixture was poured into ice water (800 mL) (pH>8), extracted with EtOAc (900 mL). The organic layers were combined and washed with brine (900 mL), dried over sodium sulfate, and concentrated in vacuo to afford 2-(tert-butyl) 8-methyl 3,4-dihydroisoquinoline-2,8(1H)-dicarboxylate (66 g, crude) as a brown oil which was used in the next step without further purification.

### Step 2: tert-butyl 8-(1,3-benzothiazol-2-ylcarbamoyl)-3,4-dihydro-1H-isoquinoline-2-carboxylate

To a solution of 1,3-benzothiazol-2-amine (35.73 g, 237.87 mmol, 1.05 equiv.) in 2-MeTHF (350 mL) was added LiHMDS (1 M, 453.08 mL, 2 equiv.) at 0° C., and the solution was stirred at 0° C. for 30 minutes. Then, a solution of 2-(tert-butyl) 8-methyl 3,4-dihydroisoquinoline-2,8(1H)-dicarboxylate (66.87 g, 226.54 mmol, 98.7% purity, 1 equiv.) in 2-MeTHF (350 mL) was added at 0° C. The resulting mixture was warmed to 60° C. and stirred for 17 hours. The reaction mixture was poured into saturated ammonium chloride (1 L) and extracted with EtOAc (1500 mL). The combined organic layer was washed with brine, dried over sodium sulfate, and concentrated in vacuo to give a crude product which was purified by re-crystallization from EtOAc (500 mL) at 25° C. The filtrate was concentrated in vacuo to give a residue. The residue was dissolved EtOAc (300 mL), the solution was washed with 10% citric acid (300 mL) and brine (150 mL), dried over sodium sulfate, and concentrated under reduced pressure to give a residue which was purified by re-crystallization from ethyl acetate/petroleum ether=1/1 (600 ml) at 25° C. The second recrystallized mother liquor was concentrated in vacuo to give a residue which was purified by flash silica gel chromatography (0~40% DCM/petroleum ether) to give tert-butyl 8-(1,3-benzothiazol-2-ylcarbamoyl)-3,4-dihydro-1H-isoquinoline-2-carboxylate (66.7 g) as a light yellow solid.

LC-MS (ESI): m/z=410.0 [M+H]⁺.

¹H NMR (400 MHz, CDCl₃) δ=7.87-7.79 (m, 1H), 7.51 (d, J=7.4 Hz, 1H), 7.33-7.26 (m, 3H), 7.22 (d, J=7.4 Hz, 1H), 7.17-7.10 (m, 1H), 4.91 (s, 2H), 3.64 (s, 2H), 2.88 (s, 2H), 1.57-1.34 (m, 9H)

### Step 3: N-(1,3-benzothiazol-2-yl)-1,2,3,4-tetrahydroisoquinoline-8-carboxamide

To a mixture of tert-butyl 8-(1,3-benzothiazol-2-ylcarbamoyl)-3,4-dihydro-1H-isoquinoline-2-carboxylate (61.7 g, 150.67 mmol, 1 equiv.) in DCM (1000 mL) was added HCl/dioxane (600 mL). The mixture was stirred at 25° C. for 6 hours. The reaction mixture was concentrated in vacuo to give N-(1,3-benzothiazol-2-yl)-1,2,3,4-tetrahydroisoquinoline-8-carboxamide (59 g, crude, 2HCl) as a white solid. The solid was used in the next step without further purification.

¹H NMR (400 MHz, DMSO-d₆) δ=9.63 (br s, 2H), 8.02 (d, J=7.8 Hz, 1H), 7.87-7.61 (m, 2H), 7.55-7.22 (m, 4H), 4.47-4.36 (m, 2H), 3.56 (s, 3H), 3.43-3.28 (m, 2H), 3.11 (t, J=5.8 Hz, 2H)

### Step 4: tert-butyl 6-[8-(1,3-benzothiazol-2-ylcarbamoyl)-3,4-dihydro-1H-isoquinolin-2-yl]-3-bromo-pyridine-2-carboxylate

To a mixture of N-(1,3-benzothiazol-2-yl)-1,2,3,4-tetrahydroisoquinoline-8-carboxamide (30 g, 73.76 mmol, 1 equiv., 2HCl) in DMA (300 mL) was added Cs₂CO₃ (120.17 g, 368.81 mmol, 5 equiv.) and tert-butyl 3-bromo-6-chloro-pyridine-2-carboxylate (21.80 g, 74.50 mmol, 1.01 equiv.). The mixture was stirred at 120° C. for 12 hours followed by the addition of tert-butyl 3-bromo-6-chloro-pyridine-2-carboxylate (5.8 g, 19.83 mmol, 0.269 equiv.). The resulting mixture was stirred at 120° C. for 7 hours. The reaction mixture was cooled to room temperature and poured into ice water (1 L). The mixture was stirred for 10 minutes and filtered. The cake was washed with EtOH and collected. The cake was triturated with EtOH (200 mL) at 25° C. for 30 minutes to give tert-butyl 6-[8-(1,3-benzothiazol-2-ylcarbamoyl)-3,4-dihydro-1H-isoquinolin-2-yl]-3-bromo-pyridine-2-carboxylate (24.6 g) as a white solid.

LC-MS (ESI): m/z=565.0 [M+H]⁺

¹H NMR (400 MHz, DMSO-d₆) δ=12.70 (brs, 1H), 8.01 (d, J=7.8 Hz, 1H), 7.77 (dd, J=2.8, 8.4 Hz, 2H), 7.58 (d, J=7.4 Hz, 1H), 7.50-7.39 (m, 2H), 7.39-7.30 (m, 2H), 6.86 (d, J=9.2 Hz, 1H), 4.93 (s, 2H), 3.77 (t, J=6.0 Hz, 2H), 3.00 (t, J=5.8 Hz, 2H), 1.34 (s, 9H)

### Step 5: tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate

A mixture of tert-butyl 6-[8-(1,3-benzothiazol-2-ylcarbamoyl)-3,4-dihydro-1H-isoquinolin-2-yl]-3-bromo-pyridine-2-carboxylate (1.5 g, 2.65 mmol, 1 equiv.), 4,4,5,5-tetramethyl-1,3,2-dioxaborolane (1.70 g, 13.26 mmol, 1.92 mL, 5 equiv.), DIPEA (805.25 mg, 7.96 mmol, 1.11 mL, 3 equiv.), and Pd(dppf)Cl₂ (194.09 mg, 265.26 μmol, 0.1 equiv.) in THF (3 mL) and CH₃CN (10 mL) was degassed and purged with N₂ three times, and then the mixture was stirred at 100° C. for 1 hour under N₂ atmosphere in microwave. The reaction was filtered to give tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (2 g) as a white solid.

LC-MS (ESI): m/z=613.7 [M+H]⁺.

¹H NMR (400 MHz, DMSO-d₆) δ=12.84 (s, 1H), 8.03 (d, J=7.6 Hz, 1H), 7.78 (d, J=8.2 Hz, 1H), 7.68 (d, J=8.6 Hz, 1H), 7.56 (d, J=7.2 Hz, 1H), 7.50-7.39 (m, 2H), 7.38-7.30 (m, 2H), 6.86 (d, J=8.8 Hz, 1H), 5.01 (s, 2H), 3.82 (t, J=5.8 Hz, 2H), 3.00 (t, J=5.8 Hz, 2H), 1.28 (s, 9H), 1.22 (s, 12H)

### Example 1: 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid (Compound 104)

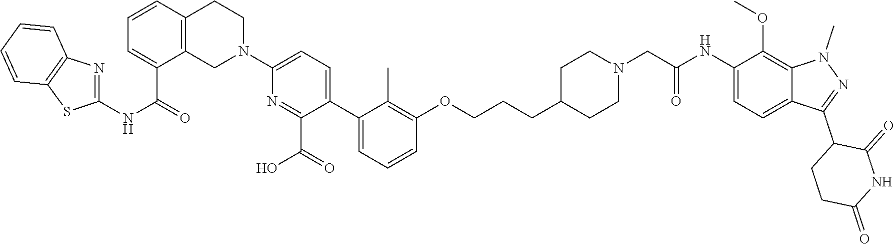 [SMILES: COc1c(NC(=O)CN2CCC(CCCOc3cccc(-c4ccc(N5CCc6cccc(C(=O)Nc7nc8ccccc8s7)c6C5)nc4C(=O)O)c3C)CC2)ccc2c(C3CCC(=O)NC3=O)nn(C)c12]

### Step 1: tert-Butyl 4-(3-(3-bromo-2-methylphenoxy)propyl)piperidine-1-carboxylate

A mixture containing 3-bromo-2-methylphenol (1 equiv.), tert-butyl 4-(3-bromopropyl)piperidine-1-carboxylate (1 equiv.), and potassium carbonate (3 equiv.) in DMF was heated at 50° C. for 16 hours. The mixture was diluted with ethyl acetate and brine. The layers were separated and the organic layers were combined and dried over anhydrous magnesium sulfate, filtered, and concentrated to an oil that was purified by silica gel chromatography (heptane/ethyl acetate gradient) to afford tert-butyl 4-(3-(3-bromo-2-methylphenoxy)propyl)piperidine-1-carboxylate as an oil that was used without further purification.

LC-MS (ESI): m/z=412.2 [M+H]⁺.

### Step 2: 4-(3-(3-Bromo-2-methylphenoxy)propyl)piperidine, HCl

A mixture containing tert-butyl 4-(3-(3-bromo-2-methylphenoxy)propyl)piperidine-1-carboxylate (1 equiv.) and 4 M HCl in dioxane (10 equiv.) in DCM was stirred under ambient temperature for 40 hours. The mixture was concentrated under reduced pressure, triturated with diethyl ether and hexanes, re-evaporated, and dried the resulting solids overnight under vacuum to afford 4-(3-(3-bromo-2-methylphenoxy)propyl)piperidine, HCl.

LC-MS (ESI): m/z=312.2 [M+H]⁺.

### Step 3: Methyl 2-(4-(3-(3-bromo-2-methylphenoxy)propyl)piperidin-1-yl)acetate

A mixture containing 4-(3-(3-bromo-2-methylphenoxy)propyl)piperidine, HCl (1 equiv.), methyl 2-bromoacetate (1 equiv.), and potassium carbonate (4 equiv.) in DMF was heated overnight at 50° C. for 16 hours. The mixture was diluted with ethyl acetate and brine. The mixture was then separated and the organic layers were combined and dried over anhydrous magnesium sulfate, filtered, and concentrated to an oil that was purified by silica gel chromatography (heptane/ethyl acetate (w/10% of a 7N NH₃ solution in MeOH)) to afford methyl 2-(4-(3-(3-bromo-2-methylphenoxy)propyl)piperidin-1-yl)acetate.

LC-MS (ESI): m/z=384.2 [M+H]⁺.

### Step 4: Methyl 2-(4-(3-(2-methyl-3-(4,4,5,5-tetramethyl-1,3,2-dioxaborolan-2-yl)phenoxy)propyl)piperidin-1-yl)acetate

Methyl 2-(4-(3-(3-bromo-2-methylphenoxy)propyl)piperidin-1-yl)acetate (1 equiv.), bis(pinacolato)diboron (1.7 equiv.), and PdCl₂(dppf)-CH₂Cl₂ adduct (0.1 equiv.) were combined in dioxane. Potassium acetate (3 equiv.) was then added, and the resulting mixture was stirred at 80° C. for 16 hours. The mixture was diluted with ethyl acetate, water, and brine. The layers were separated and the organic layer was washed with brine. The organic layer was dried over anhydrous magnesium sulfate, filtered, and concentrated to dryness under reduced pressure. The residue was purified by silica gel chromatography (heptane/ethyl acetate gradient) to afford methyl 2-(4-(3-(2-methyl-3-(4,4,5,5-tetramethyl-1,3,2-dioxaborolan-2-yl)phenoxy)propyl)piperidin-1-yl)acetate which was used without further purification.

LC-MS (ESI): m/z=432.4 [M+H]⁺.

### Step 5: tert-Butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate

To a mixture containing tert-butyl 6-(8-(benzo[d]thiazol-2-ylcarbamoyl)-3,4-dihydroisoquinolin-2(1H)-yl)-3-bromopicolinate and methyl 2-(4-(3-(2-methyl-3-(4,4,5,5-tetramethyl-1,3,2-dioxaborolan-2-yl)phenoxy)propyl)piperidin-1-yl)acetate in dioxane was added potassium phosphate (4 equiv.) and Ad₂nBup Pd G₃ (0.05 equiv.). The mixture was stirred at 100° C. for 1 hour in a microwave reactor. The reaction mixture was diluted with ethyl acetate, water, and brine. The organic layers were combined and washed with brine. The organic layer was dried over anhydrous magnesium sulfate, filtered, and concentrated to dryness under reduced pressure to afford tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate which was used without further purification.

LC-MS (ESI): m/z=790.4 [M+H]⁺.

### Step 6: 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

tert-Butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate (1 equiv.) was dissolved in a mixture of THF, MeOH, and water. Lithium hydroxide (10 equiv.) was added, and the mixture was stirred under ambient temperature for 16 hours. The pH was adjusted to pH ~5-6 with formic acid and the volatiles were removed under reduced pressure to provide solids that were filtered, rinsed with water, and dried under vacuum to afford 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid which was used without further purification.

LC-MS (ESI): m/z=776.4 [M+H]⁺.

### Step 7: (E)-1-(4-bromo-2-fluoro-3-methoxy-phenyl)-N-methoxy-methanimine

To a solution of 4-bromo-2-fluoro-3-methoxy-benzaldehyde (19 g, 81.53 mmol, 1 equiv.) in DME (250 mL) was added potassium carbonate (33.80 g, 244.60 mmol, 3 equiv.) and O-methylhydroxylamine hydrochloride (7.49 g, 89.69 mmol, 6.81 mL, 1.1 equiv.). The mixture was stirred at 40° C. for 2.5 hours. The reaction mixture was concentrated under reduced pressure to remove solvent to give a residue, which was purified by flash silica gel chromatography (0-5% ethyl acetate/petroleum ether) to afford (E)-1-(4-bromo-2-fluoro-3-methoxy-phenyl)-N-methoxy-methanimine (21 g) as a white solid.

¹H NMR (400 MHz, DMSO-d₆) δ=8.27 (s, 1H), 7.54-7.47 (m, 1H), 7.44-7.36 (m, 1H), 3.93 (s, 3H), 3.89 (s, 3H).

### Step 8: 6-bromo-7-methoxy-1H-indazole

To a solution of (E)-1-(4-bromo-2-fluoro-3-methoxy-phenyl)-N-methoxy-methanimine (17.5 g, 66.77 mmol, 1 equiv.) in DME (200 mL) was added N₂H4·H₂O (34.11 g, 667.75 mmol, 33.05 mL, 98% purity, 10 equiv.). The mixture was stirred at 90° C. for 12 hours. The reaction mixture was quenched by the addition of acetone (5 mL) at 25° C., filtered, washed with EtOAc (50 mL), concentrated under reduced pressure to give a residue, which was purified by flash silica gel chromatography (0~35% ethyl acetate/petroleum ether) to afford 6-bromo-7-methoxy-1H-indazole (4 g) as a white solid.

¹H NMR (400 MHz, DMSO-d₆) δ=13.52 (s, 1H), 8.14 (s, 1H), 7.46 (d, J=8.4 Hz, 1H), 7.24 (d, J=8.4 Hz, 1H), 3.97 (s, 3H).

### Step 9: 6-bromo-3-iodo-7-methoxy-1H-indazole

To a solution of 6-bromo-7-methoxy-1H-indazole (7.4 g, 32.59 mmol, 1 equiv.) in DMF (10 mL) was added potassium carbonate (9.01 g, 65.18 mmol, 2 equiv.) and I₂ (8.27 g, 32.59 mmol, 6.56 mL, 1 equiv.). The mixture solution was stirred at 25° C. for 12 hours. The reaction mixture was diluted with water (20 mL), filtered, and concentrated to afford 6-bromo-3-iodo-7-methoxy-1H-indazole (11.5 g) as a red oil.

LC-MS (ESI): m/z=352.6 [M+H]⁺

### Step 10: 6-bromo-3-iodo-7-methoxy-1-methyl-indazole

To a solution of 6-bromo-3-iodo-7-methoxy-1H-indazole (11.5 g, 28.67 mmol, 1 equiv.) in DMF (120 mL) was added Mel (8.14 g, 57.34 mmol, 3.57 mL, 2 equiv.) and potassium carbonate (9.91 g, 71.68 mmol, 2.5 equiv.). The mixture was stirred at 25° C. for 12 hours. The reaction mixture was concentrated under reduced pressure to remove solvent to give a residue, which was purified by flash silica gel chromatography (0~10% ethyl acetate/petroleum ether) to afford 6-bromo-3-iodo-7-methoxy-1-methyl-indazole (3 g) as a yellow oil

LC-MS (ESI): m/z=366.7 [M+H]⁺

### Step 11: 6-bromo-3-(2,6-dibenzyloxy-3-pyridyl)-7-methoxy-1-methyl-indazole

To a solution of 6-bromo-3-iodo-7-methoxy-1-methyl-indazole (5.3 g, 14.44 mmol, 1 equiv.) in THF (10 mL) and H₂O (1 mL) were added Pd(dppf)Cl₂ (1.06 g, 1.44 mmol, 0.1 equiv.), 2,6-dibenzyloxy-3-(4,4,5,5-tetramethyl-1,3,2-dioxaborolan-2-yl)pyridine (6.03 g, 14.44 mmol, 1 equiv.), and cesium carbonate (14.12 g, 43.33 mmol, 3 equiv.). The mixture was stirred at 60° C. for 16 hours. The reaction mixture was concentrated under reduced pressure to remove solvent to give a residue, which was purified by flash silica gel chromatography (0~9% ethyl acetate/petroleum ether) to afford 6-bromo-3-(2,6-dibenzyloxy-3-pyridyl)-7-methoxy-1-methyl-indazole (3 g) as a yellow oil.

¹H NMR (400 MHz, DMSO-d₆) δ=7.89 (d, J=8.0 Hz, 1H), 7.54-7.24 (m, 11H), 7.15 (d, J=8.8 Hz, 1H), 6.60 (d, J=8.0 Hz, 1H), 5.44 (d, J=6.8 Hz, 4H), 4.23 (s, 3H), 3.95 (s, 3H).

### Step 12: tert-butyl N-[3-(2,6-dibenzyloxy-3-pyridyl)-7-methoxy-1-methyl-indazol-6-yl]carbamate

A mixture of 6-bromo-3-(2,6-dibenzyloxy-3-pyridyl)-7-methoxy-1-methyl-indazole (5.1 g, 9.62 mmol, 1 equiv.), NH₂Boc (4.43 g, 28.85 mmol, 3 equiv. HCl), XantPhos (1.11 g, 1.92 mmol, 0.2 equiv.), cesium carbonate (9.40 g, 28.85 mmol, 3 equiv.), and Pd(OAc)₂ (215.87 mg, 961.52 mol, 0.1 equiv.) in dioxane (100 mL) was degassed and purged with N₂ three times. The mixture was stirred at 100° C. for 12 hours under a N₂ atmosphere. The reaction mixture was concentrated under reduced pressure to remove solvent to give a residue, which was purified by flash silica gel chromatography (0-15% ethyl acetate/petroleum ether) to afford tert-butyl N-[3-(2,6-dibenzyloxy-3-pyridyl)-7-methoxy-1-methyl-indazol-6-yl]carbamate (4 g) as a yellow oil LC-MS (ESI): m/z=567.4 [M+H]⁺

### Step 13: tert-butyl N-[3-(2,6-dioxo-3-piperidyl)-7-methoxy-1-methyl-indazol-6-yl]carbamate

To a solution of tert-butyl N-[3-(2,6-dibenzyloxy-3-pyridyl)-7-methoxy-1-methyl-indazol-6-yl]carbamate (5.4 g, 9.53 mmol, 1 equiv.) in THF (1 mL) and EtOAc (1 mL) were added Pd on carbon (10 wt %, 2.03 g, 1.91 mmol, 0.2 equiv.) and Pd(OH)₂ on carbon (20 wt %, 1.34 g, 1.91 mmol, 0.2 equiv.) under a N₂ atmosphere. The suspension was degassed and purged with H₂ three times. The mixture was stirred under H₂ (15 psi) at 25° C. for 16 hours. The mixture was filtered and concentrated to afford tert-butyl N-[3-(2,6-dioxo-3-piperidyl)-7-methoxy-1-methyl-indazol-6-yl]carbamate (7 g) as a pink oil.

¹H NMR (400 MHz, DMSO-d₆) δ=10.90 (s, 1H), 8.64 (s, 1H), 7.36 (d, J=5.6 Hz, 1H), 7.23 (s, 1H), 4.32 (d, J=5.6 Hz, 1H), 4.12 (s, 3H), 3.82 (s, 3H), 2.73-2.59 (m, 2H), 1.99 (s, 2H), 1.47 (s, 9H).

### Step 14: 3-(6-amino-7-methoxy-1-methyl-indazol-3-yl)piperidine-2,6-dione

To a solution of tert-butyl N-[3-(2,6-dioxo-3-piperidyl)-7-methoxy-1-methyl-indazol-6-yl]carbamate (3.7 g, 9.53 mmol, 1 equiv.) in HCl/dioxane (25 mL). The mixture was stirred at 25° C. for 2 hours. The reaction mixture was concentrated under reduced pressure to remove solvent to afford 3-(6-amino-7-methoxy-1-methyl-indazol-3-yl)piperidine-2,6-dione (56 g) as a white solid.

LC-MS (ESI): m/z=289.0 [M+H]⁺

¹H NMR (400 MHz, CD₃OD-d₄) δ=7.68 (d, J=8.4 Hz, 1H), 7.13 (d, J=8.4 Hz, 1H), 4.43 (dd, J=5.2, 9.6 Hz, 1H), 4.33-4.23 (m, 3H), 4.05 (s, 3H), 2.93-2.71 (m, 2H), 2.59-2.44 (m, 1H), 2.41-2.27 (m, 1H).

### Step 15: tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate

To a solution of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (110 mg, 141.76 mol, 1 equiv.) in pyridine (1 mL) was added EDCI (40.76 mg, 212.64 mol, 1.5 equiv.) and 3-(6-amino-7-methoxy-1-methyl-indazol-3-yl)piperidine-2,6-dione (49.04 mg, 170.11 mol, 1.2 equiv.). The mixture was stirred at 25° C. for 2 hours. The reaction mixture was diluted with water (10 mL) and extracted with ethyl acetate (30 mL). The combined organic layers were dried over sodium sulfate, filtered, and concentrated under reduced pressure to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate (140 mg) as a yellow solid.

LC-MS (ESI): m/z=524.2 [M12+H]⁺.

### Step 16: 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate (140 mg, 133.81 mol, 1 equiv.) in TFA (1 mL) and DCM (1 mL). The mixture was stirred at 25° C. for 16 hours. The reaction mixture was filtered, and the filtrate was concentrated under reduced pressure to give a residue, which was purified by reversed-phase HPLC to afford 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid (59.13 mg) as an off-white solid.

LC-MS (ESI): m/z=990.7 [M+H]⁺

¹H NMR (400 MHz, DMSO-d₆) δ=13.15-12.72 (m, 1H), 12.69-12.30 (m, 1H), 10.89 (s, 1H), 9.84 (s, 1H), 8.13 (s, 1H), 8.06-7.93 (m, 2H), 7.79 (d, J=8.0 Hz, 1H), 7.62 (d, J=7.2 Hz, 1H), 7.49-7.42 (m, 4H), 7.36 (td, J=7.6, 10.8 Hz, 2H), 7.13-7.06 (m, 1H), 6.97 (d, J=8.8 Hz, 1H), 6.89 (d, J=8.0 Hz, 1H), 6.63 (d, J=7.8 Hz, 1H), 4.98 (s, 2H), 4.33 (dd, J=5.2, 10.0 Hz, 1H), 4.13 (s, 3H), 3.98 (t, J=5.6 Hz, 2H), 3.92 (t, J=5.6 Hz, 2H), 3.88 (s, 3H), 3.15 (s, 2H), 3.03 (t, J=5.6 Hz, 2H), 2.98-2.88 (m, 2H), 2.69-2.57 (m, 2H), 2.24 (s, 2H), 2.21-2.12 (m, 2H), 1.91 (s, 3H), 1.78 (d, J=9.2 Hz, 4H), 1.48-1.40 (m, 2H), 1.37-1.25 (m, 3H)

### Example 2: 6-(8-(benzo[d]thiazol-2-ylcarbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid (Compound 106)

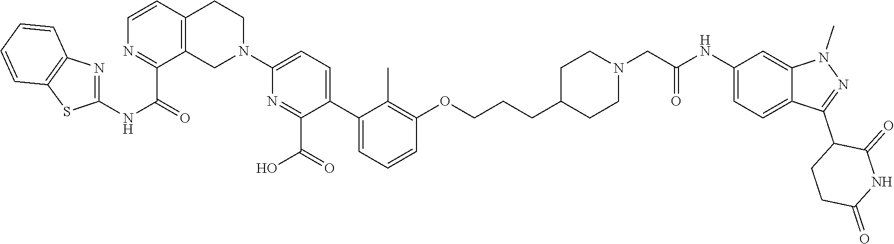 [SMILES: Cc1c(OCCCC2CCN(CC(=O)Nc3ccc4c(C5CCC(=O)NC5=O)nn(C)c4c3)CC2)cccc1-c1ccc(N2CCc3ccnc(C(=O)Nc4nc5ccccc5s4)c3C2)nc1C(=O)O]

### Step 1: tert-butyl 4-[3-(3-bromo-2-methyl-phenoxy)propyl]piperidine-1-carboxylate

A mixture of 3-bromo-2-methyl-phenol (1.95 g, 10.43 mmol, 1 equiv.), tert-butyl 4-(3-hydroxypropyl)piperidine-1-carboxylate (2.79 g, 11.47 mmol, 1.1 equiv.), and 2-(tributyl-phosphanylidene)acetonitrile (3.77 g, 15.64 mmol, 1.5 equiv.) in toluene (40 mL) was degassed and purged with N₂ three times. The mixture was stirred at 120° C. for 16 hours under a N₂ atmosphere. The reaction mixture was concentrated under reduced pressure to give a residue, which was purified by flash silica gel chromatography (6% ethyl acetate/petroleum ether) to afford tert-butyl 4-[3-(3-bromo-2-methyl-phenoxy)propyl]piperidine-1-carboxylate (2 g) as a yellow oil.

LC-MS (ESI): m/z=356.2 [M−56+H]⁺.

¹H NMR (400 MHz, DMSO-d₆) δ=7.17-7.12 (m, 1H), 7.12-7.05 (m, 1H), 6.96 (d, J=8.0 Hz, 1H), 3.97 (t, J=6.4 Hz, 2H), 3.90 (s, 2H), 3.30 (s, 1H), 2.67 (s, 2H), 2.23 (s, 3H), 1.79-1.71 (m, 2H), 1.66 (d, J=12.4 Hz, 2H), 1.38 (s, 9H), 1.37-1.32 (m, 2H), 1.02-0.91 (m, 2H).

### Step 2: 4-[3-(3-bromo-2-methyl-phenoxy)propyl]piperidine

A mixture of tert-butyl 4-[3-(3-bromo-2-methyl-phenoxy)propyl]piperidine-1-carboxylate (2 g, 4.85 mmol, 1 equiv.) in DCM (25 mL) and TFA (5 mL) was degassed and purged with N₂ three times. The mixture was stirred at 25° C. for 16 hours under a N₂ atmosphere. The reaction mixture was concentrated under reduced pressure to afford 4-[3-(3-bromo-2-methyl-phenoxy)propyl]piperidine (1.7 g, crude) as a white solid.

### Step 3: ethyl 2-[4-[3-(3-bromo-2-methyl-phenoxy)propyl]-1-piperidyl]acetate

A mixture of 4-[3-(3-bromo-2-methyl-phenoxy)propyl]piperidine (1 g, 3.20 mmol, 1 equiv.), ethyl 2-bromoacetate (534.84 mg, 3.20 mmol, 354.43 L, 1 equiv.), and potassium carbonate (2.21 g, 16.01 mmol, 5 equiv.) in MeCN (10 mL) was degassed and purged with N₂ three times. The mixture was stirred at 60° C. for 2 hours under a N₂ atmosphere. The reaction mixture was filtered and concentrated under reduced pressure to afford ethyl 2-[4-[3-(3-bromo-2-methyl-phenoxy)propyl]-1-piperidyl]acetate (1.2 g, crude) as a yellow solid.

LC-MS (ESI): m/z=398.2 [M+H]⁺

¹H NMR (400 MHz, CDCl₃) δ=7.14 (d, J=7.6 Hz, 1H), 7.03-6.96 (m, 1H), 6.76 (d, J=8.4 Hz, 1H), 4.20 (q, J=7.2 Hz, 2H), 3.94 (t, J=6.4 Hz, 2H), 3.23 (s, 2H), 2.98 (d, J=10.4 Hz, 2H), 2.31 (s, 3H), 2.28-2.08 (m, 2H), 1.87-1.78 (m, 2H), 1.73 (d, J=12.0 Hz, 2H), 1.47-1.40 (m, 3H), 1.38-1.32 (m, 2H), 1.29 (t, J=7.2 Hz, 3H).

### Step 4: 2-[4-[3-(3-bromo-2-methyl-phenoxy)propyl]-1-piperidyl]acetic acid

A mixture of ethyl 2-[4-[3-(3-bromo-2-methyl-phenoxy)propyl]-1-piperidyl]acetate (1.2 g, 3.01 mmol, 1 equiv.) and LiOH·H₂O (379.25 mg, 9.04 mmol, 3 equiv.) in THF (10 mL) and H₂O (2.5 mL) was degassed and purged with N₂ three times. The mixture was stirred at 40° C. for 2 hours under a N₂ atmosphere. The reaction mixture was concentrated under reduced pressure to remove THF. The residue was diluted with H₂O (4 mL) and adjusted pH to 5-6. The residue was filtered and concentrated under reduced pressure to afford 2-[4-[3-(3-bromo-2-methyl-phenoxy)propyl]-1-piperidyl]acetic acid (1.1 g) as a yellow solid.

### Step 5: 3-(2,6-dibenzyloxy-3-pyridyl)-1-methyl-6-nitro-indazole

To a solution of (2,6-dibenzyloxy-3-pyridyl)boronic acid (4.5 g, 13.43 mmol, 1 equiv.) and 3-iodo-1-methyl-6-nitro-indazole (4.48 g, 14.77 mmol, 1.1 equiv.) in dioxane (45 mL) and H₂O (4.5 mL) was added sodium carbonate (4.27 g, 40.28 mmol, 3 equiv.) and Pd(dppf)Cl₂ (982.42 mg, 1.34 mmol, 0.1 equiv.). The mixture was stirred at 100° C. for 2 hours. The reaction mixture was filtered and concentrated under reduced pressure to give a residue, which was purified by column chromatography (SiO₂, petroleum ether/ethyl acetate=I/O to 5/1) to give 3-(2,6-dibenzyloxy-3-pyridyl)-1-methyl-6-nitro-indazole (8 g) as a yellow solid.

LC-MS (ESI): m/z=467.1 [M+H]⁺.

### Step 6: 3-(2,6-dibenzyloxy-3-pyridyl)-1-methyl-indazol-6-amine

A mixture of 3-(2,6-dibenzyloxy-3-pyridyl)-1-methyl-6-nitro-indazole (3.8 g, 8.15 mmol, 1 equiv.), Pd on carbon (10 wt %, 900 mg), and Pd(OH)₂ on carbon (20 wt %, 900 mg) in EtOH (40 mL) and THF (40 mL) was degassed and purged with H₂ and stirred at 50° C. for 12 hours under H₂ atmosphere (50 psi). The reaction mixture was filtered and concentrated under reduced pressure to give a residue, which was purified by column chromatography (SiO₂, petroleum ether/ethyl acetate=10/1 to 1/1) to give 3-(2,6-dibenzyloxy-3-pyridyl)-1-methyl-indazol-6-amine (7 g) as a pink solid.

LC-MS (ESI): m/z=437.1 [M+H]⁺.

¹H NMR (400 MHz, DMSO-d₆) δ=7.86 (d, J=8.4 Hz, 1H), 7.48-7.45 (m, 2H), 7.42-7.37 (m, 4H), 7.36-7.34 (m, 1H), 7.34-7.29 (m, 4H), 6.55 (d, J=8.0 Hz, 1H), 6.42-6.37 (m, 2H), 5.42 (d, J=10.4 Hz, 4H), 5.32 (s, 2H), 3.84 (s, 3H).

### Step 7: 3-(6-amino-1-methyl-indazol-3-yl)piperidine-2,6-dione

To a solution of 3-(2,6-dibenzyloxy-3-pyridyl)-1-methyl-indazol-6-amine (1.7 g, 3.89 mmol, 1 equiv.) in THF (15 mL) and EtOH (15 mL) was added Pd on carbon (10 wt %, 500 mg), Pd(OH)₂ on carbon (20 wt %, 500 mg), and acetic acid (233.88 mg, 3.89 mmol, 222.74 μL, 1 equiv.). The mixture was stirred at 50° C. for 24 hours. The reaction mixture was filtered and concentrated under reduced pressure to give a residue, which was purified by column chromatography (SiO₂, DCM:MeOH=100/1 to 10/1) to give 3-(6-amino-1-methyl-indazol-3-yl)piperidine-2,6-dione (600 mg) as a pink solid.

LC-MS (ESI): m/z=259.1 [M+H]⁺

¹H NMR (400 MHz, CDCl₃) δ=7.93 (s, 1H), 7.45 (d, J=8.8 Hz, 1H), 6.58 (d, J=8.8 Hz, 1H), 6.50 (s, 1H), 4.24 (t, J=6.0 Hz, 1H), 3.89 (s, 3H), 3.03-2.95 (m, 1H), 2.70-2.62 (m, 1H), 2.54-2.45 (m, 1H), 2.40-2.31 (m, 1H).

### Step 8: 2-[4-[3-(3-bromo-2-methyl-phenoxy)propyl]-1-piperidyl]-N-[3-(2,6-dioxo-3-piperidyl)-1-methyl-indazol-6-yl]acetamide

A mixture of 2-[4-[3-(3-bromo-2-methyl-phenoxy)propyl]-1-piperidyl]acetic acid (170 mg, 459.11 mol, 1 equiv.), 3-(6-amino-1-methyl-indazol-3-yl)piperidine-2,6-dione (142.29 mg, 550.93 mol, 1.2 equiv.), and EDCI (132.02 mg, 688.67 mol, 1.5 equiv.) in pyridine (4 mL) was degassed and purged with N₂ three times. The mixture was stirred at 25° C. for 16 hours under a N₂ atmosphere. The reaction mixture was diluted with H₂O (10 mL) and extracted with DCM (45 mL). The combined organic layers were dried over sodium sulfate, filtered, and concentrated under reduced pressure to give a residue, which was purified by flash silica gel chromatography (100% ethyl acetate/petroleum ether) to afford 2-[4-[3-(3-bromo-2-methyl-phenoxy)propyl]-1-piperidyl]-N-[3-(2,6-dioxo-3-piperidyl)-1-methyl-indazol-6-yl]acetamide (228 mg) as a white solid.

LC-MS (ESI): m/z=610.4 [M+H]⁺

¹H NMR (400 MHz, DMSO-d₆) δ=10.88 (s, 1H), 9.82 (s, 1H), 8.05 (s, 1H), 7.63 (d, J=8.4 Hz, 1H), 7.24-7.19 (m, 1H), 7.17-7.13 (m, 1H), 7.09 (t, J=8.0 Hz, 1H), 6.97 (d, J=8.0 Hz, 1H), 4.35-4.30 (m, 1H), 3.98 (t, J=6.0 Hz, 2H), 3.92 (s, 3H), 3.13 (s, 2H), 2.88 (d, J=10.8 Hz, 2H), 2.65-2.59 (m, 2H), 2.24 (s, 3H), 2.18-2.13 (m, 3H), 1.79-1.74 (m, 2H), 1.71-1.67 (m, 2H), 1.42-1.36 (m, 2H), 1.29 (s, 4H).

### Step 9: N-(3-(2,6-dioxopiperidin-3-yl)-1-methyl-1H-indazol-6-yl)-2-(4-(3-(2-methyl-3-(4,4,5,5-tetramethyl-1,3,2-dioxaborolan-2-yl)phenoxy)propyl)piperidin-1-yl)acetamide

2-[4-[3-(3-bromo-2-methyl-phenoxy)propyl]-1-piperidyl]-N-[3-(2,6-dioxo-3-piperidyl)-1-methyl-indazol-6-yl]acetamide (250 mg, 409.47 mol, 1 equiv.), 4,4,5,5-tetramethyl-1,3,2-dioxaborolane (262.02 mg, 2.05 mmol, 297.07 L, 5 equiv.), TEA (124.30 mg, 1.23 mmol, 170.98 L, 3 equiv.), and Pd(dppf)Cl₂ (29.96 mg, 40.95 mol, 0.1 equiv.) were taken up into a microwave tube in THF (1 mL) and MeCN (3 mL). The sealed tube was heated at 100° C. for 1 hour under microwave. The reaction mixture was diluted with H₂O (5 mL) and extracted with DCM (15 mL). The combined organic layers were dried over sodium sulfate, filtered, and concentrated under reduced pressure to give a residue, which was purified by prep-HPLC to afford N-(3-(2,6-dioxopiperidin-3-yl)-1-methyl-1H-indazol-6-yl)-2-(4-(3-(2-methyl-3-(4,4,5,5-tetramethyl-1,3,2-dioxaborolan-2-yl)phenoxy)propyl)piperidin-1-yl)acetamide (165 mg) as a red solid.

LC-MS (ESI): m/z=658.6 [M+H]⁺

¹H NMR (400 MHz, DMSO-d₆) δ=10.88 (s, 1H), 9.83 (s, 1H), 8.05 (s, 1H), 7.63 (d, J=8.4 Hz, 1H), 7.22 (d, J=9.2 Hz, 1H), 7.19-7.16 (m, 1H), 7.15-7.09 (m, 1H), 7.01 (d, J=8.8 Hz, 1H), 4.32 (d, J=4.4, 9.6 Hz, 1H), 3.94 (s, 2H), 3.92 (s, 3H), 3.13 (s, 2H), 2.88 (d, J=10.0 Hz, 2H), 2.68-2.60 (m, 2H), 2.32 (s, 3H), 2.26-2.08 (m, 4H), 1.77 (d, J=6.8 Hz, 2H), 1.69 (d, J=7.2 Hz, 2H), 1.44-1.36 (m, 2H), 1.29 (s, 15H).

### Step 10: 5,6,7,8-tetrahydro-2,7-naphthyridin-1-ol

To a solution of 2,7-naphthyridin-1-ol (10 g, 68.42 mmol, 1 equiv.) in MeOH (100 mL) was added PtO₂ (7.77 g, 34.21 mmol, 0.5 equiv.) under a N₂ atmosphere. The suspension was degassed and purged with H₂ three times. The mixture was stirred under H₂ (30 psi.) at 30° C. for 16 hours. The reaction mixture was filtered, and the filter cake was immersed with MeOH (50 mL). The filtrate was concentrated under reduced pressure to give a residue, which was purified by flash silica gel chromatography (0~25% MeOH/DCM) to afford 5,6,7,8-tetrahydro-2,7-naphthyridin-1-ol (6 g) as a yellow solid.

LC-MS (ESI): m/z=151.3 [M+H]⁺

¹H NMR (400 MHz, DMSO-d₆) δ=7.11 (d, J=6.4 Hz, 1H), 5.92 (d, J=6.8 Hz, 1H), 3.45 (s, 2H), 3.17 (s, 1H), 2.83 (t, J=5.6 Hz, 2H), 2.43 (t, J=5.6 Hz, 2H).

### Step 11: 7-benzyl-6,8-dihydro-5H-2,7-naphthyridin-1-ol

A mixture of 5,6,7,8-tetrahydro-2,7-naphthyridin-1-ol (6 g, 39.95 mmol, 1 equiv.) and benzaldehyde (6.36 g, 59.93 mmol, 6.06 mL, 1.5 equiv.) in DCM (60 mL) was stirred at 0° C. for 0.5 hours. To this mixture, NaBH(OAc)₃ (25.40 g, 119.86 mmol, 3 equiv.) was added. The mixture was stirred at 0-25° C. for 16 hours under a N₂ atmosphere. The reaction mixture was concentrated under reduced pressure to give a residue, which was purified by flash silica gel chromatography (0-12% MeOH/DCM) to afford 7-benzyl-6,8-dihydro-5H-2,7-naphthyridin-1-ol (7 g) as a yellow solid.

LC-MS (ESI): m/z=241.2 [M+H]⁺

¹H NMR (400 MHz, DMSO-d₆) δ=11.66-11.08 (m, 1H), 7.34 (t, J=3.6 Hz, 4H), 7.27 (d, J=3.6, 8.0 Hz, 1H), 7.17-7.10 (m, 1H), 5.97 (d, J=2.8, 6.4 Hz, 1H), 3.64 (s, 2H), 3.15 (s, 2H), 2.61 (s, 3H), 1.93-1.87 (m, 1H).

### Step 12: 2-benzyl-8-chloro-3,4-dihydro-1H-2,7-naphthyridine

A mixture of 7-benzyl-6,8-dihydro-5H-2,7-naphthyridin-1-ol (5.7 g, 23.72 mmol, 1 equiv.) in MeCN (30 mL) and POCl₃ (30 mL) was degassed and purged with N₂ three times. The mixture was stirred at 90° C. for 16 hours under a N₂ atmosphere. The reaction mixture was quenched by the addition of warm water (30 mL) at 25° C., and concentrated under reduced pressure to give a residue, which was purified by flash silica gel chromatography (0~8% MeOH/DCM) to afford 2-benzyl-8-chloro-3,4-dihydro-1H-2,7-naphthyridine (9 g, crude) as a brown oil.

LC-MS (ESI): m/z=259.2 [M+H]⁺

¹H NMR (400 MHz, DMSO-d₆) δ=8.29 (d, J=4.8 Hz, 1H), 7.75-7.68 (m, 2H), 7.52-7.46 (m, 3H), 7.37 (s, 1H), 4.61-4.45 (m, 2H), 4.27 (s, 2H), 3.58-3.50 (m, 2H), 3.16 (s, 2H).

### Step 13: methyl 7-benzyl-6,8-dihydro-5H-2,7-naphthyridine-1-carboxylate

To a solution of 2-benzyl-8-chloro-3,4-dihydro-1H-2,7-naphthyridine (9 g, 34.78 mmol, 1 equiv.) in MeOH (100 mL) was added Pd(dppf)Cl₂ (2.55 g, 3.48 mmol, 0.1 equiv.) and TEA (10.56 g, 104.35 mmol, 14.52 mL, 3 equiv.) under a N₂ atmosphere. The suspension was degassed and purged with CO three times. The mixture was stirred under CO (50 psi.) at 80° C. for 16 hours. The reaction mixture was filtered and concentrated under reduced pressure to give a residue, which was purified by flash silica gel chromatography (0~55% ethyl acetate/petroleum ether) to afford methyl 7-benzyl-6,8-dihydro-5H-2,7-naphthyridine-1-carboxylate (4.3 g, crude) as a brown oil.

LC-MS (ESI): m/z=283.3 [M+H]⁺

¹H NMR (400 MHz, DMSO) δ=8.37 (d, J=4.8 Hz, 1H), 7.34 (d, J=4.4 Hz, 5H), 7.27 (d, J=4.0, 8.4 Hz, 1H), 3.82-3.76 (m, 5H), 3.69 (s, 2H), 2.89 (t, J=5.6 Hz, 2H), 2.66 (t, J=5.6 Hz, 2H).

### Step 14: methyl 5,6,7,8-tetrahydro-2,7-naphthyridine-1-carboxylate

To a solution of methyl 7-benzyl-6,8-dihydro-5H-2,7-naphthyridine-1-carboxylate (2.3 g, 8.15 mmol, 1 equiv.) in MeOH (50 mL) was added Pd(OH)₂ on carbon (10 wt %, 1.14 g, 814.63 mol, 0.1 equiv.) under a N₂ atmosphere. The suspension was degassed and purged with H₂ three times. The mixture was stirred under H₂ (15 psi.) at 25° C. for 16 hours. The reaction mixture was filtered, and the filter cake was immersed with MeOH (100 mL). The filtrate was concentrated under reduced pressure to give a residue, which was purified by flash silica gel chromatography (0~12% MeOH/DCM) to afford methyl 5,6,7,8-tetrahydro-2,7-naphthyridine-1-carboxylate (1.16 g) as a yellow solid.

LC-MS (ESI): m/z=193.2 [M+H]⁺

¹H NMR (400 MHz, DMSO) δ=8.33 (d, J=4.8 Hz, 1H), 7.30 (d, J=4.4 Hz, 1H), 4.00 (s, 2H), 3.83 (s, 3H), 2.95-2.90 (m, 2H), 2.76 (t, J=5.6 Hz, 2H).

### Step 15: methyl 7-(5-bromo-6-tert-butoxycarbonyl-2-pyridyl)-6,8-dihydro-5H-2,7-naphthyridine-1-carboxylate

A mixture of methyl 5,6,7,8-tetrahydro-2,7-naphthyridine-1-carboxylate (1.16 g, 6.03 mmol, 1 equiv.), tert-butyl 3-bromo-6-chloro-pyridine-2-carboxylate (1.77 g, 6.03 mmol, 1 equiv.), and CsF (2.75 g, 18.10 mmol, 668.33 L, 3 equiv.) in DMA (20 mL) was degassed and purged with N₂ three times. The mixture was stirred at 120° C. for 16 hours under a N₂ atmosphere. The reaction mixture was diluted with H₂O (15 mL) and extracted with ethyl acetate (30 mL). The combined organic layers were washed with brine (30 mL), dried over sodium sulfate, filtered, and concentrated under reduced pressure to give a residue, which was purified by flash silica gel chromatography (0-58% ethyl acetate/petroleum ether) to afford methyl 7-(5-bromo-6-tert-butoxycarbonyl-2-pyridyl)-6,8-dihydro-5H-2,7-naphthyridine-1-carboxylate (2 g) as a yellow oil.

LC-MS (ESI): m/z=448.1 [M+H]⁺

¹H NMR (400 MHz, DMSO) δ=8.43 (d, J=4.4 Hz, 1H), 7.81 (d, J=8.8 Hz, 1H), 7.44 (d, J=4.4 Hz, 1H), 6.96 (d, J=8.8 Hz, 1H), 4.96 (s, 2H), 3.89 (s, 3H), 2.94 (s, 2H), 2.78 (s, 2H), 1.54 (s, 9H).

### Step 16: 7-(5-bromo-6-tert-butoxycarbonyl-2-pyridyl)-6,8-dihydro-5H-2,7-naphthyridine-1-carboxylic acid

A mixture of methyl 7-(5-bromo-6-tert-butoxycarbonyl-2-pyridyl)-6,8-dihydro-5H-2,7-naphthyridine-1-carboxylate (1.2 g, 2.68 mmol, 1 equiv.) and LiOH·H₂O (336.97 mg, 8.03 mmol, 3 equiv.) in H₂O (3 mL) and THF (9 mL) was stirred at 40° C. for 2 hours under a N₂ atmosphere. The reaction mixture was concentrated under reduced pressure to remove solvent. The residue was adjusted to pH=5-6 by HCl (1 M) and extracted with DCM (15 mL). The combined organic layers were dried over sodium sulfate, filtered, and concentrated under reduced pressure to afford 7-(5-bromo-6-tert-butoxycarbonyl-2-pyridyl)-6,8-dihydro-5H-2,7-naphthyridine-1-carboxylic acid (1.1 g) as a yellow solid.

### Step 17: tert-butyl 6-[8-(1,3-benzothiazol-2-ylcarbamoyl)-3,4-dihydro-1H-2,7-naphthyridin-2-yl]-3-bromo-pyridine-2-carboxylate

A mixture of 7-(5-bromo-6-tert-butoxycarbonyl-2-pyridyl)-6,8-dihydro-5H-2,7-naphthyridine-1-carboxylic acid (1.1 g, 2.53 mmol, 1 equiv.), 1,3-benzothiazol-2-amine (380.44 mg, 2.53 mmol, 1 equiv.), TCFH (852.82 mg, 3.04 mmol, 1.2 equiv.), and NMI (727.86 mg, 8.87 mmol, 706.66 L, 3.5 equiv.) in MeCN (20 mL) was stirred at 25° C. for 16 hours under a N₂ atmosphere. The reaction mixture was concentrated under reduced pressure to give a residue, which was purified by flash silica gel chromatography (0~49% ethyl acetate/petroleum ether) to afford tert-butyl 6-[8-(1,3-benzothiazol-2-ylcarbamoyl)-3,4-dihydro-1H-2,7-naphthyridin-2-yl]-3-bromo-pyridine-2-carboxylate (1 g) as a yellow solid.

LC-MS (ESI): m/z=566.3 [M+H]⁺

¹H NMR (400 MHz, DMSO) δ=12.35 (s, 1H), 8.55 (d, J=4.8 Hz, 1H), 8.05 (d, J=8.0 Hz, 1H), 7.86-7.79 (m, 2H), 7.65-7.62 (m, 1H), 7.55 (d, J=5.2 Hz, 1H), 7.51-7.46 (m, 1H), 7.00-6.97 (m, 1H), 5.18 (s, 2H), 3.88 (t, J=5.6 Hz, 2H), 3.04 (t, J=6.0 Hz, 2H), 1.47 (s, 9H).

### Step 18: tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate

A mixture of tert-butyl 6-[8-(1,3-benzothiazol-2-ylcarbamoyl)-3,4-dihydro-1H-2,7-naphthyridin-2-yl]-3-bromo-pyridine-2-carboxylate (200 mg, 353.06 mol, 1 equiv.) and TEA (107.18 mg, 1.06 mmol, 147.43 μL, 3 equiv.) in THF (4 mL) was degassed and purged with N₂ three times at 0° C. followed by the addition of SEM-Cl (88.29 mg, 529.60 mol, 93.73 L, 1.5 equiv.). The mixture was stirred at 0-25° C. for 2 hours under a N₂ atmosphere. The reaction mixture was concentrated under reduced pressure to give a residue, which was purified by flash silica gel chromatography (24% ethyl acetate/petroleum ether) to afford tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (150 mg) as a white solid.

LC-MS (ESI): m/z=696.3 [M+H]⁺

¹H NMR (400 MHz, DMSO-d₆) δ=8.46 (d, J=4.8 Hz, 1H), 8.08 (d, J=8.0 Hz, 1H), 7.84 (d, J=8.0 Hz, 1H), 7.77 (d, J=9.2 Hz, 1H), 7.50 (d, J=7.6 Hz, 1H), 7.49-7.46 (m, 1H), 7.45-7.39 (m, 1H), 6.92 (d, J=9.2 Hz, 1H), 5.62 (s, 2H), 4.73 (s, 2H), 3.79 (t, J=5.2 Hz, 2H), 3.39-3.34 (m, 2H), 3.04 (s, 2H), 1.32 (s, 9H), 0.49 (t, J=8.0 Hz, 2H), −0.25 (s, 9H).

### Step 19: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate

A mixture of N-(3-(2,6-dioxopiperidin-3-yl)-1-methyl-1H-indazol-6-yl)-2-(4-(3-(2-methyl-3-(4,4,5,5-tetramethyl-1,3,2-dioxaborolan-2-yl)phenoxy)propyl)piperidin-1-yl)acetamide (50 mg, 76.03 mol, 0.6 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (52.97 mg, 76.03 mol, 0.6 equiv.), cesium carbonate (3 M, 126.72 L, 3 equiv.), and XPhos Pd G3 (11.14 mg, 12.67 mol, 0.1 equiv.) in dioxane (2 mL) was degassed and purged with N₂ three times. The mixture was stirred at 90° C. for 16 hours under a N₂ atmosphere. The reaction mixture was concentrated under reduced pressure to give a residue, which was purified by prep-TLC (SiO₂, DCM:MeOH=15:1) to afford tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate (50 mg, crude) as a yellow oil.

LC-MS (ESI): m/z=547.6 [M12+H]⁺.

### Step 20: 6-(8-(benzo[d]thiazol-2-ylcarbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid

A mixture of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate (45 mg, 39.22 mol, 1 equiv.) in TFA (0.5 mL) and DCM (1 mL) was degassed and purged with N₂ three times. The mixture was stirred at 25° C. for 16 hours under a N₂ atmosphere. The reaction mixture was concentrated under reduced pressure to give a residue, which was purified by prep-HPLC to afford 6-(8-(benzo[d]thiazol-2-ylcarbamoyl)-3,4-dihydro-2,7-naphthyridin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid (8.24 mg) as a yellow solid.

LC-MS (ESI): m/z=961.4 [M+H]⁺

¹H NMR (400 MHz, DMSO-d₆) δ=10.87 (s, 1H), 9.82 (s, 1H), 8.56 (d, J=4.8 Hz, 1H), 8.22 (s, 1H), 8.07-8.02 (m, 2H), 7.81 (d, J=7.6 Hz, 1H), 7.62 (d, J=8.4 Hz, 1H), 7.58 (d, J=5.2 Hz, 1H), 7.51-7.42 (m, 2H), 7.36 (s, 1H), 7.22 (d, J=1.6, 8.8 Hz, 1H), 7.08 (d, J=8.4 Hz, 1H), 7.00 (d, J=2.4, 4.8 Hz, 1H), 6.87 (d, J=8.0 Hz, 1H), 6.67 (d, J=7.6 Hz, 1H), 5.23 (s, 2H), 4.32 (d, J=5.2, 9.6 Hz, 1H), 3.97 (t, J=5.6 Hz, 4H), 3.92 (s, 3H), 3.12 (s, 2H), 3.09-3.06 (m, 2H), 2.88 (d, J=8.8 Hz, 2H), 2.64-2.60 (m, 1H), 2.20-2.13 (m, 4H), 1.92 (s, 3H), 1.83-1.81 (m, 1H), 1.80-1.75 (m, 2H), 1.71-1.67 (m, 2H), 1.45-1.40 (m, 2H), 1.30 (s, 3H), 1.26-1.22 (m, 1H).

### Example 3: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid (Compound 107)

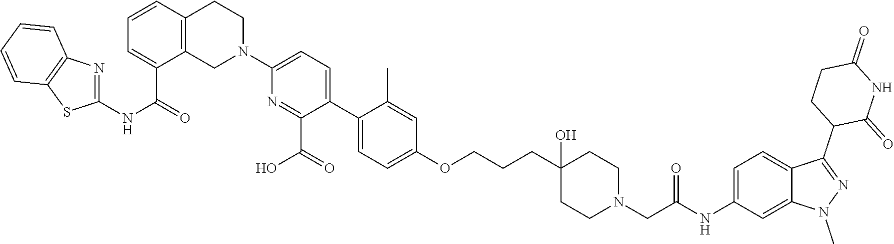 [SMILES: Cc1cc(OCCCC2(O)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: tert-butyl 4-allyl-4-((2-(trimethylsilyl)ethoxy)methoxy)piperidine-1-carboxylate

A mixture of tert-butyl 4-allyl-4-hydroxypiperidine-1-carboxylate (3.0 g, 12.43 mmol, 1.0 equiv.), TEA (6.29 g, 62.16 mmol, 8.65 mL, 5.0 equiv.), DMAP (303.74 mg, 2.49 mmol, 0.2 equiv.) in THF (20 mL) was degassed and purged with N₂ three times followed by the addition of (2-(chloromethoxy)ethyl)trimethylsilane (8.29 g, 49.73 mmol, 8.80 mL, 4.0 equiv.) at 0° C. The mixture was stirred at 40° C. for 16 hours under a N₂ atmosphere. The reaction mixture was filtered, and the filter liquid was concentrated under reduced pressure to give a residue, which was purified by flash silica gel chromatography (0-6% ethyl acetate/petroleum ether) to give tert-butyl 4-allyl-4-((2-(trimethylsilyl)ethoxy)methoxy)piperidine-1-carboxylate (2 g) as a colorless oil.

¹H NMR (400 MHz, DMSO-d₆) δ=5.83-5.71 (m, 1H), 5.07 (d, J=4.8 Hz, 1H), 5.04 (s, 1H), 4.71 (s, 2H), 3.64-3.55 (m, 4H), 3.05 (s, 2H), 2.29 (d, J=7.2 Hz, 2H), 1.66 (d, J=13.2 Hz, 2H), 1.38 (s, 11H), 0.89-0.81 (m, 2H), 0.00 (s, 9H)

### Step 2: tert-butyl 4-(3-hydroxypropyl)-4-((2-(trimethylsilyl)ethoxy)methoxy)piperidine-1-carboxylate

A mixture of tert-butyl 4-allyl-4-((2-(trimethylsilyl)ethoxy)methoxy)piperidine-1-carboxylate (1.2 g, 3.23 mmol, 1.0 equiv.) in THF (30 mL) was degassed and purged with N₂ three times followed by the addition of BH₃·THF (1 M, 3.23 mL, 1.0 equiv.) at 0° C. The mixture was stirred at 25° C. for 12 hours. NaOH (129.18 mg, 3.23 mmol, 1.0 equiv.) was added at 0° C. followed by H₂O₂ (648.01 mg, 5.72 mmol, 549.16 L, 30% purity, 1.77 equiv.). The mixture was stirred at 25° C. for 5 hours under a N₂ atmosphere. The reaction mixture was quenched by the addition of sodium sulfite (saturated solution, 100 mL) at 25° C. and extracted with ethyl acetate (300 mL). The combined organic layers were washed with brine (150 mL), dried over sodium sulfate, then concentrated (below 30° C.) under reduced pressure to give tert-butyl 4-(3-hydroxypropyl)-4-((2-(trimethylsilyl)ethoxy)methoxy)piperidine-1-carboxylate (2 g) as a colorless oil.

¹H NMR (400 MHz, DMSO-d₆) δ=4.65 (s, 2H), 3.63-3.54 (m, 4H), 3.38-3.33 (m, 1H), 3.05 (s, 2H), 1.67 (d, J=13.2 Hz, 2H), 1.51-1.42 (m, 2H), 1.38 (s, 9H), 1.36-1.21 (m, 4H), 0.87-0.83 (m, 2H), 0.00 (s, 9H)

### Step 3: tert-butyl 4-(3-(4-bromo-3-methylphenoxy)propyl)-4-((2-(trimethylsilyl)ethoxy)methoxy)piperidine-1-carboxylate

A mixture of tert-butyl 4-(3-hydroxypropyl)-4-((2-(trimethylsilyl)ethoxy)methoxy)piperidine-1-carboxylate (0.72 g, 1.85 mmol, 1.0 equiv.), 4-bromo-3-methyl-phenol (380.21 mg, 2.03 mmol, 1.1 equiv.), and 2-(tributyl-phosphanylidene)acetonitrile (669.04 mg, 2.77 mmol, 1.5 equiv.) in toluene (20 mL) was degassed and purged with N₂ three times. The mixture was stirred at 125° C. for 58 hours under a N₂ atmosphere. The reaction mixture was concentrated under reduced pressure to give a residue, which was purified by flash silica gel chromatography (0-6% ethyl acetate/petroleum ether) to give tert-butyl 4-(3-(4-bromo-3-methylphenoxy)propyl)-4-((2-(trimethylsilyl)ethoxy)methoxy)piperidine-1-carboxylate (340 mg) as a yellow oil.

LC-MS (ESI): m/z=558.1 [M+H]⁺

¹H NMR (400 MHz, DMSO-d₆) δ=7.42 (d, J=8.8 Hz, 1H), 6.93 (d, J=2.8 Hz, 1H), 6.70 (dd, J=3.2, 8.8 Hz, 1H), 4.67 (s, 2H), 3.92 (t, J=6.4 Hz, 2H), 3.62-3.58 (m, 4H), 3.06 (s, 2H), 2.29 (s, 3H), 1.74-1.65 (m, 4H), 1.62-1.58 (m, 2H), 1.39 (s, 9H), 1.33 (d, J=4.4 Hz, 2H), 0.86-0.81 (m, 2H), 0.02 (s, 9H)

### Step 4: 4-(3-(4-bromo-3-methylphenoxy)propyl)piperidin-4-ol

A mixture of tert-butyl 4-(3-(4-bromo-3-methylphenoxy)propyl)-4-((2-(trimethylsilyl)ethoxy)methoxy)piperidine-1-carboxylate (340 mg, 608.64 mol, 1.0 equiv.) in HCl/dioxane (4M, 1 mL) and EtOAc (3 mL) was stirred at 25° C. for 2 hours. The reaction mixture was concentrated under reduced pressure to give 4-(3-(4-bromo-3-methylphenoxy)propyl)piperidin-4-ol (320 mg, crude, HCl) as a yellow oil, Step 5: ethyl 2-(4-(3-(4-bromo-3-methylphenoxy)propyl)-4-hydroxypiperidin-1-yl)acetate

To a mixture of 4-(3-(4-bromo-3-methylphenoxy)propyl)piperidin-4-ol (320 mg, 974.89 mol, 1.0 equiv.) and potassium carbonate (404.22 mg, 2.92 mmol, 3.0 equiv.) in DMF (2 mL) was added ethyl 2-bromoacetate (130.25 mg, 779.91 mol, 86.31 L, 0.8 equiv.). The mixture was stirred at 40° C. for 1 hour. The reaction mixture was filtered and filtered liquid was concentrated under reduced pressure to give a residue, which was purified by flash silica gel chromatography (0-57% ethyl acetate/petroleum ether) to give ethyl 2-(4-(3-(4-bromo-3-methylphenoxy)propyl)-4-hydroxypiperidin-1-yl)acetate (196 mg) as a yellow oil.

LC-MS (ESI): m/z=414.0 [M+H]⁺

¹H NMR (400 MHz, DMSO-d₆) δ=7.42 (d, J=8.8 Hz, 1H), 6.94 (d, J=2.8 Hz, 1H), 6.71 (dd, J=3.2, 8.8 Hz, 1H), 4.09-4.04 (m, 2H), 3.92 (t, J=6.4 Hz, 2H), 3.15 (s, 2H), 2.89 (s, 1H), 2.47-2.44 (m, 2H), 2.29 (s, 3H), 1.77-1.71 (m, 2H), 1.48-1.40 (m, 8H), 1.18 (t, J=7.2 Hz, 3H)

### Step 6: tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)-4-hydroxypiperidin-4-yl)propoxy)-2-methylphenyl)picolinate

A mixture of ethyl 2-(4-(3-(4-bromo-3-methylphenoxy)propyl)-4-hydroxypiperidin-1-yl)acetate (166 mg, 400.64 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (269.95 mg, 440.71 mol, 1.1 equiv.), potassium carbonate (166.11 mg, 1.20 mmol, 3.0 equiv.), and Ad₂nBuP Pd G₃ (58.36 mg, 80.13 mol, 0.2 equiv.) in dioxane (3 mL) and H₂O (0.3 mL) was degassed and purged with N₂ three times. The mixture was stirred at 100° C. for 1 hour under a N₂ atmosphere. The residue was purified by flash silica gel chromatography (0~98% ethyl acetate/petroleum ether) to give tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)-4-hydroxypiperidin-4-yl)propoxy)-2-methylphenyl)picolinate (260 mg) as a yellow solid Step 7: 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-4-hydroxypiperidin-1-yl)acetic acid

A mixture of tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)-4-hydroxypiperidin-4-yl)propoxy)-2-methylphenyl)picolinate (260 mg, 317.07 mol, 1.0 equiv.) and LiOH·H₂O (39.92 mg, 951.21 mol, 3.0 equiv.) in THF (4 mL) and H₂O (1 mL) was stirred at 35° C. for 2 hours under a N₂ atmosphere. The mixture was concentrated and diluted with H₂O (2 mL). The pH of the mixture was adjusted to pH=3 with 1 M HCl. The mixture was filtered, and the filter cake was concentrated to give 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-4-hydroxypiperidin-1-yl)acetic acid (142 mg, crude) as a yellow solid.

### Step 8: tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate

A mixture of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-4-hydroxypiperidin-1-yl)acetic acid (132 mg, 166.68 mol, 1.0 equiv.), 3-(6-amino-1-methyl-indazol-3-yl)piperidine-2,6-dione (47.35 mg, 183.34 mol, 1.1 equiv.), and EDCI (47.93 mg, 250.02 mol, 1.5 equiv.) in pyridine (2 mL) was stirred at 40° C. for 7 hours. The reaction mixture was partitioned between H₂O (10 mL) and DCM (30 mL). The organic phase was separated, washed with brine (30 mL), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate (0.2 g, crude) as a yellow oil.

### Step 9: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate (200 mg, 193.76 mol, 1.0 equiv.) in TFA (1 mL) and DCM (1 mL) was stirred at 40° C. for 2 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid (57.28 mg) as a yellow solid.

LC-MS (ESI): m/z=976.2 [M+H]⁺

¹H NMR (400 MHz, DMSO-d₆) δ=10.87 (s, 1H), 9.83 (s, 1H), 8.06-8.01 (m, 2H), 7.78 (d, J=8.0 Hz, 1H), 7.65-7.60 (m, 2H), 7.49-7.42 (m, 3H), 7.39-7.32 (m, 2H), 7.23 (dd, J=1.6, 8.8 Hz, 1H), 6.93 (dd, J=6.0, 8.4 Hz, 2H), 6.78 (d, J=2.0 Hz, 1H), 6.71 (d, J=8.4 Hz, 1H), 4.97 (s, 2H), 4.32 (dd, J=5.2, 9.6 Hz, 1H), 4.09 (s, 1H), 3.98-3.94 (m, 2H), 3.92 (s, 3H), 3.91-3.88 (m, 2H), 3.14 (s, 2H), 3.04-3.00 (m, 2H), 2.65-2.53 (m, 8H), 2.03 (s, 3H), 1.84-1.76 (m, 2H), 1.65-1.58 (m, 2H), 1.55-1.49 (m, 4H)

### Example 4: 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid (Compound 108)

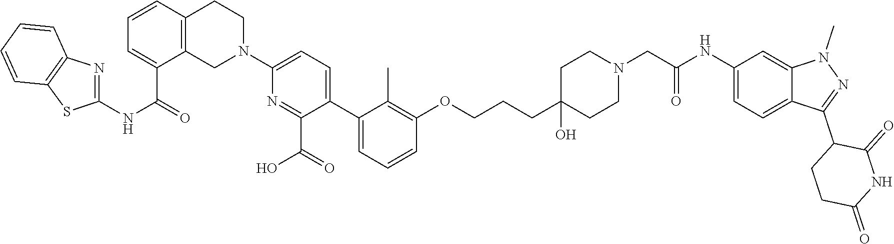 [SMILES: Cc1c(OCCCC2(O)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### Step 1: tert-butyl 4-allyl-4-((2-(trimethylsilyl)ethoxy)methoxy)piperidine-1-carboxylate

A mixture of tert-butyl 4-allyl-4-hydroxypiperidine-1-carboxylate (3.0 g, 12.43 mmol, 1.0 equiv.), TEA (6.29 g, 62.16 mmol, 8.65 mL, 5.0 equiv.), DMAP (303.74 mg, 2.49 mmol, 0.2 equiv.) in THF (20 mL) was degassed and purged with N₂ three times followed by the addition of (2-(chloromethoxy)ethyl)trimethylsilane (8.29 g, 49.73 mmol, 8.80 mL, 4.0 equiv.) at 0° C. The mixture was stirred at 40° C. for 16 hours under a N₂ atmosphere. The reaction mixture was filtered, and the filter liquid was concentrated under reduced pressure to give a residue, which was purified by flash silica gel chromatography (0-6% ethyl acetate/petroleum ether) to give tert-butyl 4-allyl-4-((2-(trimethylsilyl)ethoxy)methoxy)piperidine-1-carboxylate (2 g) as a colorless oil.

¹H NMR (400 MHz, DMSO-d₆) δ=5.83-5.71 (m, 1H), 5.07 (d, J=4.8 Hz, 1H), 5.04 (s, 1H), 4.71 (s, 2H), 3.64-3.55 (m, 4H), 3.05 (s, 2H), 2.29 (d, J=7.2 Hz, 2H), 1.66 (d, J=13.2 Hz, 2H), 1.38 (s, 11H), 0.89-0.81 (m, 2H), 0.00 (s, 9H)

### Step 2: tert-butyl 4-(3-hydroxypropyl)-4-((2-(trimethylsilyl)ethoxy)methoxy)piperidine-1-carboxylate

A mixture of tert-butyl 4-allyl-4-((2-(trimethylsilyl)ethoxy)methoxy)piperidine-1-carboxylate (1.2 g, 3.23 mmol, 1.0 equiv.) in THF (30 mL) was degassed and purged with N₂ three times followed by the addition of BH₃·THF (1 M, 3.23 mL, 1.0 equiv.) slowly at 0° C. The mixture was stirred at 25° C. for 12 hours. NaOH (129.18 mg, 3.23 mmol, 1.0 equiv.) was added at 0° C. followed by H₂O₂ (648.01 mg, 5.72 mmol, 549.16 L, 30% purity, 1.77 equiv.). The mixture was stirred at 25° C. for 5 hours under a N₂ atmosphere. The reaction mixture was quenched by the addition of sodium sulfite (saturated solution, 100 mL) at 25° C. and extracted with ethyl acetate (300 mL). The combined organic layers were washed with brine (150 mL), dried over sodium sulfate, and concentrated (below 30° C.) under reduced pressure to give tert-butyl 4-(3-hydroxypropyl)-4-((2-(trimethylsilyl)ethoxy)methoxy)piperidine-1-carboxylate (2 g) as a colorless oil.

¹H NMR (400 MHz, DMSO-d₆) δ=4.65 (s, 2H), 3.63-3.54 (m, 4H), 3.38-3.33 (m, 1H), 3.05 (s, 2H), 1.67 (d, J=13.2 Hz, 2H), 1.51-1.42 (m, 2H), 1.38 (s, 9H), 1.36-1.21 (m, 4H), 0.87-0.83 (m, 2H), 0.00 (s, 9H)

### Step 3: tert-butyl 4-(3-(3-bromo-2-methylphenoxy)propyl)-4-((2-(trimethylsilyl)ethoxy)methoxy)piperidine-1-carboxylate

A mixture of tert-butyl 4-(3-hydroxypropyl)-4-((2-(trimethylsilyl)ethoxy)methoxy)piperidine-1-carboxylate (0.72 g, 1.85 mmol, 1.0 equiv.), 3-bromo-2-methyl-phenol (380.21 mg, 2.03 mmol, 1.1 equiv.), and 2-(tributyl-phosphanylidene)acetonitrile (669.04 mg, 2.77 mmol, 1.5 equiv.) in toluene (20 mL) was degassed and purged with N₂ three times. The mixture was stirred at 125° C. for 58 hours under a N₂ atmosphere. The reaction mixture was concentrated under reduced pressure to give a residue, which was purified by flash silica gel chromatography (0-6% ethyl acetate/petroleum ether) to give tert-butyl 4-(3-(3-bromo-2-methylphenoxy)propyl)-4-((2-(trimethylsilyl)ethoxy)methoxy)piperidine-1-carboxylate (270 mg) as a yellow oil.

LC-MS (ESI): m/z=580.2 [M+Na]*.

### Step 4: 4-(3-(3-bromo-2-methylphenoxy)propyl)piperidin-4-ol

A mixture of tert-butyl 4-(3-(3-bromo-2-methylphenoxy)propyl)-4-((2-(trimethylsilyl)ethoxy)methoxy)piperidine-1-carboxylate (270 mg, 483.33 mol, 1.0 equiv.) in HCl/dioxane (4M, 1 mL) and EtOAc (3 mL) was stirred at 25° C. for 2 hours. The reaction mixture was concentrated under reduced pressure to give 4-(3-(3-bromo-2-methylphenoxy)propyl)piperidin-4-ol (260 mg, crude) as a yellow oil.

### Step 5: ethyl 2-(4-(3-(3-bromo-2-methylphenoxy)propyl)-4-hydroxypiperidin-1-yl)acetate

To a mixture of 4-(3-(3-bromo-2-methylphenoxy)propyl)piperidin-4-ol (260 mg, 792.09 mol, 1.0 equiv.) and potassium carbonate (328.42 mg, 2.38 mmol, 3.0 equiv.) in DMF (2 mL) was added ethyl 2-bromoacetate (105.82 mg, 633.68 mol, 70.13 L, 0.8 equiv.). The mixture was stirred at 40° C. for 1 hour under a N₂ atmosphere. The reaction mixture was filtered, and the filter liquid was concentrated under reduced pressure to give a residue, which was purified by flash silica gel chromatography (0-59% ethyl acetate/petroleum ether) to give ethyl 2-(4-(3-(3-bromo-2-methylphenoxy)propyl)-4-hydroxypiperidin-1-yl)acetate (113 mg) as a colorless oil.

LC-MS (ESI): m/z=416.1 [M+H]⁺

¹H NMR (400 MHz, DMSO-d₆) δ=7.14 (d, J=7.2 Hz, 1H), 7.09 (d, J=8.0 Hz, 1H), 6.95 (d, J=7.6 Hz, 1H), 4.10-4.05 (m, 2H), 3.97 (t, J=6.4 Hz, 2H), 3.15 (s, 2H), 2.89 (s, 1H), 2.43 (s, 2H), 2.24 (s, 3H), 1.80-1.74 (m, 2H), 1.53-1.39 (m, 8H), 1.18 (t, J=7.2 Hz, 3H)

### Step 6: tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-4-hydroxypiperidin-4-yl)propoxy)-2-methylphenyl)picolinate

A mixture ofethyl 2-(4-(3-(3-bromo-2-methylphenoxy)propyl)-4-hydroxypiperidin-1-yl)acetate (113 mg, 272.73 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (183.76 mg, 300.00 mol, 1.1 equiv.), potassium carbonate (113.08 mg, 818.18 mol, 3.0 equiv.), and Ad₂nBuP Pd G₃ (39.72 mg, 54.55 mol, 0.2 equiv.) in dioxane (3 mL) and H₂O (0.3 mL) was degassed and purged with N₂ three times. The mixture was stirred at 100° C. for 1 hour under a N₂ atmosphere. The reaction mixture was concentrated under reduced pressure to give a residue, which was purified by flash silica gel chromatography (0~99% ethyl acetate/petroleum ether) to give tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-4-hydroxypiperidin-4-yl)propoxy)-2-methylphenyl)picolinate (130 mg) as a yellow oil.

### Step 7: 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-4-hydroxypiperidin-1-yl)acetic acid

A mixture of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-4-hydroxypiperidin-4-yl)propoxy)-2-methylphenyl)picolinate (130 mg, 158.54 mol, 1.0 equiv.) LiOH·H₂O (19.96 mg, 475.61 mol, 3.0 equiv.) in THF (4 mL) and H₂O (1 mL) was stirred at 35° C. for 2 hours. The mixture was concentrated and diluted with H₂O (2 mL), and the pH of the mixture was adjusted to pH=3 with 1 M HCl. The mixture was filtered, and the filtrate cake was concentrated to give 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-4-hydroxypiperidin-1-yl)acetic acid (126 mg, crude) as a yellow solid.

### Step 8: tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate

A mixture of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-4-hydroxypiperidin-1-yl)acetic acid (116 mg, 146.47 mol, 1.0 equiv.), 3-(6-amino-1-methyl-indazol-3-yl)piperidine-2,6-dione (37.83 mg, 146.47 mol, 1.0 equiv.), and EDCI (42.12 mg, 219.71 mol, 1.5 equiv.) in pyridine (2 mL) was stirred at 40° C. for 50 hours. The reaction mixture was partitioned between H₂O (10 mL) and DCM (30 mL). The organic phase was separated, washed with brine (30 mL), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate (0.19 g, crude) as a yellow solid.

LC-MS (ESI): m/z=516.9 [M/2+H]⁺.

### Step 9: 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinate (190 mg, 184.07 mol, 1.0 equiv.) in DCM (1 mL) and TFA (1 mL) was stirred at 40° C. for 2 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)-4-hydroxypiperidin-4-yl)propoxy)-2-methylphenyl)picolinic acid (53.30 mg) as a yellow solid.

LC-MS (ESI): m/z=976.3 [M+H]⁺

¹H NMR (400 MHz, DMSO-d₆) δ=10.87 (s, 1H), 9.86-9.82 (m, 1H), 8.07-8.00 (m, 2H), 7.78 (d, J=8.4 Hz, 1H), 7.62 (d, J=8.4 Hz, 2H), 7.49-7.42 (m, 3H), 7.39-7.32 (m, 2H), 7.22 (dd, J=1.6, 8.8 Hz, 1H), 7.12-7.06 (m, 1H), 6.96 (d, J=8.8 Hz, 1H), 6.88 (d, J=8.4 Hz, 1H), 6.63 (d, J=7.2 Hz, 1H), 4.98 (s, 2H), 4.32 (dd, J=5.2, 9.6 Hz, 1H), 4.12-4.06 (m, 1H), 3.97 (t, J=5.6 Hz, 2H), 3.94-3.90 (m, 5H), 3.14 (s, 2H), 3.03 (t, J=5.6 Hz, 2H), 2.65-2.55 (m, 4H), 2.52 (s, 4H), 1.91 (s, 3H), 1.86-1.79 (m, 2H), 1.62-1.50 (m, 6H)

### Example 5: 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (Compound

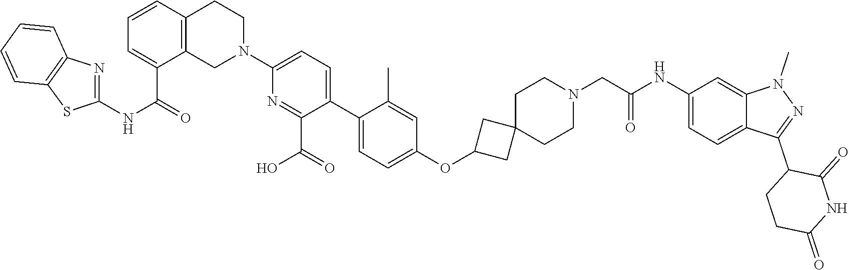 [SMILES: Cc1cc(OC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O]

### Step 1: tert-butyl 2-(4-bromo-3-methyl-phenoxy)-7-azaspiro[3.5]nonane-7-carboxylate

A mixture of tert-butyl 2-hydroxy-7-azaspiro[3.5]nonane-7-carboxylate (1 g, 4.14 mmol, 1.0 equiv.), 4-bromo-3-methyl-phenol (852.53 mg, 4.56 mmol, 1.1 equiv.), 2-(tributyl-phosphanylidene)acetonitrile (2.00 g, 8.29 mmol, 2.0 equiv.) in toluene (50 mL) was degassed and purged with N₂ three times. The mixture was stirred at 120° C. for 16 hours under a N₂ atmosphere. The reaction mixture was concentrated under reduced pressure to remove solvent. The residue was purified by flash silica gel chromatography (0-8% ethyl acetate/petroleum ether) to give tert-butyl 2-(4-bromo-3-methyl-phenoxy)-7-azaspiro[3.5]nonane-7-carboxylate (2.0 g) as a yellow oil.

LC-MS (ESI): m/z=354.1 [M−56+H]⁺

### Step 2: 2-(4-bromo-3-methyl-phenoxy)-7-azaspiro[3.5]nonane

A solution of tert-butyl 2-(4-bromo-3-methyl-phenoxy)-7-azaspiro[3.5]nonane-7-carboxylate (2.0 g, 4.87 mmol, 1.0 equiv.) in HCl/dioxane (30 mL) was stirred at 25° C. for 1 hour. The reaction mixture was concentrated under reduced pressure to remove dioxane. The reaction mixture was washed with petroleum ether (24 mL) and ethyl acetate (8 mL) and then filtered to give 2-(4-bromo-3-methyl-phenoxy)-7-azaspiro[3.5]nonane (944 mg) as a white solid.

LC-MS (ESI): m/z=310.0 [M+H]⁺

### Step 3: ethyl 2-[2-(4-bromo-3-methyl-phenoxy)-7-azaspiro[3.5]nonan-7-yl]acetate

To a solution of 2-(4-bromo-3-methyl-phenoxy)-7-azaspiro[3.5]nonane (944 mg, 3.04 mmol, 1.0 equiv.) and ethyl 2-bromoacetate (508.17 mg, 3.04 mmol, 336.76 μL, 1.0 equiv.) in DMF (10 mL) was added potassium carbonate (1.26 g, 9.13 mmol, 3.0 equiv.). The mixture was stirred at 40° C. for 1 hour. The residue was diluted with H₂O (50 mL) and extracted with ethyl acetate (150 mL). The combined organic layers were washed with H₂O (500 mL), dried over sodium sulfate, filtered, and concentrated under reduced pressure to give ethyl 2-[2-(4-bromo-3-methyl-phenoxy)-7-azaspiro[3.5]nonan-7-yl]acetate (1.01 g) as a yellow oil.

LC-MS (ESI): m/z=396.1 [M+H]⁺

### Step 4: tert-butyl 6-[8-(1,3-benzothiazol-2-ylcarbamoyl)-3,4-dihydro-1H-isoquinolin-2-yl]-3-[4-[[7-(2-ethoxy-2-oxo-ethyl)-7-azaspiro[3.5]nonan-2-yl]oxy]-2-methyl-phenyl]pyridine-2-carboxylate

A mixture of ethyl 2-[2-(4-bromo-3-methyl-phenoxy)-7-azaspiro[3.5]nonan-7-yl]acetate (500 mg, 1.26 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (927.36 mg, 1.51 mmol, 1.2 equiv.), KF (219.89 mg, 3.78 mmol, 88.66 μL, 3.0 equiv.), and Ad₂nBup Pd G₃ (183.76 mg, 252.32 mol, 0.2 equiv.) in dioxane (10 mL) and H₂O (2 mL) was degassed and purged with N₂ three times. The mixture was stirred at 100° C. for 16 hours under a N₂ atmosphere. The reaction mixture was concentrated under reduced pressure to remove the solvent. The residue was purified by flash silica gel chromatography (0-51% ethyl acetate/petroleum ether) to give tert-butyl 6-[8-(1,3-benzothiazol-2-ylcarbamoyl)-3,4-dihydro-1H-isoquinolin-2-yl]-3-[4-[[7-(2-ethoxy-2-oxo-ethyl)-7-azaspiro[3.5]nonan-2-yl]oxy]-2-methyl-phenyl]pyridine-2-carboxylate (692 mg) as a yellow solid.

LC-MS (ESI): m/z=802.3 [M+H]⁺

¹H NMR (400 MHz, DMSO-d₆) δ=12.87 (s, 1H), 8.03 (d, J=7.6 Hz, 1H), 7.78 (d, J=7.6 Hz, 1H), 7.60 (d, J=7.6 Hz, 1H), 7.56-7.42 (m, 3H), 7.36 (d, J=7.2, 11.6 Hz, 2H), 6.93 (d, J=8.8 Hz, 1H), 6.85 (d, J=8.4 Hz, 1H), 6.72 (d, J=0.8 Hz, 1H), 6.63 (dd, J=2.0, 8.4 Hz, 1H), 4.96 (s, 2H), 4.07 (q, J=7.2 Hz, 2H), 3.87 (t, J=5.6 Hz, 2H), 3.61 (t, J=6.4 Hz, 5H), 3.15 (s, 2H), 2.00 (s, 3H), 1.79-1.74 (m, 6H), 1.60-1.51 (m, 4H), 1.18 (t, J=7.2 Hz, 3H), 1.02 (s, 9H)

### Step 5: 2-[2-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-7-azaspiro[3.5]nonan-7-yl]acetic acid

To a solution of tert-butyl 6-[8-(1,3-benzothiazol-2-ylcarbamoyl)-3,4-dihydro-1H-isoquinolin-2-yl]-3-[4-[[7-(2-ethoxy-2-oxo-ethyl)-7-azaspiro[3.5]nonan-2-yl]oxy]-2-methyl-phenyl]pyridine-2-carboxylate (692 mg, 862.85 mol, 1.0 equiv.) in H₂O (10 mL) and THE (10 mL) was added LiOH·H₂O (144.83 mg, 3.45 mmol, 4.0 equiv.). The mixture was stirred at 40° C. for 2 hours. The reaction mixture was concentrated under reduced pressure to remove H₂O and THF. H₂O (30 mL) was added to the mixture, and the pH was adjusted to pH=3~4 with HCl (1 N). The resulting mixture was filtered to give 2-[2-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-7-azaspiro[3.5]nonan-7-yl]acetic acid (531 mg) as a yellow solid.

LC-MS (ESI): m/z=774.3 [M+H]⁺

### Step 6: tert-butyl 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate

To a solution of 2-[2-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-7-azaspiro[3.5]nonan-7-yl]acetic acid (200 mg, 258.42 mol, 1.0 equiv.) and 3-(6-amino-1-methyl-indazol-3-yl)piperidine-2,6-dione (80.09 mg, 310.10 mol, 1.2 equiv.) in pyridine (5 mL) was added EDCI (74.31 mg, 387.63 mol, 1.5 equiv.). The mixture was stirred at 40° C. for 1 hour. The reaction mixture was concentrated under reduced pressure to remove the solvent. The residue was diluted with H₂O (50 mL) and extracted with DCM:MeOH (10:1) (150 mL). The combined organic layers were washed with brine (50 mL), dried over sodium sulfate, filtered, and concentrated under reduced pressure to give a residue, which was purified by flash silica gel chromatography (0~20% DCM/MeOH) to give tert-butyl 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (284 mg) as a yellow oil.

LC-MS (ESI): m/z=1014.4 [M+H]⁺

### Step 7: 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (284 mg, 221.22 mol, 1.0 equiv.) in DCM (4 mL) and TFA (8 mL) was stirred at 40° C. for 2 hours. The reaction mixture was concentrated under reduced pressure to remove DCM and TFA to give 6-[8-(1,3-benzothiazol-2-ylcarbamoyl)-3,4-dihydro-1H-isoquinolin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (74.14 mg) as a white solid.

LC-MS (ESI): m/z=959.3 [M+H]⁺

¹H NMR (400 MHz, DMSO-d₆) δ=13.22-12.35 (m, 2H), 10.91 (s, 1H), 9.86 (s, 1H), 8.13-8.00 (m, 2H), 7.80 (d, J=8.0 Hz, 1H), 7.69-7.60 (m, 2H), 7.50-7.42 (m, 3H), 7.41-7.34 (m, 2H), 7.23 (d, J=9.6 Hz, 1H), 6.93 (dd, J=8.8, 14.4 Hz, 2H), 6.71 (d, J=2.0 Hz, 1H), 6.63 (dd, J=2.0, 8.4 Hz, 1H), 4.98 (s, 2H), 4.72 (s, 1H), 4.33 (d, J=4.8 Hz, 1H), 3.98-3.86 (m, 5H), 3.13 (s, 2H), 3.03 (t, J=5.6 Hz, 2H), 2.65-2.61 (m, 4H), 2.43-2.41 (m, 4H), 2.22-2.14 (m, 2H), 2.03 (s, 3H), 1.83-1.75 (m, 2H), 1.73-1.63 (m, 4H).

### Example 6: 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (Compound 113)

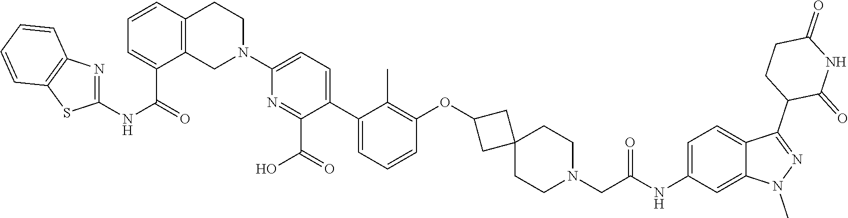 [SMILES: Cc1c(OC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### Step 1: tert-butyl 2-(3-bromo-2-methyl-phenoxy)-7-azaspiro[3.5]nonane-7-carboxylate

A mixture of tert-butyl 2-hydroxy-7-azaspiro[3.5]nonane-7-carboxylate (2 g, 8.29 mmol, 1.0 equiv.), 3-bromo-2-methyl-phenol (1.71 g, 9.12 mmol, 1.1 equiv.), and 2-(tributyl-phosphanylidene)acetonitrile (3.00 g, 12.43 mmol, 1.5 equiv.) in toluene (20 mL) was degassed and purged with N₂ three times. The mixture was stirred at 125° C. for 12 hours under a N₂ atmosphere. The reaction mixture was concentrated under reduced pressure to give a residue, which was purified by flash silica gel chromatography (0-5% ethyl acetate/petroleum ether) to give tert-butyl 2-(3-bromo-2-methyl-phenoxy)-7-azaspiro[3.5]nonane-7-carboxylate (3.2 g, crude) as a white solid.

¹H NMR (400 MHz, DMSO-d₆) δ=7.16 (d, J=8.0 Hz, 1H), 7.07 (t, J=8.0 Hz, 1H), 6.78 (d, J=8.0 Hz, 1H), 4.76 (s, 1H), 3.28 (d, J=5.6 Hz, 2H), 3.24-3.19 (m, 2H), 2.44-2.39 (m, 2H), 2.24 (s, 3H), 1.84-1.77 (m, 2H), 1.52 (dd, J=5.6, 15.2 Hz, 4H), 1.39 (s, 9H)

### Step 2: 2-(3-bromo-2-methyl-phenoxy)-7-azaspiro[3.5]nonane

A solution of tert-butyl 2-(3-bromo-2-methyl-phenoxy)-7-azaspiro[3.5]nonane-7-carboxylate (3.2 g, 7.80 mmol, 1.0 equiv.) in HCl/dioxane (4 M, 20 mL) and dioxane (10 mL) was stirred at 25° C. for 40 minutes. The reaction mixture was filtered and concentrated under reduced pressure to give 2-(3-bromo-2-methyl-phenoxy)-7-azaspiro[3.5]nonane (2.3 g, crude) as a white solid.

### Step 3: ethyl 2-[2-(3-bromo-2-methyl-phenoxy)-7-azaspiro[3.5]nonan-7-yl]acetate

To a solution of 2-(3-bromo-2-methyl-phenoxy)-7-azaspiro[3.5]nonane (1.1 g, 3.55 mmol, 1.0 equiv.) and ethyl 2-bromoacetate (592.15 mg, 3.55 mmol, 392.41 μL, 1.0 equiv.) in DMF (5 mL) was added potassium carbonate (1.47 g, 10.64 mmol, 3.0 equiv.). The mixture was stirred at 40° C. for 1 hour. The reaction mixture was partitioned between EtOAc (30 mL) and water (20 mL). The organic phase was separated, washed with water (120 mL), dried over sodium sulfate, filtered, and concentrated under reduced pressure to give ethyl 2-[2-(3-bromo-2-methyl-phenoxy)-7-azaspiro[3.5]nonan-7-yl]acetate (1.2 g, crude) as a yellow oil.

¹H NMR (400 MHz, DMSO-d₆) δ=7.13 (d, J=8.0 Hz, 1H), 7.06 (t, J=8.0 Hz, 1H), 6.77 (d, J=8.0 Hz, 1H), 4.72 (s, 1H), 4.06 (q, J=7.2 Hz, 2H), 3.14 (s, 2H), 2.43 (s, 2H), 2.39-2.33 (m, 4H), 2.22 (s, 3H), 1.78-1.72 (m, 2H), 1.56 (td, J=5.6, 13.4 Hz, 4H), 1.17 (t, J=7.2 Hz, 3H)

### Step 4: tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]oxy]-2-methyl-phenyl]pyridine-2-carboxylate

A mixture of ethyl 2-[2-(3-bromo-2-methyl-phenoxy)-7-azaspiro[3.5]nonan-7-yl]acetate (250 mg, 630.81 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (425.04 mg, 693.89 mol, 1.1 equiv.), KF (109.94 mg, 1.89 mmol, 44.33 μL, 3.0 equiv.), and Ad₂nBuP Pd G₃ (91.88 mg, 126.16 mol, 0.2 equiv.) in dioxane (10 mL) and H₂O (1 mL) was degassed and purged with N₂ three times. The mixture was stirred at 100° C. for 1 hour under a N₂ atmosphere. The reaction mixture was partitioned between ethyl acetate (30 mL) and water (20 mL). The organic phase was separated, washed with brine (20 mL), dried over sodium sulfate, filtered, and concentrated under reduced pressure to give a residue, which was purified by flash silica gel chromatography (0-60% ethyl acetate/petroleum ether) to give tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]oxy]-2-methyl-phenyl]pyridine-2-carboxylate (320 mg) as a yellow solid.

### Step 5: 2-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-7-azaspiro[3.5]nonan-7-yl]acetic acid

To a solution of tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]oxy]-2-methyl-phenyl]pyridine-2-carboxylate (320 mg, 399.01 mol, 1.0 equiv.) in THF (3 mL) and H₂O (0.8 mL) was added LiOH·H₂O (50.23 mg, 1.20 mmol, 3.0 equiv.). The mixture was stirred at 40° C. for 1 hour. The reaction mixture was acidified to pH=3-4 with citric acid and partitioned between DCM (20 mL) and water (15 mL). The organic phase was separated, washed with brine (15 mL), dried over sodium sulfate, filtered, and concentrated under reduced pressure to give 2-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-7-azaspiro[3.5]nonan-7-yl]acetic acid (350 mg, crude) as a yellow solid.

### Step 6: tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate

To a solution of 2-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-7-azaspiro[3.5]nonan-7-yl]acetic acid (350 mg, 452.23 mol, 1.0 equiv.) and 3-(6-amino-1-methyl-indazol-3-yl)piperidine-2,6-dione (140.16 mg, 542.68 mol, 1.2 equiv.) in pyridine (4 mL) was added EDCI (173.39 mg, 904.47 mol, 2.0 equiv.). The mixture was stirred at 60° C. for 1 hour. The reaction mixture was concentrated under reduced pressure to remove the solvent. The residue was diluted with DCM (40 mL) and extracted with water (120 mL). The combined organic layers were washed with brine (30 mL), dried over sodium sulfate, filtered, and concentrated under reduced pressure to give tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (300 mg, crude) as a yellow oil.

### Step 7: 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (300 mg, 295.80 mol, 1.0 equiv.) in TFA (3 mL) and DCM (1.5 mL) was stirred at 25° C. for 12 hours. The reaction mixture was concentrated under reduced pressure to remove to give a residue, which was purified by reversed-phase HPLC to give 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (207.78 mg) as a white solid.

LC-MS (ESI): m/z=958.9 [M+H]⁺

¹H NMR (400 MHz, DMSO-d₆) δ=12.93-12.84 (m, 1H), 12.61 (s, 1H), 10.91 (s, 1H), 8.03 (d, J=7.2 Hz, 2H), 7.79 (d, J=8.4 Hz, 1H), 7.68-7.59 (m, 2H), 7.49-7.43 (m, 3H), 7.37 (dd, J=7.6, 9.6 Hz, 2H), 7.24-7.15 (m, 1H), 7.07 (t, J=8.0 Hz, 1H), 6.97 (d, J=8.8 Hz, 1H), 6.72 (d, J=8.0 Hz, 1H), 6.63 (d, J=8.0 Hz, 1H), 5.01-4.94 (m, 2H), 4.77-4.67 (m, 1H), 4.33 (dd, J=4.8, 9.6 Hz, 1H), 3.93 (s, 5H), 3.39 (s, 2H), 3.02 (s, 2H), 2.57-2.56 (m, 4H), 2.36 (d, J=3.6 Hz, 3H), 2.23-2.10 (m, 4H), 1.89 (s, 3H), 1.87-1.78 (m, 3H), 1.75-1.68 (m, 2H)

### Example 7: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinic acid (Compound 129)

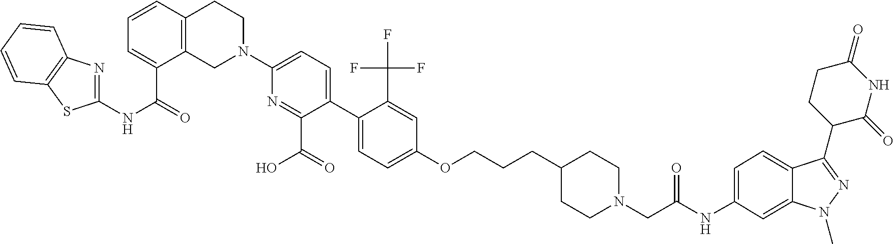 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(C(F)(F)F)c4)CC3)cc21]

### Step 1: tert-butyl 4-(3-(4-bromo-3-(trifluoromethyl)phenoxy)propyl)piperidine-1-carboxylate

A mixture of 4-bromo-3-(trifluoromethyl)phenol (500.00 mg, 2.07 mmol, 1 equiv.), tert-butyl 4-(3-bromopropyl)piperidine-1-carboxylate (762.40 mg, 2.49 mmol, 1.2 equiv.), and potassium carbonate (860.18 mg, 6.22 mmol, 3 equiv.) in DMF (12 mL) was degassed and purged with N₂ three times. The mixture was stirred at 60° C. for 3 hours under a N₂ atmosphere. The combined organic layers were diluted with DCM (10 mL), washed with H₂O (30 mL), and extracted with DCM (30 mL). The organic layer was filtered and concentrated under reduced pressure to afford tert-butyl 4-(3-(4-bromo-3-(trifluoromethyl)phenoxy)propyl)piperidine-1-carboxylate (1.0 g, crude) as a yellow oil.

LC-MS (ESI): m/z=410.1 [M−56+H]⁺

### Step 2: 4-(3-(4-bromo-3-(trifluoromethyl)phenoxy)propyl)piperidine

A solution of tert-butyl 4-(3-(4-bromo-3-(trifluoromethyl)phenoxy)propyl)piperidine-1-carboxylate (900 mg, 1.93 mmol, 1 equiv.) in HCl/dioxane (10 mL) was stirred at 25° C. for 2 hours. The combined organic layers were filtered and concentrated under reduced pressure to afford 4-(3-(4-bromo-3-(trifluoromethyl)phenoxy)propyl)piperidine (900 mg, crude, HCl) as a white solid.

LC-MS (ESI): m/z=366.1 [M+H]⁺

¹H NMR (400 MHz, DMSO-d₆) δ=7.95 (s, 1H), 7.75 (d, J=8.8 Hz, 1H), 7.31 (d, J=3.2 Hz, 1H), 7.18 (dd, J=2.8, 8.8 Hz, 1H), 4.04 (t, J=6.4 Hz, 2H), 3.21 (d, J=12.4 Hz, 2H), 1.87-1.65 (m, 5H), 1.58-1.49 (m, 1H), 1.41-1.24 (m, 5H).

### Step 3: ethyl 2-(4-(3-(4-bromo-3-(trifluoromethyl)phenoxy)propyl)piperidin-1-yl)acetate

A mixture of ethyl 2-bromoacetate (373.25 mg, 2.24 mmol, 247.35 μL, 1 equiv.), 4-(3-(4-bromo-3-(trifluoromethyl)phenoxy)propyl)piperidine (900.00 mg, 2.24 mmol, 1 equiv., HCl), TEA (1.13 g, 11.18 mmol, 1.56 mL, 5 equiv.) in acetone (8 mL) was stirred at 25° C. for 2 hours under a N₂ atmosphere. The combined organic layers were filtered and concentrated under reduced pressure to give a residue, which was purified by flash silica gel chromatography (0~30% ethyl acetate/petroleum ether) to afford ethyl 2-(4-(3-(4-bromo-3-(trifluoromethyl)phenoxy)propyl)piperidin-1-yl)acetate (900 mg) as a yellow oil.

LC-MS (ESI): m/z=452.2 [M+H]⁺

¹H NMR (400 MHz, DMSO-d₆) δ=7.74 (d, J=8.8 Hz, 1H), 7.29 (d, J=2.8 Hz, 1H), 7.17 (dd, J=2.8, 8.8 Hz, 1H), 3.14 (s, 2H), 2.79 (d, J=11.2 Hz, 2H), 2.10 (t, J=10.4 Hz, 2H), 1.75-1.67 (m, 2H), 1.61 (d, J=12.0 Hz, 2H), 1.32-1.29 (m, 1H), 1.19-1.12 (m, 5H).

### Step 4: tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-(trifluoromethyl)phenyl)picolinate

A mixture of ethyl 2-(4-(3-(4-bromo-3-(trifluoromethyl)phenoxy)propyl)piperidin-1-yl)acetate (250 mg, 552.72 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (406.28 mg, 663.27 mol, 1.2 equiv.), potassium carbonate (1.5 M, 552.72 μL, 1.5 equiv.), and Ad₂nBup Pd G₃ (23.67 mg, 110.54 mol, 0.2 equiv.) in dioxane (8 mL) was degassed and purged with N₂ three times. The mixture was stirred at 100° C. for 2 hours under a N₂ atmosphere in a microwave reactor. The combined organic layers were filtered and concentrated under reduced pressure to give a residue, which was purified by flash silica gel chromatography (0-40% ethyl acetate/petroleum ether) to afford tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-(trifluoromethyl)phenyl)picolinate (270 mg) as a yellow solid.

LC-MS (ESI): m/z=858.7 [M+H]⁺

¹H NMR (400 MHz, DMSO-d6) δ=12.93-12.78 (m, 1H), 8.02 (d, J=8.0 Hz, 1H), 7.78 (d, J=8.0 Hz, 1H), 7.60 (d, J=7.6 Hz, 1H), 7.48-7.40 (m, 3H), 7.36 (dd, J=7.2, 13.6 Hz, 2H), 7.24-7.18 (m, 2H), 7.16-7.08 (m, 1H), 6.95 (d, J=8.8 Hz, 1H), 5.08-4.93 (m, 2H), 4.10-4.02 (m, 4H), 3.88 (t, J=5.6 Hz, 2H), 3.16 (s, 2H), 3.03 (t, J=5.6 Hz, 2H), 2.80 (d, J=10.4 Hz, 2H), 2.11 (t, J=10.4 Hz, 2H), 1.76-1.69 (m, 2H), 1.62 (d, J=11.2 Hz, 2H), 1.37-1.30 (m, 2H), 1.28-1.20 (m, 1H), 1.20-1.14 (m, 5H), 1.02 (s, 9H).

### Step 5: 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)piperidin-1-yl)acetic acid

A mixture of tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-(trifluoromethyl)phenyl)picolinate (270 mg, 314.69 μmol, 1 equiv.) and LiOH·H₂O (39.62 mg, 944.08 μmol, 3 equiv.) in THF and H₂O (1.5 mL) was stirred at 25° C. for 2 hours. The combined organic layers were filtered and concentrated under reduced pressure to give a residue, which was diluted with DCM (6 mL) and washed with H₂O (9 mL). The mixture was extracted with DCM (30 mL), filtered, and concentrated under reduced pressure to afford 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)piperidin-1-yl)acetic acid (270 mg, crude) as a yellow solid.

### Step 6: tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinate

A mixture of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)piperidin-1-yl)acetic acid (240 mg, 289.18 μmol, 1 equiv.), 3-(6-amino-1-methyl-indazol-3-yl)piperidine-2,6-dione (89.63 mg, 347.02 μmol, 1.2 equiv.) and EDCI (83.15 mg, 433.77 μmol, 1.5 equiv.) in pyridine (5 mL) was degassed and purged with N₂ three times. The mixture was stirred at 25° C. for 2 hours under a N₂ atmosphere. The reaction mixture was concentrated under reduced pressure to remove pyridine. The residue was diluted with DCM (10 mL). The combined organic layers were washed with H₂O (30 mL), extracted with DCM (45 mL), filtered, and concentrated under reduced pressure to afford tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinate (300 mg) as a brown oil.

### Step 7: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinic acid

A solution of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinate (300 mg, 280.33 mol, 1 equiv.) in TFA (1.5 mL) and DCM (1.5 mL) was stirred at 25° C. for 16 hours. The combined organic layers were filtered and concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-(trifluoromethyl)phenyl)picolinic acid (79.85 mg) as a yellow solid.

LC-MS (ESI): m/z=508.0 [M/2+H]⁺.

¹H NMR (400 MHz, DMSO-d₆) δ=10.88 (s, 1H), 9.84 (s, 1H), 8.14 (s, 1H), 8.06-7.99 (m, 2H), 7.79 (d, J=7.6 Hz, 1H), 7.63 (d, J=6.4 Hz, 2H), 7.50-7.42 (m, 3H), 7.41-7.31 (m, 2H), 7.24-7.15 (m, 4H), 6.99 (d, J=8.4 Hz, 1H), 5.08-4.92 (m, 2H), 4.36-4.27 (m, 1H), 4.09-4.02 (m, 2H), 3.96-3.88 (m, 5H), 3.15 (s, 2H), 3.06-3.00 (m, 2H), 2.90 (d, J=9.6 Hz, 2H), 2.68-2.61 (m, 2H), 2.37-2.29 (m, 2H), 2.22-2.13 (m, 3H), 1.80-1.67 (m, 4H), 1.40 (d, J=3.2 Hz, 2H), 1.29 (s, 2H).

### Example 8: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid (Compound 131)

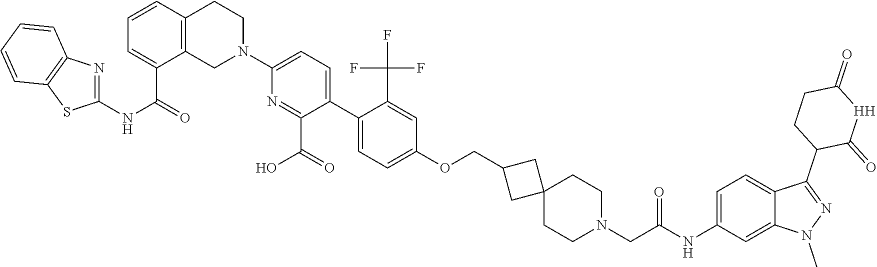

### Step 1: tert-butyl 2-((4-bromo-3-(trifluoromethyl)phenoxy)methyl)-7-azaspiro[3.5]nonane-7-carboxylate

A mixture of tert-butyl 2-(hydroxymethyl)-7-azaspiro[3.5]nonane-7-carboxylate (600 mg, 2.35 mmol, 1 equiv.), 4-bromo-3-(trifluoromethyl)phenol (566.29 mg, 2.35 mmol, 1 equiv.), and 2-(tributyl-phosphanylidene)acetonitrile (850.66 mg, 3.52 mmol, 1.5 equiv.) in toluene (12 mL) was degassed and purged with N₂ three times. The mixture was stirred at 120° C. for 12 hours under a N₂ atmosphere. The combined organic layers were filtered and concentrated under reduced pressure to give a residue, which was purified by flash silica gel chromatography (0~20% ethyl acetate/petroleum ether) to afford tert-butyl 2-((4-bromo-3-(trifluoromethyl)phenoxy)methyl)-7-azaspiro[3.5]nonane-7-carboxylate (1 g) as a yellow oil.

LC-MS (ESI): m/z=422.1 [M−56+H]⁺.

¹H NMR (400 MHz, DMSO-d₆) δ=7.74 (d, J=8.8 Hz, 1H), 7.30 (s, 1H), 7.17 (d, J=8.8 Hz, 1H), 4.02 (d, J=6.0 Hz, 2H), 3.26 (s, 2H), 3.18 (s, 2H), 2.67 (td, J=7.6, 14.8 Hz, 1H), 1.91 (t, J=10.4 Hz, 2H), 1.61 (t, J=10.4 Hz, 2H), 1.51 (s, 2H), 1.42 (s, 2H), 1.38 (s, 9H)

### Step 2: 2-((4-bromo-3-(trifluoromethyl)phenoxy)methyl)-7-azaspiro[3.5]nonane

A mixture of tert-butyl 2-((4-bromo-3-(trifluoromethyl)phenoxy)methyl)-7-azaspiro[3.5]nonane-7-carboxylate (1 g, 2.09 mmol, 1 equiv.) in HCl/dioxane (10 mL) was stirred at 25° C. for 1 hour. The reaction mixture was filtered and concentrated under reduced pressure to afford 2-((4-bromo-3-(trifluoromethyl)phenoxy)methyl)-7-azaspiro[3.5]nonane (800 mg) as a white solid.

### Step 3: ethyl 2-(2-((4-bromo-3-(trifluoromethyl)phenoxy)methyl)-7-azaspiro[3.5]nonan-7-yl)acetate

A mixture of 2-((4-bromo-3-(trifluoromethyl)phenoxy)methyl)-7-azaspiro[3.5]nonane (750 mg, 1.81 mmol, 1 equiv. HCl), ethyl 2-bromoacetate (302.04 mg, 1.81 mmol, 200.16 μL, 1 equiv.), and TEA (915.05 mg, 9.04 mmol, 1.26 mL, 5 equiv.) in MeCN (10 mL) was stirred at 40° C. for 1 hour. The reaction mixture was filtered and concentrated under reduced pressure to give a residue, which was purified by flash silica gel chromatography 0~20% ethyl acetate/petroleum ether) to afford ethyl 2-(2-((4-bromo-3-(trifluoromethyl)phenoxy)methyl)-7-azaspiro[3.5]nonan-7-yl)acetate (800 mg) as a yellow oil.

LC-MS (ESI): m/z=464.2 [M+H]⁺

¹H NMR (400 MHz, DMSO-d₆) δ=7.73 (d, J=8.8 Hz, 1H), 7.29 (d, J=2.8 Hz, 1H), 7.16 (dd, J=2.8, 8.8 Hz, 1H), 4.06 (q, J=6.8 Hz, 2H), 4.00 (d, J=6.4 Hz, 2H), 3.12 (s, 2H), 2.68-2.59 (m, 1H), 2.40 (s, 2H), 2.32 (s, 2H), 1.89-1.83 (m, 2H), 1.59-1.53 (m, 4H), 1.48 (t, J=5.2 Hz, 2H), 1.17 (t, J=6.8 Hz, 3H)

### Step 4: tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-(trifluoromethyl)phenyl)picolinate

A mixture of ethyl 2-(2-((4-bromo-3-(trifluoromethyl)phenoxy)methyl)-7-azaspiro[3.5]nonan-7-yl)acetate (250 mg, 538.43 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (395.77 mg, 646.11 mol, 1.2 equiv.), Ad₂nBup Pd G₃ (78.42 mg, 107.69 mol, 0.2 equiv.), and potassium carbonate (1.5 M, 358.95 μL, 1 equiv.) in dioxane (8 mL) was degassed and purged with N₂ three times. The mixture was stirred at 100° C. for 1 hour under a N₂ atmosphere at microwave. The reaction mixture was filtered and concentrated under reduced pressure to give a residue, which was purified by flash silica gel chromatography (0~40% ethyl acetate/petroleum ether) to afford tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-(trifluoromethyl)phenyl)picolinate (260 mg) as a yellow solid.

LC-MS (ESI): m/z=407.8 [M/2+H]⁺.

¹H NMR (400 MHz, DMSO-d₆) δ=12.96-12.77 (m, 1H), 8.02 (d, J=8.0 Hz, 1H), 7.78 (d, J=8.0 Hz, 1H), 7.60 (d, J=7.2 Hz, 1H), 7.47-7.33 (m, 5H), 7.23-7.17 (m, 2H), 7.14-7.09 (m, 1H), 6.95 (d, J=8.4 Hz, 1H), 5.00 (q, J=17.2 Hz, 2H), 4.05 (dd, J=6.8, 12.4 Hz, 4H), 3.88 (t, J=5.6 Hz, 2H), 3.13 (s, 2H), 3.03 (t, J=5.6 Hz, 2H), 2.68-2.63 (m, 1H), 2.42 (s, 2H), 2.33 (s, 2H), 1.87 (t, J=10.8 Hz, 2H), 1.60-1.54 (m, 4H), 1.49 (t, J=4.4 Hz, 2H), 1.17 (t, J=7.2 Hz, 3H), 1.02 (s, 9H)

### Step 5: 2-(2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)methyl)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-(trifluoromethyl)phenyl)picolinate (260 mg, 298.85 mol, 1 equiv.) and LiOH H₂O (37.62 mg, 896.56 mol, 3 equiv.) in THE (3 mL) and H₂O (1 mL) was stirred at 25° C. for 16 hours. The reaction mixture was diluted with H₂O (5 mL) and concentrated as a turbid liquid. The pH of the turbid liquid was adjusted to pH=3 with 1 N HCl (5 mL). The residue was extracted with DCM/MEOH (10/1) (30 mL) and the combined organic layers were filtered and concentrated to afford 2-(2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)methyl)-7-azaspiro[3.5]nonan-7-yl)acetic acid (250 mg) as a yellow solid.

### Step 6: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate

A mixture of 2-(2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)methyl)-7-azaspiro[3.5]nonan-7-yl)acetic acid (150 mg, 178.16 mol, 1 equiv.), 3-(6-amino-1-methyl-indazol-3-yl)piperidine-2,6-dione (55.22 mg, 213.79 mol, 1.2 equiv.), and EDCI (51.23 mg, 267.24 mol, 1.5 equiv.) in pyridine (3 mL) was stirred at 40° C. for 1 hour. The reaction mixture was diluted with H₂O (5 mL) and concentrated to a turbid liquid. The pH of the turbid liquid was adjusted to pH=3 with 1 N HCl (5 mL). The residue was extracted with DCM/MEOH (10:1). The combined organic layers were filtered and concentrated to afford tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate (200 mg, crude) as a red solid.

LC-MS (ESI): m/z=542.1 [M/2+H]⁺.

### Step 7: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid

A mixture of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate (200 mg, 184.81 mol, 1 equiv.) in TFA (1 mL) and DCM (1 mL) was stirred at 25° C. for 6 hours. The combined organic layers were filtered and concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid (84.29 mg) as a white solid.

LC-MS (ESI): m/z=1026.3 [M+H]⁺

¹H NMR (400 MHz, DMSO-d₆) δ=12.96-12.75 (m, 1H), 12.62-12.26 (m, 1H), 10.88 (s, 1H), 10.04-9.54 (m, 1H), 8.06-7.99 (m, 2H), 7.79 (d, J=8.0 Hz, 1H), 7.67-7.59 (m, 2H), 7.49-7.43 (m, 3H), 7.36 (td, J=7.6, 11.6 Hz, 2H), 7.22-7.13 (m, 4H), 6.99 (d, J=8.8 Hz, 1H), 5.06-4.94 (m, 2H), 4.33 (dd, J=4.4, 10.0 Hz, 1H), 4.05 (d, J=6.0 Hz, 2H), 3.96-3.91 (m, 5H), 3.21 (d, J=4.4 Hz, 2H), 3.03 (t, J=6.0 Hz, 2H), 2.73-2.61 (m, 4H), 2.42-2.27 (m, 4H), 2.17 (dd, J=5.2, 13.2 Hz, 1H), 2.01-1.91 (m, 2H), 1.81-1.61 (m, 6H)

### Example 9: 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid] (Compound 130a)

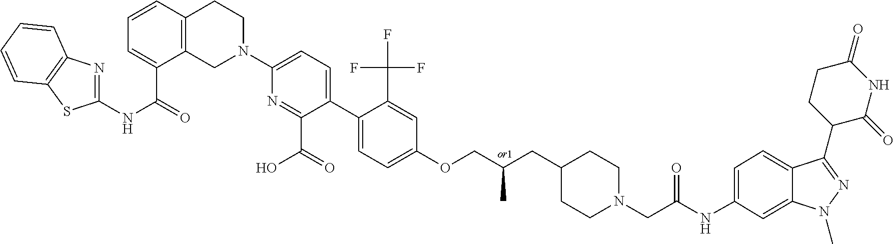 [SMILES: C[C@@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1]

### Step 1: tert-butyl 4-(3-(4-bromo-3-(trifluoromethyl)phenoxy)-2-methylpropyl)piperidine-1-carboxylate

To a solution of tert-butyl 4-(3-hydroxy-2-methylpropyl)piperidine-1-carboxylate (1.5 g, 5.83 mmol, 1.0 equiv.) in toluene (15 mL) was added 2-(tributyl-phosphanylidene)acetonitrile (2.11 g, 8.74 mmol, 1.5 equiv.) and 4-bromo-3-(trifluoromethyl)phenol (1.69 g, 6.99 mmol, 1.2 equiv.). The mixture was degassed and purged with N₂ three times. The mixture was stirred at 120° C. for 12 hours under a N₂ atmosphere. The reaction mixture was concentrated under reduced pressure to remove solvent. The crude product was purified by reverse-phase HPLC to give tert-butyl 4-(3-(4-bromo-3-(trifluoromethyl)phenoxy)-2-methylpropyl)piperidine-1-carboxylate (2.8 g) as a yellow oil.

### Step 2: tert-butyl (R)-4-(3-(4-bromo-3-(trifluoromethyl)phenoxy)-2-methylpropyl)piperidine-1-carboxylate and tert-butyl (S)-4-(3-(4-bromo-3-(trifluoromethyl)phenoxy)-2-methylpropyl)piperidine-1-carboxylate

The stereoisomers of tert-butyl 4-(3-(4-bromo-3-(trifluoromethyl)phenoxy)-2-methylpropyl)piperidine-1-carboxylate were further separated by SFC (column: Daicel Chiralpak IC (250 mm×30 mm, 10 μm); mobile phase: [CO₂-iPrOH (0.1% NH₃·H₂O)]; B % %, isocratic elution mode) to give tert-butyl (S)-4-(3-(4-bromo-3-(trifluoromethyl)phenoxy)-2-methylpropyl)piperidine-1-carboxylate (Peak 1) (950 mg) and tert-butyl (R)-4-(3-(4-bromo-3-(trifluoromethyl)phenoxy)-2-methylpropyl)piperidine-1-carboxylate (Peak 2) (950 mg) as a yellow oil.

Peak 1:

SFC: t_{R}=1.143 min

¹H NMR (400 MHz, DMSO-d₆) δ=7.75 (d, J=8.8 Hz, 1H), 7.31 (s, 1H), 7.18 (d, J=8.8 Hz, 1H), 3.97-3.86 (m, 3H), 3.85-3.80 (m, 1H), 2.67 (s, 2H), 2.04-1.94 (m, 1H), 1.62 (d, J=13.2 Hz, 2H), 1.54 (s, 1H), 1.38 (s, 9H), 1.16-1.08 (m, 1H), 1.04 (d, J=6.0 Hz, 2H), 0.97 (d, J=6.4 Hz, 3H), 0.93-0.84 (m, 1H).

Peak 2:

SFC: t_{R}=1.284 min

¹H NMR (400 MHz, DMSO-d₆) δ=7.75 (d, J=8.8 Hz, 1H), 7.31 (s, 1H), 7.18 (d, J=9.2 Hz, 1H), 3.98-3.86 (m, 3H), 3.85-3.78 (m, 1H), 2.67 (s, 2H), 2.04-1.94 (m, 1H), 1.62 (d, J=12.8 Hz, 2H), 1.54 (s, 1H), 1.38 (s, 9H), 1.12 (td, J=6.8, 13.6 Hz, 1H), 1.04 (d, J=6.0 Hz, 2H), 0.97 (d, J=6.4 Hz, 3H), 0.93-0.83 (m, 1H)

### Step 3: (R)-4-(3-(4-bromo-3-(trifluoromethyl)phenoxy)-2-methylpropyl)piperidine

To a solution of tert-butyl (R)-4-(3-(4-bromo-3-(trifluoromethyl)phenoxy)-2-methylpropyl)piperidine-1-carboxylate (950 mg, 1.98 mmol, 1.0 equiv.) in DCM (10 mL) was added HCl/dioxane (4 M, 494.42 μL, 1.0 equiv.). The mixture was stirred at 25° C. for 1 hour. The reaction mixture was filtered and concentrated under reduced pressure to give (R)-4-(3-(4-bromo-3-(trifluoromethyl)phenoxy)-2-methylpropyl)piperidine (800 mg) as a white solid, which was used in the next step without further purification.

### Step 4: ethyl (R)-2-(4-(3-(4-bromo-3-(trifluoromethyl)phenoxy)-2-methylpropyl)piperidin-1-yl)acetate

To a solution of (R)-4-(3-(4-bromo-3-(trifluoromethyl)phenoxy)-2-methylpropyl)piperidine (300 mg, 719.94 mol, 1.0 equiv., HCl) in DMF (3 mL) was added potassium carbonate (298.50 mg, 2.16 mmol, 3.0 equiv.) and ethyl 2-bromoacetate (120.23 mg, 719.94 mol, 79.68 μL, 1.0 equiv.). The mixture was stirred at 40° C. for 1 hour. The reaction mixture was concentrated under reduced pressure to remove solvent. The residue was diluted with H₂O (50 mL) and extracted with ethyl acetate (100 mL). The combined organic layers were washed with H₂O (50 mL), dried over sodium sulfate, filtered, and concentrated under reduced pressure to give ethyl (R)-2-(4-(3-(4-bromo-3-(trifluoromethyl)phenoxy)-2-methylpropyl)piperidin-1-yl)acetate (300 mg) as a colorless oil, which was used in the next step without further purification.

### Step 5: tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate

A mixture of ethyl (R)-2-(4-(3-(4-bromo-3-(trifluoromethyl)phenoxy)-2-methylpropyl)piperidin-1-yl)acetate (250 mg, 536.10 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (361.22 mg, 589.71 mol, 1.1 equiv.), KF (93.44 mg, 1.61 mmol, 37.68 μL, 3.0 equiv.), Ad₂nBup Pd G₃ (39.04 mg, 53.61 mol, 0.1 equiv.) and H₂O (0.3 mL) in dioxane (3 mL) was degassed and purged with N₂ three times. The mixture was stirred at 100° C. for 1 hour under a N₂ atmosphere. The reaction mixture was concentrated under reduced pressure to remove solvent. The residue was purified by column chromatography (SiO₂, petroleum ether/ethyl acetate=10/1 to 0/1) to give tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate (300 mg) as a yellow oil.

LC-MS (ESI): m/z=872.2 [M+H]⁺

¹H NMR (400 MHz, DMSO-d₆) δ=12.85 (s, 1H), 8.01 (d, J=7.6 Hz, 1H), 7.77 (d, J=8.0 Hz, 1H), 7.63-7.56 (m, 1H), 7.44 (q, J=8.8 Hz, 3H), 7.39-7.31 (m, 2H), 7.26-7.16 (m, 2H), 7.15-7.09 (m, 1H), 6.95 (d, J=8.8 Hz, 1H), 5.07-4.92 (m, 2H), 4.06 (q, J=6.8 Hz, 2H), 3.96-3.78 (m, 4H), 3.15 (s, 2H), 3.07-2.98 (m, 2H), 2.78 (d, J=10.4 Hz, 2H), 2.11 (q, J=10.4 Hz, 2H), 1.99 (s, 1H), 1.60 (d, J=12.0 Hz, 2H), 1.33 (s, 2H), 1.20-1.13 (m, 4H), 1.11 (d, J=8.4 Hz, 2H), 1.02 (s, 9H), 0.96 (d, J=6.4 Hz, 3H).

### Step 6: (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid

To a solution of tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate (250 mg, 286.70 mol, 1.0 equiv.) in THE (3 mL) was added LiOH·H₂O (36.09 mg, 860.09 mol, 3.0 equiv.) and H₂O (0.6 mL). The mixture was stirred at 40° C. for 1 hour. The mixture was concentrated under reduced pressure to remove solvent. The reaction mixture was acidified to pH=5-6 with 1 M HCl, filtered, and concentrated under reduced pressure to give (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid (230 mg) as a yellow solid, which was used in the next step without further purification.

### Step 7: tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate

To a solution of (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid (180 mg, 213.28 mol, 1.0 equiv.) in pyridine (1 mL) was added EDCI (61.33 mg, 319.92 mol, 1.5 equiv.) and 3-(6-amino-1-methyl-indazol-3-yl)piperidine-2,6-dione (66.10 mg, 255.94 mol, 1.2 equiv.). The mixture was stirred at 40° C. for 1 hour. The reaction mixture was concentrated under reduced pressure to remove solvent. The residue was diluted with H₂O (100 mL), extracted with DCM (300 mL), washed with brine (50 mL), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate (230 mg) as a yellow oil, which was used in the next step without further purification.

### Step 8: 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid]

To a solution of tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate (230 mg, 212.14 mol, 1.0 equiv.) in TFA (2 mL) was added DCM (1 mL). The mixture was stirred at 40° C. for 1 hour. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid] (207.14 mg) as a white solid.

LC-MS (ESI): m/z=1028.3 [M+H]⁺

¹H NMR (400 MHz, DMSO-d₆) δ=12.98-12.74 (m, 1H), 10.88 (s, 1H), 10.15-9.75 (m, 1H), 8.06-8.01 (m, 2H), 7.79 (d, J=8.0 Hz, 1H), 7.66-7.61 (m, 2H), 7.49-7.43 (m, 3H), 7.36 (td, J=7.6, 12.0 Hz, 2H), 7.21-7.14 (m, 4H), 6.99 (d, J=8.8 Hz, 1H), 5.07-4.93 (m, 2H), 4.33 (dd, J=5.2, 9.6 Hz, 1H), 3.96-3.91 (m, 6H), 3.87-3.82 (m, 1H), 3.03 (t, J=5.6 Hz, 4H), 2.68-2.59 (m, 2H), 2.39-2.31 (m, 2H), 2.26-2.12 (m, 2H), 2.11-1.92 (m, 2H), 1.76-1.66 (m, 2H), 1.47-1.13 (m, 6H), 1.00 (d, J=6.4 Hz, 3H).

### Example 10: 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid] (Compound 130b)

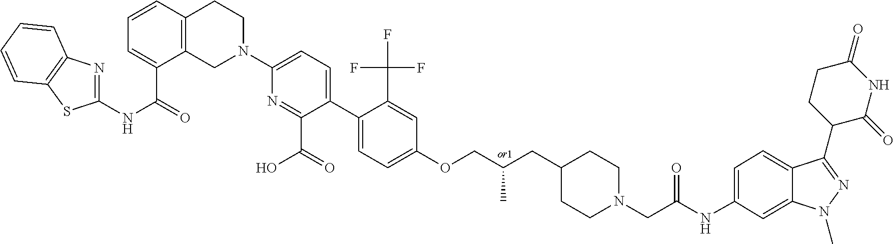 [SMILES: C[C@H](COc1ccc(-c2ccc(N3CCc4cccc(C(=O)Nc5nc6ccccc6s5)c4C3)nc2C(=O)O)c(C(F)(F)F)c1)CC1CCN(CC(=O)Nc2ccc3c(C4CCC(=O)NC4=O)nn(C)c3c2)CC1]

### Step 1: (S)-4-(3-(4-bromo-3-(trifluoromethyl)phenoxy)-2-methylpropyl)piperidine

To a solution of tert-butyl (S)-4-(3-(4-bromo-3-(trifluoromethyl)phenoxy)-2-methylpropyl)piperidine-1-carboxylate (950 mg, 1.98 mmol, 1.0 equiv.) in EtOAc (2 mL) was added HCl/dioxane (10 mL). The mixture was stirred at 25° C. for 1 hour. The reaction mixture was concentrated under reduced pressure to give (S)-4-(3-(4-bromo-3-(trifluoromethyl)phenoxy)-2-methylpropyl)piperidine (800 mg) as a pink solid, which was used for next step without purification.

### Step 2: ethyl (S)-2-(4-(3-(4-bromo-3-(trifluoromethyl)phenoxy)-2-methylpropyl)piperidin-1-yl)acetate

To a solution of (S)-4-(3-(4-bromo-3-(trifluoromethyl)phenoxy)-2-methylpropyl)piperidine (250 mg, 599.95 mol, 1.0 equiv., HCl) and potassium carbonate (248.75 mg, 1.80 mmol, 3.0 equiv.) in DMF (5 mL) was added ethyl 2-bromoacetate (100.19 mg, 599.95 mol, 66.40 μL, 1.0 equiv.). The mixture was stirred at 40° C. for 1 hour. Ethyl acetate (100 mL) and H₂O (50 mL) were added and the layers were separated. The aqueous layer was extracted with ethyl acetate (60 mL), dried over anhydrous sodium sulfate, filtered, and concentrated under vacuum to give ethyl (S)-2-(4-(3-(4-bromo-3-(trifluoromethyl)phenoxy)-2-methylpropyl)piperidin-1-yl)acetate (260 mg) as a yellow oil, which was used for next step without purification.

LC-MS (ESI): m/z=466.1 [M+H]⁺

¹H NMR (400 MHz, DMSO-d6) δ=7.75 (d, J=8.8 Hz, 1H), 7.31 (s, 1H), 7.18 (d, J=8.4 Hz, 1H), 4.10-4.04 (m, 2H), 3.92-3.88 (m, 1H), 3.83-3.79 (m, 1H), 3.15 (s, 2H), 2.80-2.73 (m, 3H), 2.15-2.08 (m, 2H), 1.97 (d, J=6.4 Hz, 1H), 1.60 (d, J=12.4 Hz, 2H), 1.42-1.27 (m, 2H), 1.18 (t, J=7.2 Hz, 3H), 1.14-1.02 (m, 2H), 0.95 (d, J=6.4 Hz, 3H)

### Step 3: tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate

A mixture of ethyl (S)-2-(4-(3-(4-bromo-3-(trifluoromethyl)phenoxy)-2-methylpropyl)piperidin-1-yl)acetate (260 mg, 557.54 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (409.82 mg, 669.05 mol, 1.2 equiv.), H₂O (0.8 mL), KF (97.17 mg, 1.67 mmol, 39.18 μL, 3.0 equiv.), and Ad₂nBuP Pd G₃ (40.60 mg, 55.75 mol, 0.1 equiv.) in dioxane (8 mL) was degassed and purged with N₂ three times. The mixture was stirred at 100° C. for 1 hour under a N₂ atmosphere. The reaction mixture was concentrated under reduced pressure to give a residue, which was purified by column chromatography (SiO₂, petroleum ether/ethyl acetate=3/1 to 1/1) to give tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate (270 mg) as a yellow solid.

### Step 4: (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid

To a solution of tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-(trifluoromethyl)phenyl)picolinate (270 mg, 309.63 mol, 1.0 equiv.) in THE (1 mL) was added LiOH·H₂O (38.98 mg, 928.90 mol, 3.0 equiv.) and H₂O (0.2 mL). The mixture was stirred at 40° C. for 1 hour. The reaction mixture was concentrated under reduced pressure to remove the solvent. The reaction mixture was added to water (3 mL). The pH was adjusted to pH=6 by progressively adding diluted 1M HCl and filtered to give (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid (240 mg) as a yellow solid, which was used for next step without purification.

### Step 5: tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate

To a solution of (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-2-methylpropyl)piperidin-1-yl)acetic acid (240 mg, 284.38 mol, 1.0 equiv.) in pyridine (2 mL) was added EDCI (81.77 mg, 426.57 mol, 1.5 equiv.) and 3-(6-amino-1-methyl-indazol-3-yl)piperidine-2,6-dione (100.58 mg, 341.25 mol, 1.2 equiv., HCl). The mixture was stirred at 40° C. for 1 hour. DCM (100 mL) and H₂O (50 mL) were added, and layers were separated. The aqueous layer was extracted with DCM (60 mL), and the organic layer was dried over anhydrous sodium sulfate, filtered, and concentrated under vacuum to give tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate (280 mg, crude) as a yellow solid, which was used for next step without purification.

### Step 6: 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid]

To a solution of tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinate (280 mg, 258.25 mol, 1.0 equiv.) in DCM (2 mL) was added TFA (4 mL). The mixture was stirred at 40° C. for 1 hour. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-(trifluoromethyl)phenyl)picolinic acid] (84.41 mg) as a yellow solid.

LC-MS (ESI): m/z=1028.3 [M+H]⁺

¹H NMR (400 MHz DMSO-d₆) δ=10.88 (s, 1H), 9.84 (s, 1H), 8.05-8.01 (m, 2H), 7.79 (d, J=8.0 Hz, 1H), 7.63 (d, J=8.8 Hz, 2H), 7.47-7.43 (m, 3H), 7.40-7.34 (m, 2H), 7.23-7.15 (m, 4H), 6.99 (d, J=8.8 Hz, 1H), 5.11-4.90 (m, 2H), 4.40-4.25 (m, 1H), 3.99-3.88 (m, 6H), 3.87-3.81 (m, 1H), 3.14 (s, 2H), 3.03 (t, J=5.6 Hz, 2H), 2.89 (d, J=10.8 Hz, 2H), 2.70-2.59 (m, 2H), 2.38-2.30 (m, 1H), 2.23-2.12 (m, 3H), 2.08-1.98 (m, 1H), 1.67 (d, J=10.2 Hz, 2H), 1.47-1.38 (m, 2H), 1.34-1.14 (m, 3H), 1.00 (d, J=6.4 Hz, 3H)

### Example 11: 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid] (Compound 138a)

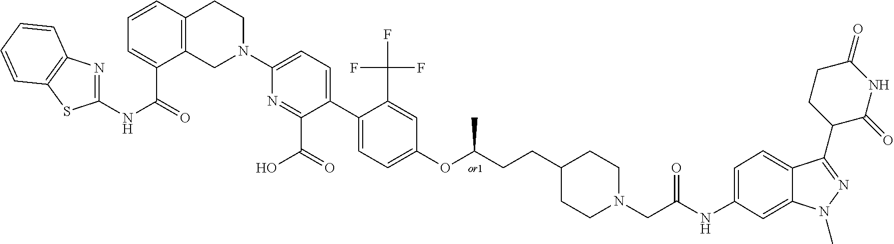 [SMILES: C[C@@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1]

### Step 1: tert-butyl 4-(3-(methoxy(methyl)amino)-3-oxopropyl)piperidine-1-carboxylate

To a solution of 3-(1-(tert-butoxycarbonyl)piperidin-4-yl)propanoic acid (10 g, 38.86 mmol, 1 equiv.), N,O-dimethylhydroxylamine hydrochloride (11.37 g, 116.58 mmol, 3 equiv.) in DCM (2 mL) was added HA TU (22.16 g, 58.29 mmol, 1.5 equiv.) and TEA (19.66 g, 194.31 mmol, 27.04 mL, 5 equiv.). The mixture was stirred at 25° C. for 1 hour. The reaction mixture was concentrated under reduced pressure to give a residue, which was purified by flash silica gel chromatography (0~700 ethyl acetate/petroleum ether) to give tert-butyl 4-(3-(methoxy(methyl)amino)-3-oxopropyl)piperidine-1-carboxylate (7.5 g) as a yellow oil.

¹H NMR (400 MHz, DMSO-d₆) δ=3.91 (d, J=11.2 Hz, 2H), 3.65 (s, 3H), 3.07 (s, 3H), 2.67 (d, J=16.4 Hz, 2H), 2.38 (t, J=6.4 Hz, 2H), 1.62 (d, J=12.4 Hz, 2H), 1.43-1.33 (m, 12H), 1.04-0.86 (in, 2H)

### Step 2: tert-butyl 4-(3-oxobutyl)piperidine-1-carboxylate

A mixture of tert-butyl 4-(3-(methoxy(methyl)amino)-3-oxopropyl)piperidine-1-carboxylate (7.5 g, 24.97 mmol, 1 equiv.) in THF (85 mL) was degassed and purged with N₂ three times. To this mixture was slowly added MeMgBr (2.5 M, 19.97 mL, 2 equiv.). The resulting mixture was stirred at −70° C. for 0.5 hours under a N₂ atmosphere. The mixture was slowly warm to 25° C. and stirred for 5.5 hours under a N₂ atmosphere. The reaction mixture was quenched by the addition of saturated ammonium chloride (100 mL) and extracted with DCM (200 mL). The combined organic layers were washed with brine (50 mL), dried over sodium sulfate, filtered, and concentrated under reduced pressure to give tert-butyl 4-(3-oxobutyl)piperidine-1-carboxylate (7.2 g, crude) as a yellow oil.

¹H NMR (400 MHz, DMSO-d₆) δ=3.90 (d, J=12.0 Hz, 2H), 2.71-2.55 (m, 2H), 2.44 (t, J=7.6 Hz, 2H), 2.07 (s, 3H), 1.59 (d, J=12.4 Hz, 2H), 1.38 (s, 9H), 1.36-1.27 (m, 2H), 0.97-0.86 (m, 2H)

### Step 3: tert-butyl 4-(3-hydroxybutyl)piperidine-1-carboxylate

To a solution of tert-butyl 4-(3-oxobutyl)piperidine-1-carboxylate (7.2 g, 28.20 mmol, 1 equiv.) in EtOH (72 mL) was added NaBH₄ (1.28 g, 33.84 mmol, 1.2 equiv.). The mixture was stirred at 0° C. for 2 hours. The reaction mixture was quenched by the addition of saturated ammonium chloride (100 mL) and extracted with DCM (200 mL). The combined organic layers were washed with brine (50 mL), dried over sodium sulfate, filtered, and concentrated under reduced pressure to give a residue, which was purified by flash silica gel chromatography (0~20% ethyl acetate/petroleum ether) to give tert-butyl 4-(3-hydroxybutyl)piperidine-1-carboxylate (8 g) as a yellow oil.

¹H NMR (400 MHz, DMSO-d₆) δ=4.31 (d, J=4.0 Hz, 1H), 3.90 (d, J=11.2 Hz, 2H), 3.59-3.48 (m, 1H), 3.32 (s, 1H), 2.65 (s, 2H), 1.60 (d, J=12.8 Hz, 2H), 1.38 (s, 9H), 1.29 (s, 3H), 1.21-1.09 (m, 1H), 1.02 (d, J=6.0 Hz, 3H), 0.92 (q, J=11.6 Hz, 2H)

### Step 4: tert-butyl 4-(3-(4-bromo-3-(trifluoromethyl)phenoxy)butyl)piperidine-1-carboxylate

To a solution of tert-butyl 4-(3-hydroxybutyl)piperidine-1-carboxylate (4.8 g, 18.65 mmol, 1 equiv.) and 4-bromo-3-(trifluoromethyl)phenol (4.94 g, 20.52 mmol, 1.1 equiv.) in toluene (70 mL) was added 2-(tributyl-phosphanylidene)acetonitrile (6.75 g, 27.98 mmol, 1.5 equiv.). The mixture was stirred at 120° C. for 12 hours. The reaction mixture was concentrated under reduced pressure to give a residue, which was purified by prep-HPLC to give tert-butyl 4-(3-(4-bromo-3-(trifluoromethyl)phenoxy)butyl)piperidine-1-carboxylate (5.8 g) as a black oil.

### Step 5: tert-butyl (S)-4-(3-(4-bromo-3-(trifluoromethyl)phenoxy)butyl)piperidine-1-carboxylate and tert-butyl (R)-4-(3-(4-bromo-3-(trifluoromethyl)phenoxy)butyl)piperidine-1-carboxylate

Enantiomers of tert-butyl 4-(3-(4-bromo-3-(trifluoromethyl)phenoxy)butyl)piperidine-1-carboxylate were further separated by SFC (column: Daicel Chiralcel OJ (250 mm×30 mm, 10 μm); mobile phase: [CO₂-iPrOH]; B %: 7%, isocratic elution mode) to give tert-butyl (S)-4-(3-(4-bromo-3-(trifluoromethyl)phenoxy)butyl)piperidine-1-carboxylate (Peak 1) (630 mg) as a white solid and tert-butyl (R)-4-(3-(4-bromo-3-(trifluoromethyl)phenoxy)butyl)piperidine-1-carboxylate (Peak 2) (630 mg) as a white solid.

Peak 1:

SFC: t_{R}=1.022 min

¹H NMR (400 MHz, DMSO-d₆) δ=7.74 (d, J=8.6 Hz, 1H), 7.27 (s, 1H), 7.21-7.13 (m, 1H), 4.60-4.48 (m, 1H), 3.96-3.85 (m, 2H), 2.71-2.57 (m, 2H), 1.61 (d, J=11.2 Hz, 4H), 1.38 (s, 10H), 1.34-1.28 (m, 1H), 1.23 (d, J=6.4 Hz, 4H), 1.00-0.86 (m, 2H)

Peak 2:

SFC: t_{R}=1.123 min

¹H NMR (400 MHz, DMSO-d₆) δ=7.74 (d, J=8.8 Hz, 1H), 7.27 (s, 1H), 7.18 (d, J=8.4 Hz, 1H), 4.59-4.48 (m, 1H), 3.90 (d, J=12.0 Hz, 2H), 2.64 (d, J=17.2 Hz, 2H), 1.67-1.51 (m, 4H), 1.38 (s, 10H), 1.33-1.27 (m, 1H), 1.23 (d, J=6.0 Hz, 4H), 1.01-0.85 (m, 2H)

### Step 6: (S)-4-(3-(4-bromo-3-(trifluoromethyl)phenoxy)butyl)piperidine

A solution of tert-butyl (S)-4-(3-(4-bromo-3-(trifluoromethyl)phenoxy)butyl)piperidine-1-carboxylate (630 mg, 1.31 mmol, 1 equiv.) in HCl/dioxane (6.3 mL, 2M) was stirred at 25° C. for 12 hours. The reaction mixture was concentrated under reduced pressure to give (S)-4-(3-(4-bromo-3-(trifluoromethyl)phenoxy)butyl)piperidine (520 mg, crude) as a colorless oil.

### Step 7: ethyl (S)-2-(4-(3-(4-bromo-3-(trifluoromethyl)phenoxy)butyl)piperidin-1-yl)acetate

To a solution of (S)-4-(3-(4-bromo-3-(trifluoromethyl)phenoxy)butyl)piperidine (500 mg, 1.31 mmol, 1 equiv.) in acetone (0.5 mL) was added TEA (399.18 mg, 3.94 mmol, 549.07 L, 3 equiv.) and ethyl 2-bromoacetate (219.60 mg, 1.31 mmol, 145.53 μL, 1 equiv.). The mixture was stirred at 0° C. for 0.1 hours. The reaction mixture was concentrated under reduced pressure to give a residue, which was purified by flash silica gel chromatography (0-100% ethyl acetate/petroleum ether) to give ethyl (S)-2-(4-(3-(4-bromo-3-(trifluoromethyl)phenoxy)butyl)piperidin-1-yl)acetate (500 mg) as a colorless.

### Step 8: tert-butyl (S)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate

To a solution of ethyl (S)-2-(4-(3-(4-bromo-3-(trifluoromethyl)phenoxy)butyl)piperidin-1-yl)acetate (200 mg, 428.88 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (288.98 mg, 471.77 mol, 1.1 equiv.) in dioxane (2 mL) and H₂O (0.2 mL) was added Ad₂nBup Pd G₃ (31.23 mg, 42.89 mol, 0.1 equiv.) and KF (74.75 mg, 1.29 mmol, 30.14 μL, 3 equiv.). The mixture was stirred at 100° C. for 3 hours. The reaction mixture was concentrated under reduced pressure to give a residue, which was purified by flash silica gel chromatography (0~10% DCM/MeOH) to give tert-butyl (S)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate (400 mg) as a yellow solid.

### Step 9: (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid

To a solution of tert-butyl (S)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate (400.00 mg, 458.71 mol, 1 equiv.) in THF (4 mL) and H₂O (0.4 mL) was added LiOH·H₂O (57.75 mg, 1.38 mmol, 3 equiv.). The mixture was stirred at 25° C. for 12 hours. The reaction mixture was concentrated under reduced pressure to give a residue, which was treated with water (6 mL). The pH was adjusted to pH 4.5 by progressively adding diluted HCl. The mixture was filtered to give (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid (400 mg) as a white solid.

### Step 10: tert-butyl 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate

To a solution of (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid (400 mg, 473.96 mol, 1 equiv.), 3-(6-amino-1-methyl-indazol-3-yl)piperidine-2,6-dione (167.63 mg, 568.75 mol, 1.2 equiv. HCl) in pyridine (4 mL) was added EDCI (136.29 mg, 710.94 mol, 1.5 equiv.). The mixture was stirred at 25° C. for 2 hours. The reaction mixture was partitioned between DCM (15 mL) and H₂O (5 mL). The organic phase was separated, dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate (500 mg) as a white solid.

### Step 11: 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid]

To a solution of tert-butyl 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate (500 mg, 461.16 mol, 1 equiv.) in TFA (2.5 mL) was added DCM (5 mL). The mixture was stirred at 25° C. for 12 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rei-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid] (104.52 mg) as a pink solid.

LC-MS (ESI): m/z=1028.3 [M+H]⁺

¹H NMR (400 MHz, DMSO-d₆) δ=13.16-12.43 (m, 1H), 10.87 (s, 1H), 10.16-9.75 (m, 1H), 8.13 (s, 1H), 8.09-7.97 (m, 2H), 7.78 (d, J=8.0 Hz, 1H), 7.68-7.57 (m, 2H), 7.50-7.41 (m, 3H), 7.41-7.30 (m, 2H), 7.23-7.11 (m, 4H), 6.99 (d, J=8.8 Hz, 1H), 5.11-4.86 (m, 2H), 4.60-4.49 (m, 1H), 4.32 (dd, J=4.8, 9.6 Hz, 1H), 4.00-3.88 (m, 5H), 3.06-2.92 (m, 4H), 2.75-2.52 (m, 4H), 2.40-2.12 (m, 4H), 1.77-1.56 (m, 4H), 1.46-1.21 (m, 8H)

### Example 12: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylic acid (Compound 135)

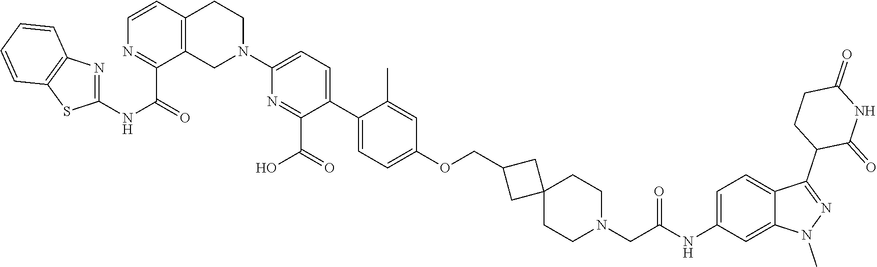 [SMILES: Cc1cc(OCC2CC3(CCN(CC(=O)Nc4ccc5c(C6CCC(=O)NC6=O)nn(C)c5c4)CC3)C2)ccc1-c1ccc(N2CCc3ccnc(C(=O)Nc4nc5ccccc5s4)c3C2)nc1C(=O)O]

### Step 1: tert-butyl 2-[(4-bromo-3-methyl-phenoxy)methyl]-7-azaspiro[3.5]nonane-7-carboxylate

A mixture of tert-butyl 2-(hydroxymethyl)-7-azaspiro[3.5]nonane-7-carboxylate (1.0 g, 3.92 mmol, 1.0 equiv.), 4-bromo-3-methyl-phenol (878.94 mg, 4.70 mmol, 1.2 equiv.), 2-(tributyl-phosphanylidene)acetonitrile (1.42 g, 5.87 mmol, 1.5 equiv.) in toluene (100 mL) was degassed and purged with N₂ three times. The mixture was stirred at 120° C. for 16 hours under a N₂ atmosphere. The reaction mixture was concentrated under reduced pressure to give a residue, which was purified by flash silica gel chromatography (0-5% ethyl acetate/petroleum ether) to give tert-butyl 2-[(4-bromo-3-methyl-phenoxy)methyl]-7-azaspiro[3.5]nonane-7-carboxylate (2.0 g) as a yellow oil.

LC-MS (ESI): m/z=368.1 [M+H−56]⁺

### Step 2: 2-[(4-bromo-3-methyl-phenoxy)methyl]-7-azaspiro[3.5]nonane

A mixture of tert-butyl 2-[(4-bromo-3-methyl-phenoxy)methyl]-7-azaspiro[3.5]nonane-7-carboxylate (2.0 g, 4.71 mmol, 1.0 equiv.) in HCl/dioxane (100 mL) was stirred at 25° C. for 1 hour. The reaction mixture was concentrated under reduced pressure to give a residue, which was washed with petroleum ether:ethyl acetate=3/1 (50 mL) to give 2-[(4-bromo-3-methyl-phenoxy)methyl]-7-azaspiro[3.5]nonane (1.3 g) as a white solid.

### Step 3: ethyl 2-[2-[(4-bromo-3-methyl-phenoxy)methyl]-7-azaspiro[3.5]nonan-7-yl]acetate

A mixture of ethyl 2-bromoacetate (669.54 mg, 4.01 mmol, 443.70 μL, 1.0 equiv.), 2-[(4-bromo-3-methyl-phenoxy)methyl]-7-azaspiro[3.5]nonane (1.3 g, 4.01 mmol, 1.0 equiv.), potassium carbonate (1.66 g, 12.03 mmol, 3.0 equiv.) in DMF (10 mL) was degassed and purged with N₂ three times. The mixture was stirred at 40° C. for 1 hour under a N₂ atmosphere. The reaction mixture was diluted with H₂O (100 mL) and extracted with EtOAc (100 mL). The combined organic layers were washed with brine (600 mL), dried over sodium sulfate, filtered, and concentrated under reduced pressure to give ethyl 2-[2-[(4-bromo-3-methyl-phenoxy)methyl]-7-azaspiro[3.5]nonan-7-yl]acetate (5 g) as a yellow oil.

¹H NMR (400 MHz, DMSO-d₆) δ=7.41 (d, J=8.8 Hz, 1H), 6.94 (s, 1H), 6.70 (d, J=8.8 Hz, 1H), 4.06 (q, J=7.2 Hz, 2H), 3.90 (d, J=6.4 Hz, 2H), 3.14 (s, 2H), 2.62 (td, J=7.9, 15.2 Hz, 1H), 2.42 (s, 2H), 2.34 (s, 2H), 2.29 (s, 3H), 1.86 (t, J=10.0 Hz, 2H), 1.62-1.44 (m, 6H), 1.18 (t, J=7.2 Hz, 3H).

### Step 4: 2-[2-[(4-bromo-3-methyl-phenoxy)methyl]-7-azaspiro[3.5]nonan-7-yl]acetic acid

A mixture of ethyl 2-[2-[(4-bromo-3-methyl-phenoxy)methyl]-7-azaspiro[3.5]nonan-7-yl]acetate (1.0 g, 2.44 mmol, 1.0 equiv.), LiOH·H₂O (409.06 mg, 9.75 mmol, 4.0 equiv.) in THE (8 mL) and H₂O (2 mL) was degassed and purged with N₂ three times. The mixture was stirred at 40° C. for 2 hours under a N₂ atmosphere. The reaction mixture was concentrated under reduced pressure to remove THF. The residue pH was adjusted to pH=3-4 with HCl. The reaction mixture was quenched by the addition of H₂O (100 mL) and extracted with EtOAc (500 mL). The combined organic layers were washed with brine (100 mL), dried over sodium sulfate, filtered, and concentrated under reduced pressure to give 2-[2-[(4-bromo-3-methyl-phenoxy)methyl]-7-azaspiro[3.5]nonan-7-yl]acetic acid (1.0 g) as a brown oil.

### Step 5: 2-[2-[(4-bromo-3-methyl-phenoxy)methyl]-7-azaspiro[3.5]nonan-7-yl]-N-[3-(2,6-dioxo-3-piperidyl)-1-methyl-indazol-6-yl]acetamide

A mixture of 3-(6-amino-1-methyl-indazol-3-yl)piperidine-2,6-dione (810.72 mg, 2.75 mmol, 1.05 equiv., HCl), 2-[2-[(4-bromo-3-methyl-phenoxy)methyl]-7-azaspiro[3.5]nonan-7-yl]acetic acid (1.0 g, 2.62 mmol, 1.0 equiv.), and EDCI (752.18 mg, 3.92 mmol, 1.5 equiv.) in pyridine (10 mL) was degassed and purged with N₂ three times. The mixture was stirred at 60° C. for 12 hours under a N₂ atmosphere. The reaction mixture was quenched by the addition of H₂O (100 mL) and extracted with DCM (500 mL). The combined organic layers were washed with brine (100 mL), dried over sodium sulfate, filtered, and concentrated under reduced pressure to give 2-[2-[(4-bromo-3-methyl-phenoxy)methyl]-7-azaspiro[3.5]nonan-7-yl]-N-[3-(2,6-dioxo-3-piperidyl)-1-methyl-indazol-6-yl]acetamide (1.5 g) as a brown solid.

### Step 6: N-[3-(2,6-dioxo-3-piperidyl)-1-methyl-indazol-6-yl]-2-[2-[[3-methyl-4-(4,4,5,5-tetramethyl-1,3,2-dioxaborolan-2-yl)phenoxy]methyl]-7-azaspiro[3.5]nonan-7-yl]acetamide

A mixture of 2-[2-[(4-bromo-3-methyl-phenoxy)methyl]-7-azaspiro[3.5]nonan-7-yl]-N-[3-(2,6-dioxo-3-piperidyl)-1-methyl-indazol-6-yl]acetamide (700 mg, 1.12 mmol, 1.0 equiv.), bis(pinacolato)diboron (856.59 mg, 3.37 mmol, 3.0 equiv.), KOAc (331.05 mg, 3.37 mmol, 3.0 equiv.), and Pd(dppf)Cl₂ (164.55 mg, 224.88 mol, 0.2 equiv.) in dioxane (20 mL) was degassed and purged with N₂ three times. The mixture was stirred at 90° C. for 12 hours under a N₂ atmosphere. The reaction mixture was concentrated under reduced pressure to give a residue, which was purified by flash silica gel chromatography (0~100% ethyl acetate/petroleum ether) to give N-[3-(2,6-dioxo-3-piperidyl)-1-methyl-indazol-6-yl]-2-[2-[[3-methyl-4-(4,4,5,5-tetramethyl-1,3,2-dioxaborolan-2-yl)phenoxy]methyl]-7-azaspiro[3.5]nonan-7-yl]acetamide (520.0 mg) as a brown solid.

LC-MS (ESI): m/z=580.5 [M/2+H]⁺

### Step 7: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylate

A mixture of N-[3-(2,6-dioxo-3-piperidyl)-1-methyl-indazol-6-yl]-2-[2-[[3-methyl-4-(4,4,5,5-tetramethyl-1,3,2-dioxaborolan-2-yl)phenoxy]methyl]-7-azaspiro[3.5]nonan-7-yl]acetamide (220.0 mg, 328.55 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (274.69 mg, 394.26 mol, 1.2 equiv.), KF (57.26 mg, 985.64 mol, 23.09 μL, 3.0 equiv.), and Ad₂nBuP Pd G₃ (239.27 mg, 328.55 mol, 1.0 equiv.) in dioxane (10 mL) and H₂O (1 mL) was degassed and purged with N₂ three times. The mixture was stirred at 120° C. for 2 hours under a N₂ atmosphere in microwave reactor. The reaction mixture was concentrated under reduced pressure to give a residue, which was purified by flash silica gel chromatography (0~10% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylate (140.0 mg) as a yellow solid.

¹H NMR (400 MHz, DMSO-d₆) δ=10.88 (s, 1H), 9.82 (s, 1H), 8.04 (s, 1H), 7.63 (d, J=8.4 Hz, 1H), 7.55 (d, J=8.0 Hz, 1H), 7.22 (d, J=8.8 Hz, 1H), 6.75-6.67 (m, 2H), 4.32 (dd, J=4.8, 9.6 Hz, 1H), 3.97-3.88 (m, 5H), 3.10 (s, 2H), 2.74-2.58 (m, 4H), 2.44-2.27 (m, 7H), 2.17 (dd, J=5.6, 12.8 Hz, 1H), 1.95-1.88 (m, 2H), 1.68 (s, 2H), 1.64-1.54 (m, 4H), 1.27 (s, 12H).

### Step 8: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylic acid

A mixture of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylate (140.0 mg, 120.74 mol, 1.0 equiv.) in TFA (6 mL) and DCM (3 mL) was stirred at 25° C. for 2 hours. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-methyl-phenyl]pyridine-2-carboxylic acid (46.17 mg) as a yellow solid.

LC-MS (ESI): m/z=973.3 [M+H]⁺

¹H NMR (400 MHz, DMSO-d₆) δ=10.88 (s, 1H), 9.83 (s, 1H), 8.56 (d, J=4.4 Hz, 1H), 8.08-8.02 (m, 2H), 7.81 (d, J=8.4 Hz, 1H), 7.66-7.55 (m, 2H), 7.52-7.44 (m, 2H), 7.40-7.33 (m, 1H), 7.23 (d, J=8.4 Hz, 1H), 7.08-6.98 (m, 1H), 6.94 (d, J=8.4 Hz, 1H), 6.80 (s, 1H), 6.71 (d, J=7.6 Hz, 1H), 5.24 (s, 2H), 4.32 (dd, J=4.8, 9.6 Hz, 1H), 3.99-3.91 (m, 7H), 3.11 (s, 2H), 3.08 (d, J=4.4 Hz, 2H), 2.70-2.60 (m, 4H), 2.43-2.32 (m, 4H), 2.22-2.13 (m, 1H), 2.04 (s, 3H), 1.93 (t, J=10.0 Hz, 2H), 1.70 (d, J=4.0 Hz, 2H), 1.65-1.57 (m, 4H).

### Example 13: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid (Compound 141)

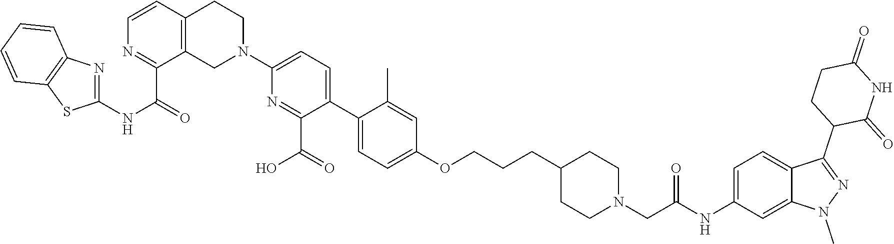 [SMILES: Cc1cc(OCCCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O]

### Step 1: tert-butyl 4-[3-(4-bromo-3-methyl-phenoxy)propyl]piperidine-1-carboxylate

To a solution of tert-butyl 4-(3-bromopropyl)piperidine-1-carboxylate (2 g, 6.53 mmol, 1 equiv.) in MeCN (20 mL) was added potassium carbonate (2.71 g, 19.59 mmol, 3 equiv.) and 4-bromo-3-methyl-phenol (1.47 g, 7.84 mmol, 1.2 equiv.). The mixture was stirred at 60° C. for 16 hours. The reaction mixture was concentrated under reduced pressure to afford tert-butyl 4-[3-(4-bromo-3-methyl-phenoxy)propyl]piperidine-1-carboxylate (3 g, crude) as a yellow oil.

LC-MS (ESI): m/z=356.0 [M−56+H]⁺.

### Step 2: 4-[3-(4-bromo-3-methyl-phenoxy)propyl]piperidine

To a solution of tert-butyl 4-[3-(4-bromo-3-methyl-phenoxy)propyl]piperidine-1-carboxylate (3 g, 7.28 mmol, 1 equiv.) in DCM (15 mL) was added HCl/dioxane (15 mL). The mixture was stirred at 25° C. for 1 hour. The reaction mixture was concentrated under reduced pressure to remove solvent. The residue was diluted with petroleum ether:EtOAc=10:1. The reaction mixture was concentrated under reduced pressure to afford 4-[3-(4-bromo-3-methyl-phenoxy)propyl]piperidine (2.29 g, HCl) as a white solid.

### Step 3: ethyl 2-[4-[3-(4-bromo-3-methyl-phenoxy)propyl]-1-piperidyl]acetate

To a solution of 4-[3-(4-bromo-3-methyl-phenoxy)propyl]piperidine (2.29 g, 6.11 mmol, 1 equiv., HCl) in DMF (40 mL) was added potassium carbonate (2.53 g, 18.32 mmol, 3 equiv.) and ethyl 2-bromoacetate (1.02 g, 6.11 mmol, 675.91 μL, 1 equiv.). The mixture was stirred at 40° C. for 1 hour. The reaction mixture was diluted with water (20 mL) and extracted with EtOAc (60 mL). The combined organic layers were dried over sodium sulfate, filtered, and concentrated under reduced pressure to give a residue, which was purified by flash silica gel chromatography (18% ethyl acetate/petroleum ether) to afford ethyl 2-[4-[3-(4-bromo-3-methyl-phenoxy)propyl]-1-piperidyl]acetate (14 g) as a white oil.

LC-MS (ESI): m/z=398.1 [M+H]⁺ 1H NMR (400 MHz, DMSO-d₆) δ=7.42 (d, J=8.8 Hz, 1H), 6.94 (d, J=2.8 Hz, 1H), 6.71 (dd, J=3.2, 8.8 Hz, 1H), 4.07 (q, J=7.2 Hz, 2H), 3.92 (t, J=6.4 Hz, 2H), 3.15 (s, 2H), 2.79 (d, J=11.6 Hz, 2H), 2.29 (s, 3H), 2.11 (t, J=10.4 Hz, 2H), 1.72-1.57 (m, 4H), 1.36-1.27 (m, 2H), 1.23-1.08 (m, 6H).

### Step 4: 2-[4-[3-(4-bromo-3-methyl-phenoxy)propyl]-1-piperidyl]acetic acid

To a solution of ethyl 2-[4-[3-(4-bromo-3-methyl-phenoxy)propyl]-1-piperidyl]acetate (2.14 g, 5.37 mmol, 1 equiv.) in THF (20 mL) and H₂O (6.6 mL) was added LiOH·H₂O (676.28 mg, 16.12 mmol, 3 equiv.). The mixture was stirred at 25° C. for 16 hours. The pH of the mixture was adjusted to pH=5-6 by HCl (1 M). The mixture was filtered and concentrated to afford 2-[4-[3-(4-bromo-3-methyl-phenoxy)propyl]-1-piperidyl]acetic acid (19 g) as a white solid.

### Step 5: 2-[4-[3-(4-bromo-3-methyl-phenoxy)propyl]-1-piperidyl]-N-[3-(2,6-dioxo-3-piperidyl)-1-methyl-indazol-6-yl]acetamide

To a solution of 2-[4-[3-(4-bromo-3-methyl-phenoxy)propyl]-1-piperidyl]acetic acid (2.19 g, 5.38 mmol, 1 equiv., HCl) and 3-(6-amino-1-methyl-indazol-3-yl)piperidine-2,6-dione (1.81 g, 7.00 mmol, 1.3 equiv.) in pyridine (20 mL) was added EDCI (1.55 g, 8.08 mmol, 1.5 equiv.). The mixture was stirred at 40° C. for 16 hours. The reaction mixture was diluted with water (20 mL) and extracted with EtOAc (60 mL). The combined organic layers were dried over sodium sulfate, filtered, and concentrated under reduced pressure to give a residue, which was purified by flash silica gel chromatography (89% ethyl acetate/petroleum ether) to afford 2-[4-[3-(4-bromo-3-methyl-phenoxy)propyl]-1-piperidyl]-N-[3-(2,6-dioxo-3-piperidyl)-1-methyl-indazol-6-yl]acetamide (1.3 g) as a yellow solid.

### Step 6: N-[3-(2,6-dioxo-3-piperidyl)-1-methyl-indazol-6-yl]-2-[4-[3-[3-methyl-4-(4,4,5,5-tetramethyl-1,3,2-dioxaborolan-2-yl)phenoxy]propyl]-1-piperidyl]acetamide

A mixture of 2-[4-[3-(4-bromo-3-methyl-phenoxy)propyl]-1-piperidyl]-N-[3-(2,6-dioxo-3-piperidyl)-1-methyl-indazol-6-yl]acetamide (625 mg, 1.02 mmol, 1 equiv.), bis(pinacolato)diboron (519.90 mg, 2.05 mmol, 2 equiv.), KOAc (301.40 mg, 3.07 mmol, 3 equiv.), and Pd(dppf)Cl₂·CH₂Cl₂ (83.60 mg, 102.37 mol, 0.1 equiv.) in dioxane (11 mL) was degassed and purged with N₂ three times. The mixture was stirred at 90° C. for 16 hours under a N₂ atmosphere. The reaction mixture was concentrated under reduced pressure to give a residue, which was purified by flash silica gel chromatography (78% ethyl acetate/petroleum ether) and reverse-phase HPLC to afford N-[3-(2,6-dioxo-3-piperidyl)-1-methyl-indazol-6-yl]-2-[4-[3-[3-methyl-4-(4,4,5,5-tetramethyl-1,3,2-dioxaborolan-2-yl)phenoxy]propyl]-1-piperidyl]acetamide (730 mg, crude, FA) as a brown solid.

LC-MS (ESI): m/z=658.5 [M+H]⁺ 1H NMR (400 MHz, DMSO-d₆) δ=10.88 (s, 1H), 9.84 (s, 1H), 8.15 (s, 1H), 8.05 (s, 1H), 7.63 (d, J=8.8 Hz, 1H), 7.55 (d, J=8.0 Hz, 1H), 7.22 (d, J=8.8 Hz, 1H), 6.77-6.62 (m, 2H), 4.32 (dd, J=5.2, 9.6 Hz, 1H), 3.96 (s, 2H), 3.92 (s, 3H), 3.16 (d, J=6.8 Hz, 5H), 2.89 (d, J=10.4 Hz, 2H), 2.76-2.54 (m, 3H), 2.42 (s, 2H), 2.40-2.30 (m, 2H), 2.17 (dd, J=5.6, 12.8 Hz, 3H), 1.70 (dd, J=7.6, 12.4 Hz, 4H), 1.47-1.15 (m, 13H).

### Step 7: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylate

A mixture of N-[3-(2,6-dioxo-3-piperidyl)-1-methyl-indazol-6-yl]-2-[4-[3-[3-methyl-4-(4,4,5,5-tetramethyl-1,3,2-dioxaborolan-2-yl)phenoxy]propyl]-1-piperidyl]acetamide (50 mg, 76.03 mol, 1 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (63.57 mg, 91.24 mol, 1.2 equiv.), KF (1.5 M, 152.07 μL, 3 equiv.), and Ad₂nBup Pd G₃ (55.37 mg, 76.03 mol, 1 equiv.) in dioxane (1.5 mL) was degassed and purged with N₂ three times. The mixture was stirred at 100° C. for 3 hours under a N₂ atmosphere. The solution was concentrated under reduced pressure to give a residue, which was purified by flash silica gel chromatography (72% ethyl acetate/petroleum ether) and reverse-phase HPLC to afford tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylate (230 mg) as a yellow solid.

### Step 8: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylate (230 mg, 200.44 mol, 1 equiv.) in TFA (2 mL) and DCM (1 mL) was stirred at 40° C. for 2 hours. The solution was concentrated under reduced pressure to give a residue, which was purified by prep-HPLC to afford 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid (49.15 mg) as a white solid.

LC-MS (ESI): m/z=481.3 [M/2+H]⁺. 1H NMR (400 MHz, DMSO-d6) δ=12.84-12.10 (m, 2H), 10.88 (s, 1H), 9.83 (s, 1H), 8.57 (d, J=5.6 Hz, 1H), 8.10-7.98 (m, 2H), 7.82 (d, J=8.0 Hz, 1H), 7.68-7.55 (m, 2H), 7.54-7.43 (m, 2H), 7.37 (t, J=8.0 Hz, 1H), 7.22 (d, J=8.4 Hz, 1H), 7.09-7.01 (m, 1H), 6.94 (d, J=8.8 Hz, 1H), 6.81 (s, 1H), 6.76-6.67 (m, 1H), 5.24 (s, 2H), 4.39-4.26 (m, 1H), 4.00-3.88 (m, 7H), 3.14 (s, 2H), 3.08 (d, J=4.0 Hz, 2H), 2.94-2.83 (m, 2H), 2.68-2.62 (m, 2H), 2.17 (dd, J=4.4, 11.6 Hz, 3H), 2.05 (s, 3H), 1.79-1.66 (m, 4H), 1.47-1.17 (m, 6H).

### Example 14: 6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid] (Compound 133b)

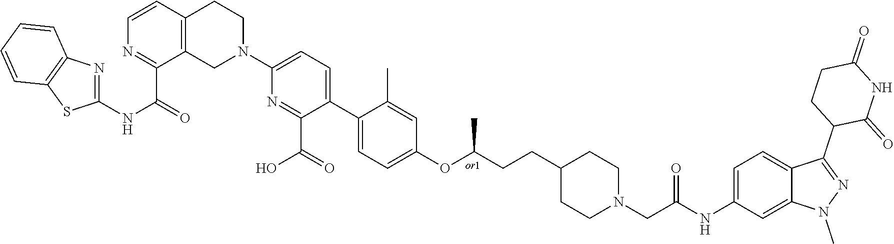 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O]

### Step 1: tert-butyl 4-(3-(methoxy(methyl)amino)-3-oxopropyl)piperidine-1-carboxylate

To a solution of 3-(1-(tert-butoxycarbonyl)piperidin-4-yl)propanoic acid (25 g, 97.15 mmol, 1 equiv.) in DMF (300 mL) was added HATU (55.41 g, 145.73 mmol, 1.5 equiv.), DIPEA (37.67 g, 291.46 mmol, 50.77 mL, 3 equiv.), and N-methoxymethanamine (18.95 g, 194.31 mmol, 2 equiv. HCl). The mixture was stirred at 25° C. for 12 hours. The reaction mixture was diluted with H₂O (600 mL) and extracted with EtOAc (1200 mL). The combined organic layers were washed with brine (400 mL), dried over sodium sulfate, filtered, and concentrated under reduced pressure to give a residue, which was purified by flash silica gel chromatography (0-43% ethyl acetate/petroleum ether) to afford tert-butyl 4-(3-(methoxy(methyl)amino)-3-oxopropyl)piperidine-1-carboxylate (24 g) as a yellow oil.

¹H NMR (400 MHz, DMSO-d₆) δ=3.91 (d, J=12.0 Hz, 2H), 3.65 (s, 3H), 3.07 (s, 3H), 2.71-2.56 (m, 2H), 2.38 (t, J=7.2 Hz, 2H), 1.62 (d, J=12.4 Hz, 2H), 1.48-1.40 (m, 3H), 1.38 (s, 9H), 1.03-0.88 (m, 2H).

### Step 2: tert-butyl 4-(3-oxobutyl)piperidine-1-carboxylate

A mixture of tert-butyl 4-(3-(methoxy(methyl)amino)-3-oxopropyl)piperidine-1-carboxylate (24 g, 79.90 mmol, 1 equiv.) in THE (500 mL) was degassed and purged with N₂ three times followed by the addition of MeMgBr (3 M, 159.79 mL, 6 equiv.) at −10° C. The mixture was stirred at 25° C. for 2 hours under a N₂ atmosphere. The reaction mixture was poured into saturated aqueous ammonium chloride (600 mL) and extracted with EtOAc (1200 mL). The combined organic layers were washed with brine (500 mL), dried over anhydrous sodium sulfate, filtered, and concentrated under reduced pressure to give a residue, which was purified by flash silica gel chromatography (0-20% ethyl acetate/petroleum ether) to afford tert-butyl 4-(3-oxobutyl)piperidine-1-carboxylate (14 g) as a colorless oil.

¹H NMR (400 MHz, DMSO-d₆) δ=3.90 (d, J=12.0 Hz, 2H), 2.63 (s, 2H), 2.44 (t, J=7.4 Hz, 2H), 2.07 (s, 3H), 1.59 (d, J=12.4 Hz, 2H), 1.42-1.27 (m, 12H), 1.00-0.84 (m, 2H).

### Step 3: tert-butyl 4-(3-hydroxybutyl)piperidine-1-carboxylate

To a solution of tert-butyl 4-(3-oxobutyl)piperidine-1-carboxylate (14 g, 49.34 mmol, 1 equiv.) in EtOH (300 mL) was added NaBH₄ (2.24 g, 59.21 mmol, 1.2 equiv.). The mixture was stirred at 0° C. for 1 hour. The reaction mixture was poured into saturated aqueous ammonium chloride (500 mL) and extracted with EtOAc (1500 mL). The combined organic layers were washed with brine (500 mL), dried over anhydrous sodium sulfate, filtered, and concentrated under reduced pressure to afford tert-butyl 4-(3-hydroxybutyl)piperidine-1-carboxylate (14 g, crude) as a yellow oil.

¹H NMR (400 MHz, DMSO-d₆,) 6=4.20-3.97 (m, 2H), 3.84-3.68 (m, 1H), 2.66 (s, 2H), 1.65 (d, J=12.0 Hz, 2H), 1.45 (s, 9H), 1.42-1.30 (m, 3H), 1.30-1.16 (m, 4H), 1.15-0.99 (m, 3H).

### Step 4: tert-butyl 4-(3-(4-bromo-3-methylphenoxy)butyl)piperidine-1-carboxylate

A mixture of 4-bromo-3-methylphenol (12.21 g, 65.28 mmol, 1.2 equiv.), tert-butyl 4-(3-hydroxybutyl)piperidine-1-carboxylate (14 g, 54.40 mmol, 1 equiv.), and 2-(tributyl-phosphanylidene)acetonitrile (19.69 g, 81.60 mmol, 1.5 equiv.) in toluene (300 mL) was degassed and purged with N₂ three times. The mixture was stirred at 120° C. for 12 hours under a N₂ atmosphere. The reaction mixture was filtered and concentrated under reduced pressure to give a residue, which was purified by revise-phase prep-HPLC to afford tert-butyl 4-(3-(4-bromo-3-methylphenoxy)butyl)piperidine-1-carboxylate (16 g) as a brown oil.

¹H NMR (400 MHz, DMSO-d₆) δ=7.41 (d, J=8.8 Hz, 1H), 6.92 (d, J=3.2 Hz, 1H), 6.70 (dd, J=2.8, 8.8 Hz, 1H), 4.39 (q, J=5.6 Hz, 1H), 3.90 (d, J=11.2 Hz, 2H), 2.71-2.59 (m, 2H), 2.28 (s, 3H), 1.67-1.57 (m, 3H), 1.57-1.49 (m, 1H), 1.38 (s, 9H), 1.36-1.27 (m, 2H), 1.25-1.22 (m, 1H), 1.20 (d, J=6.0 Hz, 3H), 1.00-0.87 (m, 2H).

### Step 5: tert-butyl (S)-4-(3-(4-bromo-3-methylphenoxy)butyl)piperidine-1-carboxylate and tert-butyl (R)-4-(3-(4-bromo-3-methylphenoxy)butyl)piperidine-1-carboxylate

Enantiomers of tert-butyl 4-(3-(4-bromo-3-methylphenoxy)butyl)piperidine-1-carboxylate were further separated by SFC (column: Daicel Chiralpak AY (250 mm×50 mm, 10 μm); mobile phase: [CO₂-MeOH (0.1% NH₃·H₂O)]; B %: 15%, isocratic elution mode) to give tert-butyl (R)-4-(3-(4-bromo-3-methylphenoxy)butyl)piperidine-1-carboxylate (Peak 1) (1 g) as a yellow oil and tert-butyl (S)-4-(3-(4-bromo-3-methylphenoxy)butyl)piperidine-1-carboxylate (Peak 2) (1 g) as a yellow oil.

Peak 1:

SFC: t_{R}=0.860 mins

¹H NMR (400 MHz, DMSO-d₆) δ=7.41 (d, J=8.8 Hz, 1H), 6.92 (s, 1H), 6.70 (d, J=8.8 Hz, 1H), 4.42-4.35 (m, 1H), 3.90 (d, J=11.6 Hz, 2H), 2.82-2.53 (m, 2H), 2.28 (s, 3H), 1.66-1.48 (m, 4H), 1.38 (s, 11H), 1.26-1.14 (m, 4H), 1.02-0.83 (m, 2H).

Peak 2:

SFC: t_{R}=1.052 mins

¹H NMR (400 MHz, DMSO-d₆) δ=7.41 (d, J=8.8 Hz, 1H), 6.92 (s, 1H), 6.74-6.65 (m, 1H), 4.48-4.31 (m, 1H), 3.90 (d, J=11.6 Hz, 2H), 2.77-2.56 (m, 2H), 2.28 (s, 3H), 1.66-1.50 (m, 4H), 1.42-1.28 (m, 11H), 1.26-1.17 (m, 4H), 1.00-0.86 (m, 2H).

### Step 6: (S)-4-(3-(4-bromo-3-methylphenoxy)butyl)piperidine

A solution of tert-butyl (S)-4-(3-(4-bromo-3-methylphenoxy)butyl)piperidine-1-carboxylate (1 g, 2.35 mmol, 1 equiv.) in HCl/dioxane (4 M, 586.32 μL, 1 equiv.) was stirred at 25° C. for 1 hour. The reaction mixture was filtered and concentrated under reduced pressure to afford (S)-4-(3-(4-bromo-3-methylphenoxy)butyl)piperidine (900 mg, crude, HCl) as a yellow oil.

LC-MS (ESI): m/z=325.9 [M+H]⁺

### Step 7: ethyl (S)-2-(4-(3-(4-bromo-3-methylphenoxy)butyl)piperidin-1-yl)acetate

To a solution of (S)-4-(3-(4-bromo-3-methylphenoxy)butyl)piperidine (900 mg, 2.48 mmol, 1 equiv. HCl) and ethyl 2-bromoacetate (414.36 mg, 2.48 mmol, 274.41 μL, 1 equiv.) in DMF (10 mL) was added potassium carbonate (1.03 g, 7.44 mmol, 3 equiv.). The mixture was stirred at 40° C. for 1 hour. The reaction mixture was diluted with H₂O (50 mL) and extracted with ethyl acetate (90 mL). The combined organic layers were washed with brine (60 mL), dried over sodium sulfate, filtered, and concentrated under reduced pressure to afford ethyl (S)-2-(4-(3-(4-bromo-3-methylphenoxy)butyl)piperidin-1-yl)acetate (1 g, crude) as a yellow oil.

¹H NMR (400 MHz, DMSO-d₆) δ=7.40 (d, J=8.8 Hz, 1H), 6.92 (s, 1H), 6.69 (d, J=8.8 Hz, 1H), 4.44-4.31 (m, 1H), 4.06 (q, J=7.2 Hz, 2H), 3.14 (s, 2H), 2.77 (d, J=10.6 Hz, 2H), 2.28 (s, 3H), 2.08 (t, J=10.8 Hz, 2H), 1.65-1.48 (m, 4H), 1.35-1.27 (m, 1H), 1.25-1.05 (m, 10H).

### Step 8: (S)-2-(4-(3-(4-bromo-3-methylphenoxy)butyl)piperidin-1-yl)acetic acid

To a solution of ethyl (S)-2-(4-(3-(4-bromo-3-methylphenoxy)butyl)piperidin-1-yl)acetate (1 g, 2.43 mmol, 1 equiv.) in THF (10 mL) and H₂O (2 mL) was added LiOH·H₂O (305.27 mg, 7.28 mmol, 3 equiv.). The mixture was stirred at 40° C. for 12 hours. The reaction mixture was concentrated under reduced pressure to remove THF. The aqueous phase was adjusted to pH=4-5 with 1M HCl. The reaction mixture was filtered. The filter cake was diluted in ethyl acetate. The combined organic layers were dried over sodium sulfate, filtered, and concentrated under reduced pressure to afford (S)-2-(4-(3-(4-bromo-3-methylphenoxy)butyl)piperidin-1-yl)acetic acid (700 mg, crude) as a yellow solid.

¹H NMR (400 MHz, DMSO-d₆) δ=7.41 (d, J=8.8 Hz, 1H), 6.92 (s, 1H), 6.70 (d, J=7.2 Hz, 1H), 4.40 (d, J=5.6 Hz, 1H), 3.77 (s, 2H), 3.41 (d, J=10.4 Hz, 2H), 2.96 (s, 2H), 2.28 (s, 3H), 1.77 (s, 2H), 1.63-1.50 (m, 2H), 1.45 (s, 2H), 1.36-1.13 (m, 6H).

### Step 9: 2-(4-((S)-3-(4-bromo-3-methylphenoxy)butyl)piperidin-1-yl)-N-(3-(2,6-dioxopiperidin-3-yl)-1-methyl-1H-indazol-6-yl)acetamide

To a solution of (S)-2-(4-(3-(4-bromo-3-methylphenoxy)butyl)piperidin-1-yl)acetic acid (700 mg, 1.82 mmol, 1 equiv.) and 3-(6-amino-1-methyl-indazol-3-yl)piperidine-2,6-dione (644.22 mg, 2.19 mmol, 1.2 equiv. HCl) in pyridine (15 mL) was added EDCI (523.76 mg, 2.73 mmol, 1.5 equiv.). The mixture was stirred at 40° C. for 1 hour. The reaction mixture was quenched by the addition of H₂O (10 mL), diluted with ethyl acetate (30 mL), and extracted with ethyl acetate (30 mL). The combined organic layers were washed with brine (30 mL), dried over sodium sulfate, filtered, and concentrated under reduced pressure to give a residue, which was purified by flash silica gel chromatography (0~100% ethyl acetate/petroleum ether) to afford 2-(4-((S)-3-(4-bromo-3-methylphenoxy)butyl)piperidin-1-yl)-N-(3-(2,6-dioxopiperidin-3-yl)-1-methyl-1H-indazol-6-yl)acetamide (700 mg) as a yellow solid.

LC-MS (ESI): m/z=624.1 [M+H]⁺

### Step 10: N-(3-(2,6-dioxopiperidin-3-yl)-1-methyl-1H-indazol-6-yl)-2-(4-((S)-3-(3-methyl-4-(4,4,5,5-tetramethyl-1,3,2-dioxaborolan-2-yl)phenoxy)butyl)piperidin-1-yl)acetamide

A mixture of 2-(4-((S)-3-(4-bromo-3-methylphenoxy)butyl)piperidin-1-yl)-N-(3-(2,6-dioxopiperidin-3-yl)-1-methyl-1H-indazol-6-yl)acetamide (650 mg, 1.04 mmol, 1 equiv.), 4,4,5,5-tetramethyl-2-(4,4,5,5-tetramethyl-1,3,2-dioxaborolan-2-yl)-1,3,2-dioxaborolane (396.42 mg, 1.56 mmol, 1.5 equiv.), and KOAc (306.42 mg, 3.12 mmol, 3 equiv.) in dioxane (10 mL) was degassed and purged with N₂ three times. To this mixture was added Pd(dppf)Cl₂·CH₂Cl₂ (84.99 mg, 104.07 mol, 0.1 equiv.). The mixture was stirred at 90° C. for 12 hours under a N₂ atmosphere. The reaction mixture was filtered and concentrated under reduced pressure to give a residue, which was purified by flash silica gel chromatography (0-100% ethyl acetate/petroleum ether) to afford N-(3-(2,6-dioxopiperidin-3-yl)-1-methyl-1H-indazol-6-yl)-2-(4-((S)-3-(3-methyl-4-(4,4,5,5-tetramethyl-1,3,2-dioxaborolan-2-yl)phenoxy)butyl)piperidin-1-yl)acetamide (600 mg) as a red solid.

LC-MS (ESI): m/z=672.2 [M+H]⁺

### Step 11: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate

N-(3-(2,6-dioxopiperidin-3-yl)-1-methyl-1H-indazol-6-yl)-2-(4-((S)-3-(3-methyl-4-(4,4,5,5-tetramethyl-1,3,2-dioxaborolan-2-yl)phenoxy)butyl)piperidin-1-yl)acetamide (210 mg, 312.67 mol, 1 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (261.42 mg, 375.21 mol, 1.2 equiv.), KF (54.50 mg, 938.01 mol, 21.97 μL, 3 equiv.), and Ad₂nBuP Pd G₃ (227.71 mg, 312.67 mol, 1 equiv.) were taken up into a microwave tube in dioxane (10 mL) and H₂O (1 mL) under a N₂ atmosphere. The sealed tube was heated at 115° C. for 2 hours under a microwave reactor. The reaction mixture was filtered and concentrated under reduced pressure to give a residue, which was purified by flash silica gel chromatography (0~81% ethyl acetate/petroleum ether) to afford tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate (150 mg) as a yellow solid.

### Step 12: 6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

To a solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate (150 mg, 102.02 mol, 1 equiv.) in DCM (3 mL) was added TFA (4.61 g, 40.39 mmol, 3 mL, 395.85 equiv.). The mixture was stirred at 25° C. for 12 hours. The reaction mixture was filtered and concentrated under reduced pressure to give a residue, which was purified by prep-HPLC to afford 6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid] (48.23 mg) as a yellow solid.

LC-MS (ESI): m/z=975.4 [M+H]⁺

¹H NMR (400 MHz, DMSO-d₆) δ=13.03-12.44 (m, 1H), 12.31 (s, 1H), 10.88 (s, 1H), 10.15-9.46 (m, 1H), 8.57 (d, J=4.8 Hz, 1H), 8.09-7.98 (m, 2H), 7.82 (d, J=8.0 Hz, 1H), 7.65 (d, J=8.4 Hz, 1H), 7.58 (d, J=4.4 Hz, 1H), 7.53-7.44 (m, 2H), 7.40-7.32 (m, 1H), 7.19 (d, J=8.4 Hz, 1H), 7.05 (d, J=8.8 Hz, 1H), 6.93 (d, J=8.4 Hz, 1H), 6.78 (s, 1H), 6.72 (d, J=8.8 Hz, 1H), 5.24 (s, 2H), 4.50-4.38 (m, 1H), 4.33 (dd, J=4.8, 9.2 Hz, 1H), 3.97 (t, J=5.2 Hz, 2H), 3.93 (s, 3H), 3.26-2.79 (m, 6H), 2.67-2.61 (m, 2H), 2.43-2.26 (m, 2H), 2.25-2.10 (m, 2H), 2.04 (s, 3H), 1.79-1.54 (m, 4H), 1.48-1.26 (m, 5H), 1.24 (d, J=5.6 Hz, 3H).

### Example 15: 6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid]

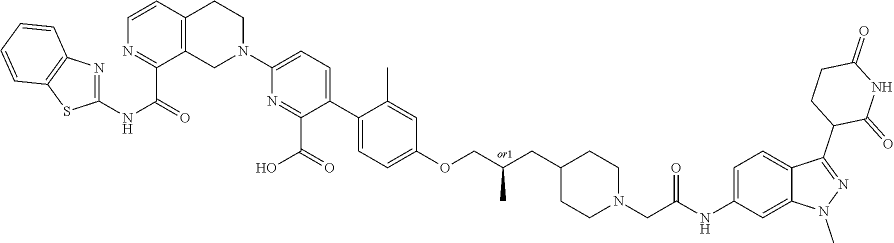 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O]

### Step 1: tert-butyl 4-(3-ethoxy-2-methyl-3-oxo-propyl)piperidine-1-carboxylate

To a solution of tert-butyl 4-[(E)-3-ethoxy-2-methyl-3-oxo-prop-1-enyl]piperidine-1-carboxylate (3.0 g, 10.09 mmol, 1.0 equiv.) in MeOH (30 mL) was added Pd on carbon (10 wt %, 2.15 g, 2.02 mmol, 0.2 equiv.) and Pd(OH)₂ on carbon (20 wt %, 1.42 g, 2.02 mmol, 0.2 equiv.) under a N₂ atmosphere. The suspension was degassed and purged with H₂ three times. The mixture was stirred under H₂ (15 psi) at 40° C. for 16 hours. The reaction mixture was filtered and concentrated under reduced pressure to give tert-butyl 4-(3-ethoxy-2-methyl-3-oxo-propyl)piperidine-1-carboxylate (8 g) as a colorless oil.

LC-MS (ESI): m/z=200.0 [M+H−100]⁺.

### Step 2: tert-butyl 4-(3-hydroxy-2-methyl-propyl)piperidine-1-carboxylate

To a solution of tert-butyl 4-(3-ethoxy-2-methyl-3-oxo-propyl)piperidine-1-carboxylate (2.8 g, 9.35 mmol, 1.0 equiv.) in THF (30 mL) was added LAH (2.5 M, 4.49 mL, 1.2 equiv.) at 0° C. The mixture was stirred at 25° C. for 1 hour. To the reaction mixture was added sodium sulfate·10 H₂O 10.0 g at 0° C. and filtered to give a filtrate. The filtrate was concentrated to give tert-butyl 4-(3-hydroxy-2-methyl-propyl)piperidine-1-carboxylate (2 g) as a colorless oil

¹H NMR (400 MHz, DMSO-d₆) δ=4.37 (t, J=5.2 Hz, 1H), 3.90 (d, J=11.6 Hz, 2H), 3.25-3.21 (m, 1H), 3.17-3.12 (m, 1H), 2.67 (d, J=2.0 Hz, 2H), 1.62-1.56 (m, 3H), 1.47-1.45 (m, 1H), 1.38 (s, 9H), 1.26-1.19 (m, 1H), 0.95-0.87 (m, 3H), 0.81 (d, J=6.4 Hz, 3H)

### Step 3: tert-butyl 4-[3-(4-bromo-3-methyl-phenoxy)-2-methyl-propyl]piperidine-1-carboxylate

To a solution of tert-butyl 4-(3-hydroxy-2-methyl-propyl)piperidine-1-carboxylate (1.0 g, 3.89 mmol, 1.0 equiv.) in toluene (20 mL) was added 2-(tributyl-phosphanylidene)acetonitrile (1.41 g, 5.83 mmol, 1.5 equiv.) and 4-bromo-3-methyl-phenol (799.39 mg, 4.27 mmol, 1.1 equiv.). The mixture was stirred at 120° C. for 12 hours. The reaction mixture was concentrated under reduced pressure to give a residue, which was purified by column chromatography (SiO₂, petroleum ether/ethyl acetate=100/1 to 10/1) and reversed-phase HPLC to give tert-butyl 4-[3-(4-bromo-3-methyl-phenoxy)-2-methyl-propyl]piperidine-1-carboxylate (1.2 g) as a yellow oil.

LC-MS (ESI): m/z=326.0 [M+H]⁺

¹H NMR (400 MHz, CDCl₃) δ=7.39 (d, J=8.8 Hz, 1H), 6.78 (s, 1H), 6.60 (d, J=8.8 Hz, 1H), 4.09 (d, J=13.2 Hz, 2H), 3.76-3.67 (m, 2H), 2.74-2.65 (m, 2H), 2.36 (s, 3H), 2.06-2.03 (m, 1H), 1.67 (d, J=12.0 Hz, 2H), 1.57-1.52 (m, 2H), 1.46 (s, 9H), 1.43-1.39 (m, 1H), 1.18-1.05 (m, 3H), 1.02 (d, J=6.4 Hz, 3H)

### Step 4: tert-butyl (R)-4-(3-(4-bromo-3-methylphenoxy)-2-methylpropyl)piperidine-1-carboxylate and tert-butyl (S)-4-(3-(4-bromo-3-methylphenoxy)-2-methylpropyl)piperidine-1-carboxylate

Enantiomers of tert-butyl 4-[3-(4-bromo-3-methyl-phenoxy)-2-methyl-propyl]piperidine-1-carboxylate were separated by SFC (column: Daicel Chiralpak IC (250 mm×30 mm, 10 μm); mobile phase: [CO₂-iPrOH(0.1% NH₃·H₂O)]; B %:15%, isocratic elution mode) to give tert-butyl (S)-4-(3-(4-bromo-3-methylphenoxy)-2-methylpropyl)piperidine-1-carboxylate (Peak 1) (950 mg) as a colorless oil and tert-butyl (R)-4-(3-(4-bromo-3-methylphenoxy)-2-methylpropyl)piperidine-1-carboxylate (Peak 2) (950 mg) as a colorless oil.

Peak 1:

SFC: t_{R}=1.430 min

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 3.91 (d, J=12.0 Hz, 2H), 3.89-3.77 (m, 1H), 2.67 (s, 2H), 2.29 (s, 3H), 2.01-1.96 (m, 1H), 1.62 (d, J=12.8 Hz, 1H), 1.56-1.49 (m, 2H), 1.38-1.32 (m, 10H), 1.12-1.11 (m, 1H), 0.95 (d, J=6.8 Hz, 3H), 0.70-0.62 (m, 1H)

Peak 2:

SFC: t_{R}=1.788 min

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 3.91 (d, J=12.0 Hz, 2H), 3.89-3.77 (m, 1H), 2.67 (s, 2H), 2.29 (s, 3H), 2.01-1.96 (m, 1H), 1.62 (d, J=12.8 Hz, 1H), 1.56-1.49 (m, 2H), 1.38-1.32 (m, 10H), 1.12-1.11 (m, 1H), 0.95 (d, J=6.8 Hz, 3H), 0.70-0.62 (m, 1H)

### Step 5: (R)-4-(3-(4-bromo-3-methylphenoxy)-2-methylpropyl)piperidine

A solution of tert-butyl (R)-4-(3-(4-bromo-3-methylphenoxy)-2-methylpropyl)piperidine-1-carboxylate (950 mg, 2.23 mmol, 1.0 equiv.) in HCl/dioxane (10 mL) and EtOAc (2 mL) was stirred at 25° C. for 1 hour. The reaction mixture was concentrated under reduced pressure to give (R)-4-(3-(4-bromo-3-methylphenoxy)-2-methylpropyl)piperidine (790 mg) as a white solid.

### Step 6: ethyl (R)-2-(4-(3-(4-bromo-3-methylphenoxy)-2-methylpropyl)piperidin-1-yl)acetate

To a solution of (R)-4-(3-(4-bromo-3-methylphenoxy)-2-methylpropyl)piperidine (790 mg, 2.42 mmol, 1.0 equiv.) in DMF (10 mL) was added potassium carbonate (1.00 g, 7.26 mmol, 3.0 equiv.) and ethyl 2-bromoacetate (404.36 mg, 2.42 mmol, 267.97 μL, 1.0 equiv.). The mixture was stirred at 40° C. for 1 hour. The reaction was diluted with H₂O (50 mL) and extracted with ethyl acetate (90 mL). The combined organic phase was washed with brine (50 mL), dried over anhydrous sodium sulfate, filtered, and concentrated under reduced pressure to give ethyl (R)-2-(4-(3-(4-bromo-3-methylphenoxy)-2-methylpropyl)piperidin-1-yl)acetate (900 mg) as a yellow solid.

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 4.07 (q, J=7.2 Hz, 2H), 3.80-3.76 (m, 1H), 3.72-3.68 (m, 1H), 3.15 (s, 2H), 2.79 (d, J=10.8 Hz, 2H), 2.29 (s, 3H), 2.16-2.08 (m, 2H), 1.97-1.92 (m, 1H), 1.60 (d, J=11.2 Hz, 2H), 1.37-1.26 (m, 2H), 1.18 (q, J=7.2 Hz, 3H), 1.14-0.99 (m, 3H), 0.94 (d, J=6.8 Hz, 3H)

### Step 7: 2-[4-[(2R)-3-(4-bromo-3-methyl-phenoxy)-2-methyl-propyl]-1-piperidyl]acetic acid

To a solution of ethyl (R)-2-(4-(3-(4-bromo-3-methylphenoxy)-2-methylpropyl)piperidin-1-yl)acetate (900 mg, 2.18 mmol, 1.0 equiv.) in H₂O (1 mL) and THE (5 mL) was added LiOH·H₂O (274.76 mg, 6.55 mmol, 3.0 equiv.). The mixture was stirred at 40° C. for 1 hour. The mixture was adjusted to pH=3 with 1M HCl. The reaction was diluted with H₂O (50 mL) and extracted with ethyl acetate (150 mL). The combined organic phase was washed with brine (50 mL), dried over anhydrous sodium sulfate, filtered, and concentrated under reduced pressure to give 2-[4-[(2R)-3-(4-bromo-3-methyl-phenoxy)-2-methyl-propyl]-1-piperidyl]acetic acid (840 mg, crude) as a yellow solid.

### Step 8: 2-[4-[(2R)-3-(4-bromo-3-methyl-phenoxy)-2-methyl-propyl]-1-piperidyl]-N-[3-(2,6-dioxo-3-piperidyl)-1-methyl-indazol-6-yl]acetamide

To a solution of 2-[4-[(2R)-3-(4-bromo-3-methyl-phenoxy)-2-methyl-propyl]-1-piperidyl]acetic acid (840 mg, 2.19 mmol, 1.0 equiv.) and 3-(6-amino-1-methyl-indazol-3-yl)piperidine-2,6-dione (677.43 mg, 2.62 mmol, 1.2 equiv.) in pyridine (5 mL) was added EDCI (628.52 mg, 3.28 mmol, 1.5 equiv.). The mixture was stirred at 40° C. for 1 hour. The reaction mixture was concentrated under reduced pressure to give a residue, which was diluted with H₂O (50 mL) and extracted with ethyl acetate (90 mL). The combined organic phase was washed with brine (50 mL), dried over anhydrous sodium sulfate, filtered, and concentrated under reduced pressure to give a residue, which was purified by flash silica gel chromatography (0-10% MeOH/DCM) to give 2-[4-[(2R)-3-(4-bromo-3-methyl-phenoxy)-2-methyl-propyl]-1-piperidyl]-N-[3-(2,6-dioxo-3-piperidyl)-1-methyl-indazol-6-yl]acetamide (800 mg) as a yellow solid.

### Step 9: N-[3-(2,6-dioxo-3-piperidyl)-1-methyl-indazol-6-yl]-2-[4-[(2R)-2-methyl-3-[3-methyl-4-(4,4,5,5-tetramethyl-1,3,2-dioxaborolan-2-yl)phenoxy]propyl]-1-piperidyl]acetamide

A mixture of 2-[4-[(2R)-3-(4-bromo-3-methyl-phenoxy)-2-methyl-propyl]-1-piperidyl]-N-[3-(2,6-dioxo-3-piperidyl)-1-methyl-indazol-6-yl]acetamide (800 mg, 1.28 mmol, 1.0 equiv.), bis(pinacolato)diboron (650.53 mg, 2.56 mmol, 2.0 equiv.), and Pd(dppf)Cl₂ (93.72 mg, 128.09 mol, 0.1 equiv.), KOAc (377.13 mg, 3.84 mmol, 3.0 equiv.) in dioxane (15 mL) was degassed and purged with N₂ three times. The mixture was stirred at 90° C. for 16 hours under a N₂ atmosphere. The reaction mixture was filtered and concentrated under reduced pressure to give a residue, which was purified by prep-HPLC to give N-[3-(2,6-dioxo-3-piperidyl)-1-methyl-indazol-6-yl]-2-[4-[(2R)-2-methyl-3-[3-methyl-4-(4,4,5,5-tetramethyl-1,3,2-dioxaborolan-2-yl)phenoxy]propyl]-1-piperidyl]acetamide (420 mg) as a white solid.

### Step 10: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate

A mixture of N-[3-(2,6-dioxo-3-piperidyl)-1-methyl-indazol-6-yl]-2-[4-[(2R)-2-methyl-3-[3-methyl-4-(4,4,5,5-tetramethyl-1,3,2-dioxaborolan-2-yl)phenoxy]propyl]-1-piperidyl]acetamide (200 mg, 297.78 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (248.97 mg, 357.34 mol, 1.2 equiv.), KF (51.90 mg, 893.35 mol, 20.93 μL, 3.0 equiv.), and Ad₂nBuP Pd G₃ (216.87 mg, 297.78 mol, 1.0 equiv.) in dioxane (10 mL) and H₂O (0.5 mL) was degassed and purged with N₂ three times. The mixture was stirred at 115° C. for 2 hours in microwave reactor. The reaction mixture was filtered and concentrated under reduced pressure to give a residue, which was purified by prep-HPLC to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (150 mg) as a yellow solid.

### Step 11: 6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid]

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (150 mg, 129.14 mol, 1.0 equiv.) in DCM (1 mL) and TFA (2 mL) was stirred at 40° C. for 2 hours. The reaction mixture was filtered and concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid] (35.46 mg) as a yellow solid.

LC-MS (ESI): m/z=975.3 [M+H]⁺

¹H NMR (400 MHz, DMSO-d₆) δ=7.43 (d, J=8.8 Hz, 1H), 6.94 (d, J=2.8 Hz, 1H), 6.72 (dd, J=3.2, 8.8, 1H), 4.40 (q, J=6.0, 1H), 4.09 (q, J=7.2 Hz, 2H), 3.16 (s, 2H), 2.82 (d, J=11.2, 2H), 2.31 (s, 3H), 2.11 (t, J=10.4 Hz, 2H), 1.65-1.52 (m, 4H), 1.35-1.31 (m, 1H), 1.23-1.18 (m, 7H), 1.15-1.08 (m, 3H)

### Example 16: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid (Compound 155)

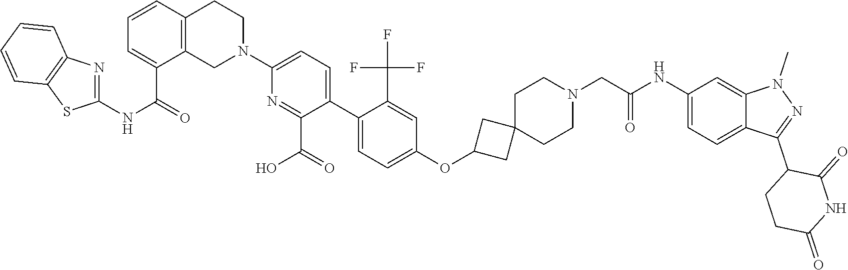 [SMILES: Cn1nc(C2CCC(=O)NC2=O)c2ccc(NC(=O)CN3CCC4(CC3)CC(Oc3ccc(-c5ccc(N6CCc7cccc(C(=O)Nc8nc9ccccc9s8)c7C6)nc5C(=O)O)c(C(F)(F)F)c3)C4)cc21]

### Step 1: tert-butyl 2-[4-bromo-3-(trifluoromethyl)phenoxy]-7-azaspiro[3.5]nonane-7-carboxylate

A mixture of 4-bromo-3-(trifluoromethyl)phenol (1.20 g, 4.97 mmol, 1.2 equiv.), tert-butyl 2-hydroxy-7-azaspiro[3.5]nonane-7-carboxylate (1.0 g, 4.14 mmol, 1.0 equiv.), 2-(tributyl-phosphanylidene)acetonitrile (1.50 g, 6.22 mmol, 1.5 equiv.) in toluene (15 mL) was degassed and purged with N₂ three times. The mixture was stirred at 120° C. for 2 hours in a microwave reactor. The reaction mixture was concentrated under reduced pressure to give a residue, which was purified by flash silica gel chromatography (0-2% ethyl acetate/petroleum ether) to give tert-butyl 2-[4-bromo-3-(trifluoromethyl)phenoxy]-7-azaspiro[3.5]nonane-7-carboxylate (5 g) as a yellow solid.

¹H NMR (400 MHz, DMSO-d₆) δ=7.74 (d, J=8.8 Hz, 1H), 7.22 (d, J=2.8 Hz, 1H), 7.08 (dd, J=8.8, 2.8 Hz, 1H), 4.91-4.79 (m, 1H), 3.30-3.26 (m, 2H), 3.23-3.18 (m, 2H), 2.45-2.38 (m, 2H), 1.84-1.77 (m, 2H), 1.55-1.51 (m, 2H), 1.49-1.45 (m, 2H), 1.38 (s, 9H).

### Step 2: 2-[4-bromo-3-(trifluoromethyl)phenoxy]-7-azaspiro[3.5]nonane

To a solution of tert-butyl 2-[4-bromo-3-(trifluoromethyl)phenoxy]-7-azaspiro[3.5]nonane-7-carboxylate (1.2 g, 2.58 mmol, 1.0 equiv.) in DCM (2 mL) was added HCl/dioxane (2 M, 20 mL). The mixture was stirred at 25° C. for 1 hour. The reaction mixture was concentrated under reduced pressure to give 2-[4-bromo-3-(trifluoromethyl)phenoxy]-7-azaspiro[3.5]nonane (1.0 g) as a white solid.

LC-MS (ESI): m/z=366.0 [M+H]⁺

### Step 3: ethyl 2-[2-[4-bromo-3-(trifluoromethyl)phenoxy]-7-azaspiro[3.5]nonan-7-yl]acetate

To a solution of 2-[4-bromo-3-(trifluoromethyl)phenoxy]-7-azaspiro[3.5]nonane (500 mg, 1.25 mmol, 1.0 equiv., HCl) in DMF (10 mL) was added potassium carbonate (517.42 mg, 3.74 mmol, 3.0 equiv.) and ethyl 2-bromoacetate (208.41 mg, 1.25 mmol, 138.11 μL, 1.0 equiv.). The mixture was stirred at 40° C. for 1 hour. To the reaction mixture was added (100 mL) water and the resulting mixture was extracted with ethyl acetate (200 mL). The combined organic layers were washed with brine (100 mL), dried over anhydrous sodium sulfate, filtered, and concentrated under reduced pressure to give ethyl 2-[2-[4-bromo-3-(trifluoromethyl)phenoxy]-7-azaspiro[3.5]nonan-7-yl]acetate (500 mg) as a yellow oil.

LC-MS (ESI): m/z=450.1 [M+H]⁺

### Step 4: tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-(trifluoromethyl)phenyl)picolinate

To a mixture of ethyl 2-[2-[4-bromo-3-(trifluoromethyl)phenoxy]-7-azaspiro[3.5]nonan-7-yl]acetate (500 mg, 1.11 mmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (748.19 mg, 1.22 mmol, 1.1 equiv.) in dioxane (5 mL) and H₂O (0.5 mL) was added Ad₂nBup Pd G₃ (80.87 mg, 111.04 mol, 0.1 equiv.) and KF (193.53 mg, 3.33 mmol, 3 equiv.). The mixture was stirred at 100° C. for 2 hours. The reaction mixture was concentrated under reduced pressure to give a residue, which was purified by flash silica gel chromatography (0~56% ethyl acetate/petroleum ether) to give tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-(trifluoromethyl)phenyl)picolinate (500 mg) as a yellow solid.

### Step 5: 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-7-azaspiro[3.5]nonan-7-yl)acetic acid

To a solution of tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-(trifluoromethyl)phenyl)picolinate (500 mg, 584.14 mol, 1 equiv.) in THF (3.75 mL) and H₂O (1.25 mL) was added LiOH·H₂O (73.54 mg, 1.75 mmol, 3 equiv.). The mixture was stirred at 25° C. for 12 hours. The reaction mixture was concentrated under reduced pressure to remove THE and give a residue, which was diluted with H₂O (5 mL). The pH was adjusted to pH=4 with HCl (1 M, 0.3 mL), extracted with DCM (30 mL), and dried over anhydrous sodium sulfate, filtered, and concentrated under reduced pressure to give 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-7-azaspiro[3.5]nonan-7-yl)acetic acid (400 mg) as a yellow solid.

### Step 6: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate

To a mixture of 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-7-azaspiro[3.5]nonan-7-yl)acetic acid (120 mg, 144.94 mol, 1 equiv.) and 3-(6-amino-1-methyl-indazol-3-yl)piperidine-2,6-dione (56.15 mg, 217.42 mol, 1.5 equiv.) in pyridine (1.2 mL) was added EDCI (41.68 mg, 217.42 mol, 1.5 equiv.). The mixture was stirred at 25° C. for 1 hour. The reaction mixture was partitioned between DCM (15 mL) and H₂O (3 mL). The organic phase was separated, dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate (140 mg) as a white solid.

LC-MS (ESI): m/z=534.9 [M/2+H]⁺.

### Step 7: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid

A solution of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate (140 mg, 131.07 mol, 1 equiv.) in DCM (2 mL) and TFA (1 mL) was stirred at 25° C. for 12 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid (52.4 mg) as a white solid.

LC-MS (ESI): m/z=1012.2 [M+H]⁺

¹H NMR (400 MHz, DMSO-d₆) δ=12.56 (s, 2H), 10.88 (s, 1H), 9.93-9.68 (m, 1H), 8.06-8.01 (m, 2H), 7.79 (d, J=8.0 Hz, 1H), 7.63 (d, J=8.8 Hz, 2H), 7.50-7.42 (m, 3H), 7.36 (td, J=7.6, 11.6 Hz, 2H), 7.25-7.20 (m, 1H), 7.18-7.13 (m, 1H), 7.12-7.08 (m, 2H), 6.98 (d, J=8.8 Hz, 1H), 5.08-4.93 (m, 2H), 4.90-4.80 (m, 1H), 4.32 (dd, J=5.2, 9.6 Hz, 1H), 3.97-3.89 (m, 5H), 3.13 (s, 2H), 3.03 (t, J=5.6 Hz, 2H), 2.71-2.58 (m, 2H), 2.44 (d, J=6.4 Hz, 5H), 2.40-2.29 (m, 2H), 2.22-2.14 (m, 1H), 1.83 (dd, J=6.4, 12.0 Hz, 2H), 1.74-1.64 (m, 4H).

### Example 17: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid (Compound 170)

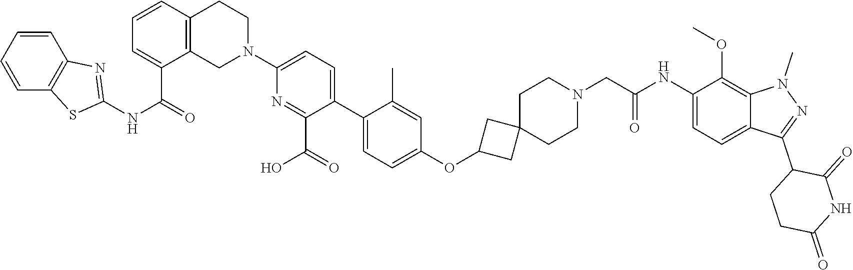 [SMILES: COc1c(NC(=O)CN2CCC3(CC2)CC(Oc2ccc(-c4ccc(N5CCc6cccc(C(=O)Nc7nc8ccccc8s7)c6C5)nc4C(=O)O)c(C)c2)C3)ccc2c(C3CCC(=O)NC3=O)nn(C)c12]

### Step 1: tert-butyl 2-(4-bromo-3-methylphenoxy)-7-azaspiro[3.5]nonane-7-carboxylate

A mixture of tert-butyl 2-hydroxy-7-azaspiro[3.5]nonane-7-carboxylate (1.0 g, 4.14 mmol, 1.0 equiv.), 4-bromo-3-methylphenol (930.03 mg, 4.97 mmol, 1.2 equiv.), 2-(tributyl-phosphanylidene)acetonitrile (1.50 g, 6.22 mmol, 1.5 equiv.) in toluene (10 mL) was degassed and purged with N₂ three times. The mixture was stirred at 120° C. for 2 hours under a N₂ atmosphere in a microwave reactor. The mixture was concentrated under reduced pressure to give a residue, which was purified by reversed-phase HPLC to afford tert-butyl 2-(4-bromo-3-methylphenoxy)-7-azaspiro[3.5]nonane-7-carboxylate (6 g) as a yellow oil.

LC-MS (ESI): m/z=355.0 [M−56+H]⁺.

### Step 2: 2-(4-bromo-3-methylphenoxy)-7-azaspiro[3.5]nonane

A mixture of tert-butyl 2-(4-bromo-3-methylphenoxy)-7-azaspiro[3.5]nonane-7-carboxylate (1.6 g, 3.90 mmol, 1.0 equiv.) in HCl/dioxane (2 M, 16 mL) was stirred at 25° C. for 2 hours. The mixture was concentrated under reduced pressure to afford 2-(4-bromo-3-methylphenoxy)-7-azaspiro[3.5]nonane (1.4 g, crude, HCl) as a gray solid.

### Step 3: ethyl 2-(2-(4-bromo-3-methylphenoxy)-7-azaspiro[3.5]nonan-7-yl)acetate

A mixture of 2-(4-bromo-3-methylphenoxy)-7-azaspiro[3.5]nonane (1.4 g, 4.04 mmol, 1.0 equiv. HCl), ethyl 2-bromoacetate (674.38 mg, 4.04 mmol, 446.91 μL, 1.0 equiv.), and TEA (1.23 g, 12.11 mmol, 1.69 mL, 3.0 equiv.) in acetone (15 mL) was stirred at 25° C. for 2 hours. The mixture was concentrated under reduced pressure to afford ethyl 2-(2-(4-bromo-3-methylphenoxy)-7-azaspiro[3.5]nonan-7-yl)acetate (4 g) as a black oil.

LC-MS (ESI): m/z=396.0 [M+H]⁺

¹H NMR (400 MHz, DMSO-d₆) δ=7.40 (d, J=8.8 Hz, 1H), 6.83 (d, J=2.8 Hz, 1H), 6.60 (dd, J=2.8, 8.4 Hz, 1H), 4.75-4.65 (m, 1H), 4.06 (q, J=7.2 Hz, 2H), 3.13 (s, 2H), 2.43 (s, 2H), 2.38-2.31 (m, 4H), 2.28 (s, 3H), 1.74-1.68 (m, 2H), 1.55 (td, J=5.4, 19.6 Hz, 4H), 1.17 (t, J=7.2 Hz, 3H).

### Step 4: tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-methylphenyl)picolinate

A mixture of ethyl 2-(2-(4-bromo-3-methylphenoxy)-7-azaspiro[3.5]nonan-7-yl)acetate (250 mg, 630.81 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (463.68 mg, 756.97 mol, 1.2 equiv.), Ad₂nBuP Pd G₃ (45.94 mg, 63.08 mol, 0.1 equiv.), and potassium carbonate (1.5 M, 420.54 μL, 1 equiv.) in dioxane (6 mL) was degassed and purged with N₂ three times. The mixture was stirred at 100° C. for 1 hour under a N₂ atmosphere in a microwave reactor. The mixture was concentrated under reduced pressure to give a residue, which was purified by flash silica gel chromatography (0~70% ethyl acetate/petroleum ether) to afford tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-methylphenyl)picolinate (360 mg) as a yellow solid.

### Step 5: 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)oxy)-2-methylphenyl)picolinate (360 mg, 448.88 mol, 1.0 equiv.) and LiOH·H₂O (56.51 mg, 1.35 mmol, 3.0 equiv.) in THF (4 mL) and H₂O (0.8 mL) was stirred at 40° C. for 2 hours. The reaction mixture was diluted with H₂O (5 mL) and concentrated to a turbid liquid. The pH of the turbid liquid was adjusted to pH=3 with 1 N HCl (5 mL) and extracted with DCM/MeOH (10:1) to give 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-7-azaspiro[3.5]nonan-7-yl)acetic acid (330 mg) as a yellow solid.

### Step 6: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate

A mixture of 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-7-azaspiro[3.5]nonan-7-yl)acetic acid (100 mg, 129.21 mol, 1.0 equiv.), 3-(6-amino-7-methoxy-1-methyl-indazol-3-yl)piperidine-2,6-dione (48.43 mg, 167.97 mol, 1.3 equiv.), and EDCI (37.15 mg, 193.81 mol, 1.5 equiv.) in pyridine (2 mL) was stirred at 25° C. for 2 hours. The reaction mixture was diluted with H₂O (5 mL) and extracted with ethyl acetate (15 mL). The combined organic phase was concentrated to give a residue, which was purified by flash silica gel chromatography (0~10% MeOH/DCM) to afford tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate (150 mg, crude) as a brown solid.

### Step 7: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate (150 mg, 143.65 mol, 1.0 equiv.) in DCM (1.5 mL) and TFA (1.5 mL) was stirred at 25° C. for 2 hours. The mixture was concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid (70.44 mg) as an off-white solid.

LC-MS (ESI): m/z=988.3 [M+H]⁺

¹H NMR (400 MHz, DMSO-d₆) δ=13.03-12.36 (m, 2H), 10.89 (s, 1H), 10.00-9.62 (m, 1H), 8.10-7.91 (m, 2H), 7.81-7.77 (m, 1H), 7.62 (d, J=6.8 Hz, 1H), 7.48-7.43 (m, 4H), 7.40-7.34 (m, 2H), 6.93 (dd, J=8.8, 16.4 Hz, 2H), 6.71 (d, J=2.8 Hz, 1H), 6.63 (dd, J=2.8, 8.0 Hz, 1H), 4.97 (s, 2H), 4.77-4.70 (m, 1H), 4.34 (dd, J=5.2, 10.0 Hz, 1H), 4.14 (s, 3H), 3.93-3.87 (m, 5H), 3.48 (d, J=11.6 Hz, 2H), 3.14 (d, J=2.4 Hz, 2H), 3.02 (t, J=5.2 Hz, 2H), 2.71-2.56 (m, 4H), 2.47-2.39 (m, 2H), 2.38-2.37 (m, 1H), 2.38-2.29 (m, 1H), 2.20-2.14 (m, 1H), 2.03 (s, 3H), 1.92-1.79 (m, 3H), 1.77-1.67 (m, 3H).

### Example 18: 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylic acid (Compound 103)

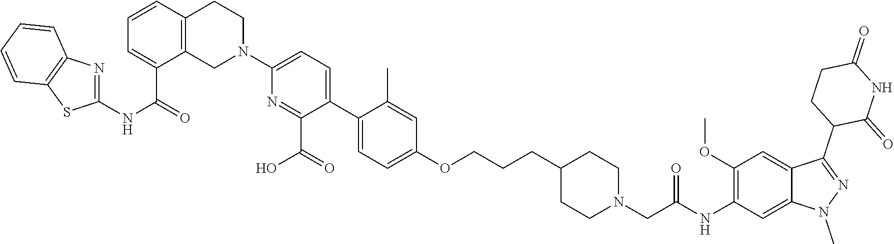 [SMILES: COc1cc2c(C3CCC(=O)NC3=O)nn(C)c2cc1NC(=O)CN1CCC(CCCOc2ccc(-c3ccc(N4CCc5cccc(C(=O)Nc6nc7ccccc7s6)c5C4)nc3C(=O)O)c(C)c2)CC1]

### Step 1: 3-iodo-5-methoxy-6-nitro-1H-indazole

To a solution of 5-methoxy-6-nitro-1H-indazole (4.9 g, 25.37 mmol, 1.0 equiv.) in DMF (2 mL) was added potassium carbonate (7.01 g, 50.74 mmol, 2.0 equiv.) and I₂ (6.44 g, 25.37 mmol, 5.11 mL, 1.0 equiv.). The mixture was stirred at 25° C. for 3 hours. The reaction mixture was poured into water (500 mL), filtered, and concentrated under reduced pressure to give 3-iodo-5-methoxy-6-nitro-1H-indazole (7.6 g, crude), which was used in the next step without further purification.

LC-MS (ESI): m/z=320.0 [M+H]⁺

### Step 2: 3-iodo-5-methoxy-1-methyl-6-nitro-indazole

To a solution of 3-iodo-5-methoxy-6-nitro-1H-indazole (7.5 g, 23.51 mmol, 1.0 equiv.) in DMF (70 mL) was added potassium carbonate (8.12 g, 58.77 mmol, 2.5 equiv.) and Iodomethane (6.67 g, 47.01 mmol, 2.93 mL, 2.0 equiv.). The mixture was stirred at 20° C. for 2 hours. The mixture was filtered, and the filter cake was washed by EtOAc (200 mL). The crude product was triturated with EtOAc at 25° C. for 20 minutes. The mixture was filtered, and organic layer was concentrated under reduced pressure to give 3-iodo-5-methoxy-1-methyl-6-nitro-indazole (6.8 g, crude).

LC-MS (ESI): m/z=334.1 [M+H]⁺

¹H NMR (400 MHz, DMSO-d₆) δ=8.41 (s, 1H), 7.10 (s, 1H), 4.09 (s, 3H), 3.96 (s, 3H)

### Step 3: 3-(2,6-dibenzyloxy-3-pyridyl)-5-methoxy-1-methyl-6-nitro-indazole

A mixture of 3-iodo-5-methoxy-1-methyl-6-nitro-indazole (6.8 g, 20.42 mmol, 1.0 equiv.), 2,6-dibenzyloxy-3-(4,4,5,5-tetramethyl-1,3,2-dioxaborolan-2-yl)pyridine (9.37 g, 22.46 mmol, 1.1 equiv.), cesium carbonate (19.96 g, 61.25 mmol, 3.0 equiv.), and Pd(dppf)Cl₂ (1.49 g, 2.04 mmol, 0.1 equiv.) in THE (2 mL) and H₂O (0.2 mL) was degassed and purged with N₂ three times. The mixture was stirred at 60° C. for 16 hours under a N₂ atmosphere. The reaction mixture was concentrated under reduced pressure to give a residue, which was purified by flash silica gel chromatography (0~20% ethyl acetate/petroleum ether) to give 3-(2,6-dibenzyloxy-3-pyridyl)-5-methoxy-1-methyl-6-nitro-indazole (4 g, crude) as a yellow solid.

LC-MS (ESI): m/z=497.3 [M+H]⁺

¹H NMR (400 MHz, DMSO-d₆) δ=8.34 (s, 1H), 7.95 (d, J=8.0 Hz, 1H), 7.48 (d, J=7.2 Hz, 2H), 7.43-7.39 (m, 2H), 7.39-7.34 (m, 5H), 7.32-7.27 (m, 3H), 6.63 (d, J=8.0 Hz, 1H), 5.52 (s, 2H), 5.45 (s, 2H), 4.12 (s, 3H), 3.56 (s, 3H)

### Step 4: 3-(6-amino-5-methoxy-1-methyl-indazol-3-yl)piperidine-2,6-dione

To a solution of 3-(2,6-dibenzyloxy-3-pyridyl)-5-methoxy-1-methyl-6-nitro-indazole (4.0 g, 8.06 mmol, 1.0 equiv.) in THE (40 mL) and EtOAc (40 mL) was added Pd on carbon (10 wt %, 1.71 g, 1.61 mmol, 0.2 equiv.) and Pd(OH)₂ on carbon (20 wt %, 1.13 g, 1.61 mmol, 0.2 equiv.) under a N₂ atmosphere. The suspension was degassed and purged with H₂ three times. The mixture was stirred under H₂ (50 psi) at 25° C. for 24 hours. The reaction mixture was filtered and concentrated under reduced pressure to give a residue, which was triturated with EtOAc at 25° C. for 30 min to give 3-(6-amino-5-methoxy-1-methyl-indazol-3-yl)piperidine-2,6-dione (976 mg, crude), which was used in the next step without further purification.

LC-MS (ESI): m/z=289.2 [M+H]⁺

### Step 5: tert-butyl 4-[3-(4-bromo-3-methyl-phenoxy)propyl]piperidine-1-carboxylate

To a solution of tert-butyl 4-(3-bromopropyl)piperidine-1-carboxylate (5 g, 16.33 mmol, 1.0 equiv.) in MeCN (50 mL) was added potassium carbonate (6.77 g, 48.98 mmol, 3.0 equiv.) and 4-bromo-3-methyl-phenol (3.05 g, 16.33 mmol, 1.0 equiv.). The mixture was stirred at 60° C. for 12 hours. The reaction mixture was filtered and concentrated under reduced pressure to give tert-butyl 4-[3-(4-bromo-3-methyl-phenoxy)propyl]piperidine-1-carboxylate (6.5 g) as a yellow oil, which was used in the next step without further purification.

LC-MS (ESI): m/z=312.1 [M+H]⁺

### Step 6: 4-[3-(4-bromo-3-methyl-phenoxy)propyl]piperidine

A solution of tert-butyl 4-[3-(4-bromo-3-methyl-phenoxy)propyl]piperidine-1-carboxylate (3.0 g, 7.28 mmol, 1.0 equiv.) in HCl/dioxane (50 mL) was stirred at 25° C. for 1.5 hours. The reaction mixture was concentrated under reduced pressure to give 4-[3-(4-bromo-3-methyl-phenoxy)propyl]piperidine (3 g, crude, HCl) as a white solid, which was used in the next step without further purification.

LC-MS (ESI): m/z=312.2 [M+H]⁺

¹H NMR (400 MHz, DMSO-d₆) δ=7.42 (d, J=8.8 Hz, 1H), 6.94 (s, 1H), 6.71 (dd, J=2.4, 8.0 Hz, 1H), 3.93 (t, J=6.4 Hz, 2H), 3.21 (d, J=11.6 Hz, 2H), 2.79 (q, J=12.0 Hz, 2H), 2.29 (s, 3H), 1.79 (d, J=13.2 Hz, 3H), 1.74-1.66 (m, 2H), 1.54 (d, J=7.2 Hz, 1H), 1.37-1.26 (m, 4H)

### Step 7: ethyl 2-[4-[3-(4-bromo-3-methyl-phenoxy)propyl]-1-piperidyl]acetate

To a solution of 4-[3-(4-bromo-3-methyl-phenoxy)propyl]piperidine (3 g, 9.61 mmol, 1.0 equiv.) in MeCN (60 mL) was added potassium carbonate (3.98 g, 28.82 mmol, 3.0 equiv.) and ethyl 2-bromoacetate (1.60 g, 9.61 mmol, 1.06 mL, 1.0 equiv.). The mixture was stirred at 60° C. for 12 hours. The reaction mixture was filtered and organic layer concentrated under reduced pressure to give a residue, which was purified by flash silica gel chromatography (0-20% ethyl acetate/petroleum ether) to give ethyl 2-[4-[3-(4-bromo-3-methyl-phenoxy)propyl]-1-piperidyl]acetate (2 g, crude) as a yellow solid.

LC-MS (ESI): m/z=398.3 [M+H]⁺

¹H NMR (400 MHz, DMSO-d₆) δ=7.43 (d, J=8.8 Hz, 1H), 6.95 (d, J=2.4 Hz, 1H), 6.71 (dd, J=8.8, 2.8 Hz, 1H), 4.07 (q, J=7.2 Hz, 2H), 3.92 (t, J=6.4 Hz, 2H), 3.30 (s, 2H), 3.16 (s, 2H), 2.80 (d, J=11.2 Hz, 2H), 2.30 (s, 3H), 2.11 (t, J=10.4 Hz, 2H), 1.74-1.66 (m, 2H), 1.62 (d, J=11.6 Hz, 2H), 1.37-1.28 (m, 2H), 1.19 (t, J=7.2 Hz, 3H), 1.15-1.11 (m, 1H)

### Step 8: tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-piperidyl]propoxy]-2-methyl-phenyl]pyridine-2-carboxylate

Tert-butyl 6-[8-(1,3-benzothiazol-2-ylcarbamoyl)-3,4-dihydro-]H-isoquinolin-2-yl]-3-(4,4,5,5-tetramethyl-1,3,2-dioxaborolan-2-yl)pyridine-2-carboxylate (922.66 mg, 1.51 mmol, 1.2 equiv.), ethyl 2-[4-[3-(4-bromo-3-methyl-phenoxy)propyl]-1-piperidyl]acetate (500 mg, 1.26 mmol, 1.0 equiv.), and potassium carbonate (520.44 mg, 3.77 mmol, 3.0 equiv.) were taken up into a Schlenk tube in dioxane (5 mL) followed by the addition of Ad₂nBup Pd G₃ (91.41 mg, 125.52 mol, 0.1 equiv.). The Schlenk tube was heated at 100° C. for 1.5 hours. The reaction mixture was concentrated under reduced pressure to give a residue, which was purified by flash silica gel chromatography (0-20% ethyl acetate/petroleum ether) to give tert-butyl 6-[8-(1,3-benzothiazol-2-ylcarbamoyl)-3,4-dihydro-]H-isoquinolin-2-yl]-3-[4-[3-[1-(2-ethoxy-2-oxo-ethyl)-4-piperidyl]propoxy]-2-methyl-phenyl]pyridine-2-carboxylate (450 mg, crude) as a brown solid.

LC-MS (ESI): m/z=804.7 [M+H]⁺

### Step 9: 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid

To a solution of tert-butyl 6-[8-(1,3-benzothiazol-2-ylcarbamoyl)-3,4-dihydro-]H-isoquinolin-2-yl]-3-[4-[3-[1-(2-ethoxy-2-oxo-ethyl)-4-piperidyl]propoxy]-2-methyl-phenyl]pyridine-2-carboxylate (450 mg, 559.70 mol, 1.0 equiv.) in THE (16 mL) and H₂O (8 mL) was added LiOH·H₂O (117.43 mg, 2.80 mmol, 5 equiv.). The mixture was stirred at 25° C. for 3 hours. The reaction mixture was concentrated under reduced pressure to remove THF. The reaction mixture was quenched by the addition of H₂O (5 mL) at 25° C. and extracted with DCM (10 mL). The combined organic layers were dried over sodium sulfate, filtered, and concentrated under reduced pressure to give 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid (400 mg, crude) as a yellow solid, which was used in the next step without further purification.

LC-MS (ESI): m/z=776.6 [M+H]⁺

### Step 10: tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylate

To a solution of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid (100 mg, 128.87 mol, 1.0 equiv.) and 3-(6-amino-5-methoxy-1-methyl-indazol-3-yl)piperidine-2,6-dione (1.1 equiv.) in DMF (2 mL) was added HATU (73.50 mg, 193.31 mol, 1.5 equiv.) and DIPEA (49.97 mg, 386.62 mol, 67.34 L, 3.0 equiv.). The mixture was stirred at 40° C. for 2 hours. The reaction mixture was adjusted to pH=4 by 1 M HCl solution then diluted with H₂O (2 mL) and extracted with ethyl acetate (6 mL). The combined organic layers were concentrated under reduced pressure to give tert-butyl 6-[8-(1,3-benzothiazol-2-ylcarbamoyl)-3,4-dihydro-]H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylate (80 mg, crude), which was used in the next step without further purification.

LC-MS (ESI): m/z=790.7 [M+H]⁺

### Step 11: 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-]H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylate (100 mg, 95.58 mol, 1.0 equiv.) in TFA (1 mL) was added DCM (1 mL). The mixture was stirred at 25° C. for 16 hours. The reaction mixture was concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-5-methoxy-1-methyl-indazol-6-yl]amino]-2-oxo-ethyl]-4-piperidyl]propoxy]-2-methyl-phenyl]pyridine-2-carboxylic acid as a white solid.

LC-MS (ESI): m/z=990.7 [M+H]⁺

¹H NMR (400 MHz, DMSO-d₆) δ=12.95-12.46 (m, 2H), 10.86 (s, 1H), 10.13 (s, 1H), 8.39 (s, 1H), 8.13 (s, 1H), 8.03 (d, J=7.6 Hz, 1H), 7.79 (d, J=8.0 Hz, 1H), 7.62 (d, J=7.6 Hz, 1H), 7.49-7.43 (m, 3H), 7.40-7.32 (m, 2H), 7.28 (s, 1H), 6.94 (dd, J=8.8, 14.4 Hz, 2H), 6.80 (d, J=2.4 Hz, 1H), 6.72 (dd, J=2.4, 8.4 Hz, 1H), 4.97 (s, 2H), 4.33 (dd, J=5.2, 9.6 Hz, 1H), 3.96 (t, J=6.4 Hz, 2H), 3.92 (d, J=6.0 Hz, 8H), 3.30 (s, 2H), 3.02 (t, J=5.6 Hz, 2H), 2.92-2.82 (m, 2H), 2.65-2.59 (m, 2H), 2.40-2.34 (m, 1H), 2.27-2.11 (m, 3H), 2.03 (s, 3H), 1.77 (d, J=8.8 Hz, 4H), 1.44-1.38 (m, 2H), 1.35-1.21 (m, 3H)

### Example 20: 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid (Compound 105)

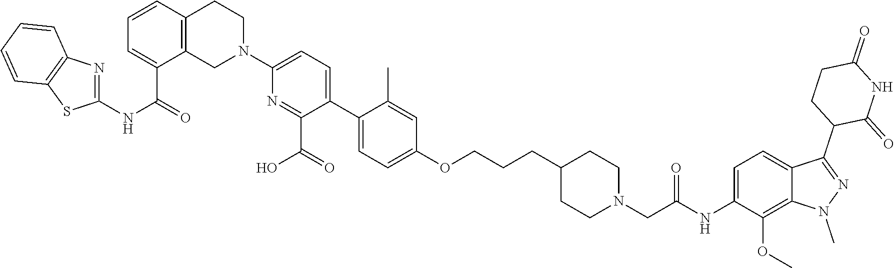 [SMILES: COc1c(NC(=O)CN2CCC(CCCOc3ccc(-c4ccc(N5CCc6cccc(C(=O)Nc7nc8ccccc8s7)c6C5)nc4C(=O)O)c(C)c3)CC2)ccc2c(C3CCC(=O)NC3=O)nn(C)c12]

### Step 1: tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate

To a solution of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid (100 mg, 128.87 mol, 1 equiv.) and 3-(6-amino-7-methoxy-1-methyl-indazol-3-yl)piperidine-2,6-dione (41.85 mg, 128.87 mol, 1 equiv., HCl) in pyridine (1 mL) was added EDCI (37.06 mg, 193.31 mol, 1.5 equiv.). The mixture was stirred at 60° C. for 2 hours. The reaction mixture was diluted with water (10 mL) and extracted with EtOAc (5 mL). The combined organic layers were washed with brine (10 mL), filtered, and concentrated under reduced pressure to give the crude product tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate (100 mg) as a yellow solid.

LC-MS (ESI): m/z=523.8 [M+H]⁺

### Step 2: 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylate (100 mg, 95.58 mol, 1 equiv.) in TFA (1 mL) and DCM (1 mL) was stirred at 25° C. for 16 hours. The reaction mixture was concentrated under reduced pressure to remove solvent to give a residue, which was purified by prep-HPLC to give 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-methyl-phenyl]pyridine-2-carboxylic acid (37.85 mg) as a white solid.

LC-MS (ESI): m/z=495.7 [M+H]⁺

¹H NMR (400 MHz, DMSO-d₆) δ=10.90 (s, 1H), 9.85 (s, 1H), 8.16 (s, 1H), 8.02 (dd, J=8.4, 11.6 Hz, 2H), 7.80 (d, J=8.0 Hz, 1H), 7.63 (d, J=7.2 Hz, 1H), 7.50-7.41 (m, 4H), 7.41-7.33 (m, 2H), 6.94 (dd, J=6.4, 8.4 Hz, 2H), 6.80 (d, J=2.4 Hz, 1H), 6.72 (dd, J=2.4, 8.4 Hz, 1H), 4.98 (s, 2H), 4.34 (dd, J=5.2, 10.0 Hz, 1H), 4.15 (s, 3H), 3.96 (t, J=6.4 Hz, 2H), 3.93-3.88 (m, 5H), 3.17 (s, 2H), 3.03 (t, J=5.6 Hz, 2H), 2.95 (d, J=10.8 Hz, 2H), 2.69-2.61 (m, 2H), 2.39-2.32 (m, 1H), 2.26 (t, J=10.8 Hz, 2H), 2.20-2.14 (m, 1H), 2.04 (s, 3H), 1.82-1.72 (m, 4H), 1.46-1.38 (m, 2H), 1.37-1.25 (m, 3H)

### Example 23: 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid (Compound 114)

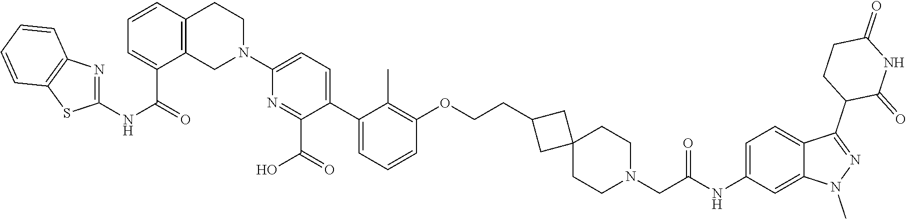 [SMILES: Cc1c(OCCC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### Step 1: tert-butyl 2-(2-ethoxy-2-oxoethylidene)-7-azaspiro[3.5]nonane-7-carboxylate

A mixture of NaH (250.70 mg, 6.27 mmol, 60% purity, 1.5 equiv.) in THE (20 mL) was degassed and purged with N₂ three times. To this mixture was added dropwise ethyl 2-(diethoxyphosphoryl)acetate (1.31 g, 5.85 mmol, 1.16 mL, 1.4 equiv.) at 0° C. under a N₂ atmosphere. The mixture was stirred at 0° C. for 1 hour followed by the addition of tert-butyl 2-oxo-7-azaspiro[3.5]nonane-7-carboxylate (1.0 g, 4.18 mmol, 1.0 equiv.) in THE (5 mL). The resulting mixture was stirred at 25° C. for 1 hour. After completion, the solution was cooled to 0° C. and quenched with saturated ammonium chloride solution (100 mL). The solution was extracted with ethyl acetate (150 mL). The combined organic layers were washed with brine (200 mL), dried over anhydrous sodium sulfate, filtered, and concentrated under reduced pressure to give tert-butyl 2-(2-ethoxy-2-oxoethylidene)-7-azaspiro[3.5]nonane-7-carboxylate (1.2 g) as a colorless oil, which was used in the next step without further purification.

LC-MS (ESI): m/z=254.0 [M+H−56]⁺.

### Step 2: tert-butyl 2-(2-ethoxy-2-oxoethyl)-7-azaspiro[3.5]nonane-7-carboxylate

A mixture of tert-butyl 2-(2-ethoxy-2-oxoethylidene)-7-azaspiro[3.5]nonane-7-carboxylate (1.2 g, 3.88 mmol, 1.0 equiv.), Pd on carbon (10 wt %, 825.50 mg, 775.70 mol, 0.2 equiv.), THE (15 mL), and Pd(OH)₂ on carbon (20 wt %, 544.67 mg, 775.70 mol, 0.2 equiv.) in EtOH (15 mL) was stirred at 25° C. for 12 hours under H₂ (15 psi). The mixture was filtered, washed with THE (100 mL), and the filter liquid was concentrated under reduced pressure to give tert-butyl 2-(2-ethoxy-2-oxoethyl)-7-azaspiro[3.5]nonane-7-carboxylate (1.2 g) as a colorless oil, which was used in the next step without further purification.

LC-MS (ESI): m/z=256.1 [M+H−56]⁺.

### Step 3: tert-butyl 2-(2-hydroxyethyl)-7-azaspiro[3.5]nonane-7-carboxylate

To a solution of tert-butyl 2-(2-ethoxy-2-oxoethyl)-7-azaspiro[3.5]nonane-7-carboxylate (1.20 g, 3.85 mmol, 1.0 equiv.) in THF (15 mL) was added LAH (2.5 M, 1.85 mL, 1.2 equiv.). The mixture was stirred at 0° C. for 1 hour. The reaction mixture was quenched by the addition of water (1 mL) and 15% NaOH (1 mL) in water (10 mL). The reaction mixture was mixed with sodium sulfate, filtered, and concentrated under reduced pressure to give a residue, which was purified by flash silica gel chromatography (0~12% ethyl acetate/petroleum ether) to give tert-butyl 2-(2-hydroxyethyl)-7-azaspiro[3.5]nonane-7-carboxylate (880 mg) as a colorless oil.

LC-MS (ESI): m/z=214.3 [M+H−56]+.

¹H NMR (400 MHz, DMSO-d₆) δ=4.27 (t, J=5.2 Hz, 1H), 3.31-3.28 (m, 2H), 3.27-3.22 (m, 2H), 3.18-3.13 (m, 2H), 2.27 (quin, J=8.0 Hz, 1H), 1.94-1.84 (m, 2H), 1.52 (q, J=7.2 Hz, 2H), 1.49-1.44 (m, 2H), 1.37 (s, 9H), 1.36-1.30 (m, 4H).

### Step 4: tert-butyl 2-(2-(3-bromo-2-methylphenoxy)ethyl)-7-azaspiro[3.5]nonane-7-carboxylate

A mixture of tert-butyl 2-(2-hydroxyethyl)-7-azaspiro[3.5]nonane-7-carboxylate (880 mg, 3.27 mmol, 1.0 equiv.), 3-bromo-2-methylphenol (733.20 mg, 3.92 mmol, 1.2 equiv.), and 2-(tributyl-phosphanylidene)acetonitrile (946.14 mg, 3.92 mmol, 1.2 equiv.) in toluene (10 mL) was degassed and purged with N₂ three times. The mixture was stirred at 120° C. for 12 hours under a N₂ atmosphere. The reaction mixture was concentrated under reduced pressure to remove solvent. The residue was purified by column chromatography (SiO₂, petroleum ether/ethyl acetate=20/1 to 0/1) to give tert-butyl 2-(2-(3-bromo-2-methylphenoxy)ethyl)-7-azaspiro[3.5]nonane-7-carboxylate (1 g) as a yellow oil.

LC-MS (ESI): m/z=382.0 [M+H−56]⁺.

### Step 5: 2-(2-(3-bromo-2-methylphenoxy)ethyl)-7-azaspiro[3.5]nonane

To a solution of tert-butyl 2-(2-(3-bromo-2-methylphenoxy)ethyl)-7-azaspiro[3.5]nonane-7-carboxylate (1 g, 2.28 mmol, 1.0 equiv.) in DCM (10 mL) was added HCl/dioxane (4 M, 570.26 μL, 1.0 equiv.). The mixture was stirred at 25° C. for 1 hour. The reaction mixture was filtered and concentrated under reduced pressure to give a residue, which was triturated with petroleum ether:ethyl acetate=3:1 at 25° C. for 15 mins to give 2-(2-(3-bromo-2-methylphenoxy)ethyl)-7-azaspiro[3.5]nonane (500 mg) as a white solid.

LC-MS (ESI): m/z=338.0 [M+H]⁺

¹H NMR (400 MHz, DMSO-d₆) δ=7.19-7.04 (m, 2H), 6.94 (d, J=8.0 Hz, 1H), 3.91 (t, J=6.0 Hz, 2H), 3.54 (t, J=6.8 Hz, 1H), 2.96 (s, 2H), 2.87 (s, 2H), 2.39 (dd, J=7.6, 15.6 Hz, 1H), 2.22 (s, 3H), 1.98 (d, J=8.8 Hz, 2H), 1.87-1.83 (m, 2H), 1.74 (s, 2H), 1.66-1.62 (m, 2H), 1.54-1.47 (m, 2H)

### Step 6: ethyl 2-(2-(2-(3-bromo-2-methylphenoxy)ethyl)-7-azaspiro[3.5]nonan-7-yl)acetate

To a solution of 2-(2-(3-bromo-2-methylphenoxy)ethyl)-7-azaspiro[3.5]nonane (500 mg, 1.48 mmol, 1.0 equiv.) and potassium carbonate (612.83 mg, 4.43 mmol, 3.0 equiv.) in DMF (5 mL), was added ethyl 2-bromoacetate (246.84 mg, 1.48 mmol, 163.58 μL, 1.0 equiv.). The mixture was stirred at 40° C. for 1 hour. The reaction mixture was concentrated under reduced pressure to remove solvent. The residue was diluted with H₂O (50 mL) and extracted with ethyl acetate (100 mL). The combined organic layers were washed with H₂O (500 mL), dried over sodium sulfate, filtered, and concentrated under reduced pressure to give ethyl 2-(2-(2-(3-bromo-2-methylphenoxy)ethyl)-7-azaspiro[3.5]nonan-7-yl)acetate (550 mg) as a yellow oil, which was used in the next step without further purification.

LC-MS (ESI): m/z=424.1 [M+H]⁺

### Step 7: tert-butyl 6-(8-(benzo[d]thiazol-2-ylcarbamoyl)-3,4-dihydroisoquinolin-2(1H)-yl)-3-(3-(2-(7-(2-ethoxy-2-oxoethyl)-7-azaspiro[3.5]nonan-2-yl)ethoxy)-2-methylphenyl)picolinate

A mixture of ethyl 2-(2-(2-(3-bromo-2-methylphenoxy)ethyl)-7-azaspiro[3.5]nonan-7-yl)acetate (300 mg, 706.93 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (476.33 mg, 777.62 mol, 1.1 equiv.), KF (123.21 mg, 2.12 mmol, 49.68 μL, 3.0 equiv.), Ad₂nBup Pd G₃ (51.48 mg, 70.69 mol, 0.1 equiv.), and H₂O (0.6 mL) in dioxane (6 mL) was degassed and purged with N₂ three times. The mixture was stirred at 100° C. for 1 hour under a N₂ atmosphere. The reaction mixture was concentrated under reduced pressure to remove solvent. The residue was purified by column chromatography (SiO₂, petroleum ether/ethyl acetate=10/1 to 3/2) to give tert-butyl 6-(8-(benzo[d]thiazol-2-ylcarbamoyl)-3,4-dihydroisoquinolin-2(1H)-yl)-3-(3-(2-(7-(2-ethoxy-2-oxoethyl)-7-azaspiro[3.5]nonan-2-yl)ethoxy)-2-methylphenyl)picolinate (350 mg) as a yellow solid.

LC-MS (ESI): m/z=830.2 [M+H]⁺

¹H NMR (400 MHz, DMSO-d₆) δ=12.87 (s, 1H), 8.02 (d, J=7.6 Hz, 1H), 7.78 (d, J=8.0 Hz, 1H), 7.60 (d, J=7.6 Hz, 1H), 7.44 (dd, J=4.0, 8.4 Hz, 2H), 7.40-7.31 (m, 3H), 7.12-7.06 (m, 1H), 6.96-6.87 (m, 2H), 6.57 (s, 1H), 4.97 (s, 2H), 4.05 (q, J=7.2 Hz, 2H), 3.94-3.86 (m, 4H), 3.11 (s, 2H), 3.03 (t, J=5.6 Hz, 2H), 2.41-2.29 (m, 6H), 1.86 (s, 3H), 1.82 (s, 1H), 1.52 (s, 2H), 1.46-1.39 (m, 4H), 1.16 (t, J=7.2 Hz, 3H), 1.07 (s, 2H), 1.00 (s, 9H).

### Step 8: 2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-7-azaspiro[3.5]nonan-7-yl)acetic acid

To a solution of tert-butyl 6-(8-(benzo[d]thiazol-2-ylcarbamoyl)-3,4-dihydroisoquinolin-2(1H)-yl)-3-(3-(2-(7-(2-ethoxy-2-oxoethyl)-7-azaspiro[3.5]nonan-2-yl)ethoxy)-2-methylphenyl)picolinate (350 mg, 421.66 mol, 1.0 equiv.) in THE (5 mL) was added LiOH·H₂O (53.08 mg, 1.26 mmol, 3.0 equiv.) and H₂O (1 mL). The mixture was stirred at 60° C. for 1 hour. The reaction mixture was acidified to pH=5-6 with 1 M HCl. The mixture was filtered and concentrated under reduced pressure to give 2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-7-azaspiro[3.5]nonan-7-yl)acetic acid (280 mg) as a yellow solid, which was used in the next step without further purification.

LC-MS (ESI): m/z=802.8 [M+H]⁺

### Step 9: tert-butyl 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate

To a solution of 2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-7-azaspiro[3.5]nonan-7-yl)acetic acid (280 mg, 349.13 mol, 1.0 equiv.) in pyridine (3 mL) was added EDCI (100.39 mg, 523.70 mol, 1.5 equiv.) and 3-(6-amino-1-methyl-indazol-3-yl)piperidine-2,6-dione (108.21 mg, 418.96 mol, 1.2 equiv.). The mixture was stirred at 60° C. for 1 hour. The reaction mixture was concentrated under reduced pressure to remove solvent. The residue was diluted with H₂O (50 mL), extracted with DCM (150 mL), washed with brine (50 mL), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate (300 mg) as a yellow solid, which was used in the next step without further purification.

LC-MS (ESI): m/z=1042.5 [M+H]⁺

### Step 10: 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate (300 mg, 287.84 mol, 1.0 equiv.) in TFA (4 mL) was added DCM (1 mL). The mixture was stirred at 40° C. for 1 hour. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid (160.33 mg) as a yellow solid.

LC-MS (ESI): m/z=986.3 [M+H]⁺

¹H NMR (400 MHz, DMSO-d₆) δ=12.86 (dt, J=6.0, 9.0 Hz, 1H), 12.73-12.35 (m, 1H), 10.89 (s, 1H), 8.06-7.97 (m, 2H), 7.79 (d, J=8.0 Hz, 1H), 7.67-7.60 (m, 2H), 7.49-7.43 (m, 3H), 7.39-7.32 (m, 2H), 7.18 (d, J=8.4 Hz, 1H), 7.12-7.06 (m, 1H), 6.97 (d, J=8.8 Hz, 1H), 6.86 (d, J=8.4 Hz, 1H), 6.62 (d, J=7.6 Hz, 1H), 4.98 (s, 2H), 4.33 (dd, J=5.2, 9.6 Hz, 1H), 3.92 (s, 3H), 3.90 (s, 4H), 3.03 (t, J=5.6 Hz, 2H), 2.71-2.56 (m, 4H), 2.49-2.29 (m, 6H), 2.21-2.14 (m, 1H), 1.99 (s, 2H), 1.89 (s, 3H), 1.88-1.83 (m, 2H), 1.74 (d, J=6.0 Hz, 2H), 1.67 (dd, J=0.8, 17.2 Hz, 2H), 1.50 (t, J=9.6 Hz, 2H)

### Example 24: 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid (Compound 115)

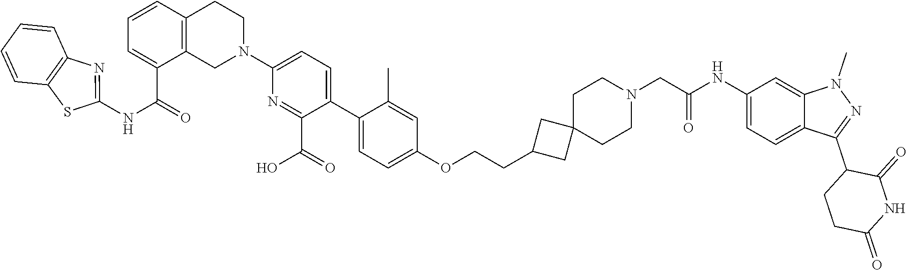 [SMILES: Cc1cc(OCCC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O]

### Step 1: tert-butyl 2-(2-(4-bromo-3-methylphenoxy)ethyl)-7-azaspiro[3.5]nonane-7-carboxylate

A mixture of 4-bromo-3-methylphenol (833.18 mg, 4.45 mmol, 1.2 equiv.), tert-butyl 2-(2-hydroxyethyl)-7-azaspiro[3.5]nonane-7-carboxylate (1.0 g, 3.71 mmol, 1.0 equiv.), and 2-(tributyl-phosphanylidene)acetonitrile (1.08 g, 4.45 mmol, 1.2 equiv.) in toluene (10 mL) was degassed and purged with N₂ three times. The mixture was stirred at 120° C. for 12 hours under a N₂ atmosphere. The reaction mixture was concentrated under reduced pressure to remove solvent. The residue was purified by column chromatography (SiO₂, petroleum ether/ethyl acetate=20/1 to 0/1) to give tert-butyl 2-(2-(4-bromo-3-methylphenoxy)ethyl)-7-azaspiro[3.5]nonane-7-carboxylate (1.5 g) as a yellow oil.

LC-MS (ESI): m/z=382.0 [M+H−56]⁺.

### Step 2: 2-(2-(4-bromo-3-methylphenoxy)ethyl)-7-azaspiro[3.5]nonane

To a solution of tert-butyl 2-(2-(4-bromo-3-methylphenoxy)ethyl)-7-azaspiro[3.5]nonane-7-carboxylate (1.5 g, 3.42 mmol, 1.0 equiv.) in DCM (15 mL) was added HCl/dioxane (4 M, 855.39 μL, 1.0 equiv.). The mixture was stirred at 25° C. for 1 hour. The reaction mixture was filtered and concentrated under reduced pressure to give a residue, which was triturated with petroleum ether:ethyl acetate=3:1 at 25° C. for 15 mins to give 2-(2-(4-bromo-3-methylphenoxy)ethyl)-7-azaspiro[3.5]nonane (860 mg) as a white solid.

LC-MS (ESI): m/z=338.1 [M+H]⁺

¹H NMR (400 MHz, DMSO-d₆) δ=7.19-7.03 (m, 2H), 6.94 (d, J=8.4 Hz, 1H), 3.92 (s, 2H), 3.54 (t, J=6.8 Hz, 1H), 2.96 (d, J=4.8 Hz, 2H), 2.88 (s, 2H), 2.43-2.37 (m, 1H), 2.22 (s, 3H), 2.02-1.96 (m, 2H), 1.89-1.82 (m, 2H), 1.73 (d, J=5.6 Hz, 2H), 1.66-1.62 (m, 2H), 1.55-1.47 (m, 2H)

### Step 3: ethyl 2-(2-(2-(4-bromo-3-methylphenoxy)ethyl)-7-azaspiro[3.5]nonan-7-yl)acetate

To a solution of 2-(2-(4-bromo-3-methylphenoxy)ethyl)-7-azaspiro[3.5]nonane (860 mg, 2.54 mmol, 1.0 equiv.) and potassium carbonate (1.05 g, 7.63 mmol, 3.0 equiv.) in DMF (10 mL), was added ethyl 2-bromoacetate (424.56 mg, 2.54 mmol, 281.35 μL, 1.0 equiv.). The mixture was stirred at 40° C. for 1 hour. The reaction mixture was concentrated under reduced pressure to remove solvent. The residue was diluted with H₂O (50 mL) and extracted with ethyl acetate (100 mL). The combined organic layers were washed with H₂O (500 mL), dried over sodium sulfate, filtered, and concentrated under reduced pressure to give ethyl 2-(2-(2-(4-bromo-3-methylphenoxy)ethyl)-7-azaspiro[3.5]nonan-7-yl)acetate (1.0 g) as a yellow oil, which was used into next step without further purification.

LC-MS (ESI): m/z=424.1 [M+H]⁺

### Step 4: tert-butyl 6-(8-(benzo[d]thiazol-2-ylcarbamoyl)-3,4-dihydroisoquinolin-2(1H)-yl)-3-(4-(2-(7-(2-ethoxy-2-oxoethyl)-7-azaspiro[3.5]nonan-2-yl)ethoxy)-2-methylphenyl)picolinate

A mixture of ethyl 2-(2-(2-(4-bromo-3-methylphenoxy)ethyl)-7-azaspiro[3.5]nonan-7-yl)acetate (300 mg, 706.93 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (476.33 mg, 777.62 mol, 1.1 equiv.), KF (123.21 mg, 2.12 mmol, 49.68 μL, 3.0 equiv.), Ad₂nBup Pd G₃ (51.48 mg, 70.69 μmol, 0.1 equiv.), and H₂O (0.3 mL) in dioxane (3 mL) was degassed and purged with N₂ three times. The mixture was stirred at 100° C. for 1 hour under a N₂ atmosphere. The reaction mixture was concentrated under reduced pressure to remove solvent. The residue was purified by column chromatography (SiO₂, petroleum ether/ethyl acetate=10/1 to 3/2) to give tert-butyl 6-(8-(benzo[d]thiazol-2-ylcarbamoyl)-3,4-dihydroisoquinolin-2(1H)-yl)-3-(4-(2-(7-(2-ethoxy-2-oxoethyl)-7-azaspiro[3.5]nonan-2-yl)ethoxy)-2-methylphenyl)picolinate (300 mg) as a yellow solid.

LC-MS (ESI): m/z=830.2 [M+H]⁺

¹H NMR (400 MHz, DMSO-d₆) δ=12.86 (s, 1H), 8.01 (d, J=8.0 Hz, 1H), 7.77 (d, J=8.0 Hz, 1H), 7.60 (d, J=7.6 Hz, 1H), 7.45-7.42 (m, 2H), 7.35 (td, J=7.2, 14.3 Hz, 3H), 7.11-7.06 (m, 1H), 6.95-6.87 (m, 2H), 6.56 (d, J=7.2 Hz, 1H), 4.97 (s, 2H), 4.04 (q, J=7.2 Hz, 2H), 3.91-3.85 (m, 4H), 3.10 (s, 2H), 3.03 (t, J=5.6 Hz, 2H), 2.41-2.26 (m, 7H), 1.85 (s, 3H), 1.83-1.79 (m, 2H), 1.51 (t, J=4.8 Hz, 2H), 1.45-1.38 (m, 4H), 1.16 (t, J=7.2 Hz, 3H), 1.00 (s, 9H)

### Step 5: 2-(2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)ethyl)-7-azaspiro[3.5]nonan-7-yl)acetic acid

To a solution of tert-butyl 6-(8-(benzo[d]thiazol-2-ylcarbamoyl)-3,4-dihydroisoquinolin-2(1H)-yl)-3-(4-(2-(7-(2-ethoxy-2-oxoethyl)-7-azaspiro[3.5]nonan-2-yl)ethoxy)-2-methylphenyl)picolinate (300 mg, 361.43 mol, 1.0 equiv.) in THE (3 mL) was added LiOH·H₂O (45.50 mg, 1.08 mmol, 3.0 equiv.) and H₂O (1 mL). The mixture was stirred at 60° C. for 1 hour. The reaction mixture was acidified to pH=5-6 with 1 M HCl. The mixture was filtered and concentrated under reduced pressure to give 2-(2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)ethyl)-7-azaspiro[3.5]nonan-7-yl)acetic acid (280 mg) as a yellow solid, which was used into next step without further purification.

LC-MS (ESI): m/z=802.4 [M+H]⁺

### Step 6: tert-butyl 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate

To a solution of 2-(2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)ethyl)-7-azaspiro[3.5]nonan-7-yl)acetic acid (280 mg, 349.13 mol, 1.0 equiv.) in pyridine (3 mL) was added EDCI (100.39 mg, 523.70 mol, 1.5 equiv.) and 3-(6-amino-1-methyl-indazol-3-yl)piperidine-2,6-dione (108.21 mg, 418.96 mol, 1.2 equiv.). The mixture was stirred at 60° C. for 1 hour. The reaction mixture was concentrated under reduced pressure to remove solvent. The residue was diluted with H₂O (50 mL), extracted with DCM (150 mL), washed with DCM (50 mL), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate (300 mg) as a yellow solid, which was used in the next step without further purification.

LC-MS (ESI): m/z=1042.5 [M+H]⁺

### Step 7: 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinate (300 mg, 287.84 mol, 1.0 equiv.) in TFA (4 mL) was added DCM (1 mL). The mixture was stirred at 40° C. for 1 hour. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(4-(2-(7-(2-((3-(2,6-dioxopiperidin-3-yl)-1-methyl-1H-indazol-6-yl)amino)-2-oxoethyl)-7-azaspiro[3.5]nonan-2-yl)ethoxy)-2-methylphenyl)picolinic acid (124.27 mg) as a white solid.

LC-MS (ESI): m/z=986.3 [M+H]⁺

¹H NMR (400 MHz, DMSO-d₆) δ=12.09-11.72 (m, 1H), 10.05 (s, 1H), 9.00 (s, 1H), 7.32 (s, 1H), 7.24-7.16 (m, 2H), 6.96 (d, J=8.0 Hz, 1H), 6.80 (d, J=8.4 Hz, 2H), 6.68-6.59 (m, 3H), 6.58-6.49 (m, 2H), 6.39 (dd, J=1.6, 8.8 Hz, 1H), 6.29-6.22 (m, 1H), 6.14 (d, J=8.8 Hz, 1H), 6.03 (d, J=8.4 Hz, 1H), 5.80 (d, J=7.6 Hz, 1H), 4.15 (s, 2H), 3.50 (dd, J=5.2, 9.6 Hz, 1H), 3.09 (s, 3H), 3.08-3.05 (m, 2H), 2.27 (s, 2H), 2.20 (t, J=5.6 Hz, 2H), 1.85-1.74 (m, 2H), 1.67-1.46 (m, 8H), 1.39-1.30 (m, 1H), 1.13 (t, J=10.0 Hz, 2H), 1.06 (s, 3H), 1.05-1.00 (m, 2H), 0.83 (t, J=4.8 Hz, 2H), 0.74 (t, J=4.8 Hz, 2H), 0.64 (dd, J=8.8, 11.2 Hz, 2H)

### Example 25: 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid (Compound 116)

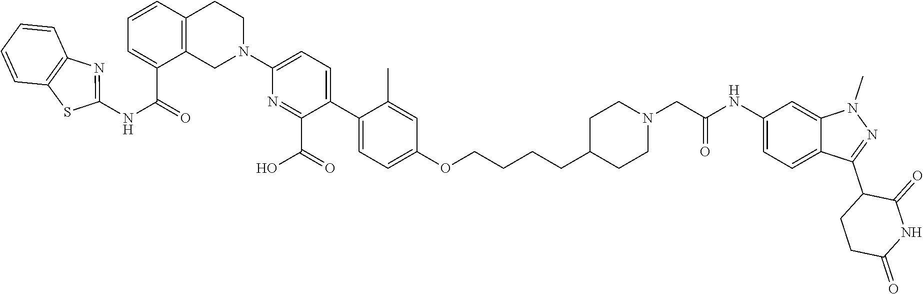 [SMILES: Cc1cc(OCCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: tert-butyl 4-[4-(4-bromo-3-methyl-phenoxy)butyl]piperidine-1-carboxylate

A mixture of tert-butyl 4-(4-hydroxybutyl)piperidine-1-carboxylate (2.0 g, 7.77 mmol, 1.0 equiv.), 4-bromo-3-methyl-phenol (1.74 g, 9.33 mmol, 1.2 equiv.), and 2-(tributyl-phosphanylidene)acetonitrile (2.81 g, 11.66 mmol, 1.5 equiv.) in toluene (30 mL) was degassed and purged with N₂ three times. The mixture was stirred at 120° C. for 24 hours under a N₂ atmosphere. The reaction mixture was concentrated under reduced pressure to give a residue, which was purified by flash silica gel chromatography (0-5% ethyl acetate/petroleum ether) to give tert-butyl 4-[4-(4-bromo-3-methyl-phenoxy)butyl]piperidine-1-carboxylate (3.2 g) as a brown oil.

LC-MS (ESI): m/z=326.1 [M+H−100]⁺

### Step 2: 4-[4-(4-bromo-3-methyl-phenoxy)butyl]piperidine

To a solution of tert-butyl 4-[4-(4-bromo-3-methyl-phenoxy)butyl]piperidine-1-carboxylate (3.2 g, 4.13 mmol, 1.0 equiv.) in dioxane (2 mL) was added HCl/dioxane (15 mL). The mixture was stirred at 25° C. for 0.5 hour. The reaction mixture was concentrated under reduced pressure to remove solvent. The crude product was triturated with (petroleum ether:ethyl acetate=3/1) at 25° C. for 10 mins to give 4-[4-(4-bromo-3-methyl-phenoxy)butyl]piperidine (1.4 g) as a white solid.

LC-MS (ESI): m/z=326.1 [M+H]⁺

### Step 3: ethyl 2-[4-[4-(4-bromo-3-methyl-phenoxy)butyl]-1-piperidyl]acetate

To a solution of 4-[4-(4-bromo-3-methyl-phenoxy)butyl]piperidine (400 mg, 1.10 mmol, 1.0 equiv., HCl) and potassium carbonate (457.22 mg, 3.31 mmol, 3.0 equiv.) in DMF (4 mL) was added ethyl 2-bromoacetate (184.16 mg, 1.10 mmol, 122.04 μL, 1.0 equiv.). The mixture was stirred at 60° C. for 1 hour. The reaction mixture was concentrated under reduced pressure to give a residue, which was purified by flash silica gel chromatography (0-13% ethyl acetate/petroleum ether) to give ethyl 2-[4-[4-(4-bromo-3-methyl-phenoxy)butyl]-1-piperidyl]acetate (400 mg) as a colorless oil.

LC-MS (ESI): m/z=412.1 [M+H]⁺

¹H NMR (400 MHz, DMSO-d₆) δ=7.42 (d, J=8.8 Hz, 1H), 6.94 (d, J=2.8 Hz, 1H), 6.73-6.68 (dd, J=8.8, 2.8 Hz, 1H), 3.93 (t, J=6.4 Hz, 2H), 3.62 (t, J=6.4 Hz, 1H), 3.14 (s, 2H), 2.78 (d, J=11.2 Hz, 2H), 2.29 (s, 3H), 1.70-1.56 (m, 6H), 1.44-1.34 (m, 3H), 1.24 (d, J=7.6 Hz, 2H), 1.18 (s, 3H), 1.15-1.07 (m, 3H)

### Step 4: tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butoxy]-2-methyl-phenyl]pyridine-2-carboxylate

A mixture of ethyl 2-[4-[4-(4-bromo-3-methyl-phenoxy)butyl]-1-piperidyl]acetate (200 mg, 485.01 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (356.51 mg, 582.02 mol, 1.2 equiv.), KF (84.53 mg, 1.46 mmol, 34.09 μL, 3.0 equiv.), H₂O (0.4 mL), and Ad₂nBup Pd G₃ (35.32 mg, 48.50 mol, 0.1 equiv.) in dioxane (4 mL) was degassed and purged with N₂ three times. The mixture was stirred at 100° C. for 1 hour under a N₂ atmosphere. The reaction mixture was concentrated under reduced pressure to give a residue, which was purified by flash silica gel chromatography (0~45% ethyl acetate/petroleum ether) to give tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butoxy]-2-methyl-phenyl]pyridine-2-carboxylate (300 mg) as a yellow solid.

LC-MS (ESI): m/z=818.4 [M+H]⁺

### Step 5: 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butoxy]-2-methyl-phenyl]pyridine-2-carboxylate (300 mg, 337.39 mol, 1.0 equiv.) in THF (2 mL) was added LiOH·H₂O (42.47 mg, 1.01 mmol, 3.0 equiv.) and H₂O (0.4 mL). The mixture was stirred at 40° C. for 1 hour. The reaction mixture was concentrated under reduced pressure to give a residue. The residue was diluted with H₂O (3 mL). The pH of mixture was adjusted to pH=4 with 1M HCl and filtered to give 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (250 mg) as a yellow solid.

LC-MS (ESI): m/z=790.4 [M+H]⁺

### Step 6: tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate

To a solution of 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (250 mg, 316.46 mol, 1.0 equiv.) and 3-(6-amino-1-methyl-indazol-3-yl)piperidine-2,6-dione (122.60 mg, 474.70 mol, 1.5 equiv.) in pyridine (3 mL) was added EDCI (121.33 mg, 632.93 mol, 2.0 equiv.). The mixture was stirred at 60° C. for 1 hour. DCM (200 mL) and water (200 mL) were added, and the layers were separated. The aqueous layer was extracted with DCM (100 mL), dried over anhydrous sodium sulfate, filtered, and concentrated under vacuum to give tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (250 mg) as a yellow solid.

LC-MS (ESI): m/z=1030.5 [M+H]⁺

### Step 7: 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (250 mg, 242.66 mol, 1.0 equiv.) in DCM (1 mL) was added TFA (2 mL). The mixture was stirred at 25° C. for 12 hours. The reaction mixture was concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid (147.09 mg) as a yellow solid.

LC-MS (ESI): m/z=974.3 [M+H]⁺

¹H NMR (400 MHz, DMSO-d₆) δ=13.08-12.29 (m, 2H), 10.88 (s, 1H), 8.03 (d, 2H), 7.79 (d, J=8.0 Hz, 1H), 7.66-7.61 (m, 2H), 7.49-7.43 (m, 3H), 7.40-7.32 (m, 2H), 7.18 (d, J=8.4 Hz, 1H), 6.98-6.87 (m, 2H), 6.79 (d, J=2.4 Hz, 1H), 6.73-6.67 (m, 1H), 4.97 (s, 2H), 4.37-4.29 (m, 1H), 4.03-3.85 (m, 8H), 3.02 (t, J=6.0 Hz, 2H), 2.73-2.56 (m, 3H), 2.42 (s, 2H), 2.41-2.28 (m, 2H), 2.22-2.14 (m, 1H), 2.02 (s, 3H), 1.79-1.66 (m, 4H), 1.51-1.20 (m, 8H)

### Example 26: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid (Compound 132)

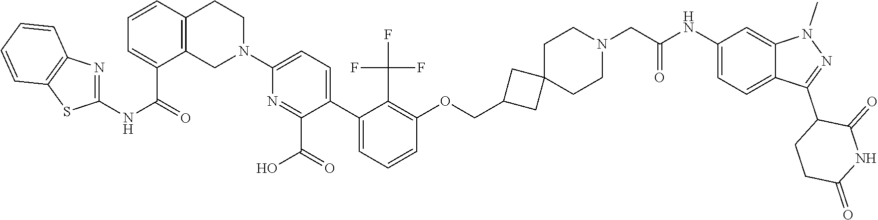 [SMILES: Cn1nc(C2CCC(=O)NC2=O)c2ccc(NC(=O)CN3CCC4(CC3)CC(COc3cccc(-c5ccc(N6CCc7cccc(C(=O)Nc8nc9ccccc9s8)c7C6)nc5C(=O)O)c3C(F)(F)F)C4)cc21]

### Step 1: tert-butyl 2-((3-bromo-2-(trifluoromethyl)phenoxy)methyl)-7-azaspiro[3.5]nonane-7-carboxylate

A mixture of tert-butyl 2-(hydroxymethyl)-7-azaspiro[3.5]nonane-7-carboxylate (600 mg, 2.35 mmol, 1 equiv.), 3-bromo-2-(trifluoromethyl)phenol (566.29 mg, 2.35 mmol, 1 equiv.), and 2-(tributyl-phosphanylidene)acetonitrile (737.24 mg, 3.05 mmol, 1.3 equiv.) in toluene (10 mL) was degassed and purged with N₂ three times. The mixture was stirred at 120° C. for 16 hours under a N₂ atmosphere. The combined organic layers were filtered and concentrated under reduced pressure to give a residue, which was purified by flash silica gel chromatography (0~20% ethyl acetate/petroleum ether) to afford tert-butyl 2-((3-bromo-2-(trifluoromethyl)phenoxy)methyl)-7-azaspiro[3.5]nonane-7-carboxylate (1 g) as a yellow oil.

LC-MS (ESI): m/z=422.1 [M−56+H]⁺.

¹H NMR (400 MHz, DMSO-d₆) δ=7.51-7.43 (m, 1H), 7.41-7.33 (m, 1H), 7.27 (d, J=8.4 Hz, 1H), 4.06 (d, J=5.2 Hz, 2H), 3.27 (s, 2H), 3.17 (s, 2H), 2.74-2.62 (m, 1H), 1.86 (t, J=10.4 Hz, 2H), 1.70-1.62 (m, 2H), 1.52 (s, 2H), 1.38 (s, 11H)

### Step 2: 2-[[3-bromo-2-(trifluoromethyl)phenoxy]methyl]-7-azaspiro[3.5]nonane

A mixture of tert-butyl 2-((3-bromo-2-(trifluoromethyl)phenoxy)methyl)-7-azaspiro[3.5]nonane-7-carboxylate (1 g, 2.09 mmol, 1 equiv.) in HCl/dioxane (10 mL) was stirred at 25° C. for 1 hour. The combined organic layers were filtered and concentrated under reduced pressure to afford 2-[[3-bromo-2-(trifluoromethyl)phenoxy]methyl]-7-azaspiro[3.5]nonane (950 mg, crude) as a white solid.

LC-MS (ESI): m/z=378.1 [M+H]⁺

### Step 3: ethyl 2-[2-[[3-bromo-2-(trifluoromethyl)phenoxy]methyl]-7-azaspiro[3.5]nonan-7-yl]acetate

A mixture of 2-[[3-bromo-2-(trifluoromethyl)phenoxy]methyl]-7-azaspiro[3.5]nonane (950 mg, 2.29 mmol, 1 equiv., HCl), ethyl 2-bromoacetate (382.58 mg, 2.29 mmol, 253.53 μL, 1 equiv.), and TEA (1.16 g, 11.45 mmol, 1.59 mL, 5 equiv.) in acetone (10 mL) was stirred at 25° C. for 1 hour. The combined organic layers were filtered and concentrated under reduced pressure to give a residue, which was purified by flash silica gel chromatography (0~20% ethyl acetate/petroleum ether) to afford ethyl 2-[2-[[3-bromo-2-(trifluoromethyl)phenoxy]methyl]-7-azaspiro[3.5]nonan-7-yl]acetate (600 mg) as a yellow oil.

LC-MS (ESI): m/z=464.1 [M+H]⁺

¹H NMR (400 MHz, DMSO-d₆) δ=7.49-7.43 (m, 1H), 7.37 (d, J=7.6 Hz, 1H), 7.26 (d, J=8.4 Hz, 1H), 4.09-4.02 (m, 4H), 3.12 (s, 2H), 2.70-2.62 (m, 1H), 2.42 (s, 2H), 2.32 (s, 2H), 1.82 (t, J=10.4 Hz, 2H), 1.65-1.56 (m, 4H), 1.46 (s, 2H), 1.17 (t, J=7.2 Hz, 3H)

### Step 4: tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-(trifluoromethyl)phenyl)picolinate

A mixture of ethyl 2-[2-[[3-bromo-2-(trifluoromethyl)phenoxy]methyl]-7-azaspiro[3.5]nonan-7-yl]acetate (250 mg, 538.43 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (395.77 mg, 646.11 mol, 1.2 equiv.), Ad₂nBup Pd G₃ (78.42 mg, 107.69 mol, 0.2 equiv.), and potassium carbonate (1.5 M, 1.08 mL, 3 equiv.) in dioxane (8 mL) was degassed and purged with N₂ three times. The mixture was stirred at 100° C. for 1 hour under a N₂ atmosphere in a microwave reactor. The reaction mixture was filtered and concentrated under reduced pressure to give a residue, which was purified by flash silica gel chromatography (0~40% ethyl acetate/petroleum ether) to afford tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-(trifluoromethyl)phenyl)picolinate (300 mg) as a yellow solid.

LC-MS (ESI): m/z=870.5 [M+H]⁺

¹H NMR (400 MHz, DMSO-d₆) δ=12.86 (s, 1H), 8.02 (d, J=8.0 Hz, 1H), 7.78 (d, J=8.0 Hz, 1H), 7.60 (d, J=7.6 Hz, 1H), 7.51-7.33 (m, 6H), 7.22 (d, J=8.4 Hz, 1H), 6.95 (d, J=8.8 Hz, 1H), 6.65 (d, J=7.6 Hz, 1H), 5.06-4.93 (m, 2H), 4.08-4.02 (m, 4H), 3.91-3.84 (m, 2H), 3.13 (s, 2H), 3.03 (t, J=5.6 Hz, 2H), 2.42 (s, 2H), 2.32 (d, J=1.6 Hz, 2H), 1.84-1.78 (m, 2H), 1.69-1.61 (m, 3H), 1.58 (s, 2H), 1.46 (s, 2H), 1.17 (t, J=7.2 Hz, 3H), 1.03 (s, 9H)

### Step 5: 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)methyl)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-(trifluoromethyl)phenyl)picolinate (300 mg, 344.83 mol, 1 equiv.) and LiOH H₂O (43.41 mg, 1.03 mmol, 3 equiv.) in THE (3 mL) and H₂O (1 mL) was stirred at 40° C. for 2 hours. The reaction mixture was diluted with H₂O (5 mL) and concentrated as a turbid liquid. The pH of the turbid liquid was adjusted to pH=3 with 1 N HCl (5 mL). The residue was extracted with DCM/MeOH (10:1). The combined organic layers were filtered and concentrated to afford 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)methyl)-7-azaspiro[3.5]nonan-7-yl)acetic acid (270 mg) as a yellow solid.

LC-MS (ESI): m/z=842.3 [M+H]⁺

### Step 6: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate

A mixture of 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)methyl)-7-azaspiro[3.5]nonan-7-yl)acetic acid (220 mg, 261.30 mol, 1 equiv.), 3-(6-amino-1-methyl-indazol-3-yl)piperidine-2,6-dione (80.99 mg, 313.56 mol, 1.2 equiv.), and EDCI (75.14 mg, 391.95 mol, 1.5 equiv.) in pyridine (3 mL) was stirred at 40° C. for 1 hour. The reaction mixture was diluted with H₂O (5 mL) and concentrated as a turbid liquid. The residue was extracted with DCM/MeOH (10:1), and the combined organic layers were filtered and concentrated to afford tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate (300 mg, crude) as a red solid.

LC-MS (ESI): m/z=1082.2 [M+H]⁺

### Step 7: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid

A mixture of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinate (300 mg, 277.21 mol, 1 equiv.) in DCM (1.5 mL) and TFA (1.5 mL) was stirred at 40° C. for 1 hour. The reaction mixture was filtered and concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-(trifluoromethyl)phenyl)picolinic acid (97.39 mg) as a yellow solid.

LC-MS (ESI): m/z=1026.3 [M+H]⁺

¹H NMR (400 MHz, DMSO-d₆) δ=13.17-12.72 (m, 1H), 12.67-12.10 (m, 1H), 10.88 (s, 1H), 10.65-9.63 (m, 1H), 8.13 (s, 1H), 8.07-7.98 (m, 2H), 7.79 (d, J=8.0 Hz, 1H), 7.64 (dd, J=8.8, 11.2 Hz, 2H), 7.50-7.34 (m, 6H), 7.19 (d, J=8.0 Hz, 2H), 6.99 (d, J=9.2 Hz, 1H), 6.68 (d, J=7.2 Hz, 1H), 5.05-4.93 (m, 2H), 4.33 (dd, J=4.8, 10.0 Hz, 1H), 4.06 (s, 2H), 3.93 (s, 5H), 3.50-3.40 (m, 2H), 3.03 (s, 2H), 2.74-2.60 (m, 4H), 2.42-2.26 (m, 2H), 2.17 (dd, J=5.6, 12.4 Hz, 1H), 2.06-1.85 (m, 3H), 1.83-1.58 (m, 7H)

### Example 27: 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid (Compound 134)

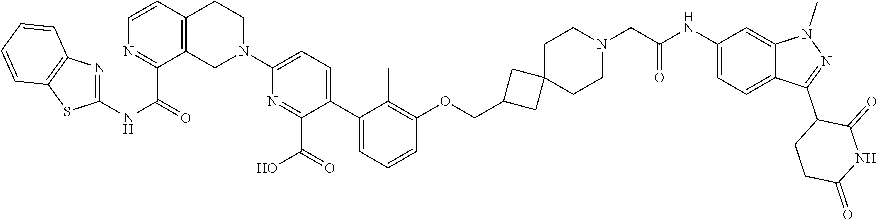 [SMILES: Cc1c(OCC2CC3(CCN(CC(=O)Nc4ccc5c(C6CCC(=O)NC6=O)nn(C)c5c4)CC3)C2)cccc1-c1ccc(N2CCc3ccnc(C(=O)Nc4nc5ccccc5s4)c3C2)nc1C(=O)O]

### Step 1: 2-(2-((3-bromo-2-methylphenoxy)methyl)-7-azaspiro[3.5]nonan-7-yl)acetic acid

A mixture of ethyl 2-(2-((3-bromo-2-methylphenoxy)methyl)-7-azaspiro[3.5]nonan-7-yl)acetate (1 g, 2.44 mmol, 1 equiv.) and LiOH H₂O (511.32 mg, 12.18 mmol, 5 equiv.) in H₂O (2 mL) and THE (8 mL) was stirred at 40° C. for 16 hours under a N₂ atmosphere. The reaction mixture was concentrated under reduced pressure to remove solvent. The pH of the residue was adjusted to pH=5-6 by HCl (1 M) and extracted with DCM (15 mL). The combined organic layers were dried over sodium sulfate, filtered, and concentrated under reduced pressure to afford 2-(2-((3-bromo-2-methylphenoxy)methyl)-7-azaspiro[3.5]nonan-7-yl)acetic acid (940 mg) as a yellow solid.

LC-MS (ESI): m/z=381.9 [M+H]⁺

¹H NMR (400 MHz, DMSO-d₆) δ=7.18-7.13 (m, 1H), 7.09 (t, J=8.0 Hz, 1H), 6.96 (d, J=8.0 Hz, 1H), 3.95 (d, J=6.0 Hz, 2H), 3.18 (s, 2H), 2.88 (s, 2H), 2.79 (s, 2H), 2.71-2.64 (m, 1H), 2.23 (s, 3H), 1.96-1.88 (m, 2H), 1.76 (t, J=5.2 Hz, 2H), 1.70-1.63 (m, 4H).

### Step 2: 2-(2-((3-bromo-2-methylphenoxy)methyl)-7-azaspiro[3.5]nonan-7-yl)-N-(3-(2,6-dioxopiperidin-3-yl)-1-methyl-1H-indazol-6-yl)acetamide

A mixture of 2-(2-((3-bromo-2-methylphenoxy)methyl)-7-azaspiro[3.5]nonan-7-yl)acetic acid (920 mg, 2.41 mmol, 1 equiv.), 3-(6-amino-1-methyl-indazol-3-yl)piperidine-2,6-dione (745.86 mg, 2.89 mmol, 1.2 equiv.), and EDCI (692.01 mg, 3.61 mmol, 1.5 equiv.) in pyridine (10 mL) was degassed and purged with N₂ three times. The mixture was stirred at 25° C. for 16 hours. The reaction mixture was diluted with H₂O (10 mL) and extracted with ethyl acetate (45 mL). The combined organic layers were washed with brine (45 mL), dried over sodium sulfate, filtered, and concentrated under reduced pressure to give a residue, which was purified by flash silica gel chromatography (0-88% ethyl acetate/petroleum ether) to afford 2-(2-((3-bromo-2-methylphenoxy)methyl)-7-azaspiro[3.5]nonan-7-yl)-N-(3-(2,6-dioxopiperidin-3-yl)-1-methyl-1H-indazol-6-yl)acetamide (1.06 g) as a yellow solid.

LC-MS (ESI): m/z=622.2 [M+H]⁺

¹H NMR (400 MHz, DMSO-d₆) δ=10.88 (s, 1H), 9.82 (s, 1H), 8.05 (s, 1H), 7.63 (d, J=8.8 Hz, 1H), 7.22 (d, J=8.8, 1.2 Hz, 1H), 7.18-7.13 (m, 1H), 7.12-7.06 (m, 1H), 6.97 (d, J=8.0 Hz, 1H), 4.32 (d, J=9.6, 5.2 Hz, 1H), 3.96 (d, J=6.0 Hz, 2H), 3.92 (s, 3H), 3.11 (s, 2H), 2.73-2.60 (m, 3H), 2.48-2.45 (m, 1H), 2.44-2.28 (m, 4H), 2.24 (s, 3H), 2.17 (d, J=13.6, 5.2 Hz, 1H), 1.96-1.88 (m, 2H), 1.72-1.68 (m, 2H), 1.65 (d, J=11.6 Hz, 2H), 1.60 (t, J=5.2 Hz, 2H).

### Step 3: N-(3-(2,6-dioxopiperidin-3-yl)-1-methyl-1H-indazol-6-yl)-2-(2-((2-methyl-3-(4,4,5,5-tetramethyl-1,3,2-dioxaborolan-2-yl)phenoxy)methyl)-7-azaspiro[3.5]nonan-7-yl)acetamide

A mixture of 2-(2-((3-bromo-2-methylphenoxy)methyl)-7-azaspiro[3.5]nonan-7-yl)-N-(3-(2,6-dioxopiperidin-3-yl)-1-methyl-1H-indazol-6-yl)acetamide (1 g, 1.61 mmol, 1 equiv.), 4,4,5,5-tetramethyl-2-(4,4,5,5-tetramethyl-1,3,2-dioxaborolan-2-yl)-1,3,2-dioxaborolane (693.43 mg, 2.73 mmol, 1.7 equiv.), KOAc (472.94 mg, 4.82 mmol, 3 equiv.), and Pd(dppf)Cl₂ (117.53 mg, 160.63 mol, 0.1 equiv.) in dioxane (10 mL) was degassed and purged with N₂ three times. The mixture was stirred at 90° C. for 16 hours under a N₂ atmosphere. The reaction mixture was concentrated under reduced pressure to give a residue, which was purified by flash silica gel chromatography (0-80% ethyl acetate/petroleum ether) to afford N-(3-(2,6-dioxopiperidin-3-yl)-1-methyl-1H-indazol-6-yl)-2-(2-((2-methyl-3-(4,4,5,5-tetramethyl-1,3,2-dioxaborolan-2-yl)phenoxy)methyl)-7-azaspiro[3.5]nonan-7-yl)acetamide (1 g) as a yellow solid.

LC-MS (ESI): m/z=670.4 [M+H]⁺

¹H NMR (400 MHz, DMSO-d₆) δ=10.87 (s, 1H), 9.82 (s, 1H), 8.04 (s, 1H), 7.63 (d, J=8.4 Hz, 1H), 7.22 (d, J=8.8, 1.2 Hz, 1H), 7.19-7.15 (m, 1H), 7.11 (t, J=7.6 Hz, 1H), 7.01 (d, J=7.6 Hz, 1H), 4.32 (d, J=9.6, 5.2 Hz, 1H), 3.94-3.88 (m, 5H), 3.10 (s, 2H), 2.70-2.60 (m, 3H), 2.47-2.46 (m, 1H), 2.45-2.34 (m, 4H), 2.33 (s, 3H), 2.20-2.14 (m, 1H), 1.94-1.87 (m, 2H), 1.72-1.63 (m, 4H), 1.63-1.57 (m, 2H), 1.29 (s, 12H).

### Step 4: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate

A mixture of N-(3-(2,6-dioxopiperidin-3-yl)-1-methyl-1H-indazol-6-yl)-2-(2-((2-methyl-3-(4,4,5,5-tetramethyl-1,3,2-dioxaborolan-2-yl)phenoxy)methyl)-7-azaspiro[3.5]nonan-7-yl)acetamide (50 mg, 74.67 mol, 1 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (62.43 mg, 89.60 mol, 1.2 equiv.), KF (3 M, 74.67 μL, 3 equiv.), and Ad₂nBup Pd G₃ (5.44 mg, 7.47 mol, 0.1 equiv.) in dioxane (1 mL) was degassed and purged with N₂ three times. The mixture was stirred at 100° C. for 4 hours under a N₂ atmosphere. The reaction mixture was concentrated to give a residue, which was purified by flash column chromatography (petroleum ether/ethyl acetate=1/0 to DCM/MeOH=10/1) and revised-phase chromatography (FA condition) to afford tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (100 mg) as a yellow solid.

LC-MS (ESI): m/z=1159.5 [M+H]⁺

### Step 5: 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid

A solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (100 mg, 86.25 mol, 1 equiv.) in TFA (1 mL) and DCM (1 mL). The mixture was stirred at 25° C. for 12 hours was concentrated under reduced pressure to give a residue, which was purified by prep-HPLC to afford 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid (29.96 mg) as a yellow solid.

LC-MS (ESI): m/z=973.3 [M+H]⁺

¹H NMR (400 MHz, DMSO-d₆) δ=12.79-12.19 (m, 2H), 10.88 (s, 1H), 9.89 (s, 1H), 8.64-8.48 (m, 1H), 8.13 (s, 1H), 8.08-8.02 (m, 2H), 7.82 (d, J=8.4 Hz, 1H), 7.63 (d, J=8.8 Hz, 1H), 7.59 (d, J=2.4 Hz, 1H), 7.52-7.45 (m, 2H), 7.39-7.33 (m, 1H), 7.21 (d, J=9.2 Hz, 1H), 7.15-7.03 (m, 2H), 6.90 (d, J=8.4 Hz, 1H), 6.65 (d, J=8.0 Hz, 1H), 5.25 (s, 2H), 4.36-4.29 (m, 1H), 3.99-3.91 (m, 7H), 3.08 (d, J=5.2 Hz, 2H), 2.73-2.59 (m, 5H), 2.39-2.31 (m, 2H), 2.24-2.11 (m, 2H), 1.97-1.91 (m, 5H), 1.74-1.67 (m, 4H), 1.65-1.60 (m, 2H), 1.33-1.16 (m, 2H).

### Example 28: 6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid] (Compound 133a)

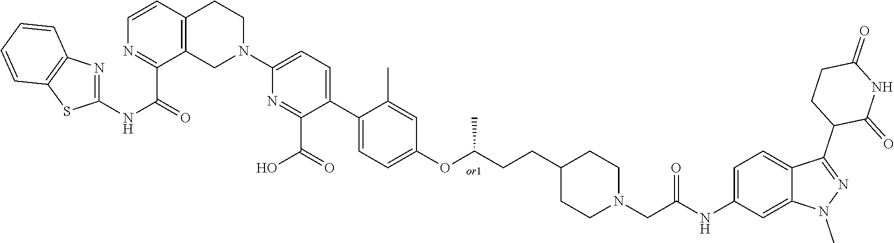 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O]

### Step 1: (R)-4-(3-(4-bromo-3-methylphenoxy)butyl)piperidine

To a solution of tert-butyl (R)-4-(3-(4-bromo-3-methylphenoxy)butyl)piperidine-1-carboxylate (2.3 g, 5.39 mmol, 1.0 equiv.) in EtOAc (12 mL) was added HCl/dioxane (4 M, 12 mL, 8.9 equiv.). The mixture was stirred at 25° C. for 1 hour. The mixture was concentrated under reduced pressure to give (R)-4-(3-(4-bromo-3-methylphenoxy)butyl)piperidine (2 g, crude, HCl) as an off-white solid.

LC-MS (ESI): m/z=326.0 [M+H]⁺

### Step 2: ethyl (R)-2-(4-(3-(4-bromo-3-methylphenoxy)butyl)piperidin-1-yl)acetate

To a solution of (R)-4-(3-(4-bromo-3-methylphenoxy)butyl)piperidine (2 g, 5.51 mmol, 1.0 equiv., HCl) in DMF (20 mL) was added potassium carbonate (2.29 g, 16.54 mmol, 3.0 equiv.) and ethyl 2-bromoacetate (828.72 mg, 4.96 mmol, 549.18 μL, 0.9 equiv.). The mixture was stirred at 40° C. for 1 hour. The reaction mixture was diluted with H₂O (50 mL) and extracted with EtOAc (300 mL). The combined organic layers were washed with H₂O (300 mL), dried over sodium sulfate, filtered, and concentrated under reduced pressure to give a residue, which was purified by flash silica gel chromatography (0-25% ethyl acetate/petroleum ether) to give ethyl (R)-2-(4-(3-(4-bromo-3-methylphenoxy)butyl)piperidin-1-yl)acetate (2 g) as a yellow oil.

LC-MS (ESI): m/z=412.0 [M+H]⁺

¹H NMR (400 MHz, DMSO-d₆) δ=7.41 (d, J=8.4 Hz, 1H), 6.92 (s, 1H), 6.69 (d, J=8.4 Hz, 1H), 4.43-4.33 (m, 1H), 4.11-4.01 (m, 2H), 3.14 (s, 2H), 2.77 (d, J=10.4 Hz, 2H), 2.28 (s, 3H), 2.09 (t, J=10.4 Hz, 2H), 1.69-1.46 (m, 4H), 1.37-1.28 (m, 1H), 1.27-1.01 (m, 10H)

### Step 3: (R)-2-(4-(3-(4-bromo-3-methylphenoxy)butyl)piperidin-1-yl)acetic acid

To a solution of ethyl (R)-2-(4-(3-(4-bromo-3-methylphenoxy)butyl)piperidin-1-yl)acetate (1 g, 2.43 mmol, 1.0 equiv.) in THE (15 mL) and H₂O (5 mL) was added LiOH·H₂O (305.29 mg, 7.28 mmol, 3.0 equiv.). The mixture was stirred at 25° C. for 2 hours. The reaction mixture was concentrated under reduced pressure to remove THF (15 mL). The residue was diluted with H₂O (20 mL), adjusted to pH=3 with HCl (1 M), and extracted with EtOAc (90 mL). The combined organic layers were washed with brine (30 mL), dried over sodium sulfate, filtered, and concentrated under reduced pressure to give (R)-2-(4-(3-(4-bromo-3-methylphenoxy)butyl)piperidin-1-yl)acetic acid (0.9 g, crude) as a light brown oil.

LC-MS (ESI): m/z=385.9 [M+2+H]⁺ (⁸⁰Br).

¹H NMR (400 MHz, DMSO-d₆) δ=7.42 (d, J=8.8 Hz, 1H), 6.92 (s, 1H), 6.71 (d, J=8.8 Hz, 1H), 4.47-4.36 (m, 1H), 3.83 (s, 2H), 3.41-3.37 (m, 2H), 2.94-2.84 (m, 2H), 2.29 (s, 3H), 1.84-1.73 (m, 2H), 1.66-1.51 (m, 2H), 1.47-1.24 (m, 5H), 1.20 (d, J=6.0 Hz, 3H)

### Step 4: 2-(4-((R)-3-(4-bromo-3-methylphenoxy)butyl)piperidin-1-yl)-N-(3-(2,6-dioxopiperidin-3-yl)-1-methyl-1H-indazol-6-yl)acetamide

To a solution of (R)-2-(4-(3-(4-bromo-3-methylphenoxy)butyl)piperidin-1-yl)acetic acid (0.8 g, 2.08 mmol, 1.0 equiv.) in pyridine (20 mL) was added 3-(6-amino-1-methyl-indazol-3-yl)piperidine-2,6-dione (674.90 mg, 2.29 mmol, 1.1 equiv., HCl) and EDCI (478.87 mg, 2.50 mmol, 1.2 equiv.). The mixture was stirred at 40° C. for 1 hour. The reaction mixture was diluted with H₂O (50 mL) and extracted with EtOAc (300 mL). The combined organic layers were washed with brine (200 mL), dried over sodium sulfate, filtered, and concentrated under reduced pressure to give a residue, which was purified by flash silica gel chromatography (0~90% ethyl acetate/petroleum ether) to give 2-(4-((R)-3-(4-bromo-3-methylphenoxy)butyl)piperidin-1-yl)-N-(3-(2,6-dioxopiperidin-3-yl)-1-methyl-1H-indazol-6-yl)acetamide (890 mg) as a yellow solid.

LC-MS (ESI): m/z=624.2 [M+2+H]⁺

¹H NMR (400 MHz, DMSO-d₆) δ=10.88 (s, 1H), 9.81 (s, 1H), 8.06-8.03 (m, 1H), 7.63 (d, J=8.8 Hz, 1H), 7.41 (d, J=8.8 Hz, 1H), 7.21 (dd, J=1.6, 8.8 Hz, 1H), 6.93 (d, J=2.8 Hz, 1H), 6.74-6.70 (m, 1H), 4.44-4.37 (m, 1H), 4.35-4.29 (m, 1H), 3.92 (s, 3H), 3.12 (s, 2H), 2.86 (d, J=10.8 Hz, 2H), 2.68-2.60 (m, 2H), 2.36-2.31 (m, 1H), 2.29 (s, 3H), 2.20-2.09 (m, 3H), 1.69-1.60 (m, 3H), 1.60-1.53 (m, 1H), 1.41-1.33 (m, 1H), 1.31-1.20 (m, 7H)

### Step 5: N-(3-(2,6-dioxopiperidin-3-yl)-1-methyl-1H-indazol-6-yl)-2-(4-((R)-3-(3-methyl-4-(4,4,5,5-tetramethyl-1,3,2-dioxaborolan-2-yl)phenoxy)butyl)piperidin-1-yl)acetamide

A mixture of 2-(4-((R)-3-(4-bromo-3-methylphenoxy)butyl)piperidin-1-yl)-N-(3-(2,6-dioxopiperidin-3-yl)-1-methyl-1H-indazol-6-yl)acetamide (870 mg, 1.39 mmol, 1.0 equiv.), bis(pinacolato)diboron (530.59 mg, 2.09 mmol, 1.5 equiv.), and KOAc (410.13 mg, 4.18 mmol, 3.0 equiv.) in dioxane (13 mL) was degassed and purged with N₂ three times. To this mixture was added Pd(dppf)Cl₂·CH₂Cl₂ (113.75 mg, 139.30 mol, 0.1 equiv.). The mixture was stirred at 90° C. for 16 hours under a N₂ atmosphere. The mixture was concentrated under reduced pressure to give a residue, which was purified by flash silica gel chromatography (0-80% ethyl acetate/petroleum ether) and reversed-phase HPLC to give N-(3-(2,6-dioxopiperidin-3-yl)-1-methyl-1H-indazol-6-yl)-2-(4-((R)-3-(3-methyl-4-(4,4,5,5-tetramethyl-1,3,2-dioxaborolan-2-yl)phenoxy)butyl)piperidin-1-yl)acetamide (450 mg) as a yellow solid.

LC-MS (ESI): m/z=672.4 [M+H]⁺

### Step 6: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate

A mixture of N-(3-(2,6-dioxopiperidin-3-yl)-1-methyl-1H-indazol-6-yl)-2-(4-((R)-3-(3-methyl-4-(4,4,5,5-tetramethyl-1,3,2-dioxaborolan-2-yl)phenoxy)butyl)piperidin-1-yl)acetamide (190 mg, 282.89 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (236.52 mg, 339.47 mol, 1.2 equiv.), and KF (1.5 M, 1.13 mL, 6.0 equiv.) in dioxane (8 mL) was degassed and purged with N₂ three times. To this mixture was added PdCl₂(dcypf) (18.44 mg, 28.29 mol, 0.1 equiv.). The mixture was stirred at 90° C. for 16 hours under a N₂ atmosphere. The mixture was concentrated under reduced pressure to give a residue, which was purified by flash silica gel chromatography (0~80% ethyl acetate/petroleum ether) and reversed-phase HPLC to give tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate (43 mg) as a yellow oil.

LC-MS (ESI): m/z=1061.7 [M+H]⁺

### Step 7: 6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

To a solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinate (43 mg, 37.02 mol, 1.0 equiv.) in DCM (1 mL) was added TFA (767.50 mg, 6.73 mmol, 0.5 mL, 181.82 equiv.). The mixture was stirred at 25° C. for 16 hours. The mixture was concentrated under reduced pressure to give a residue, which was purified by prep-HPLC to give 6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid [rel-6-(8-(benzo[d]thiazol-2-ylcarbamoyl)-3,4-dihydro-2,7-naphthyridin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid] (9.22 mg) as a white solid.

LC-MS (ESI): m/z=975.4 [M+H]⁺

¹H NMR (400 MHz, DMSO-d₆) δ=12.43-12.16 (m, 1H), 10.88 (s, 1H), 10.01-9.76 (m, 1H), 8.57 (d, J=4.8 Hz, 1H), 8.08-8.02 (m, 2H), 7.82 (d, J=8.0 Hz, 1H), 7.63 (d, J=8.4 Hz, 1H), 7.58 (d, J=5.2 Hz, 1H), 7.53-7.45 (m, 2H), 7.39-7.34 (m, 1H), 7.21 (dd, J=1.2, 8.8 Hz, 1H), 7.05 (d, J=8.8 Hz, 1H), 6.93 (d, J=8.4 Hz, 1H), 6.80-6.76 (m, 1H), 6.74-6.68 (m, 1H), 5.24 (s, 2H), 4.44-4.38 (m, 1H), 4.32 (dd, J=4.8, 9.6 Hz, 1H), 3.97 (t, J=5.6 Hz, 2H), 3.92 (s, 3H), 3.22-3.13 (m, 2H), 3.08 (t, J=5.6 Hz, 2H), 2.97-2.84 (m, 2H), 2.69-2.60 (m, 2H), 2.40-2.26 (m, 2H), 2.21-2.13 (m, 2H), 2.04 (s, 3H), 1.73-1.63 (m, 3H), 1.61-1.53 (m, 1H), 1.44-1.36 (m, 1H), 1.35-1.25 (m, 4H), 1.24 (d, J=6.0 Hz, 3H)

### Example 29: 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid] (Compound 138b)

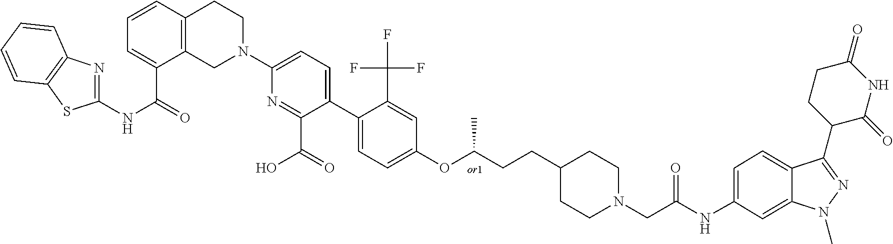 [SMILES: C[C@H](CCC1CCN(CC(=O)Nc2ccc3c(C4CCC(=O)NC4=O)nn(C)c3c2)CC1)Oc1ccc(-c2ccc(N3CCc4cccc(C(=O)Nc5nc6ccccc6s5)c4C3)nc2C(=O)O)c(C(F)(F)F)c1]

### Step 1: (R)-4-(3-(4-bromo-3-(trifluoromethyl)phenoxy)butyl)piperidine

A solution of tert-butyl (R)-4-(3-(4-bromo-3-(trifluoromethyl)phenoxy)butyl)piperidine-1-carboxylate (630 mg, 1.31 mmol, 1 equiv.) in HCl/dioxane (6.3 mL, 4 M) was stirred at 25° C. for 12 hours. The reaction mixture was concentrated under reduced pressure to give (R)-4-(3-(4-bromo-3-(trifluoromethyl)phenoxy)butyl)piperidine (520 mg, crude) as a colorless oil.

LC-MS (ESI): m/z=380.0 [M+H]⁺

### Step 2: ethyl (R)-2-(4-(3-(4-bromo-3-(trifluoromethyl)phenoxy)butyl)piperidin-1-yl)acetate

To a solution of (R)-4-(3-(4-bromo-3-(trifluoromethyl)phenoxy)butyl)piperidine (500 mg, 1.31 mmol, 1 equiv.) in acetone (5 mL) was added TEA (399.18 mg, 3.94 mmol, 549.07 L, 3 equiv.) and ethyl 2-bromoacetate (219.60 mg, 1.31 mmol, 145.53 μL, 1 equiv.). The mixture was stirred at 0° C. for 0.1 hours. The reaction mixture was concentrated under reduced pressure to give a residue, which was purified by flash silica gel chromatography (0-100% ethyl acetate/petroleum ether) to give ethyl (R)-2-(4-(3-(4-bromo-3-(trifluoromethyl)phenoxy)butyl)piperidin-1-yl)acetate (500 mg) as a colorless.

LC-MS (ESI): m/z=468.0 [M+H]⁺

### Step 3: tert-butyl (R)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate

To a solution of ethyl (R)-2-(4-(3-(4-bromo-3-(trifluoromethyl)phenoxy)butyl)piperidin-1-yl)acetate (200 mg, 428.88 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (288.98 mg, 471.77 mol, 1.1 equiv.) in dioxane (2 mL) and H₂O (0.2 mL) was added Ad₂nBup Pd G₃ (31.23 mg, 42.89 mol, 0.1 equiv.) and KF (74.75 mg, 1.29 mmol, 30.14 μL, 3 equiv.). The mixture was stirred at 100° C. for 3 hours. The reaction mixture was concentrated under reduced pressure to give a residue, which was purified by flash silica gel chromatography (0-10% dichloromethane/methanol) to give tert-butyl (R)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate (400 mg) as a yellow solid.

LC-MS (ESI): m/z=872.3 [M+H]⁺

### Step 4: (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid

To a solution of tert-butyl (R)-6-(8-(benzo[d]thiazol-2-ylcarbamoyl)-3,4-dihydroisoquinolin-2(1H)-yl)-3-(4-((4-(1-(2-ethoxy-2-oxoethyl)piperidin-4-yl)butan-2-yl)oxy)-2-(trifluoromethyl)phenyl)picolinate (400.00 mg, 458.71 mol, 1 equiv.) in THF (4 mL) and H₂O (0.4 mL) was added LiOH·H₂O (57.75 mg, 1.38 mmol, 3 equiv.). The mixture was stirred at 25° C. for 12 hours. The reaction mixture was adjusted to pH=4-5 with 1M HCl and filtered to give (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid (380 mg) as a white solid.

LC-MS (ESI): m/z=844.3 [M+H]⁺

### Step 5: tert-butyl 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate

To a solution of (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)butyl)piperidin-1-yl)acetic acid (380 mg, 450.26 mol, 1 equiv.) and 3-(6-amino-1-methyl-indazol-3-yl)piperidine-2,6-dione (159.25 mg, 540.32 mol, 1.2 equiv., HCl) in pyridine (4 mL) was added EDCI (129.47 mg, 675.39 mol, 1.5 equiv.). The mixture was stirred at 25° C. for 2 hours. The reaction mixture was partitioned between DCM (15 mL) and H₂O (5 mL). The organic phase was dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate (300 mg) as a white solid.

LC-MS (ESI): m/z=1084.3 [M+H]⁺

### Step 6: 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid]

To a solution of tert-butyl 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinate (300 mg, 276.70 mol, 1 equiv.) in DCM (2 mL) was added TFA (2 mL). The mixture was stirred at 25° C. for 12 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-(trifluoromethyl)phenyl)picolinic acid] (80.13 mg) as an off-white solid.

LC-MS (ESI): m/z=1028.4 [M+H]⁺

¹H NMR (400 MHz, DMSO-d₆) δ=13.04-12.70 (m, 1H), 10.88 (s, 1H), 10.19-9.52 (m, 1H), 8.11-7.92 (m, 2H), 7.87-7.72 (m, 1H), 7.67-7.58 (m, 2H), 7.50-7.42 (m, 3H), 7.41-7.31 (m, 2H), 7.23-7.13 (m, 4H), 6.99 (d, J=8.8 Hz, 1H), 5.11-4.88 (m, 2H), 4.62-4.49 (m, 1H), 4.32 (dd, J=5.2, 9.6 Hz, 1H), 3.98-3.88 (m, 5H), 3.05-2.84 (m, 4H), 2.75-2.51 (m, 4H), 2.39-2.13 (m, 4H), 1.75-1.57 (m, 4H), 1.42-1.23 (m, 8H).

### Example 30: 6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid](Compound 140b)

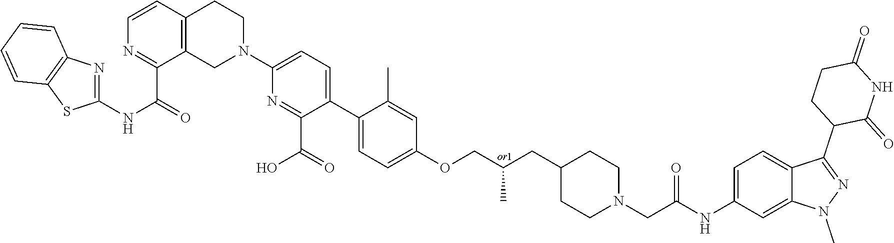 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3ccnc(C(=O)Nc4nc5ccccc5s4)c3C2)nc1C(=O)O]

### Step 1: tert-butyl 4-(3-ethoxy-2-methyl-3-oxo-propyl)piperidine-1-carboxylate

To a solution of tert-butyl 4-[(E)-3-ethoxy-2-methyl-3-oxo-prop-1-enyl]piperidine-1-carboxylate (3.0 g, 10.09 mmol, 1.0 equiv.) in MeOH (30 mL) was added Pd on carbon (10 wt %, 2.15 g, 2.02 mmol, 0.2 equiv.) and Pd(OH)₂ on carbon (20 wt %, 1.42 g, 2.02 mmol, 0.2 equiv.) under a N₂ atmosphere. The suspension was degassed and purged with H₂ three times. The mixture was stirred under H₂ (15 psi) at 40° C. for 16 hours. The reaction mixture was filtered and concentrated under reduced pressure to give tert-butyl 4-(3-ethoxy-2-methyl-3-oxo-propyl)piperidine-1-carboxylate (2.8 g) as a colorless oil.

LC-MS (ESI): m/z=200.0 [M+H−100]⁺.

### Step 2: tert-butyl 4-(3-hydroxy-2-methyl-propyl)piperidine-1-carboxylate

To a solution of tert-butyl 4-(3-ethoxy-2-methyl-3-oxo-propyl)piperidine-1-carboxylate (2.8 g, 9.35 mmol, 1.0 equiv.) in THF (30 mL) was added LAH (2.5 M, 4.49 mL, 1.2 equiv.) at 0° C. The mixture was stirred at 25° C. for 1 hour. The reaction mixture quenched with sodium sulfate·10 H₂O (10.0 g) at 0° C. and filtrate to give a filtrate. The filtrate was concentrated to give tert-butyl 4-(3-hydroxy-2-methyl-propyl)piperidine-1-carboxylate (2.2 g) as a colorless oil.

¹H NMR (400 MHz, DMSO-d₆) δ=4.37 (t, J=5.2 Hz, 1H), 3.90 (d, J=11.6 Hz, 2H), 3.25-3.21 (m, 1H), 3.17-3.12 (m, 1H), 2.67 (d, J=2.0 Hz, 2H), 1.62-1.56 (m, 3H), 1.47-1.45 (m, 1H), 1.38 (s, 9H), 1.26-1.19 (m, 1H), 0.95-0.87 (m, 3H), 0.81 (d, J=6.4 Hz, 3H)

### Step 3: tert-butyl 4-[3-(4-bromo-3-methyl-phenoxy)-2-methyl-propyl]piperidine-1-carboxylate

To a solution of tert-butyl 4-(3-hydroxy-2-methyl-propyl)piperidine-1-carboxylate (1.0 g, 3.89 mmol, 1.0 equiv.) in toluene (20 mL) was added 2-(tributyl-phosphanylidene)acetonitrile (1.41 g, 5.83 mmol, 1.5 equiv.), and 4-bromo-3-methyl-phenol (799.39 mg, 4.27 mmol, 1.1 equiv.). The mixture was stirred at 120° C. for 12 hours. The reaction mixture was concentrated under reduced pressure to give a residue, which was purified by column chromatography (SiO₂, petroleum ether/ethyl acetate=100/1 to 10/1) and reversed-phase HPLC to give tert-butyl 4-[3-(4-bromo-3-methyl-phenoxy)-2-methyl-propyl]piperidine-1-carboxylate (1.2 g) as a yellow oil.

LC-MS (ESI): m/z=326.0 [M+H]⁺

¹H NMR (400 MHz, CDCl₃) δ=7.39 (d, J=8.8 Hz, 1H), 6.78 (s, 1H), 6.60 (d, J=8.8 Hz, 1H), 4.09 (d, J=13.2 Hz, 2H), 3.76-3.67 (m, 2H), 2.74-2.65 (m, 2H), 2.36 (s, 3H), 2.06-2.03 (m, 1H), 1.67 (d, J=12.0 Hz, 2H), 1.57-1.52 (m, 2H), 1.46 (s, 9H), 1.43-1.39 (m, 1H), 1.18-1.05 (m, 3H), 1.02 (d, J=6.4 Hz, 3H)

### Step 4: tert-butyl 4-[(2S)-3-(4-bromo-3-methyl-phenoxy)-2-methyl-propyl]piperidine-1-carboxylate and tert-butyl 4-[(2R)-3-(4-bromo-3-methyl-phenoxy)-2-methyl-propyl]piperidine-1-carboxylate

The enantiomers of tert-butyl 4-[3-(4-bromo-3-methyl-phenoxy)-2-methyl-propyl]piperidine-1-carboxylate were separated by SFC (column: Daicel Chiralpak IC (250 mm×30 mm, 10 μm); mobile phase: [CO₂-iPrOH (0.1% NH₃H₂O)]; B %: 25%, isocratic elution mode) to give tert-butyl 4-[(2S)-3-(4-bromo-3-methyl-phenoxy)-2-methyl-propyl]piperidine-1-carboxylate (Peak 1) (490 mg) as a yellow oil and tert-butyl 4-[(2R)-3-(4-bromo-3-methyl-phenoxy)-2-methyl-propyl]piperidine-1-carboxylate (Peak 2) (450 mg) as a yellow oil.

LC-MS (ESI): m/z=326.0 [M+H]⁺

Peak 1:

SFC: t_{R}=1.338 mins

Peak 2:

SFC: t_{R}=1.680 mins

### Step 5: 4-[(2S)-3-(4-bromo-3-methyl-phenoxy)-2-methyl-propyl]piperidine

To a solution of tert-butyl 4-[(2S)-3-(4-bromo-3-methyl-phenoxy)-2-methyl-propyl]piperidine-1-carboxylate (950 mg, 2.23 mmol, 1.0 equiv.) in EtOAc (2 mL) was added HCl/dioxane (10 mL). The mixture was stirred at 25° C. for 1 hour. The reaction mixture was concentrated under reduced pressure to give 4-[(2S)-3-(4-bromo-3-methyl-phenoxy)-2-methyl-propyl]piperidine (750 mg) as a white solid, which was used for next step without purification.

LC-MS (ESI): m/z=326.1 [M+H]⁺

### Step 6: ethyl 2-[4-[(2S)-3-(4-bromo-3-methyl-phenoxy)-2-methyl-propyl]-1-piperidyl]acetate

To a solution of 4-[(2S)-3-(4-bromo-3-methyl-phenoxy)-2-methyl-propyl]piperidine (750 mg, 2.07 mmol, 1.0 equiv., HCl) in DMF (10 mL) and potassium carbonate (857.28 mg, 6.20 mmol, 3.0 equiv.) was added ethyl 2-bromoacetate (345.30 mg, 2.07 mmol, 228.83 μL, 1.0 equiv.). The mixture was stirred at 40° C. for 1 hour. Ethyl acetate (200 mL) and H₂O (100 mL) were added and layers were separated. The aqueous layer was extracted with ethyl acetate (100 mL), dried over anhydrous sodium sulfate, filtered, and concentrated under reduced pressure to give ethyl 2-[4-[(2S)-3-(4-bromo-3-methyl-phenoxy)-2-methyl-propyl]-1-piperidyl]acetate (840 mg) as a yellow oil, which was used in the next step without purification.

LC-MS (ESI): m/z=412.1 [M+H]⁺

### Step 7: 2-[4-[(2S)-3-(4-bromo-3-methyl-phenoxy)-2-methyl-propyl]-1-piperidyl]acetic acid

To a solution of ethyl 2-[4-[(2S)-3-(4-bromo-3-methyl-phenoxy)-2-methyl-propyl]-1-piperidyl]acetate (840 mg, 2.04 mmol, 1.0 equiv.) in THE (4 mL) was added LiOH·H₂O (256.42 mg, 6.11 mmol, 3.0 equiv.) and H₂O (0.8 mL). The mixture was stirred at 25° C. for 12 hours. The reaction mixture was diluted with water (5 mL) and the pH was adjusted to pH~6 by progressively adding 1M HCl. The mixture was filtered to give 2-[4-[(2S)-3-(4-bromo-3-methyl-phenoxy)-2-methyl-propyl]-1-piperidyl]acetic acid (780 mg, crude) as a white solid, which was used in the next step without purification.

LC-MS (ESI): m/z=384.1 [M+H]⁺

¹HNMR (400 MHz, DMSO-d₆) δ=7.43 (d, J=8.0 Hz, 1H), 6.95 (s, 1H), 6.71 (d, J=8.8 Hz, 1H), 3.81-3.77 (m, J=6.4 Hz, 2H), 3.73 (s, 2H), 3.38 (d, J=12.0 Hz, 2H), 2.89 (s, 2H), 2.30 (s, 3H), 2.02-1.88 (m, 1H), 1.79 (d, J=12.8 Hz, 2H), 1.59 (s, 1H), 1.44-1.35 (m, 3H), 1.17-1.09 (m, 1H), 0.96 (d, J=6.4 Hz, 3H)

### Step 8: 2-[4-[(2S)-3-(4-bromo-3-methyl-phenoxy)-2-methyl-propyl]-1-piperidyl]-N-[3-(2,6-dioxo-3-piperidyl)-1-methyl-indazol-6-yl]acetamide

A mixture of 2-[4-[(2S)-3-(4-bromo-3-methyl-phenoxy)-2-methyl-propyl]-1-piperidyl]acetic acid (360 mg, 936.75 mol, 1.0 equiv.), EDCI (269.36 mg, 1.41 mmol, 1.5 equiv.), and 3-(6-amino-1-methyl-indazol-3-yl)piperidine-2,6-dione (331.31 mg, 1.12 mmol, 1.2 equiv., HCl) in pyridine (5 mL) was degassed and purged with N₂ three times. The mixture was stirred at 60° C. for 1 hour under a N₂ atmosphere. The reaction mixture was concentrated under reduced pressure to give a residue, which was purified by column chromatography (SiO₂, petroleum ether/ethyl acetate=1/1 to DCM/MeOH=10/1) to give 2-[4-[(2S)-3-(4-bromo-3-methyl-phenoxy)-2-methyl-propyl]-1-piperidyl]-N-[3-(2,6-dioxo-3-piperidyl)-1-methyl-indazol-6-yl]acetamide (524 mg) as a yellow solid.

LC-MS (ESI): m/z=624.2 [M+H]⁺

¹HNMR (400 MHz, DMSO-d₆) δ=10.88 (s, 1H), 9.82 (s, 1H), 8.05 (s, 1H), 7.63 (d, J=8.8 Hz, 1H), 7.42 (d, J=8.8 Hz, 1H), 7.21 (d, J=8.8 Hz, 1H), 6.95 (s, 1H), 6.72 (d, J=8.8 Hz, 1H), 4.34-4.30 (m, 1H), 3.92 (s, 3H), 3.84-3.76 (m, 1H), 3.75-3.68 (m, 1H), 3.12 (s, 2H), 2.87 (d, J=10.8 Hz, 2H), 2.69-2.59 (m, 2H), 2.39-2.32 (m, 1H), 2.30 (s, 3H), 2.22-2.10 (m, 3H), 1.65 (d, J=11.2 Hz, 2H), 1.43-1.33 (m, 2H), 1.33-1.21 (m, 2H), 1.21-1.10 (m, 2H), 0.96 (d, J=6.4 Hz, 3H)

### Step 9: N-[3-(2,6-dioxo-3-piperidyl)-1-methyl-indazol-6-yl]-2-[4-[(2S)-2-methyl-3-[3-methyl-4-(4,4,5,5-tetramethyl-1,3,2-dioxaborolan-2-yl)phenoxy]propyl]-1-piperidyl]acetamide

A mixture of 2-[4-[(2S)-3-(4-bromo-3-methyl-phenoxy)-2-methyl-propyl]-1-piperidyl]-N-[3-(2,6-dioxo-3-piperidyl)-1-methyl-indazol-6-yl]acetamide (524 mg, 838.98 mol, 1.0 equiv.), bis(pinacolato)diboron (426.10 mg, 1.68 mmol, 2.0 equiv.), KOAc (247.02 mg, 2.52 mmol, 3.0 equiv.), and Pd(dppf)Cl₂ (61.39 mg, 83.90 mol, 0.1 equiv.) in dioxane (10 mL) was degassed and purged with N₂ three times. The mixture was stirred at 90° C. for 12 hours under a N₂ atmosphere. Ethyl acetate (100 mL) and H₂O (50 mL) were added and the layers were separated. The aqueous layer was extracted with ethyl acetate (60 mL), dried over anhydrous sodium sulfate, filtered, and concentrated under reduced pressure to give a residue, which was purified by column chromatography (SiO₂, petroleum ether/ethyl acetate=1/1 to DCM/MeOH=10/1) to give N-[3-(2,6-dioxo-3-piperidyl)-1-methyl-indazol-6-yl]-2-[4-[(2S)-2-methyl-3-[3-methyl-4-(4,4,5,5-tetramethyl-1,3,2-dioxaborolan-2-yl)phenoxy]propyl]-1-piperidyl]acetamide (700 mg) as a yellow solid.

LC-MS (ESI): m/z=672.3 [M+H]⁺

### Step 10: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate

A mixture of N-[3-(2,6-dioxo-3-piperidyl)-1-methyl-indazol-6-yl]-2-[4-[(2S)-2-methyl-3-[3-methyl-4-(4,4,5,5-tetramethyl-1,3,2-dioxaborolan-2-yl)phenoxy]propyl]-1-piperidyl]acetamide (250 mg, 372.23 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (311.21 mg, 446.67 mol, 1.2 equiv.), H₂O (0.5 mL), KF (64.88 mg, 1.12 mmol, 26.16 μL, 3.0 equiv.), and Ad₂nBuP Pd G₃ (271.08 mg, 372.23 mol, 1.0 equiv.) in dioxane (10 mL) was degassed and purged with N₂ three times. The mixture was stirred at 115° C. for 2 hours in a microwave reactor. The reaction mixture was concentrated under reduced pressure to give a residue, which was purified by flash silica gel chromatography (0~100% ethyl acetate/petroleum ether) and reversed-phase HPLC to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (180 mg) as a white solid LC-MS (ESI): m/z=1161.5 [M+H]⁺

### Step 11: 6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid]

To a solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (180 mg, 154.97 mol, 1.0 equiv.) in DCM (1 mL) was added TFA (2 mL). The mixture was stirred at 40° C. for 0.5 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-[8-(1,3-benzothiazol-2-ylcarbamoyl)-3,4-dihydro-1H-2,7-naphthyridin-2-yl]-3-[4-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid] (63.55 mg) as a yellow solid.

LC-MS (ESI): m/z=975.3 [M+H]⁺

¹H NMR (400 MHz, DMSO-d₆) δ=12.35-12.28 (m, 1H), 10.88 (s, 1H), 8.57 (d, J=4.4 Hz, 1H), 8.0-8.02 (m, 2H), 7.82 (d, J=8.4 Hz, 1H), 7.64 (d, J=8.4 Hz, 1H), 7.58 (d, J=5.2 Hz, 1H), 7.50-7.46 (m, 2H), 7.36 (t, J=8.0, 1H), 7.24-7.16 (m, 1H), 7.06 (d, J=8.8 Hz, 1H), 6.94 (d, J=8.4 Hz, 1H), 6.81 (d, J=1.6 Hz, 1H), 6.76-6.70 (m, 1H), 5.24 (s, 2H), 4.36 -4.30 (m, 1H), 3.97 (t, J=5.2 Hz, 2H), 3.93 (s, 3H), 3.85-3.80 (m, 1H), 3.76 (d, J=7.2 Hz, 1H), 3.08 (t, J=5.2 Hz, 2H), 2.97-2.79 (m, 2H), 2.65-2.57 (m, 2H), 2.46-2.41 (m, 2H), 2.22-2.13 (m, 2H), 2.05 (s, 3H), 2.03-1.90 (m, 2H), 1.78-1.66 (m, 2H), 1.47-1.38 (m, 2H), 1.37-1.11 (m, 4H), 0.99 (d, J=6.4 Hz, 3H)

### Example 31: 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]ethyl]-4-piperidyl]propoxy]-2-methyl-phenyl]pyridine-2-carboxylic acid (Compound 150)

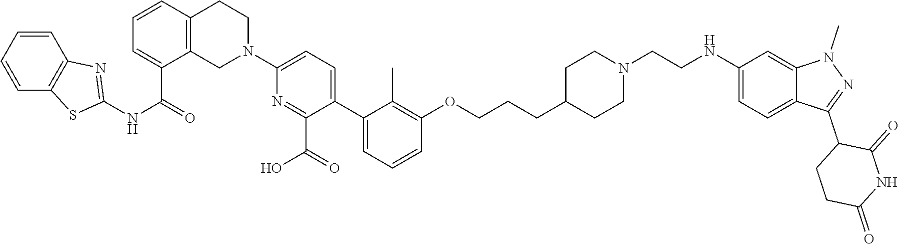 [SMILES: Cc1c(OCCCC2CCN(CCNc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### Step 1: 4-[3-(3-bromo-2-methyl-phenoxy)propyl]-1-(2,2-diethoxyethyl)piperidine

To a solution of 4-[3-(3-bromo-2-methyl-phenoxy)propyl]piperidine (0.35 g, 1.12 mmol, 1.0 equiv.) and 2-bromo-1,1-diethoxy-ethane (441.80 mg, 2.24 mmol, 337.25 μL, 2.0 equiv.) in DMF (8 mL) was added potassium carbonate (464.75 mg, 3.36 mmol, 3.0 equiv.). The mixture was stirred at 60° C. for 19 hours. The reaction mixture was quenched by the addition of H₂O (20 mL) and extracted with ethyl acetate (60 mL). The combined organic layers were washed with brine (60 mL), dried over sodium sulfate, filtered, and concentrated under reduced pressure to give a residue, which was purified by flash silica gel chromatography (0~17% ethyl acetate/petroleum ether) to give 4-[3-(3-bromo-2-methyl-phenoxy)propyl]-1-(2,2-diethoxyethyl)piperidine (310 mg) as a yellow oil.

LC-MS (ESI): m/z=429.9 [M+H]⁺

¹H NMR (400 MHz, DMSO-d₆) δ=7.13 (d, J=7.2 Hz, 1H), 7.08 (d, J=8.0 Hz, 1H), 6.95 (d, J=8.0 Hz, 1H), 4.55 (t, J=5.2 Hz, 1H), 3.96 (t, J=6.4 Hz, 2H), 3.57 (qd, J=7.2, 9.6 Hz, 2H), 3.45 (qd, J=7.2, 9.6 Hz, 2H), 2.85 (d, J=11.6 Hz, 2H), 2.36 (d, J=5.2 Hz, 2H), 2.22 (s, 3H), 1.96 (t, J=11.2 Hz, 2H), 1.79-1.68 (m, 2H), 1.61 (d, J=12.0 Hz, 2H), 1.38-1.30 (m, 2H), 1.25-1.19 (m, 1H), 1.10 (t, J=7.2 Hz, 8H).

### Step 2: tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate

A mixture of 4-[3-(3-bromo-2-methyl-phenoxy)propyl]-1-(2,2-diethoxyethyl)piperidine (250 mg, 583.56 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (393.21 mg, 641.92 mol, 1.1 equiv.), KF (101.71 mg, 1.75 mmol, 3.0 equiv.), and Ad₂nBuP Pd G₃ (85.00 mg, 116.71 mol, 0.2 equiv.) in dioxane (5 mL) and H₂O (0.5 mL) was degassed and purged with N₂ three times. The mixture was stirred at 100° C. for 16 hours under a N₂ atmosphere. The reaction mixture was quenched by the addition of H₂O (20 mL) and extracted with ethyl acetate (60 mL). The combined organic layers were washed with brine (60 mL), dried over sodium sulfate, filtered, and concentrated under reduced pressure to give a residue, which was purified by flash silica gel chromatography (0-55% ethyl acetate/petroleum ether) to give tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (330 mg) as a yellow solid LC-MS (ESI): m/z=834.4 [M+H]⁺

### Step 3: 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (330 mg, 395.65 mol, 1.0 equiv.) in DCM (1.5 mL) and TFA (1.5 mL) was stirred at 40° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (330 mg, crude) as a red oil.

LC-MS (ESI): m/z=704.2 [M+H]⁺

### Step 4: 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]ethyl]-4-piperidyl]propoxy]-2-methyl-phenyl]pyridine-2-carboxylic acid

A mixture of 3-(6-amino-1-methyl-indazol-3-yl)piperidine-2,6-dione (92.84 mg, 359.45 mol, 1.1 equiv.) and NaBH(OAc)₃ (207.77 mg, 980.33 mol, 3.0 equiv.) in DCM (50 mL) at 0° C. was stirred for 1 hour. To this mixture was added 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (230 mg, 326.78 mol, 1.0 equiv.). The resulting mixture was stirred at 0° C. for 1 hour. The reaction mixture was quenched by the addition of H₂O (50 mL) and extracted with DCM/MeOH (10/1) (150 mL). The combined organic layers were dried over sodium sulfate, filtered, and concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]ethyl]-4-piperidyl]propoxy]-2-methyl-phenyl]pyridine-2-carboxylic acid (59.69 mg) as an off-white solid.

LC-MS (ESI): m/z=946.3 [M+H]⁺

¹H NMR (400 MHz, DMSO-d₆) δ=10.82 (s, 1H), 8.01 (d, J=7.6 Hz, 1H), 7.78 (d, J=8.4 Hz, 1H), 7.62 (d, J=7.6 Hz, 1H), 7.51-7.31 (m, 6H), 7.08 (t, J=7.6 Hz, 1H), 6.89 (dd, J=8.8, 17.6 Hz, 2H), 6.65 (d, J=7.6 Hz, 1H), 6.55 (d, J=8.8 Hz, 1H), 6.40 (s, 1H), 6.00-5.79 (m, 1H), 4.96 (s, 2H), 4.19 (dd, J=5.2, 8.8 Hz, 1H), 4.00-3.90 (m, 4H), 3.81 (s, 3H), 3.27 (d, J=5.6 Hz, 4H), 3.02 (s, 2H), 2.72-2.69 (m, 2H), 2.59 (s, 2H), 2.25-2.10 (m, 4H), 1.90 (s, 3H), 1.78-1.62 (m, 4H), 1.34 (d, J=5.2 Hz, 3H), 1.24-1.09 (m, 2H).

### Example 32: 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinic acid (Compound 151)

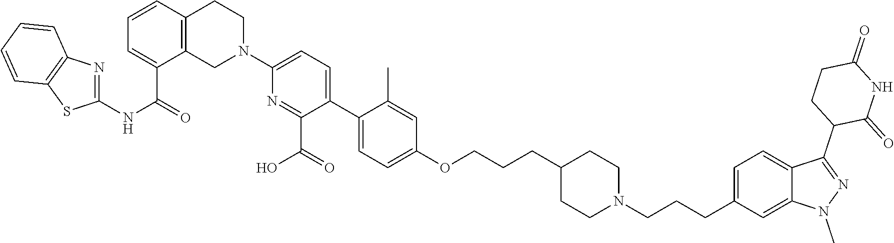 [SMILES: Cc1cc(OCCCC2CCN(CCCc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: 3-(2,6-bis(benzyloxy)pyridin-3-yl)-6-(3,3-diethoxyprop-1-yn-1-yl)-1-methyl-1H-indazole

A mixture of 3-(2,6-bis(benzyloxy)pyridin-3-yl)-6-bromo-1-methyl-1H-indazole (5.0 g, 9.99 mmol, 1 equiv.), 3,3-diethoxyprop-1-yne (1.54 g, 11.99 mmol, 1.72 mL, 1.2 equiv.), Pd(PPh₃)₂Cl₂ (701.35 mg, 999.23 mol, 0.1 equiv.), and CuI (380.61 mg, 2.00 mmol, 0.2 equiv.) in DIPEA (40 mL) and DMF (40 mL) was degassed and purged with N₂ three times. The mixture was stirred at 80° C. for 2 hours under a N₂ atmosphere. The mixture was concentrated under reduced pressure to give a residue, which was purified by flash silica gel chromatography (0~20% ethyl acetate/petroleum ether) to afford 3-(2,6-bis(benzyloxy)pyridin-3-yl)-6-(3,3-diethoxyprop-1-yn-1-yl)-1-methyl-1H-indazole (4.2 g) as a yellow oil.

LC-MS (ESI): m/z=548.2 [M+H]⁺

¹H NMR (400 MHz, DMSO-d₆) δ=7.91 (d, J=8.4 Hz, 1H), 7.86 (s, 1H), 7.67 (d, J=8.4 Hz, 1H), 7.49-7.46 (m, 2H), 7.42-7.27 (m, 9H), 7.06-7.02 (m, 1H), 5.56 (s, 1H), 5.44 (d, J=6.0 Hz, 4H), 4.07 (s, 3H), 3.76-3.69 (m, 2H), 3.64-3.58 (m, 2H), 1.19 (t, J=7.2 Hz, 6H).

### Step 2: 3-(6-(3,3-diethoxypropyl)-1-methyl-1H-indazol-3-yl)piperidine-2,6-dione

A mixture of 3-(2,6-bis(benzyloxy)pyridin-3-yl)-6-(3,3-diethoxyprop-1-yn-1-yl)-1-methyl-1H-indazole (4.2 g, 7.67 mmol, 1.0 equiv.), Pd on carbon (10 wt %, 2.04 g, 1.92 mmol, 0.25 equiv.), and Pd(OH)₂ on carbon (20 wt %, 2.15 g, 3.07 mmol, 0.4 equiv.) in EtOH (40 mL) and THE (40 mL) was degassed and purged with H₂ three times. The mixture was stirred at 25° C. for 16 hours under H₂ atmosphere. The mixture was filtered and concentrated under reduced pressure to give a residue, which was purified by flash silica gel chromatography (0~40% ethyl acetate/petroleum ether) to afford 3-(6-(3,3-diethoxypropyl)-1-methyl-1H-indazol-3-yl)piperidine-2,6-dione (2.1 g) as a yellow oil.

LC-MS (ESI): m/z=374.1 [M+H]⁺

### Step 3: 3-(3-(2,6-dioxopiperidin-3-yl)-1-methyl-1H-indazol-6-yl)propanal

A mixture of 3-(6-(3,3-diethoxypropyl)-1-methyl-1H-indazol-3-yl)piperidine-2,6-dione (2.1 g, 5.62 mmol, 1.0 equiv.) in HCOOH (21 mL) was stirred at 80° C. for 1 hour. The mixture was concentrated under reduced pressure to afford 3-(3-(2,6-dioxopiperidin-3-yl)-1-methyl-1H-indazol-6-yl)propanal (2.1 g, crude) as a black oil.

LC-MS (ESI): m/z=300.1 [M+H]⁺

### Step 4: 3-(6-(3-(4-(3-(4-bromo-3-methylphenoxy)propyl)piperidin-1-yl)propyl)-1-methyl-1H-indazol-3-yl)piperidine-2,6-dione

A mixture of 3-(3-(2,6-dioxopiperidin-3-yl)-1-methyl-1H-indazol-6-yl)propanal (200 mg, 668.17 mol, 1.0 equiv.) and 4-(3-(4-bromo-3-methylphenoxy)propyl)piperidine (250.36 mg, 801.81 mol, 1.2 equiv.) in DCM (5 mL) was stirred at 25° C. for 2.5 hours. To this mixture was added NaBH₃CN (125.97 mg, 2.00 mmol, 3.0 equiv.). The resulting mixture was stirred at 25° C. for 0.5 hours. The mixture was concentrated under reduced pressure to give a residue, which was purified by reversed-phase HPLC to afford 3-(6-(3-(4-(3-(4-bromo-3-methylphenoxy)propyl)piperidin-1-yl)propyl)-1-methyl-1H-indazol-3-yl)piperidine-2,6-dione (200 mg) as a white solid.

LC-MS (ESI): m/z=596.9 [M+H]⁺

### Step 5: tert-butyl 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinate

A mixture of 3-(6-(3-(4-(3-(4-bromo-3-methylphenoxy)propyl)piperidin-1-yl)propyl)-1-methyl-1H-indazol-3-yl)piperidine-2,6-dione (100 mg, 167.91 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (123.42 mg, 201.49 mol, 1.2 equiv.), KF (29.26 mg, 503.72 mol, 3.0 equiv.), and Ad₂nBuP Pd G₃ (36.68 mg, 50.37 mol, 0.3 equiv.) in dioxane (3 mL) and H₂O (0.3 mL) was degassed and purged with N₂ three times. The mixture was stirred at 100° C. for 2 hours under a N₂ atmosphere. The mixture was concentrated under reduced pressure to give a residue, which was purified by flash silica gel chromatography (0~10% methanol/dichloromethane) to afford tert-butyl 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinate (100 mg) as a yellow solid.

LC-MS (ESI): m/z=1001.4 [M+H]⁺

### Step 6: 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinate (100 mg, 99.88 mol, 1.0 equiv.) in TFA (1 mL) and DCM (1 mL) was stirred at 25° C. for 2 hours. The mixture was concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(4-(3-(1-(3-(3-(2,6-dioxopiperidin-3-yl)-1-methyl-1H-indazol-6-yl)propyl)piperidin-4-yl)propoxy)-2-methylphenyl)picolinic acid (47.18 mg) as a yellow solid.

LC-MS (ESI): m/z=945.3 [M+H]⁺

¹H NMR (400 MHz, DMSO-d₆) δ=10.88 (s, 1H), 8.14 (s, 1H), 8.02 (d, J=7.6 Hz, 1H), 7.78 (d, J=8.0 Hz, 1H), 7.61 (dd, J=2.8, 8.4 Hz, 2H), 7.47-7.39 (m, 4H), 7.39-7.32 (m, 2H), 6.99 (d, J=8.0 Hz, 1H), 6.92 (d, J=8.0 Hz, 2H), 6.77 (d, J=2.4 Hz, 1H), 6.69 (dd, J=2.0, 8.4 Hz, 1H), 4.96 (s, 2H), 4.33 (dd, J=4.8, 9.6 Hz, 1H), 3.95 (s, 3H), 3.94-3.88 (m, 4H), 3.11-3.07 (m, 2H), 3.01 (t, J=5.2 Hz, 2H), 2.73 (t, J=7.6 Hz, 2H), 2.67-2.57 (m, 4H), 2.39-2.26 (m, 3H), 2.19-2.14 (m, 1H), 2.02 (s, 3H), 1.93-1.87 (m, 2H), 1.71 (d, J=9.6 Hz, 4H), 1.36-1.29 (m, 3H), 1.25-1.17 (m, 2H)

### Example 33: 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylic acid (Compound 149)

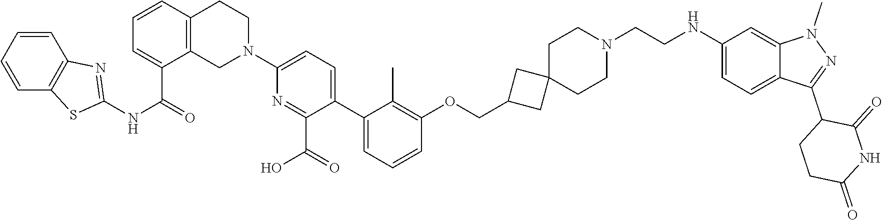 [SMILES: Cc1c(OCC2CC3(CCN(CCNc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### Step 1: tert-butyl 2-[(3-bromo-2-methyl-phenoxy)methyl]-7-azaspiro[3.5]nonane-7-carboxylate

To a solution of 3-bromo-2-methyl-phenol (1 g, 5.35 mmol, 1.0 equiv.), tert-butyl 2-(hydroxymethyl)-7-azaspiro[3.5]nonane-7-carboxylate (1.64 g, 6.42 mmol, 1.2 equiv.), and 2-(tributyl-phosphanylidene)acetonitrile (1.94 g, 8.02 mmol, 1.5 equiv.) were taken up into a microwave tube in toluene (10 mL). The sealed tube was heated at 130° C. for 2 hours in a microwave reactor. The reaction mixture was concentrated under reduced pressure to give a residue, which was purified by reversed-phase HPLC to give tert-butyl 2-[(3-bromo-2-methyl-phenoxy)methyl]-7-azaspiro[3.5]nonane-7-carboxylate (800 mg) as a yellow oil.

LC-MS (ESI): m/z=368.4 [M+H−56]⁺.

### Step 2: 2-[(3-bromo-2-methyl-phenoxy)methyl]-7-azaspiro[3.5]nonane

To a solution of tert-butyl 2-[(3-bromo-2-methyl-phenoxy)methyl]-7-azaspiro[3.5]nonane-7-carboxylate (800 mg, 1.89 mmol, 1.0 equiv.) in dioxane (2 mL) was added HCl/dioxane (2 M, 15 mL, 15.91 equiv.). The mixture was stirred at 25° C. for 2 hours. The reaction mixture was concentrated under reduced pressure to give 2-[(3-bromo-2-methyl-phenoxy)methyl]-7-azaspiro[3.5]nonane (650 mg) as a yellow solid.

LC-MS (ESI): m/z=324.0 [M+H]⁺

¹H NMR (400 MHz, DMSO-d₆) δ=7.17-7.13 (m, 1H), 7.11-7.05 (m, 1H), 6.95 (d, J=7.6 Hz, 1H), 3.95 (d, J=6.0 Hz, 2H), 2.98-2.86 (m, 4H), 2.73-2.65 (m, 1H), 2.23 (s, 3H), 1.98-1.92 (m, 2H), 1.81-1.77 (m, 2H), 1.74-1.61 (m, 5H)

### Step 3: 3-[6-[3-[2-[(3-bromo-2-methyl-phenoxy)methyl]-7-azaspiro[3.5]nonan-7-yl]propyl]-1-methyl-indazol-3-yl]piperidine-2,6-dione

To a solution of 2-[(3-bromo-2-methyl-phenoxy)methyl]-7-azaspiro[3.5]nonane (250 mg, 693.07 mol, 1.0 equiv., HCl) and 3-(3-(2,6-dioxopiperidin-3-yl)-1-methyl-1H-indazol-6-yl)propanal (311.18 mg, 831.68 mol, 1.2 equiv.) in DCM (10 mL) was added NaBH(OAc)₃ (440.67 mg, 2.08 mmol, 3.0 equiv.). The mixture was stirred at 25° C. for 1 hour. The reaction was diluted with H₂O (30 mL) and washed with DCM (90 mL). The combined organic layers were washed with brine (50 mL), dried over anhydrous sodium sulfate, filtered, and concentrated under reduced pressure to give a residue, which was purified by flash silica gel chromatography (0-15% dichloromethane/methanol) to give 3-[6-[3-[2-[(3-bromo-2-methyl-phenoxy)methyl]-7-azaspiro[3.5]nonan-7-yl]propyl]-1-methyl-indazol-3-yl]piperidine-2,6-dione (95 mg) as a yellow solid.

LC-MS (ESI): m/z=609.1 [M+H]⁺

### Step 4: tert-butyl 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylate

A mixture of 3-[6-[3-[2-[(3-bromo-2-methyl-phenoxy)methyl]-7-azaspiro[3.5]nonan-7-yl]propyl]-1-methyl-indazol-3-yl]piperidine-2,6-dione (95 mg, 129.78 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (95.39 mg, 155.73 mol, 1.2 equiv.), KF (22.62 mg, 389.33 mol, 3.0 equiv.), and Ad₂nBuP Pd G3 (9.45 mg, 12.98 mol, 0.1 equiv.) in dioxane (2 mL) and H₂O (0.2 mL) was degassed and purged with N₂ three times. The mixture was stirred at 100° C. for 3 hours under a N₂ atmosphere. The reaction mixture was concentrated under reduced pressure to give a residue, which was purified by flash silica gel chromatography (0~10% dichloromethane/methanol) to give tert-butyl 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylate (70 mg) as a yellow solid

LC-MS (ESI): m/z=1013.3 [M+H]⁺

### Step 5: 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylate (70 mg, 69.08 mol, 1.0 equiv.) in TFA (2 mL) and DCM (1 mL) was stirred at 25° C. for 8 hours. The reaction mixture was concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[3-[[7-[3-[3-(2,6-dioxo-3-piperidyl)-1-methyl-indazol-6-yl]propyl]-7-azaspiro[3.5]nonan-2-yl]methoxy]-2-methyl-phenyl]pyridine-2-carboxylic acid (35.50 mg) as a off-white solid.

LC-MS (ESI): m/z=957.3 [M+H]⁺

¹H NMR (400 MHz, DMSO-d₆) δ=10.87 (s, 1H), 8.01 (d, J=8.0 Hz, 1H), 7.77 (d, J=8.0 Hz, 1H), 7.60 (dd, J=8.0, 12.8 Hz, 2H), 7.49-7.41 (m, 2H), 7.41-7.30 (m, 4H), 7.09-7.01 (m, 1H), 6.97 (d, J=8.4 Hz, 1H), 6.92-6.79 (m, 2H), 6.64 (d, J=7.6 Hz, 1H), 4.96 (s, 2H), 4.32 (dd, J=5.2, 9.6 Hz, 1H), 3.94 (s, 3H), 3.92-3.87 (m, 4H), 3.01 (t, J=5.6 Hz, 2H), 2.71-2.56 (m, 6H), 2.42-2.27 (m, 6H), 2.16 (dd, J=5.4, 13.6 Hz, 1H), 1.89 (s, 3H), 1.88-1.76 (m, 4H), 1.67-1.56 (m, 4H), 1.48 (s, 2H).

### Example 34: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinic acid (Compound 152)

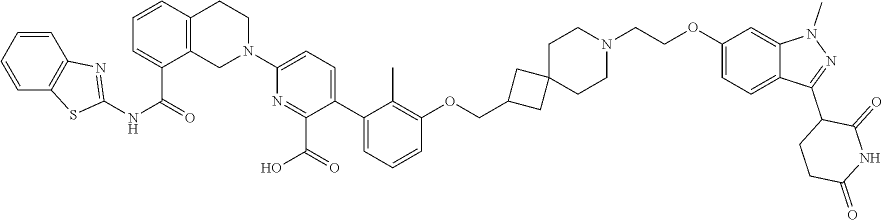 [SMILES: Cc1c(OCC2CC3(CCN(CCOc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### Step 1: 3-(2,6-dibenzyloxy-3-pyridyl)-1-methyl-indazol-6-ol

A mixture of 6-bromo-3-(2,6-dibenzyloxy-3-pyridyl)-1-methyl-indazole (2.5 g, 5.00 mmol, 1.0 equiv.), t-BuONa (1.44 g, 14.99 mmol, 3.0 equiv.), tBuBrettPhos Pd G₃ (426.88 mg, 499.61 mol, 0.1 equiv.) and H₂O (270.02 mg, 14.99 mmol, 270.02 μL, 3.0 equiv.) in THF (30 mL) was degassed and purged with N₂ three times. The mixture was stirred at 60° C. for 12 hours under N₂ atmosphere. The reaction mixture was partitioned between ethyl acetate (300 mL) and water (200 mL). The organic phase was separated, washed with bine (400 mL), dried over sodium sulfate, filtered and concentrated under reduced pressure to give a residue, which was purified by flash silica gel chromatography (0-43% ethyl acetate/petroleum ether) to give 3-(2,6-dibenzyloxy-3-pyridyl)-1-methyl-indazol-6-ol (2.0 g) as a yellow oil.

LC-MS (ESI): m/z=438.2 [M+H]⁺

### Step 2: 3-(2,6-dibenzyloxy-3-pyridyl)-6-(2,2-diethoxyethoxy)-1-methyl-indazole

To a solution of 3-(2,6-dibenzyloxy-3-pyridyl)-1-methyl-indazol-6-ol (2.0 g, 4.57 mmol, 1.0 equiv.) and 2-bromo-1,1-diethoxy-ethane (1.80 g, 9.14 mmol, 1.38 mL, 2.0 equiv.) in DMF (20 mL) was added TEA (1.38 g, 13.71 mmol, 1.9 mL, 3.0 equiv.) and KI (758.89 mg, 4.57 mmol, 1.0 equiv.). The mixture was stirred at 60° C. for 12 hours. The reaction mixture was partitioned between ethyl acetate (200 mL) and water (100 mL). The organic phase was separated, washed with bine (300 mL), dried over sodium sulfate, filtered, and concentrated under reduced pressure to give a residue, which was purified by flash silica gel chromatography (0~20% ethyl acetate/petroleum ether) to give 3-(2,6-dibenzyloxy-3-pyridyl)-6-(2,2-diethoxyethoxy)-1-methyl-indazole (1.9 g) as a yellow oil.

LC-MS (ESI): m/z=554.2 [M+H]⁺

### Step 3: 3-[6-(2,2-diethoxyethoxy)-1-methyl-indazol-3-yl]piperidine-2,6-dione

A mixture of 3-(2,6-dibenzyloxy-3-pyridyl)-6-(2,2-diethoxyethoxy)-1-methyl-indazole (1.9 g, 3.43 mmol, 1.0 equiv.), Pd on carbon (10 wt %, 803.46 mg, 754.99 mol, 0.22 equiv.), Pd(OH)₂ on carbon (20 wt %, 795.21 mg, 1.13 mmol, 0.33 equiv.) in THE (30 mL) and EtOH (30 mL) was degassed and purged with H₂ three times. The mixture was stirred at 40° C. for 12 hours under H₂ (15 psi) atmosphere. The mixture was filtered, and the filter liquid was concentrated under reduced pressure to give a residue, which was purified by flash silica gel chromatography (0-80% ethyl acetate/petroleum ether) to give 3-[6-(2,2-diethoxyethoxy)-1-methyl-indazol-3-yl]piperidine-2,6-dione (1.0 g) as a white solid.

LC-MS (ESI): m/z=376.2 [M+H]⁺

### Step 4: 2-[3-(2,6-dioxo-3-piperidyl)-1-methyl-indazol-6-yl]oxyacetaldehyde

A solution of 3-[6-(2,2-diethoxyethoxy)-1-methyl-indazol-3-yl]piperidine-2,6-dione (200 mg, 141.18 mol, 1.0 equiv.) in HCOOH (3 mL) was stirred at 40° C. for 1 hour. The reaction mixture was concentrated under reduced pressure to give 2-[3-(2,6-dioxo-3-piperidyl)-1-methyl-indazol-6-yl]oxyacetaldehyde (150 mg) as a yellow oil.

LC-MS (ESI): m/z=320.1 [M+H]⁺

### Step 5: 3-(6-(2-(2-((3-bromo-2-methylphenoxy)methyl)-7-azaspiro[3.5]nonan-7-yl)ethoxy)-1-methyl-1H-indazol-3-yl)piperidine-2,6-dione

A solution of 2-((3-bromo-2-methylphenoxy)methyl)-7-azaspiro[3.5]nonane (102.24 mg, 315.30 mol, 1.0 equiv.) and NaBH₃CN (59.44 mg, 945.91 mol, 3.0 equiv.) in DCM (1 mL) was stirred at 25° C. for 1 hour followed by the addition of 2-[3-(2,6-dioxo-3-piperidyl)-1-methyl-indazol-6-yl]oxyacetaldehyde (95 mg, 315.30 mol, 1.0 equiv.). The mixture was stirred at 25° C. for 1 hour. The reaction mixture was filtered and concentrated under reduced pressure to give a residue, which was purified by flash silica gel chromatography (0-7% methanol/dichloromethane) to give 3-(6-(2-(2-((3-bromo-2-methylphenoxy)methyl)-7-azaspiro[3.5]nonan-7-yl)ethoxy)-1-methyl-1H-indazol-3-yl)piperidine-2,6-dione (130 mg) as a yellow solid.

¹H NMR (400 MHz, DMSO-d₆) δ=10.86 (s, 1H), 7.56 (d, J=8.8 Hz, 1H), 7.15 (d, J=7.6 Hz, 1H), 7.11-7.07 (m, 2H), 6.96 (d, J=7.6 Hz, 1H), 6.74 (d, J=9.2 Hz, 1H), 4.29 (dd, J=5.2, 9.2 Hz, 1H), 4.25-4.09 (m, 2H), 3.95 (d, J=6.4 Hz, 2H), 3.93 (s, 3H), 2.75-2.64 (m, 3H), 2.64-2.58 (m, 2H), 2.36-2.28 (m, 2H), 2.24 (s, 3H), 2.23-2.10 (m, 2H), 1.94-1.85 (m, 2H), 1.65 (t, J=8.4 Hz, 4H), 1.56-1.50 (m, 2H), 1.33-1.15 (m, 2H).

### Step 6: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinate

A mixture of 3-(6-(2-(2-((3-bromo-2-methylphenoxy)methyl)-7-azaspiro[3.5]nonan-7-yl)ethoxy)-1-methyl-1H-indazol-3-yl)piperidine-2,6-dione (100 mg, 164.05 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (120.59 mg, 196.87 mol, 1.2 equiv.), Ad₂nBuP Pd G₃ (11.95 mg, 16.41 mol, 0.1 equiv.), and KF (28.59 mg, 492.16 mol, 3.0 equiv.) in dioxane (2 mL) and H₂O (0.2 mL) was degassed and purged with N₂ three times. The mixture was stirred at 100° C. for 2 hours under a N₂ atmosphere. The reaction mixture was filtered and concentrated under reduced pressure to give a residue, which was purified by flash silica gel chromatography (0-7% methanol/dichloromethane) to give tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinate (120 mg) as a yellow solid.

LC-MS (ESI): m/z=1015.3 [M+H]⁺

### Step 7: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinate (120 mg, 118.20 mol, 1.0 equiv.) in TFA (2 mL) and DCM (1 mL) was stirred at 40° C. for 1 hour. The reaction mixture was filtered and concentrated under reduced pressure to give a residue, which was purified by reversed-phase HPLC to give 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)oxy)ethyl)-7-azaspiro[3.5]nonan-2-yl)methoxy)-2-methylphenyl)picolinic acid (41.17 mg) as a white solid.

LC-MS (ESI): m/z=959.3 [M+H]⁺

¹H NMR (400 MHz, DMSO-d₆) δ=10.85 (s, 1H), 8.02 (d, J=8.0 Hz, 1H), 7.78 (d, J=8.4 Hz, 1H), 7.62 (d, J=7.6 Hz, 1H), 7.54 (d, J=8.8 Hz, 1H), 7.50-7.40 (m, 3H), 7.39-7.32 (m, 2H), 7.12-7.04 (t, J=8.0 Hz, 2H), 6.94 (d, J=9.2 Hz, 1H), 6.87 (d, J=8.4 Hz, 1H), 6.72 (dd, J=1.6, 8.8 Hz, 1H), 6.64 (d, J=7.6 Hz, 1H), 4.98 (s, 2H), 4.29 (dd, J=4.8, 9.2 Hz, 1H), 4.14 (t, J=5.6 Hz, 2H), 3.92 (s, 7H), 3.02 (t, J=6.0 Hz, 2H), 2.72-2.67 (m, 3H), 2.65-2.59 (m, 2H), 2.47-2.44 (m, 1H), 2.43-2.25 (m, 4H), 2.18-2.13 (m, 1H), 1.93-1.86 (m, 5H), 1.69-1.60 (m, 4H), 1.52 (s, 2H).

### Example 35: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid (Compound 154)

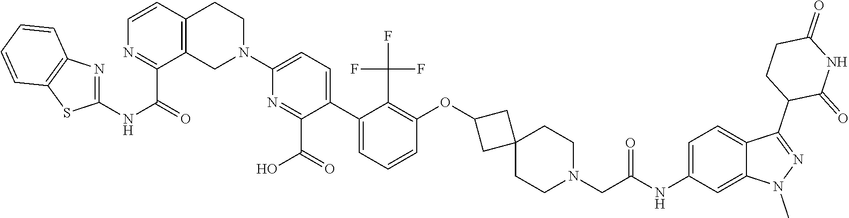 [SMILES: Cn1nc(C2CCC(=O)NC2=O)c2ccc(NC(=O)CN3CCC4(CC3)CC(Oc3cccc(-c5ccc(N6CCc7ccnc(C(=O)Nc8nc9ccccc9s8)c7C6)nc5C(=O)O)c3C(F)(F)F)C4)cc21]

### Step 1: tert-butyl 2-[3-bromo-2-(trifluoromethyl)phenoxy]-7-azaspiro[3.5]nonane-7-carboxylate

A mixture of tert-butyl 2-hydroxy-7-azaspiro[3.5]nonane-7-carboxylate (1 g, 4.14 mmol, 1.0 equiv.), 3-bromo-2-(trifluoromethyl)phenol (1.20 g, 4.97 mmol, 1.2 equiv.), and 2-(tributyl-phosphanylidene)acetonitrile (1.50 g, 6.22 mmol, 1.5 equiv.) in toluene (10 mL) was degassed and purged with N₂ three times. The mixture was stirred at 120° C. for 12 hours under a N₂ atmosphere. The reaction mixture was concentrated under reduced pressure to give a residue, which was purified by reversed-phase HPLC to give tert-butyl 2-[3-bromo-2-(trifluoromethyl)phenoxy]-7-azaspiro[3.5]nonane-7-carboxylate (1.5 g) as a brown oil.

LC-MS (ESI): m/z=409.9 [M+H−56]⁺.

### Step 2: 2-[3-bromo-2-(trifluoromethyl)phenoxy]-7-azaspiro[3.5]nonane

To a solution of tert-butyl 2-[3-bromo-2-(trifluoromethyl)phenoxy]-7-azaspiro[3.5]nonane-7-carboxylate (1.5 g, 3.23 mmol, 1.0 equiv.) in dioxane (5 mL) was added HCl/dioxane (2 M, 20 mL, 12.38 equiv.). The mixture was stirred at 25° C. for 2 hours. The reaction mixture was filtered to give 2-[3-bromo-2-(trifluoromethyl)phenoxy]-7-azaspiro[3.5]nonane (1.25 g, crude) as a white solid.

¹H NMR (400 MHz, DMSO-d₆) δ=7.50-7.43 (m, 1H), 7.41-7.36 (m, 1H), 7.09 (d, J=8.4 Hz, 1H), 4.89 (t, J=6.0 Hz, 1H), 3.01-2.89 (m, 4H), 2.50-2.43 (m, 2H), 1.89-1.82 (m, 2H), 1.81-1.70 (m, 4H)

### Step 3: ethyl 2-[2-[3-bromo-2-(trifluoromethyl)phenoxy]-7-azaspiro[3.5]nonan-7-yl]acetate

To a solution of 2-[3-bromo-2-(trifluoromethyl)phenoxy]-7-azaspiro[3.5]nonane (500 mg, 1.37 mmol, 1.0 equiv.) and ethyl 2-bromoacetate (229.27 mg, 1.37 mmol, 151.94 μL, 1.0 equiv.) in DMF (5 mL) was added potassium carbonate (569.22 mg, 4.12 mmol, 3.0 equiv.). The mixture was stirred at 40° C. for 1 hour. The reaction mixture was diluted with H₂O (50 mL) and extracted with EtOAc (150 mL). The combined organic layers were washed with H₂O (150 mL), dried over sodium sulfate, filtered, and concentrated under reduced pressure to give ethyl 2-[2-[3-bromo-2-(trifluoromethyl)phenoxy]-7-azaspiro[3.5]nonan-7-yl]acetate (550 mg, crude) as a yellow oil.

LC-MS (ESI): m/z=451.8 [M+H]⁺

### Step 4: 2-[2-[3-bromo-2-(trifluoromethyl)phenoxy]-7-azaspiro[3.5]nonan-7-yl]acetic acid

To a solution of ethyl 2-[2-[3-bromo-2-(trifluoromethyl)phenoxy]-7-azaspiro[3.5]nonan-7-yl]acetate (550 mg, 1.22 mmol, 1.0 equiv.) in THE (4 mL) and H₂O (1 mL) was added LiOH·H₂O (153.77 mg, 3.66 mmol, 3.0 equiv.). The mixture was stirred at 40° C. for 1 hour. The reaction mixture was diluted with H₂O (30 mL) and extracted with DCM (90 mL). The combined organic layers were washed with H₂O (100 mL), dried over sodium sulfate, filtered, and concentrated under reduced pressure to give 2-[2-[3-bromo-2-(trifluoromethyl)phenoxy]-7-azaspiro[3.5]nonan-7-yl]acetic acid (600 mg, crude) as a white solid, which was used into next step without further purification.

¹H NMR (400 MHz, DMSO-d₆) δ=7.49-7.42 (m, 1H), 7.40-7.35 (m, 1H), 7.09 (d, J=8.4 Hz, 1H), 4.92-4.84 (m, 1H), 3.59 (t, J=6.4 Hz, 1H), 3.34 (s, 2H), 3.07-2.87 (m, 4H), 2.49-2.41 (m, 2H), 1.84-1.72 (m, 6H).

### Step 5: 2-[2-[3-bromo-2-(trifluoromethyl)phenoxy]-7-azaspiro[3.5]nonan-7-yl]-N-[3-(2,6-dioxo-3-piperidyl)-1-methyl-indazol-6-yl]acetamide

To a solution of 2-[2-[3-bromo-2-(trifluoromethyl)phenoxy]-7-azaspiro[3.5]nonan-7-yl]acetic acid (150 mg, 355.25 mol, 1.0 equiv.) and 3-(6-amino-1-methyl-indazol-3-yl)piperidine-2,6-dione (110.10 mg, 426.30 mol, 1.2 equiv.) in pyridine (2 mL) was added EDCI (88.53 mg, 461.83 mol, 1.3 equiv.). The mixture was stirred at 25° C. for 1 hour. The reaction mixture was concentrated under reduced pressure to remove pyridine. The mixture was diluted with H₂O (30 mL) and extracted with EtOAc (90 mL). The combined organic layers were washed with H₂O (100 mL), dried over sodium sulfate, filtered, and concentrated under reduced pressure to give a residue, which was purified by flash silica gel chromatography (0-100% ethyl acetate/petroleum ether) to give 2-[2-[3-bromo-2-(trifluoromethyl)phenoxy]-7-azaspiro[3.5]nonan-7-yl]-N-[3-(2,6-dioxo-3-piperidyl)-1-methyl-indazol-6-yl]acetamide (180 mg) as a light red solid.

LC-MS (ESI): m/z=663.9 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate

A mixture of tert-butyl 6-[8-[1,3-benzothiazol-2-yl(2-trimethylsilylethoxymethyl)carbamoyl]-3,4-dihydro-1H-2,7-naphthyridin-2-yl]-3-bromo-pyridine-2-carboxylate (500 mg, 717.64 mol, 1.0 equiv.), bis(pinacolato)diboron (546.71 mg, 2.15 mmol, 3.0 equiv.), Pd(dppf)Cl₂ (105.02 mg, 143.53 mol, 0.2 equiv.), and KOAc (211.29 mg, 2.15 mmol, 3.0 equiv.) in dioxane (6 mL) was degassed and purged with N₂ three times. The mixture was stirred at 90° C. for 4 hours under a N₂ atmosphere. The reaction mixture was diluted with water (100 mL) and extracted with ethyl acetate (300 mL). The combined organic layers were washed with brine (200 mL), dried over sodium sulfate, filtered, and concentrated under reduced pressure to give a residue, which was purified by flash silica gel chromatography (0~10% ethyl acetate/petroleum ether) to afford tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (500 mg) as a yellow oil.

LC-MS (ESI): m/z=744.2 [M+H]⁺

### Step 7: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate

A mixture of 2-[2-[3-bromo-2-(trifluoromethyl)phenoxy]-7-azaspiro[3.5]nonan-7-yl]-N-[3-(2,6-dioxo-3-piperidyl)-1-methyl-indazol-6-yl]acetamide (90 mg, 135.85 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (121.25 mg, 163.02 mol, 1.2 equiv.), Ad₂nBuP Pd G3 (49.47 mg, 67.92 mol, 0.5 equiv.), and KF (23.68 mg, 407.55 mol, 3.0 equiv.) in dioxane (1 mL) and H₂O (0.1 mL) was degassed and purged with N₂ three times. The mixture was stirred at 80° C. for 12 hours under a N₂ atmosphere. The reaction mixture was concentrated under reduced pressure to give a residue, which was purified by flash silica gel chromatography (0~100% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate (80 mg) as a yellow solid.

LC-MS (ESI): m/z=957.3 [M+H]⁺

### Step 8: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate (80 mg, 66.70 mol, 1.0 equiv.) in TFA (2 mL) and DCM (1 mL) was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid (21.74 mg) as a yellow solid.

LC-MS (ESI): m/z=1013.2 [M+H]⁺

¹H NMR (400 MHz, DMSO-d₆) δ=10.88 (s, 1H), 9.84 (s, 1H), 8.56 (d, J=4.4 Hz, 1H), 8.09-7.99 (m, 2H), 7.81 (d, J=8.0 Hz, 1H), 7.63 (d, J=8.4 Hz, 1H), 7.58 (d, J=4.4 Hz, 1H), 7.52-7.42 (m, 3H), 7.39-7.33 (m, 1H), 7.25-7.20 (m, 1H), 7.03 (dd, J=8.4, 15.2 Hz, 2H), 6.70 (d, J=7.6 Hz, 1H), 5.34-5.17 (m, 2H), 4.94-4.85 (m, 1H), 4.32 (dd, J=5.2, 9.6 Hz, 1H), 4.04-3.95 (m, 2H), 3.92 (s, 3H), 3.13 (s, 2H), 3.07 (t, J=5.2 Hz, 2H), 2.73-2.55 (m, 3H), 2.47-2.42 (m, 4H), 2.40-2.27 (m, 2H), 2.21-2.14 m, 1H), 1.85-1.77 (m, 2H), 1.73-1.64 (m, 4H).

### Example 36: 6-[8-(1,3-benzothiazol-2-ylcarbamoyl)-3,4-dihydro-1H-isoquinolin-2-yl]-3-[4-[3-[1-[2-[3-(2,6-dioxo-3-piperidyl)-1-methyl-indazol-6-yl]oxyethyl]-4-piperidyl]propoxy]-2-methyl-phenyl]pyridine-2-carboxylic acid (Compound 178)

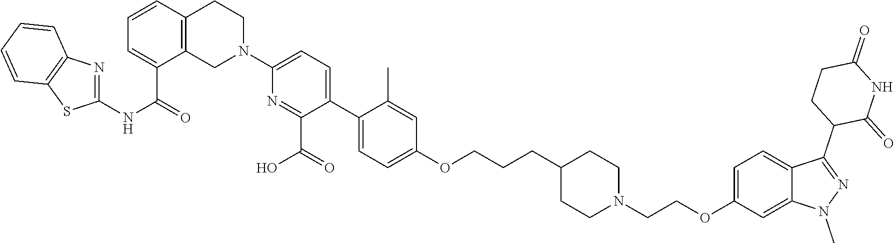 [SMILES: Cc1cc(OCCCC2CCN(CCOc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: tert-butyl 4-(3-(4-bromo-3-methylphenoxy)propyl)piperidine-1-carboxylate

A mixture of tert-butyl 4-(3-bromopropyl)piperidine-1-carboxylate (982.41 mg, 3.21 mmol, 1.2 equiv.), 4-bromo-3-methylphenol (500 mg, 2.67 mmol, 1 equiv.), and potassium carbonate (1.11 g, 8.02 mmol, 3 equiv.) in DMF (20 mL) was stirred at 60° C. for 2 hours. The reaction mixture was concentrated under reduced pressure to give a residue, which was diluted with H₂O (20 mL) and extracted with DCM (99 mL). The combined organic layers were washed with H₂O (60 mL), dried over anhydrous sodium sulfate, filtered, and concentrated under reduced pressure to give a residue, which was purified by reversed-phase HPLC to give tert-butyl 4-(3-(4-bromo-3-methylphenoxy)propyl)piperidine-1-carboxylate (600 mg) as a yellow oil.

LC-MS (ESI): m/z=312.0 [M−100+H]⁺.

¹H NMR (400 MHz, DMSO-d₆) δ=7.41 (d, J=8.8 Hz, 1H), 6.94 (d, J=2.8 Hz, 1H), 6.70 (dd, J=2.8, 8.8 Hz, 1H), 3.91 (t, J=6.4 Hz, 4H), 2.29 (s, 3H), 1.74-1.60 (m, 4H), 1.40-1.37 (m, 10H), 1.37-1.20 (m, 4H), 1.07-0.92 (m, 2H).

### Step 2: tert-butyl 6-(8-(benzo[d]thiazol-2-ylcarbamoyl)-3,4-dihydroisoquinolin-2(1H)-yl)-3-(4-(3-(1-(tert-butoxycarbonyl)piperidin-4-yl)propoxy)-2-methylphenyl)picolinate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (534.77 mg, 873.02 mol, 1.2 equiv.), tert-butyl 4-(3-(4-bromo-3-methylphenoxy)propyl)piperidine-1-carboxylate (300 mg, 727.52 mol, 1 equiv.), KF (126.81 mg, 2.18 mmol, 3 equiv.), and Ad₂nBup Pd G₃ (105.97 mg, 145.50 mol, 0.2 equiv.) in dioxane (8 mL) and H₂O (0.8 mL) was degassed and purged with N₂ three times. The mixture was stirred at 100° C. for 2 hours under a N₂ atmosphere. The reaction mixture was filtered and concentrated under reduced pressure to give a residue, which was purified by flash silica gel chromatography (0~30% ethyl acetate/petroleum ether) to give tert-butyl 6-(8-(benzo[d]thiazol-2-ylcarbamoyl)-3,4-dihydroisoquinolin-2(1H)-yl)-3-(4-(3-(1-(tert-butoxycarbonyl)piperidin-4-yl)propoxy)-2-methylphenyl)picolinate (500 mg) as a yellow solid.

LC-MS (ESI): m/z=818.3 [M+H]⁺

### Step 3: 6-(8-(benzo[d]thiazol-2-ylcarbamoyl)-3,4-dihydroisoquinolin-2(1H)-yl)-3-(2-methyl-4-(3-(piperidin-4-yl)propoxy)phenyl)picolinic acid

A solution of tert-butyl 6-(8-(benzo[d]thiazol-2-ylcarbamoyl)-3,4-dihydroisoquinolin-2(1H)-yl)-3-(4-(3-(1-(tert-butoxycarbonyl)piperidin-4-yl)propoxy)-2-methylphenyl)picolinate (280 mg, 342.28 mol, 1 equiv.) in TFA (0.8 mL) and DCM (2.4 mL) was stirred at 40° C. for 16 hours. The reaction mixture was filtered and concentrated under reduced pressure to give 6-(8-(benzo[d]thiazol-2-ylcarbamoyl)-3,4-dihydroisoquinolin-2(1H)-yl)-3-(2-methyl-4-(3-(piperidin-4-yl)propoxy)phenyl)picolinic acid (250 mg, crude) as a yellow oil.

LC-MS (ESI): m/z=662.3 [M+H]⁺

### Step 4: 6-[8-(1,3-benzothiazol-2-ylcarbamoyl)-3,4-dihydro-1H-isoquinolin-2-yl]-3-[4-[3-[1-[2-[3-(2,6-dioxo-3-piperidyl)-1-methyl-indazol-6-yl]oxyethyl]-4-piperidyl]propoxy]-2-methyl-phenyl]pyridine-2-carboxylic acid

To a mixture of 6-(8-(benzo[d]thiazol-2-ylcarbamoyl)-3,4-dihydroisoquinolin-2(1H)-yl)-3-(2-methyl-4-(3-(piperidin-4-yl)propoxy)phenyl)picolinic acid (46.52 mg, 59.74 mol, 1.2 equiv.) and NaBH₃CN (9.39 mg, 149.35 mol, 3 equiv.) in DCM (1.5 mL) was added DIPEA (0.1 mL). The mixture was stirred at 0° C. for 0.5 hours. To this mixture was added a solution of 2-[3-(2,6-dioxo-3-piperidyl)-1-methyl-indazol-6-yl]oxyacetaldehyde (20 mg, 49.78 mol, 1 equiv.) in DCM (0.5 mL). The mixture was stirred at 0° C. for 1 hour. The reaction mixture was filtered and concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[4-[3-[1-[2-[3-(2,6-dioxo-3-piperidyl)-1-methyl-indazol-6-yl]oxyethyl]-4-piperidyl]propoxy]-2-methyl-phenyl]pyridine-2-carboxylic acid (21.40 mg) as a white solid.

LC-MS (ESI): m/z=947.3 [M+H]⁺

¹H NMR (400 MHz, DMSO-d₆) δ=10.86 (s, 1H), 8.18 (s, 1H), 8.02 (d, J=7.6 Hz, 1H), 7.82-7.76 (m, 1H), 7.65-7.59 (m, 1H), 7.55 (d, J=8.8 Hz, 1H), 7.50-7.30 (m, 5H), 7.09 (d, J=2.0 Hz, 1H), 6.97-6.86 (m, 2H), 6.82-6.64 (m, 3H), 4.97 (s, 2H), 4.29 (dd, J=5.2, 9.2 Hz, 1H), 4.15 (t, J=5.6 Hz, 2H), 3.96-3.87 (m, 7H), 3.02 (t, J=5.6 Hz, 2H), 2.96 (d, J=11.6 Hz, 2H), 2.73 (t, J=5.2 Hz, 2H), 2.68-2.59 (m, 2H), 2.39-2.25 (m, 2H), 2.20-2.13 (m, 1H), 2.08-1.99 (m, 5H), 1.75-1.64 (m, 4H), 1.38-1.30 (m, 2H), 1.20-1.12 (m, 2H).

### Example 37: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)ethyl)piperidin-4-yl)propoxy)-2-methylphenyl)picolinic acid (Compound 179)

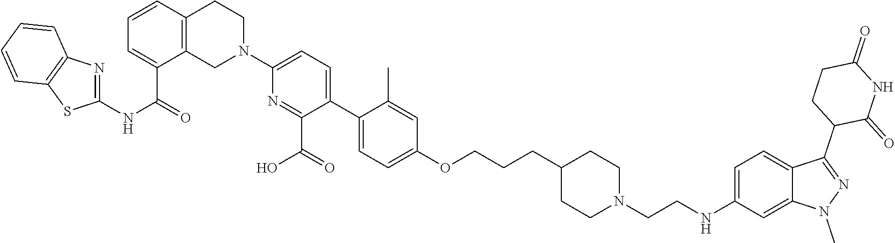 [SMILES: Cc1cc(OCCCC2CCN(CCNc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: 4-(3-(4-bromo-3-methylphenoxy)propyl)-1-(2,2-diethoxyethyl)piperidine

To a solution of 4-(3-(4-bromo-3-methylphenoxy)propyl)piperidine (320 mg, 1.02 mmol, 1 equiv.) and 2-bromo-1,1-diethoxyethane (222.16 mg, 1.13 mmol, 169.59 μL, 1.1 equiv.) in DMF (2 mL) was added potassium carbonate (424.91 mg, 3.07 mmol, 3 equiv.). The mixture was stirred at 80° C. for 10 hours. The reaction mixture was concentrated under reduced pressure to give a residue, which was purified by flash silica gel chromatography (0~60% ethyl acetate/petroleum ether) to give 4-(3-(4-bromo-3-methylphenoxy)propyl)-1-(2,2-diethoxyethyl)piperidine (360 mg) as a yellow oil.

LC-MS (ESI): m/z=428.1 [M+H]⁺

### Step 2: tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate

To a mixture of 4-(3-(4-bromo-3-methylphenoxy)propyl)-1-(2,2-diethoxyethyl)piperidine (360 mg, 840.33 mol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (566.22 mg, 924.37 mol, 1.1 equiv.) in dioxane (5 mL) and H₂O (0.5 mL) was added Ad₂nBup Pd G₃ (61.20 mg, 84.03 mol, 0.1 equiv.) and KF (146.46 mg, 2.52 mmol, 3 equiv.). The mixture was stirred at 100° C. for 3 hours. The reaction mixture was concentrated under reduced pressure to give a residue, which was purified by flash silica gel chromatography (0~50% ethyl acetate/petroleum ether) to give tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (400 mg) as a yellow solid.

LC-MS (ESI): m/z=834.3 [M+H]⁺

### Step 3: 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid

A solution of tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (400 mg, 479.57 mol, 1 equiv.) in DCM (1 mL) and TFA (1 mL) was stirred at 40° C. for 2 hours. The reaction mixture was concentrated under reduced pressure to give 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (350 mg) as a yellow oil.

LC-MS (ESI): m/z=704.2 [M+H]⁺

### Step 4: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)ethyl)piperidin-4-yl)propoxy)-2-methylphenyl)picolinic acid

A mixture of 3-(6-amino-1-methyl-indazol-3-yl)piperidine-2,6-dione (220.2 mg, 852.5 mol, 2 equiv.) and 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (300 mg, 426.2 mol, 1 equiv.) in DCM (5 mL) was stirred at 25° C. for 2 hours followed by the addition of NaBH₃CN (80.4 mg, 1278.7 mol, 3 equiv.) at 25° C. The resulting mixture was stirred at 25° C. for 0.5 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)ethyl)piperidin-4-yl)propoxy)-2-methylphenyl)picolinic acid (15.23 mg) as a yellow solid.

LC-MS (ESI): m/z=946.3 [M+H]⁺

¹H NMR (400 MHz, DMSO-d₆) δ=10.82 (s, 1H), 8.03 (d, J=7.6 Hz, 1H), 7.79 (d, J=8.0 Hz, 1H), 7.63 (d, J=7.6 Hz, 1H), 7.50-7.32 (m, 6H), 6.93 (dd, J=8.8, 12.4 Hz, 2H), 6.78 (s, 1H), 6.70 (dd, J=2.0, 8.4 Hz, 1H), 6.56 (d, J=8.8 Hz, 1H), 6.40 (s, 1H), 5.87-5.67 (m, 1H), 4.97 (s, 2H), 4.19 (dd, J=5.2, 8.8 Hz, 1H), 3.98-3.89 (m, 4H), 3.82 (s, 3H), 3.24-3.23 (m, 2H), 3.04-2.96 (m, 4H), 2.69-2.57 (m, 4H), 2.29-2.23 (m, 1H), 2.20-2.13 (m, 1H), 2.11-2.00 (m, 5H), 1.74-1.62 (m, 4H), 1.39-1.28 (m, 3H), 1.21-1.13 (m, 2H).

### Example 38: 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]ethyl]-7-azaspiro[3.5]nonan-2-yl]methoxy]-2-methyl-phenyl]pyridine-2-carboxylic acid (Compound 153)

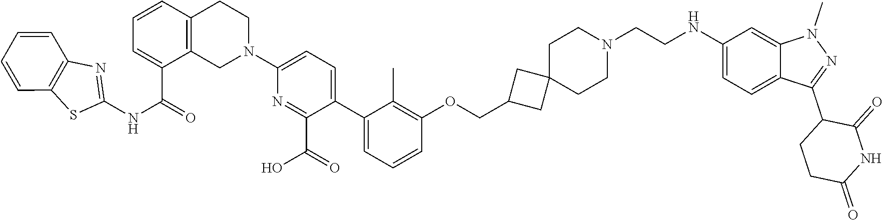 [SMILES: Cc1c(OCC2CC3(CCN(CCNc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### Step 1: 2-[(3-bromo-2-methyl-phenoxy)methyl]-7-(2,2-diethoxyethyl)-7-azaspiro[3.5]nonane

To a solution of 2-[(3-bromo-2-methyl-phenoxy)methyl]-7-azaspiro[3.5]nonane (300 mg, 831.68 mol, 1.0 equiv., HCl) in DMF (10 mL) was added potassium carbonate (344.83 mg, 2.50 mmol, 3.0 equiv.) and 2-bromo-1,1-diethoxy-ethane (327.80 mg, 1.66 mmol, 250.23 L, 2.0 equiv.). The mixture was stirred at 60° C. for 12 hours. The reaction mixture was concentrated under reduced pressure to give a residue, which was purified by flash silica gel chromatography (0-10% ethyl acetate/petroleum ether) to give 2-[(3-bromo-2-methyl-phenoxy)methyl]-7-(2,2-diethoxyethyl)-7-azaspiro[3.5]nonane (330 mg) as a yellow oil.

LC-MS (ESI): m/z=440.2 [M+H]⁺

¹H NMR (400 MHz, DMSO-d₆) δ=7.17-7.13 (d, J=8.0 Hz, 1H), 7.09 (t, J=8.0 Hz, 1H), 6.96 (d, J=8.0 Hz, 1H), 4.55 (t, J=5.2 Hz, 1H), 3.94 (d, J=6.0 Hz, 2H), 3.62-3.53 (m, 2H), 3.49-3.43 (m, 2H), 2.75-2.59 (m, 2H), 2.42-2.33 (m, 5H), 2.24 (s, 3H), 1.90-1.83 (m, 2H), 1.64-1.56 (m, 4H), 1.47 (t, J=5.2 Hz, 2H), 1.10 (t, J=7.2 Hz, 6H).

### Step 2: tert-butyl 6-[8-(1,3-benzothiazol-2-ylcarbamoyl)-3,4-dihydro-1H-isoquinolin-2-yl]-3-[3-[[7-(2,2-diethoxyethyl)-7-azaspiro[3.5]nonan-2-yl]methoxy]-2-methyl-phenyl]pyridine-2-carboxylate

A mixture of 2-[(3-bromo-2-methyl-phenoxy)methyl]-7-(2,2-diethoxyethyl)-7-azaspiro[3.5]nonane (220 mg, 499.53 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (367.18 mg, 599.44 mol, 1.2 equiv.), KF (87.06 mg, 1.50 mmol, 3.0 equiv.), H₂O (0.8 mL), and Ad₂nBup Pd G₃ (72.76 mg, 99.91 mol, 0.2 equiv.) in dioxane (8 mL) was degassed and purged with N₂ three times. The mixture was stirred at 100° C. for 2 hours under a N₂ atmosphere. The reaction mixture was concentrated under reduced pressure to give a residue, which was purified by flash silica gel chromatography (0-50% ethyl acetate/petroleum ether) give tert-butyl 6-[8-(1,3-benzothiazol-2-ylcarbamoyl)-3,4-dihydro-1H-isoquinolin-2-yl]-3-[3-[[7-(2,2-diethoxyethyl)-7-azaspiro[3.5]nonan-2-yl]methoxy]-2-methyl-phenyl]pyridine-2-carboxylate (300 mg) as a yellow solid.

LC-MS (ESI): m/z=846.4 [M+H]⁺

### Step 3: 6-[8-(1,3-benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[[7-(2,2-diethoxyethyl)-7-azaspiro[3.5]nonan-2-yl]methoxy]-2-methyl-phenyl]pyridine-2-carboxylate (300 mg, 354.57 mol, 1.0 equiv.) in DCM (3 mL) was added TFA (6.14 g, 53.85 mmol, 4 mL). The mixture was stirred at 40° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid (250 mg, crude) as a yellow solid.

LC-MS (ESI): m/z=716.3 [M+H]⁺

### Step 4: 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]ethyl]-7-azaspiro[3.5]nonan-2-yl]methoxy]-2-methyl-phenyl]pyridine-2-carboxylic acid

To a solution of 3-(6-amino-1-methyl-indazol-3-yl)piperidine-2,6-dione (123.52 mg, 419.08 mol, 1.2 equiv., HCl) in DCM (30 mL) was added NaBH(OAc)₃ (296.07 mg, 1.40 mmol, 4.0 equiv.). The pH was adjusted to pH=6. To this mixture was added 6-[8-(1,3-benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid (250 mg, 349.23 mol, 1.0 equiv.) in DCM (10 mL) dropwise at 0° C. The mixture was stirred at 0° C. for 1 hour. The reaction mixture was diluted with water (20 mL) and extracted with DCM (100 mL). The combined organic layers were dried over sodium sulfate, filtered, and concentrated under reduced pressure to give a residue, which was purified by prep-HPLC to give 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-6-yl]amino]ethyl]-7-azaspiro[3.5]nonan-2-yl]methoxy]-2-methyl-phenyl]pyridine-2-carboxylic acid (22.70 mg) as a pink solid.

LC-MS (ESI): m/z=958.3 [M+H]⁺

¹H NMR (400 MHz, DMSO-d₆) δ=10.82 (s, 1H), 8.03 (d, J=7.6 Hz, 1H), 7.79 (d, J=8.0 Hz, 1H), 7.63 (d, J=8.0 Hz, 1H), 7.50-7.42 (m, 3H), 7.41-7.31 (m, 3H), 7.08 (t, J=8.0 Hz, 1H), 6.94 (d, J=8.8 Hz, 1H), 6.87 (d, J=8.4 Hz, 1H), 6.65 (d, J=8.0 Hz, 1H), 6.54 (d, J=8.8 Hz, 1H), 6.39 (s, 1H), 5.80-5.75 (m, 1H), 4.98 (s, 2H), 4.20-4.17 (m, 1H), 3.92 (s, 4H), 3.82 (s, 3H), 3.20 (s, 2H), 3.03 (s, 2H), 2.68 (d, J=1.6 Hz, 2H), 2.61-2.58 (m, 4H), 2.39-2.36 (m, 1H), 2.31-2.20 (m, 2H), 2.18-2.12 (m, 1H), 1.99-1.84 (m, 6H), 1.71-1.62 (m, 4H), 1.52 (s, 2H).

### Example 39: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid (Compound 157)

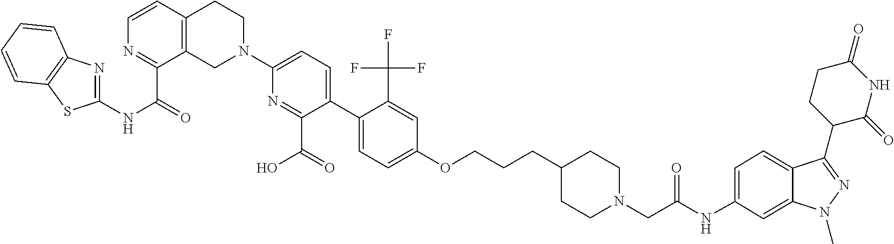 [SMILES: Cn1nc(C2CCC(=O)NC2=O)c2ccc(NC(=O)CN3CCC(CCCOc4ccc(-c5ccc(N6CCc7ccnc(C(=O)Nc8nc9ccccc9s8)c7C6)nc5C(=O)O)c(C(F)(F)F)c4)CC3)cc21]

### Step 1: tert-butyl 4-[3-[4-bromo-3-(trifluoromethyl)phenoxy]propyl]piperidine-1-carboxylate

To a solution of tert-butyl 4-(3-bromopropyl)piperidine-1-carboxylate (1.0 g, 3.27 mmol, 1.0 equiv.) and 4-bromo-3-(trifluoromethyl)phenol (944.38 mg, 3.92 mmol, 1.2 equiv.) in DMF (10 mL) was added potassium carbonate (1.35 g, 9.80 mmol, 3.0 equiv.). The mixture was stirred at 60° C. for 1 hour. The reaction mixture was diluted with water (100 mL) and extracted with ethyl acetate (300 mL). The combined organic layers were washed with brine (200 mL), dried over sodium sulfate, filtered, and concentrated under reduced pressure to give a residue, which was purified by prep-HPLC to give tert-butyl 4-[3-[4-bromo-3-(trifluoromethyl)phenoxy]propyl]piperidine-1-carboxylate (1.3 g) as a yellow solid.

LC-MS (ESI): m/z=410.0 [M−56+H]⁺.

### Step 2: 4-[3-[4-bromo-3-(trifluoromethyl)phenoxy]propyl]piperidine

A solution of tert-butyl 4-[3-[4-bromo-3-(trifluoromethyl)phenoxy]propyl]piperidine-1-carboxylate (1.3 g, 2.79 mmol, 1.0 equiv.) in HCl/dioxane (10 mL) was stirred at 25° C. for 1 hour. The mixture was concentrated under reduced pressure to give 4-[3-[4-bromo-3-(trifluoromethyl)phenoxy]propyl]piperidine (1.1 g) as a white solid.

LC-MS (ESI): m/z=368.0 [M+H]⁺

¹H NMR (400 MHz, DMSO-d₆) δ=9.05-8.53 (m, 2H), 7.76 (d, J=8.8 Hz, 1H), 7.31 (d, J=2.8 Hz, 1H), 7.18 (dd, J=2.8, 8.8 Hz, 1H), 4.04 (t, J=6.4 Hz, 2H), 3.22 (d, J=12.4 Hz, 2H), 2.80 (q, J=11.2 Hz, 2H), 1.88-1.68 (m, 4H), 1.54-1.58 (m, 1H), 1.41-1.21 (m, 4H)

### Step 3: ethyl 2-[4-[3-[4-bromo-3-(trifluoromethyl)phenoxy]propyl]-1-piperidyl]acetate

To a solution of 4-[3-[4-bromo-3-(trifluoromethyl)phenoxy]propyl]piperidine (900 mg, 2.46 mmol, 1.0 equiv.) in DMF (10 mL) was added TEA (746.04 mg, 7.37 mmol, 1.03 mL, 3.0 equiv.) and ethyl 2-bromoacetate (451.46 mg, 2.70 mmol, 299.18 μL, 1.1 equiv.). The mixture was stirred at 25° C. for 0.15 hours. The reaction mixture was concentrated under reduced pressure to give a residue, which was purified by flash silica gel chromatography (0-20% ethyl acetate/petroleum ether) to give ethyl 2-[4-[3-[4-bromo-3-(trifluoromethyl)phenoxy]propyl]-1-piperidyl]acetate (990 mg) as a yellow oil

LC-MS (ESI): m/z=452.2 [M+H]⁺ 1H NMR (400 MHz, DMSO-d₆) δ=7.75 (d, J=8.8 Hz, 1H), 7.31 (d, J=2.8 Hz, 1H), 7.18 (dd, J=2.8, 8.8 Hz, 1H), 4.08-4.01 (m, 4H), 3.15 (s, 2H), 2.79 (d, J=11.2 Hz, 2H), 2.13 (t, J=8.0 Hz, 2H), 1.76-1.68 (m, 2H), 1.62 (d, J=12.4 Hz, 2H), 1.36-1.29 (m, 2H), 1.22-1.12 (m, 6H)

### Step 4: 2-[4-[3-[4-bromo-3-(trifluoromethyl)phenoxy]propyl]-1-piperidyl]acetic acid

To a solution of ethyl 2-[4-[3-[4-bromo-3-(trifluoromethyl)phenoxy]propyl]-1-piperidyl]acetate (500 mg, 1.11 mmol, 1.0 equiv.) in THE (4 mL) and H₂O (0.8 mL) was added LiOH·H₂O (139.17 mg, 3.32 mmol, 3.0 equiv.). The mixture was stirred at 40° C. for 2 hours. The pH of the mixture was adjusted to pH=5-6 with 1M HCl. The mixture was then extracted with ethyl acetate (30 mL). The combined organic layers were washed with brine (20 mL), dried over sodium sulfate, filtered, and concentrated under reduced pressure to give 2-[4-[3-[4-bromo-3-(trifluoromethyl)phenoxy]propyl]-1-piperidyl]acetic acid (450 mg) as a yellow solid.

LC-MS (ESI): m/z=425.9 [M+H]⁺

### Step 5: 2-[4-[3-[4-bromo-3-(trifluoromethyl)phenoxy]propyl]-1-piperidyl]-N-[3-(2,6-dioxo-3-piperidyl)-1-methyl-indazol-6-yl]acetamide

To a solution of 2-[4-[3-[4-bromo-3-(trifluoromethyl)phenoxy]propyl]-1-piperidyl]acetic acid (300 mg, 707.13 mol, 1.0 equiv.) and 3-(6-amino-1-methyl-indazol-3-yl)piperidine-2,6-dione (219.16 mg, 848.55 mol, 1.2 equiv.) in pyridine (1 mL) was added EDCI (203.34 mg, 1.06 mmol, 1.5 equiv.). The mixture was stirred at 25° C. for 2 hours. The reaction mixture was concentrated under reduced pressure to give a residue, which was purified by flash silica gel chromatography (0~10% MeOH/DCM) to give 2-[4-[3-[4-bromo-3-(trifluoromethyl)phenoxy]propyl]-1-piperidyl]-N-[3-(2,6-dioxo-3-piperidyl)-1-methyl-indazol-6-yl]acetamide (230 mg) as a yellow solid.

LC-MS (ESI): m/z=664.1 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate

A mixture of 2-[4-[3-[4-bromo-3-(trifluoromethyl)phenoxy]propyl]-1-piperidyl]-N-[3-(2,6-dioxo-3-piperidyl)-1-methyl-indazol-6-yl]acetamide (90 mg, 135.44 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (159.06 mg, 162.53 mol, 1.2 equiv.), Ad₂nBuP Pd G₃ (49.32 mg, 67.72 mol, 0.5 equiv.), and KF (23.61 mg, 406.31 mol, 3.0 equiv.) in dioxane (2 mL) and H₂O (0.1 mL) was degassed and purged with N₂ three times. The mixture was stirred at 80° C. for 2 hours under a N₂ atmosphere. The reaction mixture was diluted with water (20 mL) and extracted with DCM (60 mL). The combined organic layers were washed with brine (40 mL), dried over sodium sulfate, filtered, and concentrated under reduced pressure to give a residue, which was purified by flash silica gel chromatography (0~10% MeOH/DCM) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (96 mg) as a yellow solid.

LC-MS (ESI): m/z=601.5 [M+H]/2⁺.

### Step 7: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (80 mg, 66.59 mol, 1.0 equiv.) in DCM (1.5 mL) and TFA (1.5 mL) was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid (33.19 mg) as a yellow solid.

LC-MS (ESI): m/z=1015.2 [M+H]⁺

¹H NMR (400 MHz, DMSO-d₆) δ=12.47-12.20 (m, 1H), 10.89 (s, 1H), 8.58 (d, J=4.8 Hz, 1H), 8.10-8.00 (m, 2H), 7.82 (d, J=8.0 Hz, 1H), 7.67 (d, J=7.6 Hz, 1H), 7.60 (d, J=4.4 Hz, 1H), 7.54-7.45 (m, 2H), 7.37 (t, J=7.6 Hz, 1H), 7.25-7.15 (m, 4H), 7.10 (d, J=9.2 Hz, 1H), 5.35-5.21 (m, 2H), 4.34 (dd, J=4.8, 9.6 Hz, 1H), 4.12-4.06 (m, 2H), 4.04-3.98 (m, 2H), 3.94 (s, 3H), 3.14-2.79 (m, 6H), 2.69-2.61 (m, 2H), 2.41-2.29 (m, 2H), 2.27-2.07 (m, 2H), 1.91-1.70 (m, 4H), 1.54-1.30 (m, 5H).

### Example 40: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid (Compound 158)

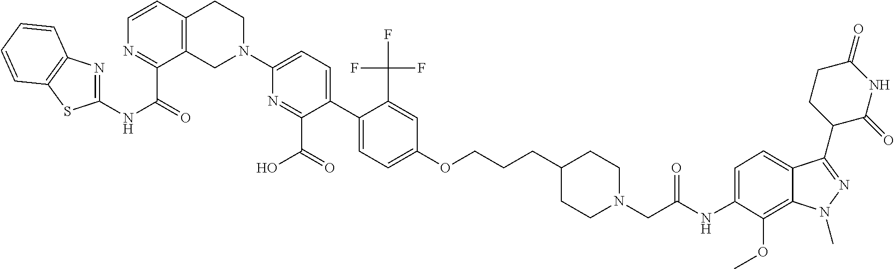 [SMILES: COc1c(NC(=O)CN2CCC(CCCOc3ccc(-c4ccc(N5CCc6ccnc(C(=O)Nc7nc8ccccc8s7)c6C5)nc4C(=O)O)c(C(F)(F)F)c3)CC2)ccc2c(C3CCC(=O)NC3=O)nn(C)c12]

### Step 1: 2-[4-[3-[4-bromo-3-(trifluoromethyl)phenoxy]propyl]-1-piperidyl]-N-[3-(2,6-dioxo-3-piperidyl)-7-methoxy-1-methyl-indazol-6-yl]acetamide

To a solution of 2-[4-[3-[4-bromo-3-(trifluoromethyl)phenoxy]propyl]-1-piperidyl]acetic acid (200 mg, 471.42 mol, 1.0 equiv.) and 3-(6-amino-7-methoxy-1-methyl-indazol-3-yl)piperidine-2,6-dione (149.50 mg, 518.56 mol, 1.1 equiv.) in pyridine (5 mL) was added EDCI (135.56 mg, 707.13 mol, 1.5 equiv.). The mixture was stirred at 40° C. for 3 hours. The reaction mixture was concentrated under reduced pressure to give a residue, which was purified by flash silica gel chromatography (0~10% MeOH/DCM) to give 2-[4-[3-[4-bromo-3-(trifluoromethyl)phenoxy]propyl]-1-piperidyl]-N-[3-(2,6-dioxo-3-piperidyl)-7-methoxy-1-methyl-indazol-6-yl]acetamide (290 mg) as a red solid.

LC-MS (ESI): m/z=695.1 [M+H]⁺

¹H NMR (400 MHz, DMSO-d₆) δ=10.89 (s, 1H), 9.84 (s, 1H), 8.00 (d, J=2.8 Hz, 1H), 7.76 (d, J=8.8 Hz, 1H), 7.45 (d, J=8.8 Hz, 1H), 7.32 (d, J=3.2 Hz, 1H), 7.19 (dd, J=2.8, 8.8 Hz, 1H), 4.34 (dd, J=5.2, 10.0 Hz, 1H), 4.14 (s, 3H), 4.09-4.04 (m, 2H), 3.89 (s, 3H), 3.15 (s, 2H), 2.94 (d, J=2.0 Hz, 2H), 2.71-2.61 (m, 2H), 2.22 (d, J=16.4 Hz, 2H), 1.81-1.73 (m, 4H), 1.44-1.23 (m, 7H).

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate

A mixture of 2-[4-[3-[4-bromo-3-(trifluoromethyl)phenoxy]propyl]-1-piperidyl]-N-[3-(2,6-dioxo-3-piperidyl)-7-methoxy-1-methyl-indazol-6-yl]acetamide (110 mg, 158.38 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (159.19 mg, 158.38 mol, 1.0 equiv.), KF (27.61 mg, 475.14 mol, 3.0 equiv.), and Ad₂nBuP Pd G₃ (57.67 mg, 79.19 mol, 0.5 equiv.) in dioxane (2 mL) and H₂O (0.2 mL) was degassed and purged with N₂ three times. The mixture was stirred at 80° C. for 2 hours under a N₂ atmosphere. The reaction mixture was concentrated under reduced pressure to give a residue, which was purified by prep-TLC (SiO₂, DCM:MeOH=15:1) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate (160 mg) as a yellow solid.

LC-MS (ESI): m/z=1231.8 [M+H]⁺

### Step 3: 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate (160 mg, 129.93 mol, 1.0 equiv.) in DCM (1 mL) and TFA (1 mL) was stirred at 40° C. for 2 hours. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid (24.67 mg) as an off-white solid.

LC-MS (ESI): m/z=1045.3 [M+H]⁺

¹H NMR (400 MHz, DMSO-d₆) δ=12.62-12.42 (m, 1H), 12.31 (s, 1H), 10.89 (s, 1H), 9.85 (s 1H), 8.57 (d, J=4.8 Hz, 1H), 8.05 (d, J=8.0, 1H), 8.03-7.93 (m, 1H), 7.82 (d, J=8.0 Hz, 1H), 7.59 (d, J=4.8 Hz, 1H), 7.53-7.42 (m, 3H), 7.36 (t, J=7.6 Hz, 1H), 7.25-7.15 (m, 3H), 7.09 (d, J=8.8 Hz, 1H), 5.37-5.10 (m, 2H), 4.34 (dd, J=5.2, 10.0 Hz, 1H), 4.14 (s, 3H), 4.08 (t, J=6.4 Hz, 2H), 4.04-3.95 (m, 2H), 3.90 (s, 3H), 3.16 (d, J=2.4 Hz, 2H), 3.08 (t, J=5.2 Hz, 2H), 3.03-2.90 (m, 2H), 2.70-2.60 (m, 2H), 2.35-2.14 (m, 4H), 1.83-1.75 (m, 4H), 1.48-1.40 (m, 2H), 1.39-1.24 (m, 3H).

### Example 41: 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid (Compound 159)

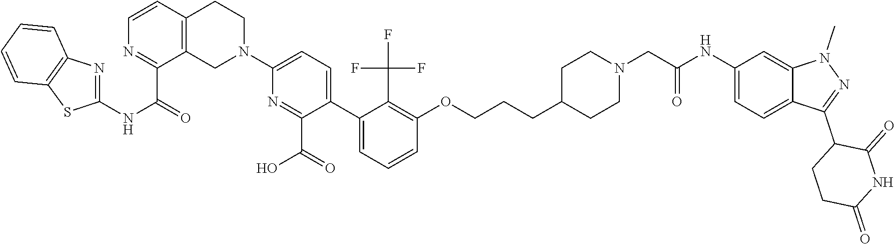 [SMILES: Cn1nc(C2CCC(=O)NC2=O)c2ccc(NC(=O)CN3CCC(CCCOc4cccc(-c5ccc(N6CCc7ccnc(C(=O)Nc8nc9ccccc9s8)c7C6)nc5C(=O)O)c4C(F)(F)F)CC3)cc21]

### Step 1: tert-butyl 4-[3-[3-bromo-2-(trifluoromethyl)phenoxy]propyl]piperidine-1-carboxylate

To a solution of tert-butyl 4-(3-bromopropyl)piperidine-1-carboxylate (1.0 g, 3.27 mmol, 1.0 equiv.) and 3-bromo-2-(trifluoromethyl)phenol (944.38 mg, 3.92 mmol, 1.2 equiv.) in DMF (10 mL) was added potassium carbonate (1.35 g, 9.80 mmol, 3.0 equiv.). The mixture was stirred at 60° C. for 2 hours. The reaction mixture was diluted with water (100 mL) and extracted with ethyl acetate (300 mL). The combined organic layers were washed with brine (200 mL), dried over sodium sulfate, filtered, and concentrated under reduced pressure to give a residue, which was purified by prep-HPLC to give tert-butyl 4-[3-[3-bromo-2-(trifluoromethyl)phenoxy]propyl]piperidine-1-carboxylate (1.3 g) as a yellow oil.

LC-MS (ESI): m/z=410.0 [M−56+H]⁺.

### Step 2: 4-[3-[3-bromo-2-(trifluoromethyl)phenoxy]propyl]piperidine

A solution of tert-butyl 4-[3-[3-bromo-2-(trifluoromethyl)phenoxy]propyl]piperidine-1-carboxylate (1.3 g, 2.79 mmol, 1.0 equiv.) in HCl/dioxane (10 mL) was stirred at 25° C. for 1 hour. The mixture was concentrated under reduced pressure to give 4-[3-[3-bromo-2-(trifluoromethyl)phenoxy]propyl]piperidine (900 mg) as a white solid.

LC-MS (ESI): m/z=366.0 [M+H]⁺

¹H NMR (400 MHz, DMSO-d₆) δ=9.01-8.46 (m, 2H), 7.52-7.44 (m, 1H), 7.38 (d, J=8.0 Hz, 1H), 7.28 (d, J=8.4 Hz, 1H), 4.10 (t, J=6.0 Hz, 2H), 3.22 (d, J=12.4 Hz, 2H), 2.86-2.75 (m, 2H), 1.84-1.69 (m, 4H), 1.61-1.48 (m, 1H), 1.41-1.22 (m, 4H).

### Step 3: ethyl 2-[4-[3-[3-bromo-2-(trifluoromethyl)phenoxy]propyl]-1-piperidyl]acetate

To a solution of 4-[3-[3-bromo-2-(trifluoromethyl)phenoxy]propyl]piperidine (900 mg, 2.46 mmol, 1.0 equiv.) in acetone (10 mL) was added TEA (746.04 mg, 7.37 mmol, 1.03 mL, 3.0 equiv.) and ethyl 2-bromoacetate (451.46 mg, 2.70 mmol, 299.18 μL, 1.1 equiv.). The mixture was stirred at 25° C. for 0.2 hours. The reaction mixture was concentrated under reduced pressure to give a residue, which was purified by flash silica gel chromatography (0~20% ethyl acetate/petroleum ether) to give ethyl 2-[4-[3-[3-bromo-2-(trifluoromethyl)phenoxy]propyl]-1-piperidyl]acetate (1.0 g) as a yellow oil.

LC-MS (ESI): m/z=452.1 [M+H]⁺

### Step 4: 2-[4-[3-[3-bromo-2-(trifluoromethyl)phenoxy]propyl]-1-piperidyl]acetic acid

To a solution of ethyl 2-[4-[3-[3-bromo-2-(trifluoromethyl)phenoxy]propyl]-1-piperidyl]acetate (500 mg, 1.11 mmol, 1.0 equiv.) in THE (4 mL) and H₂O (1 mL) was added LiOH·H₂O (139.17 mg, 3.32 mmol, 3.0 equiv.). The mixture was stirred at 40° C. for 2 hours. The pH of the mixture was adjusted to 5-6 with 1M HCl and extracted with ethyl acetate (30 mL). The combined organic layers were washed with brine (20 mL), dried over sodium sulfate, filtered, and concentrated under reduced pressure to give 2-[4-[3-[3-bromo-2-(trifluoromethyl)phenoxy]propyl]-1-piperidyl]acetic acid (400 mg) as a yellow solid.

LC-MS (ESI): m/z=424.0 [M+H]⁺

### Step 5: 2-[4-[3-[3-bromo-2-(trifluoromethyl)phenoxy]propyl]-1-piperidyl]-N-[3-(2,6-dioxo-3-piperidyl)-1-methyl-indazol-6-yl]acetamide

To a solution of 2-[4-[3-[3-bromo-2-(trifluoromethyl)phenoxy]propyl]-1-piperidyl]acetic acid (300 mg, 707.13 mol, 1.0 equiv.) and 3-(6-amino-1-methyl-indazol-3-yl)piperidine-2,6-dione (219.16 mg, 848.55 mol, 1.2 equiv.) in pyridine (4 mL) was added EDCI (203.34 mg, 1.06 mmol, 1.5 equiv.). The mixture was stirred at 25° C. for 16 hours. The reaction mixture was concentrated under reduced pressure to give a residue, which was purified by flash silica gel chromatography (0~10% MeOH/DCM) to give 2-[4-[3-[3-bromo-2-(trifluoromethyl)phenoxy]propyl]-1-piperidyl]-N-[3-(2,6-dioxo-3-piperidyl)-1-methyl-indazol-6-yl]acetamide (250 mg) as a yellow solid.

LC-MS (ESI): m/z=664.2 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate

A mixture of 2-[4-[3-[3-bromo-2-(trifluoromethyl)phenoxy]propyl]-1-piperidyl]-N-[3-(2,6-dioxo-3-piperidyl)-1-methyl-indazol-6-yl]acetamide (90 mg, 135.44 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (159.06 mg, 162.53 mol, 1.2 equiv.), Ad₂nBuP Pd G₃ (49.32 mg, 67.72 mol, 0.5 equiv.), and KF (23.61 mg, 406.31 mol, 3.0 equiv.) in dioxane (2 mL) and H₂O (0.1 mL) was degassed and purged with N₂ three times. The mixture was stirred at 80° C. for 2 hours under a N₂ atmosphere. The reaction mixture was diluted with water (20 mL) and extracted with DCM (60 mL). The combined organic layers were washed with brine (40 mL), dried over sodium sulfate, filtered, and concentrated under reduced pressure to give a residue, which was purified by flash silica gel chromatography (0~10% MeOH/DCM) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (110 mg) as a yellow solid.

LC-MS (ESI): m/z=1202.6 [M+H]⁺

### Step 7: 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (110 mg, 91.56 mol, 1.0 equiv.) in DCM (1.5 mL) and TFA (1.5 mL) was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid (31.95 mg) as a yellow solid.

LC-MS (ESI): m/z=1015.2 [M+H]⁺

¹H NMR (400 MHz, DMSO-d₆) δ=12.39-12.25 (m, 1H), 10.87 (s, 1H), 10.02-9.67 (m, 1H), 8.57 (d, J=4.0 Hz, 1H), 8.08-8.00 (m, 2H), 7.82 (d, J=8.0 Hz, 1H), 7.65-7.56 (m, 2H), 7.53-7.44 (m, 3H), 7.39-7.33 (m, 1H), 7.21 (d, J=8.0 Hz, 2H), 7.08 (d, J=8.8 Hz, 1H), 6.69 (d, J=7.6 Hz, 1H), 5.33-5.18 (m, 2H), 4.35-4.28 (m, 1H), 4.13-4.02 (m, 2H), 4.04-3.97 (m, 2H), 3.92 (s, 3H), 3.14-3.05 (m, 4H), 2.97-2.85 (m, 2H), 2.67-2.60 (m, 2H), 2.36-2.30 (m, 2H), 2.20-2.13 (m, 2H), 1.79-1.67 (m, 4H), 1.43-1.28 (m, 5H).

### Example 42: 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid (Compound 180)

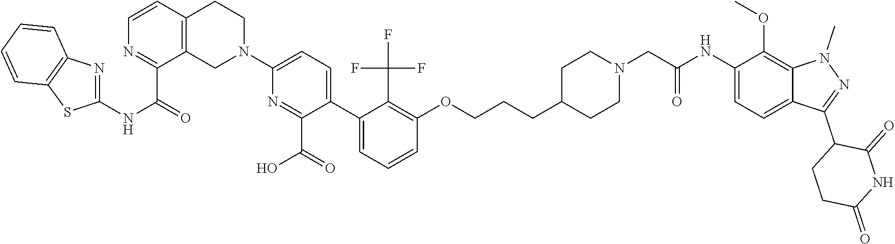 [SMILES: COc1c(NC(=O)CN2CCC(CCCOc3cccc(-c4ccc(N5CCc6ccnc(C(=O)Nc7nc8ccccc8s7)c6C5)nc4C(=O)O)c3C(F)(F)F)CC2)ccc2c(C3CCC(=O)NC3=O)nn(C)c12]

### Step 1: 2-[4-[3-[3-bromo-2-(trifluoromethyl)phenoxy]propyl]-1-piperidyl]-N-[3-(2,6-dioxo-3-piperidyl)-7-methoxy-1-methyl-indazol-6-yl]acetamide

To a solution of 2-[4-[3-[3-bromo-2-(trifluoromethyl)phenoxy]propyl]-1-piperidyl]acetic acid (200 mg, 471.42 mol, 1.0 equiv.) and 3-(6-amino-7-methoxy-1-methyl-indazol-3-yl)piperidine-2,6-dione (149.50 mg, 518.56 mol, 1.1 equiv.) in pyridine (3 mL) was added EDCI (135.56 mg, 707.13 mol, 1.5 equiv.). The mixture was stirred at 40° C. for 2 hours. The reaction mixture was concentrated under reduced pressure to give a residue, which was purified by flash silica gel chromatography (0-7% DCM/MeOH) to give 2-[4-[3-[3-bromo-2-(trifluoromethyl)phenoxy]propyl]-1-piperidyl]-N-[3-(2,6-dioxo-3-piperidyl)-7-methoxy-1-methyl-indazol-6-yl]acetamide (300 mg) as a red solid.

LC-MS (ESI): m/z=694.2 [M+H]⁺

¹H NMR (400 MHz, DMSO-d₆) δ=10.89 (s, 1H), 9.85 (s, 1H), 8.01 (d, J=8.8 Hz, 1H), 7.52-7.43 (m, 2H), 7.39 (d, J=7.6 Hz, 1H), 7.29 (d, J=8.0 Hz, 1H), 4.34 (dd, J=5.2, 9.6 Hz, 1H), 4.16-4.09 (m, 5H), 3.89 (s, 3H), 2.93 (d, J=10.4 Hz, 2H), 2.69-2.58 (m, 2H), 2.30-2.14 (m, 4H), 1.75 (d, J=9.6 Hz, 4H), 1.41 (d, J=1.6 Hz, 2H), 1.34-1.15 (m, 5H).

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate

A mixture of 2-[4-[3-[3-bromo-2-(trifluoromethyl)phenoxy]propyl]-1-piperidyl]-N-[3-(2,6-dioxo-3-piperidyl)-7-methoxy-1-methyl-indazol-6-yl]acetamide (100 mg, 143.98 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (117.80 mg, 158.38 mol, 1.1 equiv.), KF (25.09 mg, 431.94 mol, 3.0 equiv.), and Ad₂nBuP Pd G₃ (52.43 mg, 71.99 mol, 0.5 equiv.) in dioxane (2 mL) and H₂O (0.2 mL) was degassed and purged with N₂ three times. The mixture was stirred at 80° C. for 2 hours under a N₂ atmosphere. The reaction mixture was concentrated under reduced pressure to give a residue, which was purified by prep-TLC (SiO₂, DCM:MeOH=15:1) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate (85 mg) as a yellow solid

LC-MS (ESI): m/z=1232.6 [M+H]⁺

### Step 3: 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid

To a solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylate (85 mg, 69.02 mol, 1.0 equiv.) in DCM (1 mL) and TFA (1 mL) was stirred at 40° C. for 1.5 hours. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[3-[1-[2-[[3-(2,6-dioxo-3-piperidyl)-7-methoxy-1-methyl-indazol-6-yl]amino]-2-oxo-ethyl]-4-piperidyl]propoxy]-2-(trifluoromethyl)phenyl]pyridine-2-carboxylic acid (23.26 mg) as a yellow solid.

LC-MS (ESI): m/z=523.4 [M/2+H]⁺.

¹H NMR (400 MHz, DMSO-d₆) δ=12.62-12.46 (m, 1H), 12.31 (s, 1H), 10.89 (s, 1H), 9.96-9.73 (m, 1H), 8.57 (d, J=4.8 Hz, 1H), 8.11-7.88 (m, 2H), 7.81 (d, J=8.0 Hz, 1H), 7.59 (d, J=5.2 Hz, 1H), 7.52-7.44 (m, 4H), 7.38-7.34 (m, 1H), 7.22 (d, J=8.8 Hz, 1H), 7.09 (d, J=8.8 Hz, 1H), 6.70 (d, J=7.6 Hz, 1H), 5.32-5.20 (m, 2H), 4.34 (dd, J=5.2, 10.0 Hz, 1H), 4.15-4.10 (m, 5H), 4.04-3.96 (m, 2H), 3.88 (s, 3H), 3.20-3.11 (m, 2H), 3.08 (t, J=6.0 Hz, 2H), 3.01-2.87 (m, 2H), 2.68-2.61 (m, 2H), 2.34-2.16 (m, 4H), 1.83-1.74 (m, 4H), 1.45 (dd, J=2.4, 3.2 Hz, 2H), 1.38-1.22 (m, 3H).

### Example 43: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (Compound 160)

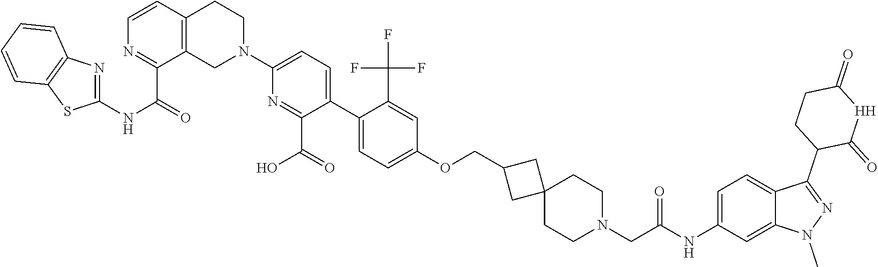

### Step 1: tert-butyl 2-[[4-bromo-3-(trifluoromethyl)phenoxy]methyl]-7-azaspiro[3.5]nonane-7-carboxylate

A mixture of tert-butyl 2-(hydroxymethyl)-7-azaspiro[3.5]nonane-7-carboxylate (1.0 g, 3.92 mmol, 1.0 equiv.), 4-bromo-3-(trifluoromethyl)phenol (1.04 g, 4.31 mmol, 1.1 equiv.), and 2-(tributyl-phosphanylidene)acetonitrile (1.42 g, 5.87 mmol, 1.5 equiv.) in toluene (50 mL) was degassed and purged with N₂ three times. The mixture was stirred at 125° C. for 12 hours under a N₂ atmosphere. The reaction mixture was concentrated under reduced pressure to give a residue, which was purified by flash silica gel chromatography (0~8% ethyl acetate/petroleum ether) to give tert-butyl 2-[[4-bromo-3-(trifluoromethyl)phenoxy]methyl]-7-azaspiro[3.5]nonane-7-carboxylate (1.5 g) as a yellow oil

LC-MS (ESI): m/z=422.0 [M−56+H]⁺.

### Step 2: 2-[[4-bromo-3-(trifluoromethyl)phenoxy]methyl]-7-azaspiro[3.5]nonane

A solution of tert-butyl 2-[[4-bromo-3-(trifluoromethyl)phenoxy]methyl]-7-azaspiro[3.5]nonane-7-carboxylate (1.5 g, 3.14 mmol, 1.0 equiv.) in HCl/dioxane (15 mL) was stirred at 25° C. for 12 hours. The mixture was concentrated under reduced pressure to give 2-[[4-bromo-3-(trifluoromethyl)phenoxy]methyl]-7-azaspiro[3.5]nonane (800 mg, crude) as a white solid.

LC-MS (ESI): m/z=379.0 [M+H]⁺

### Step 3: ethyl 2-[2-[[4-bromo-3-(trifluoromethyl)phenoxy]methyl]-7-azaspiro[3.5]nonan-7-yl]acetate

To a solution of 2-[[4-bromo-3-(trifluoromethyl)phenoxy]methyl]-7-azaspiro[3.5]nonane (400 mg, 1.06 mmol, 1.0 equiv.) and ethyl 2-bromoacetate (176.61 mg, 1.06 mmol, 117.04 μL, 1.0 equiv.) in DMF (5 mL) was added potassium carbonate (438.48 mg, 3.17 mmol, 3.0 equiv.). The mixture was stirred at 40° C. for 1 hour. The reaction mixture was partitioned between ethyl acetate (30 mL) and water (20 mL). The organic phase was separated, washed with brine (60 mL), dried over sodium sulfate, filtered, and concentrated under reduced pressure to give a residue, which was purified by flash silica gel chromatography (0-30% ethyl acetate/petroleum ether) to give ethyl 2-[2-[[4-bromo-3-(trifluoromethyl)phenoxy]methyl]-7-azaspiro[3.5]nonan-7-yl]acetate (350 mg) as a yellow oil.

### Step 4: 2-[2-[[4-bromo-3-(trifluoromethyl)phenoxy]methyl]-7-azaspiro[3.5]nonan-7-yl]acetic acid

To a solution of ethyl 2-[2-[[4-bromo-3-(trifluoromethyl)phenoxy]methyl]-7-azaspiro[3.5]nonan-7-yl]acetate (350 mg, 753.80 mol, 1.0 equiv.) in THE (5 mL) and H₂O (1 mL) was added LiOH·H₂O (94.90 mg, 2.26 mmol, 3.0 equiv.). The mixture was stirred at 25° C. for 12 hours. The reaction mixture was concentrated under reduced pressure to give 2-[2-[[4-bromo-3-(trifluoromethyl)phenoxy]methyl]-7-azaspiro[3.5]nonan-7-yl]acetic acid (350 mg, crude) as a white solid.

LC-MS (ESI): m/z=436.0 [M+H]⁺

### Step 5: 2-[2-[[4-bromo-3-(trifluoromethyl)phenoxy]methyl]-7-azaspiro[3.5]nonan-7-yl]-N-[3-(2,6-dioxo-3-piperidyl)-1-methyl-indazol-6-yl]acetamide

To a solution of 2-[2-[[4-bromo-3-(trifluoromethyl)phenoxy]methyl]-7-azaspiro[3.5]nonan-7-yl]acetic acid (150 mg, 343.83 mol, 1.0 equiv.) and 3-(6-amino-1-methyl-indazol-3-yl)piperidine-2,6-dione (133.20 mg, 515.74 mol, 1.5 equiv.) in pyridine (0.5 mL) was added EDCI (131.82 mg, 687.66 mol, 2.0 equiv.). The mixture was stirred at 25° C. for 1 hour. The reaction mixture was concentrated under reduced pressure to give a residue, which was purified by flash silica gel chromatography (0-75% ethyl acetate/petroleum ether) to give 2-[2-[[4-bromo-3-(trifluoromethyl)phenoxy]methyl]-7-azaspiro[3.5]nonan-7-yl]-N-[3-(2,6-dioxo-3-piperidyl)-1-methyl-indazol-6-yl]acetamide (110 mg) as a yellow solid.

LC-MS (ESI): m/z=676.1 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate

A mixture of 2-[2-[[4-bromo-3-(trifluoromethyl)phenoxy]methyl]-7-azaspiro[3.5]nonan-7-yl]-N-[3-(2,6-dioxo-3-piperidyl)-1-methyl-indazol-6-yl]acetamide (99.22 mg, 146.67 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (120 mg, 161.34 mol, 1.1 equiv.), Ad₂nBuP Pd G₃ (53.41 mg, 73.33 mol, 0.5 equiv.), and KF (25.56 mg, 440.01 mol, 3.0 equiv.) in dioxane (2 mL) and H₂O (0.2 mL) was degassed and purged with N₂ three times. The mixture was stirred at 80° C. for 2 hours under a N₂ atmosphere. The reaction mixture was concentrated under reduced pressure to give a residue, which was purified by flash silica gel chromatography (0~80% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate (130 mg) as a yellow solid.

LC-MS (ESI): m/z=607.4 [M/2+H]⁺.

### Step 7: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate (130 mg, 107.13 mol, 1.0 equiv.) in DCM (1 mL) and TFA (2 mL) was stirred at 40° C. for 0.5 hours. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (38.06 mg) as a white solid.

LC-MS (ESI): m/z=1027.2 [M+H]⁺

¹H NMR (400 MHz, DMSO-d₆) δ=12.51 (m, 1H), 12.31 (s, 1H), 10.88 (s, 1H), 9.83-9.79 (m, 1H), 8.57 (d, J=4.0 Hz, 1H), 8.04-7.99 (m, 2H), 7.82 (d, J=8.0 Hz, 1H), 7.73-7.62 (m, 1H), 7.59 (d, J=4.0 Hz, 1H), 7.52-7.45 (m, 2H), 7.39-7.33 (m, 1H), 7.24-7.14 (m, 4H), 7.09 (d, J=8.8 Hz, 1H), 5.34-5.19 (m, 2H), 4.33 (dd, J=5.6, 8.8 Hz, 1H), 4.08-3.97 (m, 4H), 3.93 (s, 3H), 3.23 (d, J=4.4 Hz, 2H), 3.08 (t, J=5.6 Hz, 2H), 2.95-2.82 (m, 1H), 2.76-2.61 (m, 4H), 2.57 (d, J=2.8, 1H), 2.39-2.34 (m, 1H), 2.20-2.16 (m, 1H), 2.03-1.87 (m, 4H), 1.74-1.60 (m, 5H).

### Example 44: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (Compound 181)

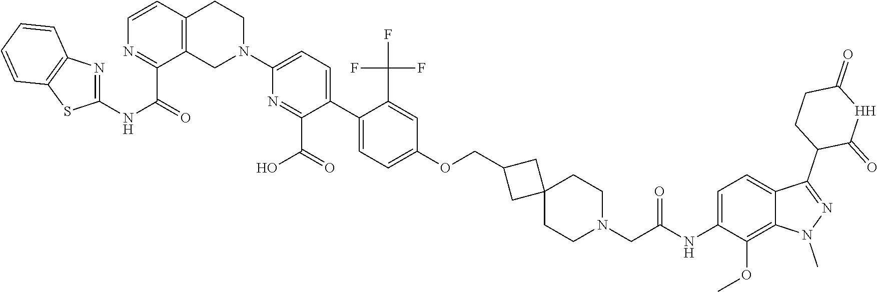

### Step 1: 2-[2-[[4-bromo-3-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide

To a solution of 3-(6-amino-7-methoxy-1-methyl-indazol-3-yl)piperidine-2,6-dione (109.04 mg, 378.21 mol, 1.1 equiv.) and 2-[2-[[4-bromo-3-(trifluoromethyl)phenoxy]methyl]-7-azaspiro[3.5]nonan-7-yl]acetic acid (150 mg, 343.83 mol, 1.0 equiv.) in pyridine (2 mL) was added EDCI (98.87 mg, 515.74 mol, 1.5 equiv.). The mixture was stirred at 40° C. for 1 hour. The reaction mixture was concentrated under reduced pressure to remove pyridine. The residue was diluted with H₂O (50 mL) and extracted with DCM (90 mL). The combined organic layers were washed with brine (60 mL), dried over anhydrous sodium sulfate, filtered, and concentrated under reduced pressure to give 2-[2-[[4-bromo-3-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide (120 mg) as a red solid.

LC-MS (ESI): m/z=708.1 [M+H]⁺

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate

A mixture of 2-[2-[[4-bromo-3-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide (105 mg, 148.61 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (121.59 mg, 163.47 mol, 1.1 equiv.), KF (25.90 mg, 445.83 mol, 3.0 equiv.), and Ad₂nBuP Pd G₃ (54.11 mg, 74.30 mol, 0.5 equiv.) in dioxane (2.5 mL) and H₂O (0.25 mL) was degassed and purged with N₂ three times. The mixture was stirred at 80° C. for 1 hour under a N₂ atmosphere. The reaction mixture was filtered and concentrated under reduced pressure to give a residue, which was purified by flash silica gel chromatography (0~5% methanol/dichloromethane) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate (120 mg) as a yellow solid

LC-MS (ESI): m/z=622.5 [M/2+H].

### Step 3: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate (120 mg, 57.90 mol, 1.0 equiv.) in TFA (2 mL) and DCM (1 mL) was stirred at 40° C. for 1 hour. The reaction mixture was filtered and concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (19.72 mg) as an off-white solid.

LC-MS (ESI): m/z=1057.2 [M+H]⁺

¹H NMR (400 MHz, DMSO-d₆) δ=12.90-12.04 (m, 2H), 10.90 (s, 1H), 9.85 (s, 1H), 8.57 (d, J=3.6 Hz, 1H), 8.03 (dd, J=7.6, 17.6 Hz, 2H), 7.81 (d, J=8.0 Hz, 1H), 7.59 (d, J=3.6 Hz, 1H), 7.54-7.42 (m, 3H), 7.6 (t, J=8.0 Hz, 1H), 7.25-7.14 (m, 3H), 7.08 (d, J=8.8 Hz, 1H), 5.27 (dd, J=18.4, 30.4 Hz, 2H), 4.34 (dd, J=5.2, 10.0 Hz, 1H), 4.14 (s, 3H), 4.06 (d, J=6.4 Hz, 2H), 4.04-3.97 (m, 2H), 3.89 (s, 3H), 3.14 (s, 2H), 3.08 (t, J=5.6 Hz, 2H), 2.76-2.69 (m, 1H), 2.68-2.61 (m, 2H), 2.60-2.53 (m, 2H), 2.48-2.43 (m, 2H), 2.39-2.29 (m, 1H), 2.22-2.11 (m, 1H), 1.98 (t, J=10.4 Hz, 2H), 1.74 (s, 2H), 1.71-1.62 (m, 4H).

### Example 45: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (Compound 161)

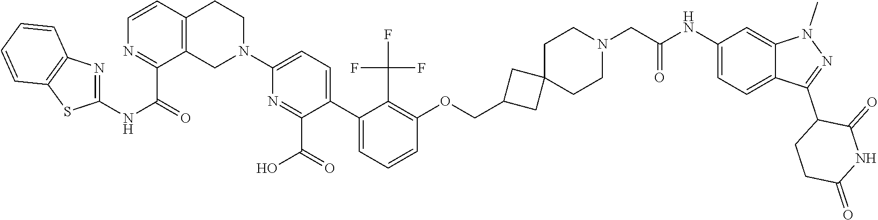 [SMILES: Cn1nc(C2CCC(=O)NC2=O)c2ccc(NC(=O)CN3CCC4(CC3)CC(COc3cccc(-c5ccc(N6CCc7ccnc(C(=O)Nc8nc9ccccc9s8)c7C6)nc5C(=O)O)c3C(F)(F)F)C4)cc21]

### Step 1: tert-butyl 2-((3-bromo-2-(trifluoromethyl)phenoxy)methyl)-7-azaspiro[3.5]nonane-7-carboxylate

A mixture of tert-butyl 2-(hydroxymethyl)-7-azaspiro[3.5]nonane-7-carboxylate (1.0 g, 3.92 mmol, 1.0 equiv.), 3-bromo-2-(trifluoromethyl)phenol (1.04 g, 4.31 mmol, 1.1 equiv.) and 2-(tributyl-phosphanylidene)acetonitrile (1.42 g, 5.87 mmol, 1.5 equiv.) in toluene (10 mL) was degassed and purged with N₂ three times. The mixture was stirred at 130° C. for 2 hours in a microwave reactor. The reaction mixture was filtered and concentrated under reduced pressure to give a residue, which was purified by prep-HPLC to give tert-butyl 2-((3-bromo-2-(trifluoromethyl)phenoxy)methyl)-7-azaspiro[3.5]nonane-7-carboxylate (1.5 g) as a brown oil.

¹H NMR (400 MHz, DMSO-d₆) δ=7.47 (t, J=8.0 Hz, 1H), 7.38 (d, J=8.0 Hz, 1H), 7.28 (d, J=8.4 Hz, 1H), 4.07 (d, J=6.0 Hz, 2H), 3.29-3.26 (m, 2H), 3.19-3.16 (m, 2H), 2.76-2.64 (m, 1H), 1.87 (t, J=9.2 Hz, 2H), 1.67 (t, J=8.8 Hz, 2H), 1.53 (t, J=5.2 Hz, 2H), 1.42-1.38 (m, 12H)

### Step 2: 2-[[3-bromo-2-(trifluoromethyl)phenoxy]methyl]-7-azaspiro[3.5]nonane

A solution of tert-butyl 2-((3-bromo-2-(trifluoromethyl)phenoxy)methyl)-7-azaspiro[3.5]nonane-7-carboxylate (1.5 g, 3.14 mmol, 1.0 equiv.) in HCl/dioxane (2 M, 20 mL) was stirred at 25° C. for 2 hours. The reaction mixture was filtered under reduced pressure to give 2-[[3-bromo-2-(trifluoromethyl)phenoxy]methyl]-7-azaspiro[3.5]nonane (1.0 g) as a white solid.

LC-MS (ESI): m/z=379.9 [M+H]⁺

### Step 3: ethyl 2-[2-[[3-bromo-2-(trifluoromethyl)phenoxy]methyl]-7-azaspiro[3.5]nonan-7-yl]acetate

To a solution of 2-[[3-bromo-2-(trifluoromethyl)phenoxy]methyl]-7-azaspiro[3.5]nonane (1 g, 2.64 mmol, 1.0 equiv.) and ethyl 2-bromoacetate (441.54 mg, 2.64 mmol, 292.60 μL, 1.0 equiv.) in DMF (20 mL) was added potassium carbonate (1.10 g, 7.93 mmol, 3.0 equiv.). The mixture was stirred at 40° C. for 1 hour. The reaction mixture was diluted with H₂O (100 mL) and extracted with EtOAc (150 mL). The combined organic layers were washed with H₂O (150 mL), dried over sodium sulfate, filtered, and concentrated under reduced pressure to give ethyl 2-[2-[[3-bromo-2-(trifluoromethyl)phenoxy]methyl]-7-azaspiro[3.5]nonan-7-yl]acetate (1.1 g) as a brown oil.

LC-MS (ESI): m/z=465.8 [M+H]⁺

### Step 4: 2-[2-[[3-bromo-2-(trifluoromethyl)phenoxy]methyl]-7-azaspiro[3.5]nonan-7-yl]acetic acid

To a solution of ethyl 2-[2-[[3-bromo-2-(trifluoromethyl)phenoxy]methyl]-7-azaspiro[3.5]nonan-7-yl]acetate (1.1 g, 2.37 mmol, 1.0 equiv.) in THF (10 mL) and H₂O (2 mL) was added LiOH·H₂O (298.25 mg, 7.11 mmol, 3.0 equiv.). The mixture was stirred at 40° C. for 2 hours. The pH of the mixture was adjusted to pH=5-6 with 1M HCl and extracted with ethyl acetate (30 mL). The combined organic layers were washed with brine (20 mL), dried over sodium sulfate, filtered, and concentrated under reduced pressure to give 2-[2-[[3-bromo-2-(trifluoromethyl)phenoxy]methyl]-7-azaspiro[3.5]nonan-7-yl]acetic acid (950 mg) as a white solid.

¹H NMR (400 MHz, DMSO-d₆) δ=7.46 (t, J=8.0 Hz, 1H), 7.38 (d, J=8.0 Hz, 1H), 7.28 (d, J=8.4 Hz, 1H), 4.04 (d, J=6.0 Hz, 2H), 2.68-2.62 (m, 1H), 2.59 (s, 2H), 2.45-2.03 (m, 4H), 1.81 (t, J=10.8 Hz, 2H), 1.69-1.53 (m, 4H), 1.48 (t, J=4.8 Hz, 2H).

### Step 5: 2-[2-[[3-bromo-2-(trifluoromethyl)phenoxy]methyl]-7-azaspiro[3.5]nonan-7-yl]-N-[3-(2,6-dioxo-3-piperidyl)-1-methyl-indazol-6-yl]acetamide

To a solution of 2-[2-[[3-bromo-2-(trifluoromethyl)phenoxy]methyl]-7-azaspiro[3.5]nonan-7-yl]acetic acid (150 mg, 343.83 mol, 1.0 equiv.) and 3-(6-amino-1-methyl-indazol-3-yl)piperidine-2,6-dione (106.56 mg, 412.60 mol, 1.2 equiv.) in pyridine (2 mL) was added EDCI (85.69 mg, 446.98 mol, 1.3 equiv.). The mixture was stirred at 25° C. for 2 hours. The reaction mixture was filtered and concentrated under reduced pressure to give a residue, which was partitioned between DCM (90 mL) and H₂O (60 mL). The separated organic layer was washed with brine (30 mL), dried over sodium sulfate, filtered, and concentrated in vacuum. The residue was purified by flash silica gel chromatography (0-19% methanol:dichloromethane) to give 2-[2-[[3-bromo-2-(trifluoromethyl)phenoxy]methyl]-7-azaspiro[3.5]nonan-7-yl]-N-[3-(2,6-dioxo-3-piperidyl)-1-methyl-indazol-6-yl]acetamide (190 mg) as a blue solid.

LC-MS (ESI): m/z=678.2 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate

A mixture of 2-[2-[[3-bromo-2-(trifluoromethyl)phenoxy]methyl]-7-azaspiro[3.5]nonan-7-yl]-N-[3-(2,6-dioxo-3-piperidyl)-1-methyl-indazol-6-yl]acetamide (100 mg, 147.81 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (120.94 mg, 162.60 mol, 1.1 equiv.), Ad₂nBuP Pd G₃ (53.82 mg, 73.91 mol, 0.5 equiv.), and KF (25.76 mg, 443.44 mol, 3.0 equiv.) in dioxane (2 mL) and H₂O (0.2 mL) was degassed and purged with N₂ three times. The mixture was stirred at 80° C. for 2 hours under a N₂ atmosphere. The reaction mixture was filtered and concentrated under reduced pressure to give a residue, which was purified by flash silica gel chromatography (0~10% MeOH:DCM) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate (90 mg) as a yellow solid.

LC-MS (ESI): m/z=607.5 [M/2+H]⁺

### Step 7: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylate (90 mg, 74.17 mol, 1.0 equiv.) in TFA (2 mL) and DCM (1 mL) was stirred at 40° C. for 1 hour. The reaction mixture was filtered and concentrated under reduced pressure to give a residue, which was purified by reversed-phase HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (33.45 mg) as an off-white solid.

LC-MS (ESI): m/z=1027.2 [M+H]⁺

¹H NMR (400 MHz, DMSO-d₆) δ=12.31 (s, 1H), 10.87 (s, 1H), 9.86 (s, 1H), 8.57 (d, J=4.8 Hz, 1H), 8.10-7.98 (m, 2H), 7.81 (d, J=8.0 Hz, 1H), 7.63 (d, J=8.4 Hz, 1H), 7.58 (d, J=4.8 Hz, 1H), 7.54-7.42 (m, 3H), 7.36 (t, J=7.6 Hz, 1H), 7.21 (dd, J=4.8, 8.0 Hz, 2H), 7.08 (d, J=8.8 Hz, 1H), 6.69 (d, J=7.6 Hz, 1H), 5.34-5.17 (m, 2H), 4.32 (dd, J=5.2, 10.0 Hz, 1H), 4.11-4.03 (m, 2H), 4.03-3.95 (m, 2H), 3.92 (s, 3H), 3.56-3.47 (m, 1H), 3.11-3.19 (m, 2H), 3.08 (t, J=5.6 Hz, 2H), 2.75-2.69 (m, 1H), 2.68-2.60 (m, 2H), 2.47-2.40 (m, 2H), 2.40-2.24 (m, 2H), 2.21-2.14 (m, 1H), 1.88 (t, J=10.0 Hz, 2H), 1.77-1.68 (m, 4H), 1.64-1.56 (m, 2H).

### Example 46: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (Compound 162)

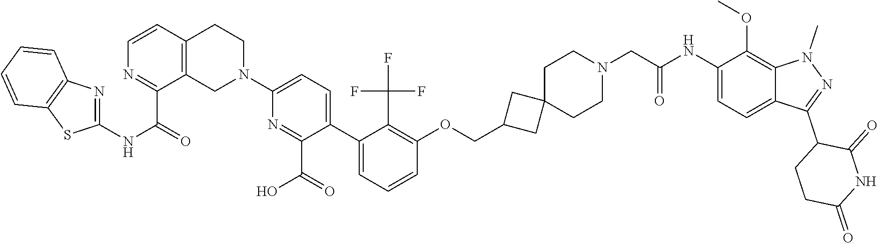 [SMILES: COc1c(NC(=O)CN2CCC3(CC2)CC(COc2cccc(-c4ccc(N5CCc6ccnc(C(=O)Nc7nc8ccccc8s7)c6C5)nc4C(=O)O)c2C(F)(F)F)C3)ccc2c(C3CCC(=O)NC3=O)nn(C)c12]

### Step 1: 2-[2-[[3-bromo-2-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide

To a solution of 2-[2-[[3-bromo-2-(trifluoromethyl)phenoxy]methyl]-7-azaspiro[3.5]nonan-7-yl]acetic acid (200 mg, 458.44 mol, 1.0 equiv.) and 3-(6-amino-7-methoxy-1-methyl-indazol-3-yl)piperidine-2,6-dione (158.60 mg, 550.13 mol, 1.2 equiv.) in pyridine (2 mL) was added EDCI (114.25 mg, 595.97 mol, 1.3 equiv.). The mixture was stirred at 25° C. for 1 hour. The reaction mixture was concentrated under reduced pressure, diluted with H₂O (30 mL), and extracted with EtOAc (90 mL). The combined organic layers were washed with H₂O (100 mL), dried over sodium sulfate, filtered, and concentrated under reduced pressure to give a residue, which was purified by flash silica gel chromatography (0-100 ethyl acetate/petroleum ether) to give 2-[2-[[3-bromo-2-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide (200 mg) as a light yellow solid.

LC-MS (ESI): m/z=708.0 [M+H]⁺

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate

A mixture of 2-[2-[[3-bromo-2-(trifluoromethyl)phenoxy]methyl]-7-azaspiro[3.5]nonan-7-yl]-N-[3-(2,6-dioxo-3-piperidyl)-7-methoxy-1-methyl-indazol-6-yl]acetamide (100 mg, 113.23 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (101.06 mg, 135.87 mol, 1.2 equiv.), Ad₂nBuP Pd G₃ (41.23 mg, 56.61 mol, 0.5 equiv.), and KF (19.73 mg, 339.68 mol, 3.0 equiv.) in dioxane (1 mL) and H₂O (0.1 mL) was degassed and purged with N₂ three times. The mixture was stirred at 80° C. for 2 hours under a N₂ atmosphere. The reaction mixture was concentrated under reduced pressured to give a residue, which was purified by flash silica gel chromatography (0~85% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate (100 mg) as a yellow solid.

LC-MS (ESI): m/z=1265.4 [M+Na]*.

### Step 3: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylate (100 mg, 80.42 mol, 1.0 equiv.) in TFA (2 mL) and DCM (1 mL) was stirred at 40° C. for 1 hour. The reaction mixture was concentrated under reduced pressure to give a residue, which was by purified by prep-HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]methoxy]-2-(trifluoromethyl)phenyl]pyridine-2-carboxylic acid (25.06 mg) as an off-white solid.

LC-MS (ESI): m/z=1057.2 [M+H]⁺

¹H NMR (400 MHz, DMSO-d₆) δ=12.65-12.25 (m, 1H), 10.89 (s, 1H), 9.85 (s, 1H), 8.57 (d, J=4.8 Hz, 1H), 8.03 (dd, J=8.4, 18.8 Hz, 2H), 7.81 (d, J=8.0 Hz, 1H), 7.58 (d, J=4.8 Hz, 1H), 7.53-7.44 (m, 4H), 7.40-7.32 (m, 1H), 7.21 (d, J=8.8 Hz, 1H), 7.07 (d, J=8.8 Hz, 1H), 6.70 (d, J=7.6 Hz, 1H), 5.33-5.19 (m, 2H), 4.33 (dd, J=5.2, 9.6 Hz, 1H), 4.13 (s, 3H), 4.10-4.04 (m, 2H), 4.03-3.93 (m, 2H), 3.89 (s, 3H), 3.13 (s, 2H), 3.08 (t, J=5.6 Hz, 2H), 2.76-2.69 (m, 1H), 2.68-2.60 (m, 2H), 2.60-2.53 (m, 2H), 2.46 (s, 2H), 2.39-2.29 (m, 1H), 2.20-2.12 (m, 1H), 1.94-1.87 (m, 2H), 1.78-1.69 (m, 4H), 1.63 (t, J=4.4 Hz, 2H).

### Example 47: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (Compound 163)

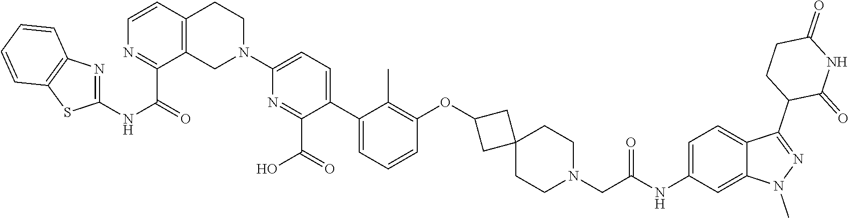 [SMILES: Cc1c(OC2CC3(CCN(CC(=O)Nc4ccc5c(C6CCC(=O)NC6=O)nn(C)c5c4)CC3)C2)cccc1-c1ccc(N2CCc3ccnc(C(=O)Nc4nc5ccccc5s4)c3C2)nc1C(=O)O]

### Step 1: tert-butyl 2-(3-bromo-2-methyl-phenoxy)-7-azaspiro[3.5]nonane-7-carboxylate

3-Bromo-2-methyl-phenol (852.53 mg, 4.56 mmol, 1.1 equiv.), tert-butyl 2-hydroxy-7-azaspiro[3.5]nonane-7-carboxylate (1 g, 4.14 mmol, 1.0 equiv.), and 2-(tributyl-λ⁵-phosphanylidene)acetonitrile (1.20 g, 4.97 mmol, 1.2 equiv.) were taken up into a microwave tube in toluene (10 mL). The sealed tube was heated at 130° C. for 1 hour in a microwave reactor. The reaction mixture was filtered and concentrated under reduced pressure to give a residue, which was purified by flash silica gel chromatography (0~3% ethyl acetate/petroleum ether) to give tert-butyl 2-(3-bromo-2-methyl-phenoxy)-7-azaspiro[3.5]nonane-7-carboxylate (1.5 g) as a colorless oil.

¹H NMR (400 MHz, DMSO-d₆) δ=7.16-7.10 (m, 1H), 7.09-7.03 (m, 1H), 6.77 (d, J=8.4 Hz, 1H), 4.80-4.69 (m, 1H), 3.30-3.25 (m, 2H), 3.24-3.18 (m, 2H), 2.45-2.35 (m, 2H), 2.22 (s, 3H), 1.85-1.74 (m, 2H), 1.57-1.44 (m, 4H), 1.38 (s, 9H)

### Step 2: 2-(3-bromo-2-methyl-phenoxy)-7-azaspiro[3.5]nonane

To a solution of tert-butyl 2-(3-bromo-2-methyl-phenoxy)-7-azaspiro[3.5]nonane-7-carboxylate (1.5 g, 2.95 mmol, 1.0 equiv.) in DCM (7 mL) was added HCl/dioxane (2 M, 7 mL, 4.75 equiv.) at 25° C. The reaction was stirred at 25° C. for 2 hours. The reaction mixture was concentrated under reduced pressure to give a residue, which was triturated with DCM at 25° C. for 10 mins and filtered to give 2-(3-bromo-2-methyl-phenoxy)-7-azaspiro[3.5]nonane (1 g) as a yellow solid.

¹H NMR (400 MHz, DMSO-d₆) δ=7.19-7.12 (m, 1H), 7.10-7.03 (m, 1H), 6.78 (d, J=8.0 Hz, 1H), 4.83-4.66 (m, 1H), 3.06-2.86 (m, 4H), 2.48-2.43 (m, 2H), 2.23 (s, 3H), 1.91-1.68 (m, 6H).

### Step 3: ethyl 2-[2-(3-bromo-2-methyl-phenoxy)-7-azaspiro[3.5]nonan-7-yl]acetate

To a solution of 2-(3-bromo-2-methyl-phenoxy)-7-azaspiro[3.5]nonane (400 mg, 1.29 mmol, 1.0 equiv.) in DMF (4 mL) was added ethyl 2-bromoacetate (226.09 mg, 1.35 mmol, 149.83 μL, 1.05 equiv.) and TEA (652.35 mg, 6.45 mmol, 897.32 μL, 5.0 equiv.) at 25° C. The reaction was stirred at 60° C. for 1 hour. The reaction mixture was diluted with water (4 mL) and extracted with ethyl acetate (30 mL). The combined organic layers were washed with brine (60 mL), dried over sodium sulfate, filtered, and concentrated under reduced pressure to give a residue, which was purified by flash silica gel chromatography (0~50% ethyl acetate/petroleum ether) to give ethyl 2-[2-(3-bromo-2-methyl-phenoxy)-7-azaspiro[3.5]nonan-7-yl]acetate (400 mg) as a yellow oil.

LC-MS (ESI): m/z=396.0 [M+H]⁺

¹H NMR (400 MHz, DMSO-d₆) δ=7.17-7.11 (m, 1H), 7.09-7.02 (m, 1H), 6.77 (d, J=8.0 Hz, 1H), 4.77-4.68 (m, 1H), 4.06 (q, J=7.2 Hz, 2H), 3.14 (s, 2H), 2.47-2.41 (m, 2H), 2.40-2.33 (m, 4H), 2.22 (s, 3H), 1.81-1.69 (m, 2H), 1.62-1.48 (m, 4H), 1.18 (t, J=7.2 Hz, 3H)

### Step 4: 2-[2-(3-bromo-2-methyl-phenoxy)-7-azaspiro[3.5]nonan-7-yl]acetic acid

To a solution of ethyl 2-[2-(3-bromo-2-methyl-phenoxy)-7-azaspiro[3.5]nonan-7-yl]acetate (150 mg, 378.48 mol, 1.0 equiv.) in THE (1.5 mL) and H₂O (0.1 mL) was added LiOH·H₂O (47.65 mg, 1.14 mmol, 3.0 equiv.) at 25° C. The reaction was stirred at 40° C. for 1 hour. The reaction mixture was filtered to give 2-[2-(3-bromo-2-methyl-phenoxy)-7-azaspiro[3.5]nonan-7-yl]acetic acid (148 mg, crude) as a white solid.

LC-MS (ESI): m/z=368.0 [M+H]⁺

### Step 5: 2-[2-(3-bromo-2-methyl-phenoxy)-7-azaspiro[3.5]nonan-7-yl]-N-[3-(2,6-dioxo-3-piperidyl)-1-methyl-indazol-6-yl]acetamide

To a solution of 2-[2-(3-bromo-2-methyl-phenoxy)-7-azaspiro[3.5]nonan-7-yl]acetic acid (148 mg, 401.88 mol, 1.0 equiv.) in pyridine (1.5 mL) was added 3-(6-amino-1-methyl-indazol-3-yl) piperidine-2,6-dione (124.56 mg, 482.26 mol, 1.2 equiv.) and EDCI (100.15 mg, 522.45 mol, 1.3 equiv.) at 25° C. The reaction was stirred at 25° C. for 16 hours. The reaction mixture was concentrated under reduced pressure to give a residue, which was purified by flash silica gel chromatography (0~100% ethyl acetate/petroleum ether) to give 2-[2-(3-bromo-2-methyl-phenoxy)-7-azaspiro[3.5]nonan-7-yl]-N-[3-(2,6-dioxo-3-piperidyl)-1-methyl-indazol-6-yl]acetamide (120 mg) as a yellow solid.

LC-MS (ESI): m/z=608.1 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate

A mixture of 2-[2-(3-bromo-2-methyl-phenoxy)-7-azaspiro[3.5]nonan-7-yl]-N-[3-(2,6-dioxo-3-piperidyl)-1-methyl-indazol-6-yl]acetamide (100 mg, 164.33 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (190 mg, 196.69 mol, 1.20 equiv.), KF (28.64 mg, 493.00 mol, 3.0 equiv.), and Ad₂nBup Pd G₃ (59.84 mg, 82.17 mol, 0.5 equiv.) in dioxane (1 mL) and H₂O (0.1 mL) was degassed and purged with N₂ three times. The mixture was stirred at 80° C. for 3 hours under a N₂ atmosphere. The reaction mixture was concentrated under reduced pressure to give a residue, which was purified by flash silica gel chromatography (0~50% ethyl acetate/DCM) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (150 mg) as a yellow solid.

### Step 7: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (150 mg, 130.95 mol, 1.0 equiv.) in DCM (1 mL) and TFA (1 mL) was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (18.29 mg) as a yellow solid.

LC-MS (ESI): m/z=959.2 [M+H]⁺

¹H NMR (400 MHz, DMSO-d₆) δ=10.87 (s, 1H), 9.84 (s, 1H), 8.57 (d, J=4.8 Hz, 1H), 8.08-8.02 (m, 2H), 7.81 (d, J=8.0 Hz, 1H), 7.63 (d, J=8.8 Hz, 1H), 7.58 (d, J=4.8 Hz, 1H), 7.53-7.45 (m, 2H), 7.40-7.33 (m, 1H), 7.23 (dd, J=1.2, 8.8 Hz, 1H), 7.11-7.03 (m, 2H), 6.72 (d, J=8.0 Hz, 1H), 6.65 (d, J=7.6 Hz, 1H), 5.25 (s, 2H), 4.78-4.67 (m, 1H), 4.32 (dd, J=5.2, 9.6 Hz, 1H), 3.97 (t, J=5.2 Hz, 2H), 3.92 (s, 3H), 3.13 (s, 2H), 3.08 (t, J=5.6 Hz, 2H), 2.72-2.54 (m, 2H), 2.47-2.28 (m, 6H), 2.22-2.12 (m, 1H), 1.91 (s, 3H), 1.87-1.77 (m, 2H), 1.74-1.64 (m, 4H).

### Example 48: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid (Compound 168)

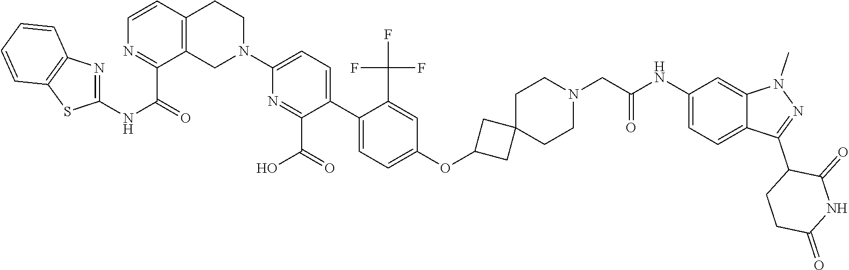 [SMILES: Cn1nc(C2CCC(=O)NC2=O)c2ccc(NC(=O)CN3CCC4(CC3)CC(Oc3ccc(-c5ccc(N6CCc7ccnc(C(=O)Nc8nc9ccccc9s8)c7C6)nc5C(=O)O)c(C(F)(F)F)c3)C4)cc21]

### Step 1: tert-butyl 2-[4-bromo-3-(trifluoromethyl)phenoxy]-7-azaspiro[3.5]nonane-7-carboxylate

A mixture of 4-bromo-3-(trifluoromethyl)phenol (1.20 g, 4.97 mmol, 1.2 equiv.), tert-butyl 2-hydroxy-7-azaspiro[3.5]nonane-7-carboxylate (1.0 g, 4.14 mmol, 1.0 equiv.), and 2-(tributyl-phosphanylidene)acetonitrile (1.50 g, 6.22 mmol, 1.5 equiv.) in toluene (15 mL) was degassed and purged with N₂ three times. The mixture was stirred at 120° C. for 2 hours in a microwave reactor. The reaction mixture was concentrated under reduced pressure to give a residue, which was purified by flash silica gel chromatography (0-2% ethyl acetate/petroleum ether) to give tert-butyl 2-[4-bromo-3-(trifluoromethyl)phenoxy]-7-azaspiro[3.5]nonane-7-carboxylate (1.5 g) as a yellow solid.

¹H NMR (400 MHz, DMSO-d₆) δ=7.74 (d, J=8.8 Hz, 1H), 7.22 (d, J=2.8 Hz, 1H), 7.08 (dd, J=8.8, 2.8 Hz, 1H), 4.91-4.79 (m, 1H), 3.30-3.26 (m, 2H), 3.23-3.18 (m, 2H), 2.45-2.38 (m, 2H), 1.84-1.77 (m, 2H), 1.55-1.51 (m, 2H), 1.49-1.45 (m, 2H), 1.38 (s, 9H).

### Step 2: 2-[4-bromo-3-(trifluoromethyl)phenoxy]-7-azaspiro[3.5]nonane

To a solution of tert-butyl 2-[4-bromo-3-(trifluoromethyl)phenoxy]-7-azaspiro[3.5]nonane-7-carboxylate (1.2 g, 2.58 mmol, 1.0 equiv.) in DCM (2 mL) was added HCl/dioxane (2 M, 20 mL). The mixture was stirred at 25° C. for 1 hour. The reaction mixture was concentrated under reduced pressure to give 2-[4-bromo-3-(trifluoromethyl)phenoxy]-7-azaspiro[3.5]nonane (1.0 g) as a white solid.

LC-MS (ESI): m/z=366.0 [M+H]⁺

### Step 3: ethyl 2-[2-[4-bromo-3-(trifluoromethyl)phenoxy]-7-azaspiro[3.5]nonan-7-yl]acetate

To a solution of 2-[4-bromo-3-(trifluoromethyl)phenoxy]-7-azaspiro[3.5]nonane (500 mg, 1.25 mmol, 1.0 equiv., HCl) in DMF (10 mL) was added potassium carbonate (517.42 mg, 3.74 mmol, 3.0 equiv.) and ethyl 2-bromoacetate (208.41 mg, 1.25 mmol, 138.11 μL, 1.0 equiv.). The mixture was stirred at 40° C. for 1 hour. The reaction mixture was diluted with water (100 mL) and extracted by ethyl acetate (200 mL). The combined organic layers were washed with brine (100 mL), dried over anhydrous sodium sulfate, filtered, and concentrated under reduced pressure to give ethyl 2-[2-[4-bromo-3-(trifluoromethyl)phenoxy]-7-azaspiro[3.5]nonan-7-yl]acetate (500 mg) as a yellow oil.

LC-MS (ESI): m/z=450.1 [M+H]⁺

### Step 4: 2-[2-[4-bromo-3-(trifluoromethyl)phenoxy]-7-azaspiro[3.5]nonan-7-yl]acetic acid

To a solution of ethyl 2-[2-[4-bromo-3-(trifluoromethyl)phenoxy]-7-azaspiro[3.5]nonan-7-yl]acetate (500 mg, 1.11 mmol, 1.0 equiv.) in THE (4 mL) was added LiOH·H₂O (139.79 mg, 3.33 mmol, 3.0 equiv.) and H₂O (1 mL). The mixture was stirred at 40° C. for 1 hour. The reaction mixture was diluted with water (100 mL) and the pH was adjusted to pH=5-6 by HCl (2 M, 2 mL). The mixture was then extracted with DCM/MeOH (10/1) (200 mL). The combined organic layers were dried over anhydrous sodium sulfate, filtered, and concentrated under pressure to give 2-[2-[4-bromo-3-(trifluoromethyl)phenoxy]-7-azaspiro[3.5]nonan-7-yl]acetic acid (460 mg) as a yellow solid.

LC-MS (ESI): m/z=422.0 [M+H]⁺

### Step 5: 2-[2-[4-bromo-3-(trifluoromethyl)phenoxy]-7-azaspiro[3.5]nonan-7-yl]-N-[3-(2,6-dioxo-3-piperidyl)-1-methyl-indazol-6-yl]acetamide

To a solution of 2-[2-[4-bromo-3-(trifluoromethyl)phenoxy]-7-azaspiro[3.5]nonan-7-yl]acetic acid (150 mg, 355.25 mol, 1.0 equiv.) and 3-(6-amino-1-methyl-indazol-3-yl)piperidine-2,6-dione (125.65 mg, 426.30 mol, 1.2 equiv., HCl) in pyridine (4 mL) was added EDCI (102.15 mg, 532.88 mol, 1.5 equiv.). The mixture was stirred at 40° C. for 1 hour. The reaction mixture was concentrated under reduced pressure to give a residue, which was diluted with water (50 mL) and extracted by ethyl acetate (100 mL). The combined organic layers were washed with brine (50 mL), dried over anhydrous sodium sulfate, filtered, and concentrated under vacuum to give a residue, which was purified by flash silica gel chromatography (0-100% ethyl acetate/petroleum ether) to give 2-[2-[4-bromo-3-(trifluoromethyl)phenoxy]-7-azaspiro[3.5]nonan-7-yl]-N-[3-(2,6-dioxo-3-piperidyl)-1-methyl-indazol-6-yl]acetamide (170 mg) as a yellow solid.

LC-MS (ESI): m/z=664.0 [M+H]⁺

### Step 6: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate

A mixture of 2-[2-[4-bromo-3-(trifluoromethyl)phenoxy]-7-azaspiro[3.5]nonan-7-yl]-N-[3-(2,6-dioxo-3-piperidyl)-1-methyl-indazol-6-yl]acetamide (90 mg, 135.85 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (119.51 mg, 149.43 mol, 1.1 equiv.), KF (23.68 mg, 407.55 mol, 3.0 equiv.), H₂O (0.5 mL), and Ad₂nBuP Pd G₃ (49.47 mg, 67.92 mol, 0.5 equiv.) in dioxane (5 mL) was degassed and purged with N₂ three times. The mixture was stirred at 80° C. for 2 hours under a N₂ atmosphere. The reaction mixture was concentrated under reduced pressure to give a residue, which was purified by flash silica gel chromatography (0~10% MeOH/DCM) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate (90 mg) as a yellow solid.

LC-MS (ESI): m/z=600.5 [M/2+H]⁺.

### Step 7: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid

To a solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylate (90 mg, 52.53 mol, 1.0 equiv.) in DCM (1 mL) was added TFA (2 mL). The mixture was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-(trifluoromethyl)phenyl]pyridine-2-carboxylic acid (22.55 mg) as a yellow solid.

LC-MS (ESI): m/z=1013.2 [M+H]⁺

¹H NMR (400 MHz, DMSO-d₆) δ=12.48-12.20 (m, 1H), 10.89 (s, 1H), 10.05-9.85 (m, 1H), 8.63-8.55 (m, 1H), 8.14 (s, 1H), 8.11-8.02 (m, 2H), 7.82 (d, J=7.6 Hz, 1H), 7.64 (d, J=8.8 Hz, 1H), 7.59 (d, J=4.4 Hz, 1H), 7.54-7.46 (m, 2H), 7.40-7.34 (m, 1H), 7.25-7.17 (m, 2H), 7.15-7.07 (m, 3H), 5.27 (d, J=13.6 Hz, 2H), 4.90-4.85 (m, 1H), 4.36-4.29 (m, 2H), 4.01 (d, J=5.6 Hz, 2H), 3.93 (s, 3H), 3.09 (s, 2H), 2.70-2.57 (m, 6H), 2.46-2.43 (m, 2H), 2.40-2.30 (m, 2H), 2.22-2.15 (m, 1H), 1.89-1.82 (m, 2H), 1.72 (d, J=13.2 Hz, 4H).

### Example 49: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid (Compound 169)

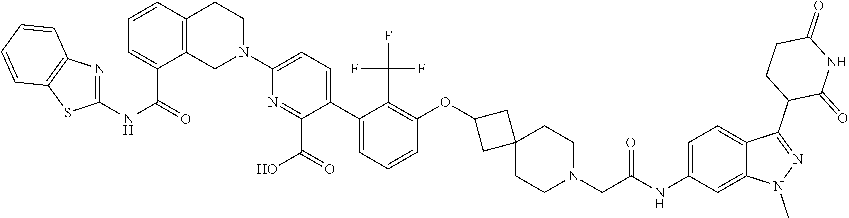 [SMILES: Cn1nc(C2CCC(=O)NC2=O)c2ccc(NC(=O)CN3CCC4(CC3)CC(Oc3cccc(-c5ccc(N6CCc7cccc(C(=O)Nc8nc9ccccc9s8)c7C6)nc5C(=O)O)c3C(F)(F)F)C4)cc21]

### Step 1: tert-butyl 2-(3-bromo-2-(trifluoromethyl)phenoxy)-7-azaspiro[3.5]nonane-7-carboxylate

A mixture of tert-butyl 2-hydroxy-7-azaspiro[3.5]nonane-7-carboxylate (1.0 g, 4.14 mmol, 1 equiv.), 3-bromo-2-(trifluoromethyl)phenol, 2-(tributyl-phosphanylidene)acetonitrile (1.50 g, 6.22 mmol, 1.5 equiv.) in toluene (15 mL) was degassed and purged with N₂ three times. The mixture was stirred at 130° C. for 2 hours under a N₂ atmosphere in a microwave reactor. The reaction mixture was filtered and concentrated under reduced pressure to give a residue, which was purified by reversed-phase HPLC to afford tert-butyl 2-(3-bromo-2-(trifluoromethyl)phenoxy)-7-azaspiro[3.5]nonane-7-carboxylate (2.2 g) as a yellow oil.

LC-MS (ESI): m/z=410.0 [M−56+H]⁺.

### Step 2: 2-(3-bromo-2-(trifluoromethyl)phenoxy)-7-azaspiro[3.5]nonane

A solution of tert-butyl 2-(3-bromo-2-(trifluoromethyl)phenoxy)-7-azaspiro[3.5]nonane-7-carboxylate (2.20 g, 4.74 mmol, 1 equiv.) in HCl/dioxane (2 M, 20 mL) was stirred at 25° C. for 1 hour. The reaction mixture was filtered and concentrated under reduced pressure to afford 2-(3-bromo-2-(trifluoromethyl)phenoxy)-7-azaspiro[3.5]nonane (1.9 g, crude, HCl) as a white solid.

¹H NMR (400 MHz, DMSO-d₆) δ=8.87 (s, 2H), 7.50-7.44 (m, 1H), 7.41-7.36 (m, 1H), 7.08 (d, J=8.4 Hz, 1H), 4.89 (quin, J=6.4 Hz, 1H), 3.56 (s, 3H), 3.06-2.89 (m, 4H), 2.49-2.45 (m, 2H), 1.90-1.71 (m, 6H).

### Step 3: ethyl 2-(2-(3-bromo-2-(trifluoromethyl)phenoxy)-7-azaspiro[3.5]nonan-7-yl)acetate

A mixture of 2-(3-bromo-2-(trifluoromethyl)phenoxy)-7-azaspiro[3.5]nonane (1.9 g, 5.22 mmol, 1 equiv.), ethyl 2-bromoacetate (914.79 mg, 5.48 mmol, 606.22 μL, 1.05 equiv.), and TEA (1.58 g, 15.65 mmol, 2.18 mL, 3 equiv.) in acetone (20 mL) was degassed and purged with N₂ three times. The mixture was stirred at 25° C. for 2 hours under a N₂ atmosphere. The reaction mixture was filtered and concentrated under reduced pressure to give a residue, which was purified by flash silica gel chromatography (0~100% ethyl acetate/petroleum ether) to afford ethyl 2-(2-(3-bromo-2-(trifluoromethyl)phenoxy)-7-azaspiro[3.5]nonan-7-yl)acetate (2.0 g) as a yellow oil.

LC-MS (ESI): m/z=452.2 [M+H]⁺ 1H NMR (400 MHz, DMSO-d₆) δ=7.51-7.32 (m, 2H), 7.07 (d, J=8.4 Hz, 1H), 4.86 (quin, J=6.4 Hz, 1H), 4.06 (q, J=7.2 Hz, 2H), 3.14 (s, 2H), 2.41-2.31 (m, 4H), 1.75 (dd, J=6.0, 12.8 Hz, 2H), 1.55 (td, J=5.2, 16.8 Hz, 4H), 1.17 (t, J=7.2 Hz, 3H).

### Step 4: tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-(trifluoromethyl)phenyl)picolinate

A mixture of ethyl 2-(2-(3-bromo-2-(trifluoromethyl)phenoxy)-7-azaspiro[3.5]nonan-7-yl)acetate (600.00 mg, 1.33 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (979.44 mg, 1.60 mmol, 1.2 equiv.), KF (232.24 mg, 4.00 mmol, 3 equiv.), and Ad₂nBup Pd G₃ (194.08 mg, 266.50 mol, 0.2 equiv.) in dioxane (10 mL) and H₂O (1 mL) was degassed and purged with N₂ three times. The mixture was stirred at 100° C. for 2 hours under a N₂ atmosphere. The reaction mixture was filtered and concentrated under reduced pressure to give a residue, which was purified by flash silica gel chromatography (0~60% ethyl acetate/petroleum ether) to afford tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-(trifluoromethyl)phenyl)picolinate (750 mg) as a yellow solid.

LC-MS (ESI): m/z=856.3 [M+H]⁺

¹H NMR (400 MHz, DMSO-d₆) δ=13.05-12.60 (m, 1H), 8.02 (d, J=7.6 Hz, 1H), 7.78 (d, J=8.0 Hz, 1H), 7.60 (d, J=7.2 Hz, 1H), 7.50-7.32 (m, 6H), 7.02 (d, J=8.4 Hz, 1H), 6.95 (d, J=8.8 Hz, 1H), 6.65 (d, J=8.0 Hz, 1H), 5.06-4.85 (m, 3H), 4.10-4.03 (m, 2H), 3.88 (t, J=5.2 Hz, 2H), 3.19 (s, 2H), 3.02 (t, J=5.6 Hz, 2H), 2.42-2.35 (m, 4H), 1.81-1.65 (m, 4H), 1.61-1.52 (m, 4H), 1.19-1.15 (m, 3H), 1.02 (s, 9H).

### Step 5: 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-(trifluoromethyl)phenyl)picolinate (750.00 mg, 876.21 mol, 1 equiv.), LiOH·H₂O (110.31 mg, 2.63 mmol, 3 equiv.) in THE (6 mL) and H₂O (3 mL) was degassed and purged with N₂ three times. The mixture was stirred at 40° C. for 2 hours under a N₂ atmosphere. The pH of the reaction mixture was adjusted to pH=5-6 by HCl (2 M, 2 mL). The reaction mixture was diluted with H₂O (10 mL), extracted with DCM (15 mL), filtered, and concentrated under reduced pressure to afford 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)-7-azaspiro[3.5]nonan-7-yl)acetic acid (600 mg) as a yellow solid.

LC-MS (ESI): m/z=828.5 [M+H]⁺

### Step 6: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate

A mixture of 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)-7-azaspiro[3.5]nonan-7-yl)acetic acid (120 mg, 144.94 mol, 1 equiv.), 3-(6-amino-1-methyl-indazol-3-yl)piperidine-2,6-dione (48.67 mg, 188.43 mol, 1.3 equiv.), EDCI (41.68 mg, 217.42 mol, 1.5 equiv.) in pyridine (2 mL) was degassed and purged with N₂ three times. The mixture was stirred at 25° C. for 2 hours under a N₂ atmosphere. The reaction mixture was concentrated under reduced pressure to give a residue, which was diluted with H₂O (5 mL) and extracted with DCM (15 mL). The combined organic layers were washed with H₂O (10 mL), dried over sodium sulfate, filtered, and concentrated under reduced pressure to afford tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate (120 mg) as a yellow solid.

LC-MS (ESI): m/z=535.0 [M/2+H]⁺.

### Step 7: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid

A solution of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinate (120.00 mg, 112.34 mol, 1 equiv.) in TFA (0.5 mL) and DCM (0.5 mL) was stirred at 25° C. for 16 hours. The reaction mixture was filtered and concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)oxy)-2-(trifluoromethyl)phenyl)picolinic acid (29.18 mg) as an off-white solid.

LC-MS (ESI): m/z=506.7 [M/2+H]⁺.

¹H NMR (400 MHz, DMSO-d₆) δ=13.21-12.68 (m, 1H), 12.66-12.11 (m, 1H), 10.88 (s, 1H), 9.84 (s, 1H), 8.06-8.00 (m, 2H), 7.79 (d, J=8.0 Hz, 1H), 7.66-7.59 (m, 2H), 7.49-7.32 (m, 6H), 7.22 (dd, J=1.2, 8.8 Hz, 1H), 6.99 (t, J=9.6 Hz, 2H), 6.67 (d, J=7.6 Hz, 1H), 5.06-4.92 (m, 2H), 4.91-4.84 (m, 1H), 4.37-4.28 (m, 1H), 3.96-3.90 (m, 5H), 3.13 (s, 2H), 3.02 (t, J=6.0 Hz, 2H), 2.69-2.58 (m, 2H), 2.48-2.25 (m, 7H), 2.22-2.12 (m, 1H), 1.85-1.75 (m, 2H), 1.74-1.62 (m, 4H).

### Example 50: 6-(8-(benzo[d]thiazol-2-ylcarbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (Compound 171)

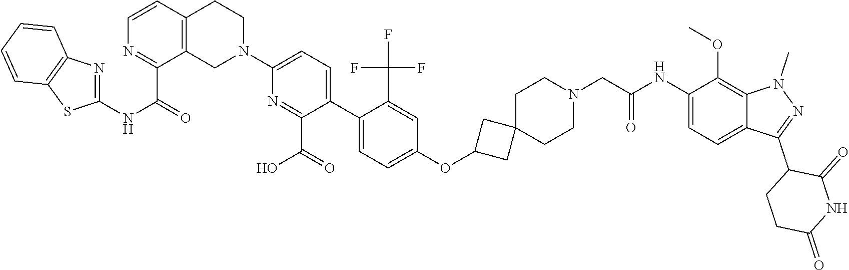 [SMILES: COc1c(NC(=O)CN2CCC3(CC2)CC(Oc2ccc(-c4ccc(N5CCc6ccnc(C(=O)Nc7nc8ccccc8s7)c6C5)nc4C(=O)O)c(C(F)(F)F)c2)C3)ccc2c(C3CCC(=O)NC3=O)nn(C)c12]

### Step 1: 2-(2-(4-bromo-3-(trifluoromethyl)phenoxy)-7-azaspiro[3.5]nonan-7-yl)-N-(3-(2,6-dioxopiperidin-3-yl)-7-methoxy-1-methyl-1H-indazol-6-yl)acetamide

To a solution of 2-[2-[4-bromo-3-(trifluoromethyl)phenoxy]-7-azaspiro[3.5]nonan-7-yl]acetic acid (150 mg, 355.25 mol, 1.0 equiv.) in pyridine (2 mL) was added EDCI (102.15 mg, 532.88 mol, 1.5 equiv.) and 3-(6-amino-7-methoxy-1-methyl-indazol-3-yl)piperidine-2,6-dione (122.90 mg, 426.30 mol, 1.2 equiv.). The mixture was stirred at 40° C. for 1 hour. The reaction mixture was concentrated under reduced pressure to give a residue, which was diluted with H₂O (100 mL) and extracted with DCM (300 mL). The combined organic layers were washed with H₂O (300 mL), dried over sodium sulfate, filtered, and concentrated under reduced pressure to give a residue, which was purified by flash silica gel chromatography (0-10% MeOH/DCM) to give 2-(2-(4-bromo-3-(trifluoromethyl)phenoxy)-7-azaspiro[3.5]nonan-7-yl)-N-(3-(2,6-dioxopiperidin-3-yl)-7-methoxy-1-methyl-1H-indazol-6-yl)acetamide (150 mg) as a yellow solid.

LC-MS (ESI): m/z=694.0 [M+H]⁺

¹H NMR (400 MHz, DMSO-d₆) δ=10.93-10.85 (m, 1H), 9.87-9.75 (m, 1H), 8.01-7.94 (m, 1H), 7.75 (d, J=8.8 Hz, 1H), 7.47-7.43 (m, 1H), 7.23 (d, J=2.8 Hz, 1H), 7.10 (dd, J=2.8, 8.8 Hz, 1H), 4.87 (quin, J=6.8 Hz, 1H), 4.34 (dd, J=5.2, 10.0 Hz, 1H), 4.14 (s, 3H), 3.89 (s, 3H), 3.14 (s, 2H), 2.69-2.60 (m, 2H), 2.57 (d, J=4.4 Hz, 2H), 2.46-2.40 (m, 3H), 2.37-2.24 (m, 2H), 2.20-2.12 (m, 1H), 1.84 (dd, J=6.4, 12.4 Hz, 2H), 1.74 (s, 2H), 1.69 (t, J=4.8 Hz, 2H).

### Step 2: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate

A mixture of 2-(2-(4-bromo-3-(trifluoromethyl)phenoxy)-7-azaspiro[3.5]nonan-7-yl)-N-(3-(2,6-dioxopiperidin-3-yl)-7-methoxy-1-methyl-1H-indazol- 6-yl)acetamide (111.73 mg, 161.34 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (120 mg, 161.34 mol, 1.0 equiv.), KF (28.12 mg, 484.01 mol, 3.0 equiv.), Ad₂nBuP Pd G₃ (58.75 mg, 80.67 mol, 0.5 equiv.), and H₂O (0.3 mL) in dioxane (3 mL) was degassed and purged with N₂ three times. The mixture was stirred at 80° C. for 2 hours under a N₂ atmosphere. The reaction mixture was concentrated under reduced pressure to give a residue, which was diluted with H₂O (20 mL) and extracted with ethyl acetate (60 mL). The combined organic layers was washed with H₂O (20 mL), dried over sodium sulfate, filtered, and concentrated under reduced pressure to give a residue, which was purified by flash silica gel chromatography (0~10% MeOH/DCM) to give tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (60 mg) as a yellow oil.

LC-MS (ESI): m/z=1229.2 [M+H]⁺

### Step 3: 6-(8-(benzo[d]thiazol-2-ylcarbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid

To a solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (60 mg, 48.80 mol, 1.0 equiv.) in TFA (2 mL) was added DCM (1 mL). The mixture was stirred at 40° C. for 1 hour. The reaction mixture was concentrated under reduced pressure to remove solvent. The residue was purified by prep-HPLC to give 6-(8-(benzo[d]thiazol-2-ylcarbamoyl)-3,4-dihydro-2,7-naphthyridin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (23.77 mg) as a yellow solid.

LC-MS (ESI): m/z=1043.2 [M+H]⁺

¹H NMR (400 MHz, DMSO-d₆) δ=12.56-12.48 (m, 1H), 12.31 (s, 1H), 10.89 (s, 1H), 9.87-9.80 (m, 1H), 8.57 (d, J=4.0 Hz, 1H), 8.08-8.03 (m, 1H), 8.02-7.94 (m, 1H), 7.82 (d, J=8.0 Hz, 1H), 7.59 (d, J=4.8 Hz, 1H), 7.53-7.44 (m, 3H), 7.40-7.34 (m, 1H), 7.20-7.07 (m, 4H), 5.35-5.19 (m, 2H), 4.94-4.82 (m, 1H), 4.38-4.30 (m, 1H), 4.14 (s, 3H), 4.06-3.96 (m, 2H), 3.90 (s, 3H), 3.19-3.12 (m, 2H), 3.08 (t, J=4.0 Hz, 2H), 2.70-2.56 (m, 4H), 2.46-2.28 (m, 5H), 2.20-2.13 (m, 1H), 1.90-1.83 (m, 2H), 1.80-1.68 (m, 4H).

### Example 51: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (Compound 172)

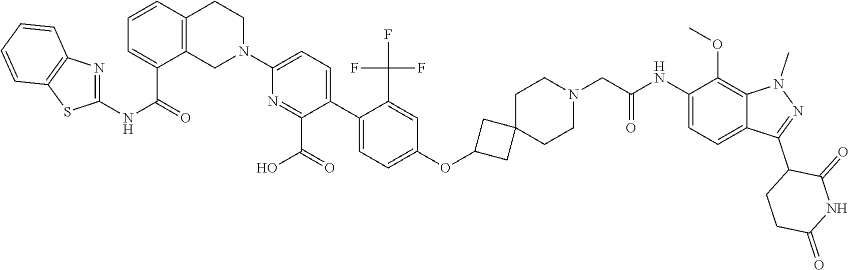 [SMILES: COc1c(NC(=O)CN2CCC3(CC2)CC(Oc2ccc(-c4ccc(N5CCc6cccc(C(=O)Nc7nc8ccccc8s7)c6C5)nc4C(=O)O)c(C(F)(F)F)c2)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate

To a solution of 2-(2-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)-7-azaspiro[3.5]nonan-7-yl)acetic acid (120 mg, 144.94 mol, 1 equiv.) and 3-(6-amino-7-methoxy-1-methyl-indazol-3-yl)piperidine-2,6-dione (62.68 mg, 217.42 mol, 1.5 equiv.) in pyridine (1.2 mL) was added EDCI (41.68 mg, 217.42 mol, 1.5 equiv.). The mixture was stirred at 25° C. for 1 hour. The reaction mixture was partitioned between DCM (15 mL) and H₂O (3 mL). The organic phase was separated, dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (160 mg) as a white solid.

LC-MS (ESI): m/z=550.0 [M/2+H]⁺.

### Step 2: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid

To a solution of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (160 mg, 145.69 mol, 1 equiv.) in DCM (1 mL) was added TFA (2 mL). The mixture was stirred at 25° C. for 12 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (66.85 mg) as a white solid.

LC-MS (ESI): m/z=521.9 [M/2+H]⁺.

¹H NMR (400 MHz, DMSO-d₆) δ=13.25-11.77 (m, 1H), 10.89 (s, 1H), 9.84 (s, 1H), 8.05-7.98 (m, 2H), 7.78 (d, J=8.0 Hz, 1H), 7.63 (d, J=7.2 Hz, 1H), 7.49-7.41 (m, 4H), 7.40-7.32 (m, 2H), 7.20-7.07 (m, 3H), 7.01-6.93 (m, 1H), 5.09-4.93 (m, 2H), 4.87 (q, J=6.8 Hz, 1H), 4.34 (dd, J=5.2, 10.0 Hz, 1H), 4.14 (s, 3H), 3.97-3.88 (m, 5H), 3.14 (s, 3H), 3.03 (t, J=5.2 Hz, 2H), 2.75-2.56 (m, 4H), 2.48-2.29 (m, 4H), 2.22-2.11 (m, 1H), 1.86 (dd, J=6.4, 12.0 Hz, 2H), 1.73 (d, J=17.6 Hz, 4H).

### Example 52: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid (Compound 164)

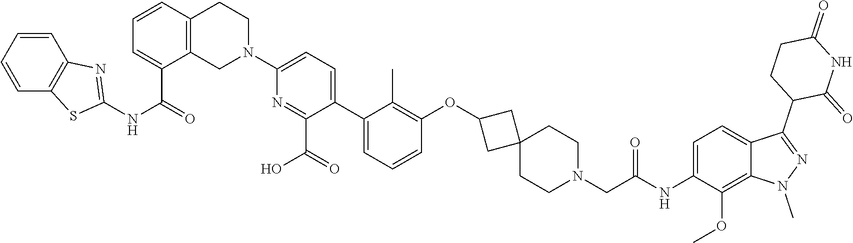 [SMILES: COc1c(NC(=O)CN2CCC3(CC2)CC(Oc2cccc(-c4ccc(N5CCc6cccc(C(=O)Nc7nc8ccccc8s7)c6C5)nc4C(=O)O)c2C)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-methylphenyl)picolinate

A mixture of ethyl 2-[2-(3-bromo-2-methyl-phenoxy)-7-azaspiro[3.5]nonan-7-yl]acetate (300 mg, 756.97 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (556.41 mg, 908.36 mol, 1.2 equiv.), Ad₂nBup Pd G₃ (55.13 mg, 75.70 mol, 0.1 equiv.), and potassium carbonate (1.5 M, 1.51 mL, 3.0 equiv.) in dioxane (10 mL) was degassed and purged with N₂ three times. The mixture was stirred at 100° C. for 1 hour under a N₂ atmosphere in a microwave reactor. The mixture was concentrated under reduced pressure to give a residue, which was purified by flash silica gel chromatography (0~70% ethyl acetate/petroleum ether) to afford tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-methylphenyl)picolinate (460 mg) as a yellow solid.

LC-MS (ESI): m/z=802.3 [M+H]⁺

### Step 2: 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)oxy)-2-methylphenyl)picolinate (460 mg, 573.57 mol, 1.0 equiv.) and LiOH H₂O (72.21 mg, 1.72 mmol, 3.0 equiv.) in THE (5 mL) and H₂O (1 mL) was stirred at 40° C. for 2 hours. The reaction mixture was diluted with H₂O (5 mL) and concentrated as a turbid liquid. The pH of the turbid liquid was adjusted to pH=3 with 1 N HCl (5 mL) and extracted with DCM/MeOH (10:1). The combined organic layers were filtered and concentrated to afford 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-7-azaspiro[3.5]nonan-7-yl)acetic acid (420 mg) as a yellow solid.

LC-MS (ESI): m/z=774.1 [M+H]⁺

### Step 3: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate

A mixture of 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-7-azaspiro[3.5]nonan-7-yl)acetic acid (100 mg, 129.21 mol, 1.0 equiv.), 3-(6-amino-7-methoxy-1-methyl-indazol-3-yl)piperidine-2,6-dione (48.43 mg, 167.97 mol, 1.3 equiv.), and EDCI (37.15 mg, 193.81 mol, 1.5 equiv.) in pyridine (2 mL) was degassed. The mixture was stirred at 40° C. for 2 hours. The reaction mixture was diluted with H₂O (5 mL) and concentrated as a turbid liquid. The residue was extracted with ethyl acetate (15 mL) and the combined organic layers were filtered and concentrated to give a residue, which was purified by flash silica gel chromatography (0~10% methanol/dichloromethane) to afford tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate (130 mg) as a brown solid.

LC-MS (ESI): m/z=1044.2 [M+H]⁺

### Step 4: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinate (130 mg, 124.49 mol, 1.0 equiv.) in TFA (1.5 mL) and DCM (1.5 mL) was stirred at 25° C. for 2 hours. The mixture was concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-methylphenyl)picolinic acid (58.26 mg) as an off-white solid.

LC-MS (ESI): m/z=988.3 [M+H]⁺

¹H NMR (400 MHz, DMSO-d₆) δ=13.03-12.36 (m, 2H), 10.89 (s, 1H), 10.00-9.62 (m, 1H), 8.10-7.91 (m, 2H), 7.81-7.77 (m, 1H), 7.62 (d, J=6.8 Hz, 1H), 7.48-7.43 (m, 4H), 7.40-7.34 (m, 2H), 6.93 (dd, J=8.8, 16.4 Hz, 2H), 6.71 (d, J=2.8 Hz, 1H), 6.63 (dd, J=2.8, 8.0 Hz, 1H), 4.97 (s, 2H), 4.77-4.70 (m, 1H), 4.34 (dd, J=5.2, 10.0 Hz, 1H), 4.14 (s, 3H), 3.93-3.87 (m, 5H), 3.48 (d, J=11.6 Hz, 2H), 3.14 (d, J=2.4 Hz, 2H), 3.02 (t, J=5.2 Hz, 2H), 2.71-2.56 (m, 4H), 2.47-2.39 (m, 2H), 2.38-2.37 (m, 1H), 2.38-2.29 (m, 1H), 2.20-2.14 (m, 1H), 2.03 (s, 3H), 1.92-1.79 (m, 3H), 1.77-1.67 (m, 3H).

### Example 53: 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (Compound 165)

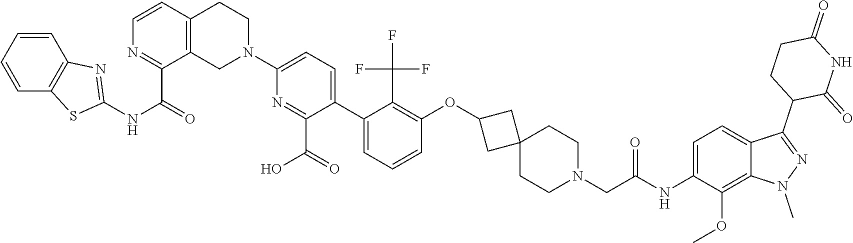 [SMILES: COc1c(NC(=O)CN2CCC3(CC2)CC(Oc2cccc(-c4ccc(N5CCc6ccnc(C(=O)Nc7nc8ccccc8s7)c6C5)nc4C(=O)O)c2C(F)(F)F)C3)ccc2c(C3CCC(=O)NC3=O)nn(C)c12]

### Step 1: 2-(2-(3-bromo-2-(trifluoromethyl)phenoxy)-7-azaspiro[3.5]nonan-7-yl)-N-(3-(2,6-dioxopiperidin-3-yl)-7-methoxy-1-methyl-1H-indazol-6-yl)acetamide

To a solution of 2-[2-[3-bromo-2-(trifluoromethyl)phenoxy]-7-azaspiro[3.5]nonan-7-yl]acetic acid (150 mg, 355.25 mol, 1.0 equiv.) in pyridine (2 mL) was added EDCI (102.15 mg, 532.88 mol, 1.5 equiv.) and 3-(6-amino-7-methoxy-1-methyl-indazol-3-yl)piperidine-2,6-dione (122.90 mg, 426.30 mol, 1.2 equiv.). The mixture was stirred at 40° C. for 1 hour. The reaction mixture was concentrated under reduced pressure to give a residue, which was diluted with H₂O (30 mL) and extracted with DCM (100 mL). The combined organic layers was washed with H₂O (90 mL), dried over sodium sulfate, filtered, and concentrated under reduced pressure to give a residue, which was purified by flash silica gel chromatography (0-10% MeOH/DCM) to give 2-(2-(3-bromo-2-(trifluoromethyl)phenoxy)-7-azaspiro[3.5]nonan-7-yl)-N-(3-(2,6-dioxopiperidin-3-yl)-7-methoxy-1-methyl-1H-indazol-6-yl)acetamide (90 mg) as a yellow solid.

LC-MS (ESI): m/z=692.0 [M+H]⁺

### Step 2: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate

A mixture of 2-(2-(3-bromo-2-(trifluoromethyl)phenoxy)-7-azaspiro[3.5]nonan-7-yl)-N-(3-(2,6-dioxopiperidin-3-yl)-7-methoxy-1-methyl-1H-indazol-6-yl)acetamide (90 mg, 129.96 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (96.66 mg, 129.96 mol, 1.0 equiv.), KF (22.65 mg, 389.88 mol, 3.0 equiv.), Ad₂nBuP Pd G₃ (47.32 mg, 64.98 mol, 0.5 equiv.), and H₂O (0.2 mL) in dioxane (2 mL) was degassed and purged with N₂ three times. The mixture was stirred at 80° C. for 2 hours under a N₂ atmosphere. The reaction mixture was concentrated under reduced pressure to give a residue, which was diluted with H₂O (20 mL) and extracted with ethyl acetate (60 mL). The combined organic layers were washed with H₂O (20 mL), dried over sodium sulfate, filtered, and concentrated under reduced pressure to give a residue, which was purified by flash silicagel chromatography (0~10% MeOH/DCM) to give tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (100 mg) as a white solid.

LC-MS (ESI): m/z=1229.5 [M+H]⁺

### Step 3: 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid

A solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (100 mg, 81.34 μmol, 1.0 equiv.) in TFA (2 mL) and DCM (1 mL) was stirred at 40° C. for 1 hour. The reaction mixture was concentrated under reduced pressure to give a residue, which was purified by prep-HPLC to give 6-(8-(benzo[d]thiazol-2-ylcarbamoyl)-3,4-dihydro-2,7-naphthyridin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (19.29 mg) as a white solid.

LC-MS (ESI): m/z=1043.2 [M+H]⁺

¹H NMR (400 MHz, DMSO-d₆) δ=12.58-12.53 (m, 1H), 12.31 (s, 1H), 10.87 (s, 1H), 9.86-9.80 (m, 1H), 8.60-8.54 (m, 1H), 8.05 (d, J=7.6 Hz, 1H), 8.02-7.96 (m, 1H), 7.81 (d, J=7.6 Hz, 1H), 7.59 (d, J=5.2 Hz, 1H), 7.52-7.44 (m, 4H), 7.40-7.34 (m, 1H), 7.06 (dd, J=8.8, 18.4 Hz, 2H), 6.70 (d, J=7.6 Hz, 1H), 5.33-5.18 (m, 2H), 4.97-4.86 (m, 1H), 4.38-4.30 (m, 1H), 4.14 (s, 3H), 4.05-3.96 (m, 2H), 3.89 (s, 3H), 3.15 (s, 2H), 3.08 (t, J=5.6 Hz, 2H), 2.69-2.56 (m, 4H), 2.48-2.29 (m, 5H), 2.21-2.12 (m, 1H), 1.89-1.81 (m, 2H), 1.73 (d, J=17.6 Hz, 4H).

### Example 54: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (Compound 166)

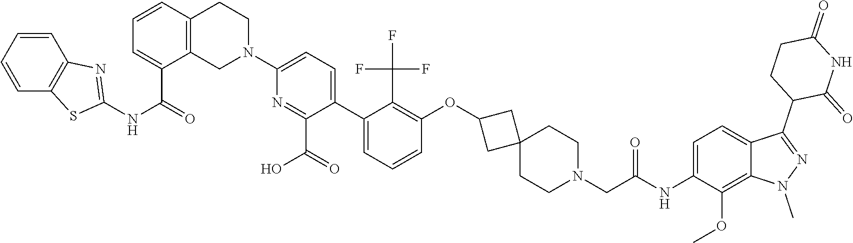 [SMILES: COc1c(NC(=O)CN2CCC3(CC2)CC(Oc2cccc(-c4ccc(N5CCc6cccc(C(=O)Nc7nc8ccccc8s7)c6C5)nc4C(=O)O)c2C(F)(F)F)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate

A mixture of 2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)-7-azaspiro[3.5]nonan-7-yl)acetic acid (120 mg, 144.94 mol, 1 equiv.), 3-(6-amino-7-methoxy-1-methyl-indazol-3-yl)piperidine-2,6-dione (54.32 mg, 188.43 mol, 1.3 equiv.), and EDCI (41.68 mg, 217.42 mol, 1.5 equiv.) in pyridine (2 mL) was degassed and purged with N₂ three times. The mixture was stirred at 25° C. for 2 hours under a N₂ atmosphere. The reaction mixture was concentrated under reduced pressure to give a residue, which was diluted with H₂O (5 mL) and extracted with DCM (15 mL). The combined organic layers were washed with brine (10 mL), dried over sodium sulfate, filtered, and concentrated under reduced pressure to afford tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (120 mg) as a yellow solid.

LC-MS (ESI): m/z=550.0 [M/2+H]⁺.

### Step 2: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid

A solution of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinate (120.00 mg, 109.27 mol, 1 equiv.) in TFA (0.5 mL) and DCM (0.5 mL) was stirred at 25° C. for 16 hours. The reaction mixture was filtered and concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)oxy)-2-(trifluoromethyl)phenyl)picolinic acid (45.54 mg) as a white solid.

LC-MS (ESI): m/z=521.9 [M/2+H]⁺.

¹H NMR (400 MHz, DMSO-d₆) δ=13.00-12.37 (m, 1H), 10.89 (s, 1H), 9.86-9.80 (m, 1H), 8.01 (dd, J=8.4, 12.0 Hz, 2H), 7.78 (d, J=8.0 Hz, 1H), 7.62 (d, J=7.2 Hz, 1H), 7.50-7.31 (m, 7H), 7.05-6.93 (m, 2H), 6.68 (d, J=7.6 Hz, 1H), 5.06-4.85 (m, 3H), 4.34 (dd, J=4.8, 10.0 Hz, 1H), 4.14 (s, 3H), 3.93 (t, J=5.6 Hz, 2H), 3.89 (s, 3H), 3.14 (s, 2H), 3.02 (t, J=5.6 Hz, 2H), 2.68-2.55 (m, 4H), 2.48-2.42 (m, 3H), 2.42-2.26 (m, 2H), 2.21-2.13 (m, 1H), 1.88-1.78 (m, 2H), 1.71 (d, J=18.4 Hz, 4H).

### Example 55: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (Compound 167)

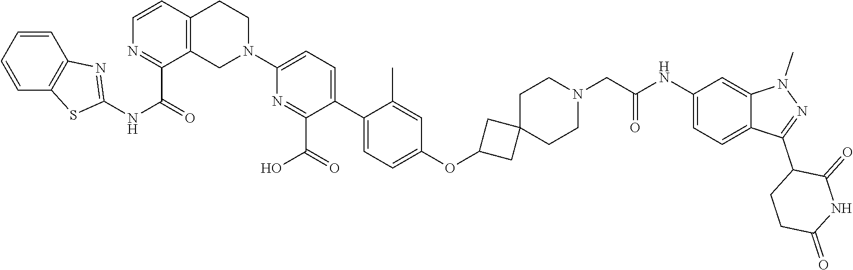 [SMILES: Cc1cc(OC2CC3(CCN(CC(=O)Nc4ccc5c(C6CCC(=O)NC6=O)nn(C)c5c4)CC3)C2)ccc1-c1ccc(N2CCc3ccnc(C(=O)Nc4nc5ccccc5s4)c3C2)nc1C(=O)O]

### Step 1: 2-[2-(4-bromo-3-methyl-phenoxy)-7-azaspiro[3.5]nonan-7-yl]acetic acid

A mixture of ethyl 2-[2-(4-bromo-3-methyl-phenoxy)-7-azaspiro[3.5]nonan-7-yl]acetate (0.50 g, 1.26 mmol, 1.0 equiv.), LiOH·H₂O (211.77 mg, 5.05 mmol, 4.0 equiv.) in THE (8 mL) and H₂O (2 mL) was degassed and purged with N₂ three times. The mixture was stirred at 40° C. for 2 hours under a N₂ atmosphere. The reaction mixture was concentrated under reduced pressure to give a residue, which was adjusted to pH=3-4 with HCl and extracted with EtOAc (300 mL). The combined organic layers were washed with brine (300 mL), dried over sodium sulfate, filtered, and concentrated under reduced pressure to give 2-[2-(4-bromo-3-methyl-phenoxy)-7-azaspiro[3.5]nonan-7-yl]acetic acid (0.45 g) as a yellow oil.

LC-MS (ESI): m/z=368.0 [M+H]⁺

### Step 2: 2-[2-(4-bromo-3-methyl-phenoxy)-7-azaspiro[3.5]nonan-7-yl]-N-[3-(2,6-dioxo-3-piperidyl)-1-methyl-indazol-6-yl]acetamide

A mixture of 2-[2-(4-bromo-3-methyl-phenoxy)-7-azaspiro[3.5]nonan-7-yl]acetic acid (0.30 g, 814.63 mol, 1.0 equiv.), 3-(6-amino-1-methyl-indazol-3-yl)piperidine-2,6-dione (252.48 mg, 977.56 mol, 1.2 equiv.), and EDCI (234.25 mg, 1.22 mmol, 1.5 equiv.) in pyridine (10 mL) was degassed and purged with N₂ three times. The mixture was stirred at 40° C. for 2 hours under a N₂ atmosphere. The reaction mixture was concentrated under reduced pressure to give a residue, which was purified by flash silica gel chromatography (0~100% ethyl acetate/petroleum ether) to give 2-[2-(4-bromo-3-methyl-phenoxy)-7-azaspiro[3.5]nonan-7-yl]-N-[3-(2,6-dioxo-3-piperidyl)-1-methyl-indazol-6-yl]acetamide (0.30 g) as a yellow solid.

LC-MS (ESI): m/z=608.1 [M+H]⁺

### Step 3: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate

A mixture of 2-[2-(4-bromo-3-methyl-phenoxy)-7-azaspiro[3.5]nonan-7-yl]-N-[3-(2,6-dioxo-3-piperidyl)-1-methyl-indazol-6-yl]acetamide (90.0 mg, 147.90 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (132.01 mg, 177.48 mol, 1.2 equiv.), Ad₂nBuP Pd G₃ (53.86 mg, 73.95 mol, 0.5 equiv.), and KF (25.78 mg, 443.70 mol, 3.0 equiv.) in dioxane (10 mL) and H₂O (1 mL) was degassed and purged with N₂ three times. The mixture was stirred at 80° C. for 12 hours under a N₂ atmosphere. The reaction mixture was concentrated under reduced pressure to give a residue, which was purified by flash silica gel chromatography (0-90% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (96 mg) as a yellow solid.

LC-MS (ESI): m/z=573.5 [M/2+H]⁺.

### Step 4: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid

A mixture of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylate (96 mg, 83.81 mol, 1.0 equiv.) in TFA (2 mL) and DCM (2 mL) was stirred at 40° C. for 2 hours. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-1-methyl-indazol-6-yl]amino]-2-oxo-ethyl]-7-azaspiro[3.5]nonan-2-yl]oxy]-2-methyl-phenyl]pyridine-2-carboxylic acid (13.69 mg) as a yellow solid.

LC-MS (ESI): m/z=959.3 [M+H]⁻

¹H NMR (400 MHz, DMSO-d₆) δ=10.87 (s, 1H), 9.84 (s, 1H), 8.57 (d, J=4.8 Hz, 1H), 8.07-8.03 (m, 2H), 7.82 (d, J=8.0 Hz, 1H), 7.63 (d, J=8.4 Hz, 1H), 7.58 (d, J=4.8 Hz, 1H), 7.52-7.44 (m, 2H), 7.40-7.34 (m, 1H), 7.25-7.20 (m, 1H), 7.06-6.99 (m, 1H), 6.93 (d, J=8.0 Hz, 1H), 6.71 (d, J=1.6 Hz, 1H), 6.66-6.60 (m, 1H), 5.24 (s, 2H), 4.77-4.68 (m, 1H), 4.32 (dd, J=4.8, 9.6 Hz, 1H), 4.00-3.94 (m, 2H), 3.92 (s, 3H), 3.12 (s, 2H), 3.07 (t, J=5.2 Hz, 2H), 2.69-2.54 (m, 4H), 2.45-2.39 (m, 4H), 2.33 (d, J=1.6 Hz, 1H), 2.22-2.13 (m, 1H), 2.04 (s, 3H), 1.84-1.75 (m, 2H), 1.74-1.61 (m, 4H).

### Example 56: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid (Compound 187)

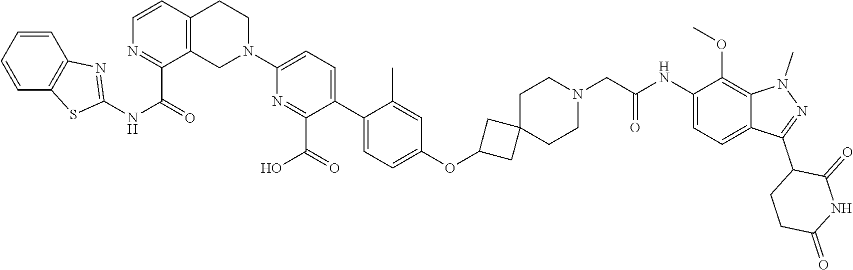 [SMILES: COc1c(NC(=O)CN2CCC3(CC2)CC(Oc2ccc(-c4ccc(N5CCc6ccnc(C(=O)Nc7nc8ccccc8s7)c6C5)nc4C(=O)O)c(C)c2)C3)ccc2c(C3CCC(=O)NC3=O)nn(C)c12]

### Step 1: 2-[2-(4-bromo-3-methyl-phenoxy)-7-azaspiro[3.5]nonan-7-yl]-N-[3-(2,6-dioxo-3-piperidyl)-7-methoxy-1-methyl-indazol-6-yl]acetamide

To a solution of 2-[2-(4-bromo-3-methyl-phenoxy)-7-azaspiro[3.5]nonan-7-yl]acetic acid (200 mg, 543.09 mol, 1.0 equiv.) and 3-(6-amino-7-methoxy-1-methyl-indazol-3-yl)piperidine-2,6-dione (187.89 mg, 578.54 mol, 1.07 equiv., HCl) in pyridine (4 mL) was added EDCI (104.11 mg, 543.09 mol, 1.0 equiv.). The mixture was stirred at 25° C. for 1 hour. The residue was diluted with water (100 mL) and extracted by ethyl acetate (100 mL). The combined organic layers were washed with brine (60 mL), dried over anhydrous sodium sulfate, filtered, and concentrated under vacuum. The residue was purified by flash silica gel chromatography (0~10% MeOH/DCM) to give 2-[2-(4-bromo-3-methyl-phenoxy)-7-azaspiro[3.5]nonan-7-yl]-N-[3-(2,6-dioxo-3-piperidyl)-7-methoxy-1-methyl-indazol-6-yl]acetamide (200 mg) as a yellow solid.

LC-MS (ESI): m/z=638.2 [M+H]⁺

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate

A mixture of 2-[2-(4-bromo-3-methyl-phenoxy)-7-azaspiro[3.5]nonan-7-yl]-N-[3-(2,6-dioxo-3-piperidyl)-7-methoxy-1-methyl-indazol-6-yl]acetamide (80 mg, 125.28 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (139.78 mg, 150.34 mol, 1.2 equiv.), H₂O (0.2 mL), KF (21.84 mg, 375.85 mol, 3.0 equiv.), and Ad₂nBuP Pd G₃ (45.62 mg, 62.64 mol, 0.5 equiv.) in dioxane (2 mL) was degassed and purged with N₂ three times. The mixture was stirred at 80° C. for 12 hours under a N₂ atmosphere. The reaction mixture was concentrated under reduced pressure to give a residue, which was purified by flash silica gel chromatography (0~100% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate (100 mg) as a yellow solid.

LC-MS (ESI): m/z=588.6 [M/2+H]⁺.

### Step 3: 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid

To a solution of tert-butyl 6-[8-[1,3-benzothiazol-2-yl(2-trimethylsilylethoxymethyl)carbamoyl]-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate (100 mg, 34.03 mol, 1.0 equiv.) in DCM (1 mL) was added TFA (1 mL). The mixture was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[4-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid (23.27 mg) as a yellow solid.

LC-MS (ESI): m/z=495.4 [M/2+H]⁺.

¹H NMR (400 MHz, DMSO-d₆) δ=12.72-12.64 (m, 1H), 12.35 (s, 1H), 10.92 (s, 1H), 8.57 (d, J=4.8 Hz, 1H), 8.05 (d, J=8.0 Hz, 1H), 7.82 (d, J=8.0 Hz, 1H), 7.59 (d, J=4.8 Hz, 1H), 7.51-7.35 (m, 5H), 7.05 (d, J=8.8 Hz, 1H), 6.94 (d, J=8.4 Hz, 1H), 6.73 (d, J=2.4 Hz, 1H), 6.65 (d, J=8.4 Hz, 1H), 5.24 (s, 2H), 4.79-4.70 (m, 1H), 4.39-4.31 (m, 1H), 4.29-4.22 (m, 1H), 4.15 (s, 3H), 3.97 (t, J=5.2 Hz, 2H), 3.91-3.85 (m, 3H), 3.45-3.44 (m, 2H), 3.15-3.13 (m, 1H), 3.09-3.06 (m, 2H), 2.60 (d, J=5.2 Hz, 2H), 2.45-2.41 (m, 2H), 2.21-2.11 (m, 2H), 2.05 (s, 3H), 1.97-1.79 (m, 6H), 1.76-1.70 (m, 2H)

### Example 57: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid (Compound 184)

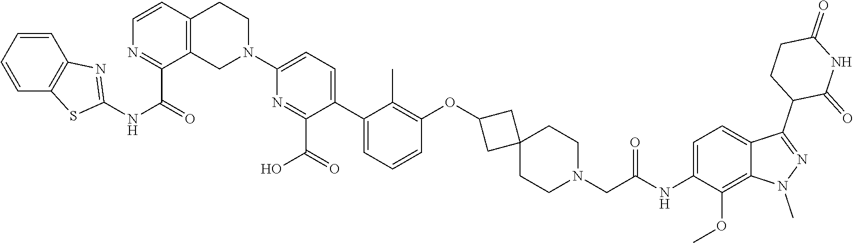 [SMILES: COc1c(NC(=O)CN2CCC3(CC2)CC(Oc2cccc(-c4ccc(N5CCc6ccnc(C(=O)Nc7nc8ccccc8s7)c6C5)nc4C(=O)O)c2C)C3)ccc2c(C3CCC(=O)NC3=O)nn(C)c12]

### Step 1: 2-[2-(3-bromo-2-methyl-phenoxy)-7-azaspiro[3.5]nonan-7-yl]-N-[3-(2,6-dioxo-3-piperidyl)-7-methoxy-1-methyl-indazol-6-yl]acetamide

A mixture of 2-[2-(3-bromo-2-methyl-phenoxy)-7-azaspiro[3.5]nonan-7-yl]acetic acid (0.3 g, 814.63 mol, 1.0 equiv.), 3-(6-amino-7-methoxy-1-methyl-indazol-3-yl)piperidine-2,6-dione (281.83 mg, 977.56 mol, 1.2 equiv.), and EDCI (234.25 mg, 1.22 mmol, 1.5 equiv.) in pyridine (5 mL) was stirred at 40° C. for 2 hours. The reaction mixture was concentrated under reduced pressure to give a residue, which was purified by flash silica gel chromatography (0~100% ethyl acetate/petroleum ether) to give 2-[2-(3-bromo-2-methyl-phenoxy)-7-azaspiro[3.5]nonan-7-yl]-N-[3-(2,6-dioxo-3-piperidyl)-7-methoxy-1-methyl-indazol-6-yl]acetamide (0.3 g) as a yellow solid.

LC-MS (ESI): m/z=638.2 [M+H]⁺

### Step 2: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate

A mixture of 2-[2-(3-bromo-2-methyl-phenoxy)-7-azaspiro[3.5]nonan-7-yl]-N-[3-(2,6-dioxo-3-piperidyl)-7-methoxy-1-methyl-indazol-6-yl]acetamide (90.0 mg, 140.94 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (125.80 mg, 169.13 mol, 1.2 equiv.), Ad₂nBuP Pd G₃ (51.32 mg, 70.47 mol, 0.5 equiv.), and KF (24.57 mg, 422.83 mol, 3.0 equiv.) in dioxane (5 mL) and H₂O (0.5 mL) was degassed and purged with N₂ three times. The mixture was stirred at 80° C. for 2 hours under a N₂ atmosphere. The reaction mixture was concentrated under reduced pressure to give a residue, which was purified by flash silica gel chromatography (0~100% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate (180.0 mg) as a yellow oil.

LC-MS (ESI): m/z=588.7 [M/2+H]⁺.

### Step 3: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid

A mixture of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylate (106.0 mg, 90.18 mol, 1.0 equiv.) in TFA (6 mL) and DCM (6 mL) was stirred at 40° C. for 2 hours under a N₂ atmosphere. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-7-methoxy-1-methyl-indazol-6-yl]amino]-2-oxo-ethyl]-7-azaspiro[3.5]nonan-2-yl]oxy]-2-methyl-phenyl]pyridine-2-carboxylic acid (15.71 mg) as a yellow solid.

LC-MS (ESI): m/z=989.3 [M+H]⁺

¹H NMR (400 MHz, DMSO-d₆) δ=10.89 (s, 1H), 9.84 (s, 1H), 8.56 (d, J=5.2 Hz, 1H), 8.03 (dd, J=8.4, 19.6 Hz, 2H), 7.81 (d, J=8.0 Hz, 1H), 7.58 (d, J=4.8 Hz, 1H), 7.51-7.43 (m, 3H), 7.39-7.33 (m, 1H), 7.11-6.99 (m, 2H), 6.75-6.64 (m, 2H), 5.24 (s, 2H), 4.78-4.71 (m, 1H), 4.34 (dd, J=4.8, 10.0 Hz, 1H), 4.14 (s, 3H), 4.00-3.94 (m, 2H), 3.90 (s, 3H), 3.14 (s, 2H), 3.07 (t, J=5.6 Hz, 2H), 2.69-2.54 (m, 6H), 2.36-2.29 (m, 2H), 2.25-2.10 (m, 2H), 1.92 (s, 3H), 1.88-1.82 (m, 2H), 1.76-1.69 (m, 4H).

### Example 58. 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinic acid (Compound 122)

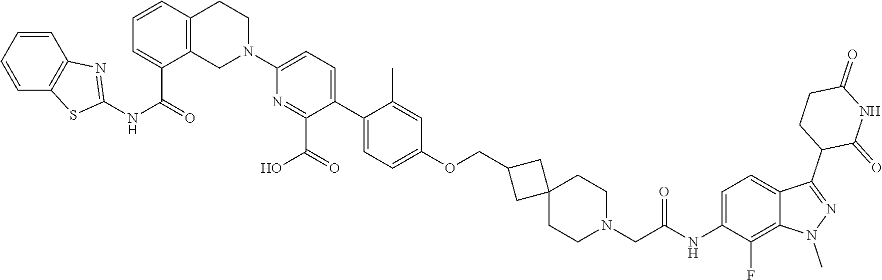 [SMILES: Cc1cc(OCC2CC3(CCN(CC(=O)Nc4ccc5c(C6CCC(=O)NC6=O)nn(C)c5c4F)CC3)C2)ccc1-c1ccc(N2CCc3cccc(C(=O)Nc4nc5ccccc5s4)c3C2)nc1C(=O)O]

### Step 1: 6-bromo-7-fluoro-1H-indazole

A solution of 4-bromo-2,3-difluorobenzaldehyde (25 g, 113.12 mmol), O-methylhydroxylamine (28.34 g, 339.36 mmol) and potassium carbonate (23.45 g, 169.68 mmol) in DCE (250 mL) was stirred at room temperature for 8 hours. To this solution was added hydrazine (125.0 mL) at 0° C. The reaction mixture was heated at 100° C. for 40 hours. The reaction mixture was quenched with ice cold water (300 mL) and extracted with ethyl-acetate (500 mL). The separated organic layer was washed with brine solution (50 mL), dried over anhydrous sodium sulphate, filtered, and concentrated to get crude product, which was purified by trituration with diethyl-ether (200 mL) to afford 6-bromo-7-fluoro-1H-indazole (16.0 g) as an off white solid.

LC-MS (ESI): m/z=212.95 [M−H]⁻

### Step 2: 6-bromo-7-fluoro-3-iodo-1H-indazole

A solution of 6-bromo-7-fluoro-1H-indazole (16.0 g, 74.41 mmol) and potassium hydroxide (12.52 g, 223.23 mmol) in THE (160 mL) was stirred at room temperature for 1 hour. To this solution was added iodine (18.88 g, 74.41 mmol) at 0° C. The reaction mixture was stirred at room temperature for 16 hours. The reaction mixture was quenched by ice-water (200 ml) and extracted with ethyl-acetate (500 mL). The organic layer was concentrated under reduced pressure to get crude product, which was purified by trituration with diethyl-ether (200 mL) to get 6-bromo-7-fluoro-3-iodo-1H-indazole (20.0 g) as a brown solid.

LC-MS (ESI): m/z=342.40 [M+H]⁺

### Step 3: 6-bromo-7-fluoro-3-iodo-1-methyl-1H-indazole

A solution of 6-bromo-7-fluoro-3-iodo-1H-indazole (20.0 g, 58.66 mmol) and potassium tert-butoxide (19.75 g, 175.99 mmol) in THE (200 mL) was stirred for 10 min at room temperature. To this solution was added iodomethane (8.32 g, 58.66 mmol) at 0° C. The reaction mixture was stirred at room temperature for 3 hours. The reaction mixture was quenched by ice-water (200 mL) and extracted with ethyl-acetate (200 mL). The organic layer was concentrated under reduced pressure to get crude product, which was purified by flash column chromatography (5-10% ethyl-acetate in petroleum ether) to afford 6-bromo-7-fluoro-3-iodo-1-methyl-1H-indazole (9.0 g).

LC-MS (ESI): m/z=354.84 [M+H]⁺

### Step 4: 3-(2,6-bis(benzyloxy)pyridin-3-yl)-6-bromo-7-fluoro-1-methyl-1H-indazole

To a stirred solution of 6-bromo-7-fluoro-3-iodo-1-methyl-1H-indazole (5.0 g, 14.08 mmol), 2,6-bis(benzyloxy)pyridin-3-yl)boronic-acid (4.72 g, 14.08 mmol) and cesium carbonate (11.47 g, 35.21 mmol) in dioxane (50 mL) under nitrogen gas was added XantPhos (1.63 g, 2.81 mmol) and Pd₂(dba)₃ (1.29 g, 1.40 mmol). The mixture was heated at 100° C. and stirred for 16 hours. The reaction mixture was filtered through a celite pad and reaction mixture was quenched by ice-water (50 mL) and extracted with ethyl-acetate (200 mL). The organic layer was concentrated under reduced pressure to get crude product which was purified by flash column chromatography (0-20% ethyl-acetate in petroleum ether) to afford 3-(2,6-bis(benzyloxy)pyridin-3-yl)-6-bromo-7-fluoro-1-methyl-1H-indazole (3 g) as a white solid.

LC-MS (ESI): m/z=520.36 [M+H]⁺

### Step 5: N-benzyl-3-(2,6-bis(benzyloxy)pyridin-3-yl)-7-fluoro-1-methyl-1H-indazol-6-amine

A solution of 3-(2,6-bis(benzyloxy)pyridin-3-yl)-6-bromo-7-fluoro-1-methyl-1H-indazole (15.0 g, 28.96 mmol), phenylmethanamine (6.2 g, 57.00 mmol) and t-BuONa (8.3 g, 86.80 mmol) in dioxane (150 mL) was purged with argon for 15 min. To this mixture, BrettPhos-Pd G3 (21.3 g, 1.44 mmol) was added. The reaction mixture was heated to 100° C. for 16 hours. The reaction mixture was filtered through a celite bed and concentrated under reduced pressure. The residue was diluted with water (500 mL) and extracted ethyl acetate (1500 mL). The combined organic layer was washed with brine (500 mL), dried over anhydrous sodium sulfate, filtered and concentrated under reduced pressure. The crude product was purified by silica gel column chromatography (0-15% ethyl acetate and petroleum ether) to afford N-benzyl-3-(2,6-bis(benzyloxy)pyridin-3-yl)-7-fluoro-1-methyl-1H-indazol-6-amine (8.0 g) as an off-white solid.

LC-MS (ESI): m/z=245.60 [M+H]⁺

¹H NMR (400 MHz, DMSO-d₆): δ 7.82 (d, J=8 Hz, 1H), 7.45 (d, J=6.8 Hz, 2H), 7.40-7.31 (m, 9H), 7.28-7.13 (m, 5H), 6.55-6.47 (m, 2H), 6.33 (s, 1H), 5.39 (d, J=5.6 Hz, 4H), 4.43 (d, J=6.4 Hz, 2H), 4.08 (s, 3H).

### Step 6: 3-(6-amino-7-fluoro-1-methyl-1H-indazol-3-yl)piperidine-2,6-dione

A solution of N-benzyl-3-(2,6-bis(benzyloxy)pyridin-3-yl)-7-fluoro-1-methyl-1H-indazol-6-amine (8.0 g, 14.69 mmol) in THF:methanol (200 mL; 4:1) was purged with argon for 5 min followed by the addition of Pd on carbon (10 wt %, 8.0 g). The mixture was stirred under H₂ (50 psi) at room temperature for 7 hours. The reaction mixture was filtered through a celite bed and washed with THE (200 mL). The combined filtrate was concentrated under reduced pressure. The residue was purified by reverse phase flash column chromatography (25-30% water with 0.1% formic acid in acetonitrile) to afford 3-(6-amino-7-fluoro-1-methyl-1H-indazol-3-yl)piperidine-2,6-dione (1.0 g) as an off-white solid.

LC-MS (ESI): m/z=277.26 [M+H]⁺

¹H NMR (400 MHz, DMSO-d₆): δ 10.85 (s, 1H), 7.17 (d, J=8.8 Hz, 1H), 6.64-6.61 (m, 1H), 5.31 (s, 2H), 4.24-4.20 (m, 1H), 3.99 (s, 3H), 2.64-2.59 (m, 2H), 2.29-2.25 (m, 1H), 2.16-2.11 (m, 1H).

### Step 7: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinate

2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl]acetic acid (100 mg, 1 equiv., 127 mol) was suspended in DCM (5 mL). To this mixture was added HATU (53.1 mg, 1.1 equiv., 140 mol), followed by 3-(6-amino-7-fluoro-1-methyl-1H-indazol-3-yl)piperidine-2,6-dione (42.1 mg, 1.2 equiv., 152 mol) and DIPEA (49.2 mg, 3 equiv., 381 mol). The mixture was heated at 40° C. The mixture was then concentrated and extracted with ethyl acetate and water. The organic layer was separated, washed with brine, dried over sodium sulfate, filtered, and concentrated under reduced pressure to afford tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinate, which was used in the next step without further purification.

LC-MS (ESI): m/z=523.9 [M+2H]2+

### Step 8: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinic acid

tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinate (133 mg, 1 equiv., 127 mol) was dissolved in DCM (4 mL) and TFA (1 mL). The mixture was then stirred at room temperature overnight. The mixture was then concentrated under reduced pressure and purified by HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-fluoro-1-methyl-1H-indazol-6-yl)amino)-2-oxoethyl)-7-azaspiro[3.5]nonan-2-yl)methoxy)-2-methylphenyl)picolinic acid (36 mg) as a white solid.

LC-MS (ESI): m/z=495.9 [M+2H]²⁺

¹H NMR (400 MHz, DMSO-d₆) δ 12.79 (s, 1H), 12.48 (s, 1H), 10.85 (s, 1H), 7.96 (d, J=7.9 Hz, 1H), 7.72 (d, J=8.0 Hz, 1H), 7.55 (d, J=7.5 Hz, 1H), 7.47-7.34 (m, 5H), 7.34-7.24 (m, 2H), 6.87 (dd, J=16.3, 8.6 Hz, 2H), 6.73 (d, J=2.6 Hz, 1H), 6.64 (dd, J=8.4, 2.6 Hz, 1H), 4.90 (s, 2H), 4.31 (dd, J=10.4, 5.0 Hz, 1H), 4.05 (s, 3H), 3.90-3.80 (m, 4H), 2.96 (t, J=5.9 Hz, 2H), 2.70-2.45 (m, 6H), 2.31 (s, 2H), 2.15-2.06 (m, 1H), 1.96 (s, 3H), 1.86 (d, J=16.4 Hz, 2H), 1.58 (s, 7H).

### Example 59. 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinic acid (Compound 124)

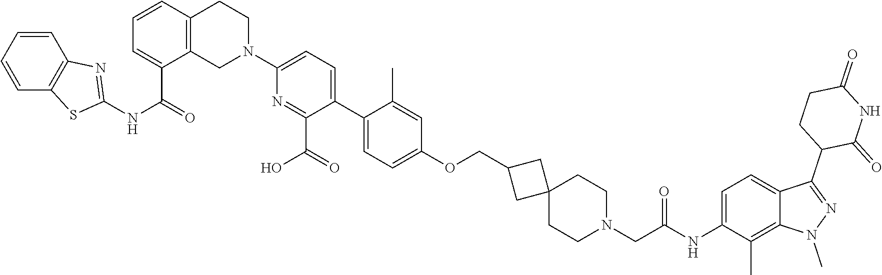 [SMILES: Cc1cc(OCC2CC3(CCN(CC(=O)Nc4ccc5c(C6CCC(=O)NC6=O)nn(C)c5c4C)CC3)C2)ccc1-c1ccc(N2CCc3cccc(C(=O)Nc4nc5ccccc5s4)c3C2)nc1C(=O)O]

### Step 1: 1,7-dimethyl-6-nitro-indazole

To a solution of 7-methyl-6-nitro-1H-indazole (24 g, 135.47 mmol, 1 equiv.) in DMF (200 mL) was added potassium carbonate (46.81 g, 338.68 mmol, 2.5 equiv.) and Mel (48.07 g, 338.68 mmol, 21.08 mL, 2.5 equiv.). The mixture was stirred at 40° C. for 2 hours. The reaction mixture was diluted with H₂O (800 mL) and extracted with ethyl acetate (210 mL). The combined organic layers were washed with brine (100 mL), dried over sodium sulfate, filtered, and concentrated under reduced pressure to give a residue, which was purified by column chromatography (SiO₂, petroleum ether/ethyl acetate=1/0 to 3/1) to afford 1,7-dimethyl-6-nitro-indazole (9.3 g) was obtained as a yellow solid.

LC-MS (ESI): m/z=192 [M+H]⁺

### Step 2: 1,7-dimethylindazol-6-amine

A solution of 1,7-dimethyl-6-nitro-indazole (9 g, 46.13 mmol, 1 equiv.) in MeOH (90 mL) was degassed and purged with N₂ three times. To this mixture, Pd on carbon (10 wt %, 4.91 g, 4.61 mmol, 0.1 equiv.) was added under N₂ atmosphere. The mixture was degassed and purged with H₂ three times. The mixture was stirred at 20° C. for 16 hours. The reaction mixture was filtered and concentrated under reduced pressure to give a residue, which was triturated with a solution (petroleum ether:ethyl acetate=1:1) for 3 min to afford 1,7-dimethylindazol-6-amine (7 g) was obtained as a brown solid.

LC-MS (ESI): m/z=162 [M+H]⁺

### Step 3: 6-bromo-1,7-dimethyl-indazole

To a solution of 1,7-dimethylindazol-6-amine (7 g, 42.99 mmol, 1 equiv.) in MeCN (70 mL) was added tert-butyl nitrite (8.87 g, 85.98 mmol, 10.23 mL, 2 equiv.). The mixture was stirred at 0° C. for 30 min. To this mixture, dibromocopper (14.40 g, 64.48 mmol, 3.02 mL, 1.5 equiv.) and CuBr (3.08 g, 21.49 mmol, 654.65 μL, 0.5 equiv.) was added. The mixture was stirred at 60° C. for 1 hour. The reaction mixture was added into ethyl acetate (100 mL) and washed with cold ammonia (60 mL). The mixture was concentrated under reduced pressure to give a residue, which was purified by column chromatography (SiO₂, petroleum ether/ethyl acetate=1/0 to 10/1) to afford 6-bromo-1,7-dimethyl-indazole (9 g) was obtained as a yellow solid.

LC-MS (ESI): m/z=225 [M+H]⁺

### Step 4: 6-bromo-3-iodo-1,7-dimethyl-indazole

A solution of 6-bromo-1,7-dimethyl-indazole (12.2 g, 46.61 mmol, 1 equiv.) in DMSO (100 mL) was added NIS (20.97 g, 93.23 mmol, 2 equiv.) The mixture was stirred at 90° C. for 3 hours. The reaction mixture was diluted with H₂O (200 mL) and extracted with ethyl acetate. The combined organic layers were washed with saturated sodium sulfite solution and concentrated under reduced pressure to give a residue, which was purified by column chromatography (SiO₂, petroleum ether/ethyl acetate=1/0 to 10/1) to afford 6-bromo-3-iodo-1,7-dimethyl-indazole (9.5 g) was obtained as a white solid.

LC-MS (ESI): m/z=352 [M+H]⁺

### Step 5: 6-bromo-3-(2,6-dibenzyloxy-3-pyridyl)-1,7-dimethyl-indazole

A solution of 6-bromo-3-iodo-1,7-dimethyl-indazole (6.6 g, 17.30 mmol, 1 equiv.) and 2,6-dibenzyloxy-3-(4,4,5,5-tetramethyl-1,3,2-dioxaborolan-2-yl)pyridine (8.66 g, 20.76 mmol, 1.2 equiv.) and cesium carbonate (16.91 g, 51.90 mmol, 3 equiv.) in THE (30 mL) and H₂O (3 mL) was degassed and purged with N₂ three times. To this mixture, Pd(dppf)Cl₂ (1.27 g, 1.73 mmol, 0.1 equiv.) was added under N₂ atmosphere. The mixture was degassed and purged with N₂ three times. The mixture was stirred at 60° C. for 16 hours. The reaction mixture was diluted with H₂O (60 mL) and extracted with ethyl acetate (120 mL). The combined organic layers were concentrated under reduced pressure to give a residue, which was purified by column chromatography (SiO₂, petroleum ether/ethyl acetate=1/0 to 5/1) to afford 6-bromo-3-(2,6-dibenzyloxy-3-pyridyl)-1,7-dimethyl-indazole (7.1 g) was obtained as a white solid.

LC-MS (ESI): m/z=514 [M+H]⁺

¹H NMR (400 MHz, DMSO-d₆) δ=7.86-7.84 (m, 1H), 7.53-7.44 (m, 2H), 7.44-7.24 (m, 9H), 7.18-7.16 (m, 1H), 6.60-6.58 (m, 1H), 5.43 (s, 4H), 4.31 (s, 3H), 2.82 (s, 3H)

### Step 6: tert-butyl (3-(2,6-bis(benzyloxy)pyridin-3-yl)-1,7-dimethyl-1H-indazol-6-yl)carbamate

A mixture of 6-bromo-3-(2,6-dibenzyloxy-3-pyridyl)-1,7-dimethyl-indazole (5.6 g, 10.89 mmol, 1 equiv.), NH₂Boc (2.55 g, 21.77 mmol, 2 equiv.), Pd(OAc)₂ (244.40 mg, 1.09 mmol, 0.1 equiv.), XPhos (1.04 g, 2.18 mmol, 0.2 equiv.) and cesium carbonate (10.64 g, 32.66 mmol, 3 equiv.) in dioxane (60 mL) was degassed and purged with N₂ three times. The mixture was stirred at 100° C. for 16 hours under N₂ atmosphere. The mixture was concentrated under reduced pressure to give a residue. The residue was purified by flash silica gel chromatography (0-20% ethyl acetate/petroleum ether) to give tert-butyl (3-(2,6-bis(benzyloxy)pyridin-3-yl)-1,7-dimethyl-1H-indazol-6-yl)carbamate (6.0 g, crude) as a yellow oil.

LC-MS (ESI): m/z=551.5 [M+H]⁺

¹H NMR (400 MHz, DMSO-d₆) δ=8.79 (s, 1H), 7.86 (d, J=8.0 Hz, 1H), 7.49-7.45 (m, 2H), 7.42-7.35 (m, 5H), 7.34-7.23 (m, 4H), 6.94 (d, J=9.2 Hz, 1H), 6.58 (d, J=8.0 Hz, 1H), 5.44 (d, J=11.6 Hz, 4H), 4.27 (s, 3H), 2.56 (s, 3H), 1.48 (s, 9H)

### Step 7: tert-butyl (3-(2,6-dioxopiperidin-3-yl)-1,7-dimethyl-1H-indazol-6-yl)carbamate

A mixture of tert-butyl (3-(2,6-bis(benzyloxy)pyridin-3-yl)-1,7-dimethyl-1H-indazol-6-yl)carbamate (6 g, 10.90 mmol, 1 equiv.), Pd on carbon (10 wt %, 2.90 g, 2.72 mmol, 0.25 equiv.) and Pd(OH)₂ on carbon (10 wt %, 3.83 g, 2.72 mmol, 0.25 equiv.) in EtOH (30 mL) and THE (30 mL) was degassed and purged with H₂ three times at 25° C. The mixture was stirred at 40° C. for 16 hours under H₂ atmosphere. The reaction mixture was filtered and concentrated under reduced pressure to give a filter cake. The filtrate concentrated under reduced pressure to give tert-butyl (3-(2,6-dioxopiperidin-3-yl)-1,7-dimethyl-1H-indazol-6-yl)carbamate (3.6 g) as a white solid.

LC-MS (ESI): m/z=373.2 [M+H]⁺

¹H NMR (400 MHz, DMSO-d₆) δ=10.87 (s, 1H), 8.79 (s, 1H), 7.41 (d, J=8.4 Hz, 1H), 6.99-6.90 (m, 1H), 4.30 (dd, J=5.2, 10.0 Hz, 1H), 4.20 (s, 3H), 2.69-2.58 (m, 2H), 2.52 (s, 3H), 2.37-2.14 (m, 2H), 1.46 (s, 9H)

### Step 8: 3-(6-amino-1,7-dimethyl-1H-indazol-3-yl)piperidine-2,6-dione

A solution of tert-butyl (3-(2,6-dioxopiperidin-3-yl)-1,7-dimethyl-1H-indazol-6-yl)carbamate (3.6 g, 9.67 mmol, 1 equiv.) in HCl/dioxane (36 mL) was degassed and purged with N₂ three times. The mixture was stirred at 25° C. for 2 hours under N₂ atmosphere. The reaction was concentrated under reduced pressure and the residue was purified by re-crystallization from petroleum ether (100 mL) at 25° C. to give 3-(6-amino-1,7-dimethyl-1H-indazol-3-yl)piperidine-2,6-dione (2.12 g) as a white solid

LC-MS (ESI): m/z=272.9 [M+H]⁺

¹H NMR (400 MHz, DMSO-d₆) δ=10.90 (s, 1H), 10.64-9.93 (m, 2H), 7.60 (d, J=8.4 Hz, 1H), 7.19 (d, J=8.8 Hz, 1H), 4.36 (dd, J=4.8, 10.2 Hz, 1H), 4.25 (s, 3H), 2.75-2.54 (m, 5H), 2.42-2.08 (m, 2H).

### Step 9: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinate

2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl]acetic acid (100 mg, 1 equiv., 127 mol) was suspended in DCM (5 mL). To the mixture was added HATU (53.1 mg, 1.1 equiv., 140 mol), followed by 3-(6-amino-1,7-dimethyl-1H-indazol-3-yl)piperidine-2,6-dione (41.5 mg, 1.2 equiv., 152 mol) and DIPEA (49.2 mg, 3 equiv., 381 mol). The mixture was heated overnight at 40° C. The mixture was then concentrated and extracted with ethyl acetate and water. The organic layer was separated, washed with brine, dried over sodium sulfate, filtered, and concentrated under reduced pressure to afford tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinate.

LC-MS (ESI): m/z=521.9 [M+2H]²⁺

### Step 10: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinic acid

tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinate (132 mg, 1 equiv., 127 mol) was dissolved in DCM (4 mL) and TFA (1 mL). The mixture was then stirred at room temperature overnight. The reaction mixture was concentrated under reduced pressure and purified by prep-HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1,7-dimethyl-1H-indazol-6-yl)amino)-2-oxoethyl)-7-azaspiro[3.5]nonan-2-yl)methoxy)-2-methylphenyl)picolinic acid (64 mg) as a yellow solid.

LC-MS (ESI): m/z=493.9 [M+2H]²⁺

¹H NMR (400 MHz, DMSO-d₆) δ 12.79 (s, 1H), 12.48 (s, 1H), 10.81 (s, 1H), 7.96 (d, J=7.9 Hz, 1H), 7.72 (d, J=8.0 Hz, 1H), 7.56 (d, J=7.4 Hz, 1H), 7.45-7.34 (m, 4H), 7.34-7.24 (m, 2H), 6.87 (dd, J=15.5, 8.6 Hz, 2H), 6.73 (d, J=2.6 Hz, 1H), 6.64 (dd, J=8.4, 2.6 Hz, 1H), 4.90 (s, 2H), 4.26 (dd, J=10.0, 5.1 Hz, 1H), 4.16 (s, 3H), 3.90-3.80 (m, 4H), 2.96 (t, J=6.0 Hz, 3H), 2.67-2.52 (m, 4H), 2.49 (s, 3H), 2.34-2.20 (m, 2H), 2.14-2.04 (m, 1H), 1.96 (s, 3H), 1.92-1.85 (m, 1H), 1.69 (s, 4H), 1.60 (s, 3H).

### Example 60. 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinic acid (Compound 128)

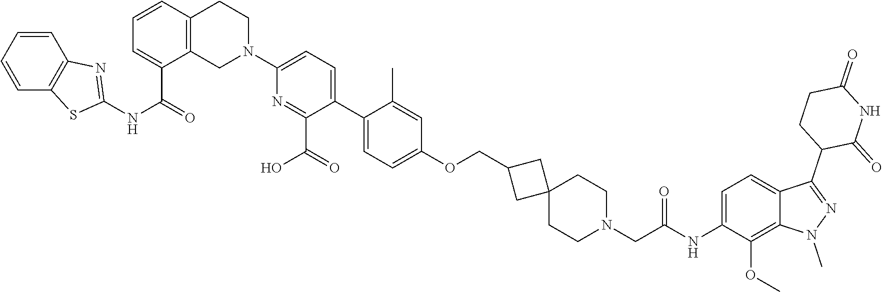 [SMILES: COc1c(NC(=O)CN2CCC3(CC2)CC(COc2ccc(-c4ccc(N5CCc6cccc(C(=O)Nc7nc8ccccc8s7)c6C5)nc4C(=O)O)c(C)c2)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinate

2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl]acetic acid (100 mg, 1 equiv., 127 mol) was suspended in pyridine (1 mL). To the mixture was added 3-(6-amino-7-methoxy-1-methyl-indazol-3-yl)piperidine-2,6-dione (54.9 mg, 1.5 equiv., 190 mol), followed by EDCI (29.2 mg, 1.2 equiv., 152 mol). The mixture was then stirred at room temperature for 4 hours. The mixture was extracted with ethyl acetate and water. The organic layer was separated, washed with brine, dried over sodium sulfate, filtered, and evaporated to dryness to give tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinate.

LC-MS (ESI): m/z=530.0 [M+2H]²⁺

### Step 2: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinic acid

tert-Butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinate (134 mg, 1 equiv., 127 mol) was dissolved in DCM (4 mL) and TFA (1 mL). The mixture was then stirred at room temperature overnight. The mixture was then concentrated and purified by HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-methylphenyl)picolinic acid (85 mg) as a white solid.

LC-MS (ESI): m/z=1002.5 [M+H]⁺

¹H NMR (400 MHz, DMSO-d₆) δ 10.82 (s, 1H), 9.78 (s, 1H), 7.95 (dd, J=9.9, 8.2 Hz, 2H), 7.72 (d, J=8.0 Hz, 1H), 7.55 (d, J=7.5 Hz, 1H), 7.39 (qd, J=7.9, 2.9 Hz, 4H), 7.34-7.24 (m, 2H), 6.86 (dd, J=14.1, 8.6 Hz, 2H), 6.73 (d, J=2.6 Hz, 1H), 6.64 (dd, J=8.4, 2.6 Hz, 1H), 4.90 (s, 2H), 4.27 (dd, J=9.9, 5.1 Hz, 1H), 4.07 (s, 3H), 3.90-3.84 (m, 3H), 3.82 (s, 4H), 3.06 (s, 2H), 2.96 (t, J=5.9 Hz, 2H), 2.65-2.45 (m, 6H), 2.10 (dd, J=13.4, 5.5 Hz, 1H), 1.96 (s, 3H), 1.89 (t, J=10.2 Hz, 2H), 1.66 (s, 2H), 1.57 (t, J=9.8 Hz, 4H).

### Example 61. 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinic acid (Compound

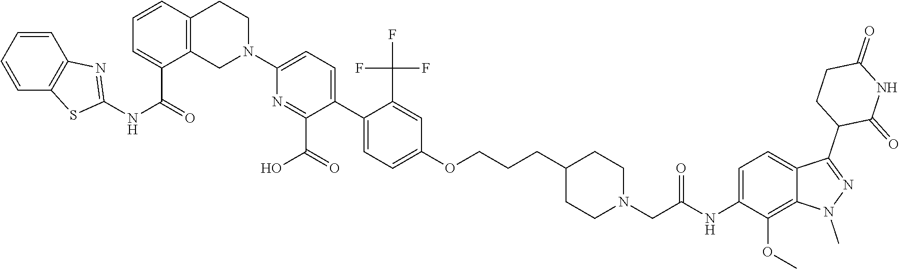 [SMILES: COc1c(NC(=O)CN2CCC(CCCOc3ccc(-c4ccc(N5CCc6cccc(C(=O)Nc7nc8ccccc8s7)c6C5)nc4C(=O)O)c(C(F)(F)F)c3)CC2)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinate

2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)piperidin-1-yl)acetic acid (100 mg, 1 equiv., 120 mol), 3-(6-amino-7-methoxy-1-methyl-indazol-3-yl)piperidine-2,6-dione (52.1 mg, 1.5 equiv., 181 mol), and EDCI hydrochloride (27.7 mg, 1.2 equiv., 145 mol) were dissolved in pyridine (5 mL). The mixture was stirred at room temperature for 2 hours. The mixture was then concentrated and extracted with ethyl acetate and water. The organic layer was separated, washed with brine, dried over sodium sulfate, filtered, and evaporated to dryness to afford tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinate.

LC-MS (ESI): m/z=1100.6 [M+H]⁺

### Step 2: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinic acid

tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinate (132 mg, 1 equiv., 120 mol) was dissolved in DCM (4 mL). To the mixture was added TFA (1 mL). The mixture was then stirred at room temperature overnight. The reaction mixture was concentrated, and then purified by prep-HPLC to afford 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-methoxy-1-methyl-1H-indazol-6-yl)amino)-2-oxoethyl)piperidin-4-yl)propoxy)-2-(trifluoromethyl)phenyl)picolinic acid (92 mg) as a white solid.

LC-MS (ESI): m/z=1044.5 [M+H]⁺

¹H NMR (400 MHz, DMSO-d₆) δ 12.78 (s, 1H), 12.38 (s, 1H), 10.82 (s, 1H), 9.79 (s, 1H), 7.96 (d, J=7.9 Hz, 1H), 7.72 (d, J=8.1 Hz, 1H), 7.56 (d, J=7.5 Hz, 1H), 7.47-7.23 (m, 7H), 7.13 (d, J=8.5 Hz, 1H), 6.92 (d, J=8.8 Hz, 1H), 6.61 (d, J=7.7 Hz, 1H), 4.96 (d, J=17.3 Hz, 1H), 4.89 (d, J=17.2 Hz, 1H), 4.27 (dd, J=10.0, 5.0 Hz, 1H), 4.06 (s, 3H), 4.03 (t, J=4.1 Hz, 2H), 3.87 (t, J=5.9 Hz, 2H), 3.81 (s, 3H), 3.09 (s, 2H), 2.96 (t, J=5.9 Hz, 2H), 2.88 (s, 2H), 2.65-2.45 (m, 2H), 2.29 (dd, J=11.7, 7.0 Hz, 1H), 2.18 (s, 2H), 2.09 (dq, J=13.2, 5.3 Hz, 1H), 1.76-1.67 (m, 4H), 1.37 (d, J=8.6 Hz, 2H), 1.25 (s, 3H).

### Example 62. 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid (Compound 143)

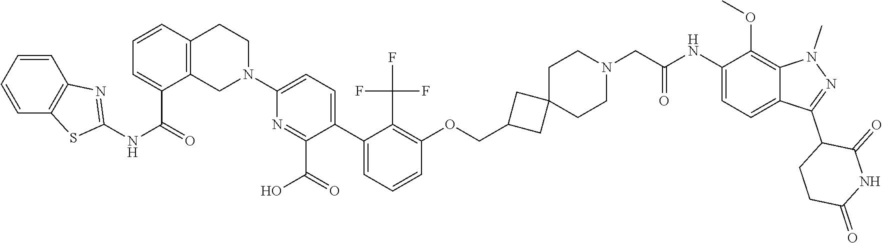 [SMILES: COc1c(NC(=O)CN2CCC3(CC2)CC(COc2cccc(-c4ccc(N5CCc6cccc(C(=O)Nc7nc8ccccc8s7)c6C5)nc4C(=O)O)c2C(F)(F)F)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate

2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)methyl)-7-azaspiro[3.5]nonan-7-yl)acetic acid (100 mg, 1 equiv., 119 mol), 3-(6-amino-7-methoxy-1-methyl-indazol-3-yl)piperidine-2,6-dione (51.4 mg, 1.5 equiv., 178 mol), and EDCI (27.3 mg, 1.2 equiv., 143 mol) were dissolved in pyridine (5 mL). The mixture was stirred at room temperature for 2 hours. The mixture was then concentrated and extracted with ethyl acetate and water. The organic layer was separated, washed with brine, dried over sodium sulfate, filtered, and evaporated to dryness to afford tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate.

LC-MS (ESI): m/z=1112.6 [M+H]⁺

### Step 2: 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid

tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate (132 mg, 1 equiv., 119 mol) was dissolved in DCM (4 mL). To the mixture was added TFA (1 mL). The mixture was then stirred at room temperature overnight. The reaction mixture was concentrated, and then purified by prep-HPLC to afford 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid (85 mg) as a white solid.

LC-MS (ESI): m/z=1056.6 [M+H]⁺

¹H NMR (400 MHz, DMSO-d₆) δ 12.78 (s, 1H), 12.39 (s, 1H), 10.82 (s, 1H), 9.78 (s, 1H), 7.96 (d, J=7.9 Hz, 1H), 7.72 (d, J=8.1 Hz, 1H), 7.55 (d, J=7.6 Hz, 1H), 7.43 (d, J=8.1 Hz, 1H), 7.42-7.31 (m, 4H), 7.35-7.23 (m, 2H), 7.13 (d, J=8.5 Hz, 1H), 6.92 (d, J=8.9 Hz, 1H), 6.61 (d, J=7.7 Hz, 1H), 5.00-4.85 (m, 2H), 4.27 (dd, J=9.9, 5.1 Hz, 1H), 4.07 (s, 3H), 4.00 (t, J=5.1 Hz, 2H), 3.87 (t, J=6.0 Hz, 2H), 3.82 (s, 3H), 3.05 (s, 2H), 2.96 (t, J=6.0 Hz, 2H), 2.61-2.45 (m, 1H), 2.32-2.20 (m, 1H), 2.09 (dd, J=13.2, 5.7 Hz, 1H), 1.84 (t, J=10.1 Hz, 2H), 1.69 (d, J=9.9 Hz, 4H), 1.56 (s, 2H).

### Example 63. 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid (Compound 144)

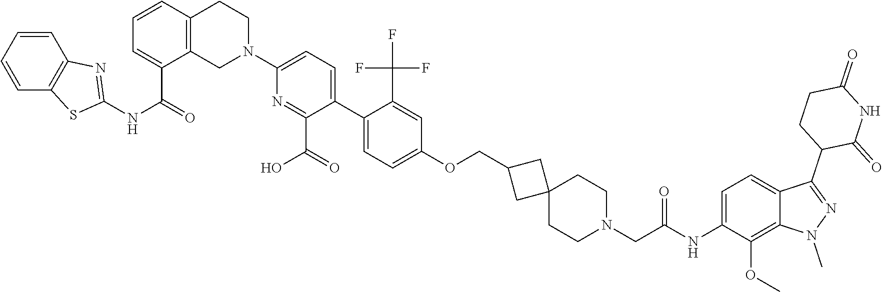 [SMILES: COc1c(NC(=O)CN2CCC3(CC2)CC(COc2ccc(-c4ccc(N5CCc6cccc(C(=O)Nc7nc8ccccc8s7)c6C5)nc4C(=O)O)c(C(F)(F)F)c2)C3)ccc2c(C3CCC(=O)NC3=O)nn(C)c12]

### Step 1: tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate

2-(2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)methyl)-7-azaspiro[3.5]nonan-7-yl)acetic acid (100 mg, 1 equiv., 119 mol), 3-(6-amino-7-methoxy-1-methyl-indazol-3-yl)piperidine-2,6-dione (51.4 mg, 1.5 equiv., 178 mol) and EDCI (27.3 mg, 1.2 equiv., 143 mol) were dissolved in pyridine (5 mL). The mixture was stirred at room temperature for −2 hours. The mixture was then concentrated and extracted with ethyl acetate and water. The organic layer was separated, washed with brine, dried over sodium sulfate, filtered, and evaporated to dryness to afford tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate.

LC-MS (ESI): m/z=1112.6 [M+H]⁺

### Step 2: 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid

tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinate (132 mg, 1 equiv., 119 mol) was dissolved in DCM (4 mL). To the mixture was added TFA (1 mL). The mixture was purged with N₂ and stirred at room temperature until complete. The solvents were removed, and the residue was purified by HPLC to afford 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-7-methoxy-1-methyl-1H-indazol-6-yl)amino)-2-oxoethyl)-7-azaspiro[3.5]nonan-2-yl)methoxy)-2-(trifluoromethyl)phenyl)picolinic acid (86 mg) as a white solid.

LC-MS (ESI): m/z=1056.5 [M+H]⁺

¹H NMR (400 MHz, DMSO-d₆) δ 10.82 (s, 1H), 9.78 (s, 1H), 7.95 (t, J=9.6 Hz, 2H), 7.72 (d, J=8.1 Hz, 1H), 7.56 (d, J=7.6 Hz, 1H), 7.38 (dd, J=8.9, 5.1 Hz, 4H), 7.29 (dt, J=12.0, 7.6 Hz, 2H), 7.17-7.05 (m, 3H), 6.92 (d, J=8.9 Hz, 1H), 4.97 (d, J=17.3 Hz, 1H), 4.89 (d, J=17.3 Hz, 1H), 4.27 (dd, J=9.9, 5.1 Hz, 1H), 4.07 (s, 3H), 3.99 (d, J=6.5 Hz, 2H), 3.87 (t, J=6.0 Hz, 2H), 3.82 (s, 3H), 3.07 (s, 1H), 2.96 (t, J=5.8 Hz, 2H), 2.61-2.45 (m, 4H), 2.26 (s, 1H), 2.10 (dd, J=13.4, 5.6 Hz, 1H), 1.90 (t, J=10.2 Hz, 2H), 1.67 (s, 2H), 1.65-1.56 (m, 4H).

### Example 64: 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinic acid (Compound 209a)

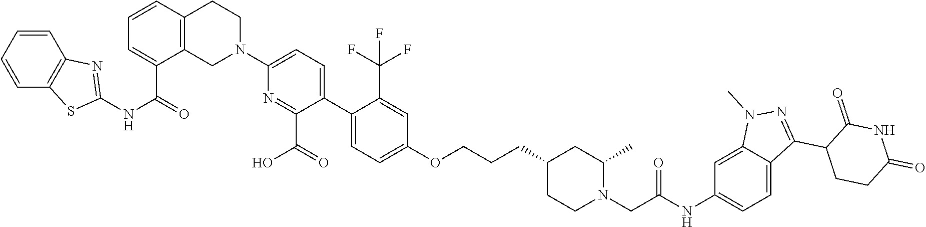 [SMILES: C[C@H]1C[C@@H](CCCOc2ccc(-c3ccc(N4CCc5cccc(C(=O)Nc6nc7ccccc7s6)c5C4)nc3C(=O)O)c(C(F)(F)F)c2)CCN1CC(=O)Nc1ccc2c(C3CCC(=O)NC3=O)nn(C)c2c1]

### Step 1: tert-butyl (2S)-4-(3-(4-bromo-3-(trifluoromethyl)phenoxy)propyl)-2-methylpiperidine-1-carboxylate

To a solution of tert-butyl (2S)-4-(3-hydroxypropyl)-2-methylpiperidine-1-carboxylate (1.4 g, 5.44 mmol, 1.0 equiv.) in toluene (20 mL) was added 2-(tributyl-phosphanylidene)acetonitrile (1.97 g, 8.16 mmol, 1.5 equiv.) and 4-bromo-3-(trifluoromethyl)phenol (1.57 g, 6.53 mmol, 1.2 equiv.). The mixture was stirred at 120° C. for 2 hours under microwave irradiation. The reaction mixture was concentrated under reduced pressure to give a residue, which was purified by reversed-phase HPLC to give tert-butyl (2S)-4-(3-(4-bromo-3-(trifluoromethyl)phenoxy)propyl)-2-methylpiperidine-1-carboxylate (2.0 g) as a yellow oil.

LC-MS (ESI): m/z=425.8 [M−56]*.

### Step 2: tert-butyl (2S,4S)-4-(3-(4-bromo-3-(trifluoromethyl)phenoxy)propyl)-2-methylpiperidine-1-carboxylate and tert-butyl (2S,4R)-4-(3-(4-bromo-3-(trifluoromethyl)phenoxy)propyl)-2-methylpiperidine-1-carboxylate

The isomers of tert-butyl (2S)-4-(3-(4-bromo-3-(trifluoromethyl)phenoxy)propyl)-2-methylpiperidine-1-carboxylate were further separated by SFC (column: DAICEL CHTRALPAK IG (250 mm×30 mm, 10 um); mobile phase: [CO₂-MeOH(0.1% NH₃·H₂O)]; B %:15%, isocratic elution mode) to give tert-butyl (2S,4S)-4-(3-(4-bromo-3-(trifluoromethyl)phenoxy)propyl)-2-methylpiperidine-1-carboxylate (300 mg) (Peak 1) and tert-butyl (2S,4R)-4-(3-(4-bromo-3-(trifluoromethyl)phenoxy)propyl)-2-methylpiperidine-1-carboxylate (1.4 g) (Peak 2) as yellow oils.

Peak 1:

¹H NMR (400 MHz, CDCl₃) δ=7.57 (d, J=8.8 Hz, 1H), 7.21 (d, J=2.8 Hz, 1H), 6.92-6.88 (m, 1H), 3.96 (t, J=6.4 Hz, 2H), 3.93-3.85 (m, 1H), 3.77-3.69 (m, 1H), 3.11-3.00 (m, 1H), 2.00-1.87 (m, 1H), 1.85-1.73 (m, 3H), 1.47 (s, 12H), 1.20-1.14 (m, 5H).

Peak 2:

¹H NMR (400 MHz, CDCl₃) δ=7.57 (d, J=8.8 Hz, 1H), 7.21 (d, J=2.8 Hz, 1H), 6.93-6.87 (m, 1H), 4.51-4.33 (m, 1H), 3.96 (t, J=6.4 Hz, 3H), 2.90-2.78 (m, 1H), 1.86-1.77 (m, 2H), 1.74-1.63 (m, 2H), 1.55 (s, 1H), 1.47 (s, 9H), 1.40-1.27 (m, 3H), 1.13 (d, J=6.8 Hz, 3H), 1.10-1.01 (m, 1H).

### Step 3: (2S,4S)-4-(3-(4-bromo-3-(trifluoromethyl)phenoxy)propyl)-2-methylpiperidine

To a solution of tert-butyl (2S,4S)-4-(3-(4-bromo-3-(trifluoromethyl)phenoxy)propyl)-2-methylpiperidine-1-carboxylate (300 mg, 624.53 mol, 1.0 equiv.) in DCM (2 mL) was added HCl/dioxane (2 M, 5 mL, 16.01 equiv.). The mixture was stirred at 25° C. for 1 hour. The reaction mixture was concentrated under reduced pressure to afford (2S,4S)-4-(3-(4-bromo-3-(trifluoromethyl)phenoxy)propyl)-2-methylpiperidine (250 mg) as a yellow solid.

LC-MS (ESI): m/z=381.9 [M+H]⁺

### Step 4: ethyl 2-((2S,4S)-4-(3-(4-bromo-3-(trifluoromethyl)phenoxy)propyl)-2-methylpiperidin-1-yl)acetate

To a solution of (2S,4S)-4-(3-(4-bromo-3-(trifluoromethyl)phenoxy)propyl)-2-methylpiperidine (250 mg, 599.95 mol, 1.0 equiv., HCl) in DMF (3 mL) was added potassium carbonate (248.75 mg, 1.80 mmol, 3.0 equiv.) and ethyl 2-bromoacetate (100.19 mg, 599.95 mol, 66.40 μL, 1.0 equiv.). The mixture was stirred at 40° C. for 1 hour. The reaction mixture was diluted with water (80 mL) and extracted with ethyl acetate (150 mL). The combined organic layers were washed with brine (100 mL), dried over sodium sulfate, filtered, and concentrated under reduced pressure to give ethyl 2-((2S,4S)-4-(3-(4-bromo-3-(trifluoromethyl)phenoxy)propyl)-2-methylpiperidin-1-yl)acetate (250 mg) as a yellow oil.

LC-MS (ESI): m/z=466.1 [M+H]⁺

### Step 5: tert-butyl 6-(8-(benzo[d]thiazol-2-ylcarbamoyl)-3,4-dihydroisoquinolin-2(1H)-yl)-3-(4-(3-((2S,4S)-1-(2-ethoxy-2-oxoethyl)-2-methylpiperidin-4-yl)propoxy)-2-(trifluoromethyl)phenyl)picolinate

To a solution of ethyl 2-((2S,4S)-4-(3-(4-bromo-3-(trifluoromethyl)phenoxy)propyl)-2-methylpiperidin-1-yl)acetate (250 mg, 536.10 mol, 1.0 equiv.) in dioxane (6 mL) and water (1 mL) was added tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (394.06 mg, 643.32 mol, 1.2 equiv.), KF (93.44 mg, 1.61 mmol, 3.0 equiv.), and cataCXium A Pd G3 (39.04 mg, 53.61 mol, 0.1 equiv.). The mixture was stirred at 80° C. for 12 hours under a N₂ atmosphere. The reaction mixture was concentrated under reduced pressure to give a residue, which was purified by flash silica gel chromatography (0~60% ethyl acetate/petroleum ether) to afford tert-butyl 6-(8-(benzo[d]thiazol-2-ylcarbamoyl)-3,4-dihydroisoquinolin-2(1H)-yl)-3-(4-(3-((2S,4S)-1-(2-ethoxy-2-oxoethyl)-2-methylpiperidin-4-yl)propoxy)-2-(trifluoromethyl)phenyl)picolinate (200 mg) as a yellow solid.

LC-MS (ESI): m/z=872.6 [M+H]⁺

### Step 6: 2-((2S,4S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)-2-methylpiperidin-1-yl)acetic acid

To a solution of tert-butyl 6-(8-(benzo[d]thiazol-2-ylcarbamoyl)-3,4-dihydroisoquinolin-2(1H)-yl)-3-(4-(3-((2S,4S)-1-(2-ethoxy-2-oxoethyl)-2-methylpiperidin-4-yl)propoxy)-2-(trifluoromethyl)phenyl)picolinate (200 mg, 229.36 mol, 1.0 equiv.) in THE (2 mL) and water (0.5 mL) was added LiOH·H₂O (28.87 mg, 688.07 mol, 3.0 equiv.). The mixture was stirred at 40° C. for 1 hour. The pH was adjusted to 5-6 with HCl (1 M). The resulting mixture was filtered and the filter cake concentrated under reduced pressure to give 2-((2S,4S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)-2-methylpiperidin-1-yl)acetic acid (180 mg) as a yellow solid.

LC-MS (ESI): m/z=844.6 [M+H]⁺

¹H NMR (400 MHz, DMSO-d₆) δ=7.79-7.68 (m, 2H), 7.51 (d, J=8.0 Hz, 1H), 7.42 (d, J=8.8 Hz, 1H), 7.30-7.18 (m, 5H), 7.16-7.02 (m, 2H), 6.93 (d, J=8.8 Hz, 1H), 5.16-5.06 (m, 2H), 4.05 (t, J=6.0 Hz, 2H), 3.90 (t, J=6.0 Hz, 2H), 3.21-3.18 (m, 1H), 3.12-3.08 (m, 1H), 2.99 (t, J=5.6 Hz, 2H), 2.72-2.54 (m, 3H), 1.77-1.59 (m, 4H), 1.46-1.18 (m, 5H), 1.06 (s, 12H)

### Step 7: tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinate

To a solution of 2-((2S,4S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-(trifluoromethyl)phenoxy)propyl)-2-methylpiperidin-1-yl)acetic acid (180 mg, 213.28 mol, 1.0 equiv.) in pyridine (4 mL) was added EDCI (61.33 mg, 319.92 mol, 1.5 equiv.) and 3-(6-amino-1-methyl-indazol-3-yl)piperidine-2,6-dione (66.10 mg, 255.94 mol, 1.2 equiv.). The mixture was stirred at 40° C. for 1 hour. The reaction mixture was concentrated under reduced pressure to give a residue. The reaction mixture was diluted with water (80 mL) and extracted with DCM (150 mL). The combined organic layers were washed with brine (100 mL), dried over sodium sulfate, filtered, and concentrated under reduced pressure to afford tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinate (180 mg) as a yellow solid.

LC-MS (ESI): m/z=1084.4 [M+H]⁺

### Step 8: 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinic acid

To a solution of tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinate (180 mg, 166.02 mol, 1.0 equiv.) in DCM (2 mL) was added TFA (4 mL). The mixture was stirred at 40° C. for 1 hour. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-(trifluoromethyl)phenyl)picolinic acid (90.6 mg) as an off-white solid.

LC-MS (ESI): m/z=1028.7 [M+H]⁺

¹H NMR (400 MHz, DMSO-d₆) δ=13.04-12.21 (m, 2H), 10.89 (s, 1H), 9.86 (s, 1H), 8.08-8.01 (m, 2H), 7.80 (d, J=8.0 Hz, 1H), 7.68-7.60 (m, 2H), 7.51-7.43 (m, 3H), 7.42-7.33 (m, 2H), 7.27-7.15 (m, 4H), 7.00 (d, J=8.8 Hz, 1H), 5.10-4.93 (m, 2H), 4.39-4.31 (m, 1H), 4.06 (t, J=6.0 Hz, 2H), 3.95 (s, 2H), 3.94 (s, 3H), 3.50-3.40 (m, 1H), 3.18-3.08 (m, 1H), 3.04 (t, J=6.0 Hz, 2H), 3.01-2.90 (m, 1H), 2.71-2.61 (m, 2H), 2.50-2.40 (m, 2H), 2.39-2.30 (m, 1H), 2.23-2.14 (m, 1H), 1.82-1.65 (m, 4H), 1.38-1.36 (m, 3H), 1.33-1.23 (m, 1H), 1.14-0.98 (m, 4H).

### Example 65: 6-(8-(benzo[d]thiazol-2-ylcarbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (Compound 219b)

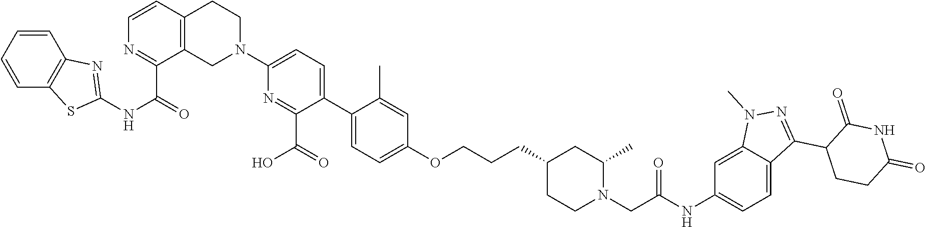 [SMILES: Cc1cc(OCCC[C@H]2CCN(CC(=O)Nc3ccc4c(C5CCC(=O)NC5=O)nn(C)c4c3)[C@@H](C)C2)ccc1-c1ccc(N2CCc3ccnc(C(=O)Nc4nc5ccccc5s4)c3C2)nc1C(=O)O]

### Step 1: 2-((2S,4S)-4-(3-(4-bromo-3-methylphenoxy)propyl)-2-methylpiperidin-1-yl)acetic acid

To a solution of ethyl 2-((2S,4S)-4-(3-(4-bromo-3-methylphenoxy)propyl)-2-methylpiperidin-1-yl)acetate (150 mg, 363.76 mol, 1.0 equiv.) in THE (4 mL) and water (1 mL) was added LiOH·H₂O (45.79 mg, 1.09 mmol, 3.0 equiv.). The mixture was stirred at 40° C. for 12 hours. The pH was adjusted to 5-6 with 1M HCl. The resulting mixture was filtered and the filter cake concentrated under reduced pressure to afford 2-((2S,4S)-4-(3-(4-bromo-3-methylphenoxy)propyl)-2-methylpiperidin-1-yl)acetic acid (140 mg, crude) as a yellow solid.

LC-MS (ESI): m/z=385.9 [M+H]⁺

### Step 2: 2-((2S,4S)-4-(3-(4-bromo-3-methylphenoxy)propyl)-2-methylpiperidin-1-yl)-N-(3-(2,6-dioxopiperidin-3-yl)-1-methyl-1H-indazol-6-yl)acetamide

To a solution of 2-((2S,4S)-4-(3-(4-bromo-3-methylphenoxy)propyl)-2-methylpiperidin-1-yl)acetic acid (140 mg, 364.29 mol, 1.0 equiv.) in pyridine (3 mL) was added EDCI (104.75 mg, 546.44 mol, 1.5 equiv.) and 3-(6-amino-1-methyl-indazol-3-yl)piperidine-2,6-dione (112.91 mg, 437.15 mol, 1.2 equiv.). The mixture was stirred at 40° C. for 1 hour. The reaction mixture was concentrated under reduced pressure to give a residue. The reaction mixture was diluted with water (80 mL) and extracted with DCM (150 mL, three times). The combined organic layers were washed with brine (100 mL), dried over sodium sulfate, filtered, and concentrated under reduced pressure to give a residue, which was purified by flash silica gel chromatography (0-100% ethyl acetate/petroleum ether) to afford 2-((2S,4S)-4-(3-(4-bromo-3-methylphenoxy)propyl)-2-methylpiperidin-1-yl)-N-(3-(2,6-dioxopiperidin-3-yl)-1-methyl-1H-indazol-6-yl)acetamide (140 mg) as a yellow solid.

LC-MS (ESI): m/z=626.1 [M+H]⁺

### Step 3: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

To a solution of 2-((2S,4S)-4-(3-(4-bromo-3-methylphenoxy)propyl)-2-methylpiperidin-1-yl)-N-(3-(2,6-dioxopiperidin-3-yl)-1-methyl-1H-indazol-6-yl)acetamide (140 mg, 224.07 mol, 1.0 equiv.) in dioxane (4 mL) and water (0.4 mL) was added tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (107.18 mg, 115.28 mol, 1.2 equiv.), KF (16.74 mg, 288.20 mol, 3.0 equiv.), and [2-(2-aminophenyl)phenyl]palladium(1+); bis(1-adamantyl)-butyl-phosphane; methanesulfonate (34.98 mg, 48.03 mol, 0.5 equiv.). The mixture was stirred at 100° C. for 2 hours. The reaction mixture was concentrated under reduced pressure to give a residue, which was purified by prep-TLC (DCM:MeOH=10:1) to afford tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (80 mg) as a yellow solid.

LC-MS (ESI): m/z=581.5 [M/2+H]⁺.

### Step 4: 6-(8-(benzo[d]thiazol-2-ylcarbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

To a solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (80 mg, 68.88 mol, 1.0 equiv.) in DCM (1 mL) was added TFA (2 mL). The mixture was stirred at 40° C. for 1 hour. The reaction mixture was concentrated under reduced pressure to give a residue, which was purified by prep-HPLC to give 6-(8-(benzo[d]thiazol-2-ylcarbamoyl)-3,4-dihydro-2,7-naphthyridin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (16.18 mg) as a yellow solid.

LC-MS (ESI): m/z=488.3 [M/2+H]⁺.

¹H NMR (400 MHz, DMSO-d₆) δ=12.83-12.46 (m, 1H), 12.31 (s, 1H), 10.89 (s, 1H), 10.00-9.67 (m, 1H), 8.57 (d, J=4.8 Hz, 1H), 8.08-8.01 (m, 2H), 7.82 (d, J=8.0 Hz, 1H), 7.65 (d, J=8.8 Hz, 1H), 7.58 (d, J=4.8 Hz, 1H), 7.51-7.46 (m, 2H), 7.40-7.34 (m, 1H), 7.24-7.19 (m, 1H), 7.05 (d, J=9.2 Hz, 1H), 6.94 (d, J=8.0 Hz, 1H), 6.81 (d, J=2.0 Hz, 1H), 6.75-6.70 (m, 1H), 5.24 (s, 2H), 4.36-4.29 (m, 1H), 3.99-3.92 (m, 7H), 3.25 (s, 1H), 3.08 (t, J=5.6 Hz, 2H), 3.01-2.87 (m, 1H), 2.68-2.51 (m, 4H), 2.40-2.28 (m, 2H), 2.21-2.14 (m, 1H), 2.05 (s, 3H), 1.79-1.64 (m, 4H), 1.41-1.23 (m, 4H), 1.17-0.96 (m, 4H).

### Example 66: 6-(8-(benzo[d]thiazol-2-ylcarbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (Compound 216d)

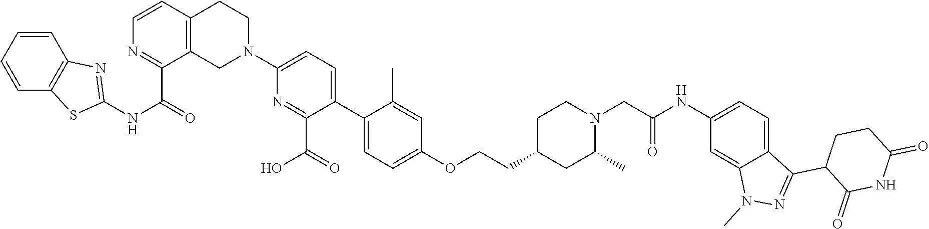 [SMILES: Cc1cc(OCC[C@@H]2CCN(CC(=O)Nc3ccc4c(C5CCC(=O)NC5=O)nn(C)c4c3)[C@H](C)C2)ccc1-c1ccc(N2CCc3ccnc(C(=O)Nc4nc5ccccc5s4)c3C2)nc1C(=O)O]

### Step 1: 2-((2R,4R)-4-(3-(3-bromo-2-methylphenoxy)propyl)-2-methylpiperidin-1-yl)acetic acid

To a solution of ethyl 2-((2R,4R)-4-(3-(3-bromo-2-methylphenoxy)propyl)-2-methylpiperidin-1-yl)acetate (200 mg, 485.01 mol, 1.0 equiv.) in THE (2 mL) and water (0.5 mL) was added LiOH·H₂O (61.06 mg, 1.46 mmol, 3.0 equiv.). The mixture was stirred at 40° C. for 4 hours. The reaction mixture was concentrated under reduced pressure to give a residue. The residue was diluted with water (30 mL) and the pH adjusted to pH=5-6 with 1N HCl. The mixture was extracted with DCM/MeOH (10/1, 150 mL). The combined organic layers were washed with brine (100 mL), dried over sodium sulfate, filtered, and concentrated under reduced pressure to give 2-((2R,4R)-4-(3-(3-bromo-2-methylphenoxy)propyl)-2-methylpiperidin-1-yl)acetic acid (180 mg) as a yellow oil.

LC-MS (ESI): m/z=386.0 [M+H]⁺

### Step 2: 2-((2R,4R)-4-(3-(3-bromo-2-methylphenoxy)propyl)-2-methylpiperidin-1-yl)-N-(3-(2,6-dioxopiperidin-3-yl)-1-methyl-1H-indazol-6-yl)acetamide

To a solution of 2-((2R,4R)-4-(3-(3-bromo-2-methylphenoxy)propyl)-2-methylpiperidin-1-yl)acetic acid (180 mg, 468.38 mol, 1.0 equiv.) and 3-(6-amino-1-methyl-indazol-3-yl)piperidine-2,6-dione (133.07 mg, 515.21 mol, 1.1 equiv.) in pyridine (3 mL) was added EDCI (134.68 mg, 702.56 mol, 1.5 equiv.). The mixture was stirred at 40° C. for 1 hour. The reaction mixture was concentrated under reduced pressure to give a residue. The residue was diluted with water (50 mL) and extracted with DCM (150 mL). The combined organic layers were washed with brine (100 mL), dried over sodium sulfate, filtered, and concentrated under reduced pressure to give a residue, which was purified by flash silica gel chromatography (60~100% ethyl acetate/petroleum ether) to afford 2-((2R,4R)-4-(3-(3-bromo-2-methylphenoxy)propyl)-2-methylpiperidin-1-yl)-N-(3-(2,6-dioxopiperidin-3-yl)-1-methyl-1H-indazol-6-yl)acetamide (220 mg) as a colorless oil.

LC-MS (ESI): m/z=626.0 [M+H]⁺

### Step 3: tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

A mixture of 2-((2R,4R)-4-(3-(3-bromo-2-methylphenoxy)propyl)-2-methylpiperidin-1-yl)-N-(3-(2,6-dioxopiperidin-3-yl)-1-methyl-1H-indazol-6-yl)acetamide (120 mg, 192.13 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (267.95 mg, 288.20 mol, 1.5 equiv.), Ad₂nBuP Pd G3 (27.98 mg, 38.43 mol, 0.2 equiv.) and KF (33.49 mg, 576.40 mol, 3.0 equiv.) in dioxane (4 mL) and water (0.4 mL) was degassed and purged with N₂ three times. The mixture was stirred at 100° C. for 4 hours under a N₂ atmosphere. The reaction mixture was concentrated under reduced pressure to give a residue, which was purified by flash silica gel chromatography (0-10% methanol/dichloromethane) to give tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (160 mg) as a yellow oil.

LC-MS (ESI): m/z=581.6 [M/2+H]⁺.

### Step 4: 6-(8-(benzo[d]thiazol-2-ylcarbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

To a solution of tert-butyl 6-(8-(benzo[d]thiazol-2-yl((2-(trimethylsilyl)ethoxy)methyl)carbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (160 mg, 137.75 mol, 1.0 equiv.) in DCM (2 mL) was added TFA (2 mL). The mixture was stirred at 25° C. for 16 hours. The reaction mixture was concentrated under reduced pressure to give a residue, which was purified by prep-HPLC to give 6-(8-(benzo[d]thiazol-2-ylcarbamoyl)-3,4-dihydro-2,7-naphthyridin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (31.64 mg) as a yellow solid.

LC-MS (ESI): m/z=488.4 [M/2+H]⁺.

¹H NMR (400 MHz, DMSO-d₆) δ=12.39-12.24 (m, 1H), 10.88 (s, 1H), 9.86-9.75 (m, 1H), 8.57 (d, J=5.2 Hz, 1H), 8.08-8.01 (m, 2H), 7.82 (d, J=8.0 Hz, 1H), 7.63 (d, J=8.8 Hz, 1H), 7.58 (d, J=4.8 Hz, 1H), 7.52-7.45 (m, 2H), 7.39-7.33 (m, 1H), 7.25-7.20 (m, 1H), 7.14-7.04 (m, 2H), 6.90 (d, J=8.4 Hz, 1H), 6.65 (d, J=7.2 Hz, 1H), 5.25 (s, 2H), 4.32 (dd, J=5.2, 10.0 Hz, 1H), 4.01-3.94 (m, 4H), 3.92 (s, 3H), 3.08 (t, J=6.0 Hz, 2H), 3.03-2.84 (m, 2H), 2.67-2.60 (m, 2H), 2.42-2.30 (m, 3H), 2.25-2.09 (m, 2H), 1.92 (s, 3H), 1.81-1.75 (m, 2H), 1.71-1.65 (m, 2H), 1.43-1.37 (m, 2H), 1.35-1.17 (m, 2H), 1.09-0.98 (m, 4H)

### Example 67: 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid [6-(8-(benzo[d]thiazol-2-ylcarbamoyl)-3,4-dihydroisoquinolin-2(1H)-yl)-3-(4-(((1S*,2R*)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid] (Compound 1003a)

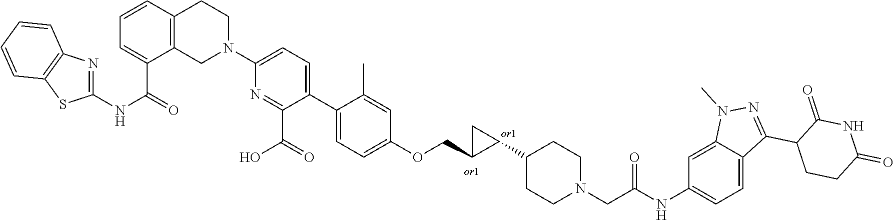 [SMILES: Cc1cc(OC[C@H]2C[C@@H]2C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: tert-butyl 4-((1R,2S)-2-((4-bromo-3-methylphenoxy)methyl)cyclopropyl)piperidine-1-carboxylate

A mixture of tert-butyl 4-((1R,2S)-2-(hydroxymethyl)cyclopropyl)piperidine-1-carboxylate (400 mg, 1.57 mmol, 1 equiv.), 4-bromo-3-methyl-phenol (351.58 mg, 1.88 mmol, 1.2 equiv.) and 2-(tributyl-phosphanylidene)acetonitrile (491.49 mg, 2.04 mmol, 1.3 equiv.) in toluene (10 mL) was degassed and purged with N₂ three times. The mixture was stirred at 130° C. for 2 hours under a N₂ atmosphere in a microwave reactor. The reaction mixture was concentrated under reduced pressure to give a residue, which was purified by reversed-phase HPLC to afford tert-butyl 4-((1R,2S)-2-((4-bromo-3-methylphenoxy)methyl)cyclopropyl)piperidine-1-carboxylate (400 mg) as a yellow oil.

¹H NMR (400 MHz, DMSO-d₆) δ=7.41 (d, J=8.8 Hz, 1H), 6.93 (d, J=2.8 Hz, 1H), 6.69 (dd, J=3.2, 8.8 Hz, 1H), 3.89 (d, J=10.4 Hz, 2H), 3.83-3.74 (m, 2H), 2.71-2.56 (m, 2H), 2.29 (s, 3H), 1.64 (t, J=8.0 Hz, 2H), 1.38 (s, 9H), 1.14-0.98 (m, 3H), 0.89-0.75 (m, 1H), 0.65-0.53 (m, 1H), 0.47-0.41 (m, 2H)

### Step 2: 4-((1R,2S)-2-((4-bromo-3-methylphenoxy)methyl)cyclopropyl)piperidine

A solution of tert-butyl 4-((1R,2S)-2-((4-bromo-3-methylphenoxy)methyl)cyclopropyl)piperidine-1-carboxylate (400 mg, 942.57 mol, 1 equiv.) in TFA (0.3 mL) and DCM (3 mL) was stirred at 25° C. for 2 hours. The reaction mixture was concentrated under reduced pressure to give 4-((1R,2S)-2-((4-bromo-3-methylphenoxy)methyl)cyclopropyl)piperidine (400 mg, crude) as a yellow oil.

### Step 3: ethyl 2-(4-((1R,2S)-2-((4-bromo-3-methylphenoxy)methyl)cyclopropyl)piperidin-1-yl)acetate

A mixture of 4-((1R,2S)-2-((4-bromo-3-methylphenoxy)methyl)cyclopropyl)piperidine (380 mg, 1.17 mmol, 1 equiv.), ethyl 2-bromoacetate (195.71 mg, 1.17 mmol, 129.70 μL, 1 equiv.) and TEA (592.93 mg, 5.86 mmol, 815.59 μL, 5 equiv.) in acetone (5 mL) was stirred at 25° C. for 1 hour under a N₂ atmosphere. The reaction mixture was concentrated under reduced pressure to give a residue, which was purified by flash silica gel chromatography (0-100% ethyl acetate/petroleum ether) to afford ethyl 2-(4-((1R,2S)-2-((4-bromo-3-methylphenoxy)methyl)cyclopropyl)piperidin-1-yl)acetate (400 mg) as a yellow solid.

LC-MS (ESI): m/z=411.7 [M+H]⁺

¹H NMR (400 MHz, DMSO-d₆) δ=7.41 (d, J=8.8 Hz, 1H), 6.93 (d, J=2.8 Hz, 1H), 6.69 (dd, J=3.2, 8.8 Hz, 1H), 4.06 (q, J=7.2 Hz, 2H), 3.79 (d, J=7.2 Hz, 2H), 3.15 (s, 2H), 2.79 (d, J=8.4 Hz, 2H), 2.28 (s, 3H), 2.07 (t, J=10.4 Hz, 2H), 1.69-1.56 (m, 2H), 1.31-1.23 (m, 2H), 1.18 (t, J=7.2 Hz, 3H), 0.98 (dd, J=4.8, 7.1 Hz, 1H), 0.67-0.53 (m, 2H), 0.47-0.37 (m, 2H)

### Step 4: tert-butyl 6-(8-(benzo[d]thiazol-2-ylcarbamoyl)-3,4-dihydroisoquinolin-2(1H)-yl)-3-(4-(((1S,2R)-2-(1-(2-ethoxy-2-oxoethyl)piperidin-4-yl)cyclopropyl)methoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (179.13 mg, 292.44 mol, 1.2 equiv.), ethyl 2-(4-((1R,2S)-2-((4-bromo-3-methylphenoxy)methyl)cyclopropyl)piperidin-1-yl)acetate (100 mg, 243.70 mol, 1 equiv.), cataCXium APd G3 (35.50 mg, 48.74 mol, 0.2 equiv.) and KF (1.5 M, 487.40 μL, 3 equiv.) in dioxane (5 mL) was degassed and purged with N₂ three times. The resulting mixture was stirred at 100° C. for 2 hours under a N₂ atmosphere. The reaction mixture was concentrated under reduced pressure to give a residue, which was purified by flash silica gel chromatography to give tert-butyl 6-(8-(benzo[d]thiazol-2-ylcarbamoyl)-3,4-dihydroisoquinolin-2(1H)-yl)-3-(4-(((1S,2R)-2-(1-(2-ethoxy-2-oxoethyl)piperidin-4-yl)cyclopropyl)methoxy)-2-methylphenyl)picolinate (170 mg) as a yellow solid.

LC-MS (ESI): m/z=816.3 [M+H]⁺

### Step 5: 2-(4-((1R,2S)-2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)methyl)cyclopropyl)piperidin-1-yl)acetic acid

A mixture of tert-butyl 6-(8-(benzo[d]thiazol-2-ylcarbamoyl)-3,4-dihydroisoquinolin-2(1H)-yl)-3-(4-(((1S,2R)-2-(1-(2-ethoxy-2-oxoethyl)piperidin-4-yl)cyclopropyl)methoxy)-2-methylphenyl)picolinate (170 mg, 208.33 mol, 1 equiv.) and LiOH·H₂O (43.71 mg, 1.04 mmol, 5 equiv.) in THF (2 mL) and water (1 mL) was stirred at 25° C. for 10 hours under a N₂ atmosphere. The reaction mixture was diluted with water (5 mL) and concentrated to form a turbid liquid. The pH of the turbid liquid was adjusted to pH=3 with 1 N HCl (5 mL) and the turbid liquid was extracted with DCM/MeOH (10:1, 15 mL). The combined organic layers were concentrated to afford 2-(4-((1R,2S)-2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)methyl)cyclopropyl)piperidin-1-yl)acetic acid (160 mg) as a yellow solid.

LC-MS (ESI): m/z=788.2 [M+H]⁺

### Step 6: tert-butyl 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinate

A mixture of 3-(6-amino-1-methyl-1H-indazol-3-yl)piperidine-2,6-dione (59.00 mg, 228.44 mol, 1.2 equiv.), 2-(4-((1R,2S)-2-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)methyl)cyclopropyl)piperidin-1-yl)acetic acid (150 mg, 190.36 mol, 1 equiv.) and EDCI (54.74 mg, 285.55 mol, 1.5 equiv.) in pyridine (3 mL) was stirred at 25° C. for 0.5 hour. The reaction mixture was concentrated under reduced pressure to give a residue. The residue was diluted with water (5 mL) and extracted with ethyl acetate (15 mL, three times). The combined organic phase was washed with water (15 mL, three times), dried over sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinate (150 mg) as a yellow solid.

LC-MS (ESI): m/z=514.8 [M/2+H]⁺.

### Step 7: 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid

### [6-(8-(benzo[d]thiazol-2-ylcarbamoyl)-3,4-dihydroisoquinolin-2(1H)-yl)-3-(4-(((1S*,2R*)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinate (150 mg, 145.88 mol, 1 equiv.) in DCM (1 mL) was added TFA (1 mL). The mixture was stirred at 25° C. for 16 hours. The reaction mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-(((1S,2R)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid [6-(8-(benzo[d]thiazol-2-ylcarbamoyl)-3,4-dihydroisoquinolin-2(1H)-yl)-3-(4-(((1S*,2R*)-2-(1-(2-((3-(2,6-dioxopiperidin-3-yl)-1-methyl-1H-indazol-6-yl)amino)-2-oxoethyl)piperidin-4-yl)cyclopropyl)methoxy)-2-methylphenyl)picolinic acid] (54.87 mg) as an off-white solid.

LC-MS (ESI): m/z=486.9 [M/2+H]⁺.

¹H NMR (400 MHz, DMSO-d₆) δ=13.06-12.58 (m, 1H), 10.88 (s, 1H), 9.89 (s, 1H), 8.14 (s, 1H), 8.07-7.99 (m, 2H), 7.79 (d, J=8.0 Hz, 1H), 7.66-7.60 (m, 2H), 7.49-7.41 (m, 3H), 7.39-7.32 (m, 2H), 7.21 (dd, J=1.6, 8.8 Hz, 1H), 6.91 (dd, J=8.4, 10.8 Hz, 2H), 6.78 (d, J=2.4 Hz, 1H), 6.69 (dd, J=2.4, 8.5 Hz, 1H), 4.96 (s, 2H), 4.32 (dd, J=5.2, 9.6 Hz, 1H), 3.93-3.88 (m, 5H), 3.82 (d, J=7.2 Hz, 2H), 3.16 (s, 2H), 3.02 (t, J=5.6 Hz, 2H), 2.92-2.86 (m, 2H), 2.67-2.59 (m, 2H), 2.35 (s, 1H), 2.20-2.11 (m, 3H), 2.02 (s, 3H), 1.74-1.63 (m, 2H), 1.48-1.34 (m, 2H), 1.08-0.98 (m, 1H), 0.75-0.59 (m, 2H), 0.51-0.40 (m, 2H)

### Example 68: 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinic acid (Compound 222)

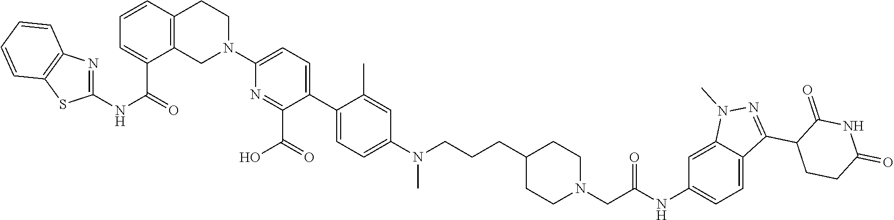 [SMILES: Cc1cc(N(C)CCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### Step 1: tert-butyl 4-(3-((4-bromo-3-methylphenyl)amino)propyl)piperidine-1-carboxylate

A mixture of 4-bromo-3-methylaniline (1.1 g, 5.91 mmol, 1.0 equiv.), tert-butyl 4-(3-bromopropyl)piperidine-1-carboxylate (1.99 g, 6.50 mmol, 1.1 equiv.), potassium carbonate (2.45 g, 17.74 mmol, 3.0 equiv.) and KI (981.48 mg, 5.91 mmol, 1.0 equiv.) in DMF (60 mL) was degassed and purged with N₂ three times. The mixture was stirred at 100° C. for 16 hours under a N₂ atmosphere. The reaction mixture was diluted with water (180 mL) and extracted with ethyl acetate (180 mL, three times). The combined organic layers were concentrated to give a residue, which was purified by flash silica gel chromatography (0-20% ethyl acetate/petroleum ether) to afford compound tert-butyl 4-(3-((4-bromo-3-methylphenyl)amino)propyl)piperidine-1-carboxylate (800 mg) as a red solid.

LC-MS (ESI): m/z=354.8 [M−56+H]⁺.

### Step 2: tert-butyl 4-(3-((4-bromo-3-methylphenyl)(methyl)amino)propyl)piperidine-1-carboxylate

A mixture of tert-butyl 4-(3-((4-bromo-3-methylphenyl)amino)propyl)piperidine-1-carboxylate (380 mg, 923.73 mol, 1.0 equiv.) and formaldehyde (277.36 mg, 9.24 mmol, 254.46 μL, 10.0 equiv.) in MeOH (5 mL) was stirred at 25° C. for 2.5 hours. NaBH₃CN (290.25 mg, 4.62 mmol, 5.0 equiv.) was then added and the resulting mixture was stirred at 25° C. for 0.5 hour. The mixture was concentrated under reduced pressure to give a residue, which was purified by flash silica gel chromatography (0-10% ethyl acetate/petroleum ether) to give tert-butyl 4-(3-((4-bromo-3-methylphenyl)(methyl)amino)propyl)piperidine-1-carboxylate (300 mg) as a yellow oil.

LC-MS (ESI): m/z=427.2 [M+H]⁺

### Step 3: 4-bromo-N,3-dimethyl-N-(3-(piperidin-4-yl)propyl)aniline

A solution of tert-butyl 4-(3-((4-bromo-3-methylphenyl)(methyl)amino)propyl)piperidine-1-carboxylate (300 mg, 705.22 mol, 1.0 equiv.) in HCl/dioxane (10 mL) was stirred at 25° C. for 1 hour. The mixture was concentrated under reduced pressure to afford 4-bromo-N,3-dimethyl-N-(3-(piperidin-4-yl)propyl)aniline (230 mg, crude) as a gray solid.

LC-MS (ESI): m/z=325.1 [M+H]⁺

### Step 4: ethyl 2-(4-(3-((4-bromo-3-methylphenyl)(methyl)amino)propyl)piperidin-1-yl)acetate

A mixture of 4-bromo-N,3-dimethyl-N-(3-(piperidin-4-yl)propyl)aniline (230 mg, 707.07 mol, 1.0 equiv.), ethyl 2-bromoacetate (129.89 mg, 777.78 mol, 86.08 μL, 1.1 equiv.) and TEA (357.74 mg, 3.54 mmol, 492.07 μL, 5.0 equiv.) in acetone (5 mL) was stirred at 25° C. for 1 hour. The mixture was concentrated under reduced pressure to give a residue, which was purified by flash silica gel chromatography (0-50% ethyl acetate/petroleum ether) to afford ethyl 2-(4-(3-((4-bromo-3-methylphenyl)(methyl)amino)propyl)piperidin-1-yl)acetate (250 mg) as a white solid.

LC-MS (ESI): m/z=413.2 [M+H]⁺

¹H NMR (400 MHz, DMSO-d₆) δ=7.25 (d, J=8.8 Hz, 1H), 6.62 (d, J=2.8 Hz, 1H), 6.43 (dd, J=2.4, 8.8 Hz, 1H), 4.06 (q, J=6.8 Hz, 2H), 3.24 (t, J=7.2 Hz, 2H), 3.13 (s, 2H), 2.83 (s, 3H), 2.77 (d, J=10.8 Hz, 2H), 2.25 (s, 3H), 2.08 (t, J=10.8 Hz, 2H), 1.57 (d, J=10.8 Hz, 2H), 1.49-1.41 (m, 2H), 1.22-1.07 (m, 8H)

### Step 5: tert-butyl 6-(8-(benzo[d]thiazol-2-ylcarbamoyl)-3,4-dihydroisoquinolin-2(1H)-yl)-3-(4-((3-(1-(2-ethoxy-2-oxoethyl)piperidin-4-yl)propyl)(methyl)amino)-2-methylphenyl)picolinate

A mixture of ethyl 2-(4-(3-((4-bromo-3-methylphenyl)(methyl)amino)propyl)piperidin-1-yl)acetate (150 mg, 364.63 mol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (268.02 mg, 437.56 mol, 1.2 equiv.), Ad₂nBuP Pd G3 (cataCXium® A Pd G3, 53.11 mg, 72.93 mol, 0.2 equiv.) and KF (63.55 mg, 1.09 mmol, 3.0 equiv.) in dioxane (5 mL) and water (0.5 mL) was degassed and purged with N₂ three times. The mixture was stirred at 100° C. for 1 hour under a N₂ atmosphere. The reaction mixture was quenched with water (3 mL), diluted with water (5 mL), and extracted with ethyl acetate (15 mL). The combined organic layers were concentrated to give a residue. The residue was purified by flash silica gel chromatography (0-70% ethyl acetate/petroleum ether) to give tert-butyl 6-(8-(benzo[d]thiazol-2-ylcarbamoyl)-3,4-dihydroisoquinolin-2(1H)-yl)-3-(4-((3-(1-(2-ethoxy-2-oxoethyl)piperidin-4-yl)propyl)(methyl)amino)-2-methylphenyl)picolinate (185 mg) as a yellow solid.

LC-MS (ESI): m/z=817.5 [M+H]⁺

### Step 6: 2-(4-(3-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenyl)(methyl)amino)propyl)piperidin-1-yl)acetic acid

A mixture of tert-butyl 6-(8-(benzo[d]thiazol-2-ylcarbamoyl)-3,4-dihydroisoquinolin-2(1H)-yl)-3-(4-((3-(1-(2-ethoxy-2-oxoethyl)piperidin-4-yl)propyl)(methyl)amino)-2-methylphenyl)picolinate (185 mg, 226.43 mol, 1.0 equiv.) and LiOH H₂O (28.50 mg, 679.28 mol, 3.0 equiv.) in THE (3 mL) and water (0.6 mL) was stirred at 25° C. for 2 hours. The reaction mixture was diluted with water (5 mL) and concentrated to form a turbid liquid. The pH of the turbid liquid was adjusted to 3 with 1 N HCl (5 mL) and the turbid liquid was extracted with DCM/MeOH (10:1, 15 mL). The combined organic layers were concentrated to give 2-(4-(3-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenyl)(methyl)amino)propyl)piperidin-1-yl)acetic acid (180 mg, crude) as a yellow solid.

LC-MS (ESI): m/z=789.5 [M+H]⁺

### Step 7: tert-butyl 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinate

A mixture of 2-(4-(3-((4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenyl)(methyl)amino)propyl)piperidin-1-yl)acetic acid (180 mg, 228.14 mol, 1.0 equiv.), 3-(6-amino-1-methyl-1H-indazol-3-yl)piperidine-2,6-dione (70.71 mg, 273.77 mol, 1.2 equiv.) and EDCI (65.60 mg, 342.21 mol, 1.5 equiv.) in pyridine (3 mL) was stirred at 25° C. for 1 hour. The reaction mixture was diluted with water (5 mL) and extracted with DCM/MeOH (10:1, 15 mL). The combined organic layers were concentrated to give tert-butyl 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinate (260 mg, crude) as a brown solid.

LC-MS (ESI): m/z=1029.7 [M+H]⁺

### Step 8: 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinic acid

A solution of tert-butyl 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinate (260 mg, 252.61 mol, 1.0 equiv.) in TFA (2 mL) and DCM (2 mL) was stirred at 40° C. for 1 hour. The mixture was concentrated under reduced pressure to give a residue, which was purified by prep-HPLC to give 6-(8-(benzo[d]thiazol-2-ylcarbamoyl)-3,4-dihydroisoquinolin-2(1H)-yl)-3-(4-((3-(1-(2-((3-(2,6-dioxopiperidin-3-yl)-1-methyl-1H-indazol-6-yl)amino)-2-oxoethyl)piperidin-4-yl)propyl)(methyl)amino)-2-methylphenyl)picolinic acid (145.32 mg) as a white solid.

LC-MS (ESI): m/z=973.6 [M+H]⁺

¹H NMR (400 MHz, DMSO-d₆) δ=12.99-12.72 (m, 1H), 12.71-12.06 (m, 1H), 10.89 (s, 1H), 10.72-9.24 (m, 1H), 8.13 (s, 1H), 8.06-8.00 (m, 2H), 7.79 (d, J=8.0 Hz, 1H), 7.63 (dd, J=8.4, 13.2 Hz, 2H), 7.49-7.41 (m, 3H), 7.36 (q, J=8.0 Hz, 2H), 7.18 (d, J=9.2 Hz, 1H), 6.93 (d, J=8.8 Hz, 1H), 6.82 (d, J=8.4 Hz, 1H), 6.52 (d, J=2.0 Hz, 1H), 6.50-6.44 (m, 1H), 4.96 (s, 2H), 4.33 (dd, J=5.2, 10.0 Hz, 1H), 3.92 (s, 3H), 3.91-3.88 (m, 2H), 3.28 (s, 3H), 3.02 (t, J=5.6 Hz, 3H), 2.87 (s, 3H), 2.64 (dt, J=5.6, 10.0 Hz, 3H), 2.45-2.29 (m, 2H), 2.28-2.05 (m, 2H), 2.02 (s, 3H), 1.72 (d, J=3.6 Hz, 2H), 1.57-1.49 (m, 2H), 1.49-1.12 (m, 6H).

### Example 69. 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinic acid (Compound 212)

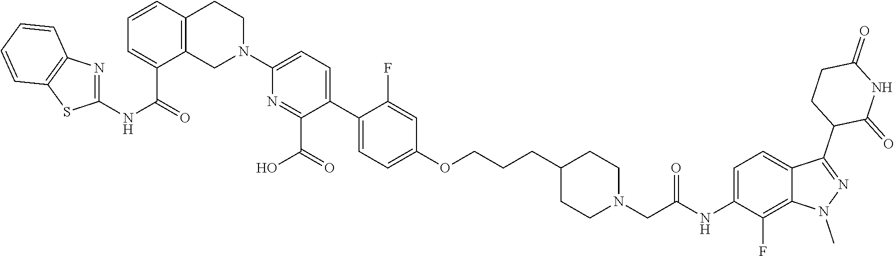 [SMILES: Cn1nc(C2CCC(=O)NC2=O)c2ccc(NC(=O)CN3CCC(CCCOc4ccc(-c5ccc(N6CCc7cccc(C(=O)Nc8nc9ccccc9s8)c7C6)nc5C(=O)O)c(F)c4)CC3)c(F)c21]

### Step 1: tert-butyl 4-(3-(4-bromo-3-fluorophenoxy)propyl)piperidine-1-carboxylate

To a solution of 4-bromo-3-fluorophenol (500 mg, 1 equiv., 2.62 mmol) and tert-butyl 4-(3-bromopropyl)piperidine-1-carboxylate (962 mg, 1.2 equiv., 3.14 mmol) in acetonitrile (10 mL) was added potassium carbonate (1.09 g, 3 equiv., 7.85 mmol). The mixture was stirred at 50° C. for 72 hours under a N₂ atmosphere. The reaction mixture was cooled to room temperature, filtered, and concentrated under reduced pressure to give tert-butyl 4-(3-(4-bromo-3-fluorophenoxy)propyl)piperidine-1-carboxylate (1.1 g) as a brown oil.

### Step 2: 4-(3-(4-bromo-3-fluorophenoxy)propyl)piperidine, HCi

A solution of tert-butyl 4-(3-(4-bromo-3-fluorophenoxy)propyl)piperidine-1-carboxylate (1.1 g, 1 equiv., 2.62 mmol) in HCl (95.5 mg, 655 μL, 1 equiv., 2.62 mmol) and 1,4-dioxane was stirred for 2 hours at room temperature. The reaction mixture was evaporated to dryness to afford 4-(3-(4-bromo-3-fluorophenoxy)propyl)piperidine, HCl (0.95 g) as a white solid.

LC-MS (ESI): m/z=316.1 [M+H]⁺

### Step 3: ethyl 2-(4-(3-(4-bromo-3-fluorophenoxy)propyl)piperidin-1-yl)acetate

To a solution of 4-(3-(4-bromo-3-fluorophenoxy)propyl)piperidine, HCl (924 mg, 1 equiv., 2.62 mmol) and ethyl bromoacetate (525 mg, 349 μL, 1.2 equiv., 3.14 mmol) in acetonitrile (10 mL) was added potassium carbonate (1.09 g, 3 equiv., 7.86 mmol). The mixture was stirred overnight at room temperature. The reaction mixture was filtered and concentrated to give ethyl 2-(4-(3-(4-bromo-3-fluorophenoxy)propyl)piperidin-1-yl)acetate (1.2 g) as a brown oil.

LC-MS (ESI): m/z=402.0 [M+H]⁺

### Step 4: tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-fluorophenyl)picolinate

To a solution of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (600 mg, 1 equiv., 980 mol) and ethyl 2-(4-(3-(4-bromo-3-fluorophenoxy)propyl)piperidin-1-yl)acetate (473 mg, 1.2 equiv., 1.18 mmol) in 1,4-dioxane (9 mL) and water (1 mL), Mesylate[(di(1-adamantyl)-n-butylphosphine)-2-(2′-amino-1,1′-biphenyl)]palladium(II) (71.3 mg, 0.1 equiv., 98.0 mol) was added, followed by potassium fluoride (171 mg, 3 equiv., 2.94 mmol). The mixture was stirred at 100° C. for 2 hours under a N₂ atmosphere. The reaction mixture was extracted with ethyl acetate and water. The organic layer was separated, washed with brine, dried over sodium sulfate, filtered, and concentrated under reduced pressure. The resulting residue was purified by flash silica gel chromatography (0-100% ethyl acetate/heptane) to give tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-fluorophenyl)picolinate (621 mg) as a brown oil.

LC-MS (ESI): m/z=808.5 [M+H]⁺

### Step 5: 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-fluorophenoxy)propyl)piperidin-1-yl)acetic acid

To a solution of tert-butyl 6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)propoxy)-2-fluorophenyl)picolinate (620 mg, 1 equiv., 767 mol) in THE (10 mL), LiGH (55.1 mg, 2.30 mL, 3 equiv., 2.30 mmol) was added. The mixture was stirred overnight at room temperature under a N₂ atmosphere. The mixture was extracted with ethyl acetate and water. The organic layer was dried over sodium sulfate, filtered, and concentrated under reduced pressure to afford 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-fluorophenoxy)propyl)piperidin-1-yl)acetic acid (427 mg) as a white solid.

LC-MS (ESI): m/z=780.4 [M+H]⁺

### Step 6: tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinate

To a solution of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-fluorophenoxy)propyl)piperidin-1-yl)acetic acid (80 mg, 1 equiv., 0.10 mmol) and 3-(6-amino-7-fluoro-1-methyl-1H-indazol-3-yl)piperidine-2,6-dione (43 mg, 1.5 equiv., 0.15 mmol) in pyridine (2 mL), EDC (24 mg, 1.2 equiv., 0.12 mmol) was added. The mixture was heated at 60° C. until completion. The mixture was purified by prep-HPLC to afford tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinate (15 mg) as a white solid.

LC-MS (ESI): m/z=982.5 [M+H]⁺

¹H NMR (400 MHz, DMSO-d₆) δ 10.84 (s, 1H), 9.64 (s, 1H), 7.95 (d, J=7.9 Hz, 1H), 7.71 (d, J=8.1 Hz, 1H), 7.53 (dd, J=17.8, 7.0 Hz, 2H), 7.47-7.38 (m, 3H), 7.38-7.33 (m, 2H), 7.28 (q, J=7.5 Hz, 3H), 7.17 (t, J=8.8 Hz, 2H), 6.86 (d, J=9.0 Hz, 1H), 6.77-6.66 (m, 3H), 4.92 (s, 2H), 4.30 (dd, J=10.4, 5.1 Hz, 1H), 4.04 (s, 3H), 3.92 (t, J=6.4 Hz, 2H), 3.85 (t, J=5.9 Hz, 2H), 3.08 (s, 3H), 2.94 (t, J=5.9 Hz, 2H), 2.84 (d, J=11.0 Hz, 2H), 2.56 (d, J=5.2 Hz, 2H), 2.33-2.25 (m, 2H), 2.11 (s, 4H), 1.66 (d, J=10.0 Hz, 5H), 1.31 (s, 2H), 1.22 (s, 4H).

### Step 7: 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinic acid

To a solution of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinate (15 mg, 14 mol, 1 equiv.) in DCM (3 mL) was added TFA (1 mL). The mixture was stirred overnight at room temperature under a N₂ atmosphere. The reaction mixture was concentrated under reduced pressure to give a residue, which was purified by prep-HPLC to afford 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-fluorophenyl)picolinic acid (10 mg) as a white solid.

LC-MS (ESI): m/z=982.5 [M+H]⁺

¹H NMR (400 MHz, DMSO-d₆) δ 10.84 (s, 1H), 9.64 (s, 1H), 7.95 (d, J=7.9 Hz, 1H), 7.71 (d, J=8.1 Hz, 1H), 7.53 (dd, J=17.8, 7.0 Hz, 2H), 7.47-7.38 (m, 3H), 7.38-7.33 (m, 2H), 7.28 (q, J=7.5 Hz, 3H), 7.17 (t, J=8.8 Hz, 2H), 6.86 (d, J=9.0 Hz, 1H), 6.77-6.66 (m, 3H), 4.92 (s, 2H), 4.30 (dd, J=10.4, 5.1 Hz, 1H), 4.04 (s, 3H), 3.92 (t, J=6.4 Hz, 2H), 3.85 (t, J=5.9 Hz, 2H), 3.08 (s, 3H), 2.94 (t, J=5.9 Hz, 2H), 2.84 (d, J=11.0 Hz, 2H), 2.56 (d, J=5.2 Hz, 2H), 2.33-2.25 (m, 2H), 2.11 (s, 4H), 1.66 (d, J=10.0 Hz, 5H), 1.31 (s, 2H), 1.22 (s, 4H).

### Example 82: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylic acid (Compound 207)

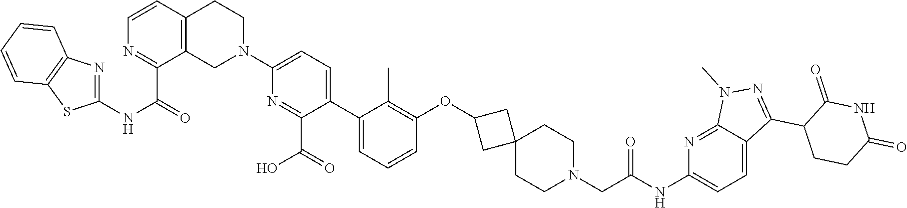 [SMILES: Cc1c(OC2CC3(CCN(CC(=O)Nc4ccc5c(C6CCC(=O)NC6=O)nn(C)c5n4)CC3)C2)cccc1-c1ccc(N2CCc3ccnc(C(=O)Nc4nc5ccccc5s4)c3C2)nc1C(=O)O]

### Step 1: tert-butyl (3-(2,6-bis(benzyloxy)pyridin-3-yl)-1-methyl-1H-pyrazolo[3,4-b]pyridin-6-yl)carbamate

A mixture of tert-butyl carbamate (1.44 g, 12.26 mmol, 2.0 equiv.) and 3-(2,6-bis(benzyloxy)pyridin-3-yl)-6-chloro-1-methyl-1H-pyrazolo[3,4-b]pyridine (2.8 g, 6.13 mmol, 1.0 equiv.) in dioxane (50 mL) was degassed and purged with N₂ three times. Pd(OAc)₂ (137.58 mg, 612.80 mol, 0.1 equiv.), cesium carbonate (5.99 g, 18.38 mmol, 3.0 equiv.), and XPhos (584.26 mg, 1.23 mmol, 0.2 equiv.) were then added. The mixture was stirred at 100° C. for 12 hours under a N₂ atmosphere. The reaction mixture was filtered and concentrated under reduced pressure to give a residue, which was purified by flash silica gel chromatography (0-8% ethyl acetate/petroleum ether) to give tert-butyl (3-(2,6-bis(benzyloxy)pyridin-3-yl)-1-methyl-1H-pyrazolo[3,4-b]pyridin-6-yl)carbamate (3.0 g) as a yellow oil.

LC-MS (ESI): m/z=538.3 [M+H]⁺

### Step 2: tert-butyl (3-(2,6-dioxopiperidin-3-yl)-1-methyl-1H-pyrazolo[3,4-b]pyridin-6-yl)carbamate

To a solution of tert-butyl (3-(2,6-bis(benzyloxy)pyridin-3-yl)-1-methyl-1H-pyrazolo[3,4-b]pyridin-6-yl)carbamate (3.0 g, 5.58 mmol, 1.0 equiv.) in EtOH (20 mL) and THE (20 mL) were added Pd on carbon (1.50 g, 1.41 mmol, 10% wt) and Pd(OH)₂ on carbon (1.50 g, 2.14 mmol, 20% wt) under a N₂ atmosphere. The suspension was degassed and purged with H₂ three times. The mixture was stirred under H₂ (15 psi) at 40° C. for 12 hours. The reaction mixture was filtered and concentrated under reduced pressure to give a residue, which was triturated with DCM at 25° C. for 30 minutes and filtered to give tert-butyl (3-(2,6-dioxopiperidin-3-yl)-1-methyl-1H-pyrazolo[3,4-b]pyridin-6-yl)carbamate (1.1 g) as a white solid.

LC-MS (ESI): m/z=360.1 [M+H]⁺

### Step 3: 3-(6-amino-1-methyl-1H-pyrazolo[3,4-b]pyridin-3-yl)piperidine-2,6-dione

A solution of tert-butyl (3-(2,6-dioxopiperidin-3-yl)-1-methyl-1H-pyrazolo[3,4-b]pyridin-6-yl)carbamate (1.1 g, 3.06 mmol, 1.0 equiv.) in dioxane (5 mL) was treated with HCl/dioxane (2 M, 25 mL). The mixture was stirred at 25° C. for 12 hours. The reaction mixture was filtered under reduced pressure to give 3-(6-amino-1-methyl-1H-pyrazolo[3,4-b]pyridin-3-yl)piperidine-2,6-dione (725.98 mg) as a white solid.

LC-MS (ESI): m/z=260.1 [M+H]⁺

¹H NMR (400 MHz, MeOD-d₄) δ=8.05 (d, J=9.2 Hz, 1H), 6.64 (d, J=9.2 Hz, 1H), 4.31 (dd, J=5.2, 10.4 Hz, 1H), 3.96 (s, 3H), 2.85-2.74 (d, J=5.6 Hz, 2H), 2.49-2.30 (m, 2H)

### Step 4: 2-[2-(3-bromo-2-methyl-phenoxy)-7-azaspiro[3.5]nonan-7-yl]-N-[3-(2,6-dioxo-3-piperidyl)-1-methyl-pyrazolo[3,4-b]pyridin-6-yl]acetamide

To a solution of 2-[2-(3-bromo-2-methyl-phenoxy)-7-azaspiro[3.5]nonan-7-yl]acetic acid (150 mg, 407.32 mol, 1.0 equiv.) and 3-(6-amino-1-methyl-1H-pyrazolo[3,4-b]pyridin-3-yl)piperidine-2,6-dione (144.54 mg, 488.78 mol, 1.2 equiv., HCl) in pyridine (3 mL) was added EDCI (117.12 mg, 610.97 mol, 1.5 equiv.). The mixture was stirred at 40° C. for 1 hour. The reaction mixture was concentrated under reduced pressure to give a residue, which was purified by flash silica gel chromatography (0-10% MeOH/DCM) to give 2-[2-(3-bromo-2-methyl-phenoxy)-7-azaspiro[3.5]nonan-7-yl]-N-[3-(2,6-dioxo-3-piperidyl)-1-methyl-pyrazolo[3,4-b]pyridin-6-yl]acetamide (140 mg) as a yellow solid.

LC-MS (ESI): m/z=610.9 [M+H]⁺

### Step 5: tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylate

A mixture of 2-[2-(3-bromo-2-methyl-phenoxy)-7-azaspiro[3.5]nonan-7-yl]-N-[3-(2,6-dioxo-3-piperidyl)-1-methyl-pyrazolo[3,4-b]pyridin-6-yl]acetamide (76.09 mg, 114.85 mol, 1.0 equiv.), tert-butyl 6-[8-[1,3-benzothiazol-2-yl(2-trimethylsilylethoxymethyl)carbamoyl]-3,4-dihydro-1H-2,7-naphthyridin-2-yl]-3-(4,4,5,5-tetramethyl-1,3,2-dioxaborolan-2-yl)pyridine-2-carboxylate (106.78 mg, 126.33 mol, 1.1 equiv.), KF (20.02 mg, 344.54 mol, 3.0 equiv.), and Ad₂nBuP Pd G3 (16.73 mg, 22.97 mol, 0.2 equiv.) in dioxane (2 mL) and water (0.2 mL) was degassed and purged with N₂ three times. The mixture was stirred at 100° C. for 2 hours in a microwave reactor. The reaction mixture was concentrated under reduced pressure to give a residue, which was purified by flash silica gel chromatography (0-70% ethyl acetate/petroleum ether) to give tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylate (70 mg) as a yellow solid.

LC-MS (ESI): m/z=1146.6 [M+H]⁺

### Step 6: 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylic acid

A solution of tert-butyl 6-[8-[1,3-benzothiazol-2-yl(2-trimethylsilylethoxymethyl)carbamoyl]-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylate (70 mg, 45.79 μmol, 1.0 equiv.) in DCM (1 mL) and TFA (2 mL) was stirred at 40° C. for 1 hour. The reaction mixture was concentrated under reduced pressure to give a residue, which was purified by prep-HPLC to give 6-[8-(1,3-benzothiazol-2-ylcarbamoyl)-3,4-dihydro-1H-2,7-naphthyridin-2-yl]-3-[3-[[7-[2-[[3-(2,6-dioxo-3-piperidyl)-1-methyl-pyrazolo[3,4-b]pyridin-6-yl]amino]-2-oxo-ethyl]-7-azaspiro[3.5]nonan-2-yl]oxy]-2-methyl-phenyl]pyridine-2-carboxylic acid (20.93 mg) as a yellow solid.

LC-MS (ESI): m/z=480.8 [M/2+H]⁺.

¹H NMR (400 MHz, DMSO-d₆) δ=12.33 (s, 1H), 11.39 (s, 1H), 10.95 (s, 1H), 9.73-9.65 (m, 1H), 8.57 (d, J=4.4 Hz, 1H), 8.26 (d, J=8.0 Hz, 1H), 8.05 (d, J=8.0 Hz, 1H), 7.96-7.85 (m, 1H), 7.82 (d, J=8.0 Hz, 1H), 7.59 (d, J=4.8 Hz, 1H), 7.51-7.46 (m, 2H), 7.39-7.34 (m, 1H), 7.12-7.06 (m, 2H), 6.73 (d, J=8.4 Hz, 1H), 6.66 (d, J=7.2 Hz, 1H), 5.25 (s, 2H), 4.75 (t, J=6.8 Hz, 1H), 4.46-4.36 (m, 1H), 4.28-4.18 (m, 2H), 4.00-3.96 (m, 5H), 3.20-3.13 (m, 2H), 3.09-3.06 (m, 2H), 2.71-2.59 (m, 4H), 2.46-2.37 (m, 3H), 2.22-2.15 (m, 1H), 1.98-1.89 (m, 7H), 1.87-1.80 (m, 2H). The following examples were synthesized using methods similar to those described in the examples above:

| Example | Compound | LC-MS | |
|---|---|---|---|
| No. | No. | (ESI) | ¹H NMR |
| 70 | 192 | m/z = 1013.3 | ¹H NMR (400 MHz, DMSO-d₆) δ = 12.97-12.77 (m, 1H), 12.61- |
| | | [M + H]⁺ | 12.35 (m, 1H), 10.92 (s, 1H), 10.26-9.90 (m, 1H), 8.29-8.18 (m, 1H), |
| | | | 8.07-7.92 (m, 2H), 7.79 (d, J = 8.0 Hz, 1H), 7.65-7.59 (m, 1H), 7.50- |
| | | | 7.32 (m, 6H), 7.00 (t, J = 9.6 Hz, 2H), 6.72-6.65 (m, 1H), 5.07-4.96 |
| | | | (m, 2H), 4.94-4.86 (m, 1H), 4.39-4.31 (m, 1H), 3.98-3.90 (m, 5H), |
| | | | 3.26-3.08 (m, 2H), 3.05-3.00 (m, 2H), 2.75-2.54 (m, 4H), 2.47- |
| | | | 2.31 (m, 5H), 2.24-2.14 (m, 1H), 1.95-1.60 (m, 6H). |
| 71 | 193 | m/z = 507.5 | ¹H NMR (400 MHz, DMSO-d₆) δ = 10.92 (s, 1H), 10.10 (s, 1H), 8.19 |
| | | [M/2 + H]⁺ | (t, J = 10.8 Hz, 1H), 8.01 (t, J = 8.8 Hz, 2H), 7.78 (d, J = 8.0 Hz, 1H), |
| | | | 7.62 (d, J = 7.2 Hz, 1H), 7.46-7.41 (m, 3H), 7.39-7.32 (m, 2H), 7.18- |
| | | | 7.14 (m, 1H), 7.13-7.07 (m, 2H), 6.96 (d, J = 8.8 Hz, 1H), 5.06-4.94 |
| | | | (m, 2H), 4.85 (t, J = 6.8 Hz, 1H), 4.34 (dd, J = 4.8, 10.0 Hz, 1H), 3.91- |
| | | | 3.90 (m, 5H), 3.20 (s, 2H), 3.03 (t, J = 5.2 Hz, 2H), 2.69-2.53 (m, 3H), |
| | | | 2.47-2.30 (m, 6H), 2.22-2.14 (m, 1H), 1.87-1.80 (m, 2H), 1.72- |
| | | | 1.61 (m, 4H). |
| 72 | 194 | m/z = 959.1 | ¹H NMR (400 MHz, DMSO-d₆) δ = 12.89-12.86 (m, 1H), 12.62- |
| | | [M + H]⁺ | 12.55 (m, 1H), 10.92 (s, 1H), 10.15-10.04 (m, 1H), 8.27-8.16 (m, |
| | | | 1H), 8.06-7.96 (m, 2H), 7.79 (d, J = 8.0 Hz, 1H), 7.62 (d, J = 7.2 Hz, |
| | | | 1H), 7.48-7.43 (m, 3H), 7.39-7.33 (m, 2H), 7.07 (t, J = 8.0 Hz, 1H), |
| | | | 6.97 (d, J = 8.8 Hz, 1H), 6.71 (d, J = 8.0 Hz, 1H), 6.63 (d, J = 7.6 Hz, |
| | | | 1H), 4.98 (s, 2H), 4.73 (t, J = 6.4 Hz, 1H), 4.37-4.31 (m, 1H), 3.96- |
| | | | 3.89 (m, 5H), 3.19 (s, 1H), 3.03 (t, J = 5.6 Hz, 2H), 2.69-2.60 (m, 2H), |
| | | | 2.42 (s, 6H), 2.19 (dd, J = 5.2, 8.0 Hz, 1H), 2.06-1.99 (m, 1H), 1.89 (s, |
| | | | 3H), 1.86-1.78 (m, 3H), 1.75-1.58 (m, 4H). |
| 73 | 195 | m/z = 959.3 | ¹H NMR (400 MHz, DMSO-d₆) δ = 12.87 (s, 1H), 12.61-12.56 (m, |
| | | [M + H]⁺ | 1H), 10.93 (s, 1H), 9.71-9.63 (m, 1H), 8.25 (d, J = 8.4 Hz, 1H), 8.04- |
| | | | 7.94 (m, 2H), 7.79 (d, J = 8.0 Hz, 1H), 7.62 (d, J = 7.6 Hz, 1H), 7.47- |
| | | | 7.43 (m, 3H), 7.39-7.33 (m, 2H), 6.93 (dd, J = 8.8, 14.4 Hz, 2H), 6.71 |
| | | | (d, J = 2.0 Hz, 1H), 6.63 (dd, J = 2.0, 8.4 Hz, 1H), 4.97 (s, 2H), 4.77- |
| | | | 4.72 (m, 1H), 4.36 (dd, J = 5.2, 10.8 Hz, 1H), 3.96 (s, 3H), 3.91 (t, J = |
| | | | 5.6 Hz, 2H), 3.54-3.39 (m, 2H), 3.22-3.05 (m, 2H), 3.02 (t, J = 5.6 |
| | | | Hz, 2H), 2.78-2.52 (m, 5H), 2.45-2.29 (m, 2H), 2.23-2.14 (m, 1H), |
| | | | 2.03 (s, 3H), 1.98-1.77 (m, 6H). |
| 74 | 188 | m/z = 515.9 | ¹H NMR (400 MHz, DMSO-d₆) δ = 12.86-12.44 (m, 1H), 10.91 (s, |
| | | [M/2 + H]⁺ | 1H), 9.73 (s, 1H), 8.16 (s, 1H), 8.03 (d, J = 7.6 Hz, 1H), 7.78 (d, J = 8.0 |
| | | | Hz, 1H), 7.64-7.58 (m, 2H), 7.51-7.43 (m, 4H), 7.42-7.32 (m, 3H), |
| | | | 6.99 (dd, J = 8.8, 16.4 Hz, 2H), 6.68 (d, J = 8.0 Hz, 1H), 5.07-4.86 (m, |
| | | | 3H), 4.37 (dd, J = 5.2, 10.3 Hz, 1H), 4.11 (s, 3H), 3.96-3.89 (m, 2H), |
| | | | 3.14 (s, 2H), 3.02 (t, J = 5.6 Hz, 2H), 2.75-2.54 (m, 4H), 2.46-2.31 |
| | | | (m, 5H), 2.21-2.14 (m, 1H), 1.86-1.78 (m, 2H), 1.74-1.62 (m, 4H). |
| 75 | 189 | m/z = 1030.3 | ¹H NMR (400 MHz, DMSO-d₆) δ = 13.25-12.16 (m, 2H), 10.91 (s, |
| | | [M + H]⁺ | 1H), 9.73 (s, 1H), 8.03 (d, J = 7.6 Hz, 1H), 7.79 (d, J = 8.0 Hz, 1H), |
| | | | 7.65-7.57 (m, 2H), 7.52-7.43 (m, 4H), 7.42-7.31 (m, 2H), 7.18- |
| | | | 7.06 (m, 3H), 6.98 (d, J = 8.8 Hz, 1H), 5.08-4.93 (m, 2H), 4.90-4.82 |
| | | | (m, 1H), 4.37 (dd, J = 5.2, 10.4 Hz, 1H), 4.11 (s, 3H), 3.94 (t, J = 5.6 |
| | | | Hz, 2H), 3.15 (s, 2H), 3.03 (t, J = 5.6 Hz, 2H), 2.71-2.61 (m, 2H), 2.59 |
| | | | (s, 2H), 2.47-2.41 (m, 4H), 2.37-2.29 (m, 1H), 2.22-2.13 (m, 1H), |
| | | | 1.84 (dd, J = 6.0, 12.4 Hz, 2H), 1.70 (d, J = 19.2 Hz, 4H) |
| 76 | 190 | m/z = 488.8 | ¹H NMR (400 MHz, DMSO-d₆) δ = 13.12-12.13 (m, 2H), 10.99- |
| | | [2M + H]⁺ | 10.82 (m, 1H), 10.03-9.52 (m, 1H), 8.13 (s, 1H), 8.03 (d, J = 7.6 Hz, |
| | | | 1H), 7.79 (d, J = 8.0 Hz, 1H), 7.68-7.52 (m, 2H), 7.51-7.43 (m, 4H), |
| | | | 7.39-7.33 (m, 2H), 7.07 (t, J = 8.0 Hz, 1H), 6.97 (d, J = 8.8 Hz, 1H), |
| | | | 6.72 (d, J = 8.0 Hz, 1H), 6.63 (d, J = 7.6 Hz, 1H), 4.98 (s, 2H), 4.77- |
| | | | 4.69 (m, 1H), 4.38 (dd, J = 5.2, 10.4 Hz, 1H), 4.11 (s, 3H), 3.92 (t, J = |
| | | | 4.8 Hz, 2H), 3.27-3.06 (m, 2H), 3.03 (t, J = 6.0 Hz, 2H), 2.77-2.52 |
| | | | (m, 5H), 2.48-2.29 (m, 4H), 2.21-2.14 (m, 1H), 1.89 (s, 3H), 1.87- |
| | | | 1.63 (m, 6H). |
| 77 | 191 | m/z = 976.4 | ¹H NMR (400 MHz, DMSO-d₆) δ = 13.12-12.40 (m, 1H), 11.07- |
| | | [M + H]⁺ | 10.78 (m, 1H), 9.84-9.61 (m, 1H), 8.15-8.14 (m, 1H), 8.03 (d, J = 7.8 |
| | | | Hz, 1H), 7.79 (d, J = 8.0 Hz, 1H), 7.66-7.56 (m, 2H), 7.52-7.41 (m, |
| | | | 4H), 7.36 (q, J = 7.6 Hz, 2H), 6.93 (dd, J = 8.8, 12.0 Hz, 2H), 6.70 (d, J = |
| | | | 1.6 Hz, 1H), 6.62 (d, J = 8.8 Hz, 1H), 4.97 (s, 2H), 4.76-4.68 (m, |
| | | | 1H), 4.41-4.34 (m, 1H), 4.11 (s, 3H), 3.95-3.86 (m, 2H), 3.14 (s, 2H), |
| | | | 3.05-3.00 (m, 2H), 2.73-2.54 (m, 4H), 2.45-2.31 (m, 5H), 2.21- |
| | | | 2.12 (m, 1H), 2.02 (s, 3H), 1.84-1.76 (m, 2H), 1.74-1.62 (m, 4H). |
| 78 | 156 | m/z = 947.2 | ¹H NMR (400 MHz, DMSO-d₆) δ = 10.86 (s, 1H), 8.00 (d, J = 7.2 Hz, |
| | | [M + H]⁺ | 1H), 7.78 (d, J = 8.0 Hz, 1H), 7.62 (d, J = 7.2 Hz, 1H), 7.54 (d, J = 8.8 |
| | | | Hz, 1H), 7.48-7.40 (m, 3H), 7.35 (td, J = 7.6, 11.2 Hz, 2H), 7.09-7.04 |
| | | | (m, 2H), 6.93 (d, J = 8.8 Hz, 1H), 6.86 (d, J = 8.4 Hz, 1H), 6.72 (dd, J = |
| | | | 2.0, 8.8 Hz, 1H), 6.63 (d, J = 7.6 Hz, 1H), 4.97 (s, 2H), 4.29 (dd, J = |
| | | | 5.2, 9.6 Hz, 1H), 4.16 (t, J = 6.0 Hz, 2H), 3.95 (s, 7H), 3.02 (s, 2H), |
| | | | 2.97 (s, 2H), 2.75 (t, J = 5.6 Hz, 2H), 2.64-2.59 (m, 2H), 2.31 (dd, J = |
| | | | 4.4, 9.6 Hz, 1H), 2.16 (d, J = 8.0 Hz, 1H), 2.02 (s, 2H), 1.89 (s, 3H), |
| | | | 1.77-1.72 (m, 2H), 1.66 (d, J = 12.4 Hz, 2H), 1.39-1.34 (m, 2H), 1.28 |
| | | | (td, J = 3.6, 7.2 Hz, 1H), 1.19-1.11 (m, 2H). |
| 79 | 204 | m/z = 489.3 | ¹H NMR (400 MHz, DMSO-d₆) δ = 12.83-12.49 (m, 1H), 12.41- |
| | | [M/2 + H]⁺ | 12.16 (m, 1H), 10.92 (s, 1H), 9.87-9.59 (m, 1H), 8.57 (d, J = 4.8 Hz, |
| | | | 1H), 8.13 (s, 1H), 8.05 (d, J = 7.6 Hz, 1H), 7.82 (d, J = 8.0 Hz, 1H), |
| | | | 7.64-7.56 (m, 2H), 7.52-7.46 (m, 3H), 7.39-7.33 (m, 1H), 7.12- |
| | | | 7.04 (m, 2H), 6.73 (d, J = 8.4 Hz, 1H), 6.65 (d, J = 7.6 Hz, 1H), 5.25 (s, |
| | | | 2H), 4.78-4.70 (m, 1H), 4.38 (dd, J = 4.8, 10.4 Hz, 1H), 4.11 (s, 3H), |
| | | | 4.01-3.95 (m, 2H), 3.15 (d, J = 4.4 Hz, 2H), 3.08 (t, J = 5.2 Hz, 2H), |
| | | | 2.75-2.57 (m, 4H), 2.47-2.32 (m, 5H), 2.21-2.14 (m, 1H), 1.91 (s, |
| | | | 3H), 1.84 (dd, J = 6.0, 7.6 Hz, 2H), 1.71 (d, J = 15.6 Hz, 4H). |
| 80 | 202 | m/z = 507.9 | ¹H NMR (400 MHz, DMSO-d₆) δ = 12.70-12.41 (m, 1H), 12.34- |
| | | [M/2 + H]⁺ | 12.27 (m, 1H), 10.91 (s, 1H), 10.12-10.07 (m, 1H), 8.57 (d, J = 10.0 |
| | | | Hz, 1H), 8.20 (d, J = 9.2 Hz, 1H), 8.05 (d, J = 8.0 Hz, 1H), 8.02-7.98 |
| | | | (m, 1H), 7.82 (d, J = 8.4 Hz, 1H), 7.58 (d, J = 4.8 Hz, 1H), 7.47 (d, J = |
| | | | 8.8 Hz, 3H), 7.39-7.34 (m, 1H), 7.08 (d, J = 8.4 Hz, 1H), 7.03 (d, J = |
| | | | 8.4 Hz, 1H), 6.69 (d, J = 8.0 Hz, 1H), 5.31-5.19 (m, 2H), 4.93-4.86 |
| | | | (m, 1H), 4.38-4.30 (m, 1H), 4.04-3.97 (m, 2H), 3.94 (s, 3H), 3.22- |
| | | | 3.18(m, 2H), 3.10-3.06 (m, 2H), 2.64-2.57 (m, 4H), 2.46-2.41 (m, |
| | | | 4H), 2.23-2.13 (m, 2H), 1.86-1.80 (m, 2H), 1.71-1.63 (m, 4H). |
| 81 | 206 | m/z = 507.8 | ¹H NMR (400 MHz, DMSO-d₆) δ = 12.67-12.45 (m, 1H), 12.43- |
| | | [M/2 + H]⁺ | 12.16 (m, 1H), 10.92 (s, 1H), 10.14-10.04 (m, 1H), 8.57 (d, J = 4.8 Hz, |
| | | | 1H), 8.20 (d, J = 8.8 Hz, 1H), 8.05 (d, J = 7.6 Hz, 1H), 8.00 (d, J = 8.8 |
| | | | Hz, 1H), 7.82 (d, J = 8.2 Hz, 1H), 7.59 (d, J = 4.8 Hz, 1H), 7.53-7.45 |
| | | | (m, 2H), 7.36 (t, J = 7.2 Hz, 1H), 7.19-7.06 (m, 4H), 5.34-5.20 (m, |
| | | | 2H), 4.94-4.78 (m, 1H), 4.40-4.28 (m, 1H), 4.05-3.97 (m, 2H), 3.94 |
| | | | (s, 3H), 3.20 (s, 4H), 3.10-3.05 (m, 2H), 2.67-2.61 (m, 2H), 2.44- |
| | | | 2.32 (m, 4H), 2.32-2.07 (m, 2H), 1.87-1.81 (m, 2H), 1.73-1.64 (m, |
| | | | 4H) |
| 83 | 203 | m/z = 1031.3 | ¹H NMR (400 MHz, DMSO-d₆) δ = 12.56-12.25 (m, 1H), 10.92 (s, |
| | | [M + H]⁺ | 1H), 9.74 (s, 1H), 8.57 (d, J = 4.8 Hz, 1H), 8.05 (d, J = 8.0 Hz, 1H), |
| | | | 7.82 (d, J = 8.0 Hz, 1H), 7.59 (d, J = 5.6 Hz, 2H), 7.52-7.45 (m, 3H), |
| | | | 7.39-7.33 (m, 1H), 7.21-7.16 (m, 1H), 7.14-7.05 (m, 3H), 5.26 (d, J = |
| | | | 13.6 Hz, 2H), 4.87 (t, J = 6.4 Hz, 1H), 4.37 (dd, J = 4.8, 10.0 Hz, 1H), |
| | | | 4.11 (s, 3H), 4.03-3.95 (m, 2H), 3.15 (s, 2H), 3.10-3.06 (m, 2H), 2.72- |
| | | | 2.59 (m, 3H), 2.47-2.43 (m, 4H), 2.40-2.30 (m, 2H), 2.20-2.14 (m, |
| | | | 1H), 1.89-1.81 (m, 2H), 1.78-1.65 (m, 4H). |
| 84 | 205 | m/z = 489.3 | ¹H NMR (400 MHz, DMSO-d₆) δ = 13.31-11.65 (m, 2H), 10.92 (s, |
| | | [M/2 + H]⁺ | 1H), 9.82-9.65 (m, 1H), 8.57 (d, J = 4.8 Hz, 1H), 8.05 (d, J = 8.0 Hz, |
| | | | 1H), 7.82 (d, J = 8.0 Hz, 1H), 7.63-7.56 (m, 2H), 7.52-7.45 (m, 3H), |
| | | | 7.41-7.33 (m, 1H), 7.04 (d, J = 8.8 Hz, 1H), 6.93 (d, J = 8.4 Hz, 1H), |
| | | | 6.72 (d, J = 2.0 Hz, 1H), 6.64 (dd, J = 2.0, 8.4 Hz, 1H), 5.24 (s, 2H), |
| | | | 4.78-4.69 (m, 1H), 4.37 (dd, J = 4.8, 10.0 Hz, 1H), 4.11 (s, 3H), 3.98- |
| | | | 3.95 (m, 2H), 3.16-3.14 (m, 2H), 3.10-3.05 (m, 2H), 2.75-2.54 (m, |
| | | | 4H), 2.47-2.32 (m, 5H), 2.20-2.14 (m, 1H), 2.04 (s, 3H), 1.84-1.76 |
| | | | (m, 2H), 1.74-1.65 (m, 4H). |
| 85 | 208a | m/z = 1004.5 | ¹H NMR (400 MHz, DMSO-d₆) δ = 12.86-12.46 (m, 2H), 10.89 (s, |
| | | [M + H]⁺ | 1H), 10.03 (s, 1H), 8.04 (dd, J = 8.8, 13.2 Hz, 2H), 7.79 (d, J = 8.0 Hz, |
| | | | 1H), 7.62 (d, J = 7.2 Hz, 1H), 7.49-7.44 (m, 4H), 7.39-7.36 (m, 2H), |
| | | | 7.12-7.08 (t, J = 8.0 Hz, 1H), 6.97 (d, J = 8.8 Hz, 1H), 6.89 (d, J = 8.4 |
| | | | Hz, 1H), 6.63 (d, J = 7.6 Hz, 1H), 4.98 (s, 2H), 4.33 (dd, J = 5.2, 10.0 |
| | | | Hz, 1H), 4.12 (s, 3H), 3.98 (t, J = 6.0 Hz, 2H), 3.92 (d, J = 5.6 Hz, 2H), |
| | | | 3.89 (s, 3H), 3.10 (d, J = 17.2 Hz, 2H), 3.03 (t, J = 6.0 Hz, 2H), 2.70- |
| | | | 2.62 (m, 3H), 2.35-2.33 (m, 1H), 2.18-2.12 (m, 1H), 1.91 (s, 3H), |
| | | | 1.80-1.75 (m, 4H), 1.68-1.61 (m, 2H), 1.54-1.42 (m, 4H), 1.34- |
| | | | 1.30 (m, 1H), 1.05 (d, J = 6.4 Hz, 3H). |
| 86 | 208b | m/z = 1004.2 | ¹H NMR (400 MHz, DMSO-d₆) δ = 12.92-12.79 (m, 1H), 12.65- |
| | | [M + H]⁺ | 12.48 (m, 1H), 10.89 (s, 1H), 10.00 (br s, 1H), 8.03 (d, J = 7.6 Hz, 2H), |
| | | | 7.79 (d, J = 8.0 Hz, 1H), 7.62 (d, J = 7.6 Hz, 1H), 7.47-7.42 (m, 4H), |
| | | | 7.39-7.33 (m, 2H), 7.13-7.05 (t, J = 8.0 Hz, 1H), 6.97 (d, J = 8.8 Hz, |
| | | | 1H), 6.89 (d, J = 8.0 Hz, 1H), 6.63 (d, J = 7.2 Hz, 1H), 4.98 (s, 2H), |
| | | | 4.33 (dd, J = 5.2, 10.0 Hz, 1H), 4.13 (s, 3H), 3.97 (t, J = 6.4 Hz, 2H), |
| | | | 3.94-3.89 (m, 5H), 3.03 (t, J = 5.6 Hz, 2H), 3.00-2.87 (m, 2H), 2.70- |
| | | | 2.59 (m, 2H), 2.44-2.26 (m, 4H), 2.16 (dd, J = 5.2, 13.6 Hz, 1H), 1.91 |
| | | | (s, 3H), 1.83-1.74 (m, 4H), 1.49-1.39 (m, 3H), 1.34-1.14 (m, 2H), |
| | | | 1.07 (d, J = 4.4 Hz, 3H) |
| 87 | 210a | m/z = 1004.4 | ¹H NMR (400 MHz, DMSO-d₆) δ = 12.90-12.79 (m, 1H), 12.67- |
| | | [M + H]⁺ | 12.43 (m, 1H), 10.89 (s, 1H), 10.05-10.00 (m, 1H), 8.03 (d, J = 7.6 Hz, |
| | | | 1H), 7.79 (d, J = 8.0 Hz, 1H), 7.62 (d, J = 6.8 Hz, 1H), 7.51-7.42 (m, |
| | | | 5H), 7.36 (d, J = 9.2 Hz, 2H), 6.97-6.91 (m, 2H), 6.79 (s, 1H), 6.72 |
| | | | (dd, J = 1.6, 8.4 Hz, 1H), 4.97 (s, 2H), 4.36-4.31 (m, 1H), 4.14 (s, 3H), |
| | | | 4.14-3.96 (m, 2H), 3.93-3.87 (m, 5H), 3.14-3.07 (m, 2H), 3.02 (t, J = |
| | | | 5.6 Hz, 2H), 2.65-2.60 (m, 2H), 2.38-2.34 (m, 1H), 2.24-2.12 (m, |
| | | | 2H), 2.03 (s, 3H), 1.79-1.71 (m, 4H), 1.68-1.57 (m, 2H), 1.55-1.49 |
| | | | (m, 1H), 1.45-1.25 (m, 5H), 1.09-1.04 (m, 2H) |
| 88 | 210b | m/z = 1004.4 | ¹H NMR (400 MHz, DMSO-d₆) δ = 12.86 (s, 1H), 12.72-12.47 (m, |
| | | [M + H]⁺ | 1H), 10.90 (s, 1H), 10.01 (s, 1H), 8.03 (d, J = 8.0 Hz, 2H), 7.79 (d, J = |
| | | | 8.0 Hz, 1H), 7.62 (d, J = 8.0 Hz, 1H), 7.49-7.42 (m, 4H), 7.40-7.32 |
| | | | (m, 2H), 6.99-6.89 (m, 2H), 6.79 (d, J = 2.0 Hz, 1H), 6.74-6.69 (m, |
| | | | 1H), 4.97 (s, 2H), 4.37-4.31 (m, 1H), 4.14 (s, 3H), 3.95 (d, J = 6.0 Hz, |
| | | | 2H), 3.93-3.91 (m, 5H), 3.53-3.39 (m, 1H), 3.02 (t, J = 6.0 Hz, 2H), |
| | | | 2.98-2.92 (m 1H), 2.75-2.52 (m, 3H), 2.48-2.28 (m, 3H), 2.21-2.11 |
| | | | (m, 1H), 2.03 (s, 3H), 1.75 (s, 4H), 1.49-1.21 (m, 5H), 1.07 (s, 3H). |
| 89 | 209b | m/z = 1028.3 | ¹H NMR (400 MHz, DMSO-d₆) δ = 10.88 (s, 1H), 9.81 (s, 1H), 8.07- |
| | | [M + H]⁺ | 8.00 (m, 2H), 7.79 (d, J = 7.6 Hz, 1H), 7.67-7.60 (m, 2H), 7.49-7.42 |
| | | | (m, 3H), 7.37 (dd, J = 7.2, 12.8 Hz, 2H), 7.19 (d, J = 10.0 Hz, 4H), 7.02- |
| | | | 6.91 (m, 1H), 5.00 (d, J = 14.0 Hz, 2H), 4.33 (dd, J = 5.2, 9.8 Hz, 1H), |
| | | | 4.07 (t, J = 6.4 Hz, 2H), 3.97-3.91 (m, 5H), 3.25 (s, 2H), 3.04 (d, J = |
| | | | 5.2 Hz, 2H), 2.72-2.58 (m, 5H), 2.37-2.29 (m, 2H), 2.21-2.14 (m, |
| | | | 1H), 1.82-1.73 (m, 2H), 1.72-1.61 (m, 2H), 1.58-1.51 (m, 2H), 1.45- |
| | | | 1.38 (m, 2H), 1.03 (d, J = 6.6 Hz, 3H) |
| 90 | 208c | m/z = 1004.4 | ¹H NMR (400 MHz, DMSO-d₆) δ = 12.89-12.83 (m, 1H), 12.65- |
| | | [M + H]⁺ | 12.55 (m, 1H), 10.90 (s, 1H), 10.10-9.93 (m, 1H), 8.04 (d, J = 8.0 Hz, |
| | | | 2H), 7.80 (d, J = 8.0 Hz, 1H), 7.63 (d, J = 7.6 Hz, 1H), 7.49-7.45 (m, |
| | | | 4H), 7.40-7.33 (m, 2H), 7.11 (t, J = 8.0 Hz, 1H), 6.98 (d, J = 8.8 Hz, |
| | | | 1H), 6.90 (d, J = 8.2 Hz, 1H), 6.64 (d, J = 7.6 Hz, 1H), 4.99 (s, 2H), |
| | | | 4.34 (dd, J = 4.8, 10.0 Hz, 1H), 4.13 (s, 3H), 3.99 (t, J = 6.4 Hz, 2H), |
| | | | 3.94-3.91 (m, 2H), 3.90 (s, 3H), 3.24 (s, 1H), 3.13-3.09 (m, 2H), 3.04 |
| | | | (t, J = 5.6 Hz, 2H), 2.71-2.57 (m, 4H), 2.38-2.31 (m, 1H), 2.19-2.13 |
| | | | (m, 1H), 1.92 (s, 3H), 1.83-1.73 (m, 3H), 1.72-1.60 (m, 2H), 1.58- |
| | | | 1.49 (m, 1H), 1.49-1.41 (m, 2H), 1.38-1.26 (m, 1H), 1.06 (d, J = 6.4 |
| | | | Hz, 3H) |
| 91 | 208d | m/z = 1004.3 | ¹H NMR (400 MHz, DMSO-d₆) δ = 12.87 (s, 1H), 12.68-12.45 (m, |
| | | [M + H]⁺ | 1H), 10.89 (s, 1H), 10.00 (s, 1H), 8.08-7.99 (m, 2H), 7.79 (d, J = 7.2 |
| | | | Hz, 1H), 7.64-7.59 (m, 1H), 7.48-7.42 (m, 4H), 7.39-7.32 (m, 2H), |
| | | | 7.13-7.07 (m, 1H), 6.97 (d, J = 8.8 Hz, 1H), 6.88 (d, J = 8.0 Hz, 1H), |
| | | | 6.67-6.60 (m, 1H), 4.98 (s, 2H), 4.37-4.31 (m, 1H), 4.13 (s, 3H), 4.05- |
| | | | 3.94 (m, 2H), 3.94-3.85 (m, 5H), 3.05-3.01 (m, 2H), 2.99-2.92 (m, |
| | | | 2H), 2.65-2.54 (m, 2H), 2.43-2.32 (m, 4H), 2.19-2.12 (m, 1H), 1.90 |
| | | | (s, 3H), 1.84-1.74 (m, 4H), 1.46-1.41 (m, 2H), 1.29-1.20 (m, 3H), |
| | | | 1.06 (d, J = 6.0 Hz, 3H). |
| 92 | 210c | m/z = 503.1 | ¹H NMR (400 MHz, DMSO-d₆) δ = 13.00-12.77 (m, 1H), 12.69- |
| | | [M/2 + H]⁺ | 12.40 (m, 1H), 10.90 (s, 1H), 10.32-9.95 (m, 1H), 8.03 (d, J = 8.0 Hz, |
| | | | 1H), 7.79 (d, J = 8.0 Hz, 1H), 7.62 (d, J = 7.6 Hz, 1H), 7.53-7.28 (m, |
| | | | 7H), 6.94 (dd, J = 8.4, 13.6 Hz, 2H), 6.80 (d, J = 2.0 Hz, 1H), 6.72 (dd, |
| | | | J = 2.4, 8.4 Hz, 1H), 4.97 (s, 2H), 4.38-4.30 (m, 1H), 4.14 (s, 3H), 4.00- |
| | | | 3.85 (m, 8H), 3.16-3.07 (m, 1H), 3.02 (t, J = 5.2 Hz, 2H), 2.79-2.53 |
| | | | (m, 5H), 2.38-2.28 (m, 1H), 2.23-2.11 (m, 1H), 2.03 (s, 3H), 1.87- |
| | | | 1.59 (m, 6H), 1.46-1.27 (m, 4H), 1.17-0.99 (m, 2H). |
| 93 | 210d | m/z = 1004.3 | ¹H NMR (400 MHz, DMSO-d₆) δ = 13.03-12.30 (m, 2H), 10.90 (s, |
| | | [M + H]⁺ | 1H), 10.01 (s, 1H), 8.13-7.98 (m, 2H), 7.79 (d, J = 8.0 Hz, 1H), 7.62 |
| | | | (d, J = 7.6 Hz, 1H), 7.50-7.41 (m, 4H), 7.40-7.32 (m, 2H), 6.94 (dd, J = |
| | | | 8.8, 14.0 Hz, 2H), 6.80 (d, J = 2.4 Hz, 1H), 6.71 (dd, J = 2.4, 8.8 Hz, |
| | | | 1H), 4.97 (s, 2H), 4.34 (dd, J = 4.8, 9.6 Hz, 1H), 4.14 (s, 3H), 3.98- |
| | | | 3.88 (m, 7H), 3.51-3.41 (m, 2H), 3.06-2.93 (m, 4H), 2.68-2.59 (m, |
| | | | 2H), 2.43-2.36 (m, 2H), 2.20-2.14 (m, 1H), 2.03 (s, 3H), 1.81-1.71 |
| | | | (m, 4H), 1.50-1.37 (m, 3H), 1.37-1.14 (m, 2H), 1.07 (d, J = 6.0 Hz, |
| | | | 3H) |
| 94 | 209d | m/z = 515.0 | ¹H NMR (400 MHz, DMSO-d₆) δ = 13.20-11.99 (m, 2H), 10.89 (s, |
| | | [M/2 + H]⁺ | 1H), 8.08-7.97 (m, 2H), 7.79 (d, J = 8.0 Hz, 1H), 7.65 (dd, J = 8.4, |
| | | | 19.2 Hz, 2H), 7.51-7.41 (m, 3H), 7.36 (td, J = 7.6, 12.4 Hz, 2H), 7.23- |
| | | | 7.12 (m, 4H), 6.99 (d, J = 8.8 Hz, 1H), 5.10-4.86 (m, 2H), 4.34 (dd, J = |
| | | | 5.2, 10.0 Hz, 1H), 4.07 (t, J = 6.0 Hz, 2H), 3.98-3.89 (m, 5H), 3.07- |
| | | | 3.00 (m, 2H), 2.73-2.55 (m, 4H), 2.43-2.28 (m, 2H), 2.24-2.11 (m, |
| | | | 1H), 1.93-1.55 (m, 7H), 1.52-0.93 (m, 7H). |
| 95 | 209c | m/z = 1028.3 | ¹H NMR (400 MHz, DMSO-d₆) δ = 10.88 (s, 1H), 9.81 (s, 1H), 8.26 (d, |
| | | [M + H]⁺ | J = 8.8 Hz, 2H), 7.78 (d, J = 8.0 Hz, 1H), 7.67-7.59 (m, 2H), 7.49- |
| | | | 7.41 (m, 3H), 7.40-7.32 (m, 2H), 7.23 (dd, J = 1.6, 8.8 Hz, 1H), 7.18 |
| | | | (d, J = 9.2 Hz, 3H), 6.97 (d, J = 8.8 Hz, 1H), 5.07-4.93 (m, 2H), 4.33 |
| | | | (dd, J = 4.8, 9.6 Hz, 1H), 4.05 (t, J = 6.0 Hz, 2H), 3.97-3.90 (m, 5H), |
| | | | 3.08 (d, J = 16.4 Hz, 2H), 3.03 (t, J = 6.0 Hz, 2H), 2.92 (d, J = 11.6 Hz, |
| | | | 1H), 2.69-2.59 (m, 2H), 2.45-2.30 (m, 3H), 2.21-2.13 (m, 1H), 1.81- |
| | | | 1.74 (m, 2H), 1.67 (d, J = 12.0 Hz, 2H), 1.43-1.33 (m, 3H), 1.30- |
| | | | 1.22 (m, 1H), 1.05 (d, J = 6.4 Hz, 3H), 1.04-0.95 (m, 1H) |
| 96 | 213 | m/z = 480.9 | ¹H NMR (400 MHz, DMSO-d₆) δ = 12.83-12.10 (m, 2H), 10.93 (s, |
| | | [M/2 + H]⁺ | 1H), 10.26-10.02 (m, 1H), 8.58 (d, J = 4.8 Hz, 1H), 8.21 (d, J = 8.8 |
| | | | Hz, 1H), 8.09-7.99 (m, 2H), 7.82 (d, J = 7.2 Hz, 1H), 7.59 (d, J = 4.8 |
| | | | Hz, 1H), 7.54-7.47 (m, 2H), 7.41-7.34 (m, 1H), 7.06 (d, J = 8.8 Hz, |
| | | | 1H), 6.94 (d, J = 8.4 Hz, 1H), 6.73 (d, J = 2.0 Hz, 1H), 6.65 (dd, J = 2.0, |
| | | | 8.4 Hz, 1H), 5.25 (s, 2H), 4.74 (t, J = 6.8 Hz, 1H), 4.35 (dd, J = 4.8, |
| | | | 10.2 Hz, 1H), 4.00-3.94 (m, 5H), 3.23 (s, 2H), 3.08 (t, J = 5.2 Hz, 2H), |
| | | | 2.76-2.57 (m, 4H), 2.44-2.33 (m, 4H), 2.27-2.12 (m, 2H), 2.05 (s, |
| | | | 3H), 1.81 (dd, J = 6.8, 12.0 Hz, 2H), 1.68 (dd, J = 4.8, 13.2 Hz, 4H). |
| 97 | 214 | m/z = 516.3 | ¹H NMR (400 MHz, DMSO-d₆) δ = 12.61-12.51 (m, 1H), 12.31 (s, |
| | | [M/2 + H]⁺ | 1H), 10.92 (s, 1H), 9.76-9.70 (m, 1H), 8.57 (d, J = 5.2 Hz, 1H), 8.05 |
| | | | (d, J = 7.6 Hz, 1H), 7.82 (d, J = 8.4 Hz, 1H), 7.59 (d, J = 5.2 Hz, 2H), |
| | | | 7.51-7.46 (m, 4H), 7.39-7.34 (m, 1H), 7.08 (d, J = 8.8 Hz, 1H), 7.03 |
| | | | (d, J = 8.4 Hz, 1H), 6.70 (d, J = 7.2 Hz, 1H), 5.31-5.20 (m, 2H), 4.94- |
| | | | 4.88 (m, 1H), 4.38 (dd, J = 4.8, 10.0 Hz, 1H), 4.11 (s, 3H), 4.03-3.96 |
| | | | (m, 2H), 3.15 (s, 2H), 3.08 (t, J = 5.6 Hz, 2H), 2.72-2.65 (m, 2H), 2.64- |
| | | | 2.58 (m, 2H), 2.37-2.31 (m, 2H), 2.23-2.12 (m, 2H), 1.85-1.80 (m, |
| | | | 2H), 1.76-1.63 (m, 6H). |
| 98 | 215a | m/z = 1028.4 | ¹H NMR (400 MHz, DMSO-d₆) δ = 13.06-12.03 (m, 2H), 10.88 (s, |
| | | [M + H]⁺ | 1H), 9.82 (s, 1H), 8.07-8.00 (m, 2H), 7.79 (d, J = 8.0 Hz, 1H), 7.67- |
| | | | 7.58 (m, 2H), 7.51-7.44 (m, 3H), 7.42-7.32 (m, 3H), 7.25-7.16 (m, |
| | | | 2H), 6.99 (d, J = 8.8 Hz, 1H), 6.67 (d, J = 7.6 Hz, 1H), 5.06-4.93 (m, |
| | | | 2H), 4.32 (dd, J = 5.2, 10.0 Hz, 1H), 4.13-4.06 (m, 2H), 3.97-3.97 |
| | | | (m, 1H), 3.97-3.89 (m, 4H), 3.24-3.26 (m, 1H), 3.12 (d, J = 15.6 Hz, |
| | | | 1H), 3.03 (t, J = 5.2 Hz, 2H), 2.68-2.59 (m, 4H), 2.40-2.29 (m, 2H), |
| | | | 2.21-2.14 (m, 1H), 1.80-1.72 (m, 2H), 1.70-1.60 (m, 2H), 1.54 (s, |
| | | | 2H), 1.46-1.39 (m, 2H), 1.34-1.25 (m, 1H), 1.02 (d, J = 6.4 Hz, 3H). |
| 99 | 215b | m/z = 1028.3 | ¹H NMR (400 MHz, DMSO-d₆) δ = 13.01-12.69 (m, 1H), 12.66- |
| | | [M + H]⁺ | 12.26 (m, 1H), 10.88 (s, 1H), 9.80 (s, 1H), 8.03 (d, J = 6.0 Hz, 2H), 7.79 |
| | | | (d, J = 8.0 Hz, 1H), 7.65-7.60 (m, 2H), 7.51-7.43 (m, 3H), 7.42-7.34 |
| | | | (m, 3H), 7.26-7.18 (m, 2H), 6.99 (d, J = 8.4 Hz, 1H), 6.67 (d, J = 7.6 |
| | | | Hz, 1H), 5.05-4.93 (m, 2H), 4.32 (dd, J = 4.8, 9.6 Hz, 1H), 4.09 (d, J = |
| | | | 3.6 Hz, 2H), 3.96-3.91 (m, 5H), 3.07-2.88 (m, 4H), 2.71-2.54 (m, |
| | | | 4H), 2.44-2.29 (m, 4H), 2.21-2.14 (m, 1H), 1.76 (s, 2H), 1.65 (d, J = |
| | | | 10.4 Hz, 2H), 1.39 (d, J = 5.2 Hz, 2H), 1.29-1.21 (m, 1H), 1.05 (d, J = |
| | | | 6.0 Hz, 3H) |
| 100 | 216a | m/z = 975.7 | ¹H NMR (400 MHz, DMSO-d₆) δ = 12.39-12.17 (m, 1H), 10.89 (s, |
| | | [M + H]⁺ | 1H), 9.96-9.63 (m, 1H), 8.57 (d, J = 4.8 Hz, 1H), 8.12-7.89 (m, 2H), |
| | | | 7.82 (d, J = 8.0 Hz, 1H), 7.69-7.61 (m, 1H), 7.59 (d, J = 4.8 Hz, 1H), |
| | | | 7.53-7.44 (m, 2H), 7.36 (t, J = 7.2 Hz, 1H), 7.26-7.16 (m, 1H), 7.15- |
| | | | 7.01 (m, 2H), 6.90 (d, J = 8.8 Hz, 1H), 6.65 (d, J = 7.6 Hz, 1H), 5.25 (s, |
| | | | 2H), 4.33 (dd, J = 5.2, 9.2 Hz, 1H), 3.97 (t, J = 5.6 Hz, 2H), 3.93 (s, |
| | | | 3H), 3.14-3.01 (m, 4H), 2.99-2.77 (m, 2H), 2.70-2.61 (m, 2H), 2.45- |
| | | | 2.24 (m, 4H), 2.19-2.14 (m, 1H), 1.92 (s, 3H), 1.80-1.75 (m, 2H), |
| | | | 1.73-1.60 (m, 2H), 1.45-1.35 (m, 3H), 1.33-1.19 (m, 2H), 1.13- |
| | | | 0.98 (m, 3H) |
| 101 | 215c | m/z = 1028.4 | ¹H NMR (400 MHz, DMSO-d₆) δ = 12.90-12.30 (m, 2H), 10.89 (s, |
| | | [M + H]⁺ | 1H), 9.98-9.62 (m, 1H), 8.04-8.02 (m, 2H), 7.79 (d, J = 8.0 Hz, 1H), |
| | | | 7.66-7.62 (m, 2H), 7.52-7.45 (m, 3H), 7.42-7.33 (m, 3H), 7.20 (d, J = |
| | | | 8.8 Hz, 2H), 7.00 (d, J = 8.4 Hz, 1H), 6.68 (d, J = 8.0 Hz, 1H), 5.05- |
| | | | 4.92 (m, 2H), 4.35-4.31 (m, 1H), 4.11 (t, J = 6.0 Hz, 2H), 3.96-3.93 |
| | | | (m, 5H), 3.27-3.24 (m, 2H), 3.03 (t, J = 5.6 Hz, 2H), 2.65-2.58 (m, |
| | | | 2H), 2.46-2.36 (m, 2H), 2.20-2.15 (m, 2H), 1.83-1.72 (m, 3H), 1.71- |
| | | | 1.50 (m, 4H), 1.48-1.26 (m, 4H), 1.10-0.98 (m, 2H) |
| 102 | 217 | m/z = 959.4 | ¹H NMR (400 MHz, DMSO-d₆) δ = 13.30-12.61 (m, 1H), 11.12- |
| | | [M + H]⁺ | 10.74 (m, 1H), 10.66-9.53 (m, 1H), 8.13 (s, 1H), 8.09-7.99 (m, 2H), |
| | | | 7.79 (d, J = 8.0 Hz, 1H), 7.64 (t, J = 9.6 Hz, 2H), 7.50-7.31 (m, 5H), |
| | | | 7.25-7.15 (m, 1H), 7.08-6.90 (m, 2H), 6.55-6.44 (m, 1H), 6.31 (d, J = |
| | | | 7.6 Hz, 1H), 4.97 (s, 2H), 4.87-4.69 (m, 1H), 4.33 (dd, J = 5.2, 10.0 |
| | | | Hz, 1H), 3.92 (s, 3H), 3.92-3.87 (m, 2H), 3.13-2.95 (m, 6H), 2.82- |
| | | | 2.56 (m, 6H), 2.41-2.27 (m, 2H), 2.21-2.15 (m, 1H), 1.80 (s, 3H), |
| | | | 1.76-1.61 (m, 4H), 1.34 (s, 4H). |
| 103 | 218 | m/z = 959.4 | ¹H NMR (400 MHz, DMSO-d₆) δ = 13.10-12.69 (m, 1H), 10.88 (s, |
| | | [M + H]⁺ | 1H), 9.99-9.74 (m, 1H), 8.14 (s, 1H), 8.06-8.01 (m, 2H), 7.79 (d, J = |
| | | | 8.0 Hz, 1H), 7.65-7.60 (m, 2H), 7.48-7.33 (m, 5H), 7.21 (d, J = 8.8 |
| | | | Hz, 1H), 6.91 (d, J = 9.2 Hz, 1H), 6.73 (d, J = 8.0 Hz, 1H), 6.39 (s, 1H), |
| | | | 6.33 (d, J = 8.4 Hz, 1H), 5.63-5.36 (m, 1H), 4.95 (s, 2H), 4.32 (dd, J = |
| | | | 4.8, 9.6 Hz, 1H), 3.94-3.91 (m, 3H), 3.89 (t, J = 5.6 Hz, 2H), 3.22- |
| | | | 3.13 (m, 2H), 3.03-2.96 (m, 4H), 2.94-2.81 (m, 2H), 2.73-2.54 (m, |
| | | | 3H), 2.37-2.31 (m, 1H), 2.21-2.14 (m, 2H), 1.96 (s, 3H), 1.71-1.53 |
| | | | (m, 4H), 1.34-1.23 (m, 5H). |
| 104 | 216b | m/z = 975.4 | ¹H NMR (400 MHz, DMSO-d₆) δ = 12.55-12.14 (m, 1H), 10.89 (s, |
| | | [M + H]⁺ | 1H), 9.83 (s, 1H), 8.60-8.55 (m, 1H), 8.08-8.04 (m, 2H), 7.82 (d, J = |
| | | | 8.0 Hz, 1H), 7.64 (d, J = 9.2 Hz, 1H), 7.59 (d, J = 4.8 Hz, 1H), 7.53- |
| | | | 7.47 (m, 2H), 7.40-7.34 (m, 1H), 7.23 (d, J = 8.4 Hz, 1H), 7.14-7.06 |
| | | | (m, 2H), 6.91 (d, J = 8.4 Hz, 1H), 6.66 (d, J = 7.6 Hz, 1H), 5.26 (s, 2H), |
| | | | 4.33 (dd, J = 5.2, 9.6 Hz, 1H), 3.99 (t, J = 5.6 Hz, 4H), 3.93 (s, 3H), |
| | | | 3.27 (s, 2H), 3.09 (t, J = 6.4 Hz, 2H), 2.69-2.60 (m, 4H), 2.34 (s, 1H), |
| | | | 2.18 (dd, J = 6.0, 13.2 Hz, 1H), 1.93 (s, 3H), 1.81-1.76 (m, 2H), 1.72- |
| | | | 1.65 (m, 2H), 1.56 (s, 2H), 1.47-1.42 (m, 2H), 1.39-1.23 (m, 2H), |
| | | | 1.03 (d, J = 6.4 Hz, 3H). |
| 105 | 219a | m/z = 975.2 | ¹H NMR (400 MHz, DMSO-d₆) δ = 12.81-12.60 (m, 1H), 12.35- |
| | | [M + H]⁺ | 12.25 (m, 1H), 10.89 (s, 1H), 9.93-9.75 (m, 1H), 8.58 (d, J = 4.8 Hz, |
| | | | 1H), 8.08-8.02 (m, 2H), 7.82 (d, J = 8.0 Hz, 1H), 7.68-7.63 (m, 1H), |
| | | | 7.59 (d, J = 4.8 Hz, 1H), 7.53-7.47 (m, 2H), 7.40-7.34 (m, 1H), 7.26- |
| | | | 7.18 (m, 1H), 7.06 (d, J = 8.8 Hz, 1H), 6.95 (d, J = 8.0 Hz, 1H), 6.82 (s, |
| | | | 1H), 6.74 (dd, J = 2.0, 8.8 Hz, 1H), 5.25 (s, 2H), 4.34 (dd, J = 5.2, 9.6 |
| | | | Hz, 1H), 3.99-3.92 (m, 7H), 3.10-3.07 (m, 2H), 2.68-2.58 (m, 5H), |
| | | | 2.39-2.30 (m, 3H), 2.21-2.17 (m, 1H), 2.06 (s, 3H), 1.97-1.85 (m, |
| | | | 1H), 1.80-1.67 (m, 5H), 1.57 (d, J = 2.8 Hz, 1H), 1.43-1.38 (m, 2H), |
| | | | 1.09-0.98 (m, 3H). |
| 106 | 216c | m/z = 488.3 | ¹H NMR (400 MHz, DMSO-d₆) δ = 12.79-12.58 (m, 1H), 12.33 (s, |
| | | [M/2 + H]⁺ | 1H), 10.90 (s, 1H), 8.58 (d, J = 4.8 Hz, 1H), 8.09-8.02 (m, 2H), 7.82 |
| | | | (d, J = 8.0 Hz, 1H), 7.72-7.65 (m, 1H), 7.59 (d, J = 5.2 Hz, 1H), 7.52- |
| | | | 7.47 (m, 2H), 7.40-7.34 (m, 1H), 7.13-7.09 (m, 3H), 6.91 (d, J = 8.0 |
| | | | Hz, 1H), 6.66 (d, J = 7.6 Hz, 1H), 5.26 (s, 2H), 4.34-4.32 (m, 1H), |
| | | | 3.99 (s, 4H), 3.94 (s, 3H), 3.27 (s, 2H), 3.09 (t, J = 5.6 Hz, 2H), 2.65- |
| | | | 2.63 (m, 2H), 2.36-2.35 (m, 2H), 2.22-2.09 (m, 2H), 1.93 (s, 3H), |
| | | | 1.78 (d, J = 5.2 Hz, 5H), 1.63-1.49 (m, 2H), 1.43-1.41 (m, 3H), 1.34- |
| | | | 1.25 (m, 2H), 1.11-0.98 (m, 1H). |
| 107 | 219c | m/z = 488.2 | ¹H NMR (400 MHz, DMSO-d₆) δ = 12.77-12.51 (m, 1H), 12.39- |
| | | [M/2 + H]⁺ | 12.20 (m, 1H), 10.88 (s, 1H), 9.88-9.77 (m, 1H), 8.57 (d, J = 4.8 Hz, |
| | | | 1H), 8.07-8.03 (m, 2H), 7.82 (d, J = 8.0 Hz, 1H), 7.64 (d, J = 9.2 Hz, |
| | | | 1H), 7.58 (d, J = 5.2 Hz, 1H), 7.50 (d, J = 8.4 Hz, 2H), 7.40-7.33 (m, |
| | | | 1H), 7.23 (dd, J = 1.6, 8.4 Hz, 1H), 7.05 (d, J = 8.4 Hz, 1H), 6.94 (d, J = |
| | | | 8.4 Hz, 1H), 6.81 (d, J = 2.4 Hz, 1H), 6.75-6.69 (m, 1H), 5.24 (s, 2H), |
| | | | 4.37-4.29 (m, 1H), 3.98-3.94 (m, 4H), 3.93 (s, 3H), 3.12-3.02 (m, |
| | | | 4H), 2.67-2.62 (m, 2H), 2.36-2.29 (m, 2H), 2.23-2.09 (m, 2H), 2.05 |
| | | | (s, 3H), 1.76-1.65 (m, 4H), 1.57-1.52 (m, 2H), 1.43-1.30 (m, 4H), |
| | | | 1.03 (d, J = 6.4 Hz, 3H). |
| 108 | 1001b | m/z = 486.9 | ¹H NMR (400 MHz, DMSO-d₆) δ = 13.27-12.35 (m, 1H), 10.89 (s, |
| | | [M/2 + H]⁺ | 1H), 10.02-9.67 (m, 1H), 8.15 (s, 1H), 8.06-8.00 (m, 2H), 7.79 (d, J = |
| | | | 8.0 Hz, 1H), 7.63 (d, J = 8.8 Hz, 2H), 7.49-7.42 (m, 3H), 7.40-7.33 |
| | | | (m, 2H), 7.24-7.19 (m, 1H), 7.08 (t, J = 7.6 Hz, 1H), 6.95 (d, J = 8.8 |
| | | | Hz, 1H), 6.85 (d, J = 8.4 Hz, 1H), 6.62 (d, J = 7.6 Hz, 1H), 4.98 (s, 2H), |
| | | | 4.32 (dd, J = 5.2, 9.6 Hz, 1H), 4.00 (dd, J = 6.0, 10.4 Hz, 1H), 3.94- |
| | | | 3.89 (m, 5H), 3.71-3.65 (m, 1H), 3.15 (s, 2H), 3.02 (t, J = 5.6 Hz, 2H), |
| | | | 2.88 (d, J = 10.4 Hz, 2H), 2.69-2.60 (m, 2H), 2.34 (dd, J = 5.6, 8.4 Hz, |
| | | | 1H), 2.19-2.09 (m, 3H), 1.92 (s, 3H), 1.75-1.66 (m, 2H), 1.48-1.38 |
| | | | (m, 2H), 1.10-1.01 (m, 1H), 0.75-0.62 (m, 2H), 0.54-0.42 (m, 2H). |
| 109 | 1001a | m/z = 972.2 | ¹H NMR (400 MHz, DMSO-d₆) δ = 13.02-12.69 (m, 1H), 10.88 (s, |
| | | [M + H]⁺ | 1H), 10.17-9.66 (m, 1H), 8.13 (s, 1H), 8.08-7.98 (m, 2H), 7.78 (d, J = |
| | | | 7.8 Hz, 1H), 7.62 (dd, J = 4.8, 8.0 Hz, 2H), 7.51-7.41 (m, 3H), 7.40- |
| | | | 7.31 (m, 2H), 7.20 (d, J = 8.8 Hz, 1H), 7.07 (t, J = 7.8 Hz, 1H), 6.95 (d, |
| | | | J = 8.8 Hz, 1H), 6.84 (d, J = 8.0 Hz, 1H), 6.62 (d, J = 7.6 Hz, 1H), 5.07- |
| | | | 4.88 (m, 2H), 4.32 (dd, J = 5.2, 9.6 Hz, 1H), 4.06-3.98 (m, 1H), 3.95- |
| | | | 3.86 (m, 5H), 3.73-3.60 (m, 2H), 3.02 (t, J = 5.6 Hz, 3H), 2.95-2.84 |
| | | | (m, 2H), 2.66-2.59 (m, 2H), 2.40-2.27 (m, 2H), 2.20-2.11 (m, 2H), |
| | | | 1.92 (s, 3H), 1.79-1.64 (m, 2H), 1.56-1.38 (m, 2H), 1.05 (dd, J = 4.8, |
| | | | 10.4 Hz, 1H), 0.80-0.59 (m, 2H), 0.56-0.41 (m, 2H). |
| 110 | 1001c | m/z = 972.5 | ¹H NMR (400 MHz, DMSO-d₆) δ = 12.94-12.80 (m, 1H), 10.89 (s, |
| | | [M + H]⁺ | 1H), 9.95-9.66 (m, 1H), 8.08-7.98 (m, 2H), 7.78 (d, J = 8.0 Hz, 1H), |
| | | | 7.69-7.58 (m, 2H), 7.49-7.43 (m, 3H), 7.40-7.32 (m, 2H), 7.27- |
| | | | 7.14 (m, 1H), 7.09 (t, J = 8.0 Hz, 1H), 6.96 (d, J = 8.8 Hz, 1H), 6.91 (d, |
| | | | J = 8.0 Hz, 1H), 6.69-6.58 (m, 1H), 5.04-4.93 (m, 2H), 4.35-4.30 |
| | | | (m, 1H), 4.17-4.10 (m, 1H), 3.95-3.87 (m, 6H), 3.24-3.09 (m, 2H), |
| | | | 3.03 (d, J = 6.0 Hz, 2H), 2.97-2.73 (m, 2H), 2.72-2.54 (m, 5H), 2.46- |
| | | | 2.27 (m, 2H), 2.22-2.10 (m, 2H), 1.91 (s, 3H), 1.79-1.67 (m, 1H), |
| | | | 1.55-1.46 (m, 1H), 1.37-1.29 (m, 1H), 0.87-0.67 (m, 2H), 0.27- |
| | | | 0.09 (m, 1H) |
| 111 | 1001d | m/z = 972.5 | ¹H NMR (400 MHz, DMSO-d₆) δ = 12.97-12.80 (m, 1H), 10.91- |
| | | [M + H]⁺ | 10.87 (m, 1H), 10.08-9.68 (m, 1H), 8.13 (s, 1H), 8.06-8.01 (m, 2H), |
| | | | 7.78 (d, J = 8.0 Hz, 1H), 7.67-7.61 (m, 2H), 7.48-7.43 (m, 3H), 7.39- |
| | | | 7.31 (m, 2H), 7.29-7.15 (m, 1H), 7.13-7.07 (m, 1H), 6.96 (d, J = 8.8 |
| | | | Hz, 1H), 6.91 (d, J = 8.0 Hz, 1H), 6.63 (d, J = 7.6 Hz, 1H), 4.97 (s, 2H), |
| | | | 4.33 (dd, J = 5.2, 10.0 Hz, 1H), 4.15-4.09 (m, 1H), 3.94-3.89 (m, |
| | | | 6H), 3.25-3.08 (m, 2H), 3.02 (t, J = 6.0 Hz, 2H), 3.00-2.71 (m, 2H), |
| | | | 2.70-2.53 (m, 5H), 2.48-2.26 (m, 2H), 2.23-2.11 (m, 2H), 1.91 (s, |
| | | | 3H), 1.80-1.68 (m, 1H), 1.55-1.48 (m, 1H), 1.37-1.30 (m, 1H), 0.81- |
| | | | 0.70 (m, 2H), 0.21-0.13 (m, 1H) |
| 112 | 1003b | m/z = 972.3 | ¹H NMR (400 MHz, DMSO-d₆) δ = 13.02-12.71 (m, 1H), 10.88 (s, |
| | | [M + H]⁺ | 1H), 9.90 (s, 1H), 8.07-7.98 (m, 2H), 7.78 (d, J = 7.6 Hz, 1H), 7.67- |
| | | | 7.58 (m, 2H), 7.51-7.41 (m, 3H), 7.39-7.31 (m, 2H), 7.20 (d, J = 8.4 |
| | | | Hz, 1H), 6.92 (dd, J = 8.8, 13.2 Hz, 2H), 6.77 (s, 1H), 6.69 (dd, J = 2.4, |
| | | | 8.4 Hz, 1H), 4.96 (s, 2H), 4.32 (dd, J = 5.2, 10.0 Hz, 1H), 3.93-3.88 |
| | | | (m, 5H), 3.84-3.77 (m, 2H), 3.02 (t, J = 6.0 Hz, 3H), 2.95-2.88 (m, |
| | | | 2H), 2.68-2.61 (m, 2H), 2.38-2.30 (m, 2H), 2.24-2.11 (m, 3H), 2.02 |
| | | | (s, 3H), 1.76-1.66 (m, 2H), 1.48-1.34 (m, 2H), 1.06-0.96 (m, 1H), |
| | | | 0.78-0.60 (m, 2H), 0.50-0.38 (m, 2H). |
| 113 | 1004a | m/z = 986.2 | ¹H NMR (400 MHz, DMSO-d₆) δ = 10.89 (s, 1H), 9.82 (s, 1H), 8.16 (s, |
| | | [M + H]⁺ | 1H), 8.06-7.95 (m, 2H), 7.78 (d, J = 8.0 Hz, 1H), 7.66-7.58 (m, 2H), |
| | | | 7.49-7.31 (m, 5H), 7.19 (d, J = 8.8 Hz, 1H), 7.07 (t, J = 8.0 Hz, 1H), |
| | | | 6.93 (d, J = 8.8 Hz, 1H), 6.83 (d, J = 8.4 Hz, 1H), 6.62 (d, J = 7.6 Hz, |
| | | | 1H), 4.97 (s, 2H), 4.31 (d, J = 9.6, 5.2 Hz, 1H), 4.09-4.00 (m, 1H), |
| | | | 3.91 (s, 5H), 3.63-3.58 (m, 1H), 3.10 (s, 2H), 3.01 (t, J = 5.2 Hz, 2H), |
| | | | 2.86 (d, J = 10.8 Hz, 2H), 2.68-2.58 (m, 2H), 2.37-2.29 (m, 1H), 2.19- |
| | | | 2.07 (m, 3H), 1.90 (s, 3H), 1.88-1.82 (m, 1H), 1.77-1.67 (m, 1H), |
| | | | 1.45 (d, J = 13.2, 5.6 Hz, 1H), 1.39-1.22 (m, 3H), 1.05-0.92 (m, 2H), |
| | | | 0.84-0.73 (m, 1H), 0.56-0.46 (m, 1H), 0.36 (td, J = 8.4, 4.4 Hz, 1H). |
| 114 | 1002a | m/z = 986.2 | ¹H NMR (400 MHz, DMSO-d₆) δ = 10.89 (s, 1H), 9.87 (s, 1H), 8.19 (s, |
| | | [M + H]⁺ | 1H), 8.06-7.98 (m, 2H), 7.78 (d, J = 8.0 Hz, 1H), 7.62 (d, J = 8.4 Hz, |
| | | | 2H), 7.49-7.41 (m, 2H), 7.41-7.31 (m, 3H), 7.21 (d, J = 8.8, 1.6 Hz, |
| | | | 1H), 6.95-6.87 (m, 2H), 6.75 (d, J = 2.4 Hz, 1H), 6.67 (d, J = 8.4, 2.0 |
| | | | Hz, 1H), 4.96 (s, 2H), 4.32 (d, J = 9.6, 5.2 Hz, 1H), 3.93-3.87 (m, 5H), |
| | | | 3.87-3.82 (m, 1H), 3.77 (d, J = 10.0, 7.2 Hz, 1H), 3.14 (s, 2H), 3.01 (t, |
| | | | J = 5.6 Hz, 2H), 2.88 (d, J = 10.4 Hz, 2H), 2.63-2.59 (m, 1H), 2.33 (d, |
| | | | J = 18.0, 10.0, 5.2 Hz, 2H), 2.19-2.11 (m, 3H), 2.01 (s, 3H), 1.82- |
| | | | 1.68 (m, 2H), 1.36-1.24 (m, 4H), 1.16-1.08 (m, 1H), 1.01-0.92 (m, |
| | | | 1H), 0.80-0.70 (m, 1H), 0.54-0.45 (m, 1H), 0.39-0.31 (m, 1H). |
| 115 | 220 | m/z = 973.4 | ¹H NMR (400 MHz, DMSO-d₆) δ = 13.12-12.63 (m, 1H), 10.88 (s, |
| | | [M + H]⁺ | 1H), 10.24-9.69 (m, 1H), 8.13 (s, 1H), 8.06-7.99 (m, 2H), 7.78 (d, J = |
| | | | 8.0 Hz, 1H), 7.63 (dd, J = 5.6, 8.4 Hz, 2H), 7.49-7.43 (m, 3H), 7.39- |
| | | | 7.32 (m, 2H), 7.19 (d, J = 8.8 Hz, 1H), 7.12-7.07 (m, 1H), 7.03-6.99 |
| | | | (m, 1H), 6.96 (d, J = 8.8 Hz, 1H), 6.72 (d, J = 7.6 Hz, 1H), 4.98 (s, 2H), |
| | | | 4.32 (dd, J = 4.8, 9.6 Hz, 1H), 3.93-3.89 (m, 5H), 3.02 (t, J = 5.6 Hz, |
| | | | 4H), 2.81 (s, 2H), 2.68-2.61 (m, 2H), 2.59 (s, 4H), 2.41-2.28 (m, 2H), |
| | | | 2.26-2.10 (m, 2H), 1.99 (s, 3H), 1.65 (s, 2H), 1.51 (d, J = 1.2 Hz, 2H), |
| | | | 1.25 (d, J = 4.8 Hz, 6H). |
| 116 | 1002b | m/z = 986.4 | ¹H NMR (400 MHz, DMSO-d₆) δ = 13.3-12.1 (m, 2H), 10.89 (s, 1H), |
| | | [M + H]⁺ | 10.30-9.63 (m, 1H), 8.10-7.95 (m, 2H), 7.79 (d, J = 8.0 Hz, 1H), |
| | | | 7.68-7.58 (m, 2H), 7.51-7.40 (m, 3H), 7.40-7.31 (m, 2H), 7.18 (d, J = |
| | | | 8.8 Hz, 1H), 6.95 (d, J = 8.8 Hz, 1H), 6.91 (d, J = 8.4 Hz, 1H), 6.81 |
| | | | (d, J = 2.4 Hz, 1H), 6.72 (dd, J = 2.4, 8.4 Hz, 1H), 4.97 (s, 2H), 4.37- |
| | | | 4.28 (m, 1H), 4.09 (dd, J = 6.4, 10.4 Hz, 1H), 3.98-3.86 (m, 5H), 3.85- |
| | | | 3.79 (m, 1H), 3.11-2.89 (m, 4H), 2.75-2.53 (m, 4H), 2.41-2.27 (m, |
| | | | 2H), 2.20-2.14 (m, 1H), 2.03 (s, 3H), 1.88-1.78 (m, 1H), 1.77-1.67 |
| | | | (m, 1H), 1.60-1.08 (m, 7H), 1.00-0.89 (m, 1H), 0.84-0.72 (m, 1H), |
| | | | 0.18-0.60 (m, 1H) |
| 117 | 219d | m/z = 488.4 | ¹H NMR (400 MHz, DMSO-d₆) δ = 12.84-12.46 (m, 1H), 12.43- |
| | | [M/2 + H]⁺ | 12.17 (m, 1H), 10.89 (s, 1H), 9.93-9.60 (m, 1H), 8.58 (d, J = 4.8 Hz, |
| | | | 1H), 8.10-7.99 (m, 2H), 7.82 (d, J = 8.0 Hz, 1H), 7.71-7.63 (m, 1H), |
| | | | 7.59 (d, J = 5.2 Hz, 1H), 7.55-7.42 (m, 2H), 7.37 (t, J = 7.6 Hz, 1H), |
| | | | 7.26-7.15 (m, 1H), 7.06 (d, J = 8.8 Hz, 1H), 6.95 (d, J = 8.4 Hz, 1H), |
| | | | 6.81 (d, J = 2.0 Hz, 1H), 6.77-6.69 (m, 1H), 5.25 (s, 2H), 4.34 (dd, J = |
| | | | 5.2, 10.4 Hz 1H), 4.00-3.93 (m, 7H), 3.39-3.35 (m, 2H), 3.09 (t, J = |
| | | | 5.6 Hz, 2H), 2.68-2.61 (m, 2H), 2.57 (s, 4H), 2.44-2.30 (m, 2H), 2.22- |
| | | | 2.15 (m, 1H), 2.05 (s, 3H), 1.80-1.63 (m, 4H), 1.44-1.28 (m, 4H), |
| | | | 1.10-0.99 (m, 2H) |
| 118 | 1003c | m/z = 972.4 | ¹H NMR (400 MHz, DMSO-d₆) δ = 12.99-12.76 (m, 1H), 10.89 (s, |
| | | [M + H]⁺ | 1H), 10.06-9.67 (m, 1H), 8.13 (s, 1H), 8.06-8.01 (m, 2H), 7.79 (d, J = |
| | | | 8.0 Hz, 1H), 7.63 (t, J = 8.4 Hz, 2H), 7.50-7.42 (m, 3H), 7.39-7.33 |
| | | | (m, 2H), 7.19 (d, J = 8.4 Hz, 1H), 6.93 (dd, J = 8.8, 15.2 Hz, 2H), 6.80 |
| | | | (d, J = 2.4 Hz, 1H), 6.72 (dd, J = 2.4, 8.4 Hz, 1H), 4.97 (s, 2H), 4.33 |
| | | | (dd, J = 5.2, 9.6 Hz, 1H), 4.13 (dd, J = 6.4, 8.8 Hz, 1H), 3.95-3.88 (m, |
| | | | 5H), 3.83 (t, J = 9.6 Hz, 1H), 3.24-3.10 (m, 2H), 3.02 (t, J = 5.6 Hz, |
| | | | 2H), 2.98-2.75 (m, 2H), 2.69-2.60 (m, 2H), 2.42-2.25 (m, 2H), 2.23- |
| | | | 2.10 (m, 2H), 2.02 (s, 3H), 1.99-1.87 (m, 1H), 1.81-1.67 (m, 1H), |
| | | | 1.63-1.42 (m, 2H), 1.36-1.26 (m, 1H), 1.10-0.88 (m, 1H), 0.83- |
| | | | 0.68 (m, 2H), 0.24-0.06 (m, 1H). |
| 119 | 1003d | m/z = 972.4 | ¹H NMR (400 MHz, DMSO-d₆) δ = 12.96-12.43 (m, 1H), 10.88 (s, |
| | | [M + H]⁺ | 1H), 9.84 (s, 1H), 8.16 (s, 1H), 8.07-8.00 (m, 2H), 7.79 (d, J = 8.0 Hz, |
| | | | 1H), 7.63 (d, J = 8.8 Hz, 2H), 7.49-7.42 (m, 3H), 7.39-7.32 (m, 2H), |
| | | | 7.21 (dd, J = 1.2, 8.8 Hz, 1H), 6.96-6.89 (m, 2H), 6.80 (d, J = 2.4 Hz, |
| | | | 1H), 6.72 (dd, J = 2.4, 8.4 Hz, 1H), 4.97 (s, 2H), 4.32 (dd, J = 5.2, 9.6 |
| | | | Hz, 1H), 4.10 (dd, J = 5.6, 10.4 Hz, 1H), 3.93-3.88 (m, 5H), 3.84 (t, J = |
| | | | 9.6 Hz, 1H), 3.12 (d, J = 2.0 Hz, 2H), 3.02 (t, J = 5.6 Hz, 2H), 2.92- |
| | | | 2.82 (m, 2H), 2.68-2.60 (m, 2H), 2.38-2.31 (m, 1H), 2.22-2.08 (m, |
| | | | 3H), 2.03 (s, 3H), 1.93-1.85 (m, 1H), 1.75-1.67 (m, 1H), 1.55-1.42 |
| | | | (m, 2H), 1.33-1.21 (m, 1H), 0.99-0.86 (m, 1H), 0.83-0.68 (m, 2H), |
| | | | 0.20-0.07 (m, 1H). |
| 120 | 1004b | m/z = 493.9 | ¹H NMR (400 MHz, DMSO-d₆) δ = 10.88 (s, 1H), 9.82 (s, 1H), 8.21 (s, |
| | | [M/2 + H]⁺ | 1H), 8.08-7.95 (m, 2H), 7.78 (d, J = 8.0 Hz, 1H), 7.66-7.56 (m, 2H), |
| | | | 7.50-7.28 (m, 5H), 7.19 (d, J = 9.2 Hz, 1H), 7.06 (t, J = 7.6 Hz, 1H), |
| | | | 6.89 (d, J = 8.4 Hz, 1H), 6.82 (d, J = 8.4 Hz, 1H), 6.63 (d, J = 7.6 Hz, |
| | | | 1H), 4.97 (s, 2H), 4.31 (dd, J = 5.2, 9.4 Hz, 1H), 4.04 (dd, J = 6.4, 9.6 |
| | | | Hz, 1H), 3.91 (s, 5H), 3.60 (s, 1H), 3.10 (s, 2H), 3.01 (t, J = 5.6 Hz, |
| | | | 2H), 2.89-2.83 (m, 2H), 2.63 (dd, J = 4.0, 9.2 Hz, 2H), 2.50-2.30 (m, |
| | | | 1H), 2.20-2.05 (m, 3H), 1.91 (s, 3H), 1.85 (d, J = 11.5 Hz, 1H), 1.72 |
| | | | (d, J = 11.0 Hz, 1H), 1.49-1.41 (m, 1H), 1.39-1.22 (m, 3H), 1.07- |
| | | | 0.91 (m, 2H), 0.78 (d, J = 4.4 Hz, 1H), 0.51 (dd, J = 4.4, 8.0 Hz, 1H), |
| | | | 0.36 (dd, J = 4.0, 8.0 Hz, 1H) |
| 121 | 1004d | m/z = 494.1 | ¹H NMR (400 MHz, DMSO-d₆) δ = 13.14-12.02 (m, 2H), 10.88 (s, |
| | | [M/2 + H]⁺ | 1H), 10.20-9.54 (m, 1H), 8.13 (s, 1H), 8.08-7.98 (m, 2H), 7.78 (d, J = |
| | | | 8.0 Hz, 1H), 7.68-7.58 (m, 2H), 7.51-7.41 (m, 3H), 7.40-7.31 (m, |
| | | | 2H), 7.19 (d, J = 8.4 Hz, 1H), 7.12-7.06 (m, 1H), 6.96 (d, J = 7.6 Hz, |
| | | | 1H), 6.89 (d, J = 8.0 Hz, 1H), 6.63 (d, J = 7.2 Hz, 1H), 5.04-4.92 (m, |
| | | | 2H), 4.32 (dd, J = 5.2, 9.6 Hz, 1H), 4.17-4.06 (m, 1H), 3.91 (s, 5H), |
| | | | 3.84 (t, J = 9.3 Hz, 1H), 3.02 (t, J = 6.0 Hz, 3H), 2.97-2.88 (m, 1H), |
| | | | 2.70-2.55 (m, 3H), 2.40-2.24 (m, 2H), 2.23-2.08 (m, 2H), 1.93 (s, |
| | | | 3H), 1.86-1.77 (m, 1H), 1.74-1.65 (m, 1H), 1.56-1.13 (m, 7H), 1.01- |
| | | | 0.90 (m, 1H), 0.78 (dt, J = 4.0, 8.4 Hz, 1H), 0.15 (q, J = 4.8 Hz, 1H) |
| 122 | 1004c | m/z = 986.6 | ¹H NMR (400 MHz, DMSO-d₆) δ = 13.11-12.66 (m, 1H), 10.89 (s, |
| | | [M + H]⁺ | 1H), 10.04-9.73 (m, 1H), 8.13 (s, 1H), 8.08-7.98 (m, 2H), 7.78 (d, J = |
| | | | 8.0 Hz, 1H), 7.62 (d, J = 8.4 Hz, 2H), 7.51-7.42 (m, 3H), 7.41-7.30 |
| | | | (m, 2H), 7.24-7.15 (m, 1H), 7.12-7.05 (m, 1H), 6.95 (d, J = 6.8 Hz, |
| | | | 1H), 6.89 (d, J = 8.0 Hz, 1H), 6.63 (d, J = 7.6 Hz, 1H), 4.98 (s, 2H), |
| | | | 4.32 (dd, J = 4.8, 9.6 Hz, 1H), 4.12 (dd, J = 6.0, 10.0 Hz, 1H), 3.91 (s, |
| | | | 5H), 3.87-3.80 (m, 1H), 3.17-3.11 (m, 2H), 3.02 (t, J = 4.6 Hz, 2H), |
| | | | 2.93-2.84 (m, 2H), 2.67-2.60 (m, 2H), 2.35-2.30 (m, 1H), 2.21- |
| | | | 2.08 (m, 3H), 1.93 (s, 3H), 1.84-1.74 (m, 1H), 1.71-1.62 (m, 1H), |
| | | | 1.50-1.38 (m, 2H), 1.32-1.16 (m, 4H), 1.01-0.90 (m, 1H), 0.84- |
| | | | 0.73 (m, 1H), 0.19-0.07 (m, 1H). |
| 123 | 1002c | m/z = 493.9 | ¹H NMR (400 MHz, DMSO-d₆) δ = 10.88 (s, 1H), 9.85 (s, 1H), 8.17 (s, |
| | | [M/2 + H]⁺ | 1H), 8.13-7.93 (m, 2H), 7.78 (d, J = 8.0 Hz, 1H), 7.67-7.58 (m, 2H), |
| | | | 7.54-7.28 (m, 5H), 7.21 (d, J = 9.2 Hz, 1H), 6.89 (d, J = 8.0 Hz, 2H), |
| | | | 6.80-6.56 (m, 2H), 4.95 (s, 2H), 4.32 (dd, J = 5.2, 9.6 Hz, 1H), 3.95- |
| | | | 3.81 (m, 6H), 3.76 (dd, J = 6.0, 8.0 Hz, 1H), 3.14 (s, 2H), 3.01 (t, J = |
| | | | 5.2 Hz, 2H), 2.88 (d, J = 11.2 Hz, 2H), 2.63 (td, J = 4.8, 9.2 Hz, 2H), |
| | | | 2.37-2.29 (m, 1H), 2.19-2.13 (m, 2H), 2.01 (s, 3H), 1.81-1.66 (m, |
| | | | 2H), 1.36-1.29 (m, 3H), 1.15 (d, J = 3.6 Hz, 2H), 0.95 (s, 1H), 0.79 (d, |
| | | | J = 6.6 Hz, 2H), 0.49 (dd, J = 3.6, 7.6 Hz, 1H), 0.40-0.31 (m, 1H). |
| 124 | 1002d | m/z = 986.2 | ¹H NMR (400 MHz, DMSO-d₆) δ = 13.24 (s, 1H), 10.89 (s, 1H), 9.85 |
| | | [M + H]⁺ | (s, 1H), 8.15 (s, 1H), 8.08-8.00 (m, 2H), 7.79 (d, J = 8.0 Hz, 1H), 7.62 |
| | | | (d, J = 8.4 Hz, 2H), 7.49-7.41 (m, 3H), 7.40-7.32 (m, 2H), 7.23-7.17 |
| | | | (m, 1H), 6.98-6.88 (m, 2H), 6.80 (d, J = 2.0 Hz, 1H), 6.72 (dd, J = 8.8, |
| | | | 2.0 Hz, 1H), 4.97 (s, 2H), 4.32 (dd, J = 10.0, 5.0 Hz, 1H), 4.08 (dd, J = |
| | | | 10.4, 6.4 Hz, 1H), 3.93-3.88 (m, 5H), 3.81 (t, J = 9.2 Hz, 1H), 3.13 (s, |
| | | | 2H), 3.02 (t, J = 5.6 Hz, 2H), 2.87 (d, J = 8.8 Hz, 2H), 2.71-2.53 (m, |
| | | | 3H), 2.37-2.30 (m, 1H), 2.20-2.11 (m, 3H), 2.03 (s, 3H), 1.77 (d, J = |
| | | | 11.2 Hz, 1H), 1.68 (d, J = 12.0 Hz, 1H), 1.53-1.44 (m, 1H), 1.35-1.26 |
| | | | (m, 2H), 1.25-1.13 (m, 2H), 0.98-0.89 (m, 1H), 0.83-0.74 (m, 1H), |
| | | | 0.18-0.08 (m, 1H) |
| 125 | 223a | m/z = 1004.4 | ¹H NMR (400 MHz, DMSO-d₆) δ = 10.90 (s, 1H), 9.83 (s, 1H), 8.05- |
| | | [M + H]⁺ | 7.98 (m, 2H), 7.78 (d, J = 8.0 Hz, 1H), 7.62 (d, J = 8.0 Hz, 1H), 7.49- |
| | | | 7.41 (m, 4H), 7.40-7.32 (m, 2H), 6.92 (dd, J = 4.0, 8.0 Hz, 2H), 6.77 |
| | | | (d, J = 4.0 Hz, 1H), 6.70 (dd, J = 4.0, 8.0 Hz, 1H), 4.97 (s, 2H), 4.46- |
| | | | 4.37 (m, 1H), 4.34 (dd, J = 4.0, 8.0 Hz, 1H), 4.13 (s, 3H), 3.93-3.87 |
| | | | (m, 5H), 3.14 (s, 2H), 3.02 (t, J = 4.0 Hz, 2H), 2.93 (d, J = 8.0 Hz, 2H), |
| | | | 2.69-2.60 (m, 2H), 2.41-2.26 (m, 2H), 2.26-2.12 (m, 3H), 2.02 (s, |
| | | | 3H), 1.75 (d, J = 4.0 Hz, 2H), 1.70-1.53 (m, 2H), 1.48-1.37 (m, 1H), |
| | | | 1.27 (s, 3H), 1.24 (d, J = 8.0 Hz, 3H). |
| 126 | 223b | m/z = 1004.3 | ¹H NMR (400 MHz, DMSO-d₆) δ = 10.90 (s, 1H), 9.83 (s, 1H), 8.01 |
| | | [M + H]⁺ | (dd, J = 4.0, 8.0 Hz, 2H), 7.78 (d, J = 8.0 Hz, 1H), 7.63 (d, J = 8.0 Hz, |
| | | | 1H), 7.51-7.29 (m, 6H), 6.95 (d, J = 8.0 Hz, 1H), 6.86 (d, J = 8.8 Hz, |
| | | | 1H), 6.75 (d, J = 4.0 Hz, 1H), 6.68 (d, J = 8.0 Hz, 1H), 4.96 (s, 2H), |
| | | | 4.45-4.37 (m, 1H), 4.34 (dd, J = 4.0, 8.0 Hz, 1H), 4.13 (s, 3H), 3.92- |
| | | | 3.85 (m, 5H), 3.14 (s, 2H), 3.01 (t, J = 4.0 Hz, 2H), 2.93 (d, J = 10.0 Hz, |
| | | | 2H), 2.67-2.61 (m, 2H), 2.42-2.27 (m, 2H), 2.26-2.13 (m, 3H), 2.04 |
| | | | (s, 3H), 1.81-1.71 (m, 2H), 1.70-1.53 (m, 2H), 1.46-1.37 (m, 1H), |
| | | | 1.27 (s, 3H), 1.24 (d, J = 8.0 Hz, 3H). |
| 127 | 224a | m/z = 992.4 | ¹H NMR (400 MHz, DMSO-d₆) δ = 10.93 (s, 1H), 9.72 (s, 1H), 8.02 (d, |
| | | [M + H]⁺ | J = 7.6 Hz, 1H), 7.78 (d, J = 8.4 Hz, 1H), 7.65-7.54 (m, 2H), 7.51- |
| | | | 7.40 (m, 4H), 7.38-7.31 (m, 2H), 6.96-6.86 (m, 2H), 6.75 (s, 1H), |
| | | | 6.68 (d, J = 8.4 Hz, 1H), 4.97 (s, 2H), 4.44-4.32 (m, 2H), 4.11 (s, 3H), |
| | | | 3.90 (t, J = 5.2 Hz, 2H), 3.17-3.14 (m, 2H), 3.01 (t, J = 5.6 Hz, 2H), |
| | | | 2.93-2.86 (m, 2H), 2.71-2.58 (m, 2H), 2.42-2.31 (m, 2H), 2.18- |
| | | | 2.13 (m, 2H), 2.02 (s, 3H), 1.73-1.56 (m, 4H), 1.44-1.34 (m, 1H), |
| | | | 1.29-1.19 (m, 7H) |
| 128 | 224b | m/z = 992.4 | ¹H NMR (400 MHz, DMSO-d₆) δ = 13.04-12.37 (m, 2H), 10.93 (s, |
| | | [M + H]⁺ | 1H), 9.84-9.61 (m, 1H), 8.03 (d, J = 8.0 Hz, 1H), 7.79 (d, J = 7.6 Hz, |
| | | | 1H), 7.66-7.56 (m, 2H), 7.53-7.42 (m, 4H), 7.40-7.31 (m, 2H), 6.93 |
| | | | (dd, J = 8.8, 17.6 Hz, 2H), 6.77 (d, J = 2.0 Hz, 1H), 6.70 (dd, J = 2.0, |
| | | | 8.4 Hz, 1H), 4.97 (s, 2H), 4.47-4.32 (m, 2H), 4.11 (s, 3H), 3.95-3.87 |
| | | | (m, 2H), 3.19-3.13 (m, 1H), 3.02 (t, J = 5.6 Hz, 2H), 2.94-2.87 (m, |
| | | | 1H), 2.73-2.62 (m, 2H), 2.61-2.54 (m, 2H), 2.46-2.25 (m, 2H), 2.22- |
| | | | 2.11 (m, 2H), 2.02 (s, 3H), 1.79-1.55 (m, 4H), 1.46-1.29 (m, 3H), |
| | | | 1.28-1.19 (m, 5H) |
| 129 | 225a | m/z = 975.5 | ¹H NMR (400 MHz, DMSO-d₆) δ = 10.94 (s, 1H), 10.08 (s, 1H), 8.20 |
| | | [M + H]⁺ | (d, J = 8.8 Hz, 1H), 8.01 (t, J = 9.2 Hz, 2H), 7.78 (d, J = 7.6 Hz, 1H), |
| | | | 7.63 (d, J = 8.0 Hz, 1H), 7.49-7.41 (m, 3H), 7.39-7.32 (m, 2H), 6.98- |
| | | | 6.87 (m, 2H), 6.76 (d, J = 1.6 Hz, 1H), 6.71-6.65 (m, 1H), 4.97 (s, 2H), |
| | | | 4.46-4.29 (m, 2H), 3.96-3.86 (m, 5H), 3.19 (s, 1H), 3.16 (s, 1H), 3.02 |
| | | | (t, J = 5.6 Hz, 2H), 2.92-2.84 (m, 2H), 2.44-2.37 (m, 2H), 2.24-2.11 |
| | | | (m, 4H), 2.02 (s, 3H), 1.72-1.64 (m, 3H), 1.59-1.53 (m, 1H), 1.43- |
| | | | 1.35 (m, 1H), 1.27-1.18 (m, 7H). |
| 130 | 225b | m/z = 975.5 | ¹H NMR (400 MHz, DMSO-d₆) δ = 13.32-12.22 (m, 2H), 10.94 (s, |
| | | [M + H]⁺ | 1H), 10.15-10.02 (m, 1H), 8.21 (d, J = 8.8 Hz, 1H), 8.02 (dd, J = 8.8, |
| | | | 13.6 Hz, 2H), 7.79 (d, J = 8.0 Hz, 1H), 7.62 (d, J = 7.2 Hz, 1H), 7.50- |
| | | | 7.43 (m, 3H), 7.40-7.33 (m, 2H), 6.98-6.88 (m, 2H), 6.77 (d, J = 1.2 |
| | | | Hz, 1H), 6.73-6.67 (m, 1H), 4.97 (s, 2H), 4.45-4.31 (m, 2H), 3.96- |
| | | | 3.89 (m, 5H), 3.20 (s, 1H), 3.17 (s, 1H), 3.03 (t, J = 4.8 Hz, 2H), 2.93- |
| | | | 2.85 (m, 2H), 2.46-2.36 (m, 2H), 2.32-2.07 (m, 4H), 2.03 (s, 3H), |
| | | | 1.74-1.63 (m, 3H), 1.60-1.52 (m, 1H), 1.44-1.35 (m, 1H), 1.23 (d, J = |
| | | | 6.0 Hz, 7H). |
| 131 | 221 | m/z = 493.9 | |
| | | [M/2 + H]⁺ | |
| 132 | 211 | m/z = 982.5 | |
| | | [M + H]⁺ | |
| 133 | 174a | m/z = 486.9 | |
| | | [M/2 + H]⁺ | |
| 134 | 175a | m/z = 493.9 | |
| | | [M/2 + H]⁺ | |
| 135 | 199 | m/z = 964.5 | |
| | | [M + H]⁺ | |
| 136 | 173a | m/z = 971.3 | |
| | | [M + H]⁺ | |
| 137 | 196 | m/z = 520.9 | |
| | | [M/2 + H]⁺ | |
| 138 | 197 | m/z = 520.9 | |
| | | [M/2 + H]⁺ | |
| 139 | 177 | m/z = 501.5 | |
| | | [M/2 + H]⁺ | |
| 140 | 119 | m/z = 493.9 | |
| | | [M/2 + H]⁺ | |
| 141 | 109 | m/z = 489.9 | |
| | | [M/2 + H]⁺ | |
| 142 | 201 | m/z = 994.5 | |
| | | [M + H]⁺ | |
| 143 | 127 | m/z = 1002.4 | |
| | | [M + H]⁺ | |
| 144 | 148 | m/z = 1027.4 | |
| | | [M + H]⁺ | |
| 145 | 123 | m/z = 495.9 | |
| | | [M/2 + H]⁺ | |
| 146 | 117 | m/z = 493.9 | |
| | | [M/2 + H]⁺ | |
| 147 | 186 | m/z = 522.9 | |
| | | [M/2 + H]⁺ | |
| 148 | 183 | m/z = 1032.4 | |
| | | [M + H]⁺ | |
| 149 | 198 | m/z = 964.5 | |
| | | [M + H]⁺ | |
| 150 | 101 | m/z = 961.3 | |
| | | [M + H]⁺ | |
| 151 | 121 | m/z = 495.9 | |
| | | [M/2 + H]⁺ | |
| 152 | 185 | m/z = 522.9 | |
| | | [M/2 + H]⁺ | |
| 153 | 147 | m/z = 1027.5 | |
| | | [M + H]⁺ | |
| 154 | 182 | m/z = 516.8 | |
| | | [M/2 + H]⁺ | |
| 155 | 176a | m/z = 972.5 | |
| | | [M + H]⁺ | |
| 156 | 146 | m/z = 1015.4 | |
| | | [M + H]⁺ | |
| 157 | 120 | m/z = 990.5 | |
| | | [M + H]⁺ | |
| 158 | 125 | m/z = 493.5 | |
| | | [M/2 + H]⁺ | |
| 159 | 136 | m/z = 520.9 | |
| | | [M/2 + H]⁺ | |
| 160 | 145 | m/z = 1015.4 | |
| | | [M + H]⁺ | |
| 161 | 137 | m/z = 1040.5 | |
| | | [M + H]⁺ | |
| 162 | 200 | m/z = 994.5 | |
| | | [M + H]⁺ | |
| 163 | 118 | m/z = 986.6 | |
| | | [M + H]⁺ | |
| 164 | 126 | m/z = 501.9 | |
| | | [M/2 + H]⁺ | |
| 165 | 139 | m/z = 1040.5 | |
| | | [M + H]⁺ | |
| 166 | 215d | m/z = 1028.4 | ¹H NMR (400 MHz, DMSO-d₆) δ 12.96-12.75 (m, 1H), 12.69-12.24 |
| | | [M + H]⁺ | (m, 1H), 10.88 (s, 1H), 9.89-9.71 (m, 1H), 8.03 (t, J = 3.6 Hz, 2H), |
| | | | 7.79 (d, J = 8.0 Hz, 1H), 7.66-7.59 (m, 2H), 7.51-7.43 (m, 3H), 7.42- |
| | | | 7.33 (m, 3H), 7.24-7.17 (m, 2H), 6.99 (d, J = 8.8 Hz, 1H), 6.67 (d, J = |
| | | | 7.6 Hz, 1H), 5.06-4.93 (m, 2H), 4.32 (dd, J = 5.2, 9.6 Hz, 1H), 4.13- |
| | | | 4.05 (m, 2H), 3.95-3.90 (m, 5H), 3.03 (t, J = 5.6 Hz, 3H), 2.96-2.88 |
| | | | (m, 1H), 2.68-2.59 (m, 2H), 2.46-2.28 (m, 4H), 2.21-2.14 (m, 1H), |
| | | | 1.80-1.72 (m, 2H), 1.68-1.61 (m, 2H), 1.38 (s, 3H), 1.32-1.13 (m, |
| | | | 2H), 1.05 (d, J = 5.2 Hz, 3H). |
| 167 | 2001a | m/z = 953.5 | |
| | | [M + H]⁺ | |
| 168 | 2002 | m/z = 931.2 | |
| | | [M + H]⁺ | |
| 169 | 2003 | m/z = 903.3 | |
| | | [M + H]⁺ | |

### Example B1

### HiBiT Based Degradation Assay

Human BCL-X_{L} protein coding open reading frame fused with N-terminal HiBiT coding sequence (N-HiBiT-BCL-X_{L}) was synthesized from Integrated DNA Technologies (IDT). Next, N-HiBiT-BCL-X_{L} sequence was cloned into pLV-UBC-PGK-Puro, a lentivirus plasmid purchased from Vectorbuilder, to generate pLV-UBC-N-HiBiT-BCL-X_{L}-PGK-puro. Lentiviral particles were generated from Lenti-X™ 293T cells (Clontech) by co-transfection of pLV-UBC-N-HiBiT-BCL-X_{L}-PGK-puro plasmids and lentiviral packaging plasmid mix (Cellecta). HT-1080 [HT1080](ATCC CCL-121™) cells were infected with the lentivirus, and HT1080 cells stably integrated with the lentiviral vector were established by incubation with 1 μg/mL puromycin (Thermofisher). HT1080 cells expressing N-terminal HiBiT tagged BCL-X_{L} were dispensed into 384-well plates pre-spotted with compounds at varying concentrations. Three thousand cells were seeded into each well in 40 μL of RPMI 1640 media. After 24 hours of incubation at 37° C. with 5% CO₂, 30 μL of the NANO-GLO® HiBiT Lytic Detection System working solution (Promega) was added to each well and incubated at room temperature for 30 min. After incubation, luminescence was read on a PHERAstar FSX Plate Reader (BMG). BCL-X_{L} degradation at each indicated concentration was normalized with DMSO control. The BCL-X_{L} degradation curves were plotted using a four-parameter logistic model. For compounds displaying hook effects, curve fitting was performed using bottom point of hook curves (i.e., the point of Yₘᵢₙ) as bottom plateau to calculate EC₅₀. For compounds with multiple data points, the geometric mean is presented. Yₘᵢₙ and EC₅₀ values for certain compounds are shown in Table B1.

TABLE B1

| Compound No. | EC₅₀ (24 h) | Yₘᵢₙ (24 h) |
|---|---|---|
| 101 | +++ | C |
| 103 | ++ | B |
| 104 | +++ | B |
| 105 | +++ | A |
| 106 | +++ | B |
| 107 | ++++ | A |
| 108 | +++ | B |
| 109 | +++ | C |
| 112 | ++++ | A |
| 113 | ++++ | B |
| 114 | +++ | B |
| 115 | +++ | B |
| 116 | ++++ | B |
| 117 | +++ | B |
| 118 | ++ | B |
| 119 | ++++ | B |
| 120 | +++ | B |
| 121 | +++ | B |
| 122 | +++ | B |
| 123 | +++ | B |
| 124 | ++++ | B |
| 125 | +++ | B |
| 126 | ++ | C |
| 127 | +++ | A |
| 128 | ++++ | A |
| 129 | ++++ | B |
| 130a | +++ | B |
| 130b | +++ | B |
| 131 | ++++ | B |
| 132 | +++ | B |
| 133a | +++ | B |
| 133b | +++ | A |
| 134 | +++ | B |
| 135 | ++++ | A |
| 136 | +++ | B |
| 137 | +++ | C |
| 138a | ++++ | B |
| 138b | +++ | B |
| 139 | + | C |
| 140a | ++++ | A |
| 140b | +++ | A |
| 141 | ++++ | A |
| 142 | +++ | A |
| 143 | +++ | B |
| 144 | +++ | B |
| 145 | +++ | C |
| 146 | +++ | B |
| 147 | +++ | B |
| 148 | +++ | B |
| 149 | ++++ | A |
| 150 | ++++ | B |
| 151 | ++++ | A |
| 152 | ++++ | B |
| 153 | ++++ | A |
| 154 | ++++ | B |
| 155 | ++++ | A |
| 156 | ++++ | B |
| 157 | +++ | A |
| 158 | +++ | B |
| 159 | +++ | B |
| 160 | ++++ | B |
| 161 | +++ | B |
| 162 | +++ | B |
| 163 | ++++ | B |
| 164 | +++ | B |
| 165 | ++++ | B |
| 166 | +++ | B |
| 167 | ++++ | A |
| 168 | ++++ | B |
| 169 | ++++ | B |
| 170 | ++++ | A |
| 171 | +++ | B |
| 172 | +++ | B |
| 173a | ++++ | B |
| 174a | ++++ | A |
| 175a | ++++ | B |
| 176a | +++ | C |
| 177 | ++++ | B |
| 178 | ++++ | A |
| 179 | ++++ | A |
| 180 | +++ | B |
| 181 | +++ | B |
| 182 | +++ | B |
| 183 | +++ | B |
| 184 | ++++ | B |
| 185 | +++ | B |
| 186 | +++ | B |
| 187 | ++++ | A |
| 188 | ++++ | B |
| 189 | ++++ | B |
| 190 | ++++ | B |
| 191 | ++++ | B |
| 192 | ++++ | B |
| 193 | +++ | B |
| 194 | ++++ | B |
| 195 | ++++ | B |
| 196 | ++++ | B |
| 197 | ++++ | B |
| 198 | +++ | B |
| 199 | ++++ | A |
| 200 | +++ | B |
| 201 | +++ | A |
| 202 | ++++ | B |
| 203 | +++ | B |
| 204 | ++++ | B |
| 205 | ++++ | A |
| 206 | +++ | B |
| 207 | ++++ | B |
| 208a | +++ | B |
| 208b | +++ | B |
| 208c | +++ | B |
| 208d | +++ | B |
| 209a | +++ | A |
| 209b | +++ | B |
| 209c | ++++ | A |
| 209d | ++++ | B |
| 210a | +++ | A |
| 210b | +++ | A |
| 210c | ++++ | A |
| 210d | +++ | A |
| 211 | +++ | C |
| 212 | +++ | B |
| 213 | ++++ | A |
| 214 | ++++ | B |
| 215a | +++ | B |
| 215b | +++ | B |
| 215c | +++ | B |
| 215d | +++ | B |
| 216a | +++ | B |
| 216b | ++++ | B |
| 216c | +++ | B |
| 216d | +++ | B |
| 217 | ++++ | B |
| 218 | ++++ | A |
| 219a | ++++ | A |
| 219b | ++++ | A |
| 219c | ++++ | A |
| 219d | ++++ | A |
| 220 | +++ | C |
| 221 | ++++ | A |
| 222 | ++++ | A |
| 223a | +++ | B |
| 223b | +++ | A |
| 224a | +++ | B |
| 224b | +++ | A |
| 225a | +++ | B |
| 225b | +++ | B |
| 1001a | +++ | B |
| 1001b | +++ | B |
| 1001c | +++ | B |
| 1001d | +++ | B |
| 1002a | ++++ | B |
| 1002b | ++++ | B |
| 1002c | ++++ | A |
| 1002d | ++++ | A |
| 1003a | ++++ | A |
| 1003b | ++++ | B |
| 1003c | ++++ | A |
| 1003d | ++++ | A |
| 1004a | +++ | B |
| 1004b | +++ | B |
| 1004c | ++++ | B |
| 1004d | +++ | B |
| 2001a | ++++ | B |
| 2002 | +++ | C |
| 2003 | +++ | C |

Notes:
(1) “A”: Yₘᵢₙ < 30%; “B”: 30% ≤ Yₘᵢₙ < 50%; “C”: 50% ≤ Yₘᵢₙ < 70%; “D”: Yₘᵢₙ ≥ 70%
(2) “++++”: EC₅₀ < 10 nM; “+++”: 10 nM ≤ EC₅₀ < 100 nM; “++”: 100 nM ≤ EC₅₀ < 1000 nM; “+”: 1000 nM ≤ EC₅₀ < 10,000 nM

### Example B2

### Western Blotting Based Degradation Assay

BCL-X_{L} degradation in human cancer cell lines is measured using traditional Western blotting methods as well as with the automated “Jess” instrument from ProteinSimple. Cells are seeded at 0.5-1 million cells per well in 2 mL of growth media (RPMI+10% FBS) in 6-well plates. The plates are treated with compounds at varying concentrations for up to 5 days. Cells are harvested and lysed using RIPA Buffer (#9806S, Cell Signaling Technology) with Halt Protease & Phosphatase Inhibitor Cocktail (#78441, Thermo Fisher). Protein concentrations of each sample are quantified using Pierce™ BCA Protein Assay Kit (#23225, Thermo Fisher). For the Jess instrument the standard protocol provided by the manufacturer using the Jess Separation Module SM-W004 is followed. For traditional Western blotting, samples resolved on a 4-20% Criterion TGX gel (#5671094, BioRad) and transferred to a PVDF membrane using the Trans-Blot Turbo Transfer system (#1704157, BioRad). Chemiluminescent detection of BCL-X_{L} is performed using a BCL-X_{L} antibody (#2764, Cell Signaling Technology) according to the manufacturer's instructions.

### Example B3

### MOLT-4 Proliferation Assay

MOLT-4 (ATCC CRL-1582™) cells are seeded at 4,000 cells per well in 50 L RPMI 1640 media in 384-well plates. The plates are pre-spotted with compounds at varying concentrations. After 3 days of incubation at 37° C. with 5% CO₂, cell viability is assessed using CELLTITER-GLO@Cell Viability Assay kit according to manufacturer's instructions (Promega). Relative cell proliferation at each concentration is normalized against DMSO control.

### Example B4

### Platelet Viability Assay

Human platelet-rich plasma (PRP, SER-PRP-SDS) is obtained from Zenbio. Sodium citrate is used as anticoagulant. To remove any contaminating blood cells, the samples are spun at 200×g at room temperature for 20 minutes in 50 mL falcon tubes, and the supernatant is transferred to a new tube. One hundred microliter PRP are added to each well of a 96-well plate spotted with compounds at varying concentrations. Plates are incubated at 37° C. with 5% CO₂ for 24 hours, and platelet viability are assessed with CELLTITERGLO@Cell Viability Assay kit according to manufacturer's instructions (Promega). Relative viability at each concentration is normalized against DMSO control.

### EMBODIMENTS

### Exemplary P01 Embodiments

Embodiment 1. A compound of Formula (I)

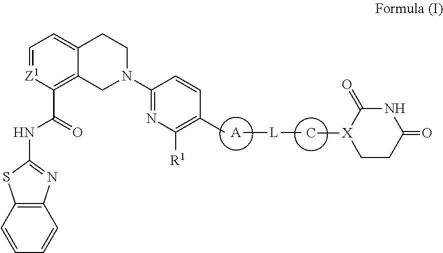

or a pharmaceutically acceptable salt thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{a};
Ring A is selected from the group consisting of (A1) and (A2):

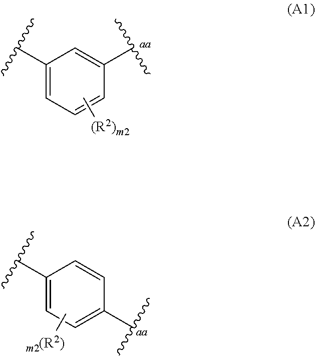

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to L;
L is -(L^{A})ₙ₁-, wherein:
n1 is an integer from 4 to 10,
one L^{A} is L^{A4};
0-3 L^{A} are independently selected L^{A3}, and
each remaining L^{A} is an independently selected L^{A1};
each L^{A1} is independently selected from the group consisting of: —CH₂—, —CHR^{L}—, and —C(R^{L})₂—, wherein: each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
each L^{A3} is independently selected from the group consisting of: —N(R^{d})—, —N(R^{b})—, —O—, —S(O)₀₋₂—, and C(═O);
L^{A4} is independently selected from the group consisting of:
(a) C₃₋₁₂ cycloalkylene or 4-15 membered heterocyclylene, each of which is optionally substituted with 1-3 R^{L4}; and
(b) phenylene or 5-6 membered heteroarylene, each of which is optionally substituted with 1-3 R^{L4};
wherein each R^{L4} is independently selected from the group consisting of R^{a} and R^{b};
Ring C is selected from the group consisting of:

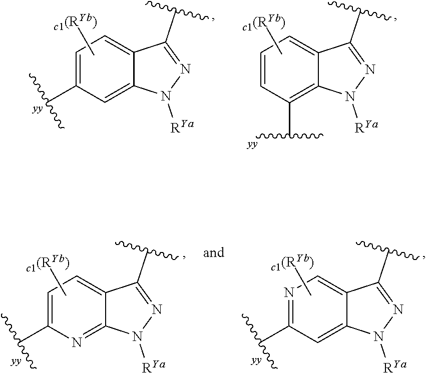

wherein:
R^{Ya} is selected from the group consisting of: H, R^{b}, and C₁₋₆ alkyl optionally substituted with 1-3 R^{c};
each R^{Yb} is independently selected from the group consisting of R^{a} and R^{b};
c1 is 0, 1, or 2; and
yy represents the point of attachment to L;
X is CH or N;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(1) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁-6 alkyl), S(O)₁₋₂(C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h},
each R^{g} is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂,
provided that the compound of Formula (I) does not contain any 0-0, N—O, N—N, N—S(O)₀, O—S(O)₀₋₂, N-halo, O-halo, or S(O)₀₋₂-halo bonds.
Embodiment 2. The compound of Embodiment 1, wherein the compound of Formula (I) is other than a compound selected from the group consisting of the compounds depicted in Table R1, or a pharmaceutically acceptable salt thereof.
Embodiment 3. The compound of Embodiment 1 or 2, wherein Z¹ is N.
Embodiment 4. The compound of Embodiment 1 or 2, wherein Z¹ is CH.
Embodiment 5. The compound of any one of Embodiments 1-4, wherein Ring A is (A1).
Embodiment 6. The compound of any one of Embodiments 1-4, wherein Ring A is (A2).
Embodiment 7. The compound of any one of Embodiments 1-6, wherein m2 is 1.
Embodiment 8. The compound of any one of Embodiments 1-5 or 7, wherein Ring A is

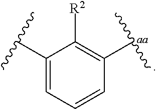

Embodiment 9. The compound of any one of Embodiments 1-4 or 6-7, wherein Ring A is

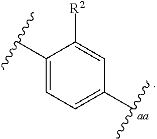

Embodiment 10. The compound of any one of Embodiments 1-9, wherein R² is C₁₋₃ alkyl optionally substituted with 1-3 R^{c} (e.g., C₁₋₃ alkyl optionally substituted with 1-3 F).
Embodiment 11. The compound of any one of Embodiments 1-10, wherein R² is CH₃.
Embodiment 12. The compound of any one of Embodiments 1-10, wherein R² is CF₃.
Embodiment 13. The compound of any one of Embodiments 1-5 or 7-8, wherein Ring A is:

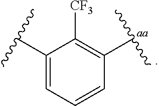 [SMILES: *c1cccc(*)c1C(F)(F)F]

Embodiment 14. The compound of any one of Embodiments 1-5 or 7-8, wherein Ring A is

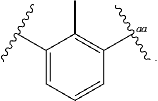 [SMILES: *c1cccc(*)c1C]

Embodiment 15. The compound of any one of Embodiments 1-4, 6-7, or 9, wherein Ring A is:

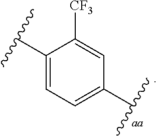 [SMILES: *c1ccc(*)c(C(F)(F)F)c1]

Embodiment 16. The compound of any one of Embodiments 1-4, 6-7, or 9, wherein Ring A is:

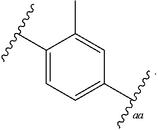 [SMILES: *c1ccc(*)c(C)c1]

Embodiment 17. The compound of any one of Embodiments 1-16, wherein n1 is an integer from 5 to 9 (e.g., 5, 6, 7, or 8).
Embodiment 18. The compound of any one of Embodiments 1-17, wherein n1 is an integer from 5 to 9; and L is:

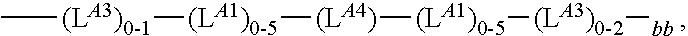

wherein bb represents the point of attachment to Ring C.
Embodiment 19. The compound of any one of Embodiments 1-18, wherein n1 is an integer from 5 to 9; and L is selected from the group consisting of:

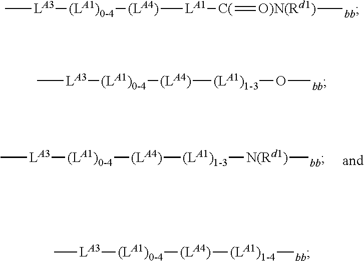

wherein bb represents the point of attachment to Ring C; and R^{d1} is H or C₁₋₃ alkyl.
Embodiment 20. The compound of any one of Embodiments 1-19, wherein n1 is an integer from 5 to 9; and L is:

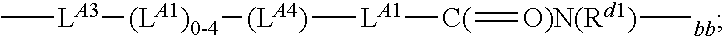

wherein bb represents the point of attachment to Ring C; and R^{d1} is H or C₁₋₃ alkyl.
Embodiment 21. The compound of any one of Embodiments 1-20, wherein n1 is an integer from 5 to 9; and L is:

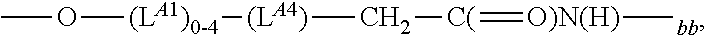

wherein bb represents the point of attachment to Ring C.
Embodiment 22. The compound of any one of Embodiments 1-21, wherein L^{A4} is a 5-12 membered nitrogen-containing heterocyclylene optionally substituted with 1-3 R^{L4}
Embodiment 23. The compound of any one of Embodiments 1-22, wherein each R^{L4} is independently selected from the group consisting of: —F, CN, OH, C₁₋₃ alkoxy, and C₁₋₃ alkyl optionally substituted with 1-3 F.
Embodiment 24. The compound of any one of Embodiments 1-21, wherein L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 25. The compound of any one of Embodiments 1-21 or 24, wherein L^{A4} is

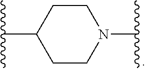 [SMILES: *C1CCN(*)CC1]

Embodiment 26. The compound of any one of Embodiments 1-21 or 24, wherein L^{A4} is

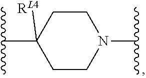

wherein R^{L4} is —F or —OH (e.g., —OH).
Embodiment 27. The compound of any one of Embodiments 1-21, wherein L^{A4} is a bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 28. The compound of any one of Embodiments 1-21 or 27, wherein L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 29. The compound of any one of Embodiments 1-21 or 27-28, wherein L^{A4} is selected from the group consisting of:

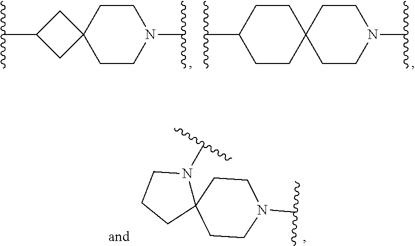

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 30. The compound of any one of Embodiments 1-21 or 27-29, wherein L^{A4} is

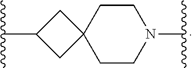 [SMILES: *C1CC2(CCN(*)CC2)C1]

Embodiment 31. The compound of any one of Embodiments 1-21 or 27, wherein L^{A4} is a bridged or fused bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 32. The compound of any one of Embodiments 1-21, 27, or 31, wherein L^{A4} is selected from the group consisting of:

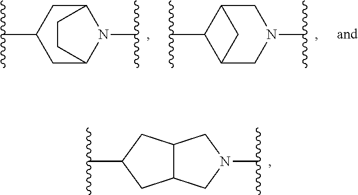

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 33. The compound of any one of Embodiments 1-32, wherein each L^{A}1 is CH₂.
Embodiment 34. The compound of any one of Embodiments 1-32, wherein one L^{A1} is CHR^{L} or C(R^{L})₂, wherein each R^{L} is independently —F or C₁₋₃ alkyl optionally substituted with 1-3 F; and each remaining L^{A1} is CH₂.
Embodiment 35. The compound of any one of Embodiments 1-34, wherein Ring C is

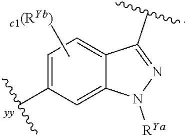

Embodiment 36. The compound of any one of Embodiments 1-35, wherein Ring C is

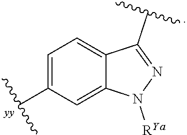

Embodiment 37. The compound of any one of Embodiments 1-35, wherein Ring C is

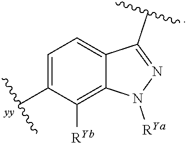

Embodiment 38. The compound of any one of Embodiments 1-35, wherein Ring C is

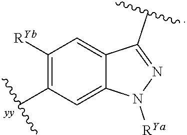

Embodiment 39. The compound of any one of Embodiments 1-38, wherein R^{Ya} is C₁₋₃ alkyl optionally substituted with 1-3 R^{c} (e.g., C₁₋₃ alkyl optionally substituted with 1-3 F).
Embodiment 40. The compound of any one of Embodiments 1-39, wherein R^{Ya} is methyl.
Embodiment 41. The compound of any one of Embodiments 1-35 or 37-40, wherein each R^{Yb} is independently selected from the group consisting of: —F, C₁₋₃ alkoxy, and C₁₋₃ alkyl optionally substituted with 1-3 F.
Embodiment 42. The compound of any one of Embodiments 1-35 or 37-41, wherein each R^{Yb} is independently selected from the group consisting of: —F, OMe, CH₃, CHF₂, CH₂F, and CF₃.
Embodiment 43. The compound of any one of Embodiments 1-35 or 37-41, wherein at least one R^{Yb} is C₁₋₃ alkoxy (e.g., OMe).
Embodiment 44. The compound of any one of Embodiments 1-34, wherein Ring C is

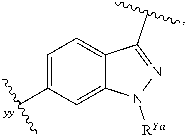

wherein R^{Ya} is C₁₋₃ alkyl optionally substituted with 1-3 R^{c} (e.g., C₁₋₃ alkyl optionally substituted with 1-3 F).
Embodiment 45. The compound of any one of Embodiments 1-34, wherein Ring C is

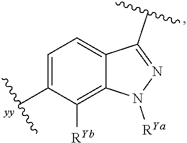

wherein R^{Ya} is C₁₋₃ alkyl optionally substituted with 1-3 R^{c} (e.g., C₁₋₃ alkyl optionally substituted with 1-3 F); and
R^{Yb} is independently selected from the group consisting of: —F, C₁₋₃ alkoxy, and C₁₋₃ alkyl optionally substituted with 1-3 F.
Embodiment 46. The compound of Embodiment 45, wherein R^{Yb} is C₁₋₃ alkoxy (e.g., methoxy).
Embodiment 47. The compound of Embodiment 1 or 18, wherein the compound is a compound of Formula (I-1) or (I-2):

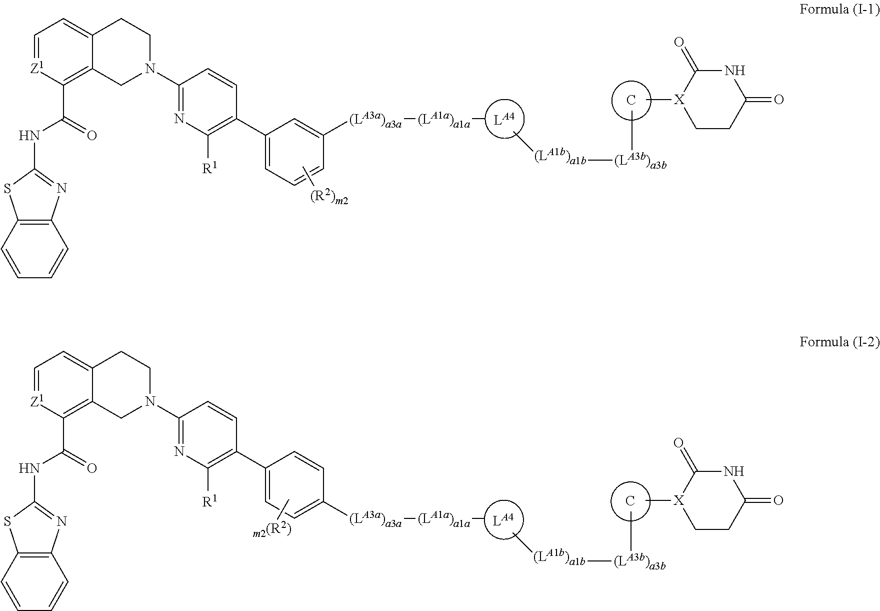

or a pharmaceutically acceptable salt thereof, wherein:
m2 is 0 or 1;
each L^{A3a} and L^{A3b} is an independently selected L^{A3};
a3a is 0 or 1;
a3b is 0, 1, or 2;
each L^{A1a} and L^{A1}b is an independently selected L^{A1};
a1a and a1b are independently integers from 0 to 4;
provided that: a3a+a1a+a1b+a3b is from 4 to 8; and
L^{A4} is a 5-12 membered nitrogen-containing heterocyclylene optionally substituted with 1-3 R^{L4};
Embodiment 48. The compound of Embodiment 47, wherein the compound is a compound of Formula (I-1), or a pharmaceutically acceptable salt thereof; and one or more of (1)-(3) applies:
(1) Z¹ is N;
(2) Ring C is

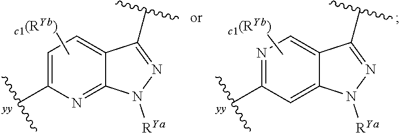

and/or
(3) c1 is 1 or 2, wherein at least one R^{Yb} is C₁₋₃ alkoxy (e.g., OMe).
Embodiment 49. The compound of Embodiment 47, wherein the compound is a compound of Formula (I-2), or a pharmaceutically acceptable salt thereof; and one or more of (1)-(4) applies:
(1) Z¹ is N;
(2) Ring C is

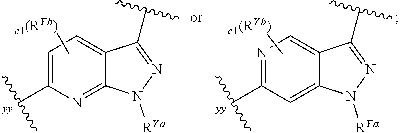

(3) c1 is 1 or 2, wherein at least one R^{Yb} is C₁₋₃ alkoxy; and/or
(4) m2 is 1; and R² is CF₃.
Embodiment 50. The compound of Embodiment 47, wherein the compound is a compound of Formula (I-a1) or (I-a2):

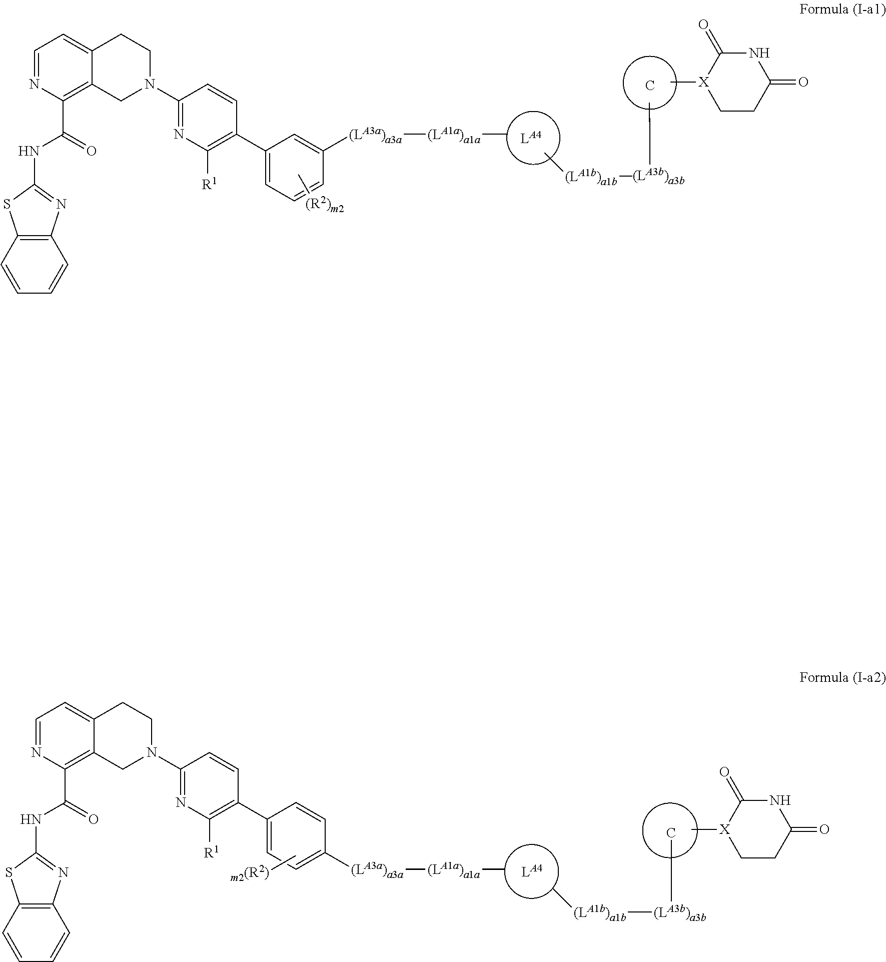

or a pharmaceutically acceptable salt thereof.
Embodiment 51. The compound of Embodiment 47, wherein the compound is a compound of Formula (I-b1) or (I-b2):

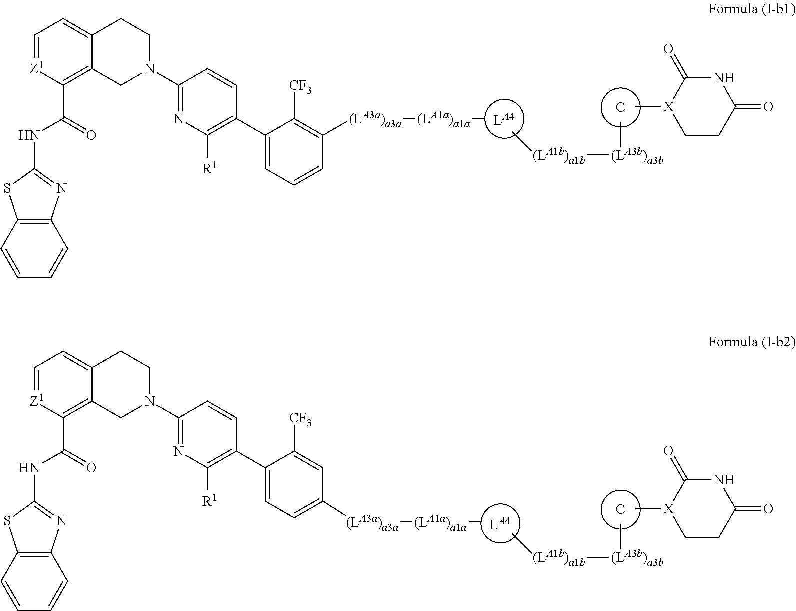

or a pharmaceutically acceptable salt thereof.
Embodiment 52. The compound of Embodiment 51, wherein the compound is other than Compound Nos. R126 or R313, as depicted in Table R1, or a pharmaceutically acceptable salt thereof.
Embodiment 53. The compound of Embodiment 51, wherein the compound is a compound of Formula (I-b2), or a pharmaceutically acceptable salt thereof.
Embodiment 54. The compound of Embodiment 51, wherein the compound is a compound of Formula (I-b1), or a pharmaceutically acceptable salt thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and/or
(5) a1a is 0 or 1.
Embodiment 55. The compound of Embodiment 47, wherein the compound is a compound of Formula (I-c1) or (I-c2):

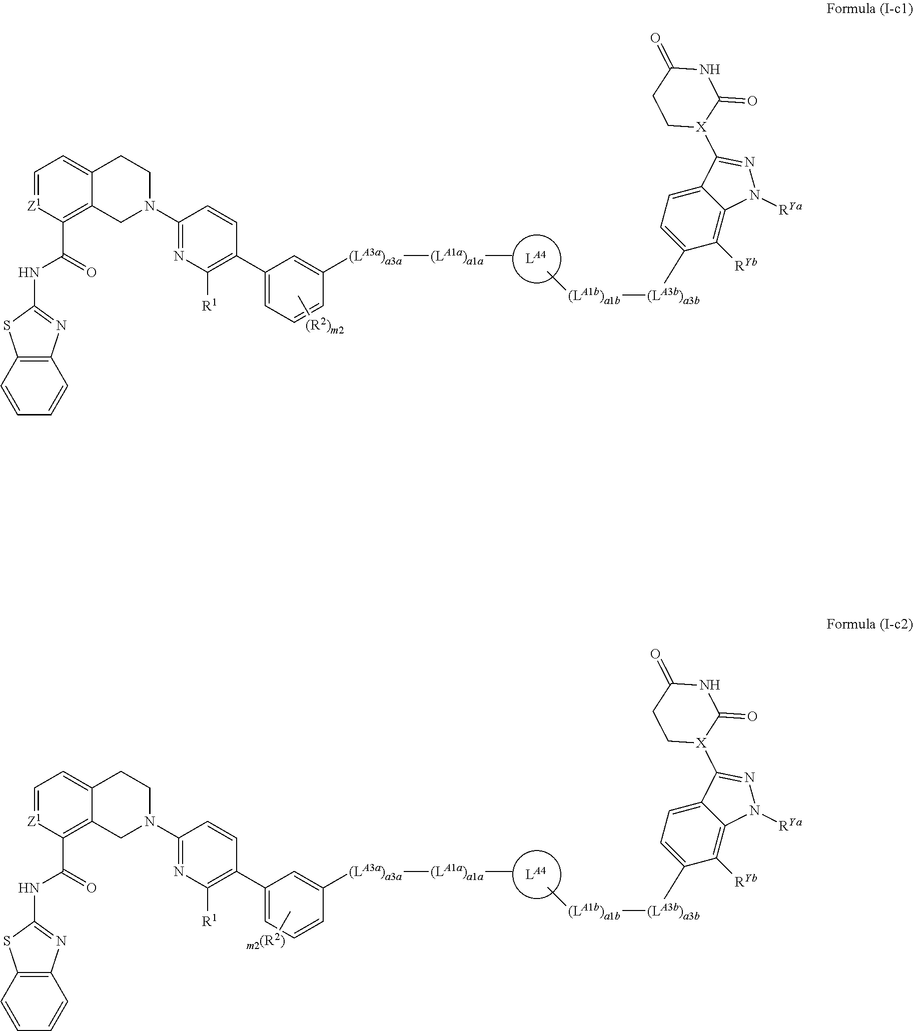

or a pharmaceutically acceptable salt thereof, wherein:
R^{Yb} is F, C₁₋₃ alkoxy, or C₁₋₃ alkyl optionally substituted with 1-3 —F.
Embodiment 56. The compound of Embodiment 55, wherein the compound is other than Compound Nos. R302 or R356, as depicted in Table R1, or a pharmaceutically acceptable salt thereof.
Embodiment 57. The compound of Embodiment 55, wherein R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4}.
(4) at least one L^{A1a} is selected from the group consisting of CHR^{L} and C(R^{L})₂; and/or
(5) a1a is 0, 1, or 2.
Embodiment 58. The compound of Embodiment 55, wherein R^{Yb} is C₁₋₃ alkoxy (e.g., OMe).
Embodiment 59. The compound of Embodiment 47, wherein the compound is a compound of Formula (I-d1) or (I-d2):

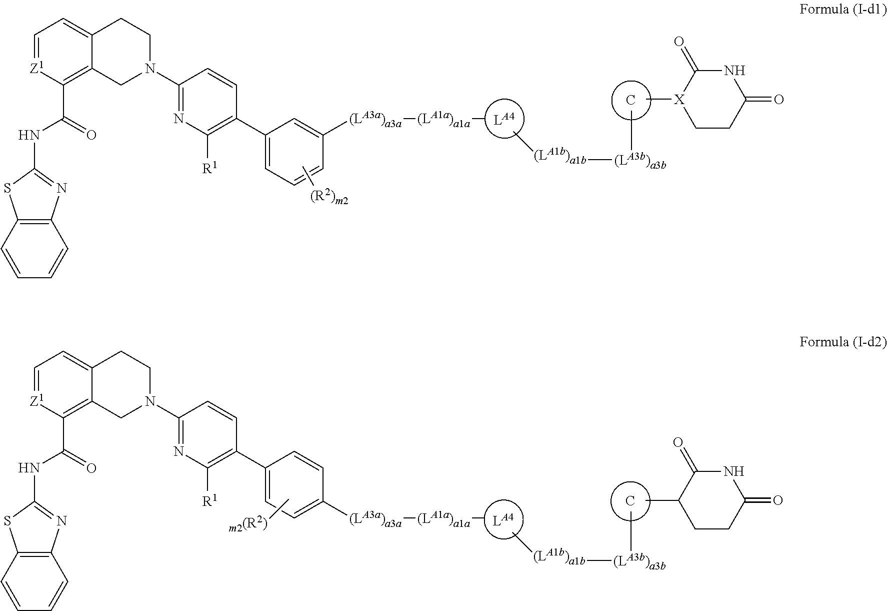

or a pharmaceutically acceptable salt thereof, wherein:
L^{A4} is

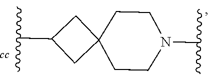

wherein cc represents point of attachment to -(L^{A1a})ₐ₁ₐ-.
Embodiment 60. The compound of Embodiment 59, wherein the compound is other than Compound Nos. R156 or R226, as depicted in Table R1, or a pharmaceutically acceptable salt thereof.
Embodiment 61. The compound of Embodiment 59 wherein the compound is a compound of Formula (I-d1), or a pharmaceutically acceptable salt thereof; and one of more of (1)-(5) applies:
(1) m2 is 1, and R² is CF₃;
(2) a1a is 0;
(3) a1a is 2 or 3;
(4) c1 is 1 or 2; and/or
(5) at least one L^{A1a} is selected from the group consisting of CHR^{L} and C(R^{L})₂.
Embodiment 62. The compound of Embodiment 59, wherein the compound is a compound of Formula (I-d2), or a pharmaceutically acceptable salt thereof.
Embodiment 63. The compound of any one of Embodiments 47-62, wherein alb is 1; and L^{A1}b is —CH₂—.
Embodiment 64. The compound of any one of Embodiments 47-63, wherein a3a is 1; L^{A3a} is —O—; a3b is 2; and -(L^{A3}b)_{a3b}- is —C(═O)NH— that is attached to Ring C at the nitrogen atom.
Embodiment 65. The compound of any one of Embodiments 47 or 63-64, wherein the compound is a compound of Formula (I-e1) or (I-e2):

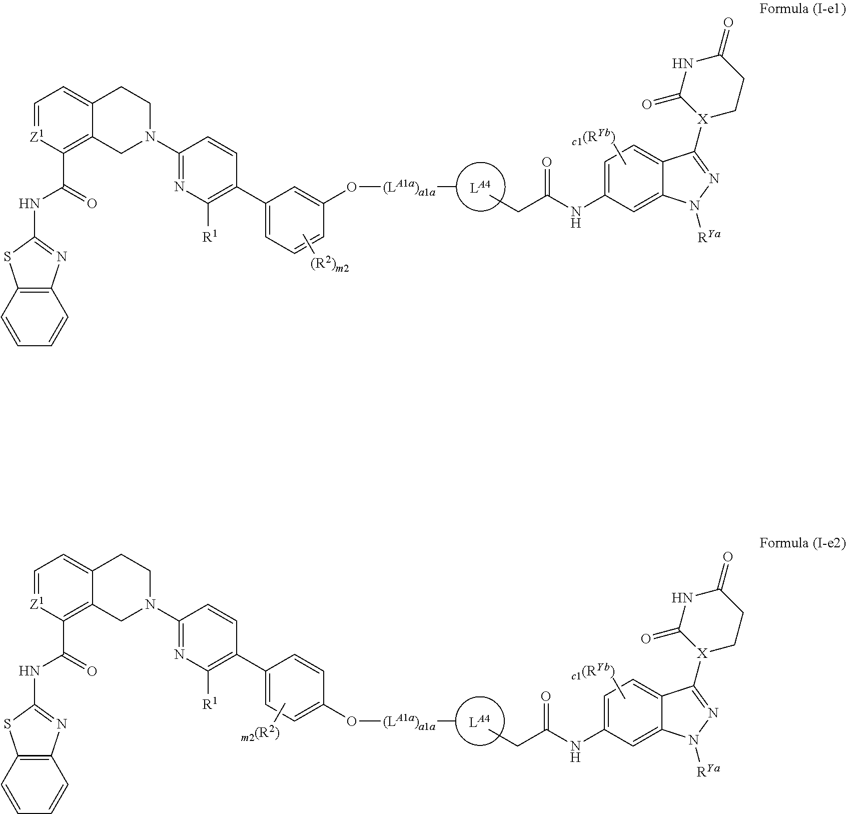

or a pharmaceutically acceptable salt thereof, wherein:
a1a is an integer from 0 to 4;
m2 is 0 or 1; and
R² is CH₃ or CF₃.
Embodiment 66. The compound of Embodiment 65, wherein the compound is a compound of Formula (I-e1), or a pharmaceutically acceptable salt thereof; and one or more of (1)-(3) applies:
(1) Z¹ is N;
(2) Ring C is

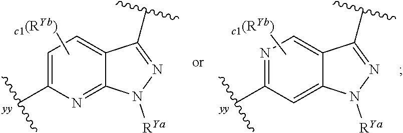

and/or
(3) c1 is 1 or 2, wherein at least one R^{Yb} is C₁₋₃ alkoxy.
Embodiment 67. The compound of Embodiment 65, wherein the compound is a compound of Formula (I-e2), or a pharmaceutically acceptable salt thereof; and one or more of (1)-(4) applies:
(1) Z¹ is N;
(2) Ring C is

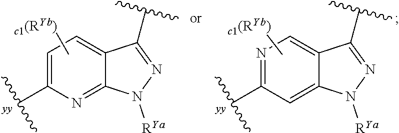

(3) c1 is 1 or 2, wherein at least one R^{Yb} is C₁₋₃ alkoxy; and/or
(4) m2 is 1; and R² is CF₃.
Embodiment 68. The compound of any one of Embodiments 47, 50, or 63-64, wherein the compound is a compound of Formula (I-f1) or (I-f2):

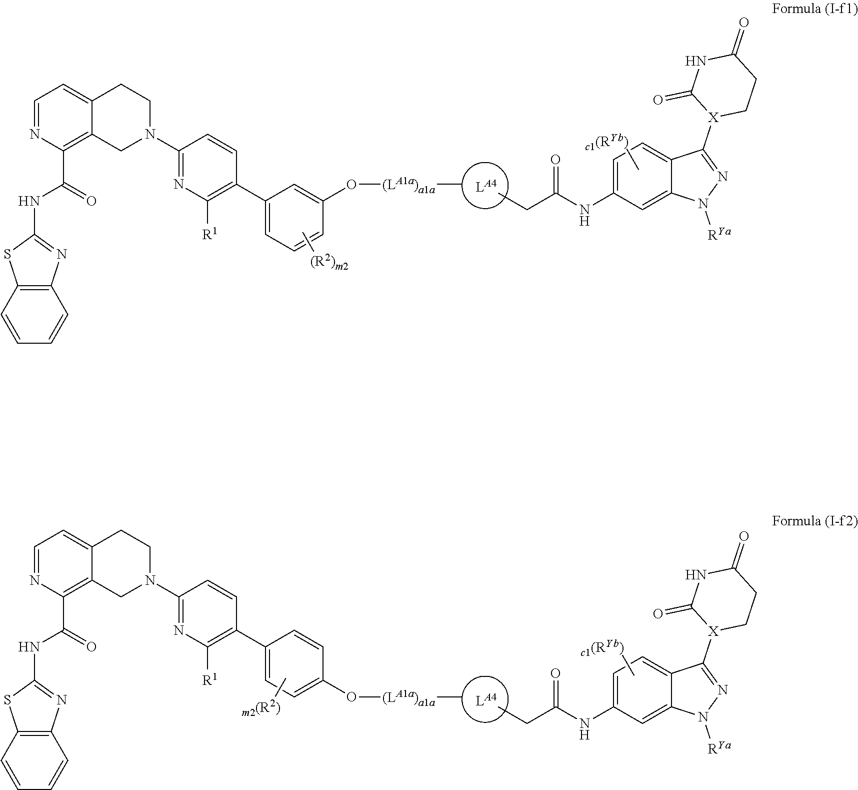

or a pharmaceutically acceptable salt thereof, wherein:
a1a is an integer from 0 to 4;
m2 is 0 or 1; and
R² is CH₃ or CF₃.
Embodiment 69. The compound of any one of Embodiments 47, 51, or 63-64, wherein the compound is a compound of Formula (I-g1) or (I-g2):

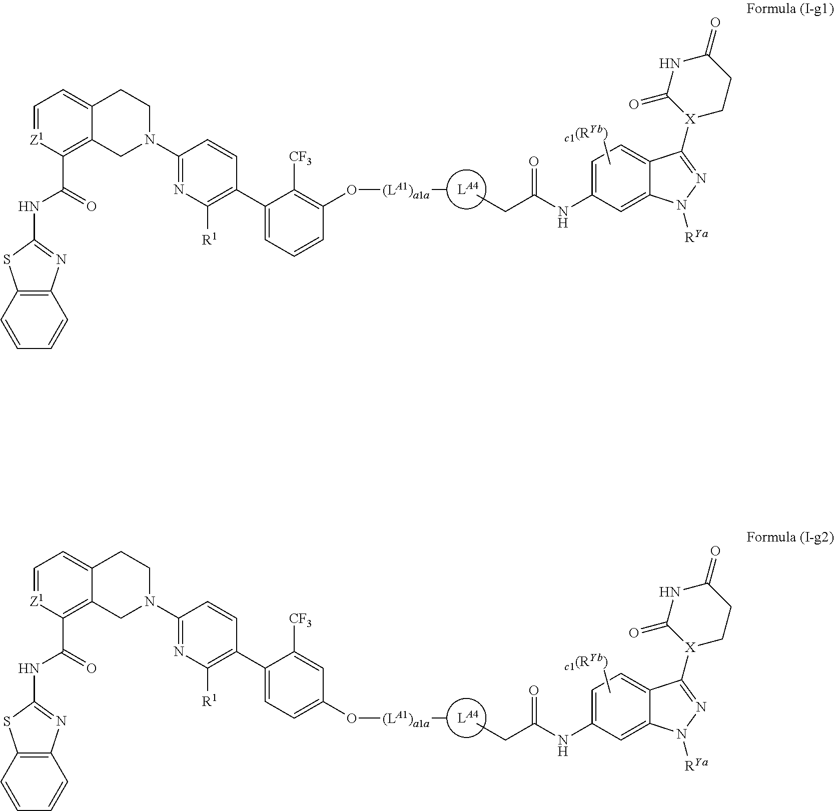

or a pharmaceutically acceptable salt thereof, wherein:
a1a is an integer from 0 to 4;
m2 is 0 or 1; and
R² is CH₃ or CF₃.
Embodiment 70. The compound of Embodiment 69, wherein the compound is other than Compound Nos. R126 or R313, as depicted in Table R1, or a pharmaceutically acceptable salt thereof.
Embodiment 71. The compound of Embodiment 69, wherein the compound is a compound of Formula (I-g2), or a pharmaceutically acceptable salt thereof.
Embodiment 72. The compound of Embodiment 69, wherein the compound is a compound of Formula (I-g1), or a pharmaceutically acceptable salt thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and/or
(5) a1a is 0 or 1.
Embodiment 73. The compound of any one of Embodiments 47, 55, or 63-64, wherein the compound is a compound of Formula (I-h1) or (I-h2):

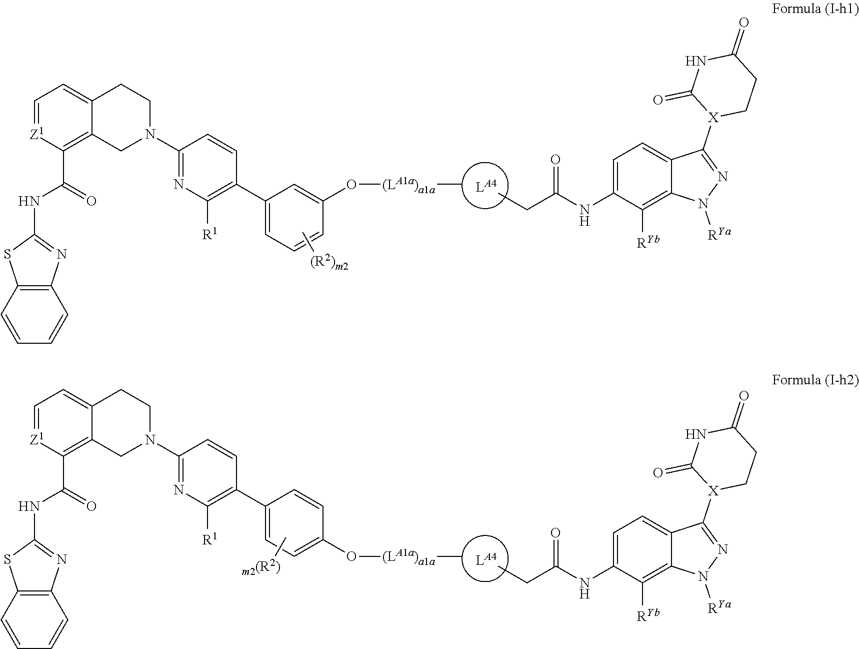

or a pharmaceutically acceptable salt thereof, wherein:
a1a is an integer from 0 to 4;
m2 is 0 or 1;
R² is CH₃ or CF₃; and
R^{Yb} is F, C₁₋₃ alkoxy, or C₁₋₃ alkyl optionally substituted with 1-3 —F.
Embodiment 74. The compound of Embodiment 73, wherein the compound is other than Compound Nos. R302 or R356, as depicted in Table R1, or a pharmaceutically acceptable salt thereof.
Embodiment 75. The compound of Embodiment 73, wherein R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and/or
(5) a1a is 0, 1, or 2.
Embodiment 76. The compound of Embodiment 73, wherein R^{Yb} is C₁₋₃ alkoxy.
Embodiment 77. The compound of Embodiment 73 or 76, wherein R^{Yb} is OMe.
Embodiment 78. The compound of any one of Embodiments 47, 59, or 63-64, wherein the compound is a compound of Formula (I-i1) or (I-i2):

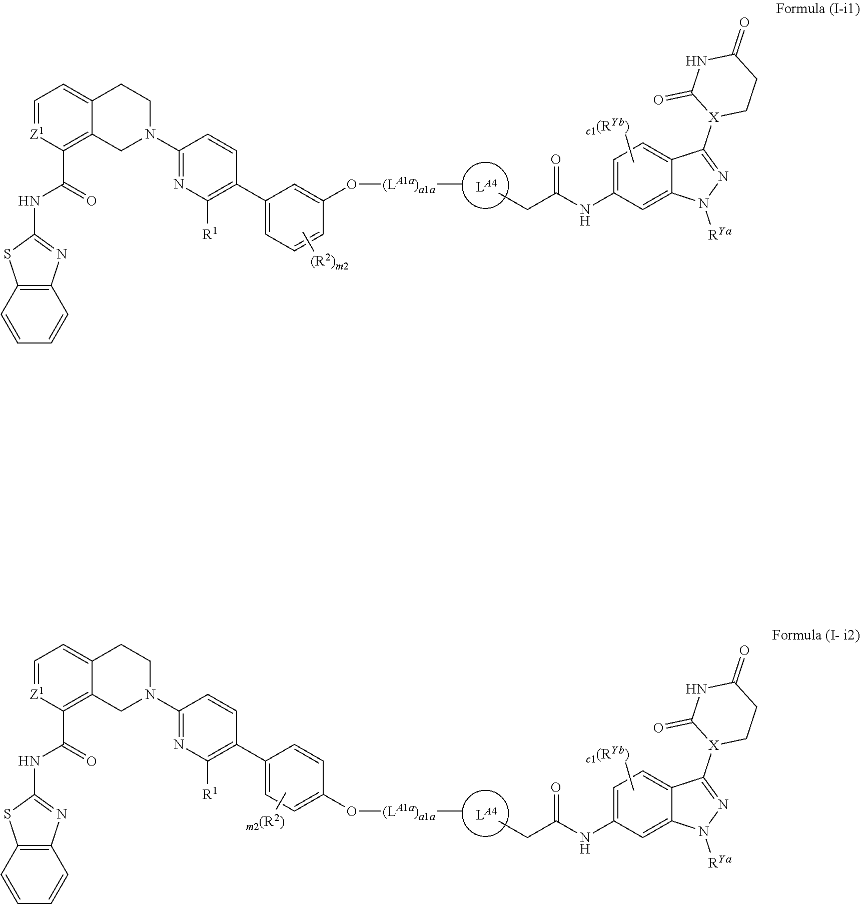

or a pharmaceutically acceptable salt thereof, wherein:
a1a is an integer from 0 to 4;
L^{A4} is

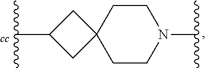

wherein cc represents point of attachment to -(L^{A1a})ₐ₁ₐ-.
m2 is 0 or 1; and
R² is CH₃ or CF₃.
Embodiment 79. The compound of Embodiment 78, wherein the compound is other than Compound Nos. R156 or R226, as depicted in Table R1, or a pharmaceutically acceptable salt thereof.
Embodiment 80. The compound of Embodiment 78, wherein the compound is a compound of Formula (I-i1), or a pharmaceutically acceptable salt thereof, and one of more of
(1)-(5) applies:
(1) m2 is 1, and R² is CF₃;
(2) a1a is 0;
(3) a1a is 2 or 3;
(4) c1 is 1 or 2; and/or
(5) at least one L^{A1a} is selected from the group consisting of CHR^{L} and C(R^{L})₂.
Embodiment 81. The compound of Embodiment 78, wherein the compound is a compound of Formula (I-i2), or a pharmaceutically acceptable salt thereof.
Embodiment 82. The compound of any one of Embodiments 47-81, wherein a1a is 0 or 1.
Embodiment 83. The compound of any one of Embodiments 47-81, wherein a1a is 2, 3, or 4 (e.g., 2 or 3).
Embodiment 84. The compound of any one of Embodiments 47-83, wherein each L^{A1a} is CH₂.
Embodiment 85. The compound of any one of Embodiments 47-83, wherein one L^{A1a} is CHR^{L}, wherein R^{L} is —F or C₁₋₃ alkyl optionally substituted with 1-3 F; and each remaining L^{A1a} is CH₂.
Embodiment 86. The compound of any one of Embodiments 47-58, 63-77, or 82-85, wherein L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 87. The compound of any one of Embodiments 47-58, 63-77, or 82-86, wherein L^{A4} is

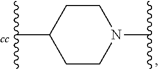

wherein cc represents the point of attachment to -(L^{A1a})ₐ₁ₐ-.
Embodiment 88. The compound of any one of Embodiments 47-58, 63-77, or 82-86, wherein L^{A4} is

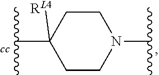

wherein R^{L4} is —F or —OH (e.g., —OH); and cc represents the point of attachment to -(L^{A1a})ₐ₁ₐ-.
Embodiment 89. The compound of any one of Embodiments 47-58, 63-77, or 82-85, wherein L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 90. The compound of any one of Embodiments 47-58, 63-77, 82-85, or 89, wherein L^{A4} is selected from the group consisting of:

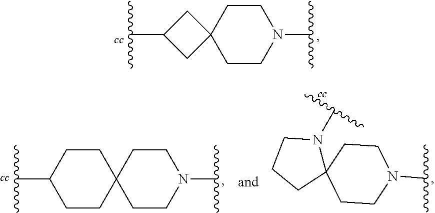 [SMILES: *C1CC2(CCN(*)CC2)C1 ; *C1CCC2(CC1)CCN(*)CC2 ; *N1CCC2(CCCN2*)CC1]

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F, and wherein cc represents the point of attachment to -(L^{A1a})ₐ₁ₐ-.
Embodiment 91. The compound of any one of Embodiments 47-58, 63-77, 82-85, or 89-90, wherein L^{A4} is

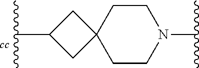

wherein cc represents the point of attachment to -(L^{A1a})ₐ₁ₐ-.
Embodiment 92. The compound of any one of Embodiments 47-50, 55-68, or 73-91, wherein m2 is 1; and R² is CF₃.
Embodiment 93. The compound of any one of Embodiments 47-50, 55-68, or 73-92, wherein the

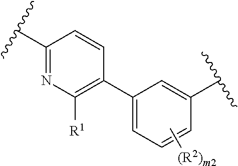

moiety is

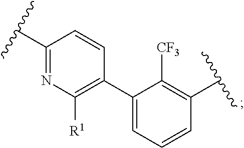

and
the

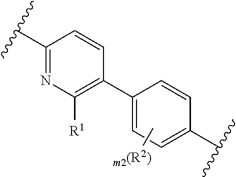

moiety is

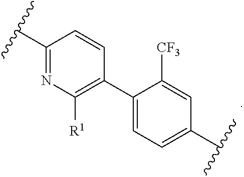

Embodiment 94. The compound of any one of Embodiments 47-50, 55-68, or 73-91, wherein m2 is 1; and R² is CH₃.
Embodiment 95. The compound of any one of Embodiments 47-50, 55-68, 73-91, or 94, wherein the

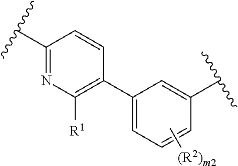

moiety is

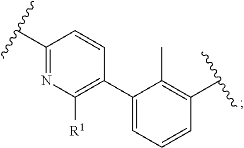

and
the

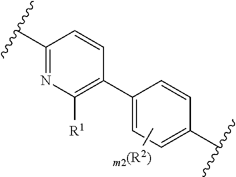

moiety is

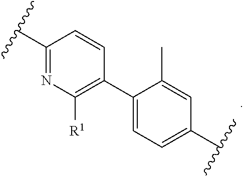

Embodiment 96. The compound of any one of Embodiments 47-49, 51-67, or 69-95, wherein Z¹ is CH.
Embodiment 97. The compound of any one of Embodiments 47-49, 51-67, or 69-95, wherein Z¹ is N.
Embodiment 98. The compound of any one of Embodiments 47-54, 59-64, or 82-97, wherein Ring C is

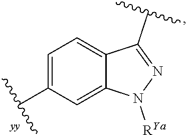

wherein R^{Ya} is C₁₋₃ alkyl optionally substituted with 1-3 R^{c}; and yy represents the point of attachment to -(L^{A3b})_{a3b}-.
Embodiment 99. The compound of any one of Embodiments 65-72 or 78-97, wherein the

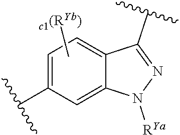

moiety is

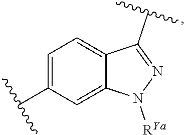

wherein R^{Ya} is C₁₋₃ alkyl optionally substituted with 1-3 R^{c}.
Embodiment 100. The compound of Embodiment 98 or 99, wherein R^{Ya} is methyl.
Embodiment 101. The compound of any one of Embodiments 47-54, 59-64, or 82-97, wherein Ring C is

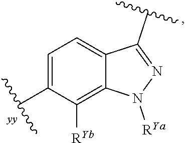

wherein R^{Ya} is C₁₋₃ alkyl optionally substituted with 1-3 R^{c};
R^{Yb} is selected from the group consisting of: —F, C₁₋₃ alkoxy, and C₁₋₃ alkyl optionally substituted with 1-3 F; and
yy represents the point of attachment to -(L^{A3b})_{a3b}-.
Embodiment 102. The compound of any one of Embodiments 65-72 or 78-97, wherein the

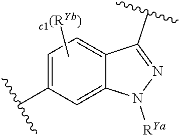

moiety is

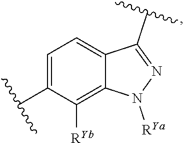

wherein R^{Ya} is C₁₋₃ alkyl optionally substituted with 1-3 R^{c}; and
R^{Yb} is selected from the group consisting of: —F, C₁₋₃ alkoxy, and C₁₋₃ alkyl optionally substituted with 1-3 F.
Embodiment 103. The compound of Embodiment 101 or 102, wherein R^{Ya} is methyl.
Embodiment 104. The compound of any one of Embodiments 101-103, wherein R^{Yb} is C₁₋₃ alkoxy.
Embodiment 105. The compound of any one of Embodiments 101-103, wherein R^{Yb} is selected from the group consisting of: —F, CH₃, and OMe.
Embodiment 106. The compound of any one of Embodiments 101-105, wherein R^{Yb} is OMe.
Embodiment 107. The compound of any one of Embodiments 1-106, wherein R¹ is C(O)OH.
Embodiment 108. The compound of any one of Embodiments 1-107, wherein X is CH.
Embodiment 109. The compound of any one of Embodiments 1-107, wherein X is N.
Embodiment 110. The compound of any one of Embodiments 1-109, wherein the compound is selected from the group consisting of Compound Nos. 101, 103, 104, 105, 106, 107, 108, 109, 112, 113, 114, 115, 116, 117, 118, 119, 120, 121, 122, 123, 124, 125, 126, 127, 128, 129, 130, 130a, 130b, 131, 132, 133, 133a, 133b, 134, 135, 136, 137, 138, 138a, 138b, 139, 140, 140a, 140b, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 173a, 174, 174a, 175, 175a, 176, 176a, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 208a, 208b, 208c, 208d, 209, 209a, 209b, 209c, 209d, 210, 210a, 210b, 210c, and 210d as depicted in Table C1, or a pharmaceutically acceptable salt thereof.
Embodiment 111. A pharmaceutical composition comprising a compound of any one of Embodiments 1-110, or a pharmaceutically acceptable salt thereof, and a pharmaceutically acceptable excipient.
Embodiment 112. A BCL-X_{L} protein non-covalently bound with a compound of any one of Embodiments 1-110, or a pharmaceutically acceptable salt thereof.
Embodiment 113. A ternary complex comprising a BCL-X_{L} protein, a compound of any one of Embodiments 1-110, or a pharmaceutically acceptable salt thereof, and a CRBN protein.
Embodiment 114. A method for treating a cancer in a subject in need thereof, the method comprising administering to the subject a therapeutically effective amount of a compound of any one of Embodiments 1-110, or a pharmaceutically acceptable salt thereof, or a pharmaceutical composition according to Embodiment 111.
Embodiment 115. The method of Embodiment 114, further comprising administering an additional therapy or therapeutic agent.
Embodiment 116. The method of Embodiment 115, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody, an ERK inhibitor, a FGFR1 inhibitor, a FGFR2 inhibitor, a FGFR3 inhibitor, a FGFR4 inhibitor, a HER2 inhibitor, a JAK2 inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, a TpoR agonist, chemotherapy, radiotherapy, or a combination thereof.
Embodiment 117. The method of any one of Embodiments 114-116, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.
Embodiment 118. The method of Embodiment 117, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.
Embodiment 119. A method for inducing degradation of a BCL-X_{L} protein in a mammalian cell, the method comprising contacting the mammalian cell with an effective amount of a compound of any one of Embodiments 1-110, or a pharmaceutically acceptable salt thereof.
Embodiment 120. The method of Embodiment 119, wherein the contacting occurs in vivo.
Embodiment 121. The method of Embodiment 119, wherein the contacting occurs in vitro.
Embodiment 122. The method of any one of Embodiments 119-121, wherein the mammalian cell is a mammalian cancer cell.

### Exemplary P03 Embodiments

Embodiment 1. A compound of Formula (I)

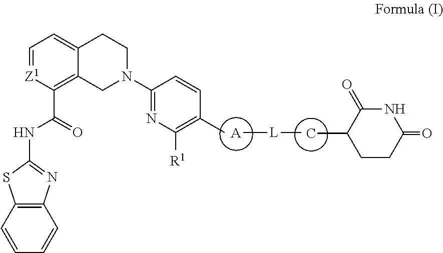

or a pharmaceutically acceptable salt thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{a};
Ring A is selected from the group consisting of: (A1) and (A2):

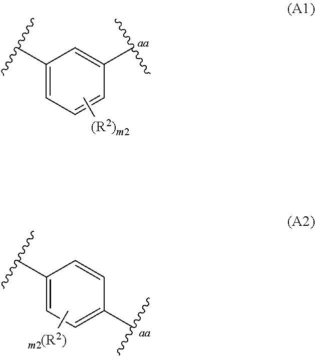

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to L;
L is -(L^{A})ₙ₁-, wherein:
n1 is an integer from 4 to 10,
one L^{A} is L^{A4};
0-3 L^{A} are independently selected L^{A3}, and
each remaining L^{A} is an independently selected L^{A1};
each L^{A1} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c};
each L^{A3} is independently selected from the group consisting of: —N(R^{d})—, —N(R^{b})—, —O—, —S(O)₀₋₂—, and C(═O);
L^{A4} is independently selected from the group consisting of:
(a) C₃₋₁₂ cycloalkylene or 4-15 membered heterocyclylene, each of which is optionally substituted with 1-3 R^{L4}; and
(b) phenylene or 5-6 membered heteroarylene, each of which is optionally substituted with 1-3 R^{L4};
wherein each R^{L4} is independently selected from the group consisting of R^{a} and R^{b};
Ring C is selected from the group consisting of:

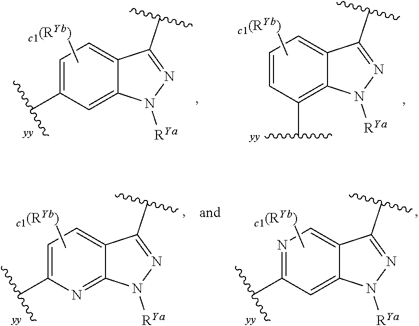

wherein:
R^{Ya} is selected from the group consisting of: H, R^{b}, and C₁₋₆ alkyl optionally substituted with 1-3 R^{c},
each R^{Yb} is independently selected from the group consisting of R^{a} and R^{b};
c1 is 0, 1, or 2; and
yy represents the point of attachment to L;
each R^{a} is independently selected from the group consisting of:
(a) halo;
(b) CN;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)OC₁₋₆ alkyl;
(i) C(═O)N(R^{f})₂;
(j) S(O)₀₋₂(C₁₋₆ alkyl);
(k) S(O)₀₋₂(C₁₋₆ haloalkyl); and
(l) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1 or 2;
each L^{b} is independently selected from the group consisting of: —O—, —N(H)—, —N(C₁₋₃ alkyl)-, —S(O)₀₋₂—, C(═O), and C₁₋₃ alkylene; and
each R1 is independently selected from the group consisting of: C₃₋₆ cycloalkyl and 4-8 membered heterocyclyl, each of which is optionally substituted with 1-3 R^{g};
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R^{f})₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.
Embodiment 2. The compound of Embodiment 1, wherein the compound of Formula (I) is other than a compound depicted in Table R1, or a pharmaceutically acceptable salt thereof.
Embodiment 3. The compound of Embodiment 1 or 2, wherein Z¹ is N.
Embodiment 4. The compound of Embodiment 1 or 2, wherein Z¹ is CH.
Embodiment 5. The compound of any one of Embodiments 1-4, wherein m2 is 1.
Embodiment 6. The compound of any one of Embodiments 1-5, wherein Ring A is (A1).
Embodiment 7. The compound of any one of Embodiments 1-5, wherein Ring A is (A2).
Embodiment 8. The compound of any one of Embodiments 1-6, wherein Ring A is

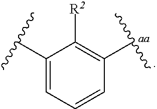

Embodiment 9. The compound of any one of Embodiments 1-5 or 7, wherein Ring A is

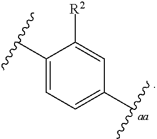

Embodiment 10. The compound of any one of Embodiments 1-9, wherein R² is C₁₋₃ alkyl optionally substituted with 1-3 R^{c} (e.g., C₁₋₃ alkyl optionally substituted with 1-3 F).
Embodiment 11. The compound of any one of Embodiments 1-10, wherein R² is CH₃.
Embodiment 12. The compound of any one of Embodiments 1-10, wherein R² is CF₃.
Embodiment 13. The compound of any one of Embodiments 1-9, wherein R² is —F.
Embodiment 14. The compound of any one of Embodiments 1-6, wherein Ring A is:

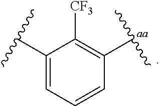 [SMILES: *c1cccc(*)c1C(F)(F)F]

Embodiment 15. The compound of any one of Embodiments 1-6, wherein Ring A is

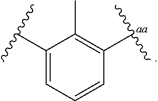 [SMILES: *c1cccc(*)c1C]

Embodiment 16. The compound of any one of Embodiments 1-6, wherein Ring A is

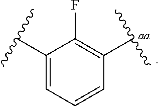 [SMILES: *c1cccc(*)c1F]

Embodiment 17. The compound of any one of Embodiments 1-5 or 7, wherein Ring A is:

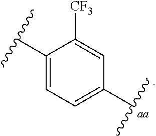 [SMILES: *c1ccc(*)c(C(F)(F)F)c1]

Embodiment 18. The compound of any one of Embodiments 1-5 or 7, wherein Ring A is:

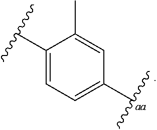 [SMILES: *c1ccc(*)c(C)c1]

Embodiment 19. The compound of any one of Embodiments 1-5 or 7, wherein Ring A is:

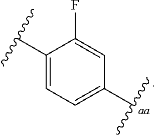 [SMILES: *c1ccc(*)c(F)c1]

Embodiment 20. The compound of any one of Embodiments 1-19, wherein n1 is an integer from 5 to 9 (e.g., 5, 6, 7, or 8).
Embodiment 21. The compound of any one of Embodiments 1-20, wherein n1 is an integer from 5 to 9; and L is:

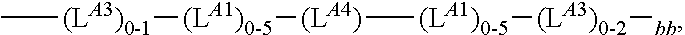

wherein bb represents the point of attachment to Ring C.
Embodiment 22. The compound of any one of Embodiments 1-21, wherein n1 is an integer from 5 to 9; and L is selected from the group consisting of:

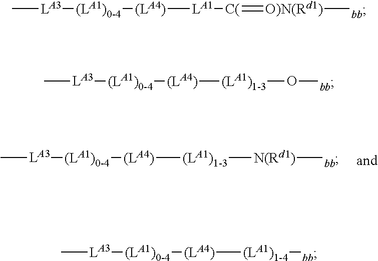

wherein bb represents the point of attachment to Ring C; and R^{d1} is H or C₁₋₃ alkyl.
Embodiment 23. The compound of any one of Embodiments 1-22, wherein n1 is an integer from 5 to 9; and L is:

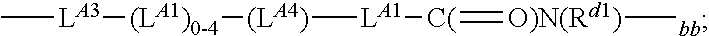

wherein bb represents the point of attachment to Ring C; and R^{d1} is H or C₁₋₃ alkyl.
Embodiment 24. The compound of any one of Embodiments 1-23, wherein n1 is an integer from 5 to 9; and L is:

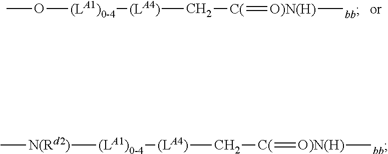

wherein bb represents the point of attachment to Ring C; and R^{d2} is H or C₁₋₃ alkyl.
Embodiment 25. The compound of any one of Embodiments 1-24, wherein L^{A4} is a 5-12 membered nitrogen-containing heterocyclylene optionally substituted with 1-3 R^{L4}
Embodiment 26. The compound of any one of Embodiments 1-25, wherein each R^{L4} is independently selected from the group consisting of: —F, CN, OH, C₁₋₃ alkoxy, and C₁₋₃ alkyl optionally substituted with 1-3 F.
Embodiment 27. The compound of any one of Embodiments 1-24, wherein L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 28. The compound of any one of Embodiments 1-24 or 27, wherein L^{A4} is

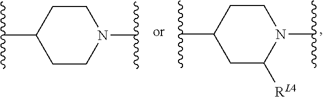

wherein R^{L4} is C₁₋₃ alkyl optionally substituted with 1-3 F.
Embodiment 29. The compound of any one of Embodiments 1-24 or 27, wherein L^{A4} is

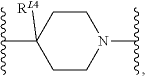

wherein R^{L4} is —F or —OH (e.g., —OH).
Embodiment 30. The compound of any one of Embodiments 1-24, wherein L^{A4} is a bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 31. The compound of any one of Embodiments 1-24 or 30, wherein L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 32. The compound of any one of Embodiments 1-24 or 30-31, wherein L^{A4} is selected from the group consisting of:

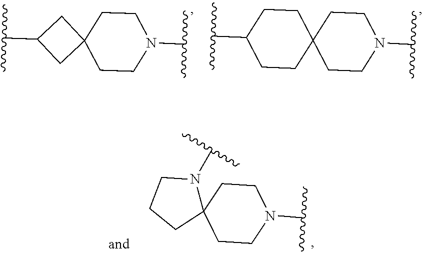

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 33. The compound of any one of Embodiments 1-24 or 30-32, wherein L^{A4} is

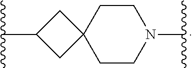 [SMILES: *C1CC2(CCN(*)CC2)C1]

Embodiment 34. The compound of any one of Embodiments 1-24 or 30, wherein L^{A4} is a bridged or fused bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 35. The compound of any one of Embodiments 1-24, 30, or 34, wherein L^{A4} is selected from the group consisting of:

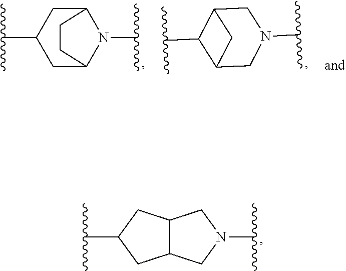

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 36. The compound of any one of Embodiments 1-35, wherein each L^{A1} is CH₂.
Embodiment 37. The compound of any one of Embodiments 1-35, wherein one L^{A1} is CHR^{L} or C(R^{L})₂, wherein each R^{L} is independently —F or C₁₋₃ alkyl optionally substituted with 1-3 F; and each remaining L^{A1} is CH₂.
Embodiment 38. The compound of any one of Embodiments 1-37, wherein Ring C is

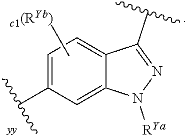

Embodiment 39. The compound of any one of Embodiments 1-38, wherein Ring C is

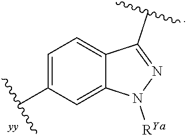

Embodiment 40. The compound of any one of Embodiments 1-38, wherein Ring C is

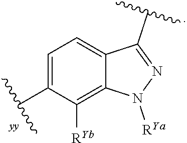

Embodiment 41. The compound of any one of Embodiments 1-38, wherein Ring C is

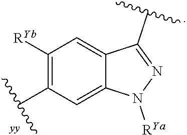

Embodiment 42. The compound of any one of Embodiments 1-41, wherein R^{Ya} is C₁₋₃ alkyl optionally substituted with 1-3 R^{c} (e.g., C₁₋₃ alkyl optionally substituted with 1-3 F).
Embodiment 43. The compound of any one of Embodiments 1-42, wherein R^{Ya} is methyl.
Embodiment 44. The compound of any one of Embodiments 1-38 or 40-43, wherein each R^{Yb} is independently selected from the group consisting of: —F, C₁₋₃ alkoxy, and C₁₋₃ alkyl optionally substituted with 1-3 F.
Embodiment 45. The compound of any one of Embodiments 1-38 or 40-44, wherein each R^{Yb} is independently selected from the group consisting of: —F, OMe, CH₃, CHF₂, CH₂F, and CF₃.
Embodiment 46. The compound of any one of Embodiments 1-38 or 40-44, wherein at least one R^{Yb} is C₁₋₃ alkoxy (e.g., OMe).
Embodiment 47. The compound of any one of Embodiments 1-37, wherein Ring C is

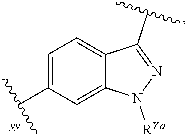

wherein R^{Ya} is C₁₋₃ alkyl optionally substituted with 1-3 R^{c} (e.g., C₁₋₃ alkyl optionally substituted with 1-3 F).
Embodiment 48. The compound of any one of Embodiments 1-37, wherein Ring C is

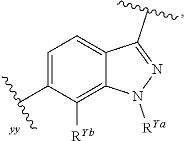

wherein R^{Ya} is C₁₋₃ alkyl optionally substituted with 1-3 R^{c} (e.g., C₁₋₃ alkyl optionally substituted with 1-3 F); and
R^{Yb} is independently selected from the group consisting of: —F, C₁₋₃ alkoxy, and C₁₋₃ alkyl optionally substituted with 1-3 F.
Embodiment 49. The compound of Embodiment 48, wherein R^{Yb} is C₁₋₃ alkoxy (e.g., methoxy).
Embodiment 50. The compound of Embodiment 1 or 21, wherein the compound is a compound of Formula (I-1) or (I-2):

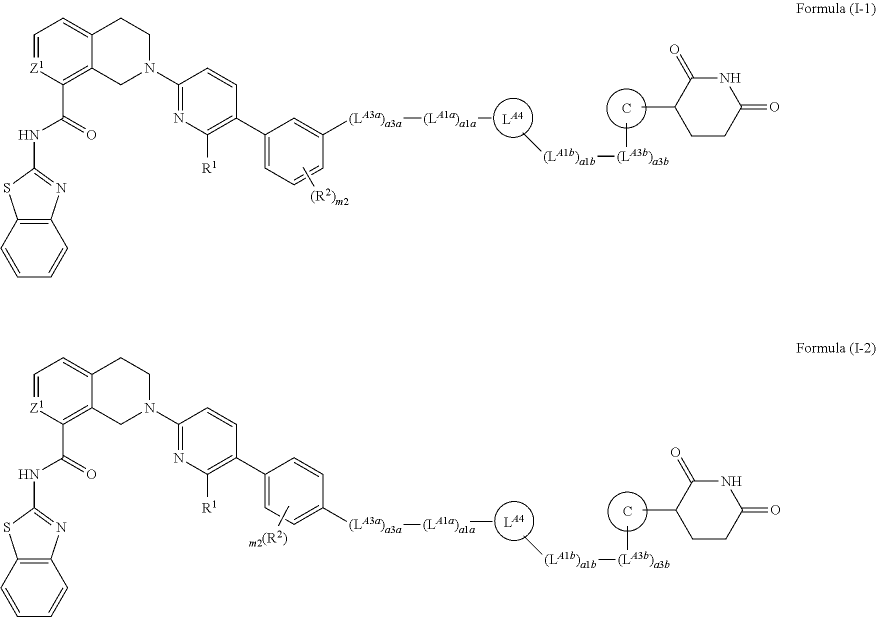

or a pharmaceutically acceptable salt thereof, wherein:
m2 is 0 or 1;
each L^{A3a} and L^{A3b} is an independently selected L^{A3};
a3a is 0 or 1;
a3b is 0, 1, or 2;
each L^{A1a} and L^{A1}b is an independently selected L^{A1};
a1a and a1b are independently integers from 0 to 4;
provided that: a3a+a1a+a1b+a3b is from 4 to 8; and
L^{A4} is a 5-12 membered nitrogen-containing heterocyclylene optionally substituted with 1-3 R^{L4};
Embodiment 51. The compound of Embodiment 50, wherein the compound is a compound of Formula (I-1), or a pharmaceutically acceptable salt thereof; and one or more of (1)-(4) applies:
(1) Z¹ is N;
(2) Ring C is

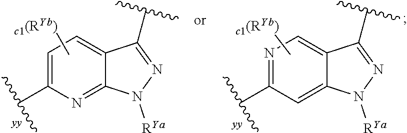

(3) c1 is 1 or 2, wherein at least one R^{Yb} is C₁₋₃ alkoxy (e.g., OMe); and
(4) a3a is 1, and L^{A3a} is —N(H)— or —N(C₁₋₃ alkyl)-.
Embodiment 52. The compound of Embodiment 50, wherein the compound is a compound of Formula (I-2), or a pharmaceutically acceptable salt thereof; and one or more of (1)-(5) applies:
(1) Z¹ is N;
(2) Ring C is

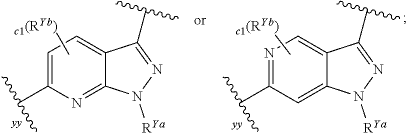

(3) c1 is 1 or 2, wherein at least one R^{Yb} is C₁₋₃ alkoxy; (4) m2 is 1; and R² is CF₃; and
(5) a3a is 1, and L^{A3a} is —N(H)— or —N(C₁₋₃ alkyl)-.
Embodiment 53. The compound of Embodiment 50, wherein the compound is a compound of Formula (I-a1) or (I-a2):

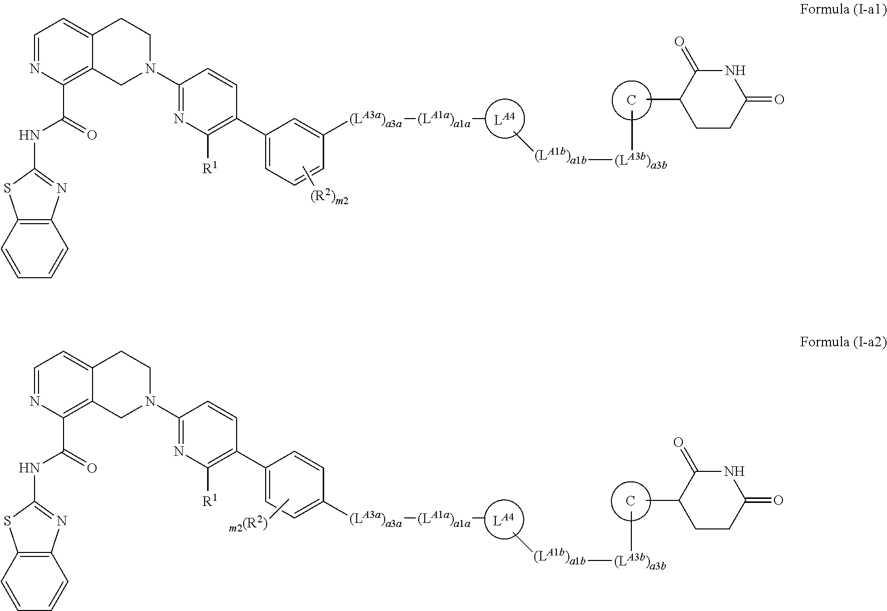

or a pharmaceutically acceptable salt thereof.
Embodiment 54. The compound of Embodiment 50, wherein the compound is a compound of Formula (I-b1), (I-b2), (I-b3), or (I-b4):

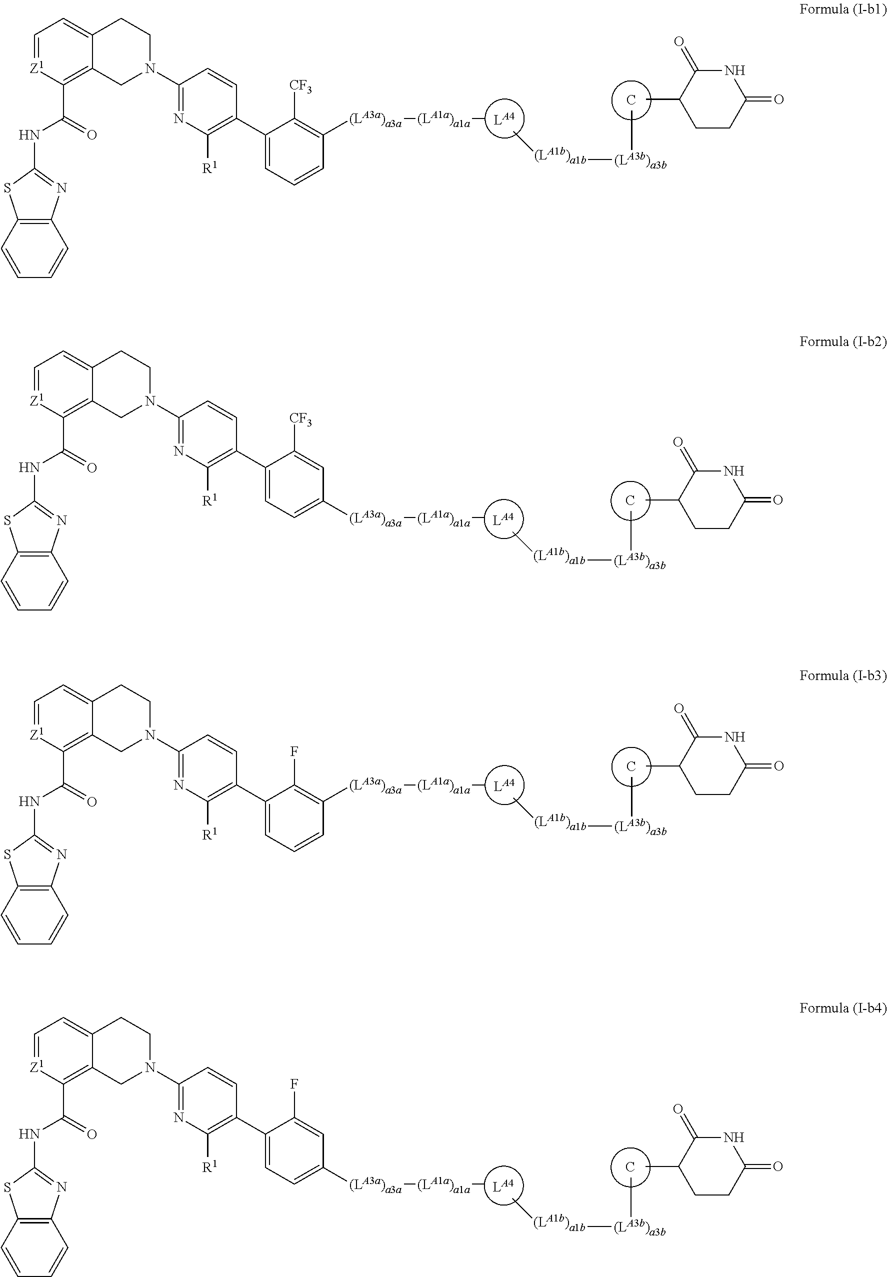

or a pharmaceutically acceptable salt thereof.
Embodiment 55. The compound of Embodiment 54, wherein the compound is other than Compound Nos. R126 or R313 as depicted in Table R1, or a pharmaceutically acceptable salt thereof.
Embodiment 56. The compound of Embodiment 54, wherein the compound is a compound of Formula (I-b2), (I-b3), or (I-b4), or a pharmaceutically acceptable salt thereof.
Embodiment 57. The compound of Embodiment 54, wherein the compound is a compound of Formula (I-b1), or a pharmaceutically acceptable salt thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and
(5) a1a is 0 or 1.
Embodiment 58. The compound of Embodiment 50, wherein the compound is a compound of Formula (I-c1) or (I-c2):

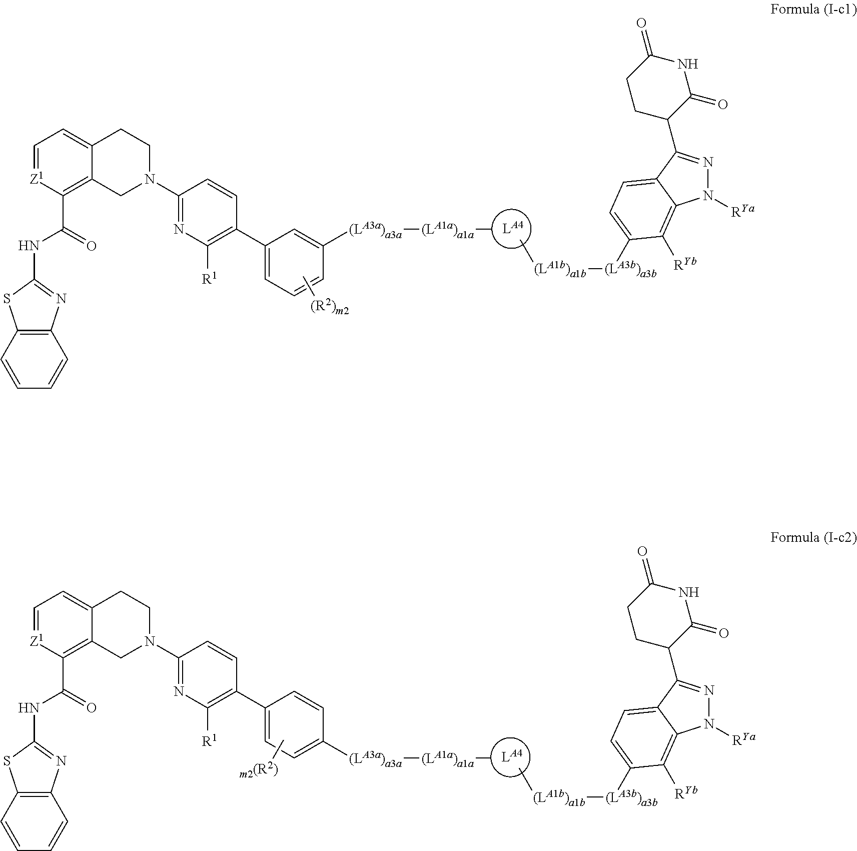

or a pharmaceutically acceptable salt thereof, wherein:
R^{Yb} is F, C₁₋₃ alkoxy, or C₁₋₃ alkyl optionally substituted with 1-3 —F.
Embodiment 59. The compound of Embodiment 58, wherein the compound is other than Compound Nos. R302 or R356 as depicted in Table R1, or a pharmaceutically acceptable salt thereof.
Embodiment 60. The compound of Embodiment 58, wherein R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and
(5) a1a is 0, 1, or 2.
Embodiment 61. The compound of Embodiment 58, wherein R^{Yb} is C₁₋₃ alkoxy (e.g., OMe).
Embodiment 62. The compound of Embodiment 50, wherein the compound is a compound of Formula (I-d1) or (I-d2):

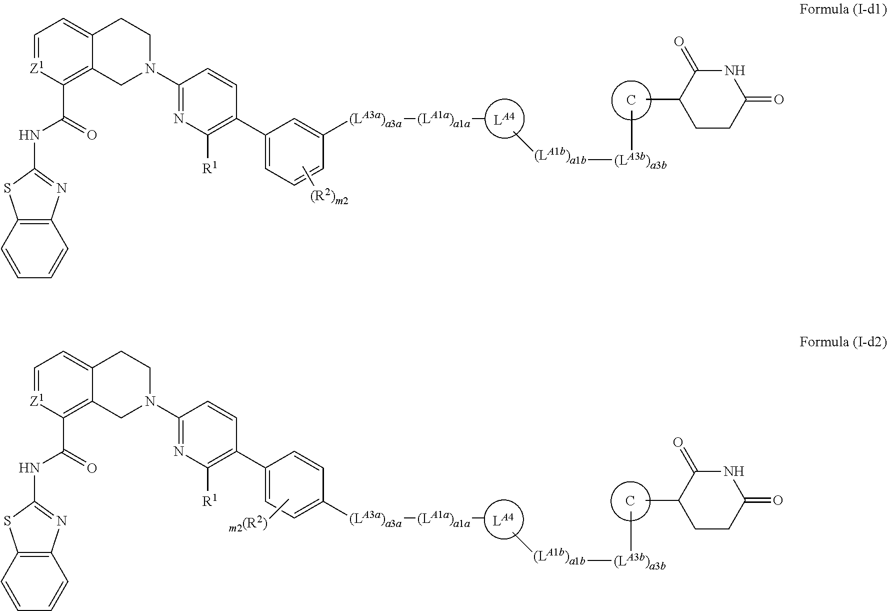

or a pharmaceutically acceptable salt thereof, wherein:
L^{A4} is

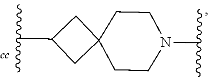

wherein cc represents point of attachment to -(L^{A1a})ₐ₁ₐ-.
Embodiment 63. The compound of Embodiment 62, wherein the compound is other than Compound Nos. R156 or R226 as depicted in Table R1, or a pharmaceutically acceptable salt thereof.
Embodiment 64. The compound of Embodiment 62, wherein the compound is a compound of Formula (I-d1), or a pharmaceutically acceptable salt thereof; and one of more of (1)-(5) applies:
(1) m2 is 1, and R² is CF₃;
(2) a1a is 0;
(3) a1a is 2 or 3;
(4) c1 is 1 or 2; and
(5) at least one L^{A1a} is selected from the group consisting of CHR^{L} and C(R^{L})₂.
Embodiment 65. The compound of Embodiment 62, wherein the compound is a compound of Formula (I-d2), or a pharmaceutically acceptable salt thereof.
Embodiment 66. The compound of any one of Embodiments 50-65, wherein alb is 1; and L^{A1}b is —CH₂—.
Embodiment 67. The compound of any one of Embodiments 50-66, wherein a3a is 1; L^{A3a} is —O—; a3b is 2; and -(L^{A3}b)_{a3b}- is —C(═O)NH— that is attached to Ring C at the nitrogen atom.
Embodiment 68. The compound of any one of Embodiments 50-66, wherein a3a is 1; L^{A3a} is —N(R^{d2})—, wherein R^{d2} is —H or C₁₋₃ alkyl; a3b is 2; and -(L^{A3}b)_{a3b}- is —C(═O)NH— that is attached to Ring C at the nitrogen atom.
Embodiment 69. The compound of any one of Embodiments 50 or 66-67, wherein the compound is a compound of Formula (I-e1) or (I-e2):

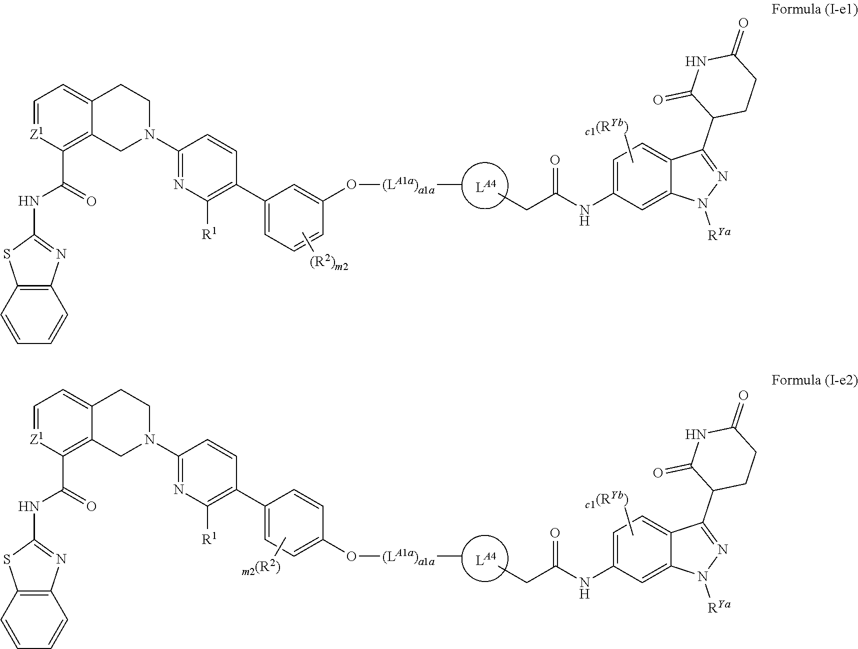

or a pharmaceutically acceptable salt thereof, wherein:
a1a is an integer from 0 to 4;
m2 is 0 or 1; and
R² is CH₃ or CF₃.
Embodiment 70. The compound of Embodiment 69, wherein the compound is a compound of Formula (I-e1), or a pharmaceutically acceptable salt thereof; and one or more of (1)-(3) applies:
(1) Z¹ is N;
(2) Ring C is

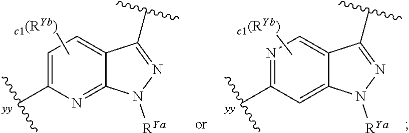

and
(3) c1 is 1 or 2, wherein at least one R^{Yb} is C₁₋₃ alkoxy.
Embodiment 71. The compound of Embodiment 69, wherein the compound is a compound of Formula (I-e2), or a pharmaceutically acceptable salt thereof; and one or more of (1)-(4) applies:
(1) Z¹ is N;
(2) Ring C is

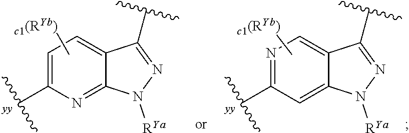

(3) c1 is 1 or 2, wherein at least one R^{Yb} is C₁₋₃ alkoxy; and
(4) m2 is 1; and R² is CF₃.
Embodiment 72. The compound of any one of Embodiments 50, 66, or 68, wherein the compound is a compound of Formula (I-e3) or (I-e4):

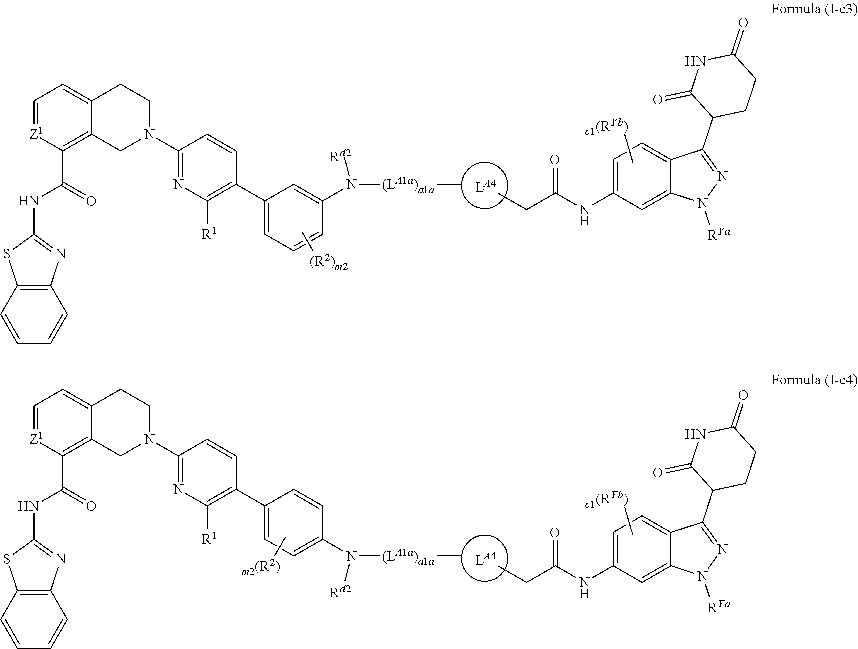

or a pharmaceutically acceptable salt thereof, wherein:
a1a is an integer from 0 to 4;
m2 is 0 or 1;
R² is CH₃, CF₃, or —F; and
R¹² is H or C₁₋₃ alkyl (e.g., H or methyl).
Embodiment 73. The compound of any one of Embodiments 50, 53, or 66-67, wherein the compound is a compound of Formula (I-f1) or (I-f2):

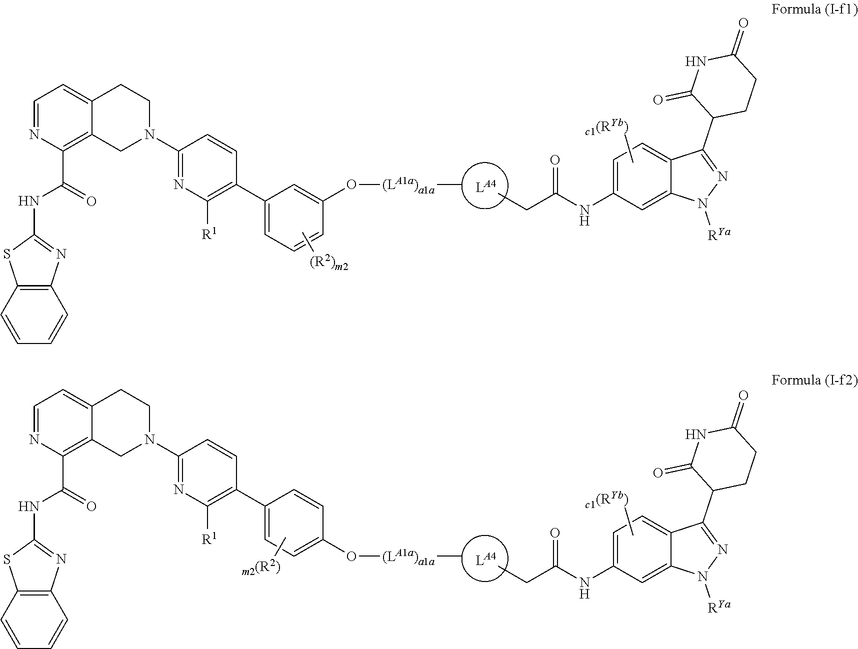

or a pharmaceutically acceptable salt thereof, wherein:
a1a is an integer from 0 to 4;
m2 is 0 or 1; and
R² is CH₃ or CF₃.
Embodiment 74. The compound of any one of Embodiments 50, 54, or 66-67, wherein the compound is a compound of Formula (I-g1), (I-g2), (I-g3), or (I-g4):

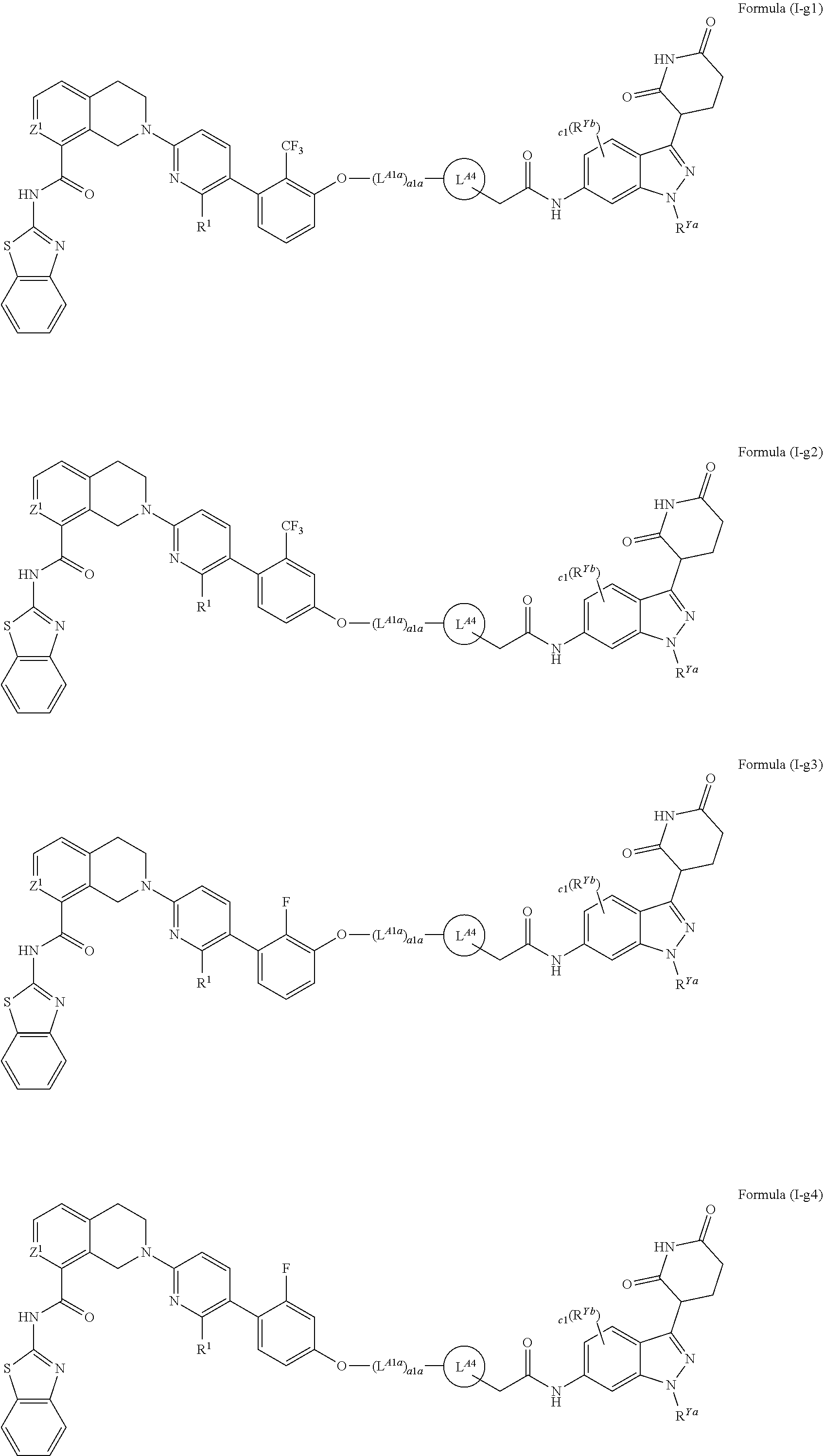

or a pharmaceutically acceptable salt thereof, wherein:
a1a is an integer from 0 to 4.
Embodiment 75. The compound of Embodiment 74, wherein the compound is a compound of Formula (I-g1); and the compound is other than Compound Nos. R126 or R313 as depicted in Table R1, or a pharmaceutically acceptable salt thereof.
Embodiment 76. The compound of Embodiment 74, wherein the compound is a compound of Formula (I-g2), (I-g3), or (I-g4), or a pharmaceutically acceptable salt thereof.
Embodiment 77. The compound of Embodiment 74, wherein the compound is a compound of Formula (I-g1), or a pharmaceutically acceptable salt thereof; and one or more of (1)-(5) applies:
(1) c1 is 1 or 2;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and
(5) a1a is 0 or 1.
Embodiment 78. The compound of any one of Embodiments 50, 58, or 66-67, wherein the compound is a compound of Formula (I-h1) or (I-h2):

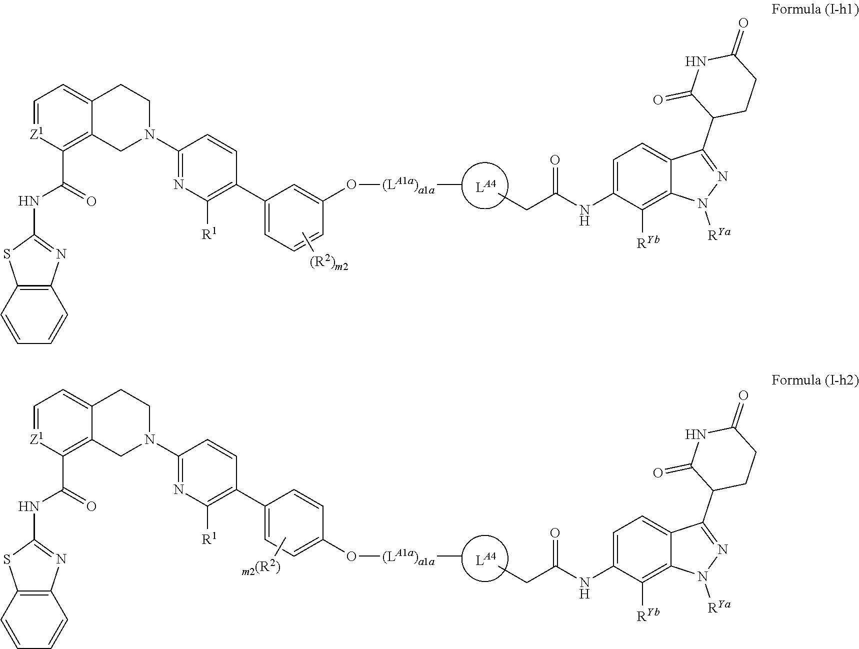

or a pharmaceutically acceptable salt thereof, wherein:
a1a is an integer from 0 to 4;
m2 is 0 or 1;
R² is CH₃ or CF₃; and
R^{Yb} is F, C₁₋₃ alkoxy, or C₁₋₃ alkyl optionally substituted with 1-3 —F.
Embodiment 79. The compound of Embodiment 78, wherein the compound is other than Compound Nos. R302 or R356 as depicted in Table R1, or a pharmaceutically acceptable salt thereof.
Embodiment 80. The compound of Embodiment 78, wherein R^{Yb} is F; and one or more of (1)-(5) applies:
(1) m2 is 1, and R² is CF₃;
(2) L^{A4} is a bicyclic 7-12 membered nitrogen-containing heterocyclylene optionally substituted with 1-3 R^{L4};
(3) L^{A4} is a 5-12 membered nitrogen-containing heterocyclylene substituted with 1-3 R^{L4};
(4) at least one L^{A1a} is selected from the group consisting of CHR^{L} and C(R^{L})₂; and
(5) a1a is 0, 1, or 2.
Embodiment 81. The compound of Embodiment 78, wherein R^{Yb} is C₁₋₃ alkoxy.
Embodiment 82. The compound of Embodiment 78 or 80, wherein R^{Yb} is OMe.
Embodiment 83. The compound of any one of Embodiments 50, 62, or 66-67, wherein the compound is a compound of Formula (I-i1), (I-i2), or (I-i3):

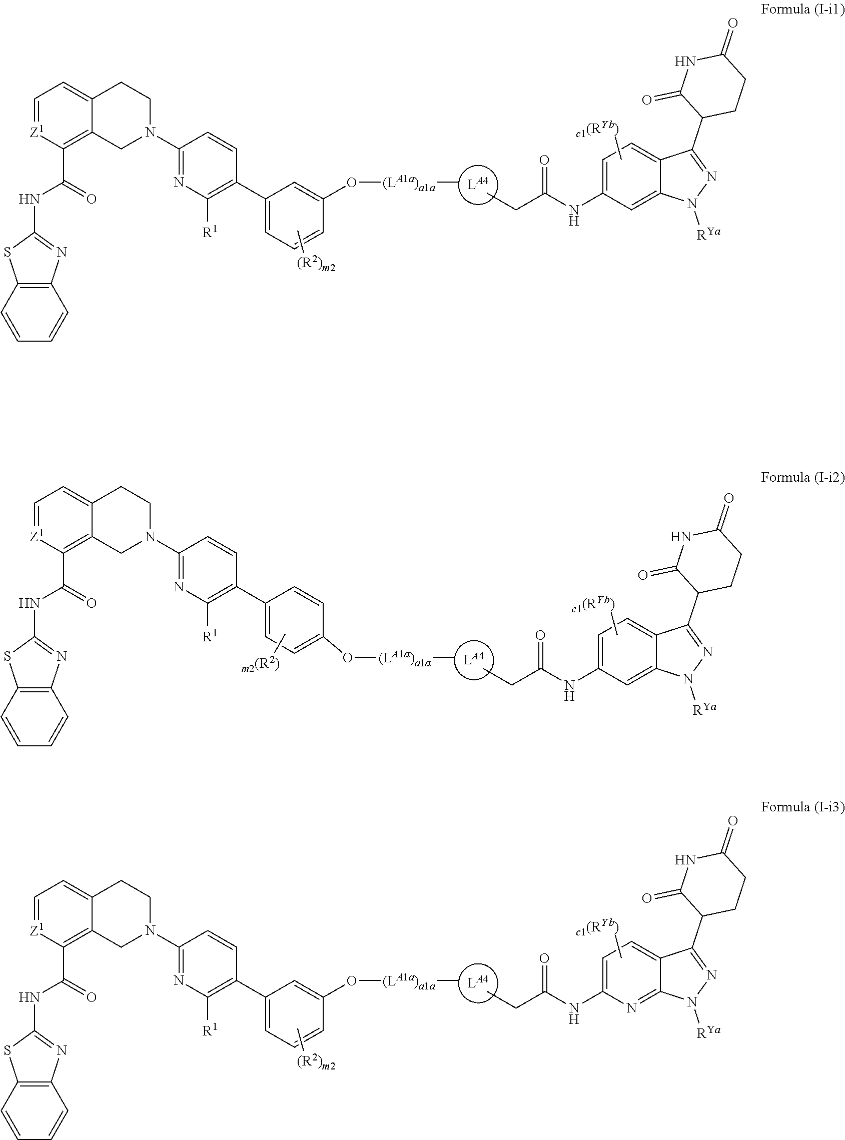

or a pharmaceutically acceptable salt thereof, wherein:
a1a is an integer from 0 to 4;
L^{A4} is

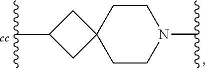

wherein cc represents point of attachment to -(L^{A1a})ₐ₁ₐ-.
m2 is O or 1; and
R² is CH₃ or CF₃.
Embodiment 84. The compound of Embodiment 83, wherein the compound is other than Compound Nos. R156 or R226 as depicted in Table R1, or a pharmaceutically acceptable salt thereof.
Embodiment 85. The compound of Embodiment 83, wherein the compound is a compound of Formula (I-i1), or a pharmaceutically acceptable salt thereof, and one of more of (1)-(5) applies:
(1) m2 is 1, and R² is CF₃;
(2) a1a is 0;
(3) a1a is 2 or 3;
(4) c1 is 1 or 2; and
(5) at least one L^{A1a} is selected from the group consisting of CHR^{L} and C(R^{L})₂.
Embodiment 86. The compound of Embodiment 83, wherein the compound is a compound of Formula (I-i2), or a pharmaceutically acceptable salt thereof.
Embodiment 87. The compound of any one of Embodiments 50-86, wherein a1a is 0 or 1.
Embodiment 88. The compound of any one of Embodiments 50-86, wherein a1a is 2, 3, or 4 (e.g., 2 or 3).
Embodiment 89. The compound of any one of Embodiments 50-88, wherein each L^{A1a} is CH₂.
Embodiment 90. The compound of any one of Embodiments 50-88, wherein one L^{A1a} is CHR^{L}, wherein R^{L} is —F or C₁₋₃ alkyl optionally substituted with 1-3 F; and each remaining L^{A1a} is CH₂.
Embodiment 91. The compound of any one of Embodiments 50-61, 66-82, or 87-90, wherein L^{A4} is a monocyclic 5-6 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 92. The compound of any one of Embodiments 50-61, 66-82, or 87-91, wherein L^{A4} is

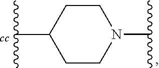

wherein cc represents the point of attachment to -(L^{A1a})ₐ₁ₐ-.
Embodiment 93. The compound of any one of Embodiments 50-61, 66-82, or 87-91, wherein L^{A4} is

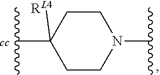

wherein R^{L4} is —F or —OH (e.g., —OH); and cc represents the point of attachment to -(L^{A1a})ₐ₁ₐ-.
Embodiment 94. The compound of any one of Embodiments 50-61, 66-82, or 87-91, wherein L^{A4} is

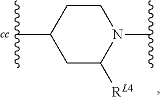

R^{L4}, wherein R^{L4} is C₁₋₃ alkyl optionally substituted with 1-3 F; and cc represents the point of attachment to -(L^{A1a})ₐ₁ₐ-.
Embodiment 95. The compound of any one of Embodiments 50-61, 66-82, or 87-90, wherein L^{A4} is a spirocyclic bicyclic 7-12 membered nitrogen-containing heterocyclylene optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F.
Embodiment 96. The compound of any one of Embodiments 50-61, 66-82, 87-90, or 95, wherein L^{A4} is selected from the group consisting of:

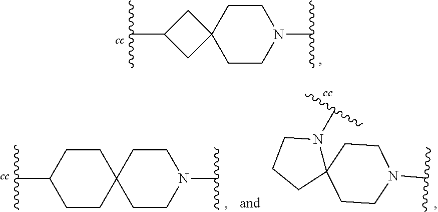 [SMILES: *C1CC2(CCN(*)CC2)C1 ; *C1CCC2(CC1)CCN(*)CC2 ; *N1CCC2(CCCN2*)CC1]

each of which is optionally substituted with 1-3 R^{L4}, wherein each R^{L4} is independently selected from the group consisting of: —F, CN, OH, C₁₋₃ alkoxy, and C₁₋₃ alkyl optionally substituted with 1-3 F, and wherein cc represents the point of attachment to -(L^{A1a})ₐ₁ₐ-.
Embodiment 97. The compound of any one of Embodiments 50-61, 66-82, 87-90, or 95-96, wherein L^{A4} is

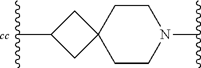

wherein cc represents the point of attachment to -(L^{A1a})ₐ₁ₐ-.
Embodiment 98. The compound of any one of Embodiments 50-53, 58-73, or 78-97, wherein m2 is 1; and R² is CF₃.
Embodiment 99. The compound of any one of Embodiments 50-53, 58-73, or 78-98, wherein the

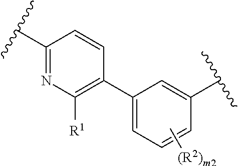

moiety is

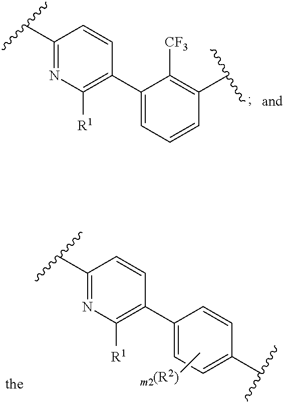

moiety is

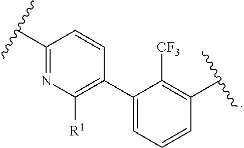

Embodiment 100. The compound of any one of Embodiments 50-53, 58-73, or 78-97, wherein m2 is 1; and R² is CH₃.
Embodiment 101. The compound of any one of Embodiments 50-53, 58-73, 78-97, or 100, wherein the

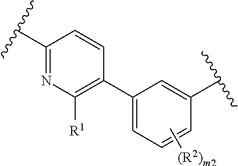

moiety is

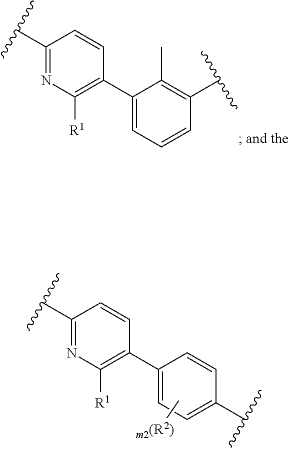

moiety is

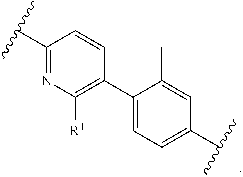

Embodiment 102. The compound of any one of Embodiments 50-52, 54-72, or 74-101, wherein Z¹ is CH.
Embodiment 103. The compound of any one of Embodiments 50-52, 54-72, or 74-101, wherein Z¹ is N.
Embodiment 104. The compound of any one of Embodiments 50-103, wherein each R^{Yb} is independently selected from the group consisting of: —F, C₁₋₃ alkoxy, and C₁₋₃ alkyl optionally substituted with 1-3 F; or R^{Yb} is independently selected from the group consisting of: —F, OMe, CH₃, CHF₂, CH₂F, and CF₃.
Embodiment 105. The compound of any one of Embodiments 50-57, 62-68, or 87-103, wherein Ring C is

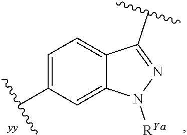

wherein R^{Ya} is C₁₋₃ alkyl optionally substituted with 1-3 R^{c}; and yy represents the point of attachment to -(L^{A3}b)_{a3b}- (e.g., R^{Ya} is methyl).
Embodiment 106. The compound of any one of Embodiments 69-77 or 87-103, wherein the

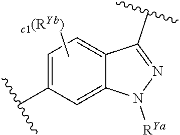

moiety is

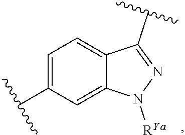

wherein R^{Ya} is C₁₋₃ alkyl optionally substituted with 1-3 R^{c} (e.g., R^{Ya} is methyl).
Embodiment 107. The compound of any one of Embodiments 50-57, 62-68, or 87-103, wherein Ring C is

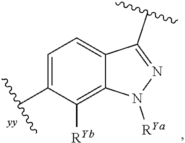

wherein R^{Ya} is C₁₋₃ alkyl optionally substituted with 1-3 R^{c};
R^{Yb} is selected from the group consisting of: —F, C₁₋₃ alkoxy, and C₁₋₃ alkyl optionally substituted with 1-3 F; and
yy represents the point of attachment to -(L^{A3b})_{a3b}-.
Embodiment 108. The compound of any one of Embodiments 69-77 or 83-103, wherein the

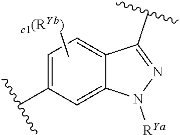

moiety is

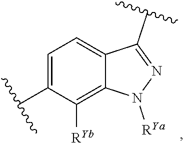

wherein R^{Ya} is C₁₋₃ alkyl optionally substituted with 1-3 R^{c}; and
R^{Yb} is selected from the group consisting of —F, C₁₋₃ alkoxy, and C₁₋₃ alkyl optionally substituted with 1-3 F.
Embodiment 109. The compound of Embodiment 107 or 108, wherein R^{Ya} is methyl.
Embodiment 110. The compound of any one of Embodiments 107-109, wherein R^{Yb} is C₁₋₃ alkoxy.
Embodiment 111. The compound of any one of Embodiments 107-109, wherein R^{Yb} is selected from the group consisting of: —F, CH₃, and OMe.
Embodiment 112. The compound of any one of Embodiments 107-111, wherein R^{Yb} is OMe.
Embodiment 113. The compound of any one of Embodiments 1-112, wherein R¹ is C(O)OH.
Embodiment 114. The compound of any one of Embodiments 1-113, wherein the compound is selected from the group consisting of the compounds depicted in Table C1, or a pharmaceutically acceptable salt thereof.
Embodiment 115. A compound of Formula (II)

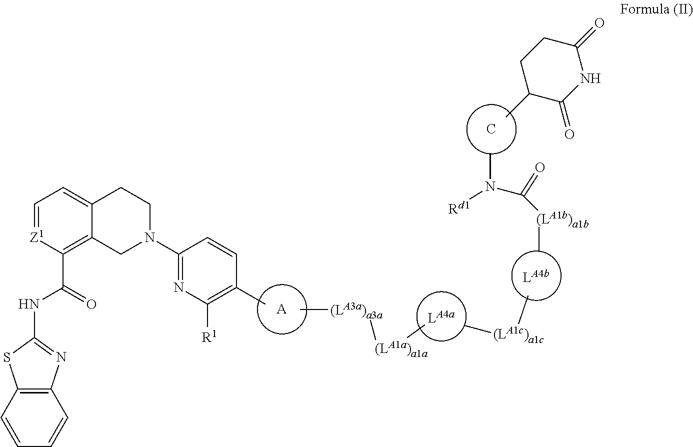

or a pharmaceutically acceptable salt thereof, wherein:
Z¹ is selected from the group consisting of: N and CH;
R¹ is selected from the group consisting of:
(a) C(O)OH;
(b) C(O)OC₁₋₆ alkyl, wherein the C₁₋₆ alkyl is optionally substituted with 1-3 R^{c}; and
(c) C(O)—(C₀₋₃ alkylene)-phenyl, wherein the phenyl is optionally substituted with 1-3 R^{g};
Ring A is selected from the group consisting of: (A1) and (A2):

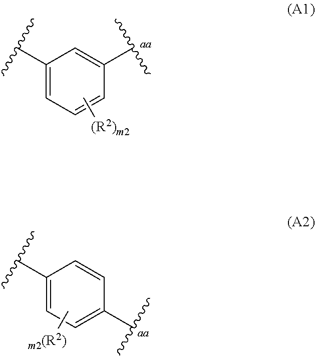

wherein:
m2 is 0, 1, or 2,
each R² is independently selected from the group consisting of: halo, CN, C₁₋₃ alkoxy, C₁₋₃ haloalkoxy, and C₁₋₃ alkyl optionally substituted with 1-3 R^{c}, and
aa represents the point of attachment to -(L^{A3a})ₐ₃ₐ-;
L^{A3a} is selected from the group consisting of: —N(R^{d})—, —O—, —S(O)₀₋₂—, and C(═O); a3a is 0 or 1;
each L^{A1a} L^{A1b}, and L^{A1c} is independently selected from the group consisting of: —CH₂—, —CHR^{L}—, and —C(R^{L})₂—, wherein:
each R^{L} is independently selected from the group consisting of: halo, —CN, —OH, —C₁₋₆ alkoxy, —C₁₋₆ haloalkoxy, —(C₀₋₃ alkylene)-(C₃₋₅ cycloalkyl), —(C₀₋₃ alkylene)-(4-6 membered heterocyclyl), and C₁₋₆ alkyl optionally substituted with 1-6 R^{c}; a1a, a1b, and a1c are each independently 0, 1, or 2;
L^{A4a} and L^{A}4b are independently selected from the group consisting of: C₃₋₈ cycloalkylene and 4-12 membered heterocyclylene, each of which is optionally substituted with 1-3 R^{L4};
wherein each R^{L4} is independently selected from the group consisting of: —F, CN, OH, C₁₋₃ alkoxy, and C₁₋₃ alkyl optionally substituted with 1-3 R^{c};
R^{d1} is —H or C₁₋₃ alkyl;
Ring C is selected from the group consisting of:

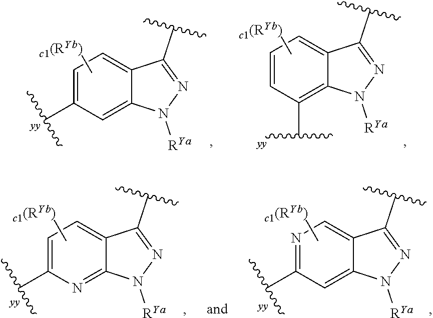

wherein:
R^{Ya} is selected from the group consisting of: H and C₁₋₆ alkyl optionally substituted with 1-3 R^{c}
each R^{Yb} is independently selected from the group consisting of: F, C₁₋₃ alkoxy, or C₁₋₃ alkyl optionally substituted with 1-3 R^{c};
c1 is 0, 1, or 2; and
yy represents the point of attachment to —N(R^{d1})—;
each R^{c} is independently selected from the group consisting of: halo, CN, —OH, —C₁₋₆ alkoxy, —C₁₋₆ haloalkoxy, —NR^{d}R^{e}, C(═O)C₁₋₆ alkyl, C(═O)OC₁₋₆ alkyl, C(═O)N(R₂, S(O)₀₋₂(C₁₋₆ alkyl), and S(O)₀₋₂(C₁₋₆ haloalkyl);
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h},
each R9 is independently selected from the group consisting of: R^{h}, C₁₋₃ alkyl, and C₁₋₃ haloalkyl; and
each R^{h} is independently selected from the group consisting of: halo, CN, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.
Embodiment 116. The compound of Embodiment 115, wherein Z¹ is CH.
Embodiment 117. The compound of Embodiment 115 or 116, wherein Ring A is

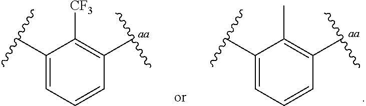

Embodiment 118. The compound of Embodiment 115 or 117, wherein Ring A is

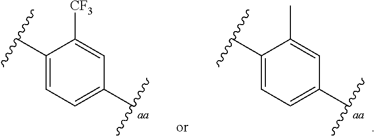

Embodiment 119. The compound of any one of Embodiments 115-118, wherein a3a is 1.
Embodiment 120. The compound of any one of Embodiments 115-119, wherein L^{A3}a is —O—.
Embodiment 121. The compound of any one of Embodiments 115-120, wherein a1a is 1.
Embodiment 122. The compound of any one of Embodiments 115-121, wherein L^{A1a} is —CH₂—.
Embodiment 123. The compound of any one of Embodiments 115-122, wherein a1c is 0 or 1.
Embodiment 124. The compound of any one of Embodiments 115-123, wherein L^{A}1c is —CH₂—.
Embodiment 125. The compound of any one of Embodiments 115-124, wherein a1b is 1.
Embodiment 126. The compound of any one of Embodiments 115-125, wherein L^{A}1b is —CH₂—.
Embodiment 127. The compound of any one of Embodiments 115-126, wherein R^{d1} is —H.
Embodiment 128. The compound of any one of Embodiments 115-127, wherein L^{A4b} is a 5-12 membered nitrogen-containing heterocyclylene optionally substituted with 1-3 R^{L4}
Embodiment 129. The compound of any one of Embodiments 115-129, wherein L^{A4b} is a monocyclic 5-6 membered nitrogen-containing heterocyclylene optionally substituted with 1-3 R^{L4}
Embodiment 130. The compound of any one of Embodiments 115-131, wherein L^{A4a} is a C₃₋₆ cycloalkylene optionally substituted with 1-2 R^{L4}
Embodiment 131. The compound of any one of Embodiments 115-130, wherein each R^{L4} is independently selected from the group consisting of: —F, CN, OH, C₁₋₃ alkoxy, and C₁₋₃ alkyl optionally substituted with 1-3 F.
Embodiment 132. The compound of any one of Embodiments 115-127, wherein L^{A4b} is

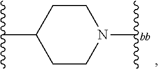

wherein bb represents point of attachment to -(L^{A1b})_{a1b}-.
Embodiment 133. The compound of any one of Embodiments 115-127 or 132, wherein L^{A4a} is

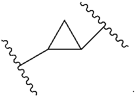 [SMILES: *C1CC1*]

Embodiment 134. The compound of any one of Embodiments 115-133, wherein Ring C is

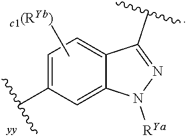

Embodiment 135. The compound of any one of Embodiments 115-134, wherein c1 is 0.
Embodiment 136. The compound of any one of Embodiments 115-135, wherein R^{Ya} is C₁₋₆ alkyl optionally substituted with 1-3 —F.
Embodiment 137. The compound of any one of Embodiments 115-136, wherein R^{Ya} is methyl.
Embodiment 138. The compound of any one of Embodiments 115-137, wherein R¹ is C(O)OH.
Embodiment 139. The compound of Embodiment 115, wherein the compound is a compound of Formula (II-a1) or (II-a2):

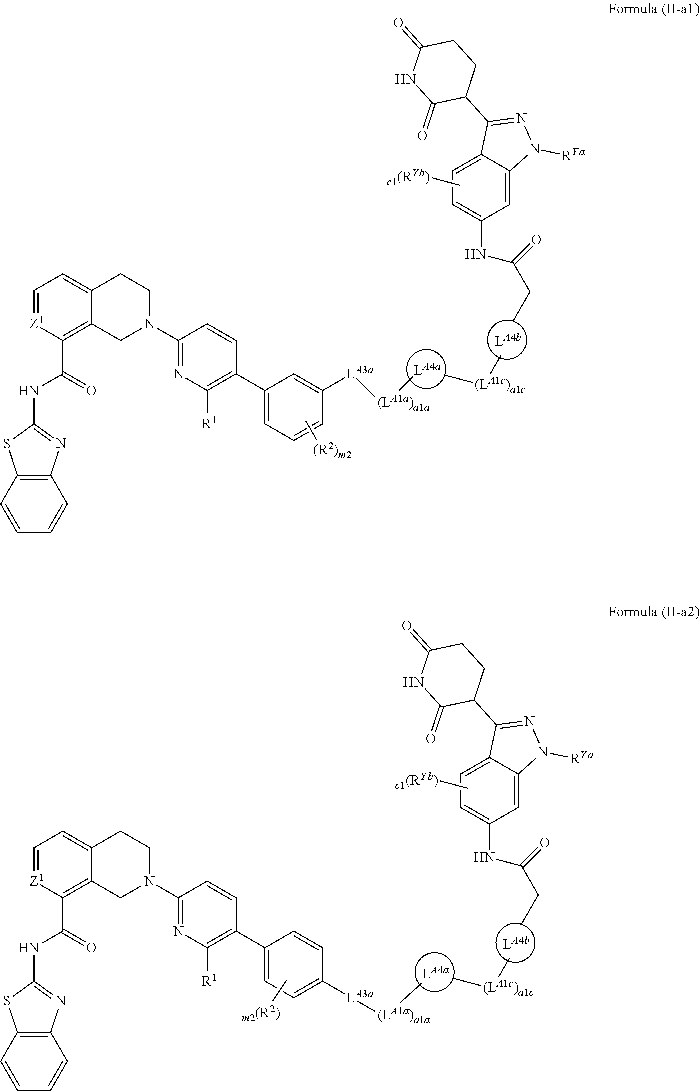

or a pharmaceutically acceptable salt thereof, wherein:
Z¹ is CH or N;
L^{A3a} is selected from the group consisting of: —N(R^{d})— and —O—;
each L^{A1a} and L^{A1c} is independently selected from the group consisting of: —CH₂—, —CHR^{L}—, and —C(R^{L})₂—, wherein:
each R^{L} is independently selected from the group consisting of: halo and C₁₋₃ alkyl optionally substituted with 1-3 —F;
a1a and a1c are each independently 0, 1, or 2;
L^{A4a} is a C₃₋₆ cycloalkylene optionally substituted with 1-2 R^{L4};
L^{A4b} is a monocyclic 5-6 membered nitrogen-containing heterocyclylene optionally substituted with 1-3 R^{L4}; and
each R^{L4} is independently selected from the group consisting of: —F, CN, OH, C₁₋₃ alkoxy, and C₁₋₃ alkyl optionally substituted with 1-3 F.
Embodiment 140. The compound of Embodiment 139, wherein a1a is 1; and L^{A1a} is —CH₂—.
Embodiment 141. The compound of Embodiment 139 or 140, wherein alc is 0 or 1.
Embodiment 142. The compound of any one of Embodiments 139-141, wherein L^{A1}c is —CH₂—.
Embodiment 143. The compound of any one of Embodiments 139-142, wherein L^{A4a} is

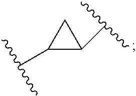 [SMILES: *C1CC1*]

and L^{A4b} is

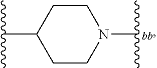

wherein bb represents point of attachment to

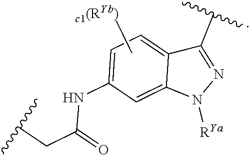

Embodiment 144. The compound of any one of Embodiments 139-143, wherein c1 is 0; and R^{Ya} is C₁₋₃ alkyl (e.g., methyl).
Embodiment 145. The compound of any one of Embodiments 139-144, wherein m2 is 1; and R² is CH₃ or CF₃.
Embodiment 146. The compound of any one of Embodiments 139-145, wherein Z¹ is CH.
Embodiment 147. The compound of any one of Embodiments 139-146, wherein R¹ is C(O)OH.
Embodiment 148. The compound of any one of Embodiments 115-147, wherein the compound is selected from the group consisting of the compounds depicted in Table C2, or a pharmaceutically acceptable salt thereof.
Embodiment 149. A pharmaceutical composition comprising a compound of any one of Embodiments 1-148, or a pharmaceutically acceptable salt thereof, and a pharmaceutically acceptable excipient.
Embodiment 150. A BCL-X_{L} protein non-covalently bound with a compound of any one of Embodiments 1-148, or a pharmaceutically acceptable salt thereof.
Embodiment 151. A ternary complex comprising a BCL-X_{L} protein, a compound of any one of Embodiments 1-148, or a pharmaceutically acceptable salt thereof, and a CRBN protein.
Embodiment 152. A method for treating a cancer in a subject in need thereof, the method comprising administering to the subject a therapeutically effective amount of a compound of any one of Embodiments 1-148, or a pharmaceutically acceptable salt thereof, or a pharmaceutical composition according to Embodiment 149.
Embodiment 153. The method of Embodiment 152, further comprising administering an additional therapy or therapeutic agent.
Embodiment 154. The method of Embodiment 153, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody, an ERK inhibitor, a FGFR1 inhibitor, a FGFR2 inhibitor, a FGFR3 inhibitor, a FGFR4 inhibitor, a HER2 inhibitor, a JAK2 inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, L-asparaginase, chemotherapy, radiotherapy, or a combination thereof.
Embodiment 155. The method of any one of Embodiments 152-154, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.
Embodiment 156. The method of Embodiment 155, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.
Embodiment 157. The method of Embodiment 155, wherein the cancer is lung cancer.
Embodiment 158. The method of Embodiment 157, wherein the lung cancer is small cell lung cancer (SCLC).
Embodiment 159. The method of Embodiment 158, comprising administering an additional therapy or therapeutic agent.
Embodiment 160. The method of Embodiment 159, wherein the additional therapy or therapeutic agent is a BCL-2 inhibitor.
Embodiment 161. The method of Embodiment 160, wherein the BCL-2 inhibitor is selected from the group consisting oflisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), ZN-d5, or a combination thereof.
Embodiment 162. The method of Embodiment 161, wherein the BCL-2 inhibitor is venetoclax.
Embodiment 163. A method for inducing degradation of a BCL-X_{L} protein in a mammalian cell, the method comprising contacting the mammalian cell with an effective amount of a compound of any one of Embodiments 1-148, or a pharmaceutically acceptable salt thereof.
Embodiment 164. The method of Embodiment 163, wherein the contacting occurs in vivo.
Embodiment 165. The method of Embodiment 163, wherein the contacting occurs in vitro.
Embodiment 166. The method of any one of Embodiments 163-165, wherein the mammalian cell is a mammalian cancer cell.

1.-32. (canceled)

33. A compound of Formula (I-i3): 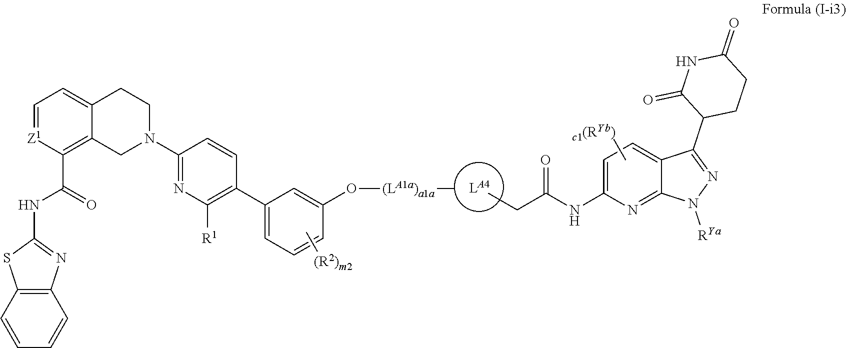
or a pharmaceutically acceptable salt thereof, wherein:
Z¹ is selected from the group consisting of: N and CH;
R¹ is —C(O)OH;
R² is CH₃ or CF₃;
m2 is 0 or 1;
a1a is an integer from 0 to 4;
each L^{A1a} is —CH₂;
L^{A4} is 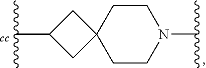
wherein cc represents point of attachment to -(L^{A1a})ₐ₁ₐ-;
c1 is 0, 1, or 2;
each R^{Yb} is independently selected from the group consisting of: —F, —OMe, —CH₃, —CHF₂, —CH₂F, and —CF₃; and
R^{Ya} is methyl.

34. The compound of claim 33, wherein a1a is 0 or 1.

35. The compound of claim 33, wherein m2 is 1; and R² is —CF₃.

36. The compound of claim 33, wherein m2 is 1; and R² is —CH₃.

37. The compound of claim 33, wherein the 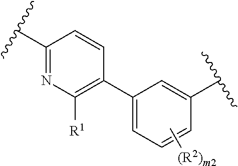
moiety is

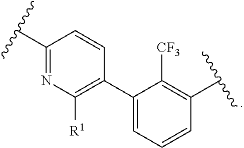

38. The compound of claim 33, wherein the 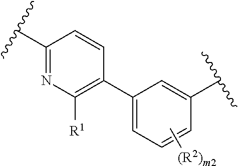
moiety is

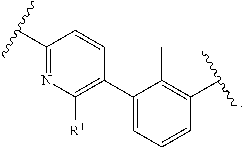

39. The compound of claim 33, wherein c1 is 0.

40. The compound of claim 33, wherein Z¹ is CH.

41. The compound of claim 33, wherein Z¹ is N.

42. The compound of claim 33, wherein the compound is: 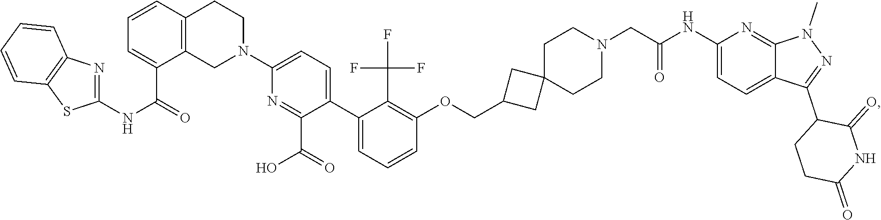 [SMILES: Cn1nc(C2CCC(=O)NC2=O)c2ccc(NC(=O)CN3CCC4(CC3)CC(COc3cccc(-c5ccc(N6CCc7cccc(C(=O)Nc8nc9ccccc9s8)c7C6)nc5C(=O)O)c3C(F)(F)F)C4)nc21]
or a pharmaceutically acceptable salt thereof.

43. The compound of claim 33, wherein the compound is: 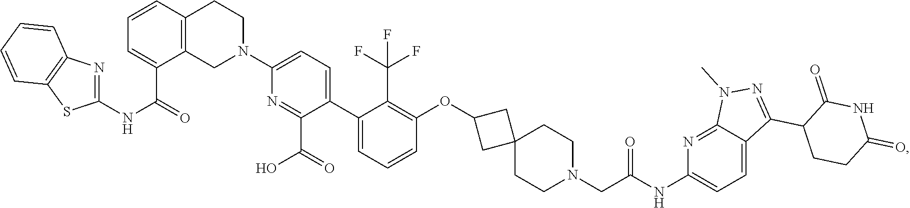 [SMILES: Cn1nc(C2CCC(=O)NC2=O)c2ccc(NC(=O)CN3CCC4(CC3)CC(Oc3cccc(-c5ccc(N6CCc7cccc(C(=O)Nc8nc9ccccc9s8)c7C6)nc5C(=O)O)c3C(F)(F)F)C4)nc21]
or a pharmaceutically acceptable salt thereof.

44. The compound of claim 33, wherein the compound is: 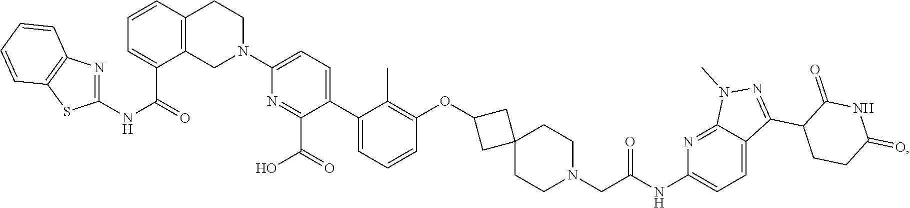 [SMILES: Cc1c(OC2CC3(CCN(CC(=O)Nc4ccc5c(C6CCC(=O)NC6=O)nn(C)c5n4)CC3)C2)cccc1-c1ccc(N2CCc3cccc(C(=O)Nc4nc5ccccc5s4)c3C2)nc1C(=O)O]
or a pharmaceutically acceptable salt thereof.

45. The compound of claim 33, wherein the compound is: 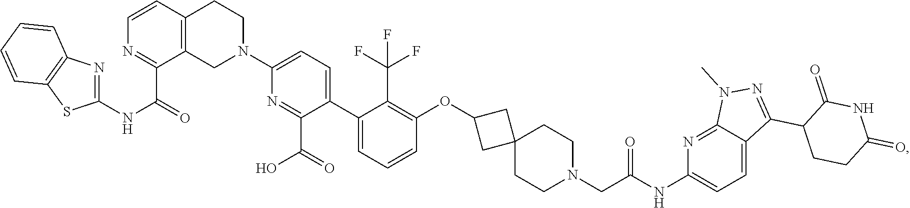 [SMILES: Cn1nc(C2CCC(=O)NC2=O)c2ccc(NC(=O)CN3CCC4(CC3)CC(Oc3cccc(-c5ccc(N6CCc7ccnc(C(=O)Nc8nc9ccccc9s8)c7C6)nc5C(=O)O)c3C(F)(F)F)C4)nc21]
or a pharmaceutically acceptable salt thereof.

46. The compound of claim 33, wherein the compound is: 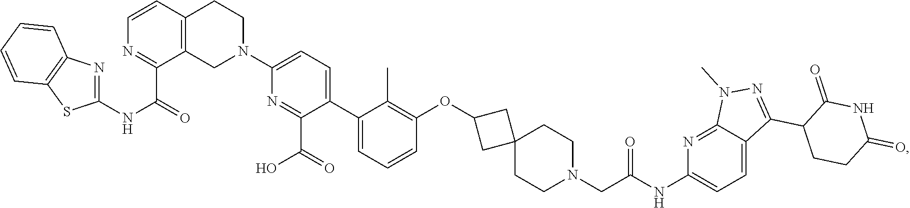 [SMILES: Cc1c(OC2CC3(CCN(CC(=O)Nc4ccc5c(C6CCC(=O)NC6=O)nn(C)c5n4)CC3)C2)cccc1-c1ccc(N2CCc3ccnc(C(=O)Nc4nc5ccccc5s4)c3C2)nc1C(=O)O]
or a pharmaceutically acceptable salt thereof.

47. A pharmaceutical composition comprising a compound of claim 33, or a pharmaceutically acceptable salt thereof, and a pharmaceutically acceptable excipient.

48. A method for treating a cancer in a subject in need thereof, the method comprising administering to the subject a therapeutically effective amount of a compound of claim 33, or a pharmaceutically acceptable salt thereof.

49. The method of claim 48, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.
